(19) United States
(12) Patent Application Publication
(10) Pub. No.: US 2024/0279215 A1
(43) Pub. Date: Aug. 22, 2024

(54) NAMPT MODULATORS

(71) Applicant: Cytokinetics, Incorporated, South San Francisco, CA (US)

(72) Inventors: Pu-Ping LU, Foster City, CA (US); Antonio ROMERO, San Mateo, CA (US); Wenyue WANG, Sunnyvale, CA (US); Alfredo GARCIA, San Francisco, CA (US); Minxing SHEN, San Bruno, CA (US); Christopher EVANS, Pacifica, CA (US); Scott E. COLLIBEE, San Carlos, CA (US); Todd TOCHIMOTO, Foster City, CA (US)

(21) Appl. No.: 18/393,327

(22) Filed: Dec. 21, 2023

Related U.S. Application Data
(60) Provisional application No. 63/434,849, filed on Dec. 22, 2022.

(51) Int. Cl. C07D 413/14; A61K 31/421; A61K 31/439; A61K 31/4439; A61K 31/444; A61K 31/454; C07D 213/56; C07D 265/32; C07D 295/03; C07D 401/14; C07D 413/12

(52) U.S. Cl. CPC C07D 413/14; A61K 31/421; A61K 31/439; A61K 31/4439; A61K 31/444; A61K 31/454; C07D 213/56; C07D 265/32; C07D 295/03; C07D 401/14; C07D 413/12

(57) ABSTRACT

Provided are compounds of Formula (I):

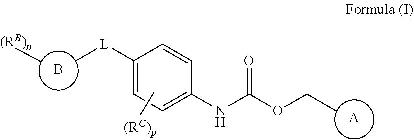

or a pharmaceutically acceptable salt thereof, wherein Ring A, Ring B, L, R^{B}, R^{C}, n, and p are as defined herein.

Also provided are pharmaceutically acceptable compositions comprising a compound of Formula (I), or a pharmaceutically acceptable salt thereof.

Also provided are methods of using a compound of Formula (I), or a pharmaceutically acceptable salt thereof.

### CROSS-REFERENCE TO RELATED APPLICATIONS

This application claims priority to and benefit of U.S. Provisional Patent Application No. 63/434,849 filed Dec. 22, 2022, the disclosure of which is hereby incorporated herein by reference in its entirety.

### FIELD

Provided herein are alkyl carbamate compounds, pharmaceutical compositions comprising such compounds, and methods of treating various diseases and conditions mediated by nicotinamide phosphoribosyltransferase (NAMPT) with such compounds.

### BACKGROUND

The present disclosure relates to the use of modulators of nicotinamide phosphoribosyltransferase (NAMPT) and derivatives thereof, as well as enhancers or inducers of NAMPT expression, NAMPT activity or NAMPT-mediated signaling for preventing or treating a variety of pathological conditions.

Nicotinamide adenine dinucleotide (NAD+) is an essential coenzyme (enzyme cofactor) involved in fundamental biological processes of both catabolic and anabolic metabolism. As a coenzyme, NAD is associated with many oxidative enzymes (typically dehydrogenases) involved in energy metabolism, serving as a universal electron carrier. NAD exists in cells in the oxidized state (NAD+ and NADP+), and the reduced state (NADH and NADPH), acting as a chemical means to capture and transfer free energy from oxidative processes in catabolism, or to provide small packets of energy to build macromolecules in anabolism. NADH produced from the oxidation of carbohydrates, lipids, and amino acids provides reducing equivalents to the electron transport chain of mitochondria, ultimately driving the synthesis of ATP in oxidative phosphorylation.

More than 200 enzymes use either NAD+ or NADP+ as a coenzyme, and the enzymatic functions are not limited to energy metabolism. It is now appreciated that NAD+ plays a role in regulating diverse functions, including mitochondrial function, respiratory capacity, and biogenesis, mitochondrial-nuclear signaling. Further, it controls cell signaling, gene expression, DNA repair, hematopoiesis, immune function, the unfolded protein response, and autophagy. Furthermore, NAD is anti-inflammatory and is the precursor for NADPH, which is the primary source of reducing power for combating oxidative stress. A large body of literature indicates that boosting NAD levels is an effective strategy to either prevent or ameliorate a wide variety of disease states (Strømland et al., Biochem Soc Trans. 2019, 47(1):119-130; Ralto et al., Nat Rev Nephrol. 2019; Fang et al., Trends Mol Med. 2017, 23(10):899-916; Yoshino et al., Cell Metab. 2011, 14(4):528-36; Yang and Sauve, Biochim Biophys Acta. 2016, 1864:1787-1800; Verdin, Science. 2015, 350(6265):1208-13).

Levels of NAD+ and NADP+-associated enzymes play important roles in normal physiology and are altered under various disease and stress conditions including aging. Cellular NAD+ levels decrease during aging, metabolic disease, inflammatory diseases, during ischemia/reperfusion injury, and in other conditions in humans (Massudi et al., PLoS ONE. 2012, 7(7): e42357) and animals (Yang et al., Cell. 2007, 130(6):1095-107; Braidy et al. PLoS One. 2011, 26; 6(4):e19194; Peek et al. Science. 2013, 342(6158):1243417; Ghosh et al., J Neurosci. 2012, 32(17):5821-32), suggesting that modulation of cellular NAD+ level affects the speed and severity of the decline and deterioration of bodily functions. Therefore, an increase in cellular NAD+ concentration could be beneficial in the context of aging and age-related diseases.

The cellular NAD+ pool is controlled by a balance between the activity of NAD+-synthesizing and consuming enzymes. In mammals, NAD+ is synthesized from a variety of dietary sources, including one or more of its major precursors that include: tryptophan (Trp), nicotinic acid (NA), nicotinamide riboside (NR), nicotinamide mononucleotide (NMN), and nicotinamide (NAM). Based upon the bioavailability of its precursors, there are three pathways for the synthesis of NAD+ in cells: (i) from Trp by the de novo biosynthesis pathway or kynurenine pathway (ii) from NA in the Preiss-Handler pathway and (iii) from NAM, NR, and NMN in the salvage pathway (Verdin et al., Science. 2015, 350(6265):1208-13). Of these, the predominant NAD+ biosynthetic pathway involves the step of synthesis of nicotinamide mononucleotide (NMN) using nicotinamide and 5′-phosphoribosyl-pyrophosphate by the rate-limiting enzyme nicotinamide phosphoribosyl-transferase (NAMPT) that is critical to determination of longevity and responses to a variety of stresses (Fulco et al, Dev Cell. 2008, 14(5):661-73; Imai, Curr Pharm Des. 2009, 15(1):20-8; Revollo et al., J Biol Chem. 2004, 279(49):50754-63; Revollo et al., Cell Metab. 2007, November; 6(5):363-75; van der Veer et al., J Biol Chem. 2007, 282(15):10841-5; Yang et al., Cell. 2007, 130(6):1095-107). Thus, increasing the rate of NAMPT catalysis by a small molecule activator would be an effective strategy to boost NAD levels and thereby address a broad spectrum of disease states. These include cardiac diseases, chemotherapy induced tissue damage, renal diseases, metabolic diseases, muscular diseases, neurological diseases and injuries, diseases caused by impaired stem cell function, DNA damage and primary mitochondrial disorders, and ocular diseases. NAMPT activators capable of CNS penetration also have the ability to boost NAD levels in the brain which is advantageous for treating CNS related diseases.

### SUMMARY

In one aspect, provided herein is a compound of Formula (I):

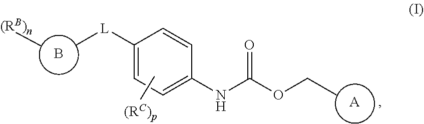

or a pharmaceutically acceptable salt thereof, wherein:
Ring A is: i) 5- to 6-membered heteroaryl optionally substituted with 1 to 4 R^{A}, or

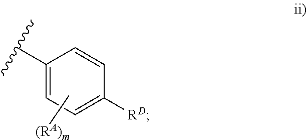

each R^{A} is independently selected from the group consisting of:
halogen;
cyano;
C₁-C₆ alkyl optionally substituted with 1 to 3 independently selected halogens or —OH; —O(C₁-C₆ alkyl) optionally substituted with 1 to 3 independently selected halogens; and —C(O)NR^{A1}R^{A2}, wherein R^{A1} and R^{A2} are each independently hydrogen or C₁-C₆ alkyl;
R^{D} is selected from the group consisting of:
halogen;
cyano;
C₁-C₆ alkyl optionally substituted with 1 to 3 independently selected halogens or —OH; —O(C₁-C₆ alkyl) optionally substituted with 1 to 3 independently selected halogens; and —C(O)NR^{A1}R^{A2}, wherein R^{A1} and R^{A2} are each independently hydrogen or C₁-C₆ alkyl;
L is selected from the group consisting of a bond, C₁-C₆ alkylene,
#—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$,
#—C(O)—N(R^{L})—(C₁-C₆ alkylene)-$, #—(C₁-C₆ alkylene)-C(O)—N(R^{L})—$,
#—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$, #—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl;
Ring B is 4- to 10-membered heterocycloalkyl, 3- to 8-membered cycloalkyl, 5- to 6-membered heteroaryl, or phenyl;
each R^{B} is independently selected from the group consisting of:
halogen;
—OH;
oxo;
cyano;
O(C₁-C₆ alkyl) optionally substituted with phenyl or 1 to 3 independently selected halogens;
—C(O)(C₁-C₆ alkyl);
—C(O)O(C₁-C₆ alkyl);
phenyl;
5- to 6-membered heteroaryl;
4- to 8-membered heterocycloalkyl;
3- to 8-membered cycloalkyl;
—C(O)(3- to 8-membered cycloalkyl) optionally substituted with 1 to 3 independently selected halogens;
—C(O)(4- to 8-membered heterocycloalkyl) optionally substituted with 1 to 3 independently selected halogens;
—S(O)₂(C₁-C₆ alkyl);
—S(O)₂(3- to 8-membered cycloalkyl);
—S(O)₂(4- to 8-membered heterocycloalkyl);
—C(O)NR^{B1}R^{B2};
—S(O)₂NR^{B1}R^{B2};
—NR^{C1}S(O)₂NR^{B1}R^{B2};
—(C═N—R^{C1})—NR^{B1}R^{B2};
—NR^{C1}—(C═N—R^{C1})—NR^{B1}R^{B2};
—NR^{C1}—(C═N—CN)—NR^{B1}R^{B2}; and
C₁-C₆ alkyl optionally substituted with 1 to 5 substituents independently selected from the group consisting of halogen, —OH, 4- to 8-membered heterocycloalkyl, and —O(C₁-C₆ alkyl);
wherein R^{C1}, R^{B1} and R^{B2} are each independently hydrogen or C₁-C₆ alkyl;
m is 0, 1, 2, 3, or 4;
n is 0, 1, 2, 3, 4, or 5;
R^{C} is halogen, cyano, C₁-C₆ alkyl, OH, —O(C₁-C₆ alkyl), or 3- to 8-membered cycloalkyl; and
p is 0, 1, 2, 3, or 4;
wherein:
a) when L is a bond, ring B is selected from the group consisting of

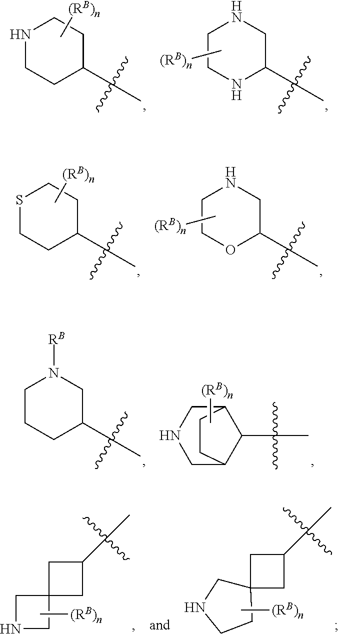

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

In one aspect, provided herein is a compound of Formula (I):

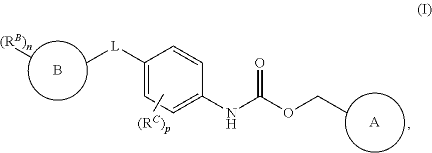

or a pharmaceutically acceptable salt thereof, wherein:
Ring A is: i) 5- to 6-membered heteroaryl optionally substituted with 1 to 4 R^{A}, or

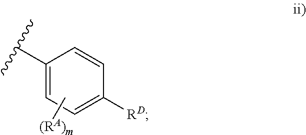

each R^{A} is independently selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl; R^{D} is selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
L is selected from the group consisting of a bond, C₁-C₆ alkylene,
#—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$,
#—C(O)—N(R^{L})—(C₁-C₆ alkylene)-$, #—(C₁-C₆ alkylene)-C(O)—N(R^{L})—$,
#—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$, #—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl;
Ring B is 4- to 10-membered heterocycloalkyl, 3- to 8-membered cycloalkyl, 5- to 6-membered heteroaryl, or phenyl;
each R^{B} is independently selected from the group consisting of:
halogen;
—OH;
oxo;
cyano;
—O(C₁-C₆ alkyl) optionally substituted with phenyl or 1 to 3 independently selected halogens;
—C(O)(C₁-C₆ alkyl);
—C(O)O(C₁-C₆ alkyl);
phenyl;
5- to 6-membered heteroaryl;
4- to 8-membered heterocycloalkyl;
3- to 8-membered cycloalkyl;
—C(O)(3- to 8-membered cycloalkyl);
—C(O)(4-8-membered heterocycloalkyl);
—S(O)₂(C₁-C₆ alkyl);
—S(O)₂(3- to 8-membered cycloalkyl);
—C(O)NR^{B1}R^{B2};
—S(O)₂NR^{B1}R^{B2};
—NR^{C1}S(O)₂NR^{B1}R^{B2};
—(C═N—R^{C1})—NR^{B1}R^{B2};
—NR^{C1}—(C═N—R^{C1})—NR^{B1}R^{B2};
—NR^{C1}—(C═N—CN)—NR^{B1}R^{B2}; and
C₁-C₆ alkyl optionally substituted with 1 to 5 substituents independently selected from the group consisting of halogen, —OH, and —O(C₁-C₆ alkyl);
wherein R^{C1}, R^{B1} and R^{B2} are each independently hydrogen or C₁-C₆ alkyl;
m is 0, 1, 2, 3, or 4;
n is 0, 1, 2, 3, 4, or 5;
R^{C} is halogen, cyano, C₁-C₆ alkyl, OH, —O(C₁-C₆ alkyl), or 3- to 8-membered cycloalkyl; and
p is 0, 1, 2, 3, or 4;
wherein:
a) when L is a bond, ring B is selected from the group consisting of

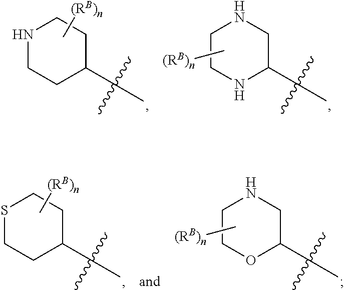

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

In another aspect, provided is a compound of Table 1, or a pharmaceutically acceptable salt thereof.

In a further aspect, provided herein are pharmaceutical compositions comprising at least one compound of Formula (I) or of Table 1, or a pharmaceutically acceptable salt of any of the foregoing, optionally further comprising a pharmaceutically acceptable excipient.

In another aspect, provided herein is a method of treating a disease or condition mediated by NAMPT activity in a subject in need thereof, comprising administering to the subject an effective amount of at least one compound of Formula (I), such as a compound of Table 1, or a pharmaceutically acceptable salt thereof, or a pharmaceutical composition comprising at least one compound of Formula (I). In some embodiments, the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder. In some embodiments, the disease or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

Additional embodiments, features, and advantages of the present disclosure will be apparent from the following detailed description and through practice of the present disclosure.

For the sake of brevity, the disclosures of publications cited in this specification, including patents, are herein incorporated by reference.

### DETAILED DESCRIPTION

### Definitions

As used in the present specification, the following words and phrases are generally intended to have the meanings as set forth below, except to the extent that the context in which they are used indicates otherwise.

Throughout this application, unless the context indicates otherwise, references to a compound of Formula (I) includes all subgroups of Formula (I) defined herein, including all substructures, subgenera, preferences, embodiments, examples and particular compounds defined and/or described herein. References to a compound of Formula (I) and subgroups thereof include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula (I) and subgroups thereof include polymorphs, solvates, co-crystals, isomers, tautomers and/or oxides thereof. In some embodiments, references to a compound of Formula (I) and subgroups thereof include polymorphs, solvates, and/or co-crystals thereof. In some embodiments, references to a compound of Formula (I) and subgroups thereof include isomers, tautomers and/or oxides thereof. In some embodiments, references to a compound of Formula (I) and subgroups thereof, include solvates thereof. Similarly, the term “salts” includes solvates of salts of compounds.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

As used herein, the term “alkylene” refers to divalent alkyl group as defined herein above having 1 to 20 carbon atoms. Unless otherwise provided, alkylene refers to moieties having 1 to 20 carbon atoms, 1 to 16 carbon atoms, 1 to 10 carbon atoms, 1 to 7 carbon atoms, or 1 to 4 carbon atoms. Alkylene groups include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, and n-decylene.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic), spiro, branched, and/or bridged. Examples of cycloalkyl groups include cyclopropyl, cyclobutyl, cyclopentyl, and cyclohexyl, as well as bridged, caged, and spirocyclic ring groups (e.g., norbornane, bicyclo[2.2.2]octane, spiro[3.3]heptane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group. Examples of polycyclic heteroaryl groups consisting of a heteroaryl ring fused to a non-aromatic ring are described below.

“Heterocycloalkyl” indicates a non-aromatic, fully saturated ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon. Heterocycloalkyl groups may be monocyclic or polycyclic (e.g., bicyclic, tricyclic), spiro, branched, and/or bridged. Examples of heterocycloalkyl groups include oxiranyl, aziridinyl, azetidinyl, pyrrolidinyl, imidazolidinyl, pyrazolidinyl, piperidinyl, piperazinyl, morpholinyl and thiomorpholinyl. Examples include thiomorpholine S-oxide and thiomorpholine S,S-dioxide. Examples of spirocyclic heterocycloalkyl groups include azaspiro[3.3]heptane, diazaspiro[3.3]heptane, diazaspiro[3.4]octane, and diazaspiro[3.5]nonane. In addition, one ring of a polycyclic heterocycloalkyl group may be aromatic (e.g., aryl or heteroaryl), provided the polycyclic heterocycloalkyl group is bound to the parent structure via a non-aromatic carbon or nitrogen atom. For example, a 1,2,3,4-tetrahydroquinolin-1-yl group (wherein the moiety is bound to the parent structure via a non-aromatic nitrogen atom) is considered a heterocycloalkyl group, while 1,2,3,4-tetrahydroquinolin-8-yl group (wherein the moiety is bound to the parent structure via an aromatic carbon atom) is not considered a heterocycloalkyl group. Examples of polycyclic heterocycloalkyl groups consisting of a heterocycloalkyl group fused to an aromatic ring are described below.

“Heterocycloalkenyl” indicates a non-aromatic ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon, and at least one double bond derived by the removal of one molecule of hydrogen from adjacent carbon atoms, adjacent nitrogen atoms, or adjacent carbon and nitrogen atoms of the corresponding heterocycloalkyl. Heterocycloalkenyl groups may be monocyclic or polycyclic (e.g., bicyclic, tricyclic). Examples of heterocycloalkenyl groups include dihydrofuranyl (e.g., 2,3-dihydrofuranyl, 2,5-dihydrofuranyl), dihydrothiophenyl (e.g., 2,3-dihydrothiophenyl, 2,5-dihydrothiophenyl), dihydropyrrolyl (e.g., 2,3-dihydro-1H-pyrrolyl, 2,5-dihydro-1H-pyrrolyl), dihydroimidazolyl (e.g., 2,3-dihydro-1H-imidazolyl, 4,5-dihydro-1H-imidazolyl), pyranyl, dihydropyranyl (e.g., 3,4-dihydro-2H-pyranyl, 3,6-dihydro-2H-pyranyl), tetrahydropyridinyl (e.g., 1,2,3,4-tetrahydropyridinyl, 1,2,3,6-tetrahydropyridinyl) and dihydropyridine (e.g., 1,2-dihydropyridine, 1,4-dihydropyridine). In addition, one ring of a polycyclic heterocycloalkenyl group may be aromatic (e.g., aryl or heteroaryl), provided the polycyclic heterocycloalkenyl group is bound to the parent structure via a non-aromatic carbon or nitrogen atom. For example, a 1,2-dihydroquinolin-1-yl group (wherein the moiety is bound to the parent structure via a non-aromatic nitrogen atom) is considered a heterocycloalkenyl group, while 1,2-dihydroquinolin-8-yl group (wherein the moiety is bound to the parent structure via an aromatic carbon atom) is not considered a heterocycloalkenyl group. Examples of polycyclic heterocycloalkenyl groups consisting of a heterocycloalkenyl group fused to an aromatic ring are described below.

Examples of polycyclic rings consisting of an aromatic ring (e.g., aryl or heteroaryl) fused to a non-aromatic ring (e.g., cycloalkyl, cycloalkenyl, heterocycloalkyl, heterocycloalkenyl) include indenyl, 2,3-dihydro-1H-indenyl, 1,2,3,4-tetrahydronaphthalenyl, benzo[1,3]dioxolyl, tetrahydroquinolinyl, 2,3-dihydrobenzo[1,4]dioxinyl, indolinyl, isoindolinyl, 2,3-dihydro-1H-indazolyl, 2,3-dihydro-1H-benzo[d]imidazolyl, 2,3-dihydrobenzofuranyl, 1,3-dihydroisobenzofuranyl, 1,3-dihydrobenzo[c]isoxazolyl, 2,3-dihydrobenzo[d]isoxazolyl, 2,3-dihydrobenzo[d]oxazolyl, 2,3-dihydrobenzo[b]thiophenyl, 1,3-dihydrobenzo[c]thiophenyl, 1,3-dihydrobenzo[c]isothiazolyl, 2,3-dihydrobenzo[d]isothiazolyl, 2,3-dihydrobenzo[d]thiazolyl, 5,6-dihydro-4H-cyclopenta[d]thiazolyl, 4,5,6,7-tetrahydrobenzo[d]thiazolyl, 5,6-dihydro-4H-pyrrolo[3,4-d]thiazolyl, 4,5,6,7-tetrahydrothiazolo[5,4-c]pyridinyl, indolin-2-one, indolin-3-one, isoindolin-1-one, 1,2-dihydroindazol-3-one, 1H-benzo[d]imidazol-2(3H)-one, benzofuran-2(3H)-one, benzofuran-3(2H)-one, isobenzofuran-1(3H)-one, benzo[c]isoxazol-3(1H)-one, benzo[d]isoxazol-3(2H)-one, benzo[d]oxazol-2(3H)-one, benzo[b]thiophen-2(3H)-one, benzo[b]thiophen-3(2H)-one, benzo[c]thiophen-1(3H)-one, benzo[c]isothiazol-3(1H)-one, benzo[d]isothiazol-3(2H)-one, benzo[d]thiazol-2(3H)-one, 4,5-dihydropyrrolo[3,4-d]thiazol-6-one, 1,2-dihydropyrazolo[3,4-d]thiazol-3-one, quinolin-4(3H)-one, quinazolin-4(3H)-one, quinazoline-2,4(1H,3H)-dione, quinoxalin-2(1H)-one, quinoxaline-2,3(1H,4H)-dione, cinnolin-4(3H)-one, pyridin-2(1H)-one, pyrimidin-2(1H)-one, pyrimidin-4(3H)-one, pyridazin-3(2H)-one, 1H-pyrrolo[3,2-b]pyridin-2(3H)-one, 1H-pyrrolo[3,2-c]pyridin-2(3H)-one, 1H-pyrrolo[2,3-c]pyridin-2(3H)-one, 1H-pyrrolo[2,3-b]pyridin-2(3H)-one, 1,2-dihydropyrazolo[3,4-d]thiazol-3-one and 4,5-dihydropyrrolo[3,4-d]thiazol-6-one. As discussed herein, whether each ring is considered an aryl, heteroaryl, cycloalkyl, cycloalkenyl, heterocycloalkyl or heterocycloalkenyl group is determined by the atom through which the moiety is bound to the parent structure.

“Halogen” or “halo” refers to fluorine, chlorine, bromine or iodine.

Unless otherwise indicated, compounds disclosed and/or described herein include all possible enantiomers, diastereomers, meso isomers and other stereoisomeric forms, including racemic mixtures, optically pure forms and intermediate mixtures thereof. Enantiomers, diastereomers, meso isomers and other stereoisomeric forms can be prepared using chiral synthons or chiral reagents, or resolved using conventional techniques. Unless specified otherwise, when the compounds disclosed and/or described herein contain olefinic double bonds or other centers of geometric asymmetry, it is intended that the compounds include both E and Z isomers. When the compounds described herein contain moieties capable of tautomerization, and unless specified otherwise, it is intended that the compounds include all possible tautomers.

“Protecting group” has the meaning conventionally associated with it in organic synthesis, i.e., a group that selectively blocks one or more reactive sites in a multifunctional compound such that a chemical reaction can be carried out selectively on another unprotected reactive site, and such that the group can readily be removed after the selective reaction is complete. A variety of protecting groups are disclosed, for example, in T.H. Greene and P. G. M. Wuts, Protective Groups in Organic Synthesis, Third Edition, John Wiley & Sons, New York (1999). For example, a “hydroxy protected form” contains at least one hydroxy group protected with a hydroxy protecting group. Likewise, amines and other reactive groups may similarly be protected.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66(1), 1-19). Those skilled in the art will recognize various synthetic methodologies that may be used to prepare pharmaceutically acceptable addition salts.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

The term “substituted” means that the specified group or moiety bears one or more substituents including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocycloalkyl, heterocycloalkenyl, aralkyl, aminosulfonyl, sulfonylamino, sulfonyl, oxo, carbonylalkylenealkoxy and the like. The term “unsubstituted” means that the specified group bears no substituents. Where the term “substituted” is used to describe a structural system, the substitution is meant to occur at any valency-allowed position on the system, including at valency-allowed positions that are heteroatomic. For example, an “optionally substituted piperazin-2-yl” group can be substituted at any valency-allowed position, including nitrogen.

When a group with variable substitution is depicted, such as

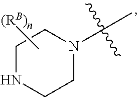

the indicated substituent can be at any valency-allowed position, including the —NH—, as in

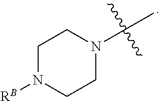

Furthermore, when a bicyclic group with variable substitution is depicted, the indicated substituent can be at any valency-allowed position at either ring or at both rings. For instance, the R^{B} substituent of

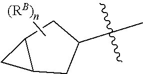

can be at either the cyclopentyl or the cyclopropyl component of the fused system, or it can be at both the cyclopentyl and the cyclopropyl components of the fused system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The compounds disclosed and/or described herein can be enriched isotopic forms, e.g., enriched in the content of ²H, ³H, ¹¹C, ¹³C and/or ¹⁴C. In one embodiment, the compound contains at least one deuterium atom. Such deuterated forms can be made, for example, by the procedure described in U.S. Pat. Nos. 5,846,514 and 6,334,997. Such deuterated compounds may improve the efficacy and increase the duration of action of compounds disclosed and/or described herein. Deuterium substituted compounds can be synthesized using various methods, such as those described in: Dean, D., Recent Advances in the Synthesis and Applications of Radiolabeled Compounds for Drug Discovery and Development, Curr. Pharm. Des., 2000; 6(10); Kabalka, G. et al., The Synthesis of Radiolabeled Compounds via Organometallic Intermediates, Tetrahedron, 1989, 45(21), 6601-21; and Evans, E., Synthesis of radiolabeled compounds, J. Radioanal. Chem., 1981, 64(1-2), 9-32.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal, bird, or fish. In some embodiments, the patient or subject is a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

As used herein, the term “therapeutic” refers to the ability to modulate nicotinamide phosphoribosyltransferase (NAMPT). As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the chemical entity with the target or due to the interaction of the chemical entity with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of nicotinamide phosphoribosyltransferase (NAMPT). The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat”, “treated”, “treating”) includes one or more of: slowing or arresting the development of clinical symptoms of a disease or disorder; and/or relieving a disease or disorder (i.e., causing relief from or regression of clinical symptoms). The term covers both complete and partial reduction or prevention of the condition or disorder, and complete or partial reduction of clinical symptoms of a disease or disorder. Thus, compounds described and/or disclosed herein may prevent an existing disease or disorder from worsening, assist in the management of the disease or disorder, or reduce or eliminate the disease or disorder.

### Compounds

Compounds and salts thereof (such as pharmaceutically acceptable salts) are detailed herein, including in the Brief Summary and in the appended claims. Also provided are the use of all of the compounds described herein, including any and all stereoisomers, including geometric isomers (cis/trans), E/Z isomers, enantiomers, diastereomers, and mixtures thereof in any ratio including racemic mixtures, salts and solvates of the compounds described herein, as well as methods of making such compounds. Any compound described herein may also be referred to as a drug.

In one aspect, provided herein is a compound of Formula (I):

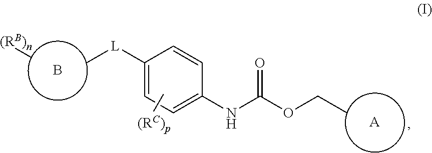

or a pharmaceutically acceptable salt thereof, wherein:
Ring A is: i) 5- to 6-membered heteroaryl optionally substituted with 1 to 4 R^{A}, or

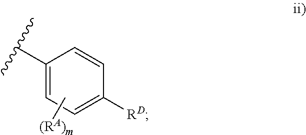

each R^{A} is independently selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
R^{D} is selected from the group consisting of:
halogen;
cyano; C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
L is selected from the group consisting of a bond, C₁-C₆ alkylene,
#—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$,
#—C(O)—N(R^{L})—(C₁-C₆ alkylene)-$, #—(C₁-C₆ alkylene)-C(O)—N(R^{L})—$,
#—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$, #—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl;
Ring B is 4- to 10-membered heterocycloalkyl, 3- to 8-membered cycloalkyl, 5- to 6-membered heteroaryl, or phenyl;
each R^{B} is independently selected from the group consisting of:
halogen;
—OH;
oxo;
cyano;
—O(C₁-C₆ alkyl) optionally substituted with phenyl or 1 to 3 independently selected halogens;
—C(O)(C₁-C₆ alkyl);
—C(O)O(C₁-C₆ alkyl);
phenyl;
5- to 6-membered heteroaryl;
4- to 8-membered heterocycloalkyl;
3- to 8-membered cycloalkyl;
—C(O)(3- to 8-membered cycloalkyl) optionally substituted with 1 to 3 independently selected halogens;
—C(O)(4- to 8-membered heterocycloalkyl) optionally substituted with 1 to 3 independently selected halogens;
—S(O)₂(C₁-C₆ alkyl);
—S(O)₂(3- to 8-membered cycloalkyl);
—S(O)₂(4- to 8-membered heterocycloalkyl);
—C(O)NR^{B1}R^{B2};
—S(O)₂NR^{B1}R^{B2};
—NR^{C1}S(O)₂NR^{B1}R^{B2};
—(C═N—R^{C1})—NR^{B1}R^{B2};
—NR^{C1}—(C═N—R^{C1})—NR^{B1}R^{B2};
—NR^{C1}—(C═N—CN)—NR^{B1}R^{B2}; and
C₁-C₆ alkyl optionally substituted with 1 to 5 substituents independently selected from the group consisting of halogen, —OH, 4- to 8-membered heterocycloalkyl, and —O(C₁-C₆ alkyl);
wherein R^{C1}, R^{B1} and R^{B2} are each independently hydrogen or C₁-C₆ alkyl;
m is 0, 1, 2, 3, or 4;
n is 0, 1, 2, 3, 4, or 5;
R^{C} is halogen, cyano, C₁-C₆ alkyl, OH, —O(C₁-C₆ alkyl), or 3- to 8-membered cycloalkyl; and
p is 0, 1, 2, 3, or 4;
wherein:
a) when L is a bond, ring B is selected from the group consisting of

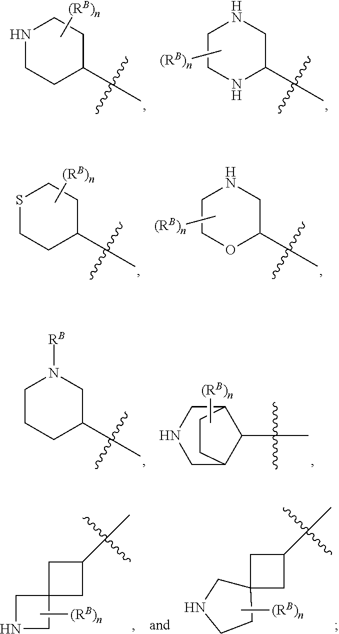

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

In one aspect, provided herein is a compound of Formula (I):

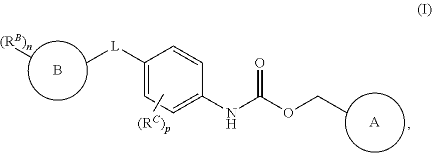

or a pharmaceutically acceptable salt thereof, wherein:
Ring A is: i) 5- to 6-membered heteroaryl optionally substituted with 1 to 4 R^{A}, or

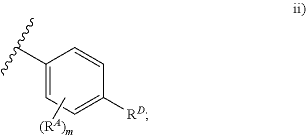

each R^{A} is independently selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
R^{D} is selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
L is selected from the group consisting of a bond, C₁-C₆ alkylene,
#—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$,
#—C(O)—N(R^{L})—(C₁-C₆ alkylene)-$, #—(C₁-C₆ alkylene)-C(O)—N(R^{L})—$,
#—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$, #—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl;
Ring B is 4- to 10-membered heterocycloalkyl, 3- to 8-membered cycloalkyl, 5- to 6-membered heteroaryl, or phenyl;
each R^{B} is independently selected from the group consisting of:
halogen;
—OH;
oxo;
cyano;
—O(C₁-C₆ alkyl) optionally substituted with phenyl or 1 to 3 independently selected halogens;
—C(O)(C₁-C₆ alkyl);
—C(O)O(C₁-C₆ alkyl);
phenyl;
5- to 6-membered heteroaryl;
4- to 8-membered heterocycloalkyl;
3- to 8-membered cycloalkyl;
—C(O)(3- to 8-membered cycloalkyl);
—C(O)(4-8-membered heterocycloalkyl);
—S(O)₂(C₁-C₆ alkyl);
—S(O)₂(3- to 8-membered cycloalkyl);
—C(O)NR^{B1}R^{B2};
—S(O)₂NR^{B1}R^{B2};
—NR^{C1}S(O)₂NR^{B1}R^{B2};
—(C═N—R^{C1})—NR^{B1}R^{B2};
—NR^{C1}—(C═N—R^{C1})—NR^{B1}R^{B2};
—NR^{C1}—(C═N—CN)—NR^{B1}R^{B2}; and
C₁-C₆ alkyl optionally substituted with 1 to 5 substituents independently selected from the group consisting of halogen, —OH, and —O(C₁-C₆ alkyl);
wherein R^{C1}, R^{B1} and R^{B2} are each independently hydrogen or C₁-C₆ alkyl;
m is 0, 1, 2, 3, or 4;
n is 0, 1, 2, 3, 4, or 5;
R^{C} is halogen, cyano, C₁-C₆ alkyl, OH, —O(C₁-C₆ alkyl), or 3- to 8-membered cycloalkyl; and
p is 0, 1, 2, 3, or 4;
wherein:
a) when L is a bond, ring B is selected from the group consisting of

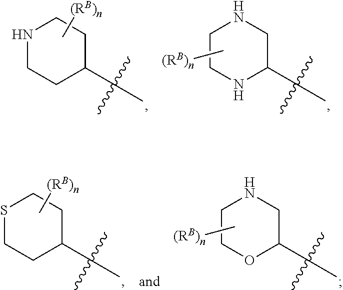

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

In some embodiments of a compound of Formula (I), Ring A is 5- to 6-membered heteroaryl optionally substituted with 1 to 4 R^{A}, or Ring A is

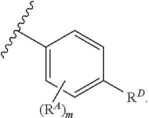

In some embodiments, Ring A is 5- to 6-membered heteroaryl optionally substituted with 1 to 4 R^{A}. In some embodiments, Ring A is

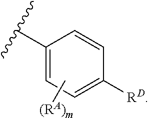

In some embodiments of a compound of Formula (I), Ring A is

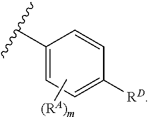

In some embodiments, R^{D} is halogen or —O(C₁-C₆ alkyl) optionally substituted with 1 to 3 independently selected halogens. In some embodiments, R^{D} is halogen. In some embodiments, R^{D} is halogen; cyano; C₁-C₆ alkyl optionally substituted with 1 to 3 independently selected halogens or —OH; —O(C₁-C₆ alkyl) optionally substituted with 1 to 3 independently selected halogens; or —C(O)NR^{A1}R^{A2}, wherein R^{A1} and R^{A2} are each independently hydrogen or C₁-C₆ alkyl. In some embodiments, R^{D} is halogen; cyano; C₁-C₃ alkyl optionally substituted with 1 to 3 independently selected halogens or —OH; —O(C₁-C₃ alkyl) optionally substituted with 1 to 3 independently selected halogens; or —C(O)NR^{A1}R^{A2}, wherein R^{A1} and R^{A2} are each independently hydrogen or C₁-C₃ alkyl. In some embodiments, R^{D} is chloro or fluoro; cyano; methyl optionally substituted with 1 to 3 independently selected chloro, fluoro, or —OH substituents; —OCH₃ optionally substituted with 1 to 3 independently selected chloro or fluoro substituents; or —C(O)NR^{A1}R^{A2}, wherein R^{A1} and R^{A2} are each independently hydrogen or methyl. In some embodiments, R^{D} is chloro or fluoro; cyano; —CH₃; methyl substituted with 1 to 3 independently selected fluoro or —OH substituents; —OCH₃ optionally substituted with 1 to 3 fluoro substituents; or —C(O)NH₂. In some embodiments, R^{D} is chloro or fluoro; cyano; —CH₃; —CH₂OH; —CHF₂; —OCH₃; —OCHF₂; or —C(O)NH₂. In some embodiments, R^{D} is chloro. In some embodiments, R^{D} is fluoro. In some embodiments, R^{D} is cyano. In some embodiments, R^{D} is —CH₂OH. In some embodiments, R^{D} is —CH₃. In some embodiments, R^{D} is —CH₂OH. In some embodiments, R^{D} is —CHF₂. In some embodiments, R^{D} is —OCH₃. In some embodiments, R^{D} is —OCHF₂. In some embodiments, R^{D} is —C(O)NH₂.

In some embodiments of a compound of Formula (I), Ring A is

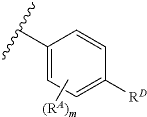

and R^{A} is halogen. In some embodiments, Ring A is

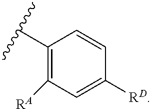

In some embodiments, Ring A is

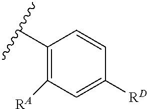

and R^{A} is halogen. In some embodiments, Ring A is

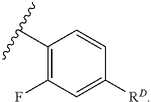

In some embodiments of a compound of Formula (I), Ring A, R^{A}, and R^{D} are taken together to form a moiety selected from the group consisting of:

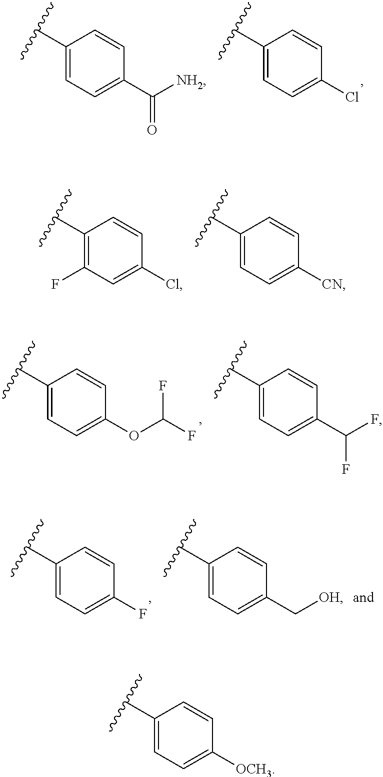

In some embodiments of a compound of Formula (I), Ring A, R^{A}, and R^{D} are taken together to form

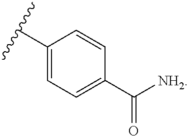 [SMILES: *c1ccc(C(N)=O)cc1]

In some embodiments of a compound of Formula (I), Ring A, R^{A}, and R^{D} are taken together to form

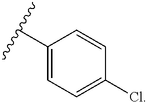 [SMILES: *c1ccc(Cl)cc1]

In some embodiments of a compound of Formula (I), Ring A, R^{A}, and R^{D} are taken together to form

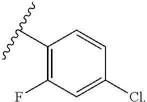 [SMILES: *c1ccc(Cl)cc1F]

In some embodiments of a compound of Formula (I), Ring A, R^{A}, and R^{D} are taken together to form

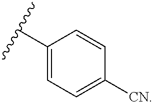 [SMILES: *c1ccc(C#N)cc1]

In some embodiments of a compound of Formula (I), Ring A, R^{A}, and R^{D} are taken together to form

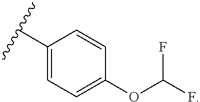 [SMILES: *c1ccc(OC(F)F)cc1]

In some embodiments of a compound of Formula (I), Ring A, R^{A}, and R^{D} are taken together to form

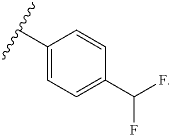 [SMILES: *c1ccc(C(F)F)cc1]

In some embodiments of a compound of Formula (I), Ring A, R^{A}, and R^{D} are taken together to form

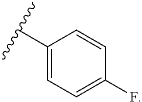 [SMILES: *c1ccc(F)cc1]

In some embodiments of a compound of Formula (I), Ring A, R^{A}, and R^{D} are taken together to form

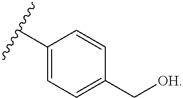 [SMILES: *c1ccc(CO)cc1]

In some embodiments of a compound of Formula (I), Ring A, R^{A} and R^{D} are taken together to form

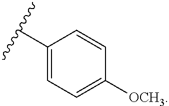 [SMILES: *c1ccc(OC)cc1]

In some embodiments of a compound of Formula (I), Ring A is 5- to 6-membered heteroaryl optionally substituted with 1 to 4 R^{A}.

In some embodiments of a compound of Formula (I), Ring A is 5-membered heteroaryl optionally substituted with 1 to 4 R^{A}. In some embodiments, Ring A is oxazolyl, pyrazolyl, thiazolyl, isothiazolyl, or isoxazolyl, each of which is optionally substituted with 1 to 3 R^{A}. In some embodiments, Ring A is oxazolyl optionally substituted with 1 to 3 R^{A}. In some embodiments, Ring A is oxazolyl optionally substituted with 1 to 2 R^{A}. In some embodiments, Ring A is pyrazolyl optionally substituted with 1 to 3 R^{A}. In some embodiments, Ring A is thiazolyl optionally substituted with 1 to 3 R^{A}. In some embodiments, Ring A is thiazolyl optionally substituted with 1 to 2 R^{A}. In some embodiments, Ring A is isothiazolyl optionally substituted with 1 to 3 R^{A}. In some embodiments, Ring A is isoxazolyl optionally substituted with 1 to 3 R^{A}. In some embodiments, Ring A is 1H-pyrazol-4-yl, isothiazol-4-yl, isothiazol-5-yl, isoxazol-4-yl, oxazol-5-yl, or thiazol-5-yl, each of which is optionally substituted with 1 to 3 R^{A}. In some embodiments, Ring A is 1H-pyrazol-4-yl optionally substituted with 1 to 3 R^{A}. In some embodiments, Ring A is isothiazol-4-yl optionally substituted with 1 to 3 R^{A}. In some embodiments, Ring A is isothiazol-5-yl optionally substituted with 1 to 3 R^{A}. In some embodiments, Ring A is isoxazol-4-yl optionally substituted with 1 to 3 R^{A}. In some embodiments, Ring A is oxazol-5-yl optionally substituted with 1 to 3 R^{A}. In some embodiments, Ring A is oxazol-5-yl optionally substituted with 1 to 2 R^{A}. In some embodiments, Ring A is thiazol-5-yl optionally substituted with 1 to 3 R^{A}. In some embodiments, Ring A is thiazol-5-yl optionally substituted with 1 to 2 R^{A}.

In some embodiments of a compound of Formula (I), Ring A is 5-membered heteroaryl optionally substituted with 1 to 4 R^{A}. In some embodiments, Ring A is oxazolyl, pyrazolyl, thiazolyl, isothiazolyl, or isoxazolyl, each of which is optionally substituted with 1 to 3 R^{A}. In some embodiments, Ring A is oxazolyl optionally substituted with 1 to 3 R^{A}. In some embodiments, Ring A is oxazolyl optionally substituted with 1 to 2 R^{A}. In some embodiments, Ring A is pyrazolyl optionally substituted with 1 to 3 R^{A}. In some embodiments, Ring A is thiazolyl optionally substituted with 1 to 3 R^{A}. In some embodiments, Ring A is isothiazolyl optionally substituted with 1 to 3 R^{A}. In some embodiments, Ring A is isoxazolyl optionally substituted with 1 to 3 R^{A}. In some embodiments, Ring A is 1H-pyrazol-4-yl, isothiazol-4-yl, isothiazol-5-yl, isoxazol-4-yl, oxazol-5-yl, or thiazol-5-yl, each of which is optionally substituted with 1 to 3 R^{A}. In some embodiments, Ring A is 1H-pyrazol-4-yl optionally substituted with 1 to 3 R^{A}. In some embodiments, Ring A is isothiazol-4-yl optionally substituted with 1 to 3 R^{A}. In some embodiments, Ring A is isothiazol-5-yl optionally substituted with 1 to 3 R^{A}. In some embodiments, Ring A is isoxazol-4-yl optionally substituted with 1 to 3 R^{A}. In some embodiments, Ring A is oxazol-5-yl optionally substituted with 1 to 3 R^{A}. In some embodiments, Ring A is oxazol-5-yl optionally substituted with 1 to 2 R^{A}. In some embodiments, Ring A is thiazol-5-yl optionally substituted with 1 to 3 R^{A}.

In some embodiments of a compound of Formula (I), Ring A is unsubstituted 5-membered heteroaryl. In some embodiments, Ring A is oxazolyl, pyrazolyl, thiazolyl, isothiazolyl, or isoxazolyl, each of which is unsubstituted.

In some embodiments of a compound of Formula (I), Ring A is 6-membered heteroaryl optionally substituted with 1 to 4 R^{A}. In some embodiments, Ring A is pyridinyl, pyridazinyl, or pyrimidinyl, each of which is optionally substituted with 1 to 4 R^{A}. In some embodiments, Ring A is pyridazinyl optionally substituted with 1 to 4 R^{A}. In some embodiments, Ring A is pyridinyl optionally substituted with 1 to 4 R^{A}. In some embodiments, Ring A is pyrimidinyl optionally substituted with 1 to 4 R^{A}. In some embodiments, Ring A is pyridazin-4-yl, pyridin-4-yl, or pyrimidin-4-yl, each of which is optionally substituted with 1 to 4 R^{A}. In some embodiments, Ring A is pyridazin-4-yl, optionally substituted with 1 to 4 R^{A}. In some embodiments, Ring A is pyridin-4-yl, optionally substituted with 1 to 4 R^{A}. In some embodiments, Ring A is pyrimidin-4-yl, optionally substituted with 1 to 4 R^{A}.

In some embodiments of a compound of Formula (I), R^{A} is halogen or C₁-C₆ alkyl optionally substituted with 1 to 3 independently selected halogens. In some embodiments, R^{A} is halogen or C₁-C₆ alkyl. In some embodiments, R^{A} is halogen. In some embodiments, R^{A} is halogen; cyano; C₁-C₆ alkyl optionally substituted with 1 to 3 independently selected halogens or —OH; —O(C₁-C₆ alkyl) optionally substituted with 1 to 3 independently selected halogens; or —C(O)NR^{A1}R^{A2}, wherein R^{A1} and R^{A2} are each independently hydrogen or C₁-C₆ alkyl. In some embodiments, R^{A} is halogen; cyano; C₁-C₃ alkyl optionally substituted with 1 to 3 independently selected halogens or —OH; —O(C₁-C₃ alkyl) optionally substituted with 1 to 3 independently selected halogens; or —C(O)NR^{A1}R^{A2}, wherein R^{A1} and R^{A2} are each independently hydrogen or C₁-C₃ alkyl. In some embodiments, R^{A} is chloro or fluoro; cyano; methyl optionally substituted with 1 to 3 independently selected chloro, fluoro, or —OH substituents; —OCH₃ optionally substituted with 1 to 3 independently selected chloro or fluoro substituents; or —C(O)NR^{A1}R^{A2} wherein R^{A1} and R^{A2} are each independently hydrogen or methyl. In some embodiments, R^{A} is chloro or fluoro; cyano; —CH₃; methyl substituted with 1 to 3 independently selected fluoro or —OH substituents; —OCH₃ optionally substituted with 1 to 3 fluoro substituents; or —C(O)NH₂. In some embodiments, R^{A} is chloro or fluoro; cyano; —CH₃; —CH₂OH; —CHF₂; —OCH₃; —OCHF₂; or —C(O)NH₂. In some embodiments, R^{A} is chloro. In some embodiments, R^{A} is fluoro. In some embodiments, R^{A} is cyano. In some embodiments, R^{A} is —CH₂OH. In some embodiments, R^{A} is —CH₃. In some embodiments, R^{A} is —CH₂OH. In some embodiments, R^{A} is —CHF₂. In some embodiments, R^{A} is —OCH₃. In some embodiments, R^{A} is —OCHF₂. In some embodiments, R^{A} is —C(O)NH₂.

In some embodiments, R^{A1} and R^{A1} are each independently hydrogen or C₁-C₆ alkyl. In some embodiments, R^{A1} and R^{A1} are each hydrogen. In some embodiments, R^{A1} and R^{A1} are each C₁-C₆ alkyl. In some embodiments, R^{A1} is hydrogen. In some embodiments, R^{A1} is C₁-C₆ alkyl. In some embodiments, R^{A1} is hydrogen. In some embodiments, R^{A1} is C₁-C₆ alkyl.

In some embodiments of a compound of Formula (I), Ring A and R^{A} are taken together to form a moiety selected from the group consisting of:

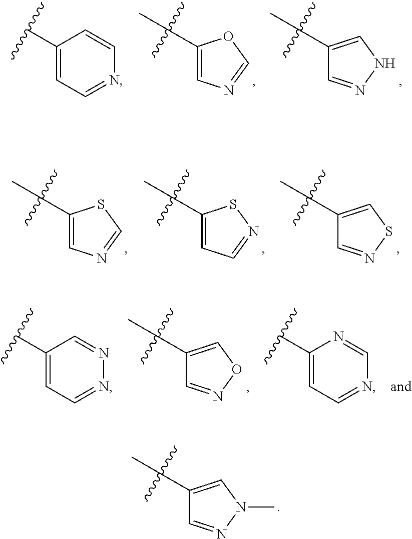

In some embodiments of a compound of Formula (I), Ring A and R^{A} are taken together to form

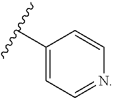 [SMILES: *c1ccncc1]

In some embodiments of a compound of Formula (I), Ring A and R^{A} are taken together to form

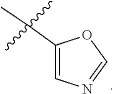 [SMILES: *c1cnco1]

In some embodiments of a compound of Formula (I), Ring A and R^{A} are taken together to form

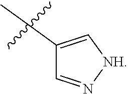 [SMILES: *c1cn[nH]c1]

In some embodiments of a compound of Formula (I), Ring A and R^{A} are taken together to form

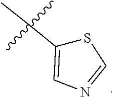 [SMILES: *c1cncs1]

In some embodiments of a compound of Formula (I), Ring A and R^{A} are taken together to form

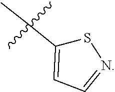 [SMILES: *c1ccns1]

In some embodiments of a compound of Formula (I), Ring A and R^{A} are taken together to form

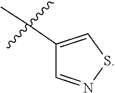 [SMILES: *c1cnsc1]

In some embodiments of a compound of Formula (I), Ring A and R^{A} are taken together to form

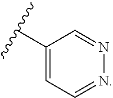 [SMILES: *c1ccnnc1]

In some embodiments of a compound of Formula (I), Ring A and R^{A} are taken together to form

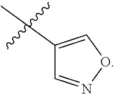 [SMILES: *c1cnoc1]

In some embodiments of a compound of Formula (I), Ring A and R^{A} are taken together to form

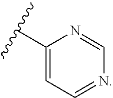 [SMILES: *c1ccncn1]

In some embodiments of a compound of Formula (I), Ring A and R^{A} are taken together to form

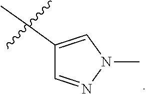 [SMILES: *c1cnn(C)c1]

In some embodiments of a compound of Formula (I), Ring B is 4- to 10-membered heterocycloalkyl, 3- to 8-membered cycloalkyl, 5- to 6-membered heteroaryl, or phenyl. In some embodiments, Ring B is 4- to 8-membered heterocycloalkyl, 3- to 8-membered cycloalkyl, or 5- to 6-membered heteroaryl. In some embodiments of a compound of Formula (I), Ring B is 4- to 8-membered heterocycloalkyl, 3- to 8-membered cycloalkyl, 5- to 6-membered heteroaryl, or phenyl. In some embodiments, Ring B is 4- to 8-membered heterocycloalkyl, 3- to 8-membered cycloalkyl, or 5- to 6-membered heteroaryl. In some embodiments, Ring B is 4- to 10-membered heterocycloalkyl. In some embodiments, Ring B is 4- to 8-membered heterocycloalkyl. In some embodiments, Ring B is 3- to 8-membered cycloalkyl. In some embodiments, Ring B is 5- to 6-membered heteroaryl. In some embodiments, Ring B is monocyclic. In some embodiments, ring B is polycyclic. In some embodiments, Ring B is spirocyclic. In some embodiments, Ring B is a bridged bicycle. In some embodiments, Ring B is a fused bicycle.

In some embodiments of a compound of Formula (I), Ring B is pyridinyl, piperazinyl, morpholinyl, piperidinyl, pyrrolidinyl, oxazolyl, or pyrazolyl. In some embodiments, Ring B is pyridinyl, piperazinyl, morpholinyl, piperidinyl, or pyrrolidinyl. In some embodiments, Ring B is

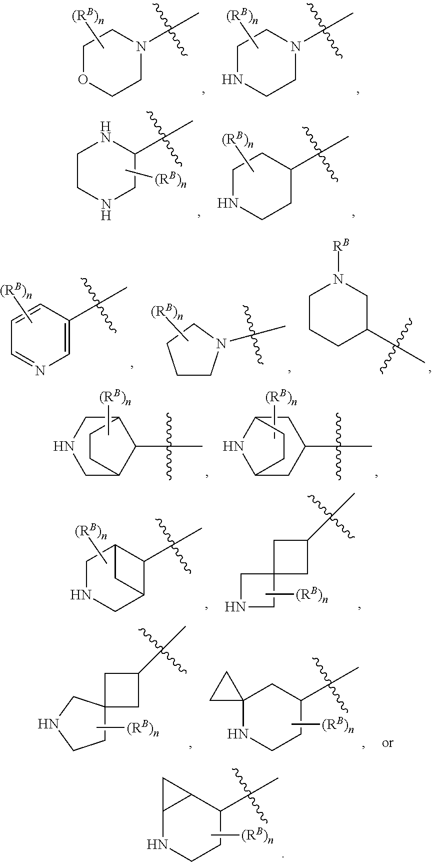

In some embodiments, Ring B is

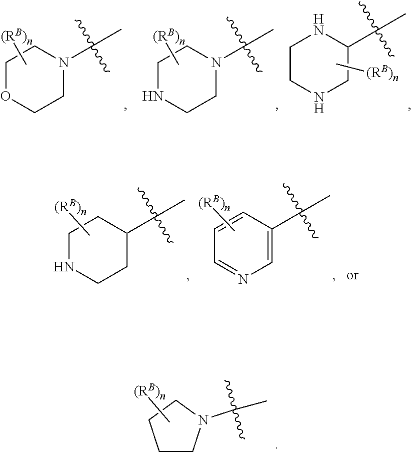

In some embodiments of a compound of Formula (I), Ring B is selected from the group consisting of: bicyclo[3.1.0]hexanyl; [3.3]heptanyl; 1,2,4-oxadiazolyl; 1,3,4-oxadiazolyl; pyrazolyl; pyrrolyl; 2,5-diazabicyclo[4.1.0]heptanyl; 2-azabicyclo[3.1.0]hexanyl; 3-azabicyclo[3.1.0]hexanyl; 5-azaspiro[2.4]heptanyl; 8-oxa-3-azabicyclo[3.2.1]octanyl; 2-azaspiro[3.3]heptanyl; 6-azaspiro[3.4]octanyl; 8-azabicyclo[3.2.1]octanyl; 4-azaspiro[2.5]octanyl; 3-azabicyclo[3.2.1]octanyl; 3-azabicyclo[3.1.1]heptanyl; 2-azabicyclo[4.1.0]heptanyl; azetidinyl; cyclobutyl; cyclohexyl; cyclopentyl; cyclopropyl; isothiazolyl; isoxazolyl; morpholinyl; oxazolyl; oxazolidinyl; oxetanyl; phenyl; piperazinyl; piperidinyl; pyrazinyl; pyridazinyl; pyridinyl; pyrimidinyl; pyrrolidinyl; tetrahydro-2H-pyranyl; tetrahydro-2H-thiopyranyl; tetrahydrofuranyl; and thiazolyl. In some embodiments, Ring B is bicyclo[3.1.0]hexanyl. In some embodiments, Ring B is [3.3]heptanyl. In some embodiments, Ring B is 2-azaspiro[3.3]heptanyl. In some embodiments, Ring B is 6-azaspiro[3.4]octanyl. In some embodiments, Ring B is 8-azabicyclo[3.2.1]octanyl. In some embodiments, Ring B is 4-azaspiro[2.5]octanyl. In some embodiments, Ring B is 3-azabicyclo[3.2.1]octanyl. In some embodiments, Ring B is 3-azabicyclo[3.1.1]heptanyl. In some embodiments, Ring B is 2-azabicyclo[4.1.0]heptanyl. In some embodiments, Ring B is 1,2,4-oxadiazolyl. In some embodiments, Ring B is 1,3,4-oxadiazolyl. In some embodiments, Ring B is pyrazolyl. In some embodiments, Ring B is pyrrolyl. In some embodiments, Ring B is 2,5-diazabicyclo[4.1.0]heptanyl. In some embodiments, Ring B is 2-azabicyclo[3.1.0]hexanyl. In some embodiments, Ring B is 3-azabicyclo[3.1.0]hexanyl. In some embodiments, Ring B is 5-azaspiro[2.4]heptanyl. In some embodiments, Ring B is 8-oxa-3-azabicyclo[3.2.1]octanyl. In some embodiments, Ring B is azetidinyl. In some embodiments, Ring B is cyclobutyl. In some embodiments, Ring B is cyclohexyl. In some embodiments, Ring B is cyclopentyl. In some embodiments, Ring B is cyclopropyl. In some embodiments, Ring B is isothiazolyl. In some embodiments, Ring B is isoxazolyl. In some embodiments, Ring B is morpholinyl. In some embodiments, Ring B is oxazolyl. In some embodiments, Ring B is oxazolidinyl. In some embodiments, Ring B is oxetanyl. In some embodiments, Ring B is phenyl. In some embodiments, Ring B is piperazinyl. In some embodiments, Ring B is piperidinyl. In some embodiments, Ring B is pyrazinyl. In some embodiments, Ring B is pyridazinyl. In some embodiments, Ring B is pyridinyl. In some embodiments, Ring B is pyrimidinyl. In some embodiments, Ring B is pyrrolidinyl. In some embodiments, Ring B is tetrahydro-2H-pyranyl. In some embodiments, Ring B is tetrahydro-2H-thiopyranyl. In some embodiments, Ring B is tetrahydrofuranyl. In some embodiments, Ring B is thiazolyl.

In some embodiments of a compound of Formula (I), Ring B is selected from the group consisting of: bicyclo[3.1.0]hexanyl; [3.3]heptanyl; 1,2,4-oxadiazolyl; 1,3,4-oxadiazolyl; pyrazolyl; pyrrolyl; 2,5-diazabicyclo[4.1.0]heptanyl; 2-azabicyclo[3.1.0]hexanyl; 3-azabicyclo[3.1.0]hexanyl; 5-azaspiro[2.4]heptanyl; 8-oxa-3-azabicyclo[3.2.1]octanyl; azetidinyl; cyclobutyl; cyclohexyl; cyclopentyl; cyclopropyl; isothiazolyl; isoxazolyl; morpholinyl; oxazolyl; oxazolidinyl; oxetanyl; phenyl; piperazinyl; piperidinyl; pyrazinyl; pyridazinyl; pyridinyl; pyrimidinyl; pyrrolidinyl; tetrahydro-2H-pyranyl; tetrahydro-2H-thiopyranyl; tetrahydrofuranyl; and thiazolyl. In some embodiments, Ring B is bicyclo[3.1.0]hexanyl. In some embodiments, Ring B is [3.3]heptanyl. In some embodiments, Ring B is 1,2,4-oxadiazolyl. In some embodiments, Ring B is 1,3,4-oxadiazolyl. In some embodiments, Ring B is pyrazolyl. In some embodiments, Ring B is pyrrolyl. In some embodiments, Ring B is 2,5-diazabicyclo[4.1.0]heptanyl. In some embodiments, Ring B is 2-azabicyclo[3.1.0]hexanyl. In some embodiments, Ring B is 3-azabicyclo[3.1.0]hexanyl. In some embodiments, Ring B is 5-azaspiro[2.4]heptanyl. In some embodiments, Ring B is 8-oxa-3-azabicyclo[3.2.1]octanyl. In some embodiments, Ring B is azetidinyl. In some embodiments, Ring B is cyclobutyl. In some embodiments, Ring B is cyclohexyl. In some embodiments, Ring B is cyclopentyl. In some embodiments, Ring B is cyclopropyl. In some embodiments, Ring B is isothiazolyl. In some embodiments, Ring B is isoxazolyl. In some embodiments, Ring B is morpholinyl. In some embodiments, Ring B is oxazolyl. In some embodiments, Ring B is oxazolidinyl. In some embodiments, Ring B is oxetanyl. In some embodiments, Ring B is phenyl. In some embodiments, Ring B is piperazinyl. In some embodiments, Ring B is piperidinyl. In some embodiments, Ring B is pyrazinyl. In some embodiments, Ring B is pyridazinyl. In some embodiments, Ring B is pyridinyl. In some embodiments, Ring B is pyrimidinyl. In some embodiments, Ring B is pyrrolidinyl. In some embodiments, Ring B is tetrahydro-2H-pyranyl. In some embodiments, Ring B is tetrahydro-2H-thiopyranyl. In some embodiments, Ring B is tetrahydrofuranyl. In some embodiments, Ring B is thiazolyl.

In some embodiments of a compound of Formula (I), Ring B is selected from the group consisting of:

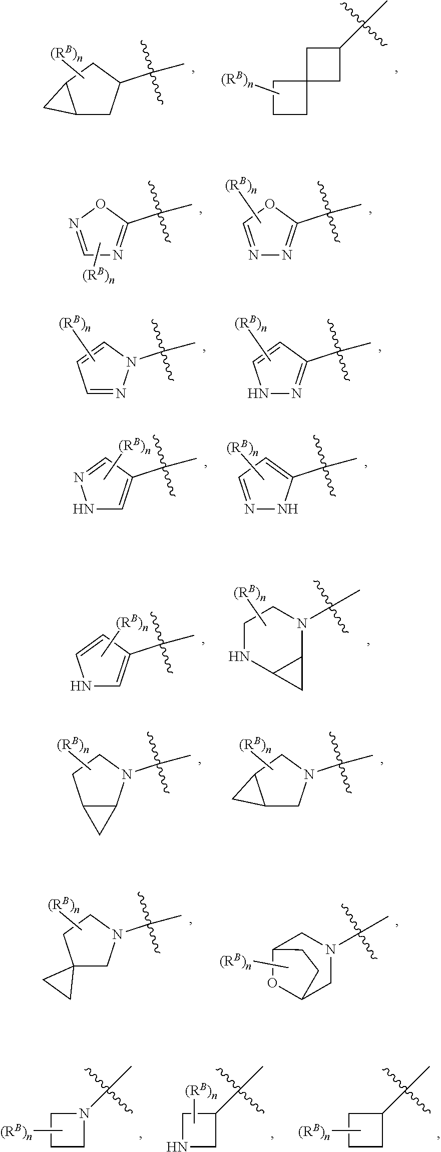

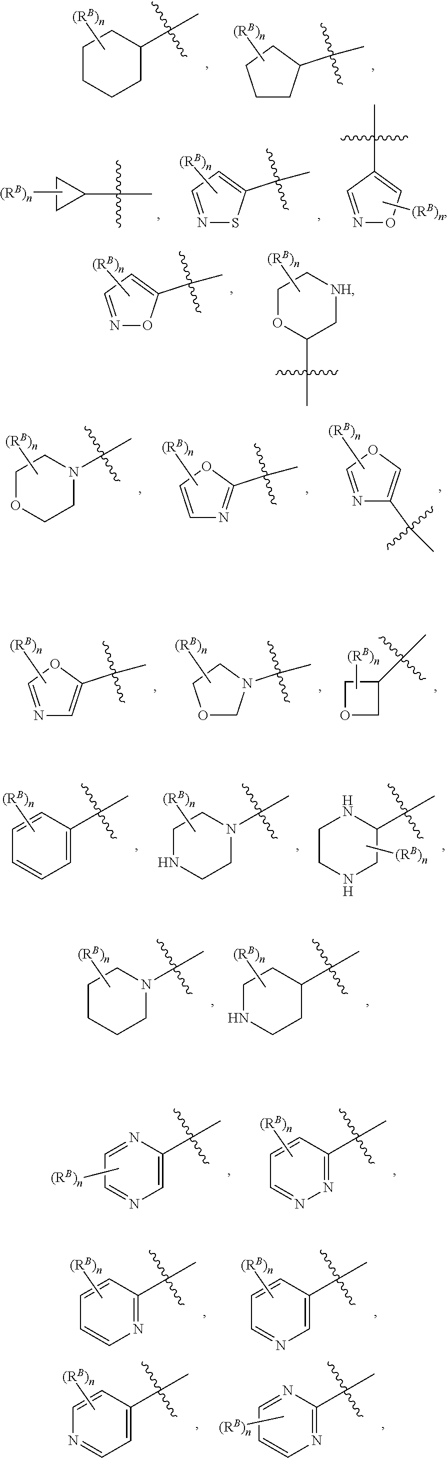

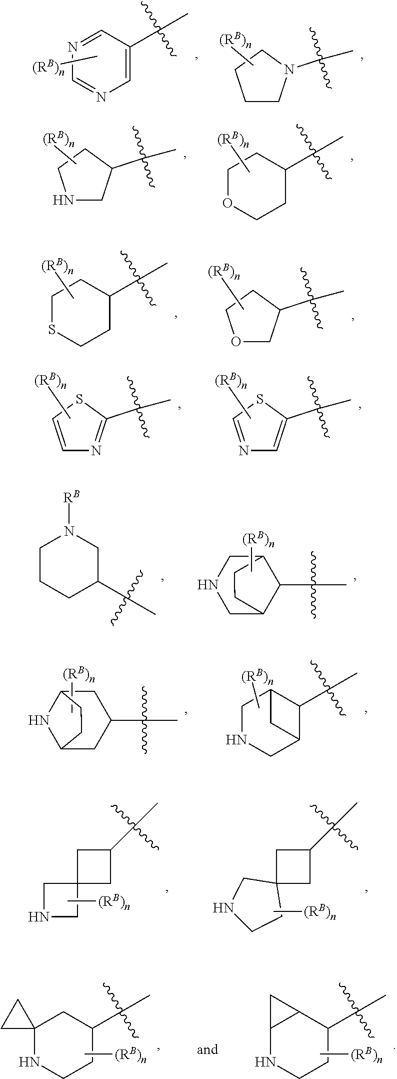

In some embodiments of a compound of Formula (I), Ring B is selected from the group consisting of:

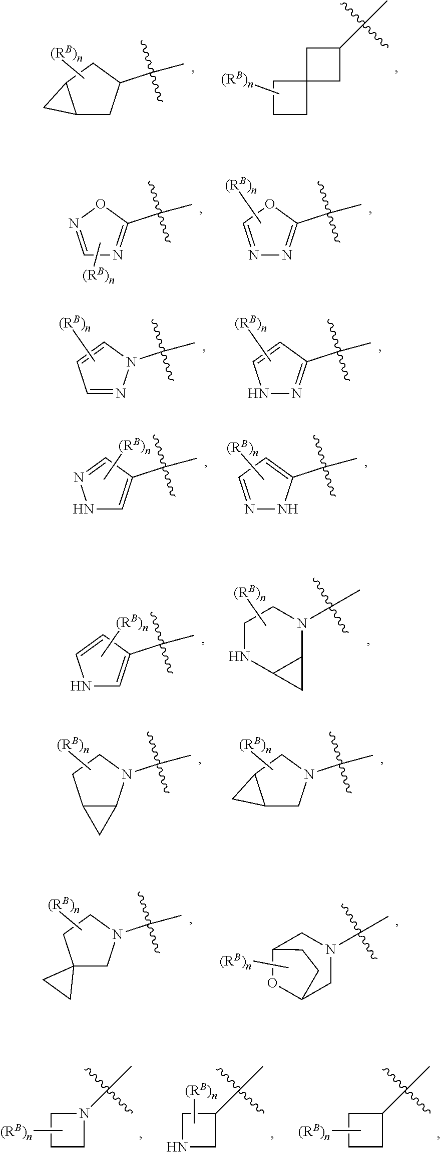

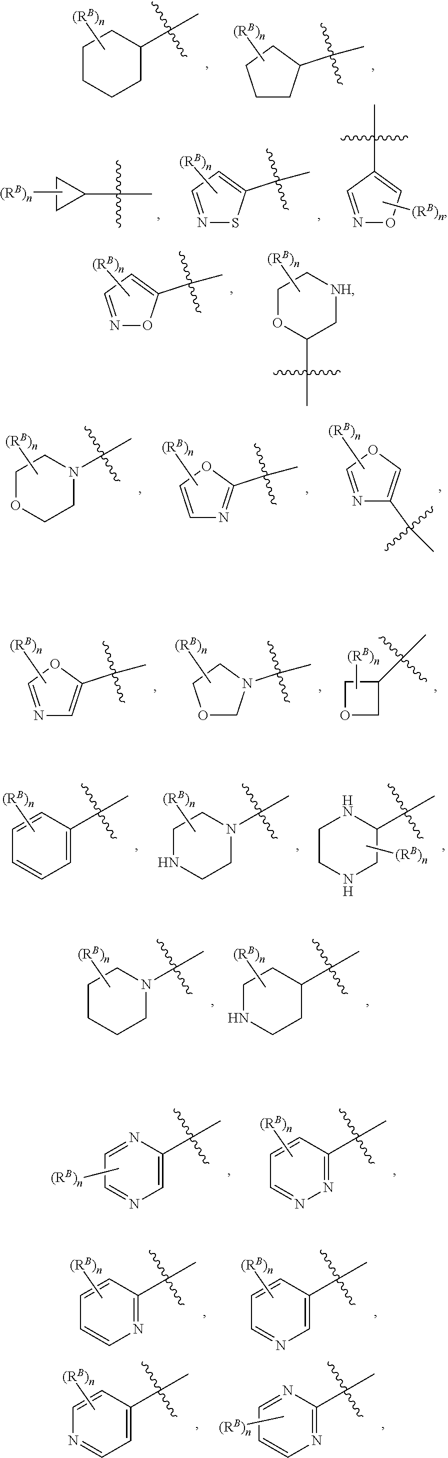

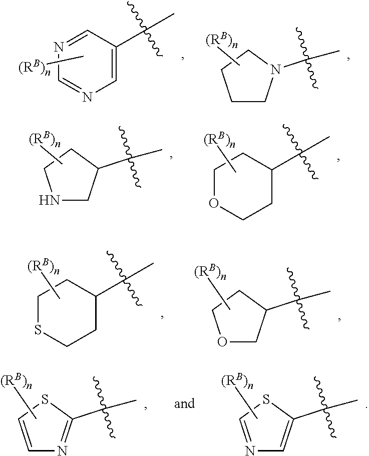

In some embodiments, Ring B is

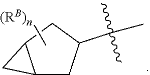

In some embodiments, Ring B is

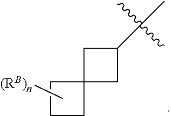

In some embodiments, Ring B is

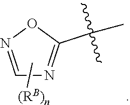

In some embodiments, Ring B is

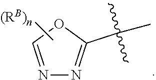

In some embodiments, Ring B is

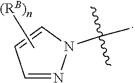

In some embodiments, Ring B is

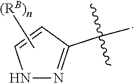

In some embodiments, Ring B is

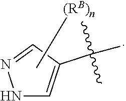

In some embodiments, Ring B is

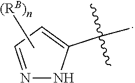

In some embodiments, Ring B is

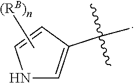

In some embodiments, Ring B is

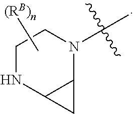

In some embodiments, Ring B is

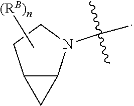

In some embodiments, Ring B is

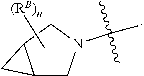

In some embodiments, Ring B is

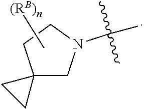

In some embodiments, Ring B is

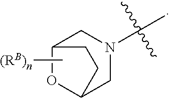

In some embodiments, Ring B is

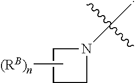

In some embodiments, Ring B is

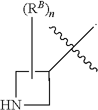

In some embodiments, Ring B is

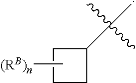

In some embodiments, Ring B is

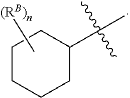

In some embodiments, Ring B is

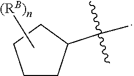

In some embodiments, Ring B is

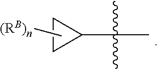

In some embodiments, Ring B is

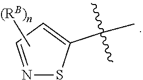

In some embodiments, Ring B is

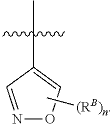

In some embodiments, Ring B is

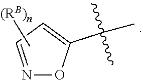

In some embodiments, Ring B is

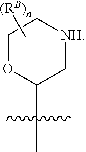

In some embodiments, Ring B is

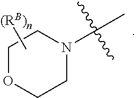

In some embodiments, Ring B is

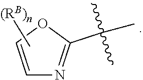

In some embodiments, Ring B is

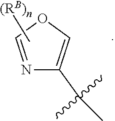

In some embodiments, Ring B is

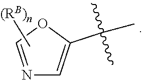

In some embodiments, Ring B is

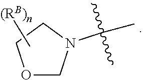

In some embodiments, Ring B is

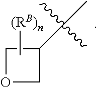

In some embodiments, Ring B is

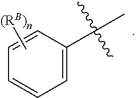

In some embodiments, Ring B is

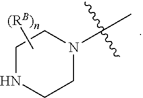

In some embodiments, Ring B is

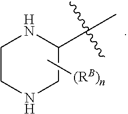

In some embodiments, Ring B is

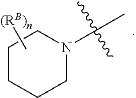

In some embodiments, Ring B is

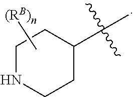

In some embodiments, Ring B is

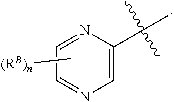

In some embodiments, Ring B is

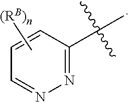

In some embodiments, Ring B is

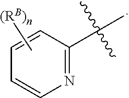

In some embodiments, Ring B is

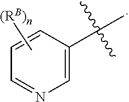

In some embodiments, Ring B is

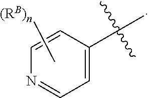

In some embodiments, Ring B is

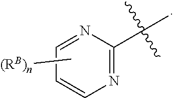

In some embodiments, Ring B is

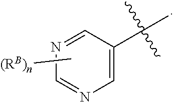

In some embodiments, Ring B is

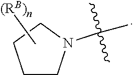

In some embodiments, Ring B is

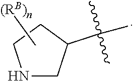

In some embodiments, Ring B is

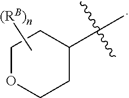

In some embodiments, Ring B is

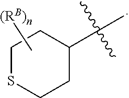

In some embodiments, Ring B is

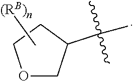

In some embodiments, Ring B is

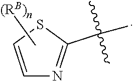

In some embodiments, Ring B is

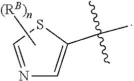

In some embodiments, Ring B is

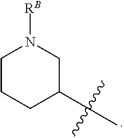

In some embodiments, Ring B is

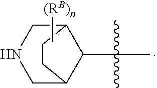

In some embodiments, Ring B is

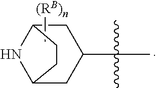

In some embodiments, Ring B is

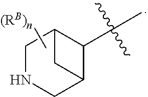

In some embodiments, Ring B is

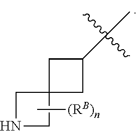

In some embodiments, Ring B is

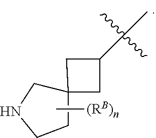

In some embodiments, Ring B is

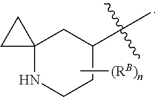

In some embodiments, Ring B is

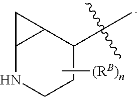

In some embodiments of a compound of Formula (I), each R^{B} is independently selected from the group consisting of halogen; —OH; oxo; cyano; —O(C₁-C₆ alkyl) optionally substituted with phenyl or 1 to 3 independently selected halogens; —C(O)(C₁-C₆ alkyl);
—C(O)O(C₁-C₆ alkyl); phenyl; 5- to 6-membered heteroaryl; 4- to 8-membered heterocycloalkyl;
3- to 8-membered cycloalkyl; —C(O)(3- to 8-membered cycloalkyl);
—C(O)(4-8-membered heterocycloalkyl); —S(O)₂(C₁-C₆ alkyl); —S(O)₂(3- to 8-membered cycloalkyl); —C(O)NR^{B1}R^{B2}; —S(O)₂NR^{B1}R^{B2}; —NR^{C1}S(O)₂NR^{B1}R^{B2}; —(C═N—R^{C1})—NR^{B1}R^{B2};
NR^{C1}—(C═N—R^{C1})—NR^{B1}R^{B2}; —NR^{C1}—(C═N—CN)—NR^{B1}R^{B2}; and C₁-C₆ alkyl optionally substituted with 1 to 5 substituents independently selected from the group consisting of halogen, —OH, and —O(C₁-C₆ alkyl); wherein R^{B1} and R^{B2} are each independently hydrogen or C₁-C₆ alkyl.

In some embodiments of a compound of Formula (I), each R^{B} is independently selected from the group consisting of halogen; oxo; —O(C₁-C₆ alkyl) optionally substituted with phenyl or 1 to 3 independently selected halogens; C₁-C₆ alkyl optionally substituted with 1 to 5 substituents independently selected from halogen, —OH, 4- to 8-membered heterocycloalkyl, and —O(C₁-C₆ alkyl); —S(O)₂(C₁-C₆ alkyl); —S(O)₂(4- to 8-membered heterocycloalkyl); —S(O)₂(3- to 8-membered cycloalkyl); —S(O)₂NR^{B1}R^{B2}; —C(O)NR^{B1}R^{B2}; —C(O)(C₁-C₆ alkyl); —C(O)O(C₁-C₆ alkyl); —C(O)(3- to 8-membered cycloalkyl) optionally substituted with 1 to 3 independently selected halogens; —C(O)(4- to 8-membered heterocycloalkyl) optionally substituted with 1 to 3 independently selected halogens; 4- to 8-membered heterocycloalkyl; and 5- to 6-membered heteroaryl. In some embodiments of a compound of Formula (I), each R^{B} is independently selected from the group consisting of halogen; oxo; —O(C₁-C₆ alkyl) optionally substituted with phenyl or 1 to 3 independently selected halogens; C₁-C₆ alkyl optionally substituted with 1 to 5 substituents independently selected from halogen, —OH, and —O(C₁-C₆ alkyl); —S(O)₂(C₁-C₆ alkyl); —S(O)₂NR^{B1}R^{B2}; —C(O)NR^{B1}R^{B2}; —C(O)(C₁-C₆ alkyl); 4- to 8-membered heterocycloalkyl; and 5- to 6-membered heteroaryl. In some embodiments of a compound of Formula (I), each R^{B} is independently selected from the group consisting of —O(C₁-C₄ alkyl) optionally substituted with phenyl or 1 to 3 independently selected halogens; and C₁-C₄ alkyl optionally substituted with 1 to 5 substituents independently selected from halogen, —OH, 4- to 8-membered heterocycloalkyl, and —O(C₁-C₄ alkyl). In some embodiments, each R^{B} is independently selected from the group consisting of chloro, fluoro, or bromo; oxo; unsubstituted —O(C₁-C₃ alkyl); —O(C₁-C₃ alkyl) substituted with phenyl; —O(C₁-C₃ alkyl) substituted with 1 to 3 fluoro; —S(O)₂(C₁-C₃ alkyl); —S(O)₂NR^{B1}R^{B2}; unsubstituted C₁-C₃ alkyl; C₁-C₃ alkyl substituted with 1 to 5 substituents independently selected from chloro, fluoro, —OH, and —O(C₁-C₃ alkyl); —C(O)NH₂; C(O)NMe₂; —C(O)(C₁-C₃ alkyl); 4- to 6-membered heterocycloalkyl; and 6-membered heteroaryl. In some embodiments, each R^{B} is independently selected from the group consisting of chloro; fluoro; oxo; —OCH₃ optionally substituted with 1 to 3 fluoro; —CH₃; —C(O)NH₂; C(O)NMe₂; —C(O)(C₁-C₃ alkyl); 4- to 6-membered heterocycloalkyl; and 6-membered heteroaryl. In some embodiments, each R^{B} is independently selected from the group consisting of oxo, —C(O)(C₁-C₆ alkyl), —C(O)NR^{B1}R^{B2}, —S(O)₂(C₁-C₆ alkyl), —S(O)₂NR^{B1}R^{B2}, unsubstituted C₁-C₆ alkyl, 4- to 8-membered heterocycloalkyl, and 5- to 6-membered heteroaryl. In some embodiments, each R^{B} is independently selected from the group consisting of oxo, —C(O)(C₁-C₃ alkyl), —C(O)N(Me)₂, —C(O)(4- to 8-membered heterocycloalkyl) optionally substituted with 1 to 3 independently selected halogens, —C(O)(3- to 8-membered cycloalkyl) optionally substituted with 1 to 3 independently selected halogens; —S(O)₂(C₁-C₃ alkyl), —S(O)₂N(Me)₂, —C(O)O(C₁-C₄ alkyl), —S(O)₂(4- to 8-membered heterocycloalkyl), —S(O)₂(3- to 4-membered cycloalkyl), methyl, oxetanyl, and pyridinyl. In some embodiments, each R^{B} is independently selected from the group consisting of oxo, —C(O)(C₁-C₃ alkyl), —C(O)N(Me)₂, —S(O)₂Me, —S(O)₂N(Me)₂, methyl, oxetanyl, and pyridinyl. In some embodiments, R^{B} is oxo. In some embodiments, R^{B} is —S(O)₂NR^{B1}R^{B2}. In some embodiments, R^{B} is —S(O)₂(C₁-C₃ alkyl). In some embodiments, R^{B} is —S(O)₂(4- to 6-membered heterocycloalkyl). In some embodiments, R^{B} is —C(O)O(C₁-C₃ alkyl). In some embodiments, R^{B} is —C(O)(C₁-C₃ alkyl). In some embodiments, R^{B} is —C(O)(C₃ alkyl). In some embodiments, R^{B} is —C(O)(C₂ alkyl). In some embodiments, R^{B} is —C(O)(Me). In some embodiments, R^{B} is —C(O)N(Me)₂. In some embodiments, R^{B} is methyl. In some embodiments, R^{B} is oxetanyl. In some embodiments, R^{B} is pyridinyl. In some embodiments, R^{B} is —S(O)₂N(Me)₂. In some embodiments, R^{B} is —S(O)₂Et. In some embodiments, R^{B} is —S(O)₂Me.

In some embodiments of a compound of Formula (I), each R^{B} is independently selected from the group consisting of halogen; oxo; —O(C₁-C₆ alkyl) optionally substituted with phenyl or 1 to 3 independently selected halogens; C₁-C₆ alkyl optionally substituted with 1 to 5 substituents independently selected from halogen, —OH, and —O(C₁-C₆ alkyl); —S(O)₂(C₁-C₆ alkyl); —S(O)₂NR^{B1}R^{B2}; —C(O)NR^{B1}R^{B2}; —C(O)(C₁-C₆ alkyl); 4- to 8-membered heterocycloalkyl; and 5- to 6-membered heteroaryl. In some embodiments of a compound of Formula (I), each R^{B} is independently selected from the group consisting of halogen; oxo; —O(C₁-C₆ alkyl) optionally substituted with phenyl or 1 to 3 independently selected halogens; C₁-C₆alkyl optionally substituted with 1 to 5 substituents independently selected from halogen, —OH, and —O(C₁-C₆ alkyl); —S(O)₂(C₁-C₆ alkyl); —S(O)₂NR^{B1}R^{B2}; —C(O)NR^{B1}R^{B2}; —C(O)(C₁-C₆ alkyl); 4- to 8-membered heterocycloalkyl; and 5- to 6-membered heteroaryl. In some embodiments, each R^{B} is independently selected from the group consisting of chloro, fluoro, or bromo; oxo; unsubstituted —O(C₁-C₃ alkyl); —O(C₁-C₃ alkyl) substituted with phenyl; —O(C₁-C₃ alkyl) substituted with 1 to 3 fluoro; —S(O)₂(C₁-C₃ alkyl); —S(O)₂NR^{B1}R^{B2}; unsubstituted C₁-C₃ alkyl; C₁-C₃ alkyl substituted with 1 to 5 substituents independently selected from chloro, fluoro, —OH, and —O(C₁-C₃ alkyl);
—C(O)NH₂; C(O)NMe₂; —C(O)(C₁-C₃ alkyl); 4- to 6-membered heterocycloalkyl; and 6-membered heteroaryl. In some embodiments, each R^{B} is independently selected from the group consisting of chloro; fluoro; oxo; —OCH₃ optionally substituted with 1 to 3 fluoro; —CH₃; —C(O)NH₂; C(O)NMe₂; —C(O)(C₁-C₃ alkyl); 4- to 6-membered heterocycloalkyl; and 6-membered heteroaryl. In some embodiments, each R^{B} is independently selected from the group consisting of oxo, —C(O)(C₁-C₆ alkyl), —C(O)NR^{B1}R^{B2}, —S(O)₂(C₁-C₆ alkyl), —S(O)₂NR^{B1}R^{B2}, unsubstituted C₁-C₆ alkyl, 4- to 8-membered heterocycloalkyl, and 5- to 6-membered heteroaryl. In some embodiments, each R^{B} is independently selected from the group consisting of oxo, —C(O)(C₁-C₃ alkyl), —C(O)N(Me)₂, —S(O)₂Me, —S(O)₂N(Me)₂, methyl, oxetanyl, and pyridinyl. In some embodiments, R^{B} is oxo. In some embodiments, R^{B} is-S(O)₂NR^{B1}R^{B2}. In some embodiments, R^{B} is —S(O)₂(C₁-C₃ alkyl). In some embodiments, R^{B} is —C(O)(C₁-C₃ alkyl). In some embodiments, R^{B} is —C(O)N(Me)₂. In some embodiments, R^{B} is methyl. In some embodiments, R^{B} is oxetanyl. In some embodiments, R^{B} is pyridinyl. In some embodiments, R^{B} is —S(O)₂N(Me)₂. In some embodiments, R^{B} is —S(O)₂Me.

In some embodiments, R^{B1} and R^{B2} are each independently hydrogen or C₁-C₆ alkyl. In some embodiments, R^{B1} and R^{B2} are each hydrogen. In some embodiments, R^{B1} and R^{B2} are each C₁-C₆ alkyl. In some embodiments, R^{B1} is hydrogen. In some embodiments, R^{B1} is C₁-C₆ alkyl. In some embodiments, R^{B2} is hydrogen. In some embodiments, R^{B2} is C₁-C₆ alkyl.

In some embodiments, R^{C1} is hydrogen or C₁-C₆ alkyl. In some embodiments, R^{C1} is hydrogen. In some embodiments, R^{C1} is C₁-C₆ alkyl. In some embodiments, R^{C1} is methyl. In some embodiments, R^{C1} is ethyl.

In some embodiments of a compound of Formula (I), Ring B and R^{B} are taken together to form a moiety selected from the group consisting of:

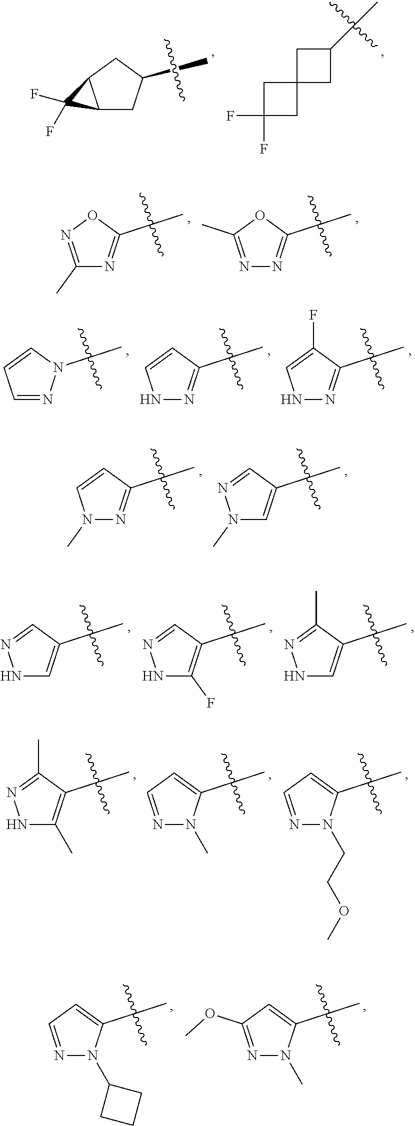

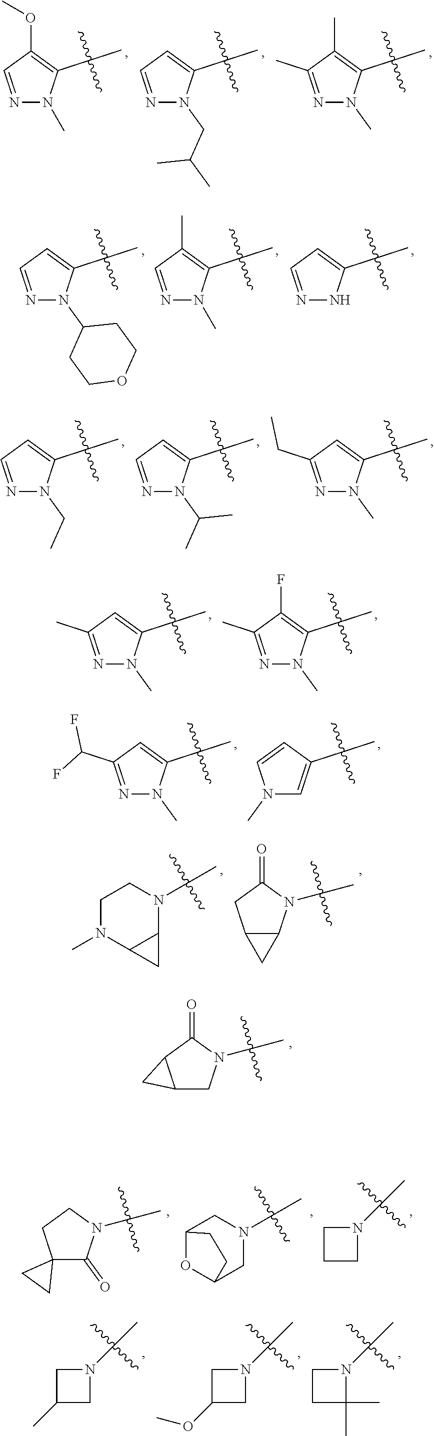

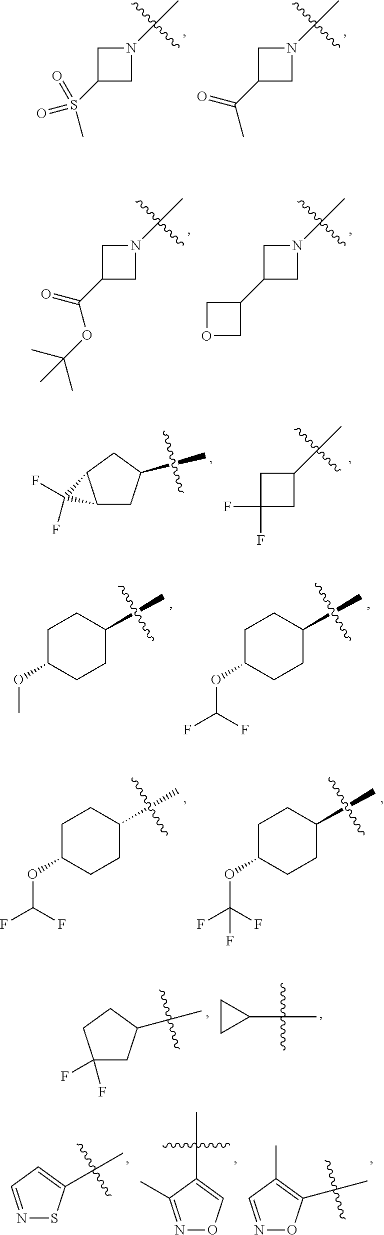

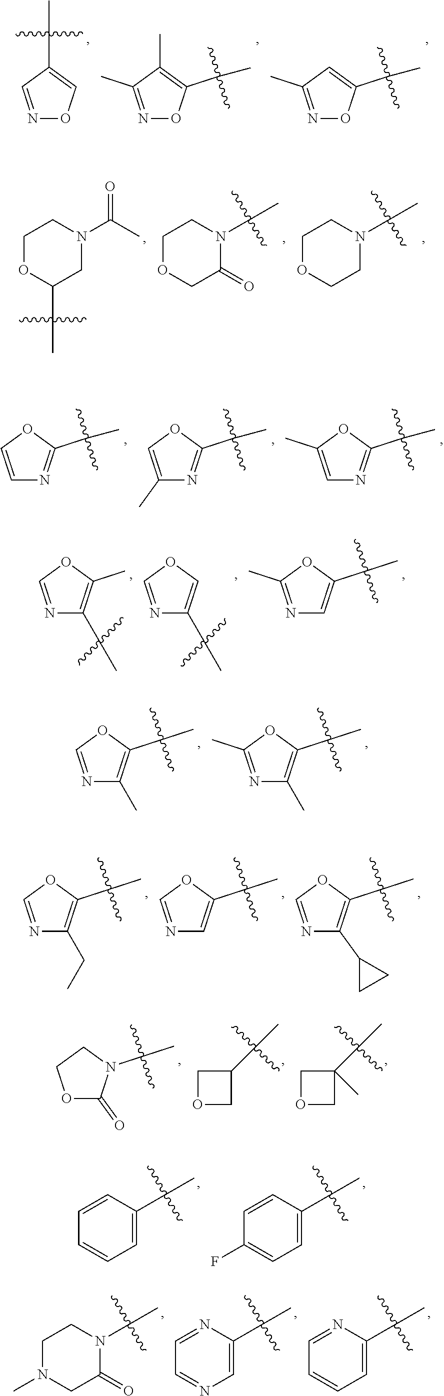

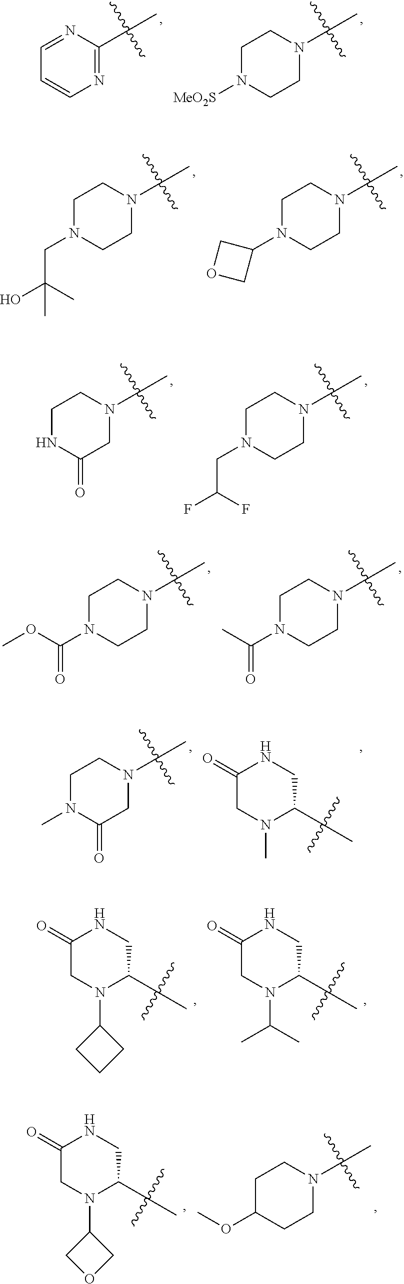

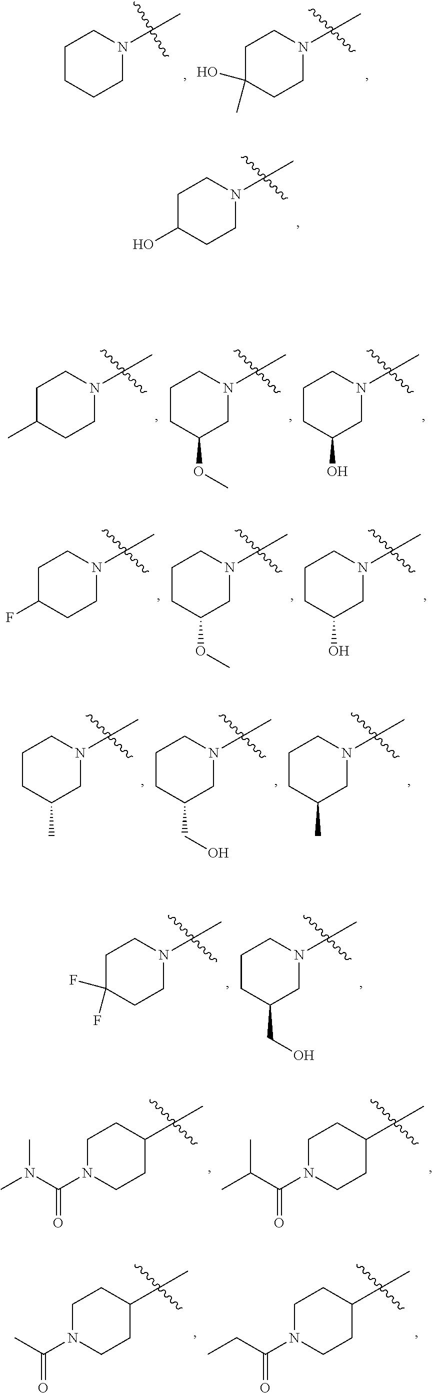

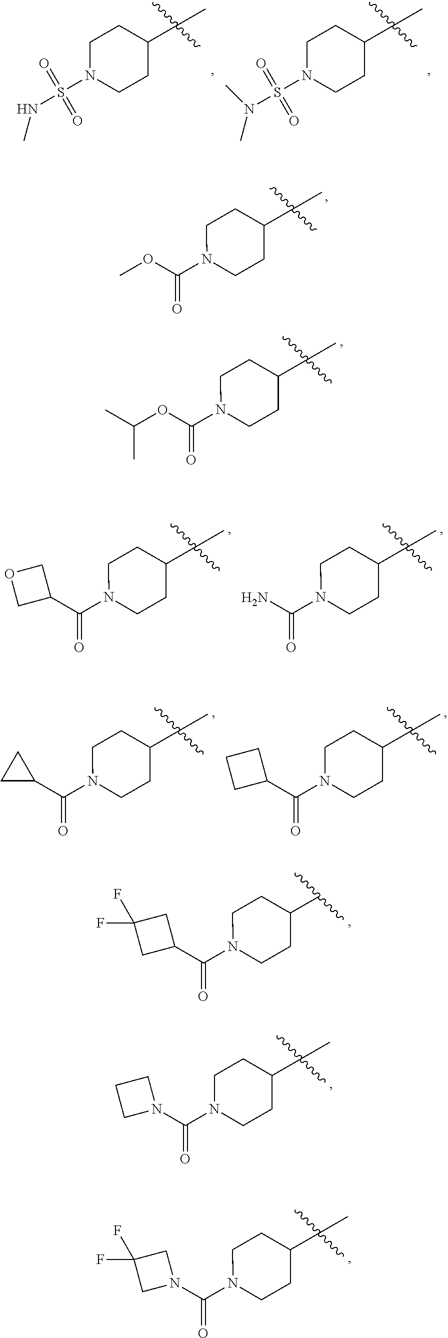

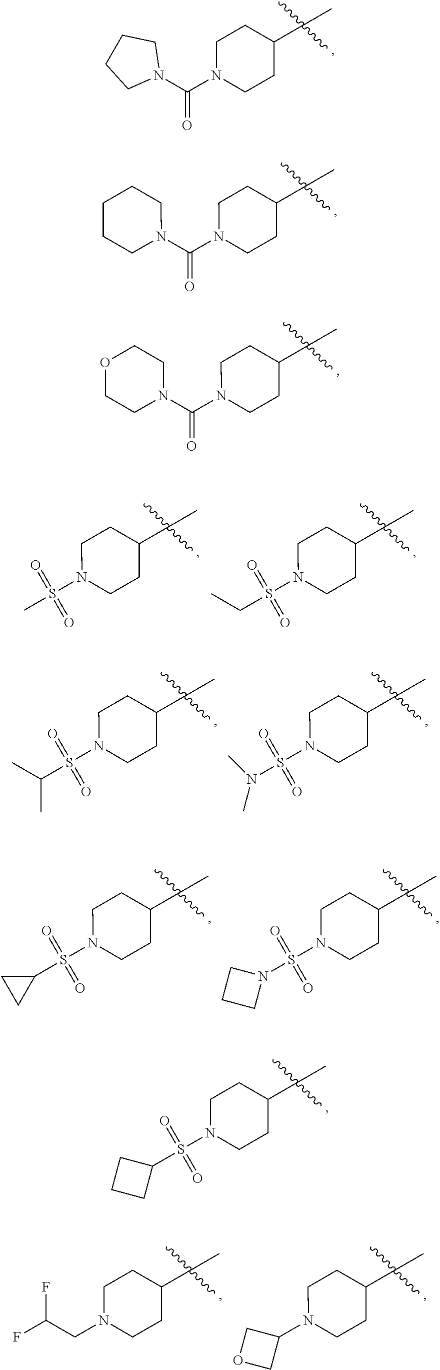

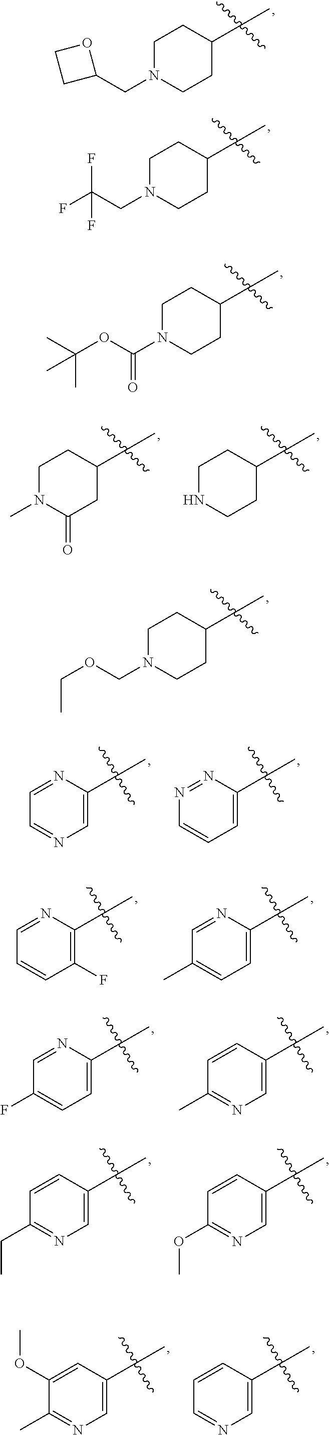

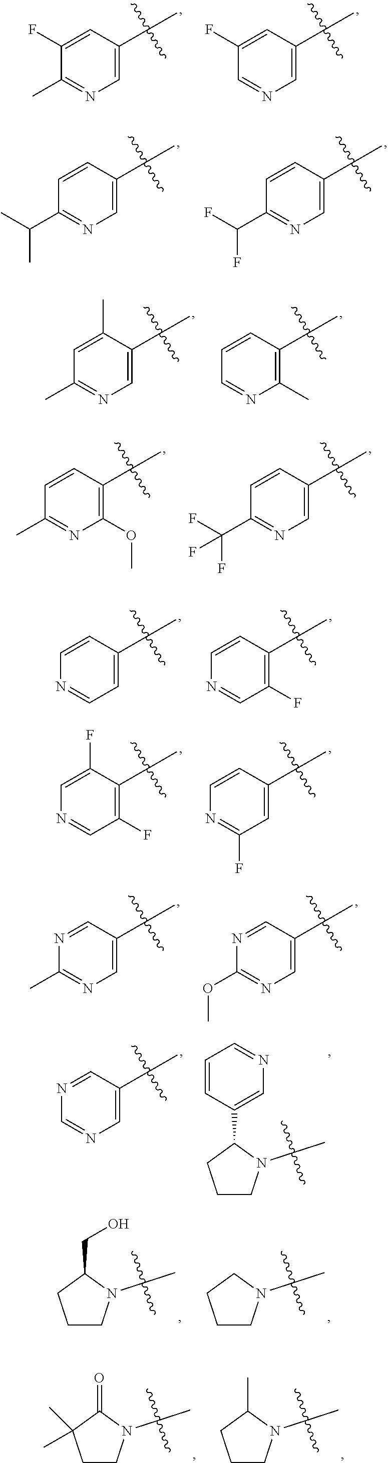

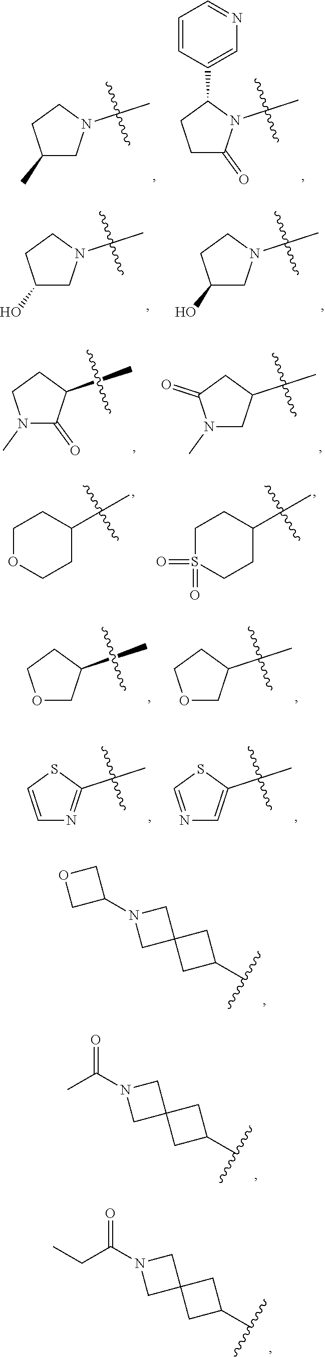

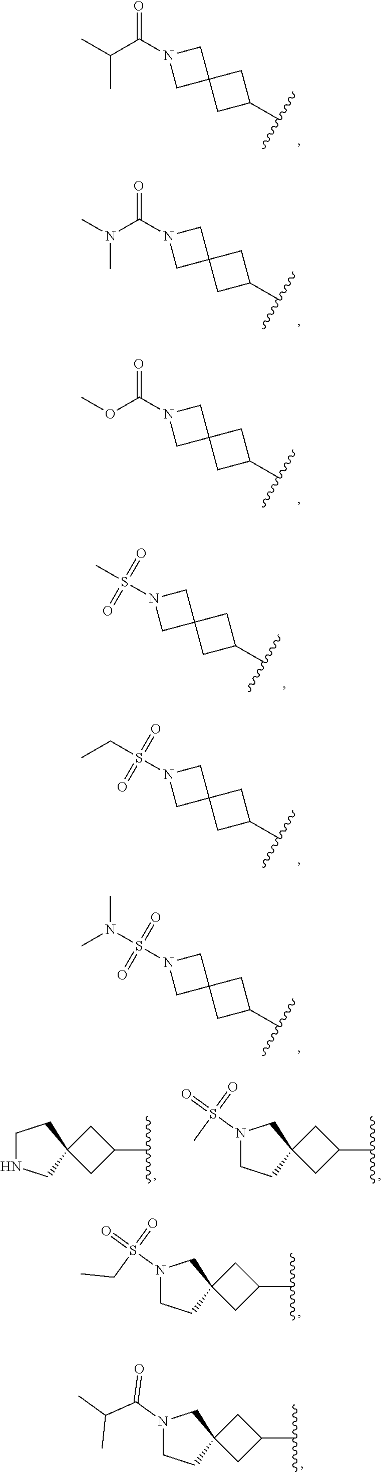

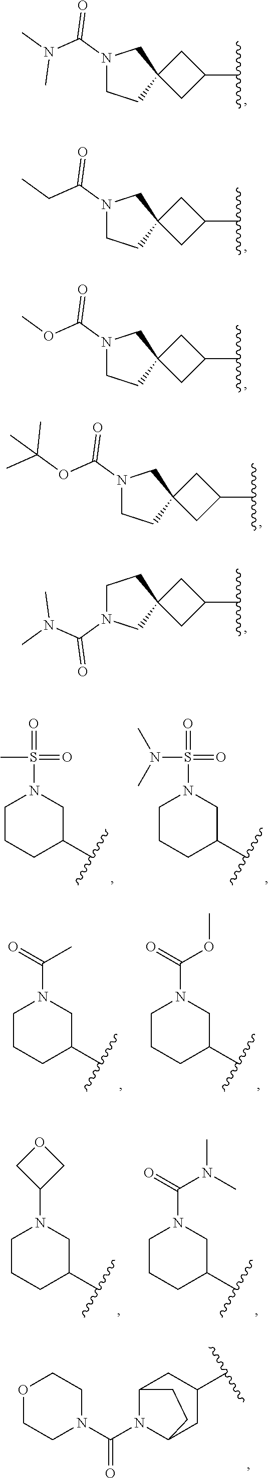

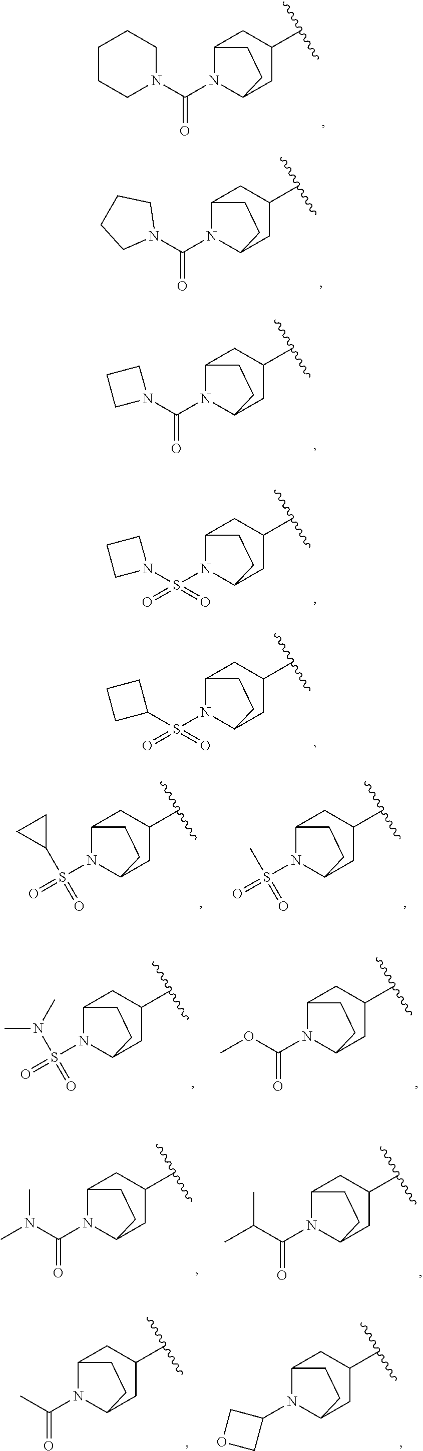

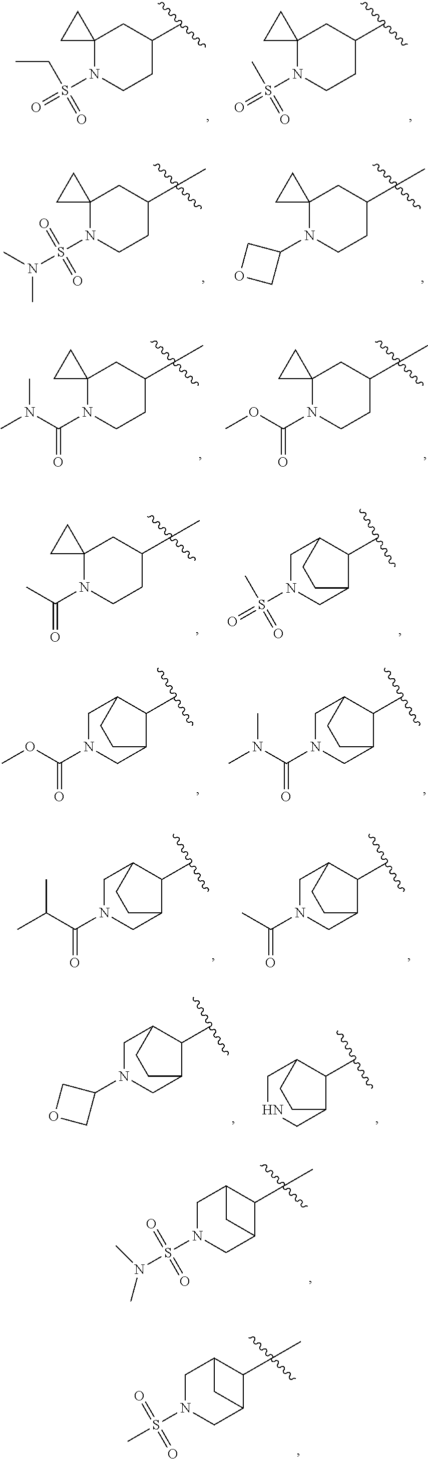

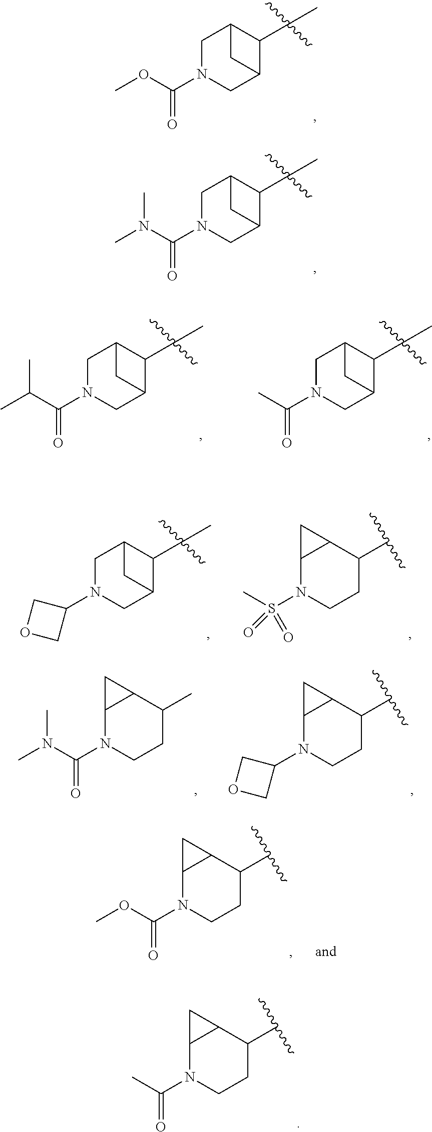

In some embodiments of a compound of Formula (I), Ring B and R^{B} are taken together to form a moiety selected from the group consisting of:

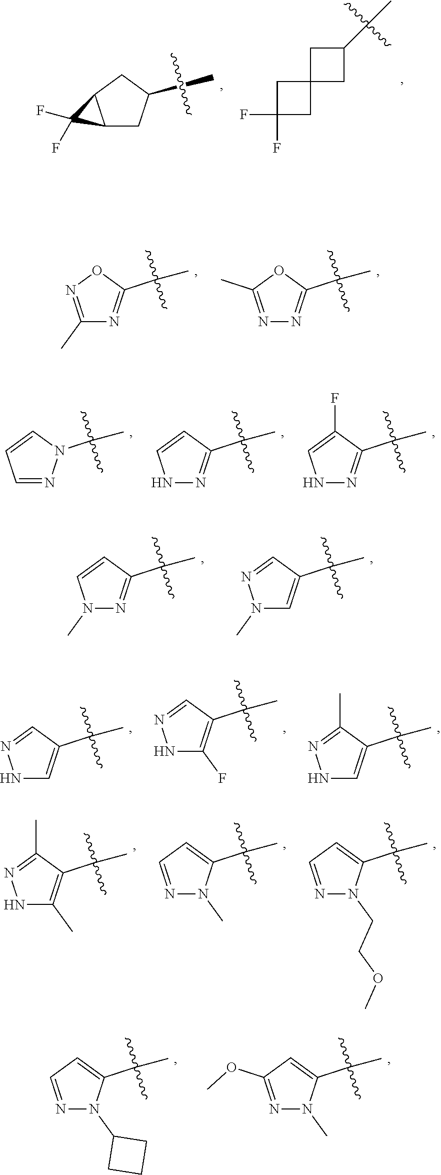

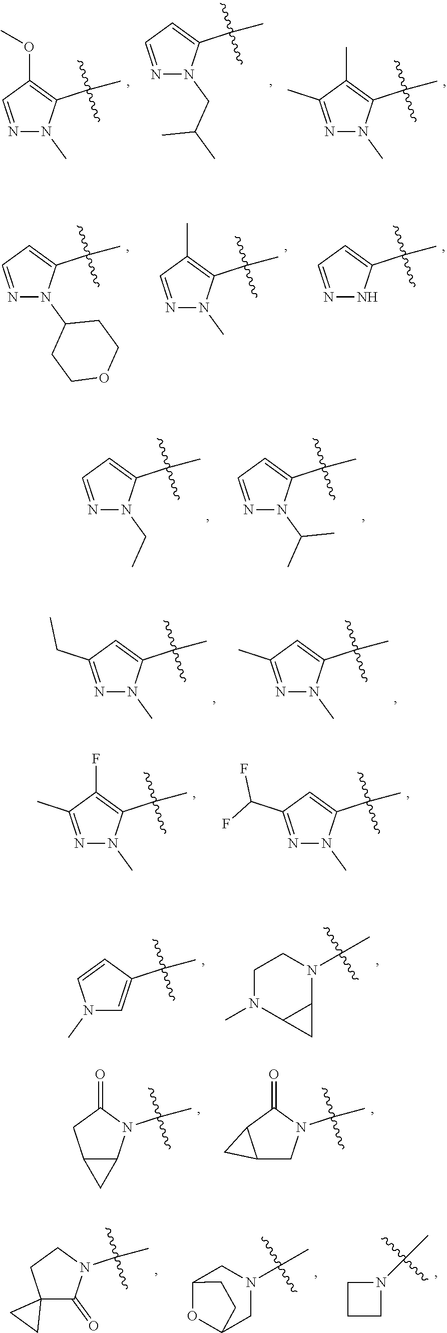

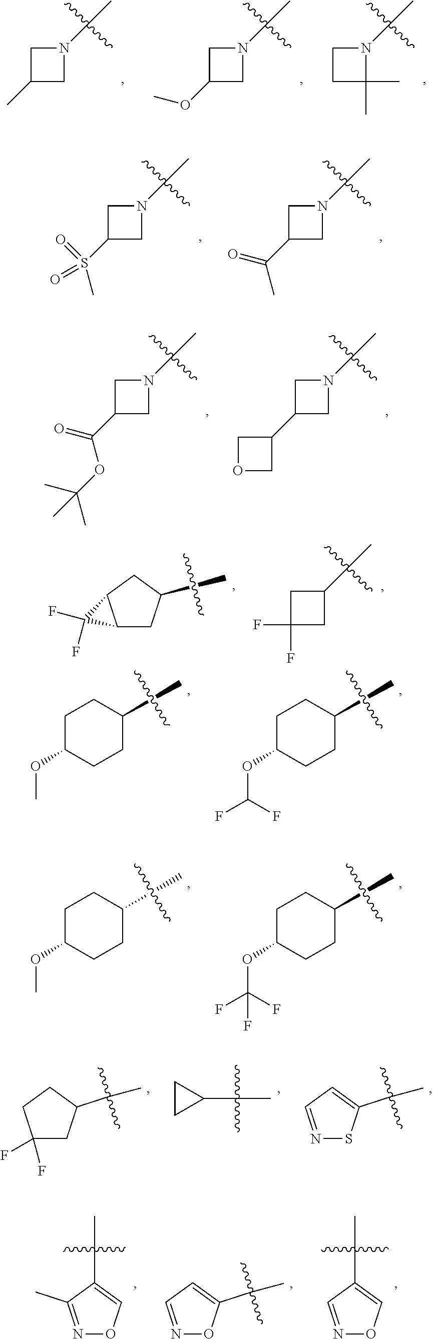

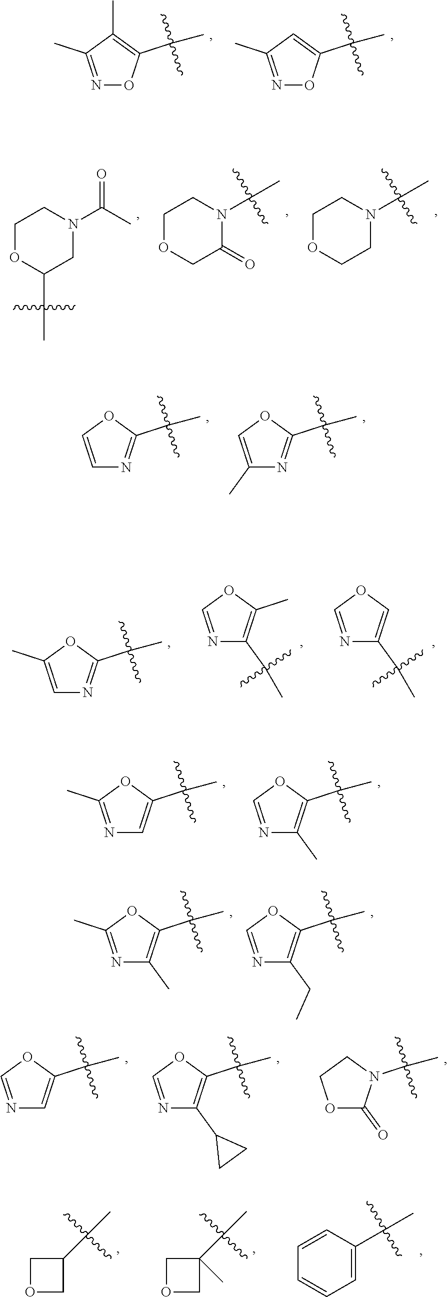

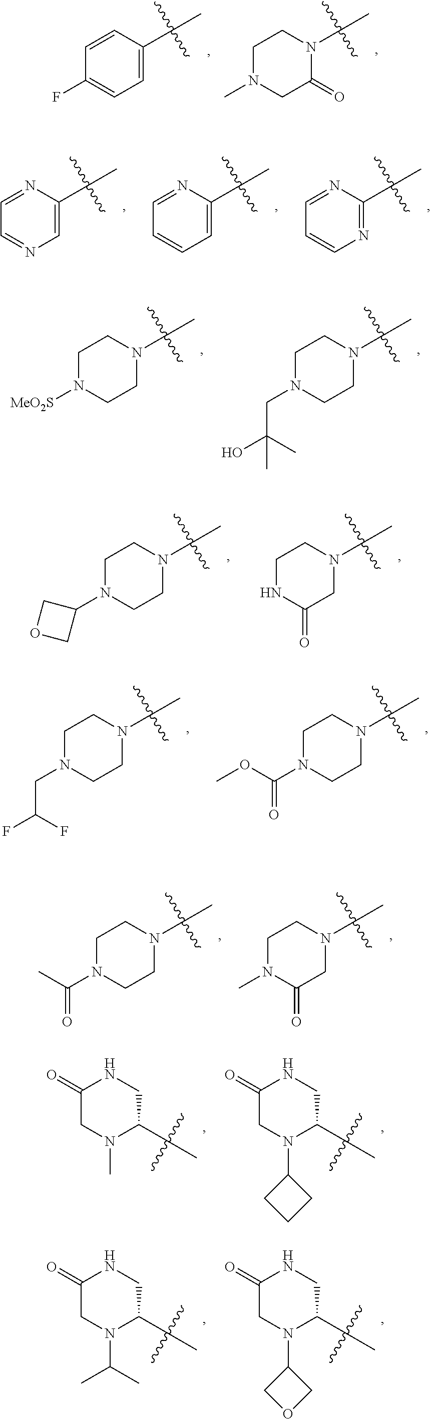

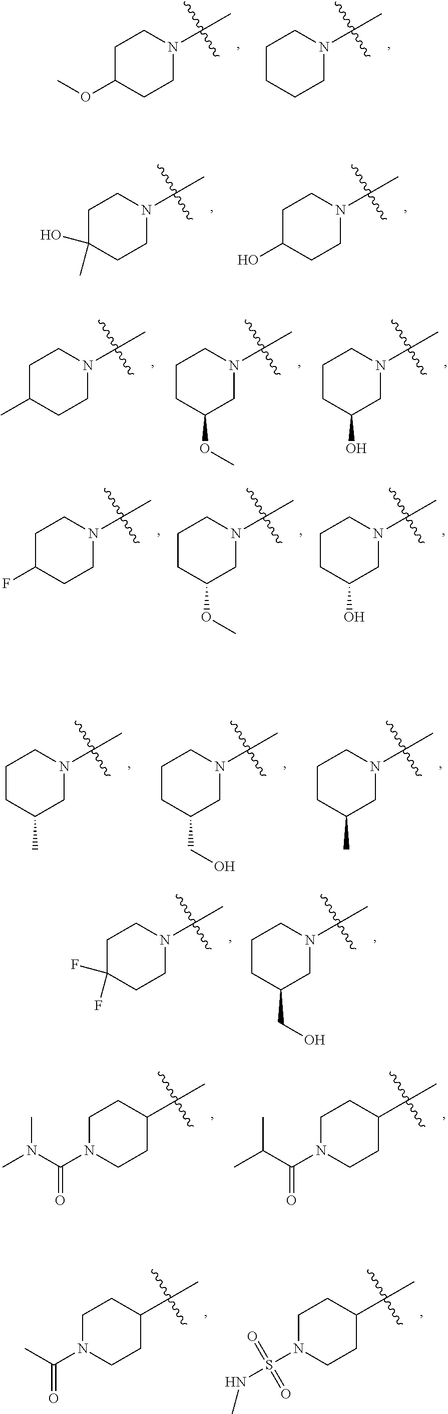

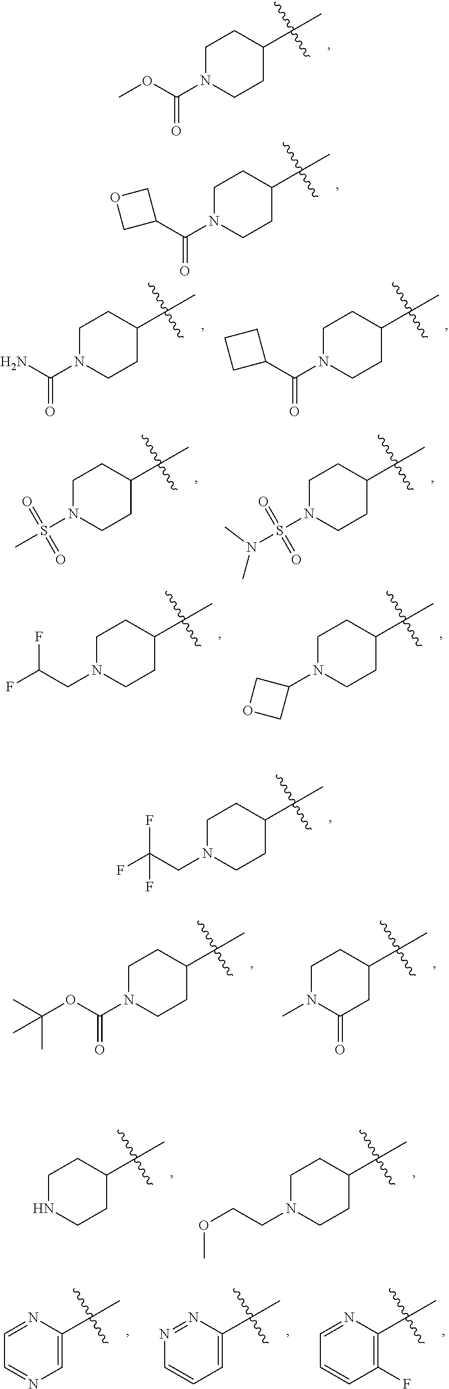

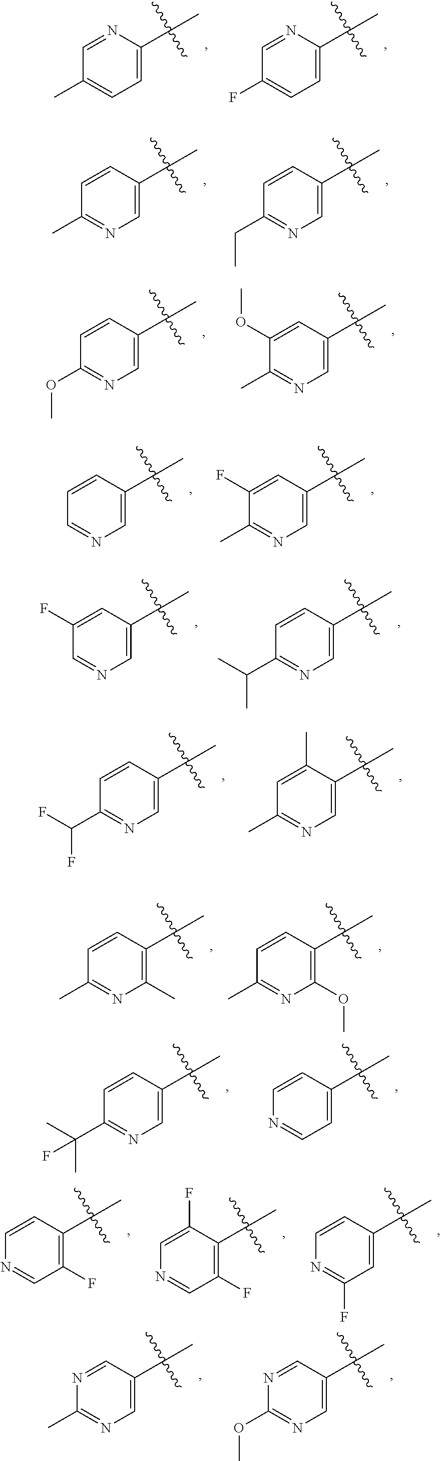

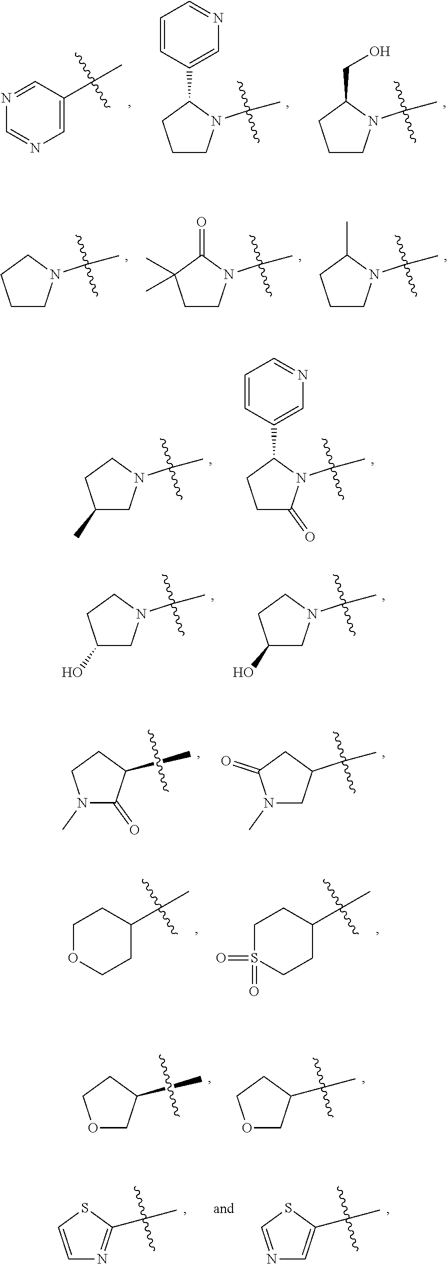

In some embodiments of a compound of Formula (I), Ring B and R^{B} are taken together to form

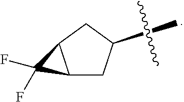

In some embodiments of a compound of Formula (I), Ring B and R^{B} are taken together to form

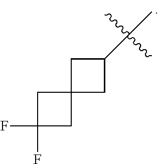 [SMILES: *C1CC2(C1)CC(F)(F)C2]

In some embodiments of a compound of Formula (I), Ring B and R^{B} are taken together to form

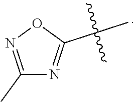 [SMILES: *c1nc(C)no1]

In some embodiments of a compound of Formula (I), Ring B and R^{B} are taken together to form

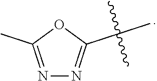 [SMILES: *c1nnc(C)o1]

In some embodiments of a compound of Formula (I), Ring B and R^{B} are taken together to form

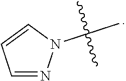 [SMILES: *n1cccn1]

In some embodiments of a compound of Formula (I), Ring B and R^{B} are taken together to form

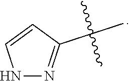 [SMILES: *c1cc[nH]n1]

In some embodiments of a compound of Formula (I), Ring B and R^{B} are taken together to form

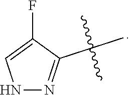 [SMILES: *c1n[nH]cc1F]

In some embodiments of a compound of Formula (I), Ring B and R^{B} are taken together to form

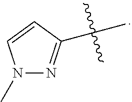 [SMILES: *c1ccn(C)n1]

In some embodiments of a compound of Formula (I), Ring B and R^{B} are taken together to form

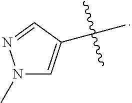 [SMILES: *c1cnn(C)c1]

In some embodiments of a compound of Formula (I), Ring B and R^{B} are taken together to form

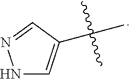 [SMILES: *c1cn[nH]c1]

In some embodiments of a compound of Formula (I), Ring B and R^{B} are taken together to form

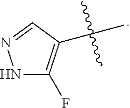 [SMILES: *c1cn[nH]c1F]

In some embodiments of a compound of Formula (I), Ring B and R^{B} are taken together to form

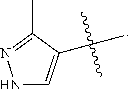 [SMILES: *c1c[nH]nc1C]

In some embodiments of a compound of Formula (I), Ring B and R^{B} are taken together to form

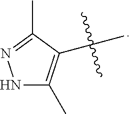 [SMILES: *c1c(C)n[nH]c1C]

In some embodiments of a compound of Formula (I), Ring B and R^{B} are taken together to form

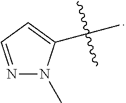 [SMILES: *c1ccnn1C]

In some embodiments of a compound of Formula (I), Ring B and R^{B} are taken together to form

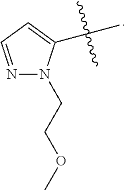 [SMILES: *c1ccnn1CCOC]

In some embodiments of a compound of Formula (I), Ring B and R^{B} are taken together to form

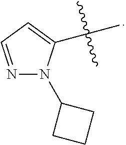 [SMILES: *c1ccnn1C1CCC1]

In some embodiments of a compound of Formula (I), Ring B and R^{B} are taken together to form

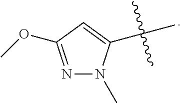 [SMILES: *c1cc(OC)nn1C]

In some embodiments of a compound of Formula (I), Ring B and R^{B} are taken together to form

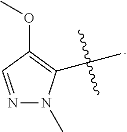 [SMILES: *c1c(OC)cnn1C]

In some embodiments of a compound of Formula (I), Ring B and R^{B} are taken together to form

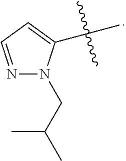 [SMILES: *c1ccnn1CC(C)C]

In some embodiments of a compound of Formula (I), Ring B and R^{B} are taken together to form

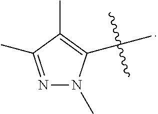 [SMILES: *c1c(C)c(C)nn1C]

In some embodiments of a compound of Formula (I), Ring B and R^{B} are taken together to form

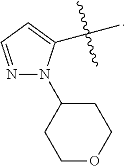 [SMILES: *c1ccnn1C1CCOCC1]

In some embodiments of a compound of Formula (I), Ring B and R^{B} are taken together to form

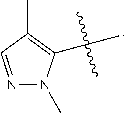 [SMILES: *c1c(C)cnn1C]

In some embodiments of a compound of Formula (I), Ring B and R^{B} are taken together to form

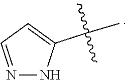 [SMILES: *c1ccn[nH]1]

In some embodiments of a compound of Formula (I), Ring B and R^{B} are taken together to form

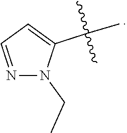 [SMILES: *c1ccnn1CC]

In some embodiments of a compound of Formula (I), Ring B and R^{B} are taken together to form

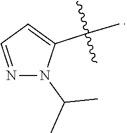 [SMILES: *c1ccnn1C(C)C]

In some embodiments of a compound of Formula (I), Ring B and R^{B} are taken together to form

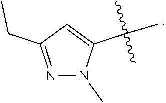 [SMILES: *c1cc(CC)nn1C]

In some embodiments of a compound of Formula (I), Ring B and R^{B} are taken together to form

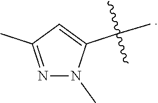 [SMILES: *c1cc(C)nn1C]

In some embodiments of a compound of Formula (I), Ring B and R^{B} are taken together to form

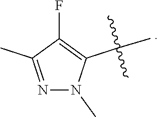 [SMILES: *c1c(F)c(C)nn1C]

In some embodiments of a compound of Formula (I), Ring B and R^{B} are taken together to form

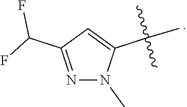 [SMILES: *c1cc(C(F)F)nn1C]

In some embodiments of a compound of Formula (I), Ring B and R^{B} are taken together to form

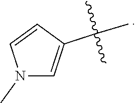 [SMILES: *c1ccn(C)c1]

In some embodiments of a compound of Formula (I), Ring B and R^{B} are taken together to form

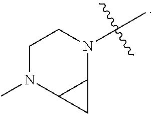 [SMILES: *N1CCN(C)C2CC21]

In some embodiments of a compound of Formula (I), Ring B and R^{B} are taken together to form

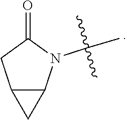 [SMILES: *N1C(=O)CC2CC21]

In some embodiments of a compound of Formula (I), Ring B and R^{B} are taken together to form

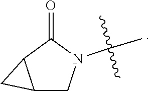 [SMILES: *N1CC2CC2C1=O]

In some embodiments of a compound of Formula (I), Ring B and R^{B} are taken together to form

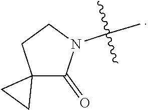 [SMILES: *N1CCC2(CC2)C1=O]

In some embodiments of a compound of Formula (I), Ring B and R^{B} are taken together to form

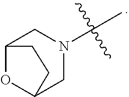 [SMILES: *N1CC2CCC(C1)O2]

In some embodiments of a compound of Formula (I), Ring B and R^{B} are taken together to form

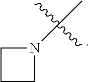 [SMILES: *N1CCC1]

In some embodiments of a compound of Formula (I), Ring B and R^{B} are taken together to form

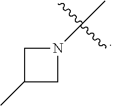 [SMILES: *N1CC(C)C1]

In some embodiments of a compound of Formula (I), Ring B and R^{B} are taken together to form

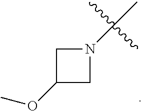 [SMILES: *N1CC(OC)C1]

In some embodiments of a compound of Formula (I), Ring B and R^{B} are taken together to form

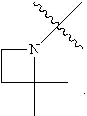 [SMILES: *N1CCC1(C)C]

In some embodiments of a compound of Formula (I), Ring B and R^{B} are taken together to form

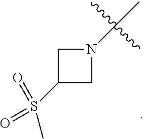 [SMILES: *N1CC(S(C)(=O)=O)C1]

In some embodiments of a compound of Formula (I), Ring B and R^{B} are taken together to form

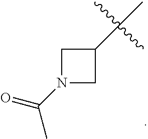 [SMILES: *C1CN(C(C)=O)C1]

In some embodiments of a compound of Formula (I), Ring B and R^{B} are taken together to form

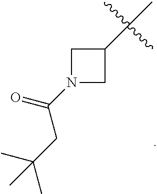 [SMILES: *C1CN(C(=O)CC(C)(C)C)C1]

In some embodiments of a compound of Formula (I), Ring B and R^{B} are taken together to form

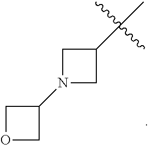 [SMILES: *C1CN(C2COC2)C1]

In some embodiments of a compound of Formula (I), Ring B and R^{B} are taken together to form

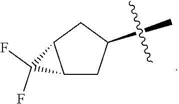

In some embodiments of a compound of Formula (I), Ring B and R^{B} are taken together to form

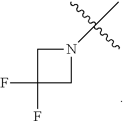 [SMILES: *N1CC(F)(F)C1]

In some embodiments of a compound of Formula (I), Ring B and R^{B} are taken together to form

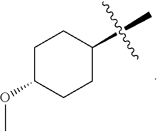

In some embodiments of a compound of Formula (I), Ring B and R^{B} are taken together to form

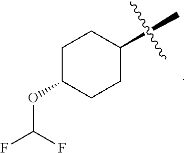

In some embodiments of a compound of Formula (I), Ring B and R^{B} are taken together to form

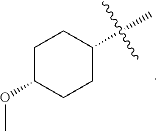

In some embodiments of a compound of Formula (I), Ring B and R^{B} are taken together to form

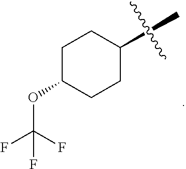

In some embodiments of a compound of Formula (I), Ring B and R^{B} are taken together to form

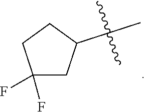 [SMILES: *C1CCC(F)(F)C1]

In some embodiments of a compound of Formula (I), Ring B and R^{B} are taken together to form

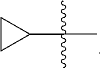 [SMILES: *C1CC1]

In some embodiments of a compound of Formula (I), Ring B and R^{B} are taken together to form

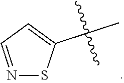 [SMILES: *c1ccns1]

In some embodiments of a compound of Formula (I), Ring B and R^{B} are taken together to form

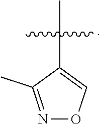 [SMILES: *c1conc1C]

In some embodiments of a compound of Formula (I), Ring B and R^{B} are taken together to form

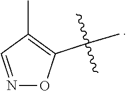 [SMILES: *c1oncc1C]

In some embodiments of a compound of Formula (I), Ring B and R^{B} are taken together to form

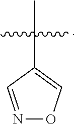 [SMILES: *c1cnoc1]

In some embodiments of a compound of Formula (I), Ring B and R^{B} are taken together to form

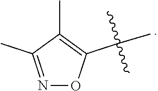 [SMILES: *c1onc(C)c1C]

In some embodiments of a compound of Formula (I), Ring B and R^{B} are taken together to form

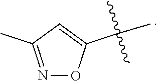 [SMILES: *c1cc(C)no1]

In some embodiments of a compound of Formula (I), Ring B and R^{B} are taken together to form

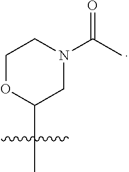 [SMILES: *C1CN(C(C)=O)CCO1]

In some embodiments of a compound of Formula (I), Ring B and R^{B} are taken together to form

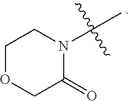 [SMILES: *N1CCOCC1=O]

In some embodiments of a compound of Formula (I), Ring B and R^{B} are taken together to form

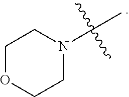 [SMILES: *N1CCOCC1]

In some embodiments of a compound of Formula (I), Ring B and R^{B} are taken together to form

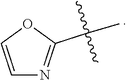 [SMILES: *c1ncco1]

In some embodiments of a compound of Formula (I), Ring B and R^{B} are taken together to form

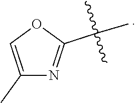 [SMILES: *c1nc(C)co1]

In some embodiments of a compound of Formula (I), Ring B and R^{B} are taken together to form

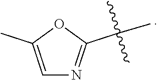 [SMILES: *c1ncc(C)o1]

In some embodiments of a compound of Formula (I), Ring B and R^{B} are taken together to form

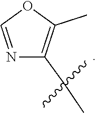 [SMILES: *c1ncoc1C]

In some embodiments of a compound of Formula (I), Ring B and R^{B} are taken together to form

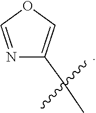 [SMILES: *c1cocn1]

In some embodiments of a compound of Formula (I), Ring B and R^{B} are taken together to form

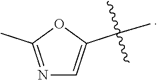 [SMILES: *c1cnc(C)o1]

In some embodiments of a compound of Formula (I), Ring B and R^{B} are taken together to form

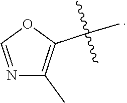 [SMILES: *c1ocnc1C]

In some embodiments of a compound of Formula (I), Ring B and R^{B} are taken together to form

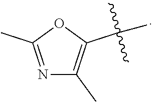

In some embodiments of a compound of Formula (I), Ring B and R^{B} are taken together to form

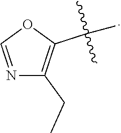

In some embodiments of a compound of Formula (I), Ring B and R^{B} are taken together to form

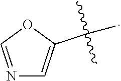

In some embodiments of a compound of Formula (I), Ring B and R^{B} are taken together to form

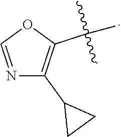

In some embodiments of a compound of Formula (I), Ring B and R^{B} are taken together to form

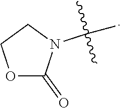

In some embodiments of a compound of Formula (I), Ring B and R^{B} are taken together to form

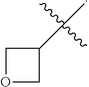

In some embodiments of a compound of Formula (I), Ring B and R^{B} are taken together to form

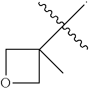

In some embodiments of a compound of Formula (I), Ring B and R^{B} are taken together to form

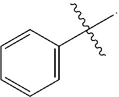

In some embodiments of a compound of Formula (I), Ring B and R^{B} are taken together to form

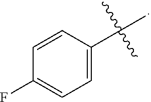

In some embodiments of a compound of Formula (I), Ring B and R^{B} are taken together to form

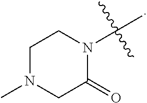

In some embodiments of a compound of Formula (I), Ring B and R^{B} are taken together to form

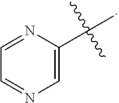

In some embodiments of a compound of Formula (I), Ring B and R^{B} are taken together to form

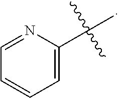

In some embodiments of a compound of Formula (I), Ring B and R^{B} are taken together to form

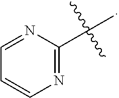

In some embodiments of a compound of Formula (I), Ring B and R^{B} are taken together to form

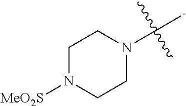

In some embodiments of a compound of Formula (I), Ring B and R^{B} taken together to form

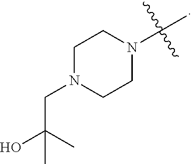

In some embodiments of a compound of Formula (I), Ring B and R^{B} are taken together to form

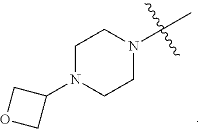 [SMILES: *N1CCN(C2COC2)CC1]

In some embodiments of a compound of Formula (I), Ring B and R^{B} are taken together to form

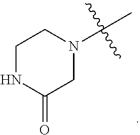 [SMILES: *N1CCNC(=O)C1]

In some embodiments of a compound of Formula (I), Ring B and R^{B} are taken together to form

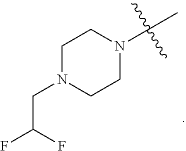 [SMILES: *N1CCN(CC(F)F)CC1]

In some embodiments of a compound of Formula (I), Ring B and R^{B} are taken together to form

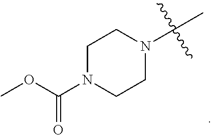 [SMILES: *N1CCN(C(=O)OC)CC1]

In some embodiments of a compound of Formula (I), Ring B and R^{B} are taken together to form

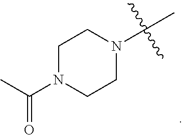 [SMILES: *N1CCN(C(C)=O)CC1]

In some embodiments of a compound of Formula (I), Ring B and R^{B} are taken together to form

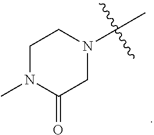 [SMILES: *N1CCN(C)C(=O)C1]

In some embodiments of a compound of Formula (I), Ring B and R^{B} are taken together to form

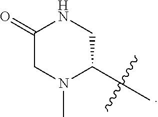 [SMILES: *[C@H]1CNC(=O)CN1C]

In some embodiments of a compound of Formula (I), Ring B and R^{B} are taken together to form

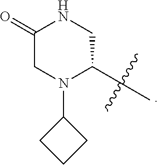 [SMILES: *[C@H]1CNC(=O)CN1C1CCC1]

In some embodiments of a compound of Formula (I), Ring B and R^{B} are taken together to form

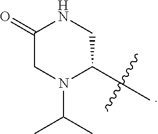 [SMILES: *[C@H]1CNC(=O)CN1C(C)C]

In some embodiments of a compound of Formula (I), Ring B and R^{B} are taken together to form

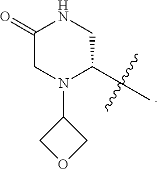 [SMILES: *[C@H]1CNC(=O)CN1C1COC1]

In some embodiments of a compound of Formula (I), Ring B and R^{B} are taken together to form

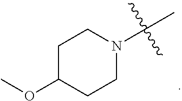 [SMILES: *N1CCC(OC)CC1]

In some embodiments of a compound of Formula (I), Ring B and R^{B} are taken together to form

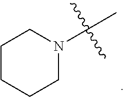 [SMILES: *N1CCCCC1]

In some embodiments of a compound of Formula (I), Ring B and R^{B} are taken together to form

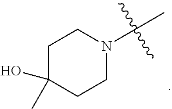 [SMILES: *N1CCC(C)(O)CC1]

In some embodiments of a compound of Formula (I), Ring B and R^{B} are taken together to form

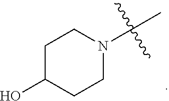 [SMILES: *N1CCC(O)CC1]

In some embodiments of a compound of Formula (I), Ring B and R^{B} are taken together to form

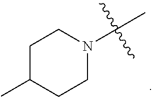 [SMILES: *N1CCC(C)CC1]

In some embodiments of a compound of Formula (I), Ring B and R^{B} are taken together to form

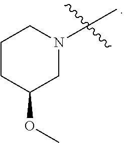 [SMILES: *N1CCC[C@H](OC)C1]

In some embodiments of a compound of Formula (I), Ring B and R^{B} are taken together to form

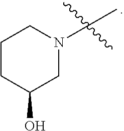 [SMILES: *N1CCC[C@H](O)C1]

In some embodiments of a compound of Formula (I), Ring B and R^{B} are taken together to form

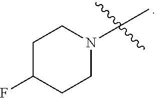 [SMILES: *N1CCC(F)CC1]

In some embodiments of a compound of Formula (I), Ring B and R^{B} are taken together to form

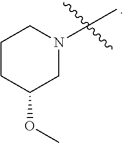 [SMILES: *N1CCC[C@@H](OC)C1]

In some embodiments of a compound of Formula (I), Ring B and R^{B} are taken together to form

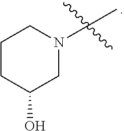 [SMILES: *N1CCC[C@@H](O)C1]

In some embodiments of a compound of Formula (I), Ring B and R^{B} are taken together to form

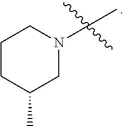 [SMILES: *N1CCC[C@@H](C)C1]

In some embodiments of a compound of Formula (I), Ring B and R^{B} are taken together to form

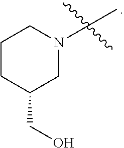 [SMILES: *N1CCC[C@@H](CO)C1]

In some embodiments of a compound of Formula (I), Ring B and R^{B} are taken together to form

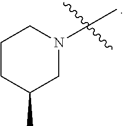 [SMILES: *N1CCC[C@H](C)C1]

In some embodiments of a compound of Formula (I), Ring B and R^{B} are taken together to form

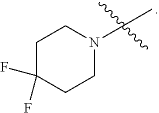 [SMILES: *N1CCC(F)(F)CC1]

In some embodiments of a compound of Formula (I), Ring B and R^{B} are taken together to form

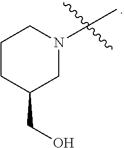 [SMILES: *N1CCC[C@H](CO)C1]

In some embodiments of a compound of Formula (I), Ring B and R^{B} are taken together to form

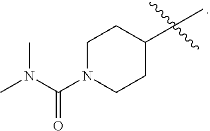 [SMILES: *C1CCN(C(=O)N(C)C)CC1]

In some embodiments of a compound of Formula (I), Ring B and R^{B} are taken together to form

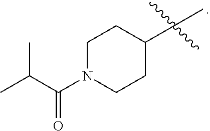 [SMILES: *C1CCN(C(=O)C(C)C)CC1]

In some embodiments of a compound of Formula (I), Ring B and R^{B} are taken together to form

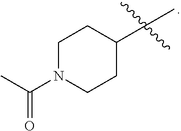 [SMILES: *C1CCN(C(C)=O)CC1]

In some embodiments of a compound of Formula (I), Ring B and R^{B} are taken together to form

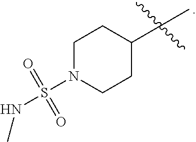 [SMILES: *C1CCN(S(=O)(=O)NC)CC1]

In some embodiments of a compound of Formula (I), Ring B and R^{B} are taken together to form

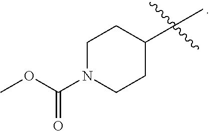 [SMILES: *C1CCN(C(=O)OC)CC1]

In some embodiments of a compound of Formula (I), Ring B and R^{B} are taken together to form

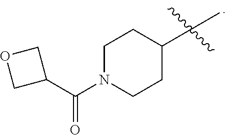 [SMILES: *C1CCN(C(=O)C2COC2)CC1]

In some embodiments of a compound of Formula (I), Ring B and R^{B} are taken together to form

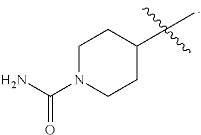 [SMILES: *C1CCN(C(N)=O)CC1]

In some embodiments of a compound of Formula (I), Ring B and R^{B} are taken together to form

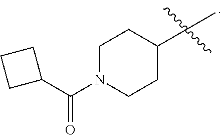 [SMILES: *C1CCN(C(=O)C2CCC2)CC1]

In some embodiments of a compound of Formula (I), Ring B and R^{B} are taken together to form

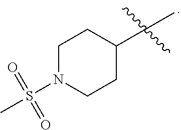 [SMILES: *C1CCN(S(C)(=O)=O)CC1]

In some embodiments of a compound of Formula (I), Ring B and R^{B} are taken together to form

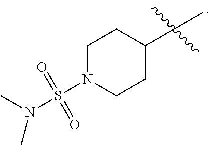 [SMILES: *C1CCN(S(=O)(=O)N(C)C)CC1]

In some embodiments of a compound of Formula (I), Ring B and R^{B} are taken together to form

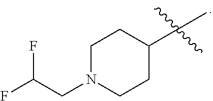 [SMILES: *C1CCN(CC(F)F)CC1]

In some embodiments of a compound of Formula (I), Ring B and R^{B} are taken together to form

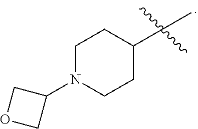 [SMILES: *C1CCN(C2COC2)CC1]

In some embodiments of a compound of Formula (I), Ring B and R^{B} are taken together to form

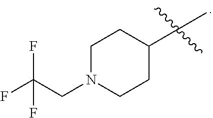 [SMILES: *C1CCN(CC(F)(F)F)CC1]

In some embodiments of a compound of Formula (I), Ring B and R^{B} are taken together to form

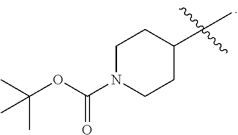 [SMILES: *C1CCN(C(=O)OC(C)(C)C)CC1]

In some embodiments of a compound of Formula (I), Ring B and R^{B} are taken together to form

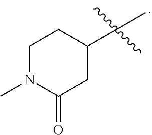 [SMILES: *C1CCN(C)C(=O)C1]

In some embodiments of a compound of Formula (I), Ring B and R^{B} are taken together to form

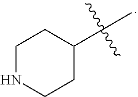 [SMILES: *C1CCNCC1]

In some embodiments of a compound of Formula (I), Ring B and R^{B} are taken together to form

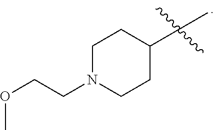 [SMILES: *C1CCN(CCOC)CC1]

In some embodiments of a compound of Formula (I), Ring B and R^{B} are taken together to form

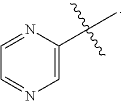 [SMILES: *c1cnccn1]

In some embodiments of a compound of Formula (I), Ring B and R^{B} are taken together to form

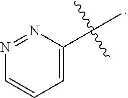 [SMILES: *c1cccnn1]

In some embodiments of a compound of Formula (I), Ring B and R^{B} are taken together to form

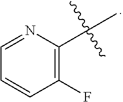 [SMILES: *c1ncccc1F]

In some embodiments of a compound of Formula (I), Ring B and R^{B} are taken together to form

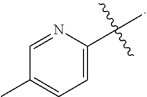

In some embodiments of a compound of Formula (I), Ring B and R^{B} are taken together to form

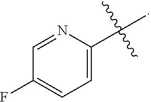

In some embodiments of a compound of Formula (I), Ring B and R^{B} are taken together to form

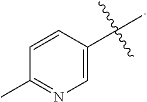

In some embodiments of a compound of Formula (I), Ring B and R^{B} are taken together to form

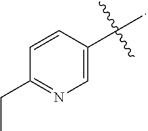

In some embodiments of a compound of Formula (I), Ring B and R^{B} are taken together to form

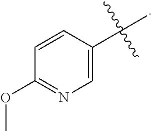

In some embodiments of a compound of Formula (I), Ring B and R^{B} are taken together to form

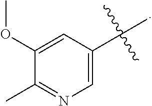

In some embodiments of a compound of Formula (I), Ring B and R^{B} are taken together to form

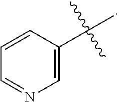

In some embodiments of a compound of Formula (I), Ring B and R^{B} are taken together to form

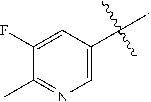

In some embodiments of a compound of Formula (I), Ring B and R^{B} are taken together to form

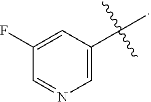

In some embodiments of a compound of Formula (I), Ring B and R^{B} are taken together to form

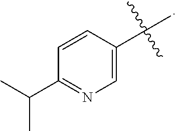

In some embodiments of a compound of Formula (I), Ring B and R^{B} are taken together to form

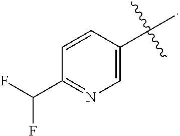

In some embodiments of a compound of Formula (I), Ring B and R^{B} are taken together to form

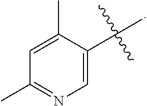

In some embodiments of a compound of Formula (I), Ring B and R^{B} are taken together to form

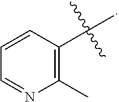

In some embodiments of a compound of Formula (I), Ring B and R^{B} are taken together to form

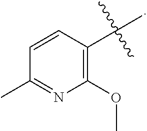

In some embodiments of a compound of Formula (I), Ring B and R^{B} are taken together to form

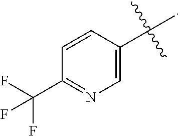

In some embodiments of a compound of Formula (I), Ring B and R^{B} are taken together to form

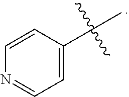 [SMILES: *c1ccncc1]

In some embodiments of a compound of Formula (I), Ring B and R^{B} are taken together to form

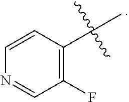 [SMILES: *c1ccncc1F]

In some embodiments of a compound of Formula (I), Ring B and R^{B} are taken together to form

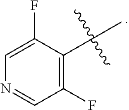 [SMILES: *c1c(F)cncc1F]

In some embodiments of a compound of Formula (I), Ring B and R^{B} are taken together to form

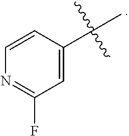 [SMILES: *c1ccnc(F)c1]

In some embodiments of a compound of Formula (I), Ring B and R^{B} are taken together to form

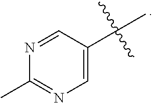 [SMILES: *c1cnc(C)nc1]

In some embodiments of a compound of Formula (I), Ring B and R^{B} are taken together to form

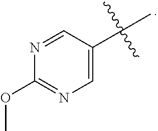 [SMILES: *c1cnc(OC)nc1]

In some embodiments of a compound of Formula (I), Ring B and R^{B} are taken together to form

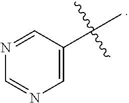 [SMILES: *c1cncnc1]

In some embodiments of a compound of Formula (I), Ring B and R^{B} are taken together to form

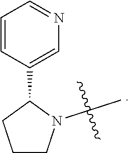 [SMILES: *N1CCC[C@@H]1c1cccnc1]

In some embodiments of a compound of Formula (I), Ring B and R^{B} are taken together to form

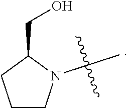 [SMILES: *N1CCC[C@H]1CO]

In some embodiments of a compound of Formula (I), Ring B and R^{B} are taken together to form

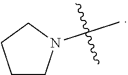 [SMILES: *N1CCCC1]

In some embodiments of a compound of Formula (I), Ring B and R^{B} are taken together to form

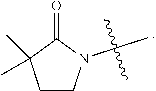 [SMILES: *N1CCC(C)(C)C1=O]

In some embodiments of a compound of Formula (I), Ring B and R^{B} are taken together to form

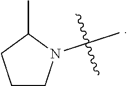 [SMILES: *N1CCCC1C]

In some embodiments of a compound of Formula (I), Ring B and R^{B} are taken together to form

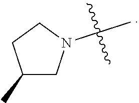 [SMILES: *N1CC[C@H](C)C1]

In some embodiments of a compound of Formula (I), Ring B and R^{B} are taken together to form

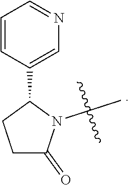 [SMILES: *N1C(=O)CC[C@@H]1c1cccnc1]

In some embodiments of a compound of Formula (I), Ring B and R^{B} are taken together to form

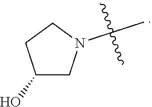 [SMILES: *N1CC[C@@H](O)C1]

In some embodiments of a compound of Formula (I), Ring B and R^{B} are taken together to form

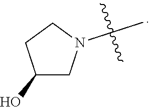 [SMILES: *N1CC[C@H](O)C1]

In some embodiments of a compound of Formula (I), Ring B and R^{B} are taken together to form

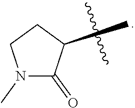

In some embodiments of a compound of Formula (I), Ring B and R^{B} are taken together to form

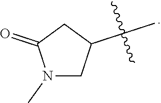 [SMILES: *C1CC(=O)N(C)C1]

In some embodiments of a compound of Formula (I), Ring B and R^{B} are taken together to form

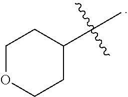 [SMILES: *C1CCOCC1]

In some embodiments of a compound of Formula (I), Ring B and R^{B} are taken together to form

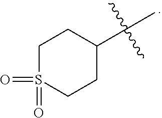 [SMILES: *C1CCS(=O)(=O)CC1]

In some embodiments of a compound of Formula (I), Ring B and R^{B} are taken together to form

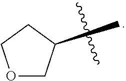

In some embodiments of a compound of Formula (I), Ring B and R^{B} are taken together to form

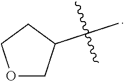 [SMILES: *C1CCOC1]

In some embodiments of a compound of Formula (I), Ring B and R^{B} are taken together to form

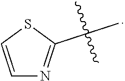 [SMILES: *c1nccs1]

In some embodiments of a compound of Formula (I), Ring B and R^{B} are taken together to form

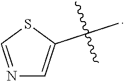 [SMILES: *c1cncs1]

In some embodiments of a compound of Formula (I), Ring B and R^{B} are taken together to form

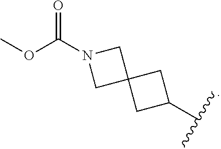 [SMILES: *C1CC2(C1)CN(C(=O)OC)C2]

In some embodiments of a compound of Formula (I), Ring B and R^{B} are taken together to form

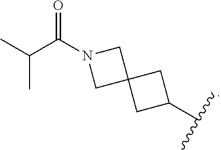 [SMILES: *C1CC2(C1)CN(C(=O)C(C)C)C2]

In some embodiments of a compound of Formula (I), Ring B and R^{B} are taken together to form

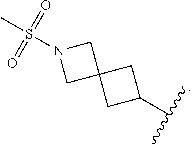 [SMILES: *C1CC2(C1)CN(S(C)(=O)=O)C2]

In some embodiments of a compound of Formula (I), Ring B and R^{B} are taken together to form

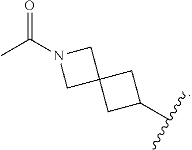 [SMILES: *C1CC2(C1)CN(C(C)=O)C2]

In some embodiments of a compound of Formula (I), Ring B and R^{B} are taken together to form

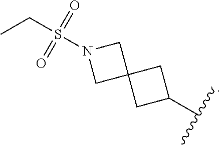 [SMILES: *C1CC2(C1)CN(S(=O)(=O)CC)C2]

In some embodiments of a compound of Formula (I), Ring B and R^{B} are taken together to form

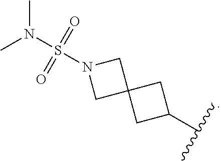 [SMILES: *C1CC2(C1)CN(S(=O)(=O)N(C)C)C2]

In some embodiments of a compound of Formula (I), Ring B and R^{B} are taken together to form

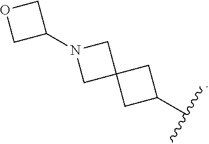 [SMILES: *C1CC2(C1)CN(C1COC1)C2]

In some embodiments of a compound of Formula (I), Ring B and R^{B} are taken together to form

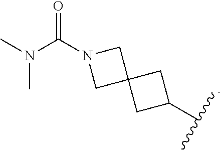 [SMILES: *C1CC2(C1)CN(C(=O)N(C)C)C2]

In some embodiments of a compound of Formula (I), Ring B and R^{B} are taken together to form

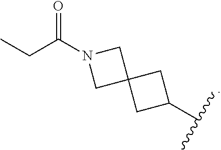 [SMILES: *C1CC2(C1)CN(C(=O)CC)C2]

In some embodiments of a compound of Formula (I), Ring B and R^{B} are taken together to form

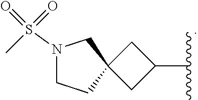

In some embodiments of a compound of Formula (I), Ring B and R^{B} are taken together to form

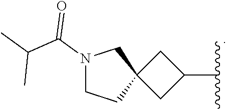

In some embodiments of a compound of Formula (I), Ring B and R^{B} are taken together to form

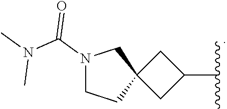

In some embodiments of a compound of Formula (I), Ring B and R^{B} are taken together to form

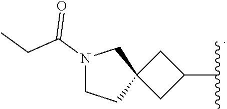

In some embodiments of a compound of Formula (I), Ring B and R^{B} are taken together to form

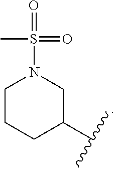 [SMILES: *C1CCCN(S(C)(=O)=O)C1]

In some embodiments of a compound of Formula (I), Ring B and R^{B} are taken together to form

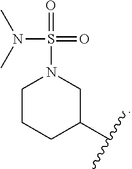 [SMILES: *C1CCCN(S(=O)(=O)N(C)C)C1]

In some embodiments of a compound of Formula (I), Ring B and R^{B} are taken together to form

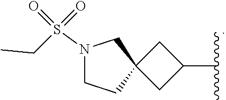

In some embodiments of a compound of Formula (I), Ring B and R^{B} are taken together to form

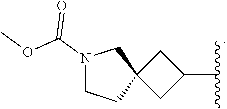

In some embodiments of a compound of Formula (I), Ring B and R^{B} are taken together to form

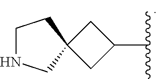

In some embodiments of a compound of Formula (I), Ring B and R^{B} are taken together to form

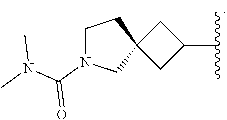

In some embodiments of a compound of Formula (I), Ring B and R^{B} are taken together to form

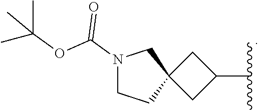

In some embodiments of a compound of Formula (I), Ring B and R^{B} are taken together to form

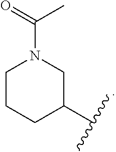 [SMILES: *C1CCCN(C(C)=O)C1]

In some embodiments of a compound of Formula (I), Ring B and R^{B} are taken together to form

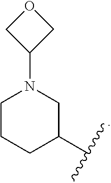 [SMILES: *C1CCCN(C2COC2)C1]

In some embodiments of a compound of Formula (I), Ring B and R^{B} are taken together to form

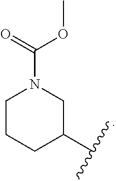 [SMILES: *C1CCCN(C(=O)OC)C1]

In some embodiments of a compound of Formula (I), Ring B and R^{B} are taken together to form

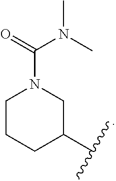 [SMILES: *C1CCCN(C(=O)N(C)C)C1]

In some embodiments of a compound of Formula (I), Ring B and R^{B} are taken together to form

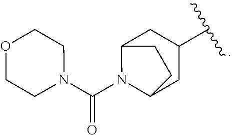 [SMILES: *C1CC2CCC(C1)N2C(=O)N1CCOCC1]

In some embodiments of a compound of Formula (I), Ring B and R^{B} are taken together to form

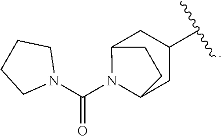 [SMILES: *C1CC2CCC(C1)N2C(=O)N1CCCC1]

In some embodiments of a compound of Formula (I), Ring B and R^{B} are taken together to form

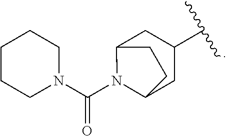 [SMILES: *C1CC2CCC(C1)N2C(=O)N1CCCCC1]

In some embodiments of a compound of Formula (I), Ring B and R^{B} are taken together to form

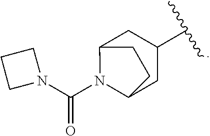 [SMILES: *C1CC2CCC(C1)N2C(=O)N1CCC1]

In some embodiments of a compound of Formula (I), Ring B and R^{B} are taken together to form

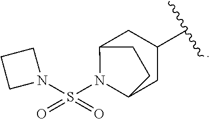 [SMILES: *C1CC2CCC(C1)N2S(=O)(=O)N1CCC1]

In some embodiments of a compound of Formula (I), Ring B and R^{B} are taken together to form

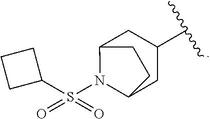 [SMILES: *C1CC2CCC(C1)N2S(=O)(=O)C1CCC1]

In some embodiments of a compound of Formula (I), Ring B and R^{B} are taken together to form

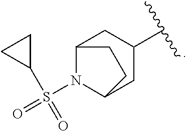 [SMILES: *C1CC2CCC(C1)N2S(=O)(=O)C1CC1]

In some embodiments of a compound of Formula (I), Ring B and R^{B} are taken together to form

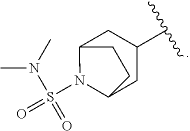 [SMILES: *C1CC2CCC(C1)N2S(=O)(=O)N(C)C]

In some embodiments of a compound of Formula (I), Ring B and R^{B} are taken together to form

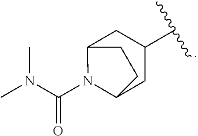 [SMILES: *C1CC2CCC(C1)N2C(=O)N(C)C]

In some embodiments of a compound of Formula (I), Ring B and R^{B} are taken together to form

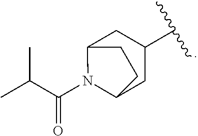 [SMILES: *C1CC2CCC(C1)N2C(=O)C(C)C]

In some embodiments of a compound of Formula (I), Ring B and R^{B} are taken together to form

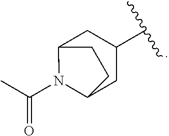 [SMILES: *C1CC2CCC(C1)N2C(C)=O]

In some embodiments of a compound of Formula (I), Ring B and R^{B} are taken together to form

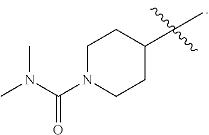 [SMILES: *C1CCN(C(=O)N(C)C)CC1]

In some embodiments of a compound of Formula (I), Ring B and R^{B} are taken together to form

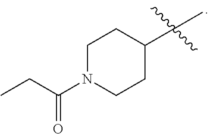 [SMILES: *C1CCN(C(=O)CC)CC1]

In some embodiments of a compound of Formula (I), Ring B and R^{B} are taken together to form

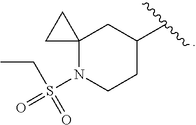 [SMILES: *C1CCN(S(=O)(=O)CC)C2(CC2)C1]

In some embodiments of a compound of Formula (I), Ring B and R^{B} are taken together to form

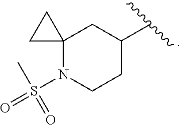 [SMILES: *C1CCN(S(C)(=O)=O)C2(CC2)C1]

In some embodiments of a compound of Formula (I), Ring B and R^{B} are taken together to form

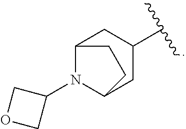 [SMILES: *C1CC2CCC(C1)N2C1COC1]

In some embodiments of a compound of Formula (I), Ring B and R^{B} are taken together to form

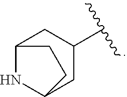 [SMILES: *C1CC2CCC(C1)N2]

In some embodiments of a compound of Formula (I), Ring B and R^{B} are taken together to form

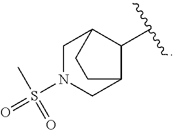 [SMILES: *C1C2CCC1CN(S(C)(=O)=O)C2]

In some embodiments of a compound of Formula (I), Ring B and R^{B} are taken together to form

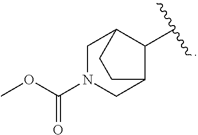 [SMILES: *C1C2CCC1CN(C(=O)OC)C2]

In some embodiments of a compound of Formula (I), Ring B and R^{B} are taken together to form

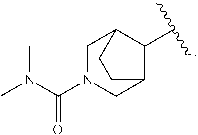 [SMILES: *C1C2CCC1CN(C(=O)N(C)C)C2]

In some embodiments of a compound of Formula (I), Ring B and R^{B} are taken together to form

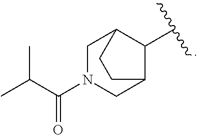 [SMILES: *C1C2CCC1CN(C(=O)C(C)C)C2]

In some embodiments of a compound of Formula (I), Ring B and R^{B} are taken together to form

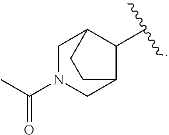 [SMILES: *C1C2CCC1CN(C(C)=O)C2]

In some embodiments of a compound of Formula (I), Ring B and R^{B} are taken together to form

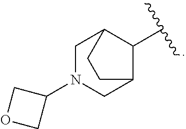 [SMILES: *C1C2CCC1CN(C1COC1)C2]

In some embodiments of a compound of Formula (I), Ring B and R^{B} are taken together to form

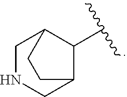 [SMILES: *C1C2CCC1CNC2]

In some embodiments of a compound of Formula (I), Ring B and R^{B} are taken together to form

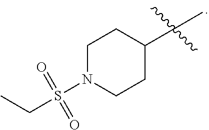 [SMILES: *C1CCN(S(=O)(=O)CC)CC1]

In some embodiments of a compound of Formula (I), Ring B and R^{B} are taken together to form

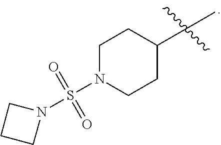 [SMILES: *C1CCN(S(=O)(=O)N2CCC2)CC1]

In some embodiments of a compound of Formula (I), Ring B and R^{B} are taken together to form

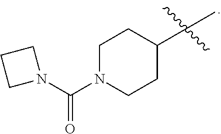 [SMILES: *C1CCN(C(=O)N2CCC2)CC1]

In some embodiments of a compound of Formula (I), Ring B and R^{B} are taken together to form

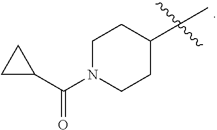 [SMILES: *C1CCN(C(=O)C2CC2)CC1]

In some embodiments of a compound of Formula (I), Ring B and R^{B} are taken together to form

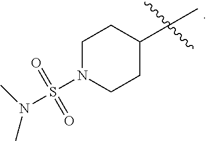 [SMILES: *C1CCN(S(=O)(=O)N(C)C)CC1]

In some embodiments of a compound of Formula (I), Ring B and R^{B} are taken together to form

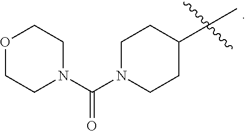 [SMILES: *C1CCN(C(=O)N2CCOCC2)CC1]

In some embodiments of a compound of Formula (I), Ring B and R^{B} are taken together to form

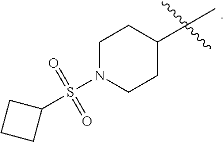 [SMILES: *C1CCN(S(=O)(=O)C2CCC2)CC1]

In some embodiments of a compound of Formula (I), Ring B and R^{B} are taken together to form

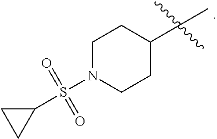 [SMILES: *C1CCN(S(=O)(=O)C2CC2)CC1]

In some embodiments of a compound of Formula (I), Ring B and R^{B} are taken together to form

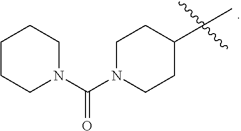 [SMILES: *C1CCN(C(=O)N2CCCCC2)CC1]

In some embodiments of a compound of Formula (I), Ring B and R^{B} are taken together to form

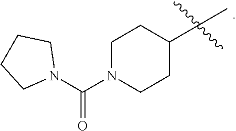 [SMILES: *C1CCN(C(=O)N2CCCC2)CC1]

In some embodiments of a compound of Formula (I), Ring B and R^{B} are taken together to form

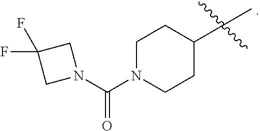 [SMILES: *C1CCN(C(=O)N2CC(F)(F)C2)CC1]

In some embodiments of a compound of Formula (I), Ring B and R^{B} are taken together to form

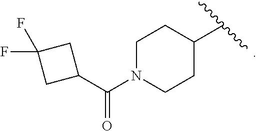 [SMILES: *C1CCN(C(=O)C2CC(F)(F)C2)CC1]

In some embodiments of a compound of Formula (I), Ring B and R^{B} are taken together to form

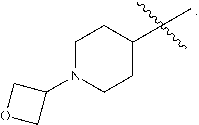 [SMILES: *C1CCN(C2COC2)CC1]

In some embodiments of a compound of Formula (I), Ring B and R^{B} are taken together to form

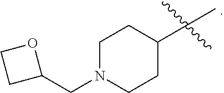 [SMILES: *C1CCN(CC2CCO2)CC1]

In some embodiments of a compound of Formula (I), Ring B and R^{B} are taken together to form

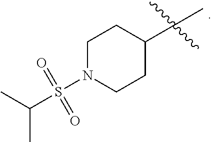 [SMILES: *C1CCN(S(=O)(=O)C(C)C)CC1]

In some embodiments of a compound of Formula (I), Ring B and R^{B} are taken together to form

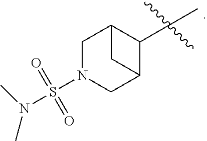 [SMILES: *C1C2CC1CN(S(=O)(=O)N(C)C)C2]

In some embodiments of a compound of Formula (I), Ring B and R^{B} are taken together to form

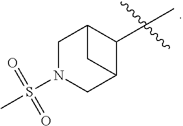 [SMILES: *C1C2CC1CN(S(C)(=O)=O)C2]

In some embodiments of a compound of Formula (I), Ring B and R^{B} are taken together to form

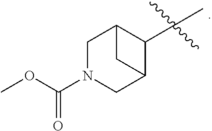 [SMILES: *C1C2CC1CN(C(=O)OC)C2]

In some embodiments of a compound of Formula (I), Ring B and R^{B} are taken together to form

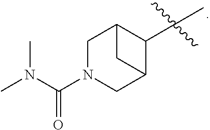 [SMILES: *C1C2CC1CN(C(=O)N(C)C)C2]

In some embodiments of a compound of Formula (I), Ring B and R^{B} are taken together to form

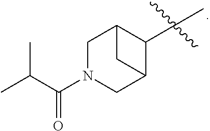 [SMILES: *C1C2CC1CN(C(=O)C(C)C)C2]

In some embodiments of a compound of Formula (I), Ring B and R^{B} are taken together to form

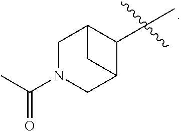 [SMILES: *C1C2CC1CN(C(C)=O)C2]

In some embodiments of a compound of Formula (I), Ring B and R^{B} are taken together to form

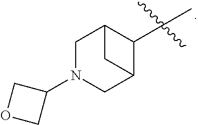 [SMILES: *C1C2CC1CN(C1COC1)C2]

In some embodiments of a compound of Formula (I), Ring B and R^{B} are taken together to form

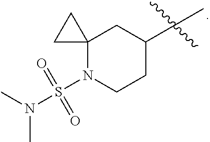 [SMILES: *C1CCN(S(=O)(=O)N(C)C)C2(CC2)C1]

In some embodiments of compound of Formula (I), Ring B and R^{B} are taken together to form

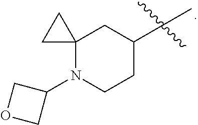 [SMILES: *C1CCN(C2COC2)C2(CC2)C1]

In some embodiments of a compound of Formula (I), Ring B and R^{B} are taken together to form

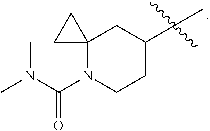 [SMILES: *C1CCN(C(=O)N(C)C)C2(CC2)C1]

In some embodiments of a compound of Formula (I), Ring B and R^{B} are taken together to form

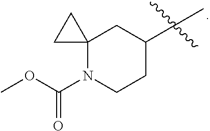 [SMILES: *C1CCN(C(=O)OC)C2(CC2)C1]

In some embodiments of a compound of Formula (I), Ring B and R^{B} are taken together to form

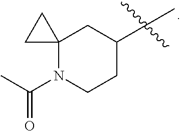 [SMILES: *C1CCN(C(C)=O)C2(CC2)C1]

In some embodiments of a compound of Formula (I), Ring B and R^{B} are taken together to form

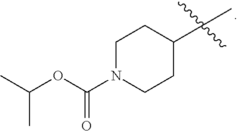 [SMILES: *C1CCN(C(=O)OC(C)C)CC1]

In some embodiments of a compound of Formula (I), Ring B and R^{B} are taken together to form

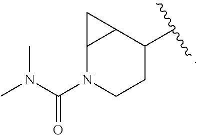 [SMILES: *C1CCN(C(=O)N(C)C)C2CC12]

In some embodiments of a compound of Formula (I), Ring B and R^{B} are taken together to form

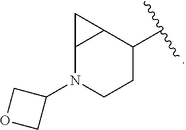 [SMILES: *C1CCN(C2COC2)C2CC12]

In some embodiments of a compound of Formula (I), Ring B and R^{B} are taken together to form

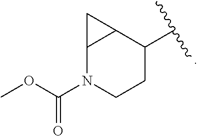 [SMILES: *C1CCN(C(=O)OC)C2CC12]

In some embodiments of a compound of Formula (I), Ring B and R^{B} are taken together to form

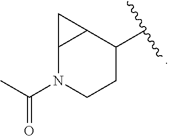 [SMILES: *C1CCN(C(C)=O)C2CC12]

In some embodiments of a compound of Formula (I), L is selected from the group consisting of a bond, C₁-C₆ alkylene, #—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$, #—C(O)—N(R^{L})—(C₁-C₆ alkylene)-$,
#—(C₁-C₆ alkylene)-C(O)—N(R^{L})—$, #—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$,
#—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule; wherein each C₁-C₆ alkylene of L is optionally substituted with 1 to 3 substituents independently selected from the group consisting of halogen, —OH, and C₁-C₆ alkyl; and wherein each R^{L} is independently hydrogen or C₁-C₆ alkyl.

In some embodiments, L is selected from the group consisting of a bond, C₁-C₃ alkylene, #—O—(C₁-C₃ alkylene)-$, #—C(O)—(C₁-C₃ alkylene)-$,
#—(C₁-C₃ alkylene)-C(O)—$, #—N(R^{L})—(C₁-C₃ alkylene)-$, #—(C₁-C₃ alkylene)-N(R^{L})—$,
#—(C₁-C₃ alkylene)-N(R^{L})—(C₁-C₃ alkylene)-$, #—C(O)—N(R^{L})—(C₁-C₃ alkylene)-$,
#—(C₁-C₃ alkylene)-C(O)—N(R^{L})—$, #—N(R^{L})—C(O)—CH₂-$, #—(C₁-C₃ alkylene)-N(R^{L})—C(O)—$,
#—(C₁-C₃ alkylene)-C(O)—N(R^{L})—(C₁-C₃ alkylene)-$,
#—(C₁-C₃ alkylene)-N(R^{L})—C(O)—(C₁-C₃ alkylene)-$, #—(C₁-C₃ alkylene)-N(R^{L})—S(O)₂-$,
#—N(R^{L})—S(O)₂—(C₁-C₃ alkylene)-$, #—(C₁-C₃ alkylene)-S(O)₂—N(R^{L})—$,
#—S(O)₂—N(R^{L})—(C₁-C₃ alkylene)-$, #—S(O)₂—N(R^{L})—$, and #—N(C₁-C₃ alkyl)-S(O)₂-$, wherein #represents the attachment point to ring B, and $ represents the attachment point to the remainder of the molecule; wherein each C₁-C₃ alkylene of L is optionally substituted with 1 to 3 substituents independently selected from the group consisting of halogen, —OH, and C₁-C₃ alkyl; and wherein each R^{L} is independently hydrogen or C₁-C₃ alkyl.

In some embodiments, L is selected from the group consisting of a bond, C₁-C₆ alkylene, #—C(O)—N(R^{L})—(C₁-C₆ alkylene)-$, #—C(O)—(C₁-C₆ alkylene)-$, and #—(C₁-C₆ alkylene)-C(O)—N(R^{L})—(C₁-C₆ alkylene)-$, wherein #represents the attachment point to ring B, and $ represents the attachment point to the remainder of the molecule; wherein each C₁-C₆ alkylene of L is optionally substituted with 1 to 3 substituents independently selected from the group consisting of halogen, —OH, and C₁-C₆ alkyl; and wherein each R^{L} is independently hydrogen or C₁-C₆ alkyl.

In some embodiments, L is a bond, C₁-C₃ alkylene, #—C(O)—N(R^{L})—(C₁-C₃ alkylene)-$, #—C(O)—(C₁-C₃ alkylene)-$, or #—(C₁-C₃ alkylene)-C(O)—N(R^{L})—(C₁-C₃ alkylene)-$, wherein #represents the attachment point to ring B, and $ represents the attachment point to the remainder of the molecule; wherein each C₁-C₃ alkylene of L is optionally substituted with 1 to 3 substituents independently selected from the group consisting of halogen, —OH, and C₁-C₃ alkyl; and wherein each R^{L} is independently hydrogen or C₁-C₃ alkyl.

In some embodiments, each C₁-C₆ alkylene of L is substituted with 1 to 3 substituents independently selected from the group consisting of halogen, —OH, and C₁-C₆ alkyl. In some embodiments, each C₁-C₆ alkylene of L is substituted with 1 to 3 substituents independently selected from the group consisting of halogen and C₁-C₆ alkyl. In some embodiments, each C₁-C₆alkylene of L is substituted with 1 to 3 substituents independently selected from the group consisting of fluoro and methyl. In some embodiments, each C₁-C₆ alkylene of L is unsubstituted.

In some embodiments, L is a bond, C₁-C₃ alkylene, or #—C(O)—N(R^{L})—(C₁-C₃ alkylene)-$, wherein #represents the attachment point to ring B, and $ represents the attachment point to the remainder of the molecule; wherein each C₁-C₆ alkylene of L is optionally substituted with 1 to 3 substituents independently selected from the group consisting of halogen, —OH, and C₁-C₆ alkyl; and wherein each R^{L} is independently hydrogen or C₁-C₆ alkyl. In some embodiments, L is a bond, —CH₂—, —CH₂CH₂—, or

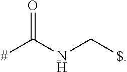

In some embodiments, L is selected from the group consisting of a bond, #—(CO)CF₂-$, #C(O)CH(CH₃)-$, #C(O)CH₂-$, #—C(O)—N(CH₃)CH(CH₃)-$, #—C(O)—N(CH₃)CH₂-$, #—C(O)N(H)CH(CH₃)-$, #—C(O)N(H)CH₂-$, #—CH₂C(O)N(H)CH(CH₃)-$,
#—CH₂C(O)N(H)CH₂-$, #—CH₂N(CH₃)SO₂-$, #—CH₂N(H)C(O)CH₂-$, #—CH₂SO₂N(H)-$,
#—N(CH₂CH₃)SO₂-$, #—N(CH₃)SO₂-$, #—N(CH₃)C(O)CH₂-$, #—N(H)C(O)CH₂-$,
#—N(H)CH₂-$, #—OCH₂-$, #—S(O)₂N(H)CH₂-$, #—C(CH₃)(OH)-$, ethylene, methylene, and
#—S(O)₂-$. In some embodiments, L is a bond. In some embodiments, L is #—(CO)CF₂-$. In some embodiments, L is #C(O)CH(CH₃)-$. In some embodiments, L is #C(O)CH₂-$. In some embodiments, L is #—C(O)—N(CH₃)CH(CH₃)-$. In some embodiments, L is #—C(O)—N(CH₃)CH₂-$. In some embodiments, L is #—C(O)N(H)CH(CH₃)-$. In some embodiments, L is #—C(O)N(H)CH₂-$. In some embodiments, L is #—CH₂C(O)N(H)CH(CH₃)-$. In some embodiments, L is #—CH₂C(O)N(H)CH₂-$. In some embodiments, L is #—CH₂N(CH₃)SO₂-$. In some embodiments, L is #—CH₂N(H)C(O)CH₂-$. In some embodiments, L is #—CH₂SO₂N(H)-$. In some embodiments, L is #—N(CH₂CH₃)SO₂-$. In some embodiments, L is #—N(CH₃)SO₂-$. In some embodiments, L is #—N(CH₃)C(O)CH₂-$. In some embodiments, L is #—N(H)C(O)CH₂-$. In some embodiments, L is #—N(H)CH₂-$. In some embodiments, L is #—OCH₂-$. In some embodiments, L is #—S(O)₂N(H)CH₂-$. In some embodiments, L is #—C(CH₃)(OH)-$. In some embodiments, L is ethylene. In some embodiments, L is methylene. In some embodiments, L is #—S(O)₂-$.

In some embodiments of a compound of Formula (I), m is 0, 1, 2, 3, or 4. In some embodiments, m is 0. In some embodiments, m is 1. In some embodiments, m is 2. In some embodiments, m is 3. In some embodiments, m is 4.

In some embodiments of a compound of Formula (I), n is 0, 1, 2, 3, 4, or 5. In some embodiments, n is 0. In some embodiments, n is 1. In some embodiments, n is 2. In some embodiments, n is 3. In some embodiments, n is 4. In some embodiments, n is 5.

In some embodiments of a compound of Formula (I), p is 0, 1, 2, 3, or 4. In some embodiments, p is 0. In some embodiments, p is 1. In some embodiments, p is 2. In some embodiments, p is 3. In some embodiments, p is 4.

In some embodiments of a compound of Formula (I), p is 0, m is 0, and n is 0, 1, or 2.

In some embodiments of a compound of Formula (I), R^{C} is halogen, cyano, C₁-C₆ alkyl, or 3- to 8-membered cycloalkyl. In some embodiments, R^{C} is halogen. In some embodiments, R^{C} is fluoro, chloro, or bromo. In some embodiments, R^{C} is fluoro. In some embodiments, R^{C} is chloro. In some embodiments, R^{C} is bromo. In some embodiments, R^{C} is cyano. In some embodiments, R^{C} is C₁-C₆ alkyl. In some embodiments, R^{C} is methyl. In some embodiments, R^{C} is ethyl. In some embodiments, R^{C} is propyl. In some embodiments, R^{C} is cyclopropyl. In some embodiments, R^{C} is cyclobutyl. In some embodiments, R^{C} is cyclopentyl. In some embodiments, R^{C} is cyclohexyl. In some embodiments, R^{C} is cyclopentyl. In some embodiments, R^{C} is cyclooctyl.

In some embodiments of a compound of Formula (I):
a) when L is a bond, ring B is selected from the group consisting of

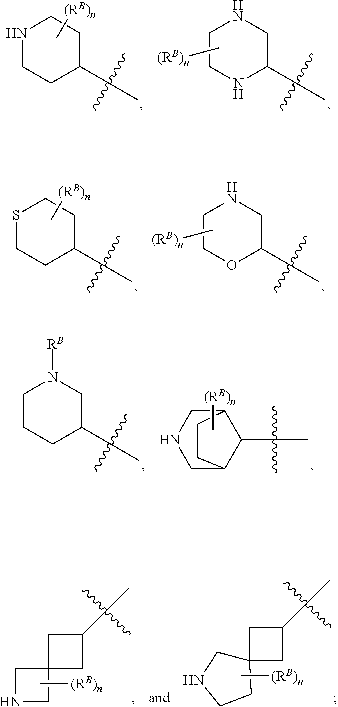

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

In some embodiments of a compound of Formula (I), each R^{B} is independently selected from the group consisting of: halogen; —OH; oxo; cyano; —O(C₁-C₆ alkyl) optionally substituted with phenyl or 1 to 3 independently selected halogens;
—C(O)(C₁-C₃ alkyl); —C(O)O(C₁-C₆ alkyl); phenyl; 5- to 6-membered heteroaryl; 4- to 8-membered heterocycloalkyl; 3- to 8-membered cycloalkyl; —C(O)(3- to 8-membered cycloalkyl);
—C(O)(4-8-membered heterocycloalkyl); —S(O)₂(C₁-C₆ alkyl); —S(O)₂(3- to 8-membered cycloalkyl); —C(O)NR^{B1}R^{B2}; —S(O)₂NR^{B1}R^{B2}; —NR^{C1}S(O)₂NR^{B1}R^{B2};
—(C═N—R^{C1})—NR^{B1}R^{B2}; —NR^{C1}—(C═N—R^{C})—NR^{B1}R^{B2}; —NR^{C1}—(C═N—CN)—NR^{B1}R^{B2}; and C₁-C₆ alkyl optionally substituted with 1 to 5 substituents independently selected from halogen, —OH, and —O(C₁-C₆ alkyl); wherein R^{B1} and R^{B2} are each independently hydrogen or C₁-C₆ alkyl.

In some embodiments of a compound of Formula (I):
a) when L is a bond, ring B is selected from the group consisting of

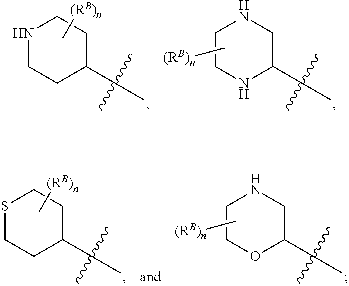

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

In some embodiments of a compound of Formula (I), each R^{B} is independently selected from the group consisting of: halogen; —OH; oxo; cyano; —O(C₁-C₆ alkyl) optionally substituted with phenyl or 1 to 3 independently selected halogens;
—C(O)(C₁-C₃ alkyl); —C(O)O(C₁-C₆ alkyl); phenyl; 5- to 6-membered heteroaryl; 4- to 8-membered heterocycloalkyl; 3- to 8-membered cycloalkyl; —C(O)(3- to 8-membered cycloalkyl);
—C(O)(4-8-membered heterocycloalkyl); —S(O)₂(C₁-C₆ alkyl); —S(O)₂(3- to 8-membered cycloalkyl); —C(O)NR^{B1}R^{B2}; —S(O)₂NR^{B1}R^{B2}; —NR^{C1}S(O)₂NR^{B1}R^{B2};
—(C═N—R^{C1})—NR^{B1}R^{B2}; —NR^{C1}—(C═N—R^{C})—NR^{B1}R^{B2}; —NR^{C1}—(C═N—CN)—NR^{B1}R^{B2}; and C₁-C₆ alkyl optionally substituted with 1 to 5 substituents independently selected from halogen, —OH, and —O(C₁-C₆ alkyl); wherein R^{B1} and R^{B2} are each independently hydrogen or C₁-C₆ alkyl.

In some embodiments of a compound of Formula (I), Ring B is 4- to 8-membered heterocycloalkyl and R^{B} is 5- to 6-membered heteroaryl. In some embodiments, Ring B is 4- to 8-membered heterocycloalkyl and R^{B} is —S(O)₂(C₁-C₆ alkyl). In some embodiments, Ring B is 4- to 8-membered heterocycloalkyl and R^{B} is —S(O)₂(C₁-C₃ alkyl). In some embodiments, Ring B is 4- to 8-membered heterocycloalkyl and R^{B} is —S(O)₂(Me). In some embodiments, Ring B is 4- to 8-membered heterocycloalkyl and R^{B} is-S(O)₂NR^{B1}R^{B2}. In some embodiments, Ring B is 4- to 8-membered heterocycloalkyl and R^{B} is —S(O)₂NMe₂. In some embodiments, Ring B is 4- to 8-membered heterocycloalkyl and R^{B} is —C(O)(C₁-C₆ alkyl). In some embodiments, Ring B is 4- to 8-membered heterocycloalkyl and R^{B} is —C(O)(C₁-C₃ alkyl). In some embodiments, Ring B is 4- to 8-membered heterocycloalkyl and R^{B} is —C(O)Me. In some embodiments, Ring B is 4- to 8-membered heterocycloalkyl and R^{B} is —C(O)NR^{B1}R^{B2}. In some embodiments, Ring B is 4- to 8-membered heterocycloalkyl and R^{B} is 4- to 8-membered heterocycloalkyl. In some embodiments, Ring B is 4- to 8-membered heterocycloalkyl and R^{B} is oxetanyl. In some embodiments, Ring B is 4- to 8-membered heterocycloalkyl and L is #—C(O)—N(R^{L})—(C₁-C₆ alkylene)-$. In some embodiments, Ring B is 4- to 8-membered heterocycloalkyl and L is C₁-C₆ alkylene. In some embodiments, Ring B is 4- to 8-membered heterocycloalkyl and L is a bond. In some embodiments, Ring B is 4- to 8-membered heterocycloalkyl and n is 0. In some embodiments, Ring B is 4- to 8-membered heterocycloalkyl, n is 2, one R^{B} is C₁-C₆ alkyl, and the other R^{B} is oxo. In some embodiments, Ring B is 5- to 6-membered heteroaryl and R^{B} is C₁-C₆ alkyl. In some embodiments, Ring B is 5- to 6-membered heteroaryl and R^{B} is methyl. In some embodiments, Ring B is 5- to 6-membered heteroaryl and L is #—C(O)—N(R^{L})—(C₁-C₆ alkylene)-$. In some embodiments, Ring B is 5- to 6-membered heteroaryl and L is C₁-C₆ alkylene. In some embodiments, Ring A is phenyl and R^{D} is chloro. In some embodiments, Ring A is 5- to 6-membered heteroaryl and m is 0. In some embodiments, Ring A is thiazolyl and m is 0. In some embodiments, Ring A is oxazolyl and m is 0. In some embodiments, Ring A is thiazol-5-yl and m is 0. In some embodiments, Ring A is oxazol-5-yl and m is 0.

In some embodiments of a compound of Formula (I), Ring B is 4- to 8-membered heterocycloalkyl and R^{B} is 5- to 6-membered heteroaryl. In some embodiments, Ring B is 4- to 8-membered heterocycloalkyl and R^{B} is —S(O)₂(C₁-C₆ alkyl). In some embodiments, Ring B is 4- to 8-membered heterocycloalkyl and R^{B} is —S(O)₂(C₁-C₃ alkyl). In some embodiments, Ring B is 4- to 8-membered heterocycloalkyl and R^{B} is-S(O)₂NR^{B1}R^{B2}. In some embodiments, Ring B is 4- to 8-membered heterocycloalkyl and R^{B} is —C(O)(C₁-C₆ alkyl). In some embodiments, Ring B is 4- to 8-membered heterocycloalkyl and R^{B} is —C(O)(C₁-C₃ alkyl). In some embodiments, Ring B is 4- to 8-membered heterocycloalkyl and R^{B} is —C(O)NR^{B1}R^{B2}. In some embodiments, Ring B is 4- to 8-membered heterocycloalkyl and R^{B} is 4- to 8-membered heterocycloalkyl. In some embodiments, Ring B is 4- to 8-membered heterocycloalkyl and L is #—C(O)—N(R^{L})—(C₁-C₆ alkylene)-$. In some embodiments, Ring B is 4- to 8-membered heterocycloalkyl and L is C₁-C₆ alkylene. In some embodiments, Ring B is 4- to 8-membered heterocycloalkyl and L is a bond. In some embodiments, Ring B is 4- to 8-membered heterocycloalkyl and n is 0. In some embodiments, Ring B is 4- to 8-membered heterocycloalkyl, n is 2, one R^{B} is C₁-C₆ alkyl, and the other R^{B} is oxo. In some embodiments, Ring B is 5- to 6-membered heteroaryl and R^{B} is C₁-C₆ alkyl. In some embodiments, Ring B is 5- to 6-membered heteroaryl and R^{B} is methyl. In some embodiments, Ring B is 5- to 6-membered heteroaryl and L is #—C(O)—N(R^{L})—(C₁-C₆ alkylene)-$. In some embodiments, Ring B is 5- to 6-membered heteroaryl and L is C₁-C₆ alkylene. In some embodiments, Ring A is phenyl and R^{D} is chloro. In some embodiments, Ring A is 5- to 6-membered heteroaryl and m is 0. In some embodiments, Ring A is thiazolyl and m is 0. In some embodiments, Ring A is oxazolyl and m is 0. In some embodiments, Ring A is thiazol-5-yl and m is 0. In some embodiments, Ring A is oxazol-5-yl and m is 0.

In some embodiments, the compound of Formula (I) is selected from the group consisting of:

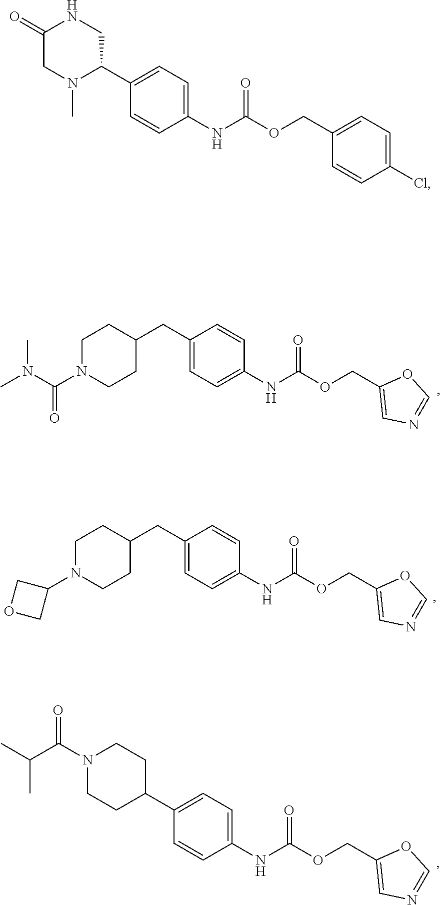 [SMILES: CN1CC(=O)NC[C@@H]1c1ccc(NC(=O)OCc2ccc(Cl)cc2)cc1 ; CN(C)C(=O)N1CCC(Cc2ccc(NC(=O)OCc3cnco3)cc2)CC1 ; O=C(Nc1ccc(CC2CCN(C3COC3)CC2)cc1)OCc1cnco1 ; CC(C)C(=O)N1CCC(c2ccc(NC(=O)OCc3cnco3)cc2)CC1]

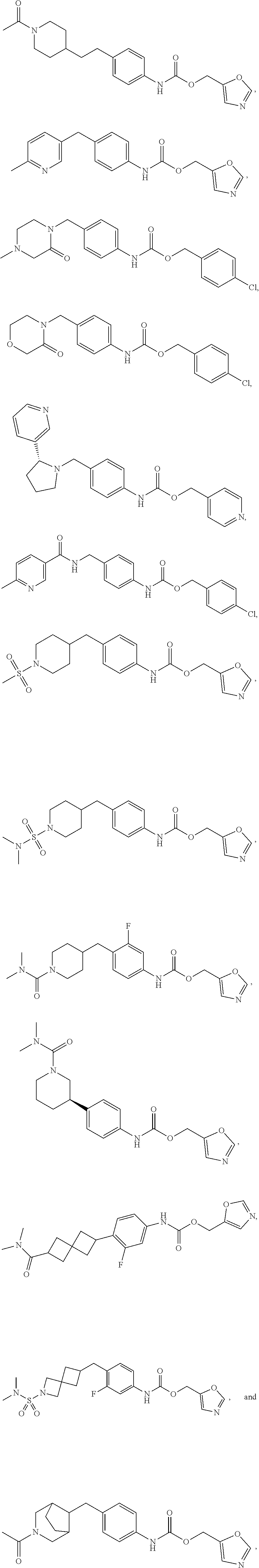

or a pharmaceutically acceptable salt thereof. In some embodiments, the compound is

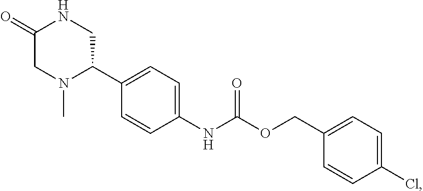 [SMILES: CN1CC(=O)NC[C@@H]1c1ccc(NC(=O)OCc2ccc(Cl)cc2)cc1]

or a pharmaceutically acceptable salt thereof. In some embodiments, the compound is

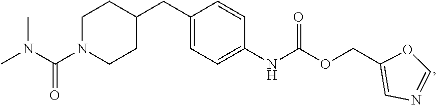 [SMILES: CN(C)C(=O)N1CCC(Cc2ccc(NC(=O)OCc3cnco3)cc2)CC1]

or a pharmaceutically acceptable salt thereof. In some embodiments, the compound is

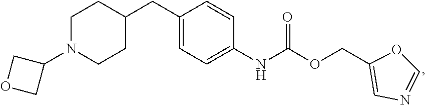 [SMILES: O=C(Nc1ccc(CC2CCN(C3COC3)CC2)cc1)OCc1cnco1]

or a pharmaceutically acceptable salt thereof. In some embodiments, the compound is

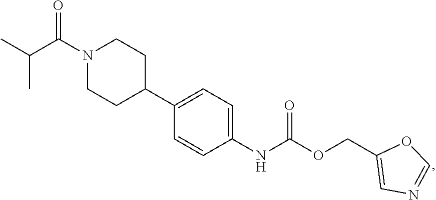 [SMILES: CC(C)C(=O)N1CCC(c2ccc(NC(=O)OCc3cnco3)cc2)CC1]

or a pharmaceutically acceptable salt thereof. In some embodiments, the compound is

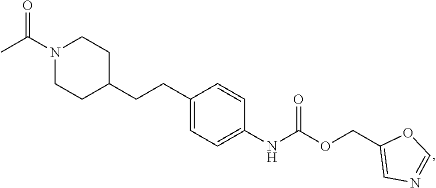 [SMILES: CC(=O)N1CCC(CCc2ccc(NC(=O)OCc3cnco3)cc2)CC1]

or a pharmaceutically acceptable salt thereof. In some embodiments, the compound is

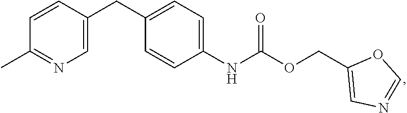 [SMILES: Cc1ccc(Cc2ccc(NC(=O)OCc3cnco3)cc2)cn1]

or a pharmaceutically acceptable salt thereof. In some embodiments, the compound is

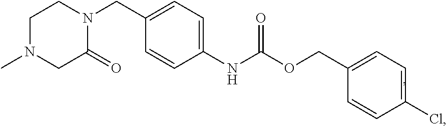 [SMILES: CN1CCN(Cc2ccc(NC(=O)OCc3ccc(Cl)cc3)cc2)C(=O)C1]

or a pharmaceutically acceptable salt thereof. In some embodiments, the compound is

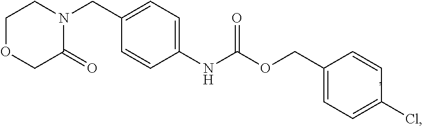 [SMILES: O=C(Nc1ccc(CN2CCOCC2=O)cc1)OCc1ccc(Cl)cc1]

or a pharmaceutically acceptable salt thereof. In some embodiments, the compound is

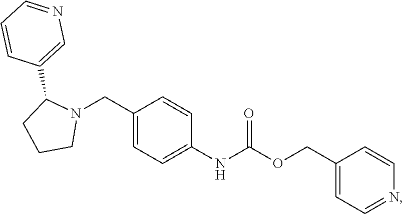 [SMILES: O=C(Nc1ccc(CN2CCC[C@@H]2c2cccnc2)cc1)OCc1ccncc1]

or a pharmaceutically acceptable salt thereof. In some embodiments, the compound is

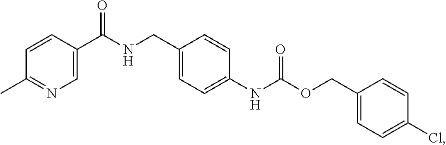 [SMILES: Cc1ccc(C(=O)NCc2ccc(NC(=O)OCc3ccc(Cl)cc3)cc2)cn1]

or a pharmaceutically acceptable salt thereof. In some embodiments, the compound

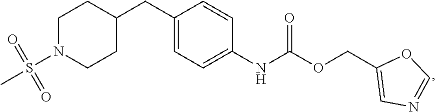 [SMILES: CS(=O)(=O)N1CCC(Cc2ccc(NC(=O)OCc3cnco3)cc2)CC1]

or a pharmaceutically acceptable salt thereof. In some embodiments, the compound is

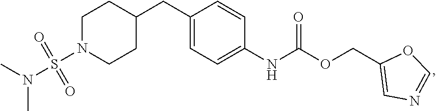 [SMILES: CN(C)S(=O)(=O)N1CCC(Cc2ccc(NC(=O)OCc3cnco3)cc2)CC1]

or a pharmaceutically acceptable salt thereof. In some embodiments, the compound is

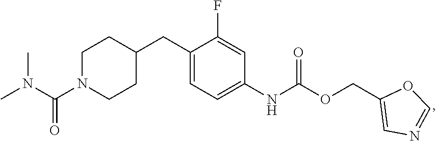 [SMILES: CN(C)C(=O)N1CCC(Cc2ccc(NC(=O)OCc3cnco3)cc2F)CC1]

or a pharmaceutically acceptable salt thereof. In some embodiments, the compound is

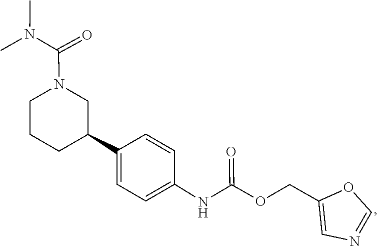 [SMILES: CN(C)C(=O)N1CCC[C@H](c2ccc(NC(=O)OCc3cnco3)cc2)C1]

or a pharmaceutically acceptable salt thereof. In some embodiments, the compound is

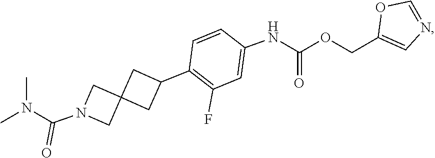 [SMILES: CN(C)C(=O)N1CC2(CC(c3ccc(NC(=O)OCc4cnco4)cc3F)C2)C1]

or a pharmaceutically acceptable salt thereof. In some embodiments, the compound is

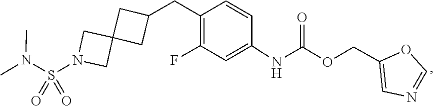 [SMILES: CN(C)S(=O)(=O)N1CC2(CC(Cc3ccc(NC(=O)OCc4cnco4)cc3F)C2)C1]

or a pharmaceutically acceptable salt thereof. In some embodiments, the compound is

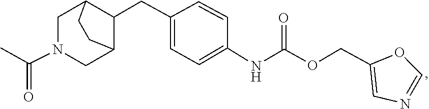 [SMILES: CC(=O)N1CC2CCC(C1)C2Cc1ccc(NC(=O)OCc2cnco2)cc1]

or a pharmaceutically acceptable salt thereof.

In some embodiments, provided herein are compounds, and salts thereof or pharmaceutically acceptable salts thereof, described in Table 1.

TABLE 1

| No. | Structure | Name |
|---|---|---|
| 1 | 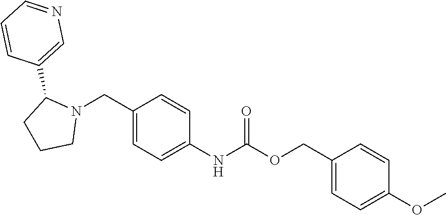 [SMILES: COc1ccc(COC(=O)Nc2ccc(CN3CCC[C@@H]3c3cccnc3)cc2)cc1] | 4-methoxybenzyl (R)-(4- ((2-(pyridin-3- yl)pyrrolidin-1- yl)methyl)phenyl)carbamate |
| 2 | 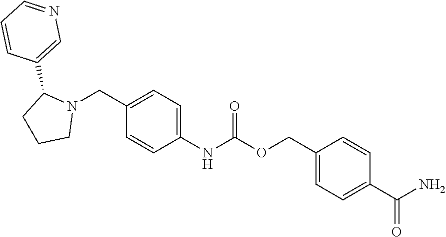 [SMILES: NC(=O)c1ccc(COC(=O)Nc2ccc(CN3CCC[C@@H]3c3cccnc3)cc2)cc1] | 4-carbamoylbenzyl (R)- (4-((2-(pyridin-3- yl)pyrrolidin-1- yl)methyl)phenyl)carbamate |
| 3 | 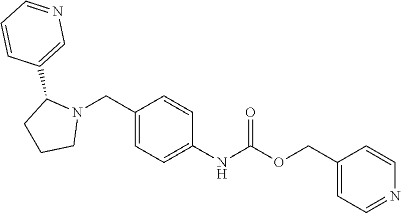 [SMILES: O=C(Nc1ccc(CN2CCC[C@@H]2c2cccnc2)cc1)OCc1ccncc1] | pyridin-4-ylmethyl (R)- (4-((2-(pyridin-3- yl)pyrrolidin-1- yl)methyl)phenyl)carbamate |
| 4 | 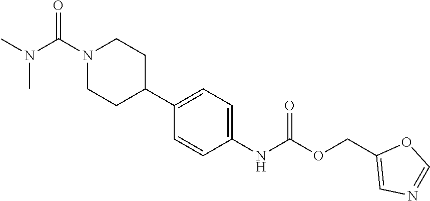 [SMILES: CN(C)C(=O)N1CCC(c2ccc(NC(=O)OCc3cnco3)cc2)CC1] | oxazol-5-ylmethyl (4-(1- (dimethylcarbamoyl)piperidin-4- yl)phenyl)carbamate |
| 5 | 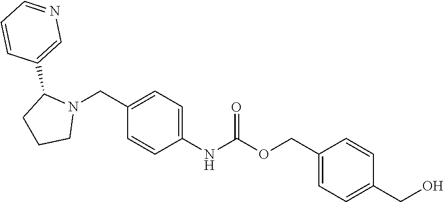 [SMILES: O=C(Nc1ccc(CN2CCC[C@@H]2c2cccnc2)cc1)OCc1ccc(CO)cc1] | 4-(hydroxymethyl)benzyl (R)-(4-((2-(pyridin-3- yl)pyrrolidin-1- yl)methyl)phenyl)carbamate |
| 6 | 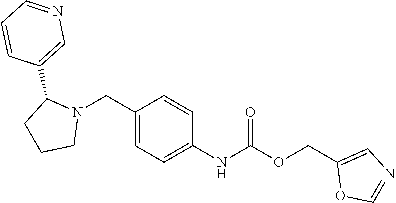 [SMILES: O=C(Nc1ccc(CN2CCC[C@@H]2c2cccnc2)cc1)OCc1cnco1] | oxazol-5-ylmethyl (R)- (4-((2-(pyridin-3- yl)pyrrolidin-1- yl)methyl)phenyl)carbamate |
| 7 | 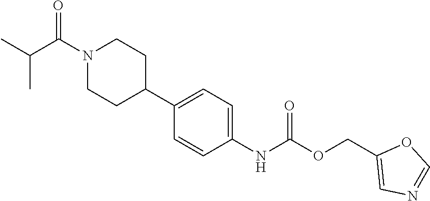 [SMILES: CC(C)C(=O)N1CCC(c2ccc(NC(=O)OCc3cnco3)cc2)CC1] | oxazol-5-ylmethyl (4-(1- isobutyrylpiperidin-4- yl)phenyl)carbamate |
| 8 | 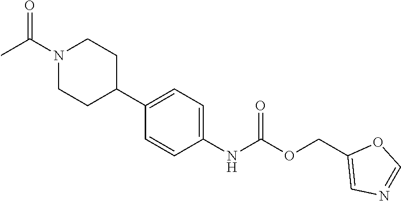 [SMILES: CC(=O)N1CCC(c2ccc(NC(=O)OCc3cnco3)cc2)CC1] | oxazol-5-ylmethyl (4-(1- acetylpiperidin-4- yl)phenyl)carbamate |
| 9 | 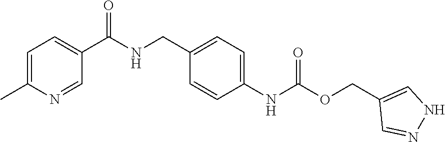 [SMILES: Cc1ccc(C(=O)NCc2ccc(NC(=O)OCc3cn[nH]c3)cc2)cn1] | (1H-pyrazol-4-yl)methyl (4-((6-methyl- nicotinamido)meth- yl)phenyl)carbamate |
| 10 | 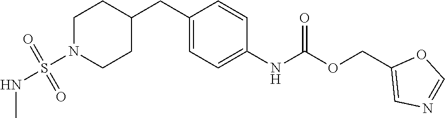 [SMILES: CNS(=O)(=O)N1CCC(Cc2ccc(NC(=O)OCc3cnco3)cc2)CC1] | oxazol-5-ylmethyl (4-((1-(N- methylsulfamoyl)piperidin-4- yl)methyl)phenyl)carbamate |
| 11 | 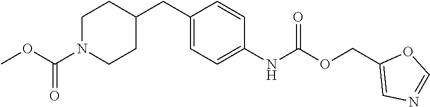 [SMILES: COC(=O)N1CCC(Cc2ccc(NC(=O)OCc3cnco3)cc2)CC1] | methyl 4-(4-(((oxazol-5- ylmethoxy)carbonyl)ami- no)benzyl)piperidine-1- carboxylate |
| 12 | 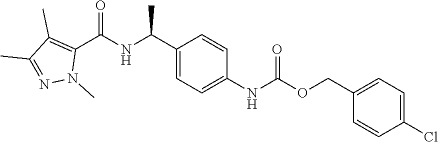 [SMILES: Cc1nn(C)c(C(=O)N[C@@H](C)c2ccc(NC(=O)OCc3ccc(Cl)cc3)cc2)c1C] | 4-chlorobenzyl (S)-(4-(1- (1,3,4-trimethyl-1H- pyrazole-5-carboxamido)eth- yl)phenyl)carbamate |
| 13 | 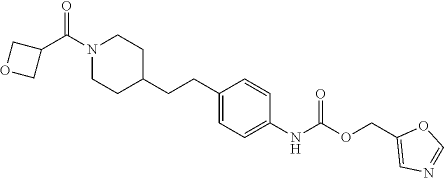 [SMILES: O=C(Nc1ccc(CCC2CCN(C(=O)C3COC3)CC2)cc1)OCc1cnco1] | oxazol-5-ylmethyl (4-(2- (1-(oxetane-3- carbonyl)piperidin-4- yl)ethyl)phenyl)carbamate |
| 14 | 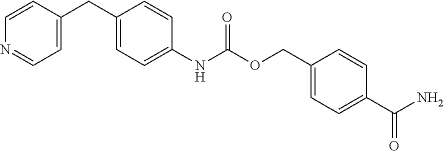 [SMILES: NC(=O)c1ccc(COC(=O)Nc2ccc(Cc3ccncc3)cc2)cc1] | 4-carbamoylbenzyl (4- (pyridin-4- ylmethyl)phenyl)carbamate |
| 15 | 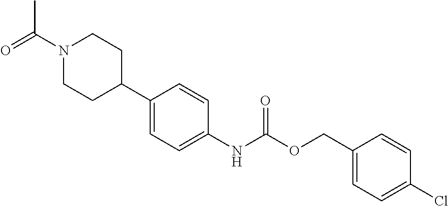 [SMILES: CC(=O)N1CCC(c2ccc(NC(=O)OCc3ccc(Cl)cc3)cc2)CC1] | 4-chlorobenzyl (4-(1- acetylpiperidin-4- yl)phenyl)carbamate |
| 16 | 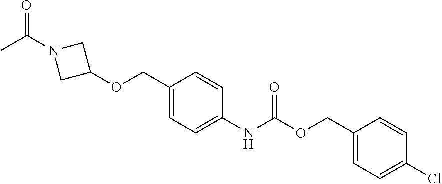 [SMILES: CC(=O)N1CC(OCc2ccc(NC(=O)OCc3ccc(Cl)cc3)cc2)C1] | 4-chlorobenzyl (4-(((1- acetylazetidin-3- yl)oxy)methyl)phenyl)carbamate |
| 17 | 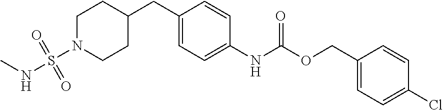 [SMILES: CNS(=O)(=O)N1CCC(Cc2ccc(NC(=O)OCc3ccc(Cl)cc3)cc2)CC1] | 4-chlorobenzyl (4-((1-(N- methylsulfamoyl)piperidin-4- yl)methyl)phenyl)carbamate |
| 18 | 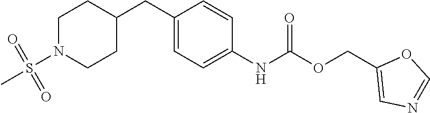 [SMILES: CS(=O)(=O)N1CCC(Cc2ccc(NC(=O)OCc3cnco3)cc2)CC1] | oxazol-5-ylmethyl (4-((1- (methylsulfonyl)piperidin-4- yl)methyl)phenyl)carbamate |
| 19 | 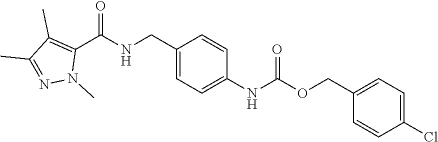 [SMILES: Cc1nn(C)c(C(=O)NCc2ccc(NC(=O)OCc3ccc(Cl)cc3)cc2)c1C] | 4-chlorobenzyl (4- ((1,3,4-trimethyl-1H- pyrazole-5- carboxamido)meth- yl)phenyl)carbamate |
| 20 | 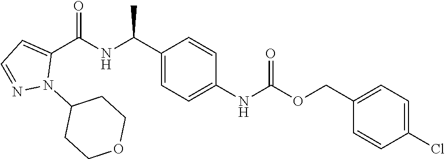 [SMILES: C[C@H](NC(=O)c1ccnn1C1CCOCC1)c1ccc(NC(=O)OCc2ccc(Cl)cc2)cc1] | 4-chlorobenzyl (S)-(4-(1- (1-(tetrahydro-2H-pyran- 4-yl)-1H-pyrazole-5- carboxamido)eth- yl)phenyl)carbamate |
| 21 | 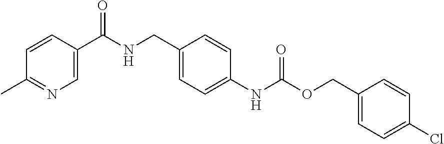 [SMILES: Cc1ccc(C(=O)NCc2ccc(NC(=O)OCc3ccc(Cl)cc3)cc2)cn1] | 4-chlorobenzyl (4-((6- methylnicotinamido)meth- yl)phenyl)carbamate |
| 22 | 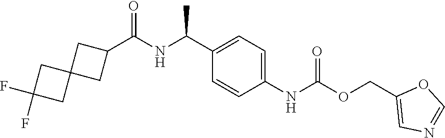 [SMILES: C[C@H](NC(=O)C1CC2(C1)CC(F)(F)C2)c1ccc(NC(=O)OCc2cnco2)cc1] | oxazol-5-ylmethyl (S)- (4-(1-(6,6- difluorospiro[3.3]heptane-2- carboxamido)eth- yl)phenyl)carbamate |
| 23 | 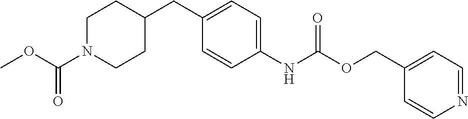 [SMILES: COC(=O)N1CCC(Cc2ccc(NC(=O)OCc3ccncc3)cc2)CC1] | methyl 4-(4-(((pyridin-4- ylmethoxy)carbonyl)ami- no)benzyl)piperidine-1- carboxylate |
| 24 | 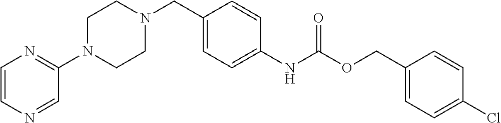 [SMILES: O=C(Nc1ccc(CN2CCN(c3cnccn3)CC2)cc1)OCc1ccc(Cl)cc1] | 4-chlorobenzyl (4-((4- (pyrazin-2-yl)piperazin-1- yl)methyl)phenyl)carbamate |
| 25 | 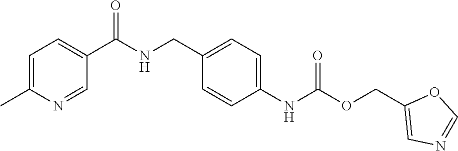 [SMILES: Cc1ccc(C(=O)NCc2ccc(NC(=O)OCc3cnco3)cc2)cn1] | oxazol-5-ylmethyl (4-((6- methylnicotinamido)meth- yl)phenyl)carbamate |
| 26 | 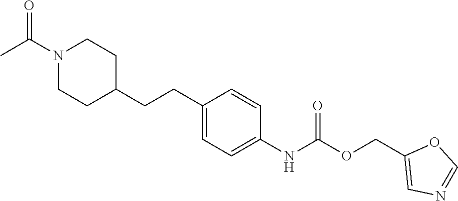 [SMILES: CC(=O)N1CCC(CCc2ccc(NC(=O)OCc3cnco3)cc2)CC1] | oxazol-5-ylmethyl (4-(2- (1-acetylpiperidin-4- yl)ethyl)phenyl)carbamate |
| 27 | 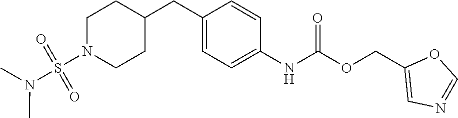 [SMILES: CN(C)S(=O)(=O)N1CCC(Cc2ccc(NC(=O)OCc3cnco3)cc2)CC1] | oxazol-5-ylmethyl (4-((1- (N,N- dimethylsulfamoyl)piperidin-4- yl)methyl)phenyl)carbamate |
| 28 | 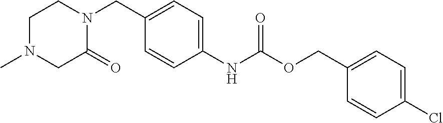 [SMILES: CN1CCN(Cc2ccc(NC(=O)OCc3ccc(Cl)cc3)cc2)C(=O)C1] | 4-chlorobenzyl (4-((4- methyl-2-oxopiperazin-1- yl)methyl)phenyl)carbamate |
| 29 | 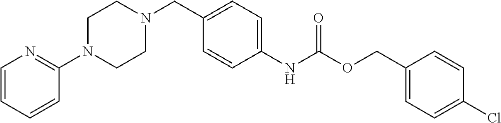 [SMILES: O=C(Nc1ccc(CN2CCN(c3ccccn3)CC2)cc1)OCc1ccc(Cl)cc1] | 4-chlorobenzyl (4-((4- (pyridin-2-yl)piperazin-1- yl)methyl)phenyl)carbamate |
| 30 | 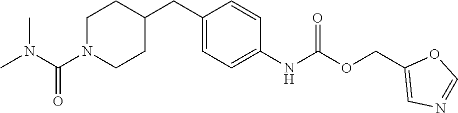 [SMILES: CN(C)C(=O)N1CCC(Cc2ccc(NC(=O)OCc3cnco3)cc2)CC1] | oxazol-5-ylmethyl (4-((1- (dimethylcarbamoyl)piperidin-4- yl)methyl)phenyl)carbamate |
| 31 | 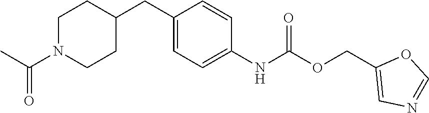 [SMILES: CC(=O)N1CCC(Cc2ccc(NC(=O)OCc3cnco3)cc2)CC1] | oxazol-5-ylmethyl (4-((1- acetylpiperidin-4- yl)methyl)phenyl)carbamate |
| 32 | 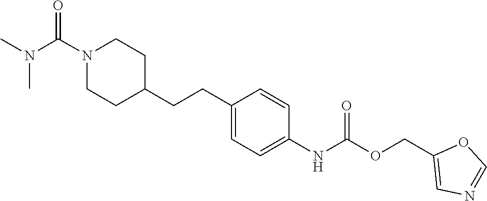 [SMILES: CN(C)C(=O)N1CCC(CCc2ccc(NC(=O)OCc3cnco3)cc2)CC1] | oxazol-5-ylmethyl (4-(2-(1- (dimethylcarbamoyl)piperidin-4- yl)ethyl)phenyl)carbamate |
| 33 | 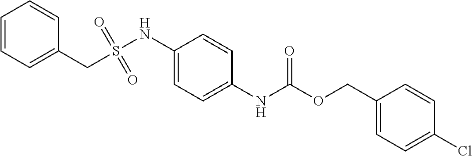 [SMILES: O=C(Nc1ccc(NS(=O)(=O)Cc2ccccc2)cc1)OCc1ccc(Cl)cc1] | 4-chlorobenzyl (4- ((phenylmethyl)sul- fonamido)phenyl)carbamate |
| 34 | 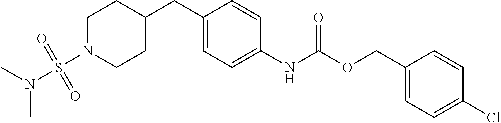 [SMILES: CN(C)S(=O)(=O)N1CCC(Cc2ccc(NC(=O)OCc3ccc(Cl)cc3)cc2)CC1] | 4-chlorobenzyl (4-((1- (N,N- dimethylsulfamoyl)piperidin-4- yl)methyl)phenyl)carbamate |
| 35 | 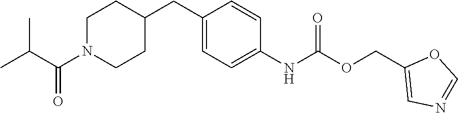 [SMILES: CC(C)C(=O)N1CCC(Cc2ccc(NC(=O)OCc3cnco3)cc2)CC1] | oxazol-5-ylmethyl (4-((1- isobutyrylpiperidin-4- yl)methyl)phenyl)carbamate |
| 36 | 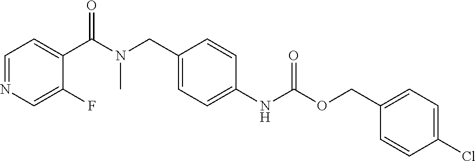 [SMILES: CN(Cc1ccc(NC(=O)OCc2ccc(Cl)cc2)cc1)C(=O)c1ccncc1F] | 4-chlorobenzyl (4-((3- fluoro-N- methylisonicotinamido)meth- yl)phenyl)carbamate |
| 37 | 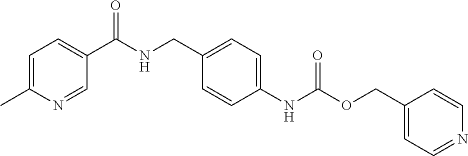 [SMILES: Cc1ccc(C(=O)NCc2ccc(NC(=O)OCc3ccncc3)cc2)cn1] | pyridin-4-ylmethyl (4-((6- methylnicotinamido)meth- yl)phenyl)carbamate |
| 38 | 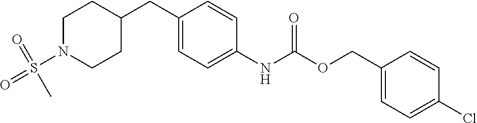 [SMILES: CS(=O)(=O)N1CCC(Cc2ccc(NC(=O)OCc3ccc(Cl)cc3)cc2)CC1] | 4-chlorobenzyl (4-((1- (methylsulfonyl)piperidin-4- yl)methyl)phenyl)carbamate |
| 39 | 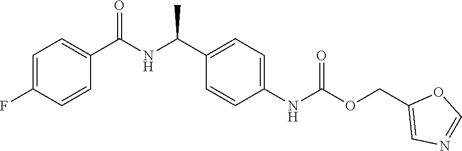 [SMILES: C[C@H](NC(=O)c1ccc(F)cc1)c1ccc(NC(=O)OCc2cnco2)cc1] | oxazol-5-ylmethyl (S)- (4-(1-(4- fluorobenzamido)eth- yl)phenyl)carbamate |
| 40 | 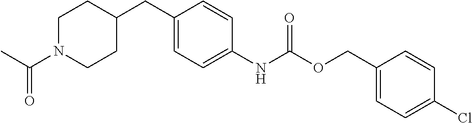 [SMILES: CC(=O)N1CCC(Cc2ccc(NC(=O)OCc3ccc(Cl)cc3)cc2)CC1] | 4-chlorobenzyl (4-((1- acetylpiperidin-4- yl)methyl)phenyl)carbamate |
| 41 | 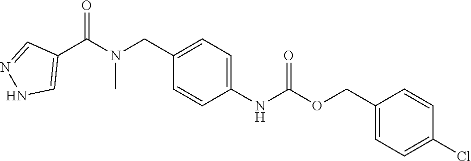 [SMILES: CN(Cc1ccc(NC(=O)OCc2ccc(Cl)cc2)cc1)C(=O)c1cn[nH]c1] | 4-chlorobenzyl (4-((N- methyl-1H-pyrazole-4- carboxamido)meth- yl)phenyl)carbamate |
| 42 | 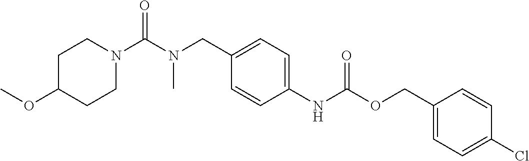 [SMILES: COC1CCN(C(=O)N(C)Cc2ccc(NC(=O)OCc3ccc(Cl)cc3)cc2)CC1] | 4-chlorobenzyl (4-((4- methoxy-N- methylpiperidine-1- carboxamido)meth- yl)phenyl)carbamate |
| 43 | 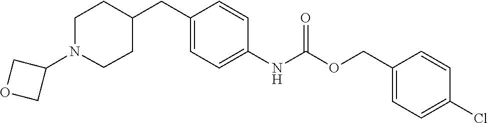 [SMILES: O=C(Nc1ccc(CC2CCN(C3COC3)CC2)cc1)OCc1ccc(Cl)cc1] | 4-chlorobenzyl (4-((1- (oxetan-3-yl)piperidin-4- yl)methyl)phenyl)carbamate |
| 44 | 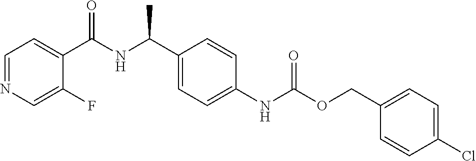 [SMILES: C[C@H](NC(=O)c1ccncc1F)c1ccc(NC(=O)OCc2ccc(Cl)cc2)cc1] | 4-chlorobenzyl (S)-(4-(1-(3- fluoroisonicotinamido)eth- yl)phenyl)carbamate |
| 45 | 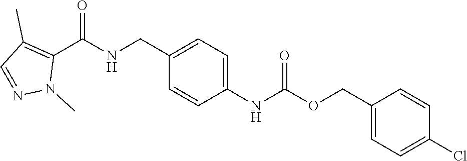 [SMILES: Cc1cnn(C)c1C(=O)NCc1ccc(NC(=O)OCc2ccc(Cl)cc2)cc1] | 4-chlorobenzyl (4-((1,4- dimethyl-1H-pyrazole-5- carboxamido)meth- yl)phenyl)carbamate |
| 46 | 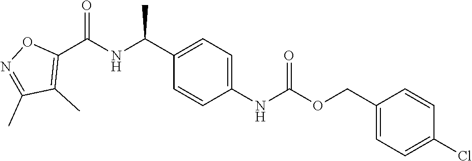 [SMILES: Cc1noc(C(=O)N[C@@H](C)c2ccc(NC(=O)OCc3ccc(Cl)cc3)cc2)c1C] | 4-chlorobenzyl (S)-(4-(1- (3,4-dimethylisoxazole-5- carboxamido)eth- yl)phenyl)carbamate |
| 47 | 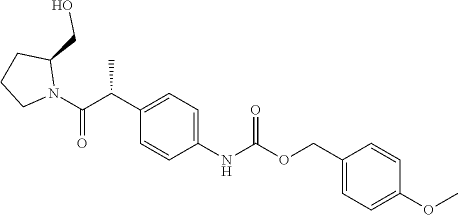 [SMILES: COc1ccc(COC(=O)Nc2ccc([C@@H](C)C(=O)N3CCC[C@H]3CO)cc2)cc1] | 4-methoxybenzyl (4- ((R)-1-((S)-2- (hydroxymethyl)pyrrolidin- 1-yl)-1-oxopropan-2- yl)phenyl)carbamate |
| 48 | 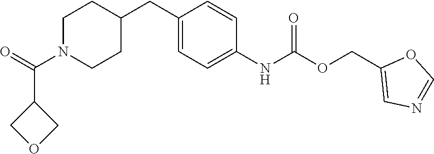 [SMILES: O=C(Nc1ccc(CC2CCN(C(=O)C3COC3)CC2)cc1)OCc1cnco1] | oxazol-5-ylmethyl (4-((1- (oxetane-3- carbonyl)piperidin-4- yl)methyl)phenyl)carbamate |
| 49 | 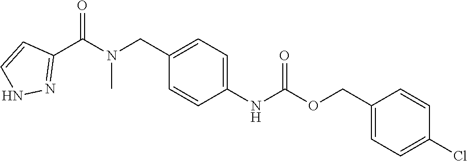 [SMILES: CN(Cc1ccc(NC(=O)OCc2ccc(Cl)cc2)cc1)C(=O)c1cc[nH]n1] | 4-chlorobenzyl (4-((N- methyl-1H-pyrazole-3- carboxamido)meth- yl)phenyl)carbamate |
| 50 | 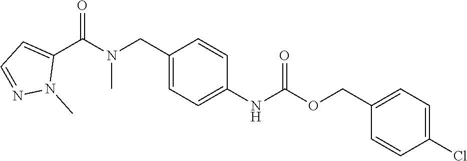 [SMILES: CN(Cc1ccc(NC(=O)OCc2ccc(Cl)cc2)cc1)C(=O)c1ccnn1C] | 4-chlorobenzyl (4-((N,1- dimethyl-1H-pyrazole-5- carboxamido)meth- yl)phenyl)carbamate |
| 51 | 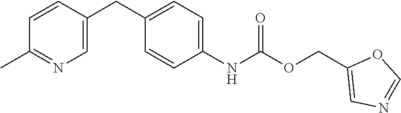 [SMILES: Cc1ccc(Cc2ccc(NC(=O)OCc3cnco3)cc2)cn1] | oxazol-5-ylmethyl (4-((6- methylpyridin-3- yl)methyl)phenyl)carbamate |
| 52 | 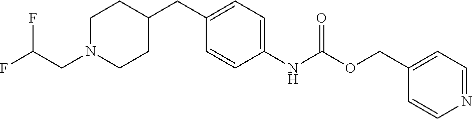 [SMILES: O=C(Nc1ccc(CC2CCN(CC(F)F)CC2)cc1)OCc1ccncc1] | pyridin-4-ylmethyl (4- ((1-(2,2- difluoroethyl)piperidin-4- yl)methyl)phenyl)carbamate |
| 53 | 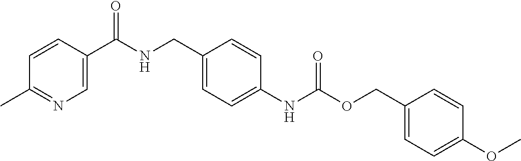 | 4-methoxybenzyl (4-((6- methylnicotinamido)meth- yl)phenyl)carbamate |
| 54 | 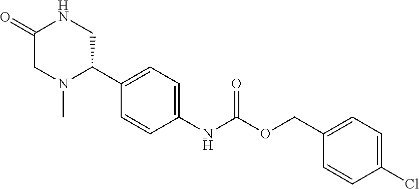 [SMILES: CN1CC(=O)NC[C@@H]1c1ccc(NC(=O)OCc2ccc(Cl)cc2)cc1] | 4-chlorobenzyl (S)-(4-(1- methyl-5-oxopiperazin-2- yl)phenyl)carbamate |
| 55 | 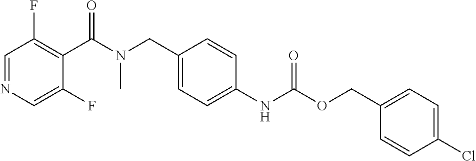 [SMILES: CN(Cc1ccc(NC(=O)OCc2ccc(Cl)cc2)cc1)C(=O)c1c(F)cncc1F] | 4-chlorobenzyl (4-((3,5- difluoro-N- methylisonicotinamido)meth- yl)phenyl)carbamate |
| 56 | 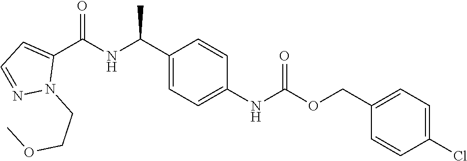 [SMILES: COCCn1nccc1C(=O)N[C@@H](C)c1ccc(NC(=O)OCc2ccc(Cl)cc2)cc1] | 4-chlorobenzyl (S)-(4-(1- (1-(2-methoxyethyl)-1H- pyrazole-5- carboxamido)eth- yl)phenyl)carbamate |
| 57 | 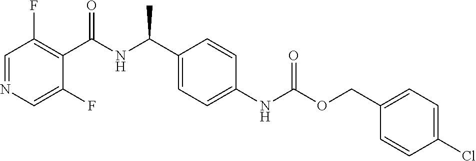 [SMILES: C[C@H](NC(=O)c1c(F)cncc1F)c1ccc(NC(=O)OCc2ccc(Cl)cc2)cc1] | 4-chlorobenzyl (S)-(4-(1- (3,5- difluoroisonicotinamido)eth- yl)phenyl)carbamate |
| 58 | 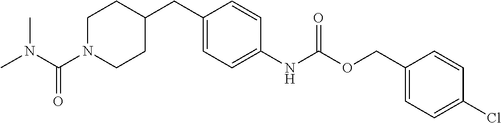 [SMILES: CN(C)C(=O)N1CCC(Cc2ccc(NC(=O)OCc3ccc(Cl)cc3)cc2)CC1] | 4-chlorobenzyl (4-((1- (dimethylcarbamoyl)piperidin-4- yl)methyl)phenyl)carbamate |
| 59 | 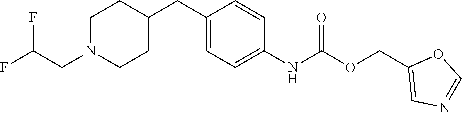 [SMILES: O=C(Nc1ccc(CC2CCN(CC(F)F)CC2)cc1)OCc1cnco1] | oxazol-5-ylmethyl (4-((1- (2,2- difluoroethyl)piperidin-4- yl)methyl)phenyl)carbamate |
| 60 | 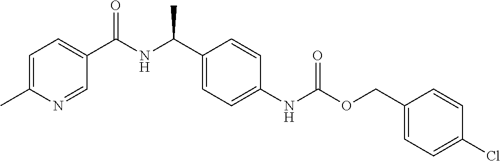 [SMILES: Cc1ccc(C(=O)N[C@@H](C)c2ccc(NC(=O)OCc3ccc(Cl)cc3)cc2)cn1] | 4-chlorobenzyl (S)-(4-(1-(6- methylnicotinamido)eth- yl)phenyl)carbamate |
| 61 | 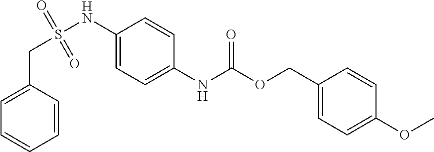 [SMILES: COc1ccc(COC(=O)Nc2ccc(NS(=O)(=O)Cc3ccccc3)cc2)cc1] | 4-methoxybenzyl (4- ((phenylmethyl)sul- fonamido)phenyl)carbamate |
| 62 | 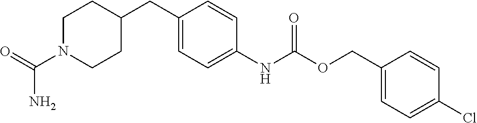 [SMILES: NC(=O)N1CCC(Cc2ccc(NC(=O)OCc3ccc(Cl)cc3)cc2)CC1] | 4-chlorobenzyl (4-((1- carbamoylpiperidin-4- yl)methyl)phenyl)carbamate |
| 63 | 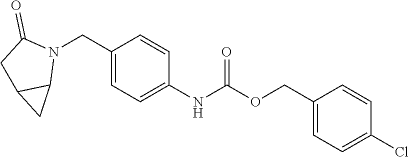 [SMILES: O=C(Nc1ccc(CN2C(=O)CC3CC32)cc1)OCc1ccc(Cl)cc1] | 4-chlorobenzyl (4-((3- oxo-2- azabicyclo[3.1.0]hexan-2- yl)methyl)phenyl)carbamate |
| 64 | 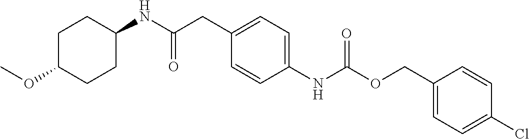 [SMILES: CO[C@H]1CC[C@H](NC(=O)Cc2ccc(NC(=O)OCc3ccc(Cl)cc3)cc2)CC1] | 4-chlorobenzyl (4-(2- (((1r,4r)-4- methoxycyclohexyl)amino)-2- oxoethyl)phenyl)carbamate |
| 65 | 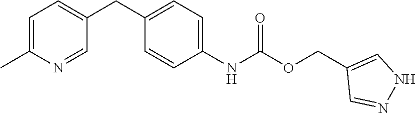 [SMILES: Cc1ccc(Cc2ccc(NC(=O)OCc3cn[nH]c3)cc2)cn1] | (1H-pyrazol-4-yl)methyl (4-((6-methylpyridin-3- yl)methyl)phenyl)carbamate |
| 66 | 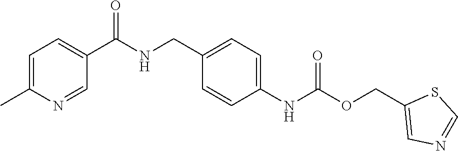 [SMILES: Cc1ccc(C(=O)NCc2ccc(NC(=O)OCc3cncs3)cc2)cn1] | thiazol-5-ylmethyl (4-((6- methylnicotinamido)meth- yl)phenyl)carbamate |
| 67 | 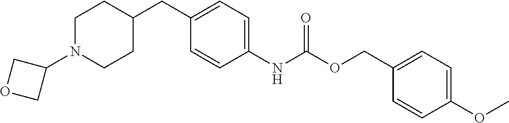 [SMILES: COc1ccc(COC(=O)Nc2ccc(CC3CCN(C4COC4)CC3)cc2)cc1] | 4-methoxybenzyl (4-((1- (oxetan-3-yl)piperidin-4- yl)methyl)phenyl)carbamate |
| 68 | 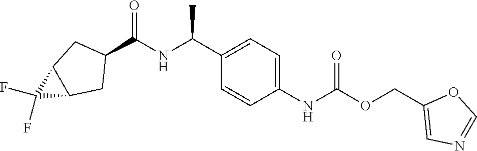 [SMILES: C[C@H](NC(=O)[C@@H]1C[C@@H]2[C@H](C1)C2(F)F)c1ccc(NC(=O)OCc2cnco2)cc1] | oxazol-5-ylmethyl (4- ((S)-1-((1R,3s,5S)-6,6- difluorobicyclo[3.1.0]hexane-3- carboxamido)eth- yl)phenyl)carbamate |
| 69 | 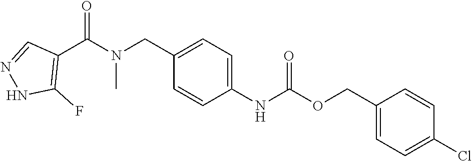 [SMILES: CN(Cc1ccc(NC(=O)OCc2ccc(Cl)cc2)cc1)C(=O)c1cn[nH]c1F] | 4-chlorobenzyl (4-((5- fluoro-N-methyl-1H- pyrazole-4- carboxamido)meth- yl)phenyl)carbamate |
| 70 | 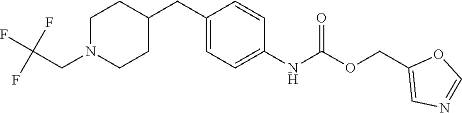 [SMILES: O=C(Nc1ccc(CC2CCN(CC(F)(F)F)CC2)cc1)OCc1cnco1] | oxazol-5-ylmethyl (4-((1- (2,2,2- trifluoroethyl)piperidin-4- yl)methyl)phenyl)carbamate |
| 71 | 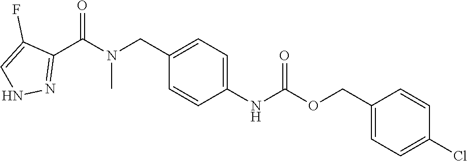 [SMILES: CN(Cc1ccc(NC(=O)OCc2ccc(Cl)cc2)cc1)C(=O)c1n[nH]cc1F] | 4-chlorobenzyl (4-((4- fluoro-N-methyl-1H- pyrazole-3- carboxamido)meth- yl)phenyl)carbamate |
| 72 | 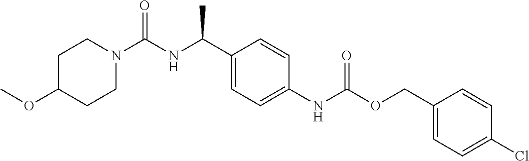 [SMILES: COC1CCN(C(=O)N[C@@H](C)c2ccc(NC(=O)OCc3ccc(Cl)cc3)cc2)CC1] | 4-chlorobenzyl (S)-(4-(1- (4-methoxypiperidine-1- carboxamido)eth- yl)phenyl)carbamate |
| 73 | 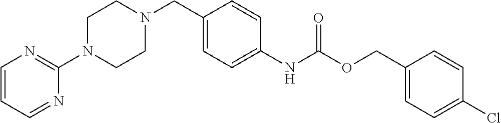 [SMILES: O=C(Nc1ccc(CN2CCN(c3ncccn3)CC2)cc1)OCc1ccc(Cl)cc1] | 4-chlorobenzyl (4-((4- (pyrimidin-2- yl)piperazin-1- yl)methyl)phenyl)carbamate |
| 74 | 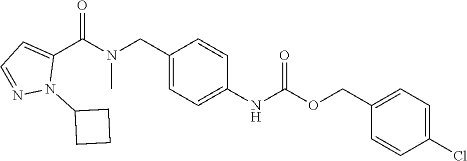 [SMILES: CN(Cc1ccc(NC(=O)OCc2ccc(Cl)cc2)cc1)C(=O)c1ccnn1C1CCC1] | 4-chlorobenzyl (4-((1- cyclobutyl-N-methyl-1H- pyrazole-5- carboxamido)meth- yl)phenyl)carbamate |
| 75 | 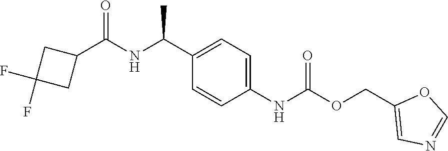 [SMILES: C[C@H](NC(=O)C1CC(F)(F)C1)c1ccc(NC(=O)OCc2cnco2)cc1] | oxazol-5-ylmethyl (S)- (4-(1-(3,3- difluorocyclobutane-1- carboxamido)eth- yl)phenyl)carbamate |
| 76 | 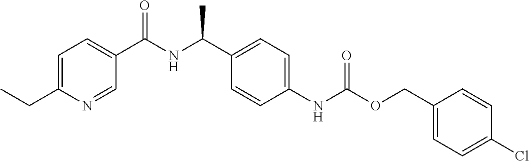 [SMILES: CCc1ccc(C(=O)N[C@@H](C)c2ccc(NC(=O)OCc3ccc(Cl)cc3)cc2)cn1] | 4-chlorobenzyl (S)-(4-(1-(6- ethylnicotinamido)eth- yl)phenyl)carbamate |
| 77 | 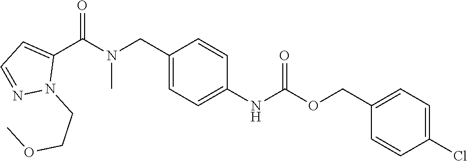 [SMILES: COCCn1nccc1C(=O)N(C)Cc1ccc(NC(=O)OCc2ccc(Cl)cc2)cc1] | 4-chlorobenzyl (4-((1-(2- methoxyethyl)-N-methyl- 1H-pyrazole-5- carboxamido)meth- yl)phenyl)carbamate |
| 78 | 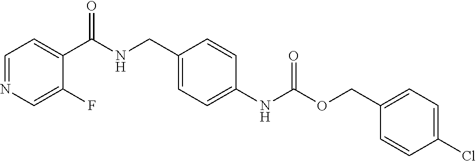 [SMILES: O=C(Nc1ccc(CNC(=O)c2ccncc2F)cc1)OCc1ccc(Cl)cc1] | 4-chlorobenzyl (4-((3- fluoroisonicotinamido)meth- yl)phenyl)carbamate |
| 79 | 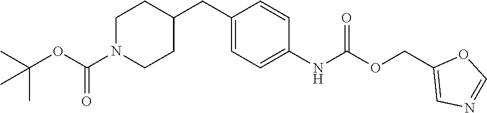 [SMILES: CC(C)(C)OC(=O)N1CCC(Cc2ccc(NC(=O)OCc3cnco3)cc2)CC1] | tert-butyl 4-(4-(((oxazol-5- ylmethoxy)carbonyl)ami- no)benzyl)piperidine-1- carboxylate |
| 80 | 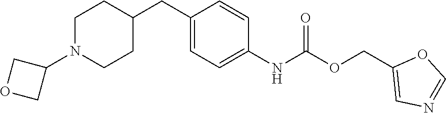 [SMILES: O=C(Nc1ccc(CC2CCN(C3COC3)CC2)cc1)OCc1cnco1] | oxazol-5-ylmethyl (4-((1- (oxetan-3-yl)piperidin-4- yl)methyl)phenyl)carbamate |
| 81 | 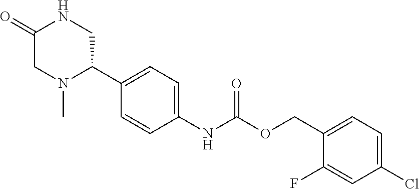 [SMILES: CN1CC(=O)NC[C@@H]1c1ccc(NC(=O)OCc2ccc(Cl)cc2F)cc1] | 4-chloro-2-fluorobenzyl (S)-(4-(1-methyl-5- oxopiperazin-2- yl)phenyl)carbamate |
| 82 | 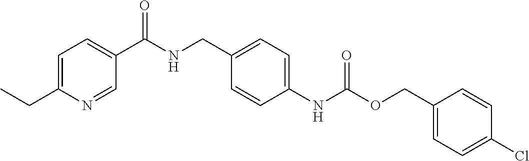 [SMILES: CCc1ccc(C(=O)NCc2ccc(NC(=O)OCc3ccc(Cl)cc3)cc2)cn1] | 4-chlorobenzyl (4-((6- ethylnicotinamido)meth- yl)phenyl)carbamate |
| 83 | 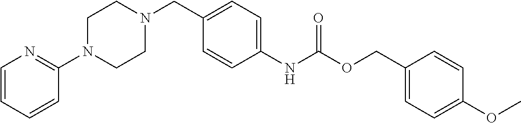 [SMILES: COc1ccc(COC(=O)Nc2ccc(CN3CCN(c4ccccn4)CC3)cc2)cc1] | 4-methoxybenzyl (4-((4- (pyridin-2-yl)piperazin-1- yl)methyl)phenyl)carbamate |
| 84 | 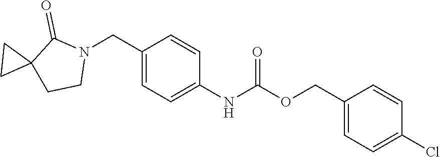 [SMILES: O=C(Nc1ccc(CN2CCC3(CC3)C2=O)cc1)OCc1ccc(Cl)cc1] | 4-chlorobenzyl (4-((4- oxo-5- azaspiro[2.4]heptan-5- yl)methyl)phenyl)carbamate |
| 85 | 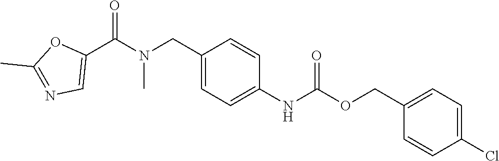 [SMILES: Cc1ncc(C(=O)N(C)Cc2ccc(NC(=O)OCc3ccc(Cl)cc3)cc2)o1] | 4-chlorobenzyl (4-((N,2- dimethyloxazole-5- carboxamido)meth- yl)phenyl)carbamate |
| 86 | 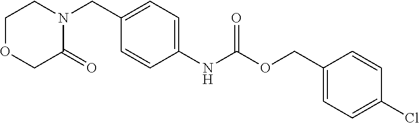 [SMILES: O=C(Nc1ccc(CN2CCOCC2=O)cc1)OCc1ccc(Cl)cc1] | 4-chlorobenzyl (4-((3- oxomorpholino)meth- yl)phenyl)carbamate |
| 87 | 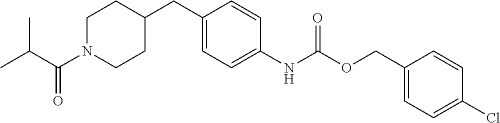 [SMILES: CC(C)C(=O)N1CCC(Cc2ccc(NC(=O)OCc3ccc(Cl)cc3)cc2)CC1] | 4-chlorobenzyl (4-((1- isobutyrylpiperidin-4- yl)methyl)phenyl)carbamate |
| 88 | 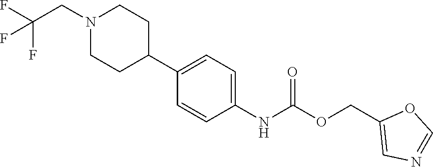 [SMILES: O=C(Nc1ccc(C2CCN(CC(F)(F)F)CC2)cc1)OCc1cnco1] | oxazol-5-ylmethyl (4-(1- (2,2,2- trifluoroethyl)piperidin- 4-yl)phenyl)carbamate |
| 89 | 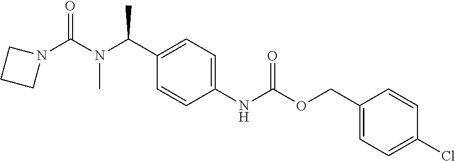 [SMILES: C[C@@H](c1ccc(NC(=O)OCc2ccc(Cl)cc2)cc1)N(C)C(=O)N1CCC1] | 4-chlorobenzyl (S)-(4-(1- (N-methylazetidine-1- carboxamido)eth- yl)phenyl)carbamate |
| 90 | 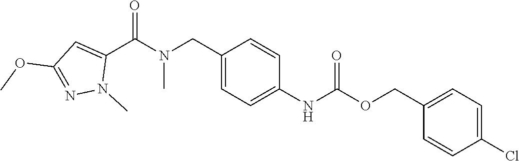 [SMILES: COc1cc(C(=O)N(C)Cc2ccc(NC(=O)OCc3ccc(Cl)cc3)cc2)n(C)n1] | 4-chlorobenzyl (4-((3- methoxy-N,1-dimethyl- 1H-pyrazole-5- carboxamido)meth- yl)phenyl)carbamate |
| 91 | 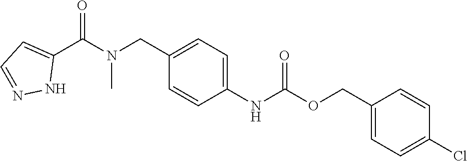 [SMILES: CN(Cc1ccc(NC(=O)OCc2ccc(Cl)cc2)cc1)C(=O)c1ccn[nH]1] | 4-chlorobenzyl (4-((N- methyl-1H-pyrazole-5- carboxamido)meth- yl)phenyl)carbamate |
| 92 | 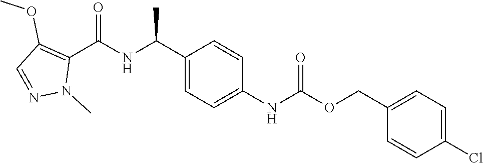 [SMILES: COc1cnn(C)c1C(=O)N[C@@H](C)c1ccc(NC(=O)OCc2ccc(Cl)cc2)cc1] | 4-chlorobenzyl (S)-(4-(1- (4-methoxy-1-methyl- 1H-pyrazole-5- carboxamido)eth- yl)phenyl)carbamate |
| 93 | 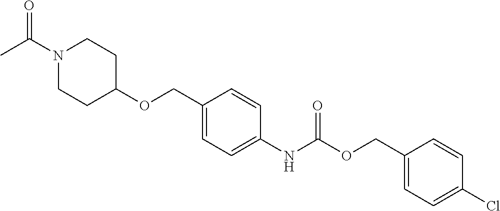 [SMILES: CC(=O)N1CCC(OCc2ccc(NC(=O)OCc3ccc(Cl)cc3)cc2)CC1] | 4-chlorobenzyl (4-(((1- acetylpiperidin-4- yl)oxy)methyl)phenyl)carbamate |
| 94 | 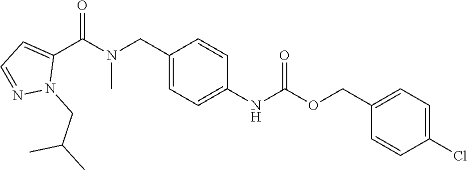 [SMILES: CC(C)Cn1nccc1C(=O)N(C)Cc1ccc(NC(=O)OCc2ccc(Cl)cc2)cc1] | 4-chlorobenzyl (4-((1- isobutyl-N-methyl-1H- pyrazole-5- carboxamido)meth- yl)phenyl)carbamate |
| 95 | 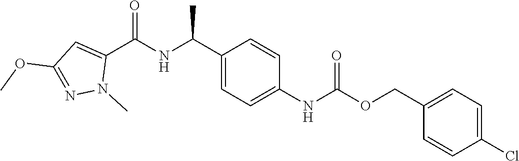 [SMILES: COc1cc(C(=O)N[C@@H](C)c2ccc(NC(=O)OCc3ccc(Cl)cc3)cc2)n(C)n1] | 4-chlorobenzyl (S)-(4-(1- (3-methoxy-1-methyl- 1H-pyrazole-5- carboxamido)eth- yl)phenyl)carbamate |
| 96 | 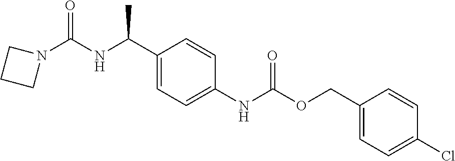 [SMILES: C[C@H](NC(=O)N1CCC1)c1ccc(NC(=O)OCc2ccc(Cl)cc2)cc1] | 4-chlorobenzyl (S)-(4-(1- (azetidine-1- carboxamido)eth- yl)phenyl)carbamate |
| 97 | 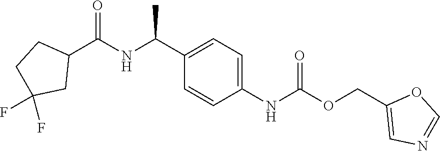 [SMILES: C[C@H](NC(=O)C1CCC(F)(F)C1)c1ccc(NC(=O)OCc2cnco2)cc1] | oxazol-5-ylmethyl (4- ((1S)-1-(3,3- difluorocyclopentane-1- carboxamido)eth- yl)phenyl)carbamate |
| 98 | 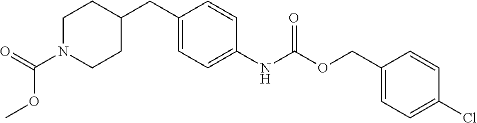 [SMILES: COC(=O)N1CCC(Cc2ccc(NC(=O)OCc3ccc(Cl)cc3)cc2)CC1] | methyl 4-(4-((((4- chlorobenzyl)oxy)car- bonyl)amino)benzyl)piperidine- 1-carboxylate |
| 99 | 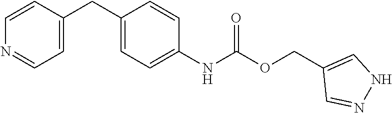 [SMILES: O=C(Nc1ccc(Cc2ccncc2)cc1)OCc1cn[nH]c1] | (1H-pyrazol-4-yl)methyl (4-(pyridin-4- ylmethyl)phenyl)carbamate |
| 100 | 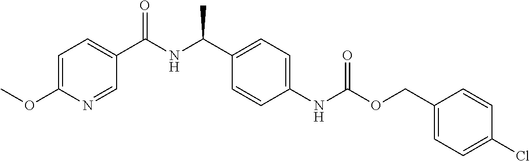 [SMILES: COc1ccc(C(=O)N[C@@H](C)c2ccc(NC(=O)OCc3ccc(Cl)cc3)cc2)cn1] | 4-chlorobenzyl (S)-(4-(1-(6- methoxynicotinamido)eth- yl)phenyl)carbamate |
| 101 | 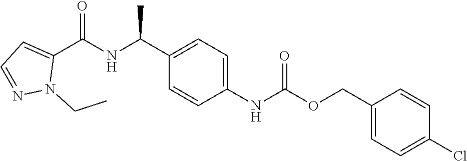 [SMILES: CCn1nccc1C(=O)N[C@@H](C)c1ccc(NC(=O)OCc2ccc(Cl)cc2)cc1] | 4-chlorobenzyl (S)-(4-(1- (1-ethyl-1H-pyrazole-5- carboxamido)eth- yl)phenyl)carbamate |
| 102 | 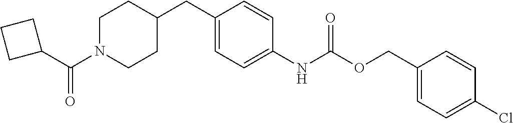 [SMILES: O=C(Nc1ccc(CC2CCN(C(=O)C3CCC3)CC2)cc1)OCc1ccc(Cl)cc1] | 4-chlorobenzyl (4-((1- (cyclobutane- carbonyl)piperidin-4- yl)methyl)phenyl)carbamate |
| 103 | 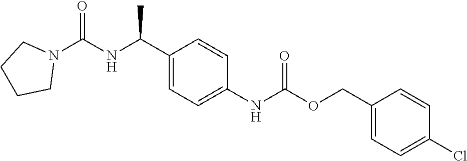 [SMILES: C[C@H](NC(=O)N1CCCC1)c1ccc(NC(=O)OCc2ccc(Cl)cc2)cc1] | 4-chlorobenzyl (S)-(4-(1- (pyrrolidine-1- carboxamido)eth- yl)phenyl)carbamate |
| 104 | 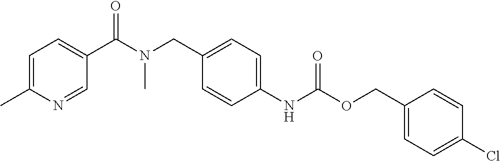 [SMILES: Cc1ccc(C(=O)N(C)Cc2ccc(NC(=O)OCc3ccc(Cl)cc3)cc2)cn1] | 4-chlorobenzyl (4-((N,6- dimethylnicotinamido)meth- yl)phenyl)carbamate |
| 105 | 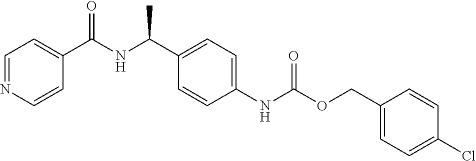 [SMILES: C[C@H](NC(=O)c1ccncc1)c1ccc(NC(=O)OCc2ccc(Cl)cc2)cc1] | 4-chlorobenzyl (S)-(4-(1- (isonicotinamido)eth- yl)phenyl)carbamate |
| 106 | 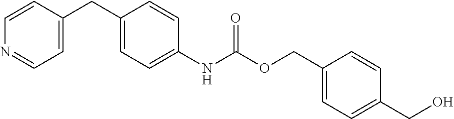 [SMILES: O=C(Nc1ccc(Cc2ccncc2)cc1)OCc1ccc(CO)cc1] | 4-(hydroxymethyl)benzyl (4-(pyridin-4- ylmethyl)phenyl)carbamate |
| 107 | 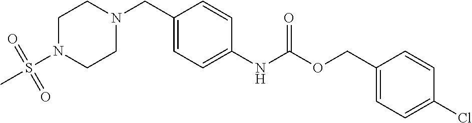 [SMILES: CS(=O)(=O)N1CCN(Cc2ccc(NC(=O)OCc3ccc(Cl)cc3)cc2)CC1] | 4-chlorobenzyl (4-((4- (methylsulfonyl)piperazin-1- yl)methyl)phenyl)carbamate |
| 108 | 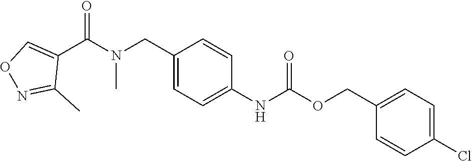 [SMILES: Cc1nocc1C(=O)N(C)Cc1ccc(NC(=O)OCc2ccc(Cl)cc2)cc1] | 4-chlorobenzyl (4-((N,3- dimethylisoxazole-4- carboxamido)meth- yl)phenyl)carbamate |
| 109 | 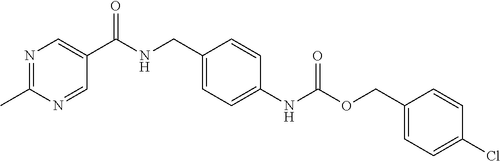 [SMILES: Cc1ncc(C(=O)NCc2ccc(NC(=O)OCc3ccc(Cl)cc3)cc2)cn1] | 4-chlorobenzyl (4-((2- methylpyrimidine-5- carboxamido)meth- yl)phenyl)carbamate |
| 110 | 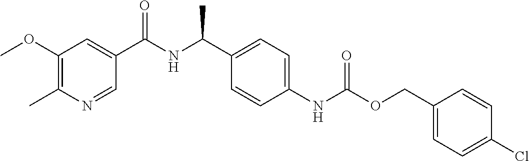 [SMILES: COc1cc(C(=O)N[C@@H](C)c2ccc(NC(=O)OCc3ccc(Cl)cc3)cc2)cnc1C] | 4-chlorobenzyl (S)-(4-(1- (5-methoxy-6- methylnicotinamido)eth- yl)phenyl)carbamate |
| 111 | 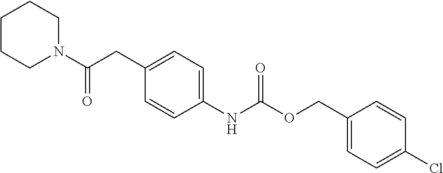 [SMILES: O=C(Nc1ccc(CC(=O)N2CCCCC2)cc1)OCc1ccc(Cl)cc1] | 4-chlorobenzyl (4-(2- oxo-2-(piperidin-1- yl)ethyl)phenyl)carbamate |
| 112 | 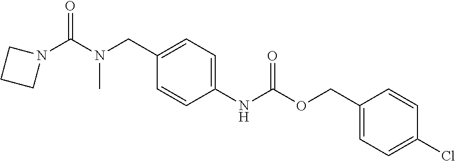 [SMILES: CN(Cc1ccc(NC(=O)OCc2ccc(Cl)cc2)cc1)C(=O)N1CCC1] | 4-chlorobenzyl (4-((N- methylazetidine-1- carboxamido)meth- yl)phenyl)carbamate |
| 113 | 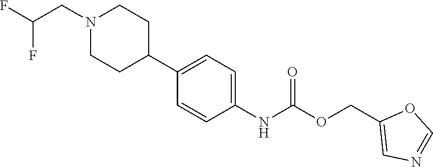 [SMILES: O=C(Nc1ccc(C2CCN(CC(F)F)CC2)cc1)OCc1cnco1] | oxazol-5-ylmethyl (4-(1-(2,2- difluoroethyl)piperidin-4- yl)phenyl)carbamate |
| 114 | 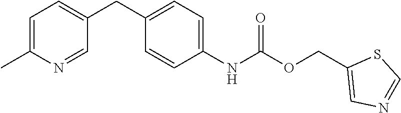 [SMILES: Cc1ccc(Cc2ccc(NC(=O)OCc3cncs3)cc2)cn1] | thiazol-5-ylmethyl (4- ((6-methylpyridin-3- yl)methyl)phenyl)carbamate |
| 115 | 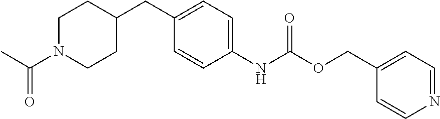 [SMILES: CC(=O)N1CCC(Cc2ccc(NC(=O)OCc3ccncc3)cc2)CC1] | pyridin-4-ylmethyl (4- ((1-acetylpiperidin-4- yl)methyl)phenyl)carbamate |
| 116 | 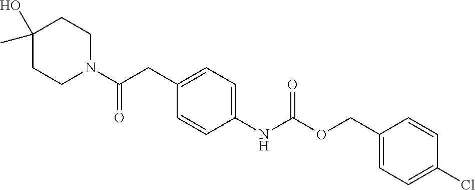 [SMILES: CC1(O)CCN(C(=O)Cc2ccc(NC(=O)OCc3ccc(Cl)cc3)cc2)CC1] | 4-chlorobenzyl (4-(2-(4- hydroxy-4- methylpiperidin-1-yl)-2- oxoethyl)phenyl)carbamate |
| 117 | 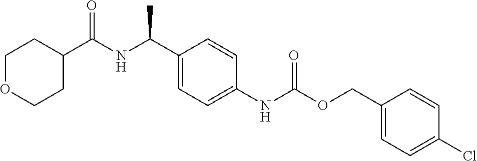 [SMILES: C[C@H](NC(=O)C1CCOCC1)c1ccc(NC(=O)OCc2ccc(Cl)cc2)cc1] | 4-chlorobenzyl (S)-(4-(1- (tetrahydro-2H-pyran-4- carboxamido)eth- yl)phenyl)carbamate |
| 118 | 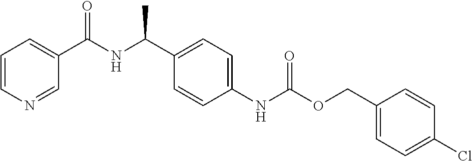 [SMILES: C[C@H](NC(=O)c1cccnc1)c1ccc(NC(=O)OCc2ccc(Cl)cc2)cc1] | 4-chlorobenzyl (S)-(4-(1- (nicotinamido)eth- yl)phenyl)carbamate |
| 119 | 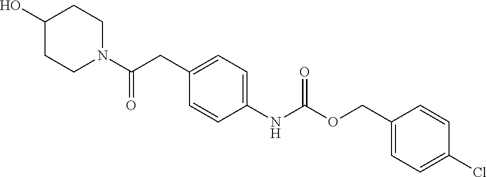 [SMILES: O=C(Nc1ccc(CC(=O)N2CCC(O)CC2)cc1)OCc1ccc(Cl)cc1] | 4-chlorobenzyl (4-(2-(4- hydroxypiperidin-1-yl)-2- oxoethyl)phenyl)carbamate |
| 120 | 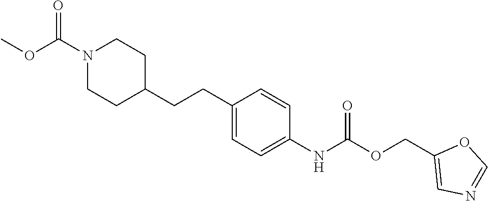 [SMILES: COC(=O)N1CCC(CCc2ccc(NC(=O)OCc3cnco3)cc2)CC1] | methyl 4-(4-(((oxazol-5- ylmethoxy)carbonyl)ami- no)phenethyl)piperidine- 1-carboxylate |
| 121 | 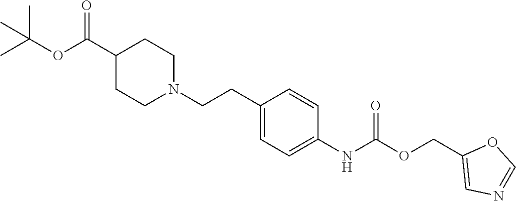 [SMILES: CC(C)(C)OC(=O)C1CCN(CCc2ccc(NC(=O)OCc3cnco3)cc2)CC1] | tert-butyl 4-(4-(((oxazol-5- ylmethoxy)carbonyl)ami- no)phenethyl)piperidine- 1-carboxylate |
| 122 | 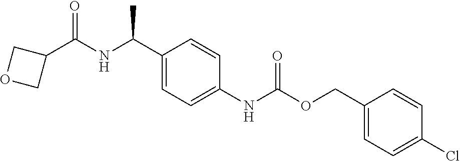 [SMILES: C[C@H](NC(=O)C1COC1)c1ccc(NC(=O)OCc2ccc(Cl)cc2)cc1] | 4-chlorobenzyl (S)-(4-(1- (oxetane-3- carboxamido)eth- yl)phenyl)carbamate |
| 123 | 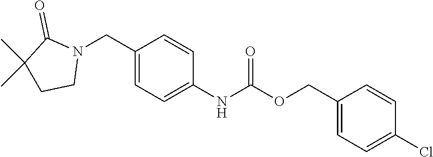 [SMILES: CC1(C)CCN(Cc2ccc(NC(=O)OCc3ccc(Cl)cc3)cc2)C1=O] | 4-chlorobenzyl (4-((3,3- dimethyl-2- oxopyrrolidin-1- yl)methyl)phenyl)carbamate |
| 124 | 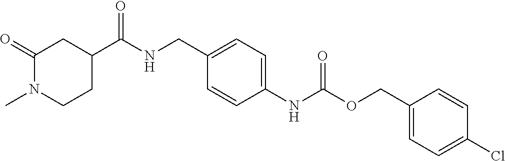 [SMILES: CN1CCC(C(=O)NCc2ccc(NC(=O)OCc3ccc(Cl)cc3)cc2)CC1=O] | 4-chlorobenzyl (4-((1- methyl-2-oxopiperidine-4- carboxamido)meth- yl)phenyl)carbamate |
| 125 | 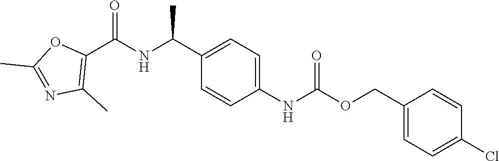 [SMILES: Cc1nc(C)c(C(=O)N[C@@H](C)c2ccc(NC(=O)OCc3ccc(Cl)cc3)cc2)o1] | 4-chlorobenzyl (S)-(4-(1- (2,4-dimethyloxazole-5- carboxamido)eth- yl)phenyl)carbamate |
| 126 | 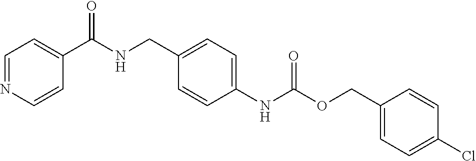 [SMILES: O=C(Nc1ccc(CNC(=O)c2ccncc2)cc1)OCc1ccc(Cl)cc1] | 4-chlorobenzyl (4- (isonicotinamidometh- yl)phenyl)carbamate |
| 127 | 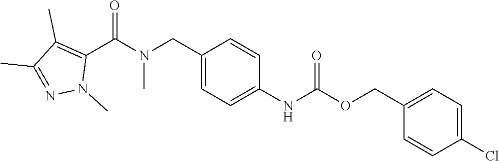 [SMILES: Cc1nn(C)c(C(=O)N(C)Cc2ccc(NC(=O)OCc3ccc(Cl)cc3)cc2)c1C] | 4-chlorobenzyl (4- ((N,1,3,4-tetramethyl- 1H-pyrazole-5- carboxamido)meth- yl)phenyl)carbamate |
| 128 | 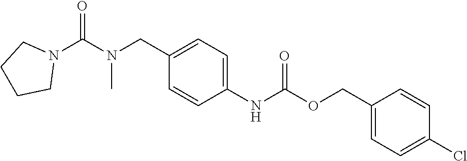 [SMILES: CN(Cc1ccc(NC(=O)OCc2ccc(Cl)cc2)cc1)C(=O)N1CCCC1] | 4-chlorobenzyl (4-((N- methylpyrrolidine-1- carboxamido)meth- yl)phenyl)carbamate |
| 129 | 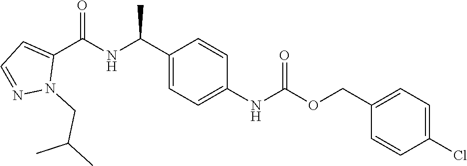 [SMILES: CC(C)Cn1nccc1C(=O)N[C@@H](C)c1ccc(NC(=O)OCc2ccc(Cl)cc2)cc1] | 4-chlorobenzyl (S)-(4-(1- (1-isobutyl-1H-pyrazole-5- carboxamido)eth- yl)phenyl)carbamate |
| 130 | 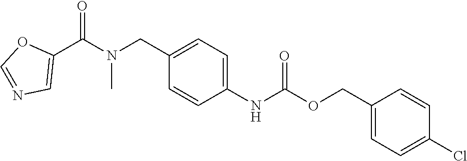 [SMILES: CN(Cc1ccc(NC(=O)OCc2ccc(Cl)cc2)cc1)C(=O)c1cnco1] | 4-chlorobenzyl (4-((N- methyloxazole-5- carboxamido)meth- yl)phenyl)carbamate |
| 131 | 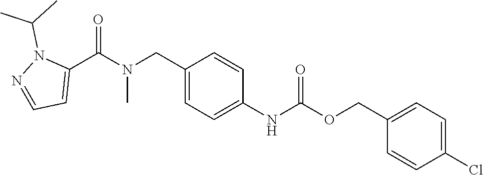 [SMILES: CC(C)n1nccc1C(=O)N(C)Cc1ccc(NC(=O)OCc2ccc(Cl)cc2)cc1] | 4-chlorobenzyl (4-((1- isopropyl-N-methyl-1H- pyrazole-5- carboxamido)meth- yl)phenyl)carbamate |
| 132 | 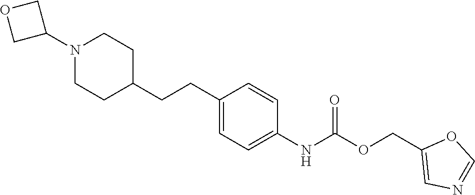 [SMILES: O=C(Nc1ccc(CCC2CCN(C3COC3)CC2)cc1)OCc1cnco1] | oxazol-5-ylmethyl (4-(2- (1-(oxetan-3- yl)piperidin-4- yl)ethyl)phenyl)carbamate |
| 133 | 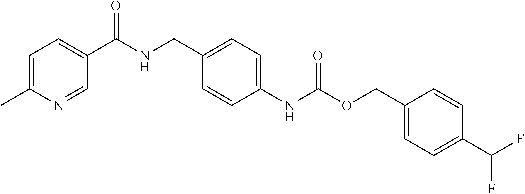 [SMILES: Cc1ccc(C(=O)NCc2ccc(NC(=O)OCc3ccc(C(F)F)cc3)cc2)cn1] | 4-(difluoromethyl)benzyl (4-((6- methylnicotinamido)meth- yl)phenyl)carbamate |
| 134 | 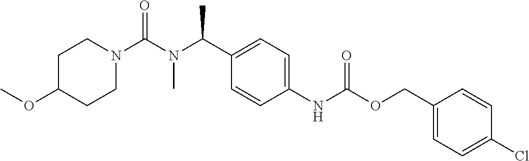 [SMILES: COC1CCN(C(=O)N(C)[C@@H](C)c2ccc(NC(=O)OCc3ccc(Cl)cc3)cc2)CC1] | 4-chlorobenzyl (S)-(4-(1- (4-methoxy-N- methylpiperidine-1- carboxamido)eth- yl)phenyl)carbamate |
| 135 | 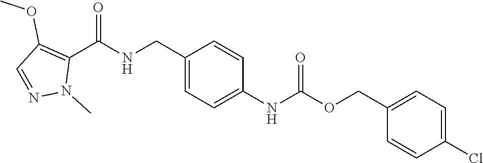 [SMILES: COc1cnn(C)c1C(=O)NCc1ccc(NC(=O)OCc2ccc(Cl)cc2)cc1] | 4-chlorobenzyl (4-((4- methoxy-1-methyl-1H- pyrazole-5- carboxamido)meth- yl)phenyl)carbamate |
| 136 | 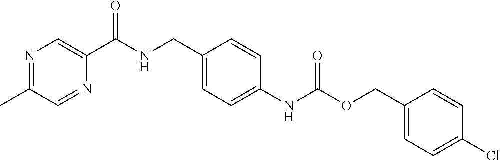 [SMILES: Cc1cnc(C(=O)NCc2ccc(NC(=O)OCc3ccc(Cl)cc3)cc2)cn1] | 4-chlorobenzyl (4-((5- methylpyrazine-2- carboxamido)meth- yl)phenyl)carbamate |
| 137 | 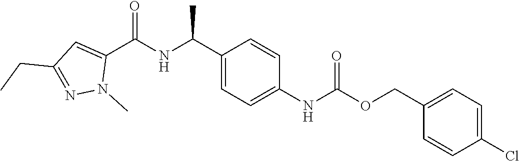 [SMILES: CCc1cc(C(=O)N[C@@H](C)c2ccc(NC(=O)OCc3ccc(Cl)cc3)cc2)n(C)n1] | 4-chlorobenzyl (S)-(4-(1- (3-ethyl-1-methyl-1H- pyrazole-5- carboxamido)eth- yl)phenyl)carbamate |
| 138 | 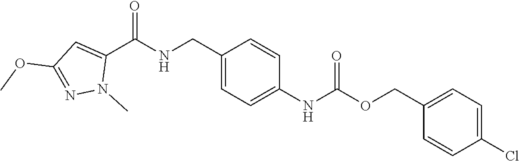 [SMILES: COc1cc(C(=O)NCc2ccc(NC(=O)OCc3ccc(Cl)cc3)cc2)n(C)n1] | 4-chlorobenzyl (4-((3- methoxy-1-methyl-1H- pyrazole-5- carboxamido)meth- yl)phenyl)carbamate |
| 139 | 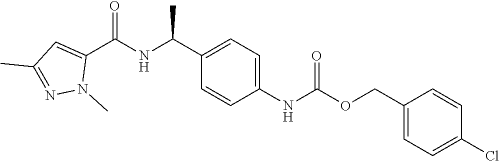 [SMILES: Cc1cc(C(=O)N[C@@H](C)c2ccc(NC(=O)OCc3ccc(Cl)cc3)cc2)n(C)n1] | 4-chlorobenzyl (S)-(4-(1- (1,3-dimethyl-1H- pyrazole-5- carboxamido)eth- yl)phenyl)carbamate |
| 140 | 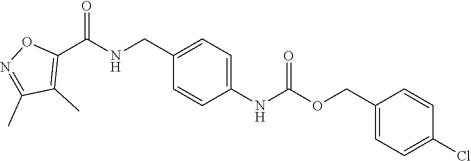 [SMILES: Cc1noc(C(=O)NCc2ccc(NC(=O)OCc3ccc(Cl)cc3)cc2)c1C] | 4-chlorobenzyl (4-((3,4- dimethylisoxazole-5- carboxamido)meth- yl)phenyl)carbamate |
| 141 | 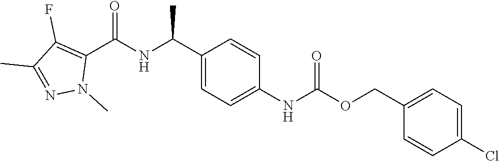 [SMILES: Cc1nn(C)c(C(=O)N[C@@H](C)c2ccc(NC(=O)OCc3ccc(Cl)cc3)cc2)c1F] | 4-chlorobenzyl (S)-(4-(1- (4-fluoro-1,3-dimethyl- 1H-pyrazole-5- carboxamido)eth- yl)phenyl)carbamate |
| 142 | 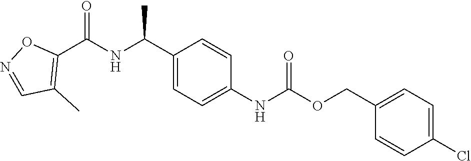 [SMILES: Cc1cnoc1C(=O)N[C@@H](C)c1ccc(NC(=O)OCc2ccc(Cl)cc2)cc1] | 4-chlorobenzyl (S)-(4-(1- (4-methylisoxazole-5- carboxamido)eth- yl)phenyl)carbamate |
| 143 | 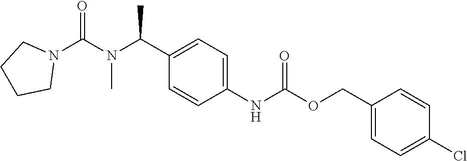 [SMILES: C[C@@H](c1ccc(NC(=O)OCc2ccc(Cl)cc2)cc1)N(C)C(=O)N1CCCC1] | 4-chlorobenzyl (S)-(4-(1- (N-methylpyrrolidine-1- carboxamido)eth- yl)phenyl)carbamate |
| 144 | 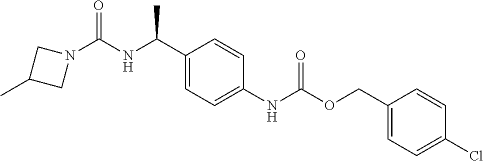 [SMILES: CC1CN(C(=O)N[C@@H](C)c2ccc(NC(=O)OCc3ccc(Cl)cc3)cc2)C1] | 4-chlorobenzyl (S)-(4-(1- (3-methylazetidine-1- carboxamido)eth- yl)phenyl)carbamate |
| 145 | 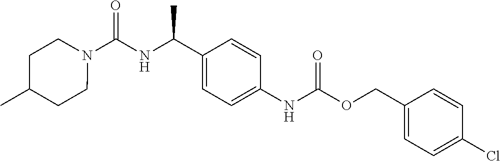 [SMILES: CC1CCN(C(=O)N[C@@H](C)c2ccc(NC(=O)OCc3ccc(Cl)cc3)cc2)CC1] | 4-chlorobenzyl (S)-(4-(1- (4-methylpiperidine-1- carboxamido)eth- yl)phenyl)carbamate |
| 146 | 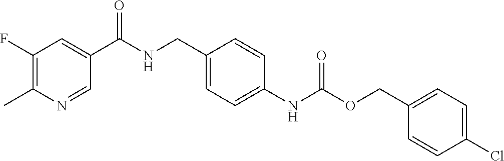 [SMILES: Cc1ncc(C(=O)NCc2ccc(NC(=O)OCc3ccc(Cl)cc3)cc2)cc1F] | 4-chlorobenzyl (4-((5- fluoro-6- methylnicotinamido)meth- yl)phenyl)carbamate |
| 147 | 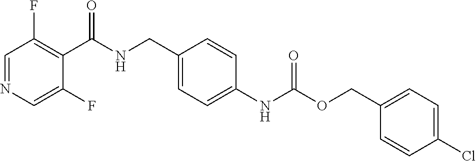 [SMILES: O=C(Nc1ccc(CNC(=O)c2c(F)cncc2F)cc1)OCc1ccc(Cl)cc1] | 4-chlorobenzyl (4-((3,5- difluoroisonicotin- amido)meth- yl)phenyl)carbamate |
| 148 | 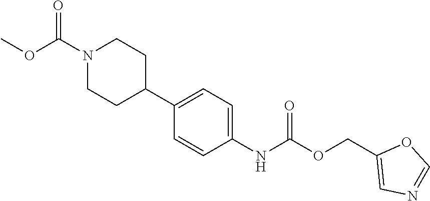 [SMILES: COC(=O)N1CCC(c2ccc(NC(=O)OCc3cnco3)cc2)CC1] | methyl 4-(4-(((oxazol-5- ylmethoxy)carbonyl)ami- no)phenyl)piperidine-1- carboxylate |
| 149 | 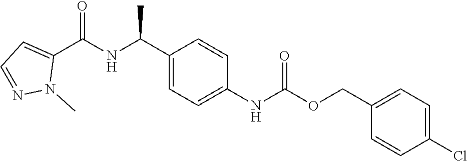 [SMILES: C[C@H](NC(=O)c1ccnn1C)c1ccc(NC(=O)OCc2ccc(Cl)cc2)cc1] | 4-chlorobenzyl (S)-(4-(1- (1-methyl-1H-pyrazole-5- carboxamido)eth- yl)phenyl)carbamate |
| 150 | 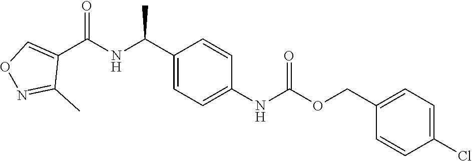 [SMILES: Cc1nocc1C(=O)N[C@@H](C)c1ccc(NC(=O)OCc2ccc(Cl)cc2)cc1] | 4-chlorobenzyl (S)-(4-(1- (3-methylisoxazole-4- carboxamido)eth- yl)phenyl)carbamate |
| 151 | 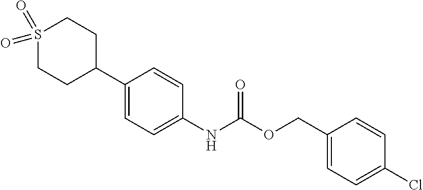 [SMILES: O=C(Nc1ccc(C2CCS(=O)(=O)CC2)cc1)OCc1ccc(Cl)cc1] | 4-chlorobenzyl (4-(1,1- dioxidotetrahydro-2H- thiopyran-4- yl)phenyl)carbamate |
| 152 | 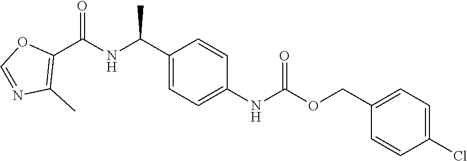 [SMILES: Cc1ncoc1C(=O)N[C@@H](C)c1ccc(NC(=O)OCc2ccc(Cl)cc2)cc1] | 4-chlorobenzyl (S)-(4-(1- (4-methyloxazole-5- carboxamido)eth- yl)phenyl)carbamate |
| 153 | 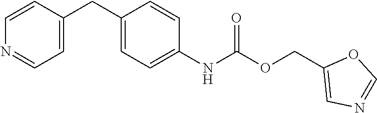 [SMILES: O=C(Nc1ccc(Cc2ccncc2)cc1)OCc1cnco1] | oxazol-5-ylmethyl (4- (pyridin-4- ylmethyl)phenyl)carbamate |
| 154 | 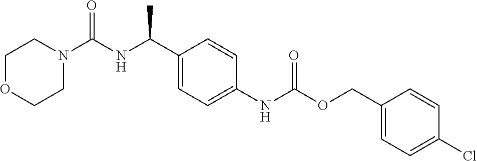 [SMILES: C[C@H](NC(=O)N1CCOCC1)c1ccc(NC(=O)OCc2ccc(Cl)cc2)cc1] | 4-chlorobenzyl (S)-(4-(1- (morpholine-4- carboxamido)eth- yl)phenyl)carbamate |
| 155 | 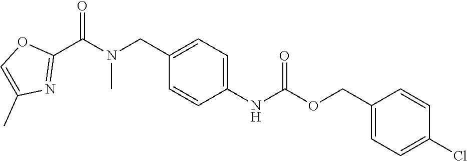 [SMILES: Cc1coc(C(=O)N(C)Cc2ccc(NC(=O)OCc3ccc(Cl)cc3)cc2)n1] | 4-chlorobenzyl (4-((N,4- dimethyloxazole-2- carboxamido)meth- yl)phenyl)carbamate |
| 156 | 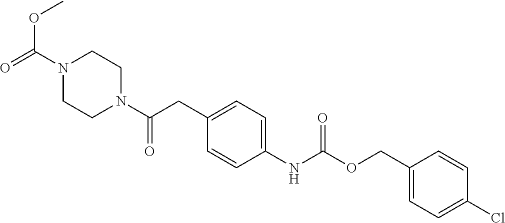 [SMILES: COC(=O)N1CCN(C(=O)Cc2ccc(NC(=O)OCc3ccc(Cl)cc3)cc2)CC1] | methyl 4-(2-(4-((((4- chlorobenzyl)oxy)car- bonyl)amino)phenyl)ace- tyl)piperazine-1-carboxylate |
| 157 | 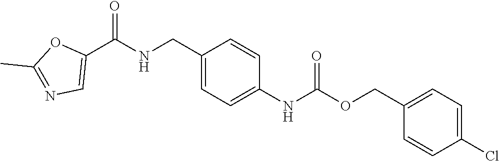 [SMILES: Cc1ncc(C(=O)NCc2ccc(NC(=O)OCc3ccc(Cl)cc3)cc2)o1] | 4-chlorobenzyl (4-((2- methyloxazole-5- carboxamido)meth- yl)phenyl)carbamate |
| 158 | 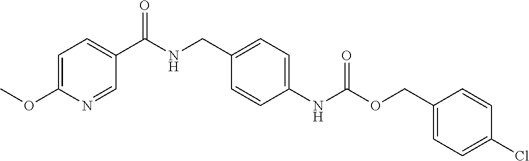 [SMILES: COc1ccc(C(=O)NCc2ccc(NC(=O)OCc3ccc(Cl)cc3)cc2)cn1] | 4-chlorobenzyl (4-((6- methoxynicotinamido)meth- yl)phenyl)carbamate |
| 159 | 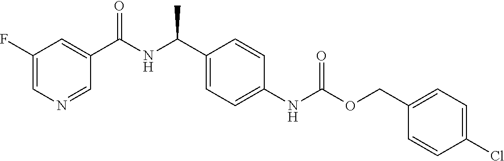 [SMILES: C[C@H](NC(=O)c1cncc(F)c1)c1ccc(NC(=O)OCc2ccc(Cl)cc2)cc1] | 4-chlorobenzyl (S)-(4-(1-(5- fluoronicotinamido)eth- yl)phenyl)carbamate |
| 160 | 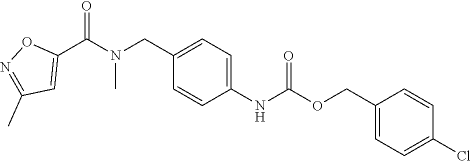 [SMILES: Cc1cc(C(=O)N(C)Cc2ccc(NC(=O)OCc3ccc(Cl)cc3)cc2)on1] | 4-chlorobenzyl (4-((N,3- dimethylisoxazole-5- carboxamido)meth- yl)phenyl)carbamate |
| 161 | 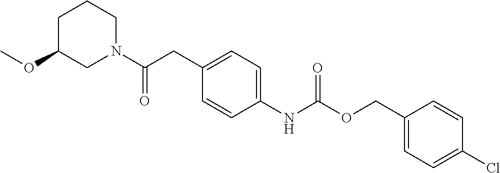 [SMILES: CO[C@H]1CCCN(C(=O)Cc2ccc(NC(=O)OCc3ccc(Cl)cc3)cc2)C1] | 4-chlorobenzyl (S)-(4-(2- (3-methoxypiperidin-1- yl)-2- oxoethyl)phenyl)carbamate |
| 162 | 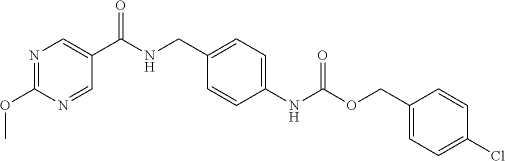 [SMILES: COc1ncc(C(=O)NCc2ccc(NC(=O)OCc3ccc(Cl)cc3)cc2)cn1] | 4-chlorobenzyl (4-((2- methoxypyrimidine-5- carboxamido)meth- yl)phenyl)carbamate |
| 163 | 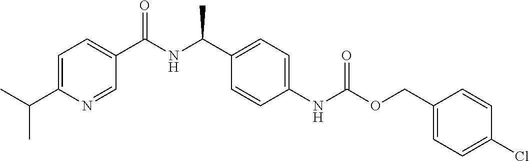 [SMILES: CC(C)c1ccc(C(=O)N[C@@H](C)c2ccc(NC(=O)OCc3ccc(Cl)cc3)cc2)cn1] | 4-chlorobenzyl (S)-(4-(1-(6- isopropylnicotinamido)eth- yl)phenyl)carbamate |
| 164 | 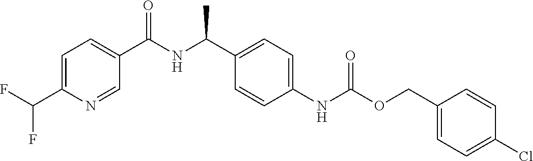 [SMILES: C[C@H](NC(=O)c1ccc(C(F)F)nc1)c1ccc(NC(=O)OCc2ccc(Cl)cc2)cc1] | 4-chlorobenzyl (S)-(4-(1-(6- (difluoromethyl)nicotinami- do)ethyl)phenyl)carbamate |
| 165 | 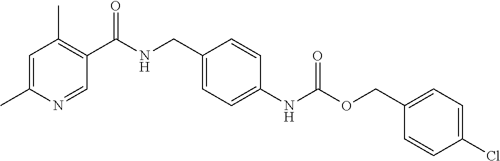 [SMILES: Cc1cc(C)c(C(=O)NCc2ccc(NC(=O)OCc3ccc(Cl)cc3)cc2)cn1] | 4-chlorobenzyl (4-((4,6- dimethylnicotinamido)meth- yl)phenyl)carbamate |
| 166 | 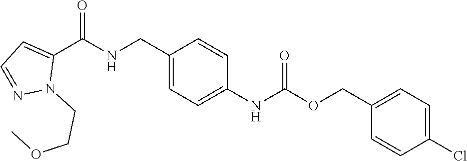 [SMILES: COCCn1nccc1C(=O)NCc1ccc(NC(=O)OCc2ccc(Cl)cc2)cc1] | 4-chlorobenzyl (4-((1-(2- methoxyethyl)-1H- pyrazole-5- carboxamido)meth- yl)phenyl)carbamate |
| 167 | 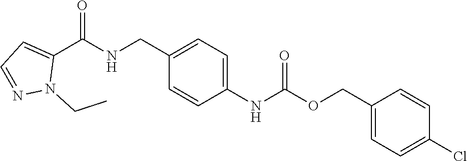 [SMILES: CCn1nccc1C(=O)NCc1ccc(NC(=O)OCc2ccc(Cl)cc2)cc1] | 4-chlorobenzyl (4-((1- ethyl-1H-pyrazole-5- carboxamido)meth- yl)phenyl)carbamate |
| 168 | 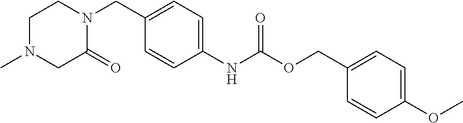 | 4-methoxybenzyl (4-((4- methyl-2-oxopiperazin-1- yl)methyl)phenyl)carbamate |
| 169 | 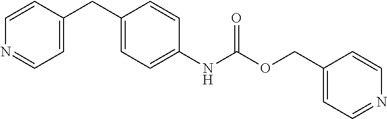 [SMILES: O=C(Nc1ccc(Cc2ccncc2)cc1)OCc1ccncc1] | pyridin-4-ylmethyl (4- (pyridin-4- ylmethyl)phenyl)carbamate |
| 170 | 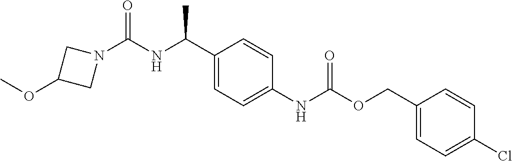 [SMILES: COC1CN(C(=O)N[C@@H](C)c2ccc(NC(=O)OCc3ccc(Cl)cc3)cc2)C1] | 4-chlorobenzyl (S)-(4-(1- (3-methoxyazetidine-1- carboxamido)eth- yl)phenyl)carbamate |
| 171 | 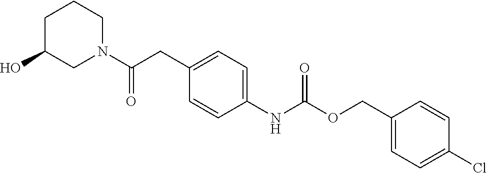 [SMILES: O=C(Nc1ccc(CC(=O)N2CCC[C@H](O)C2)cc1)OCc1ccc(Cl)cc1] | 4-chlorobenzyl (S)-(4-(2- (3-hydroxypiperidin-1- yl)-2- oxoethyl)phenyl)carbamate |
| 172 | 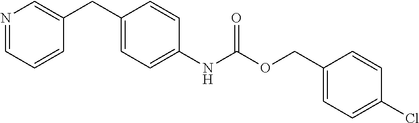 [SMILES: O=C(Nc1ccc(Cc2cccnc2)cc1)OCc1ccc(Cl)cc1] | 4-chlorobenzyl (4- (pyridin-3- ylmethyl)phenyl)carbamate |
| 173 | 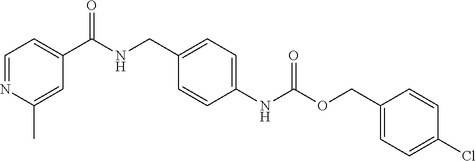 [SMILES: Cc1cc(C(=O)NCc2ccc(NC(=O)OCc3ccc(Cl)cc3)cc2)ccn1] | 4-chlorobenzyl (4-((2- methylisonicotinamido)meth- yl)phenyl)carbamate |
| 174 | 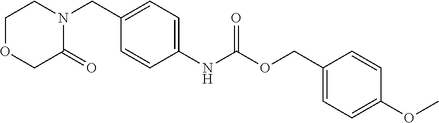 [SMILES: COc1ccc(COC(=O)Nc2ccc(CN3CCOCC3=O)cc2)cc1] | 4-methoxybenzyl (4-((3- oxomorpholino)meth- yl)phenyl)carbamate |
| 175 | 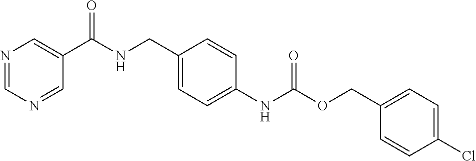 [SMILES: O=C(Nc1ccc(CNC(=O)c2cncnc2)cc1)OCc1ccc(Cl)cc1] | 4-chlorobenzyl (4- ((pyrimidine-5- carboxamido)meth- yl)phenyl)carbamate |
| 176 | 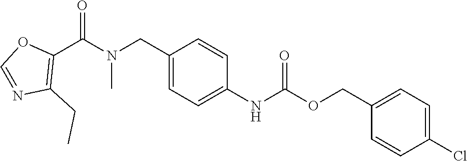 [SMILES: CCc1ncoc1C(=O)N(C)Cc1ccc(NC(=O)OCc2ccc(Cl)cc2)cc1] | 4-chlorobenzyl (4-((4- ethyl-N-methyloxazole-5- carboxamido)meth- yl)phenyl)carbamate |
| 177 | 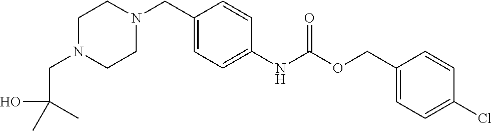 [SMILES: CC(C)(O)CN1CCN(Cc2ccc(NC(=O)OCc3ccc(Cl)cc3)cc2)CC1] | 4-chlorobenzyl (4-((4-(2- hydroxy-2- methylpropyl)piperazin-1- yl)methyl)phenyl)carbamate |
| 178 | 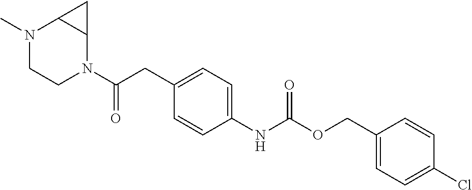 [SMILES: CN1CCN(C(=O)Cc2ccc(NC(=O)OCc3ccc(Cl)cc3)cc2)C2CC21] | 4-chlorobenzyl (4-(2-(5- methyl-2,5- diazabicyclo[4.1.0]heptan- 2-yl)-2- oxoethyl)phenyl)carbamate |
| 179 | 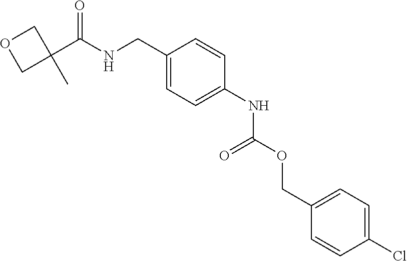 [SMILES: CC1(C(=O)NCc2ccc(NC(=O)OCc3ccc(Cl)cc3)cc2)COC1] | 4-chlorobenzyl (4-((3- methyloxetane-3- carboxamido)meth- yl)phenyl)carbamate |
| 180 | 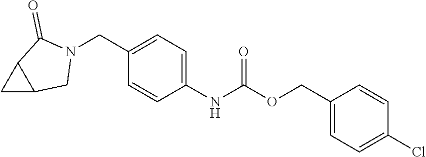 [SMILES: O=C(Nc1ccc(CN2CC3CC3C2=O)cc1)OCc1ccc(Cl)cc1] | 4-chlorobenzyl (4-((2- oxo-3- azabicyclo[3.1.0]hexan-3- yl)methyl)phenyl)carbamate |
| 181 | 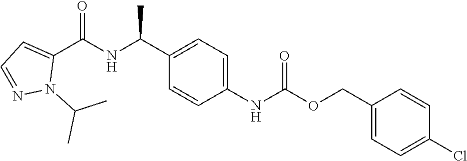 [SMILES: CC(C)n1nccc1C(=O)N[C@@H](C)c1ccc(NC(=O)OCc2ccc(Cl)cc2)cc1] | 4-chlorobenzyl (S)-(4-(1- (1-isopropyl-1H- pyrazole-5- carboxamido)eth- yl)phenyl)carbamate |
| 182 | 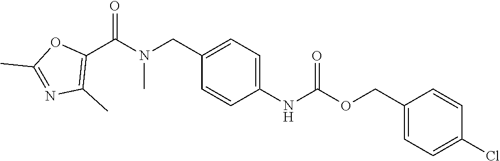 [SMILES: Cc1nc(C)c(C(=O)N(C)Cc2ccc(NC(=O)OCc3ccc(Cl)cc3)cc2)o1] | 4-chlorobenzyl (4- ((N,2,4-trimethyloxazole-5- carboxamido)meth- yl)phenyl)carbamate |
| 183 | 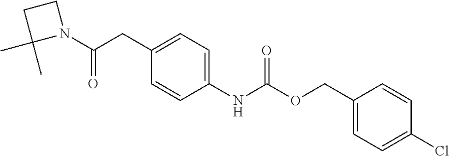 [SMILES: CC1(C)CCN1C(=O)Cc1ccc(NC(=O)OCc2ccc(Cl)cc2)cc1] | 4-chlorobenzyl (4-(2- (2,2-dimethylazetidin-1- yl)-2- oxoethyl)phenyl)carbamate |
| 184 | 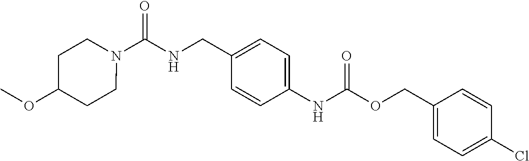 [SMILES: COC1CCN(C(=O)NCc2ccc(NC(=O)OCc3ccc(Cl)cc3)cc2)CC1] | 4-chlorobenzyl (4-((4- methoxypiperidine-1- carboxamido)meth- yl)phenyl)carbamate |
| 185 | 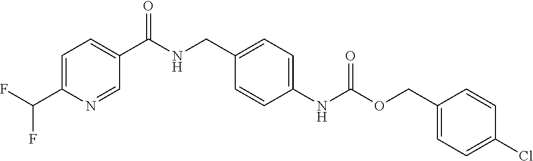 [SMILES: O=C(Nc1ccc(CNC(=O)c2ccc(C(F)F)nc2)cc1)OCc1ccc(Cl)cc1] | 4-chlorobenzyl (4-((6- (difluoromethyl)nicotin- amido)methyl)phenyl)carbamate |
| 186 | 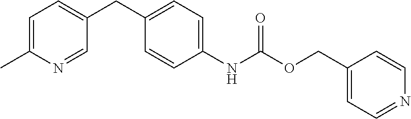 [SMILES: Cc1ccc(Cc2ccc(NC(=O)OCc3ccncc3)cc2)cn1] | pyridin-4-ylmethyl (4- ((6-methylpyridin-3- yl)methyl)phenyl)carbamate |
| 187 | 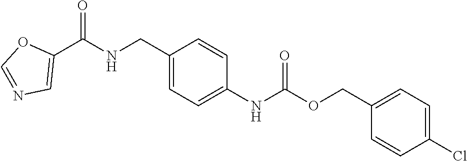 [SMILES: O=C(Nc1ccc(CNC(=O)c2cnco2)cc1)OCc1ccc(Cl)cc1] | 4-chlorobenzyl (4- ((oxazole-5- carboxamido)meth- yl)phenyl)carbamate |
| 188 | 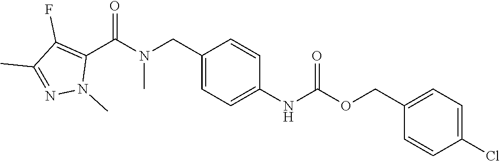 [SMILES: Cc1nn(C)c(C(=O)N(C)Cc2ccc(NC(=O)OCc3ccc(Cl)cc3)cc2)c1F] | 4-chlorobenzyl (4-((4- fluoro-N,1,3-trimethyl- 1H-pyrazole-5- carboxamido)meth- yl)phenyl)carbamate |
| 189 | 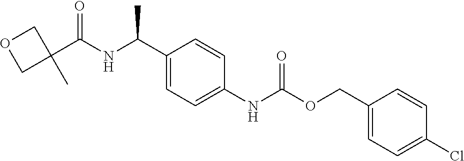 [SMILES: C[C@H](NC(=O)C1(C)COC1)c1ccc(NC(=O)OCc2ccc(Cl)cc2)cc1] | 4-chlorobenzyl (S)-(4-(1- (3-methyloxetane-3- carboxamido)eth- yl)phenyl)carbamate |
| 190 | 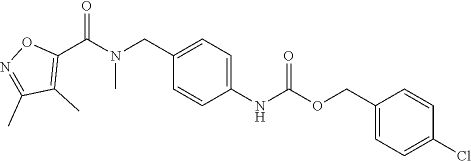 [SMILES: Cc1noc(C(=O)N(C)Cc2ccc(NC(=O)OCc3ccc(Cl)cc3)cc2)c1C] | 4-chlorobenzyl (4- ((N,3,4- trimethylisoxazole-5- carboxamido)meth- yl)phenyl)carbamate |
| 191 | 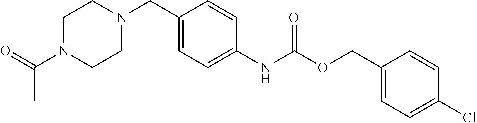 [SMILES: CC(=O)N1CCN(Cc2ccc(NC(=O)OCc3ccc(Cl)cc3)cc2)CC1] | 4-chlorobenzyl (4-((4- acetylpiperazin-1- yl)methyl)phenyl)carbamate |
| 192 | 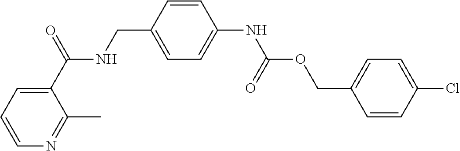 [SMILES: Cc1ncccc1C(=O)NCc1ccc(NC(=O)OCc2ccc(Cl)cc2)cc1] | 4-chlorobenzyl (4-((2- methylnicotinamido)meth- yl)phenyl)carbamate |
| 193 | 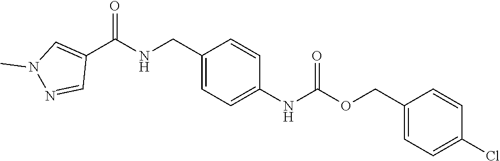 [SMILES: Cn1cc(C(=O)NCc2ccc(NC(=O)OCc3ccc(Cl)cc3)cc2)cn1] | 4-chlorobenzyl (4-((1- methyl-1H-pyrazole-4- carboxamido)meth- yl)phenyl)carbamate |
| 194 | 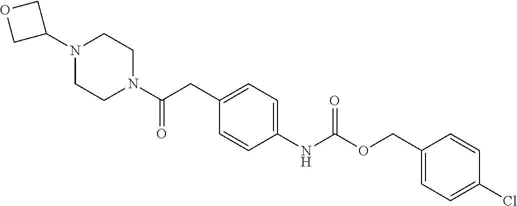 [SMILES: O=C(Nc1ccc(CC(=O)N2CCN(C3COC3)CC2)cc1)OCc1ccc(Cl)cc1] | 4-chlorobenzyl (4-(2-(4- (oxetan-3-yl)piperazin-1- yl)-2- oxoethyl)phenyl)carbamate |
| 195 | 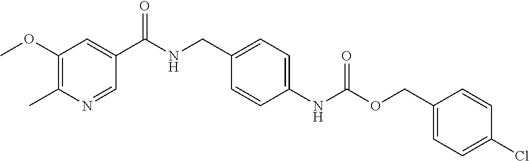 [SMILES: COc1cc(C(=O)NCc2ccc(NC(=O)OCc3ccc(Cl)cc3)cc2)cnc1C] | 4-chlorobenzyl (4-((5- methoxy-6- methylnicotinamido)meth- yl)phenyl)carbamate |
| 196 | 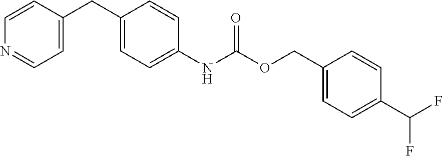 [SMILES: O=C(Nc1ccc(Cc2ccncc2)cc1)OCc1ccc(C(F)F)cc1] | 4-(difluoromethyl)benzyl (4-(pyridin-4- ylmethyl)phenyl)carbamate |
| 197 | 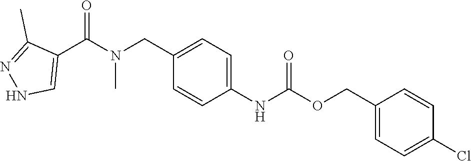 [SMILES: Cc1n[nH]cc1C(=O)N(C)Cc1ccc(NC(=O)OCc2ccc(Cl)cc2)cc1] | 4-chlorobenzyl (4-((N,3- dimethyl-1H-pyrazole-4- carboxamido)meth- yl)phenyl)carbamate |
| 198 | 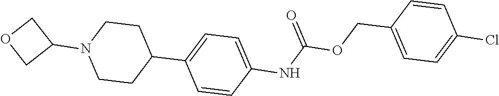 [SMILES: O=C(Nc1ccc(C2CCN(C3COC3)CC2)cc1)OCc1ccc(Cl)cc1] | 4-chlorobenzyl (4-(1- (oxetan-3-yl)piperidin-4- yl)phenyl)carbamate |
| 199 | 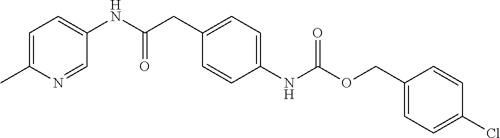 [SMILES: Cc1ccc(NC(=O)Cc2ccc(NC(=O)OCc3ccc(Cl)cc3)cc2)cn1] | 4-chlorobenzyl (4-(2-((6- methylpyridin-3- yl)amino)-2- oxoethyl)phenyl)carbamate |
| 200 | 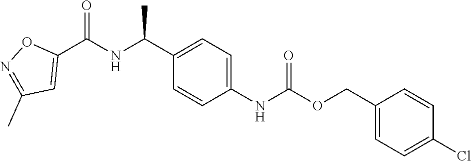 [SMILES: Cc1cc(C(=O)N[C@@H](C)c2ccc(NC(=O)OCc3ccc(Cl)cc3)cc2)on1] | 4-chlorobenzyl (S)-(4-(1- (3-methylisoxazole-5- carboxamido)eth- yl)phenyl)carbamate |
| 201 | 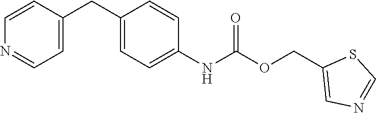 [SMILES: O=C(Nc1ccc(Cc2ccncc2)cc1)OCc1cncs1] | thiazol-5-ylmethyl (4- (pyridin-4- ylmethyl)phenyl)carbamate |
| 202 | 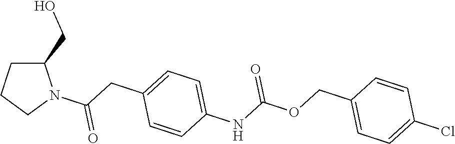 [SMILES: O=C(Nc1ccc(CC(=O)N2CCC[C@H]2CO)cc1)OCc1ccc(Cl)cc1] | 4-chlorobenzyl (S)-(4-(2-(2- (hydroxymethyl)pyrrolidin- 1-yl)-2- oxoethyl)phenyl)carbamate |
| 203 | 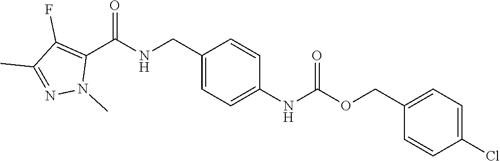 [SMILES: Cc1nn(C)c(C(=O)NCc2ccc(NC(=O)OCc3ccc(Cl)cc3)cc2)c1F] | 4-chlorobenzyl (4-((4- fluoro-1,3-dimethyl-1H- pyrazole-5- carboxamido)meth- yl)phenyl)carbamate |
| 204 | 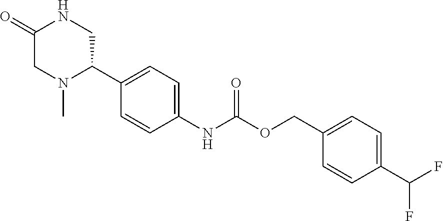 [SMILES: CN1CC(=O)NC[C@@H]1c1ccc(NC(=O)OCc2ccc(C(F)F)cc2)cc1] | 4-(difluoromethyl)benzyl (S)-(4-(1-methyl-5- oxopiperazin-2- yl)phenyl)carbamate |
| 205 | 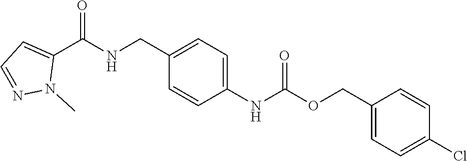 [SMILES: Cn1nccc1C(=O)NCc1ccc(NC(=O)OCc2ccc(Cl)cc2)cc1] | 4-chlorobenzyl (4-((1- methyl-1H-pyrazole-5- carboxamido)meth- yl)phenyl)carbamate |
| 206 | 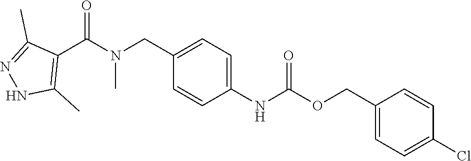 [SMILES: Cc1n[nH]c(C)c1C(=O)N(C)Cc1ccc(NC(=O)OCc2ccc(Cl)cc2)cc1] | 4-chlorobenzyl (4- ((N,3,5-trimethyl-1H- pyrazole-4- carboxamido)meth- yl)phenyl)carbamate |
| 207 | 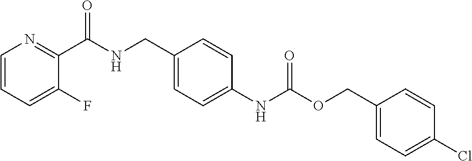 [SMILES: O=C(Nc1ccc(CNC(=O)c2ncccc2F)cc1)OCc1ccc(Cl)cc1] | 4-chlorobenzyl (4-((3- fluoropicolinamido)meth- yl)phenyl)carbamate |
| 208 | 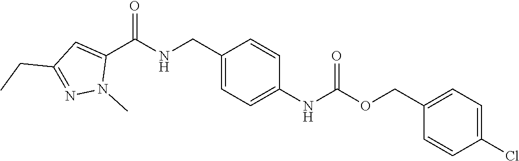 [SMILES: CCc1cc(C(=O)NCc2ccc(NC(=O)OCc3ccc(Cl)cc3)cc2)n(C)n1] | 4-chlorobenzyl (4-((3- ethyl-1-methyl-1H- pyrazole-5- carboxamido)meth- yl)phenyl)carbamate |
| 209 | 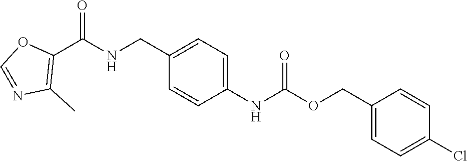 [SMILES: Cc1ncoc1C(=O)NCc1ccc(NC(=O)OCc2ccc(Cl)cc2)cc1] | 4-chlorobenzyl (4-((4- methyloxazole-5- carboxamido)meth- yl)phenyl)carbamate |
| 210 | 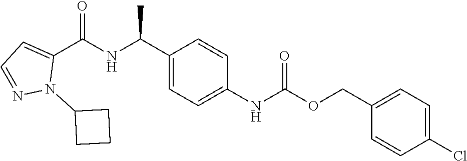 [SMILES: C[C@H](NC(=O)c1ccnn1C1CCC1)c1ccc(NC(=O)OCc2ccc(Cl)cc2)cc1] | 4-chlorobenzyl (S)-(4-(1- (1-cyclobutyl-1H- pyrazole-5- carboxamido)eth- yl)phenyl)carbamate |
| 211 | 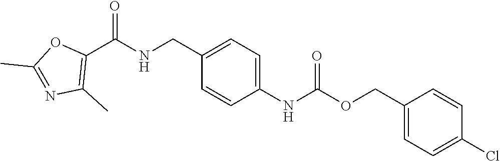 [SMILES: Cc1nc(C)c(C(=O)NCc2ccc(NC(=O)OCc3ccc(Cl)cc3)cc2)o1] | 4-chlorobenzyl (4-((2,4- dimethyloxazole-5- carboxamido)meth- yl)phenyl)carbamate |
| 212 | 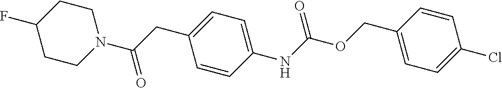 [SMILES: O=C(Nc1ccc(CC(=O)N2CCC(F)CC2)cc1)OCc1ccc(Cl)cc1] | 4-chlorobenzyl (4-(2-(4- fluoropiperidin-1-yl)-2- oxoethyl)phenyl)carbamate |
| 213 | 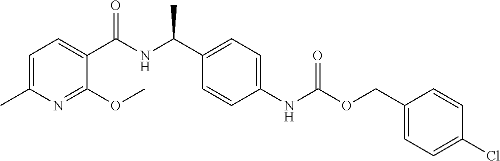 [SMILES: COc1nc(C)ccc1C(=O)N[C@@H](C)c1ccc(NC(=O)OCc2ccc(Cl)cc2)cc1] | 4-chlorobenzyl (S)-(4-(1- (2-methoxy-6- methylnicotinamido)eth- yl)phenyl)carbamate |
| 214 | 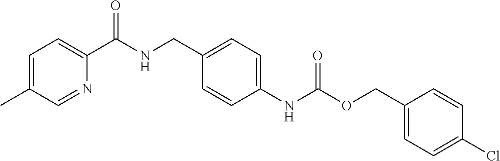 [SMILES: Cc1ccc(C(=O)NCc2ccc(NC(=O)OCc3ccc(Cl)cc3)cc2)nc1] | 4-chlorobenzyl (4-((5- methylpicolinamido)meth- yl)phenyl)carbamate |
| 215 | 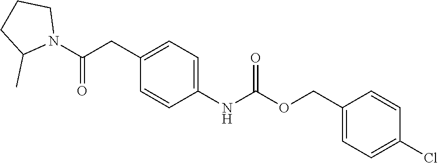 [SMILES: CC1CCCN1C(=O)Cc1ccc(NC(=O)OCc2ccc(Cl)cc2)cc1] | 4-chlorobenzyl (4-(2-(2- methylpyrrolidin-1-yl)-2- oxoethyl)phenyl)carbamate |
| 216 | 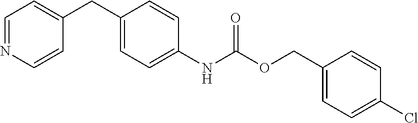 [SMILES: O=C(Nc1ccc(Cc2ccncc2)cc1)OCc1ccc(Cl)cc1] | 4-chlorobenzyl (4- (pyridin-4- ylmethyl)phenyl)carbamate |
| 217 | 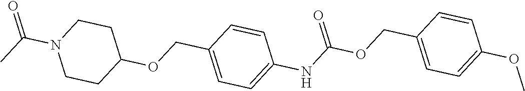 [SMILES: COc1ccc(COC(=O)Nc2ccc(COC3CCN(C(C)=O)CC3)cc2)cc1] | 4-methoxybenzyl (4-(((1- acetylpiperidin-4- yl)oxy)meth- yl)phenyl)carbamate |
| 218 | 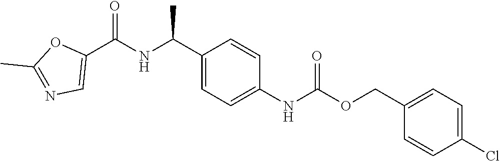 [SMILES: Cc1ncc(C(=O)N[C@@H](C)c2ccc(NC(=O)OCc3ccc(Cl)cc3)cc2)o1] | 4-chlorobenzyl (S)-(4-(1- (2-methyloxazole-5- carboxamido)eth- yl)phenyl)carbamate |
| 219 | 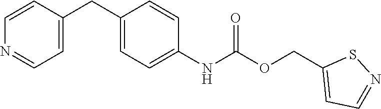 [SMILES: O=C(Nc1ccc(Cc2ccncc2)cc1)OCc1ccns1] | isothiazol-5-ylmethyl (4- (pyridin-4- ylmethyl)phenyl)carbamate |
| 220 | 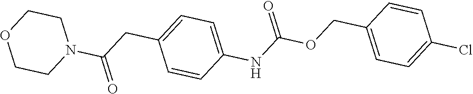 [SMILES: O=C(Nc1ccc(CC(=O)N2CCOCC2)cc1)OCc1ccc(Cl)cc1] | 4-chlorobenzyl (4-(2- morpholino-2- oxoethyl)phenyl)carbamate |
| 221 | 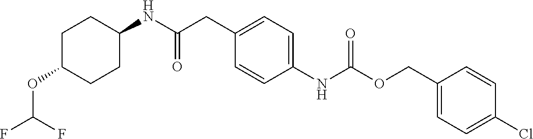 [SMILES: O=C(Cc1ccc(NC(=O)OCc2ccc(Cl)cc2)cc1)N[C@H]1CC[C@H](OC(F)F)CC1] | 4-chlorobenzyl (4-(2- (((1r,4r)-4- (difluoromethoxy)cyclo- hexyl)amino)-2- oxoethyl)phenyl)carbamate |
| 222 | 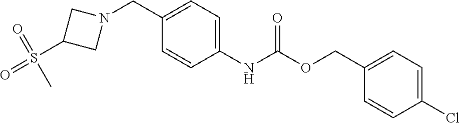 [SMILES: CS(=O)(=O)C1CN(Cc2ccc(NC(=O)OCc3ccc(Cl)cc3)cc2)C1] | 4-chlorobenzyl (4-((3- (methylsulfonyl)azetidin-1- yl)methyl)phenyl)carbamate |
| 223 | 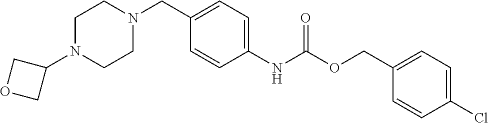 [SMILES: O=C(Nc1ccc(CN2CCN(C3COC3)CC2)cc1)OCc1ccc(Cl)cc1] | 4-chlorobenzyl (4-((4- (oxetan-3-yl)piperazin-1- yl)methyl)phenyl)carbamate |
| 224 | 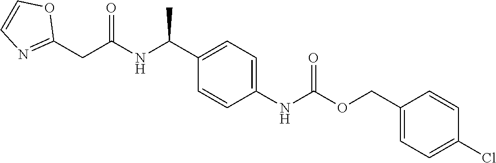 [SMILES: C[C@H](NC(=O)Cc1ncco1)c1ccc(NC(=O)OCc2ccc(Cl)cc2)cc1] | 4-chlorobenzyl (S)-(4-(1- (2-(oxazol-2- yl)acetamido)eth- yl)phenyl)carbamate |
| 225 | 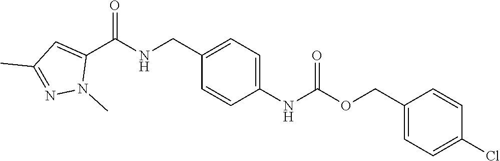 [SMILES: Cc1cc(C(=O)NCc2ccc(NC(=O)OCc3ccc(Cl)cc3)cc2)n(C)n1] | 4-chlorobenzyl (4-((1,3- dimethyl-1H-pyrazole-5- carboxamido)meth- yl)phenyl)carbamate |
| 226 | 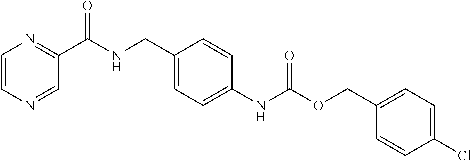 [SMILES: O=C(Nc1ccc(CNC(=O)c2cnccn2)cc1)OCc1ccc(Cl)cc1] | 4-chlorobenzyl (4- ((pyrazine-2- carboxamido)meth- yl)phenyl)carbamate |
| 227 | 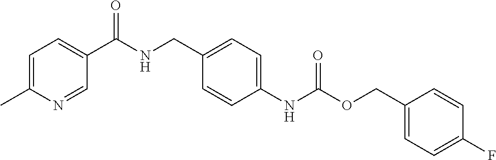 [SMILES: Cc1ccc(C(=O)NCc2ccc(NC(=O)OCc3ccc(F)cc3)cc2)cn1] | 4-fluorobenzyl (4-((6- methylnicotinamido)meth- yl)phenyl)carbamate |
| 228 | 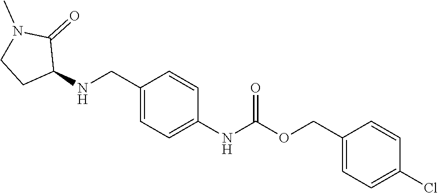 [SMILES: CN1CC[C@H](NCc2ccc(NC(=O)OCc3ccc(Cl)cc3)cc2)C1=O] | 4-chlorobenzyl (S)-(4- (((1-methyl-2- oxopyrrolidin-3- yl)amino)meth- yl)phenyl)carbamate |
| 229 | 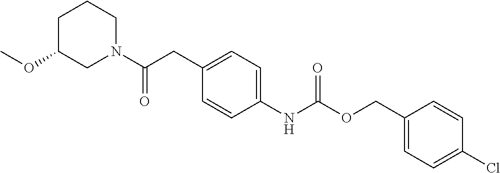 [SMILES: CO[C@@H]1CCCN(C(=O)Cc2ccc(NC(=O)OCc3ccc(Cl)cc3)cc2)C1] | 4-chlorobenzyl (R)-(4-(2- (3-methoxypiperidin-1-yl)-2- oxoethyl)phenyl)carbamate |
| 230 | 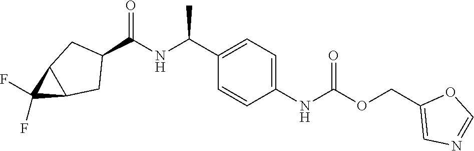 [SMILES: C[C@H](NC(=O)[C@H]1C[C@@H]2[C@H](C1)C2(F)F)c1ccc(NC(=O)OCc2cnco2)cc1] | oxazol-5-ylmethyl (4- ((S)-1-((1R,3r,5S)-6,6- difluorobicyclo[3.1.0]hexane-3- carboxamido)eth- yl)phenyl)carbamate |
| 231 | 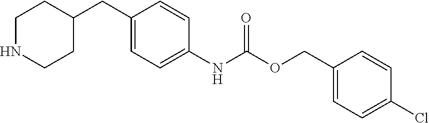 [SMILES: O=C(Nc1ccc(CC2CCNCC2)cc1)OCc1ccc(Cl)cc1] | 4-chlorobenzyl (4- (piperidin-4- ylmethyl)phenyl)carbamate |
| 232 | 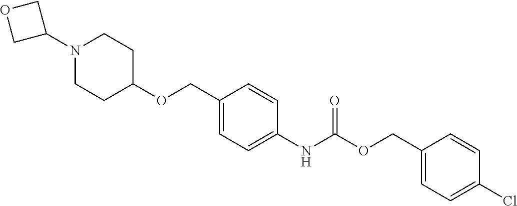 [SMILES: O=C(Nc1ccc(COC2CCN(C3COC3)CC2)cc1)OCc1ccc(Cl)cc1] | 4-chlorobenzyl (4-(((1- (oxetan-3-yl)piperidin-4- yl)oxy)meth- yl)phenyl)carbamate |
| 233 | 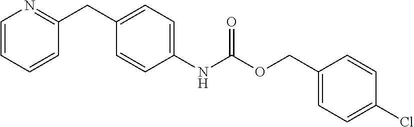 [SMILES: O=C(Nc1ccc(Cc2ccccn2)cc1)OCc1ccc(Cl)cc1] | 4-chlorobenzyl (4- (pyridin-2- ylmethyl)phenyl)carbamate |
| 234 | 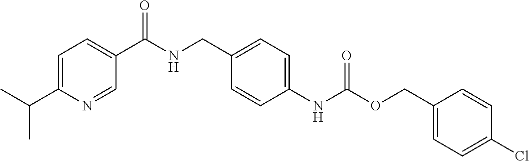 [SMILES: CC(C)c1ccc(C(=O)NCc2ccc(NC(=O)OCc3ccc(Cl)cc3)cc2)cn1] | 4-chlorobenzyl (4-((6- isopropylnicotinamido)meth- yl)phenyl)carbamate |
| 235 | 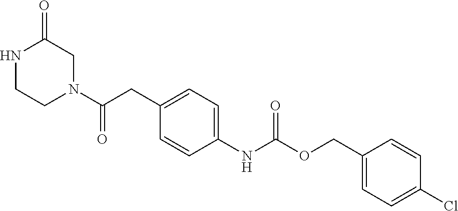 [SMILES: O=C1CN(C(=O)Cc2ccc(NC(=O)OCc3ccc(Cl)cc3)cc2)CCN1] | 4-chlorobenzyl (4-(2- oxo-2-(3-oxopiperazin-1- yl)ethyl)phenyl)carbamate |
| 236 | 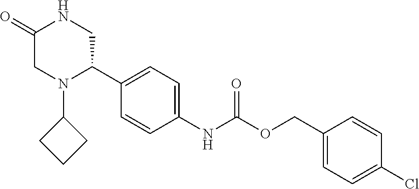 [SMILES: O=C1CN(C2CCC2)[C@@H](c2ccc(NC(=O)OCc3ccc(Cl)cc3)cc2)CN1] | 4-chlorobenzyl (S)-(4-(1- cyclobutyl-5- oxopiperazin-2- yl)phenyl)carbamate |
| 237 | 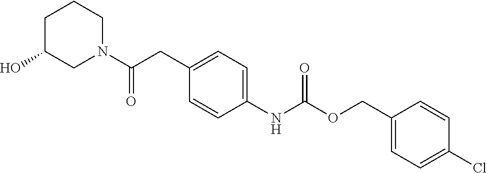 [SMILES: O=C(Nc1ccc(CC(=O)N2CCC[C@@H](O)C2)cc1)OCc1ccc(Cl)cc1] | 4-chlorobenzyl (R)-(4-(2- (3-hydroxypiperidin-1- yl)-2- oxoethyl)phenyl)carbamate |
| 238 | 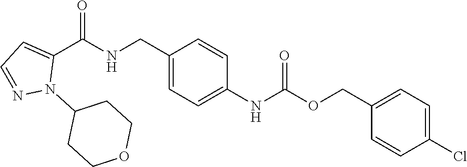 [SMILES: O=C(Nc1ccc(CNC(=O)c2ccnn2C2CCOCC2)cc1)OCc1ccc(Cl)cc1] | 4-chlorobenzyl (4-((1- (tetrahydro-2H-pyran-4- yl)-1H-pyrazole-5- carboxamido)meth- yl)phenyl)carbamate |
| 239 | 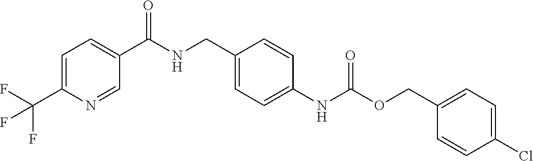 [SMILES: O=C(Nc1ccc(CNC(=O)c2ccc(C(F)(F)F)nc2)cc1)OCc1ccc(Cl)cc1] | 4-chlorobenzyl (4-((6- (trifluoromethyl)nicotin- amido)methyl)phenyl)carbamate |
| 240 | 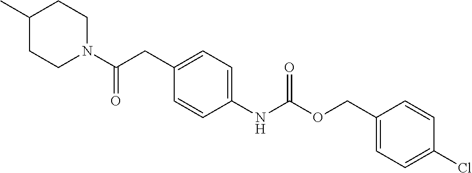 [SMILES: CC1CCN(C(=O)Cc2ccc(NC(=O)OCc3ccc(Cl)cc3)cc2)CC1] | 4-chlorobenzyl (4-(2-(4- methylpiperidin-1-yl)-2- oxoethyl)phenyl)carbamate |
| 241 | 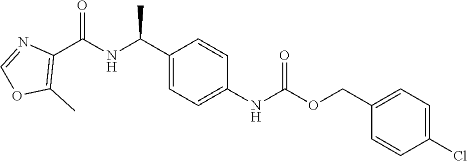 [SMILES: Cc1ocnc1C(=O)N[C@@H](C)c1ccc(NC(=O)OCc2ccc(Cl)cc2)cc1] | 4-chlorobenzyl (S)-(4-(1- (5-methyloxazole-4- carboxamido)eth- yl)phenyl)carbamate |
| 242 | 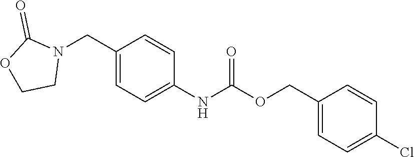 [SMILES: O=C(Nc1ccc(CN2CCOC2=O)cc1)OCc1ccc(Cl)cc1] | 4-chlorobenzyl (4-((2- oxooxazolidin-3- yl)methyl)phenyl)carbamate |
| 243 | 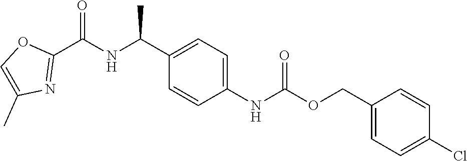 [SMILES: Cc1coc(C(=O)N[C@@H](C)c2ccc(NC(=O)OCc3ccc(Cl)cc3)cc2)n1] | 4-chlorobenzyl (S)-(4-(1- (4-methyloxazole-2- carboxamido)eth- yl)phenyl)carbamate |
| 244 | 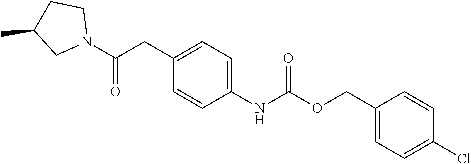 [SMILES: C[C@H]1CCN(C(=O)Cc2ccc(NC(=O)OCc3ccc(Cl)cc3)cc2)C1] | 4-chlorobenzyl (S)-(4-(2- (3-methylpyrrolidin-1-yl)-2- oxoethyl)phenyl)carbamate |
| 245 | 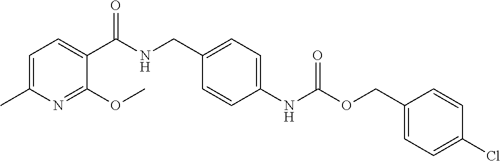 [SMILES: COc1nc(C)ccc1C(=O)NCc1ccc(NC(=O)OCc2ccc(Cl)cc2)cc1] | 4-chlorobenzyl (4-((2- methoxy-6- methylnicotinamido)meth- yl)phenyl)carbamate |
| 246 | 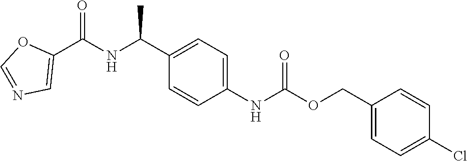 [SMILES: C[C@H](NC(=O)c1cnco1)c1ccc(NC(=O)OCc2ccc(Cl)cc2)cc1] | 4-chlorobenzyl (S)-(4-(1- (oxazole-5- carboxamido)eth- yl)phenyl)carbamate |
| 247 | 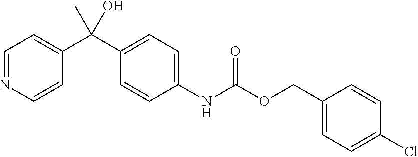 [SMILES: CC(O)(c1ccncc1)c1ccc(NC(=O)OCc2ccc(Cl)cc2)cc1] | 4-chlorobenzyl (4-(1- hydroxy-1-(pyridin-4- yl)ethyl)phenyl)carbamate |
| 248 | 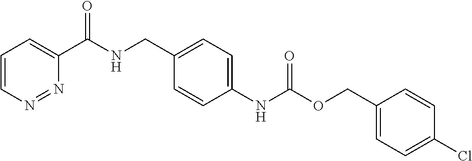 [SMILES: O=C(Nc1ccc(CNC(=O)c2cccnn2)cc1)OCc1ccc(Cl)cc1] | 4-chlorobenzyl (4- ((pyridazine-3- carboxamido)meth- yl)phenyl)carbamate |
| 249 | 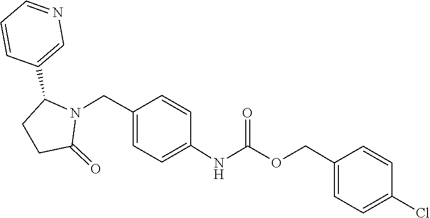 [SMILES: O=C(Nc1ccc(CN2C(=O)CC[C@@H]2c2cccnc2)cc1)OCc1ccc(Cl)cc1] | 4-chlorobenzyl (R)-(4- ((2-oxo-5-(pyridin-3- yl)pyrrolidin-1- yl)methyl)phenyl)carbamate |
| 250 | 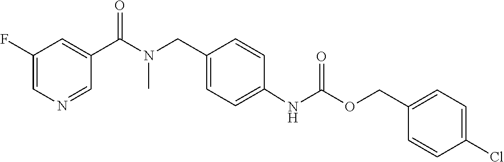 [SMILES: CN(Cc1ccc(NC(=O)OCc2ccc(Cl)cc2)cc1)C(=O)c1cncc(F)c1] | 4-chlorobenzyl (4-((5- fluoro-N- methylnicotinamido)meth- yl)phenyl)carbamate |
| 251 | 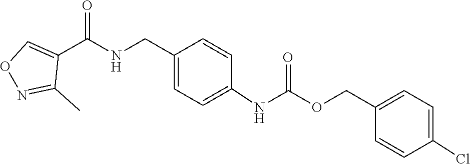 [SMILES: Cc1nocc1C(=O)NCc1ccc(NC(=O)OCc2ccc(Cl)cc2)cc1] | 4-chlorobenzyl (4-((3- methylisoxazole-4- carboxamido)meth- yl)phenyl)carbamate |
| 252 | 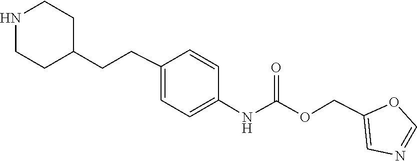 [SMILES: O=C(Nc1ccc(CCC2CCNCC2)cc1)OCc1cnco1] | oxazol-5-ylmethyl (4-(2- (piperidin-4- yl)ethyl)phenyl)carbamate |
| 253 | 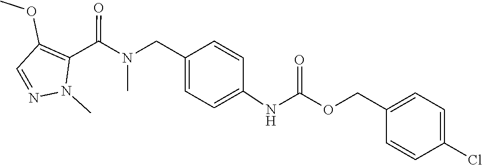 [SMILES: COc1cnn(C)c1C(=O)N(C)Cc1ccc(NC(=O)OCc2ccc(Cl)cc2)cc1] | 4-chlorobenzyl (4-((4- methoxy-N,1-dimethyl- 1H-pyrazole-5- carboxamido)meth- yl)phenyl)carbamate |
| 254 | 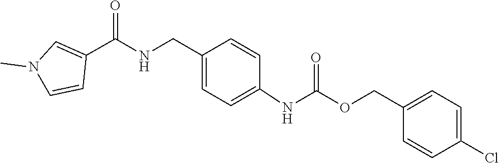 [SMILES: Cn1ccc(C(=O)NCc2ccc(NC(=O)OCc3ccc(Cl)cc3)cc2)c1] | 4-chlorobenzyl (4-((1- methyl-1H-pyrrole-3- carboxamido)meth- yl)phenyl)carbamate |
| 255 | 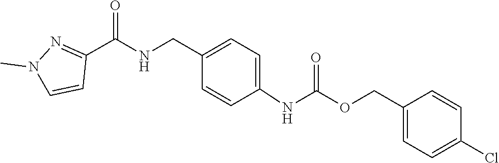 [SMILES: Cn1ccc(C(=O)NCc2ccc(NC(=O)OCc3ccc(Cl)cc3)cc2)n1] | 4-chlorobenzyl (4-((1- methyl-1H-pyrazole-3- carboxamido)meth- yl)phenyl)carbamate |
| 256 | 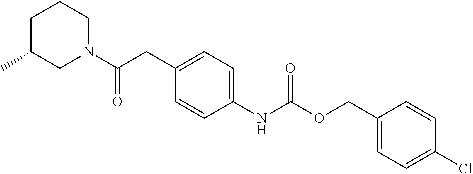 [SMILES: C[C@@H]1CCCN(C(=O)Cc2ccc(NC(=O)OCc3ccc(Cl)cc3)cc2)C1] | 4-chlorobenzyl (R)-(4-(2- (3-methylpiperidin-1-yl)-2- oxoethyl)phenyl)carbamate |
| 257 | 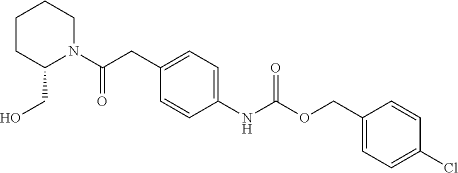 [SMILES: O=C(Nc1ccc(CC(=O)N2CCCC[C@H]2CO)cc1)OCc1ccc(Cl)cc1] | 4-chlorobenzyl (S)-(4-(2-(2- (hydroxymethyl)piperidin- 1-yl)-2- oxoethyl)phenyl)carbamate |
| 258 | 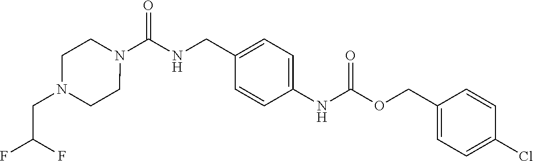 [SMILES: O=C(Nc1ccc(CNC(=O)N2CCN(CC(F)F)CC2)cc1)OCc1ccc(Cl)cc1] | 4-chlorobenzyl (4-((4-(2,2- difluoroethyl)piperazine-1- carboxamido)meth- yl)phenyl)carbamate |
| 259 | 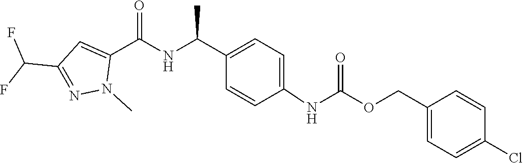 [SMILES: C[C@H](NC(=O)c1cc(C(F)F)nn1C)c1ccc(NC(=O)OCc2ccc(Cl)cc2)cc1] | 4-chlorobenzyl (S)-(4-(1- (3-(difluoromethyl)-1- methyl-1H-pyrazole-5- carboxamido)eth- yl)phenyl)carbamate |
| 260 | 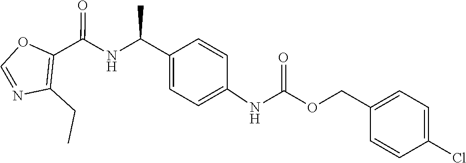 [SMILES: CCc1ncoc1C(=O)N[C@@H](C)c1ccc(NC(=O)OCc2ccc(Cl)cc2)cc1] | 4-chlorobenzyl (S)-(4-(1- (4-ethyloxazole-5- carboxamido)eth- yl)phenyl)carbamate |
| 261 | 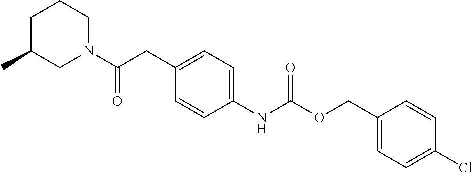 [SMILES: C[C@H]1CCCN(C(=O)Cc2ccc(NC(=O)OCc3ccc(Cl)cc3)cc2)C1] | 4-chlorobenzyl (S)-(4-(2- (3-methylpiperidin-1-yl)-2- oxoethyl)phenyl)carbamate |
| 262 | 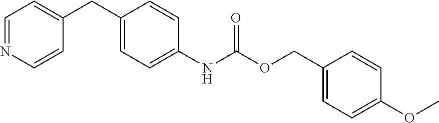 [SMILES: COc1ccc(COC(=O)Nc2ccc(Cc3ccncc3)cc2)cc1] | 4-methoxybenzyl (4- (pyridin-4- ylmethyl)phenyl)carbamate |
| 263 | 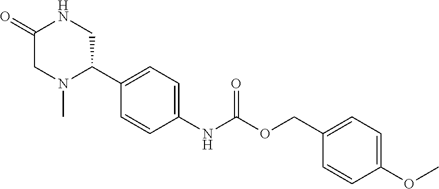 [SMILES: COc1ccc(COC(=O)Nc2ccc([C@H]3CNC(=O)CN3C)cc2)cc1] | 4-methoxybenzyl (S)-(4- (1-methyl-5- oxopiperazin-2- yl)phenyl)carbamate |
| 264 | 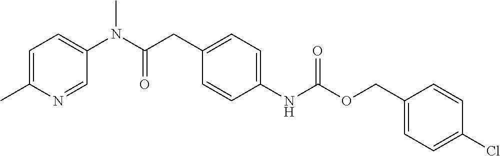 [SMILES: Cc1ccc(N(C)C(=O)Cc2ccc(NC(=O)OCc3ccc(Cl)cc3)cc2)cn1] | 4-chlorobenzyl (4-(2- (methyl(6-methylpyridin- 3-yl)amino)-2- oxoethyl)phenyl)carbamate |
| 265 | 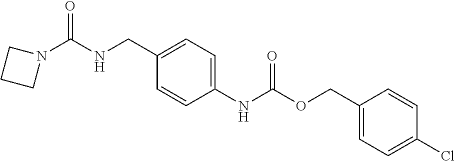 [SMILES: O=C(Nc1ccc(CNC(=O)N2CCC2)cc1)OCc1ccc(Cl)cc1] | 4-chlorobenzyl (4- ((azetidine-1- carboxamido)meth- yl)phenyl)carbamate |
| 266 | 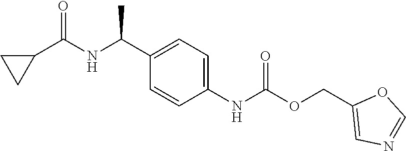 [SMILES: C[C@H](NC(=O)C1CC1)c1ccc(NC(=O)OCc2cnco2)cc1] | oxazol-5-ylmethyl (S)- (4-(1- (cyclopropanecarbox- amido)ethyl)phenyl)carbamate |
| 267 | 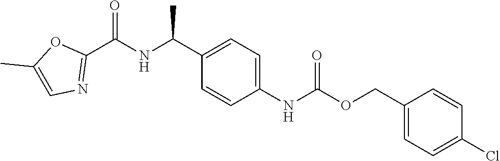 [SMILES: Cc1cnc(C(=O)N[C@@H](C)c2ccc(NC(=O)OCc3ccc(Cl)cc3)cc2)o1] | 4-chlorobenzyl (S)-(4-(1- (5-methyloxazole-2- carboxamido)eth- yl)phenyl)carbamate |
| 268 | 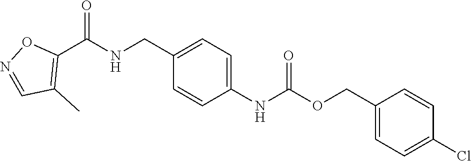 [SMILES: Cc1cnoc1C(=O)NCc1ccc(NC(=O)OCc2ccc(Cl)cc2)cc1] | 4-chlorobenzyl (4-((4- methylisoxazole-5- carboxamido)meth- yl)phenyl)carbamate |
| 269 | 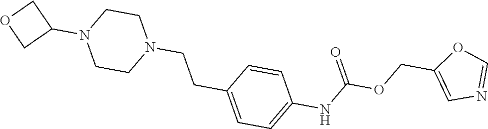 [SMILES: O=C(Nc1ccc(CCN2CCN(C3COC3)CC2)cc1)OCc1cnco1] | oxazol-5-ylmethyl (4-(2- (4-(oxetan-3- yl)piperazin-1- yl)ethyl)phenyl)carbamate |
| 270 | 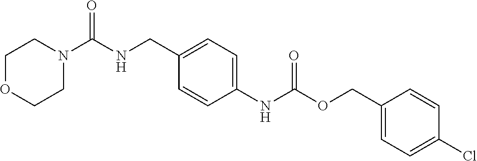 [SMILES: O=C(Nc1ccc(CNC(=O)N2CCOCC2)cc1)OCc1ccc(Cl)cc1] | 4-chlorobenzyl (4- ((morpholine-4- carboxamido)meth- yl)phenyl)carbamate |
| 271 | 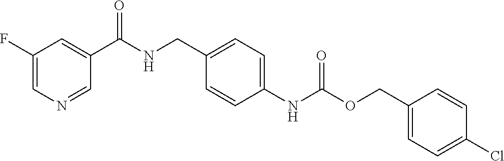 [SMILES: O=C(Nc1ccc(CNC(=O)c2cncc(F)c2)cc1)OCc1ccc(Cl)cc1] | 4-chlorobenzyl (4-((5- fluoronicotinamido)meth- yl)phenyl)carbamate |
| 272 | 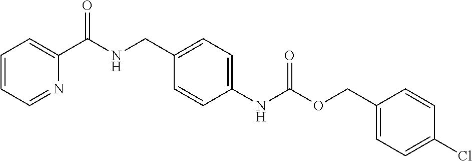 [SMILES: O=C(Nc1ccc(CNC(=O)c2ccccn2)cc1)OCc1ccc(Cl)cc1] | 4-chlorobenzyl (4- (picolinamidometh- yl)phenyl)carbamate |
| 273 | 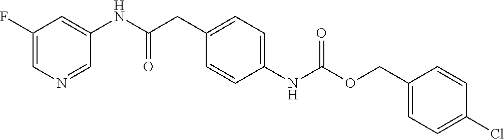 [SMILES: O=C(Cc1ccc(NC(=O)OCc2ccc(Cl)cc2)cc1)Nc1cncc(F)c1] | 4-chlorobenzyl (4-(2-((5- fluoropyridin-3- yl)amino)-2- oxoethyl)phenyl)carbamate |
| 274 | 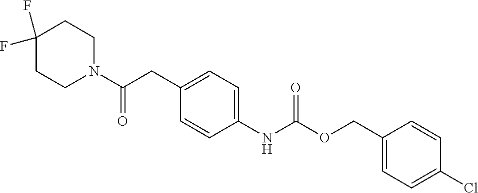 [SMILES: O=C(Nc1ccc(CC(=O)N2CCC(F)(F)CC2)cc1)OCc1ccc(Cl)cc1] | 4-chlorobenzyl (4-(2- (4,4-difluoropiperidin-1- yl)-2- oxoethyl)phenyl)carbamate |
| 275 | 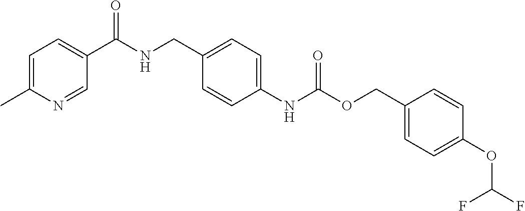 [SMILES: Cc1ccc(C(=O)NCc2ccc(NC(=O)OCc3ccc(OC(F)F)cc3)cc2)cn1] | 4- (difluoromethoxy)benzyl (4-((6- methylnicotinamido)meth- yl)phenyl)carbamate |
| 276 | 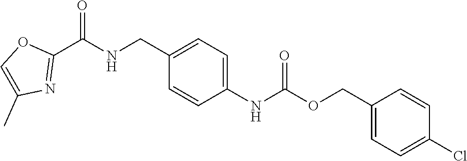 [SMILES: Cc1coc(C(=O)NCc2ccc(NC(=O)OCc3ccc(Cl)cc3)cc2)n1] | 4-chlorobenzyl (4-((4- methyloxazole-2- carboxamido)meth- yl)phenyl)carbamate |
| 277 | 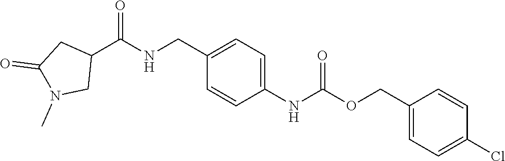 [SMILES: CN1CC(C(=O)NCc2ccc(NC(=O)OCc3ccc(Cl)cc3)cc2)CC1=O] | 4-chlorobenzyl (4-((1- methyl-5-oxopyrrolidine-3- carboxamido)meth- yl)phenyl)carbamate |
| 278 | 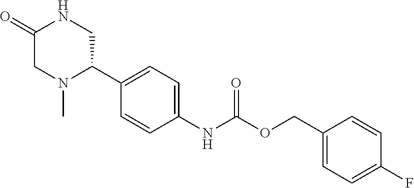 [SMILES: CN1CC(=O)NC[C@@H]1c1ccc(NC(=O)OCc2ccc(F)cc2)cc1] | 4-fluorobenzyl (S)-(4-(1- methyl-5-oxopiperazin-2- yl)phenyl)carbamate |
| 279 | 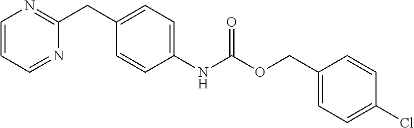 [SMILES: O=C(Nc1ccc(Cc2ncccn2)cc1)OCc1ccc(Cl)cc1] | 4-chlorobenzyl (4- (pyrimidin-2- ylmethyl)phenyl)carbamate |
| 280 | 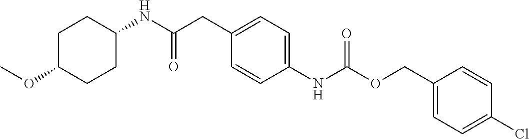 [SMILES: CO[C@H]1CC[C@@H](NC(=O)Cc2ccc(NC(=O)OCc3ccc(Cl)cc3)cc2)CC1] | 4-chlorobenzyl (4-(2- (((1s,4s)-4- methoxycyclohexyl)amino)-2- oxoethyl)phenyl)carbamate |
| 281 | 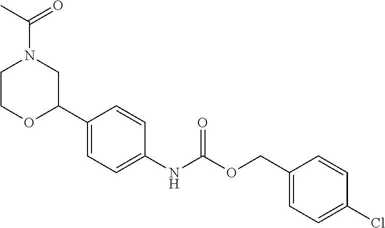 [SMILES: CC(=O)N1CCOC(c2ccc(NC(=O)OCc3ccc(Cl)cc3)cc2)C1] | 4-chlorobenzyl (4-(4- acetylmorpholin-2- yl)phenyl)carbamate |
| 282 | 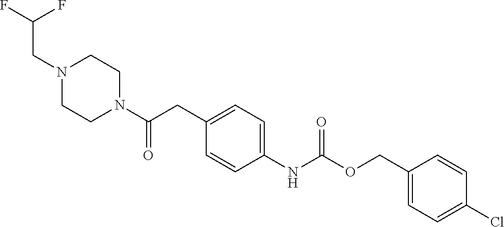 [SMILES: O=C(Nc1ccc(CC(=O)N2CCN(CC(F)F)CC2)cc1)OCc1ccc(Cl)cc1] | 4-chlorobenzyl (4-(2-(4- (2,2- difluoroethyl)piperazin- 1-yl)-2- oxoethyl)phenyl)carbamate |
| 283 | 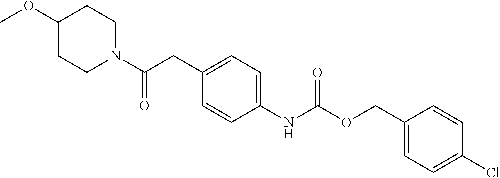 [SMILES: COC1CCN(C(=O)Cc2ccc(NC(=O)OCc3ccc(Cl)cc3)cc2)CC1] | 4-chlorobenzyl (4-(2-(4- methoxypiperidin-1-yl)-2- oxoethyl)phenyl)carbamate |
| 284 | 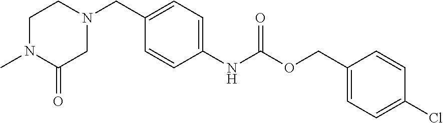 [SMILES: CN1CCN(Cc2ccc(NC(=O)OCc3ccc(Cl)cc3)cc2)CC1=O] | 4-chlorobenzyl (4-((4- methyl-3-oxopiperazin-1- yl)methyl)phenyl)carbamate |
| 285 | 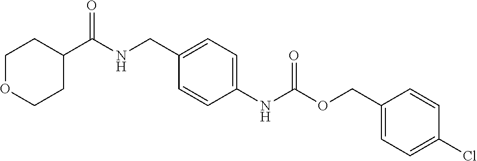 [SMILES: O=C(Nc1ccc(CNC(=O)C2CCOCC2)cc1)OCc1ccc(Cl)cc1] | 4-chlorobenzyl (4- ((tetrahydro-2H-pyran-4- carboxamido)meth- yl)phenyl)carbamate |
| 286 | 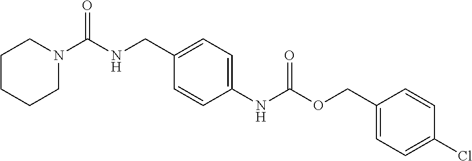 [SMILES: O=C(Nc1ccc(CNC(=O)N2CCCCC2)cc1)OCc1ccc(Cl)cc1] | 4-chlorobenzyl (4- ((piperidine-1- carboxamido)meth- yl)phenyl)carbamate |
| 287 | 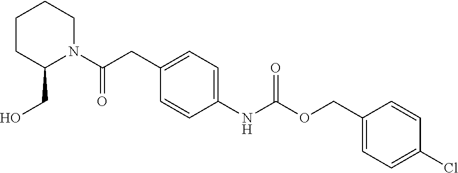 [SMILES: O=C(Nc1ccc(CC(=O)N2CCCC[C@@H]2CO)cc1)OCc1ccc(Cl)cc1] | 4-chlorobenzyl (R)-(4-(2-(2- (hydroxymethyl)piperidin- 1-yl)-2- oxoethyl)phenyl)carbamate |
| 288 | 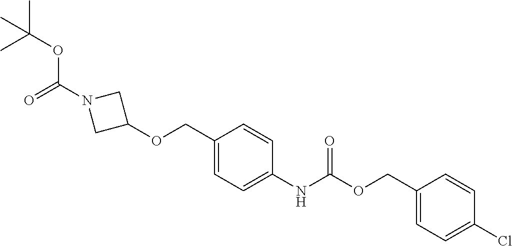 [SMILES: CC(C)(C)OC(=O)N1CC(OCc2ccc(NC(=O)OCc3ccc(Cl)cc3)cc2)C1] | tert-butyl 3-((4-((((4- chlorobenzyl)oxy)car- bonyl)amino)benzyl)oxy)aze- tidine-1-carboxylate |
| 289 | 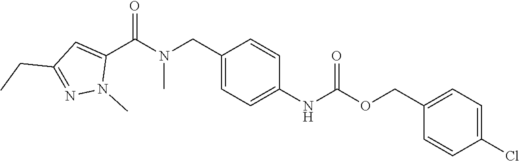 [SMILES: CCc1cc(C(=O)N(C)Cc2ccc(NC(=O)OCc3ccc(Cl)cc3)cc2)n(C)n1] | 4-chlorobenzyl (4-((3- ethyl-N,1-dimethyl-1H- pyrazole-5- carboxamido)meth- yl)phenyl)carbamate |
| 290 | 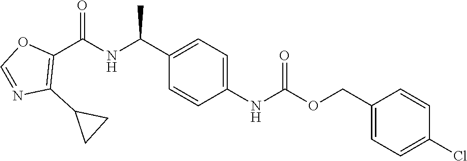 [SMILES: C[C@H](NC(=O)c1ocnc1C1CC1)c1ccc(NC(=O)OCc2ccc(Cl)cc2)cc1] | 4-chlorobenzyl (S)-(4-(1- (4-cyclopropyloxazole-5- carboxamido)eth- yl)phenyl)carbamate |
| 291 | 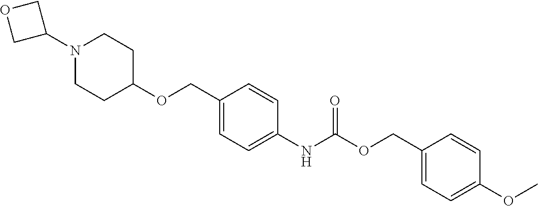 [SMILES: COc1ccc(COC(=O)Nc2ccc(COC3CCN(C4COC4)CC3)cc2)cc1] | 4-methoxybenzyl (4-(((1- (oxetan-3-yl)piperidin-4- yl)oxy)methyl)phenyl)carbamate |
| 292 | 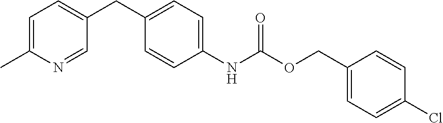 [SMILES: Cc1ccc(Cc2ccc(NC(=O)OCc3ccc(Cl)cc3)cc2)cn1] | 4-chlorobenzyl (4-((6- methylpyridin-3- yl)methyl)phenyl)carbamate |
| 293 | 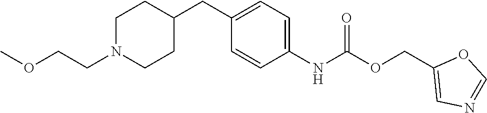 [SMILES: COCCN1CCC(Cc2ccc(NC(=O)OCc3cnco3)cc2)CC1] | oxazol-5-ylmethyl (4-((1-(2- methoxyethyl)piperidin-4- yl)methyl)phenyl)carbamate |
| 294 | 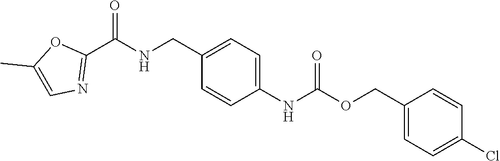 [SMILES: Cc1cnc(C(=O)NCc2ccc(NC(=O)OCc3ccc(Cl)cc3)cc2)o1] | 4-chlorobenzyl (4-((5- methyloxazole-2- carboxamido)meth- yl)phenyl)carbamate |
| 295 | 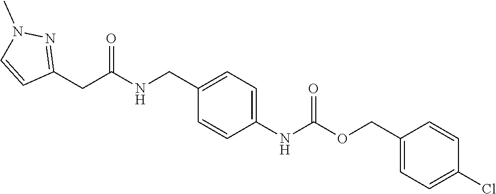 [SMILES: Cn1ccc(CC(=O)NCc2ccc(NC(=O)OCc3ccc(Cl)cc3)cc2)n1] | 4-chlorobenzyl (4-((2-(1- methyl-1H-pyrazol-3- yl)acetamido)meth- yl)phenyl)carbamate |
| 296 | 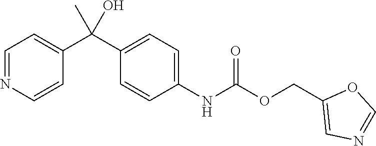 [SMILES: CC(O)(c1ccncc1)c1ccc(NC(=O)OCc2cnco2)cc1] | oxazol-5-ylmethyl (4-(1- hydroxy-1-(pyridin-4- yl)ethyl)phenyl)carbamate |
| 297 | 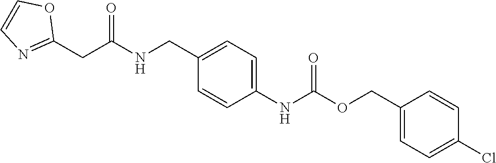 [SMILES: O=C(Cc1ncco1)NCc1ccc(NC(=O)OCc2ccc(Cl)cc2)cc1] | 4-chlorobenzyl (4-((2- (oxazol-2- yl)acetamido)meth- yl)phenyl)carbamate |
| 298 | 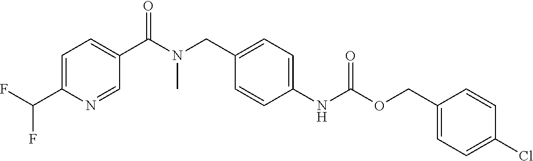 [SMILES: CN(Cc1ccc(NC(=O)OCc2ccc(Cl)cc2)cc1)C(=O)c1ccc(C(F)F)nc1] | 4-chlorobenzyl (4-((6- (difluoromethyl)-N- methylnicotinamido)meth- yl)phenyl)carbamate |
| 299 | 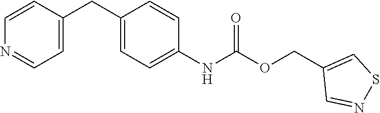 [SMILES: O=C(Nc1ccc(Cc2ccncc2)cc1)OCc1cnsc1] | isothiazol-4-ylmethyl (4- (pyridin-4- ylmethyl)phenyl)carbamate |
| 300 | 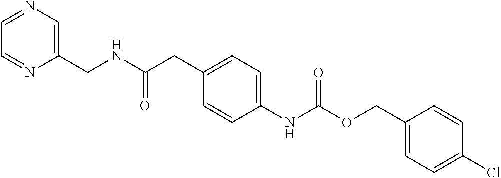 [SMILES: O=C(Cc1ccc(NC(=O)OCc2ccc(Cl)cc2)cc1)NCc1cnccn1] | 4-chlorobenzyl (4-(2- oxo-2-((pyrazin-2- ylmethyl)amino)eth- yl)phenyl)carbamate |
| 301 | 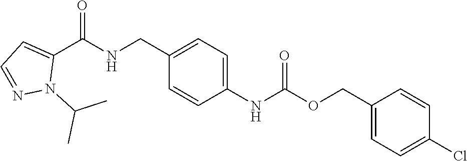 [SMILES: CC(C)n1nccc1C(=O)NCc1ccc(NC(=O)OCc2ccc(Cl)cc2)cc1] | 4-chlorobenzyl (4-((1- isopropyl-1H-pyrazole-5- carboxamido)meth- yl)phenyl)carbamate |
| 302 | 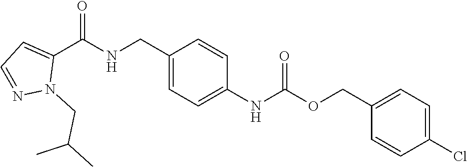 [SMILES: CC(C)Cn1nccc1C(=O)NCc1ccc(NC(=O)OCc2ccc(Cl)cc2)cc1] | 4-chlorobenzyl (4-((1- isobutyl-1H-pyrazole-5- carboxamido)meth- yl)phenyl)carbamate |
| 303 | 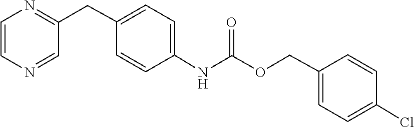 [SMILES: O=C(Nc1ccc(Cc2cnccn2)cc1)OCc1ccc(Cl)cc1] | 4-chlorobenzyl (4- (pyrazin-2- ylmethyl)phenyl)carbamate |
| 304 | 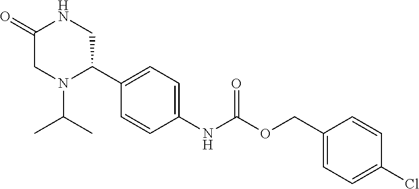 [SMILES: CC(C)N1CC(=O)NC[C@@H]1c1ccc(NC(=O)OCc2ccc(Cl)cc2)cc1] | 4-chlorobenzyl (S)-(4-(1- isopropyl-5- oxopiperazin-2- yl)phenyl)carbamate |
| 305 | 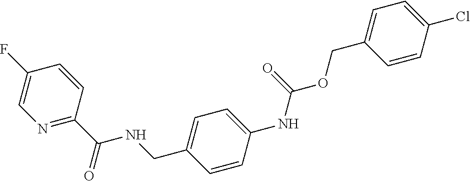 [SMILES: O=C(Nc1ccc(CNC(=O)c2ccc(F)cn2)cc1)OCc1ccc(Cl)cc1] | 4-chlorobenzyl (4-((5- fluoropicolinamido)meth- yl)phenyl)carbamate |
| 306 | 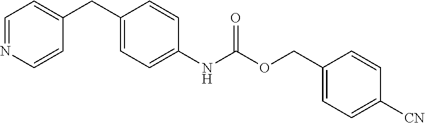 [SMILES: N#Cc1ccc(COC(=O)Nc2ccc(Cc3ccncc3)cc2)cc1] | 4-cyanobenzyl (4- (pyridin-4- ylmethyl)phenyl)carbamate |
| 307 | 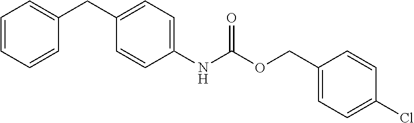 [SMILES: O=C(Nc1ccc(Cc2ccccc2)cc1)OCc1ccc(Cl)cc1] | 4-chlorobenzyl (4- benzylphenyl)carbamate |
| 308 | 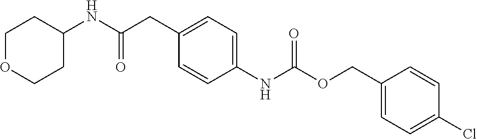 [SMILES: O=C(Cc1ccc(NC(=O)OCc2ccc(Cl)cc2)cc1)NC1CCOCC1] | 4-chlorobenzyl (4-(2- oxo-2-((tetrahydro-2H- pyran-4- yl)amino)ethyl)phenyl)carbamate |
| 309 | 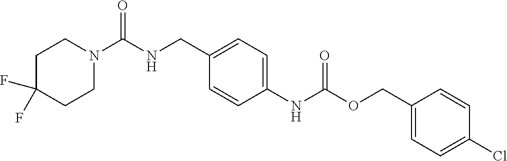 [SMILES: O=C(Nc1ccc(CNC(=O)N2CCC(F)(F)CC2)cc1)OCc1ccc(Cl)cc1] | 4-chlorobenzyl (4-((4,4- difluoropiperidine-1- carboxamido)meth- yl)phenyl)carbamate |
| 310 | 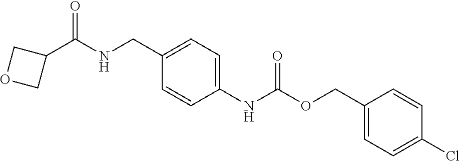 [SMILES: O=C(Nc1ccc(CNC(=O)C2COC2)cc1)OCc1ccc(Cl)cc1] | 4-chlorobenzyl (4- ((oxetane-3- carboxamido)meth- yl)phenyl)carbamate |
| 311 | 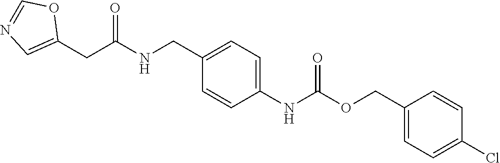 [SMILES: O=C(Cc1cnco1)NCc1ccc(NC(=O)OCc2ccc(Cl)cc2)cc1] | 4-chlorobenzyl (4-((2- (oxazol-5- yl)acetamido)meth- yl)phenyl)carbamate |
| 312 | 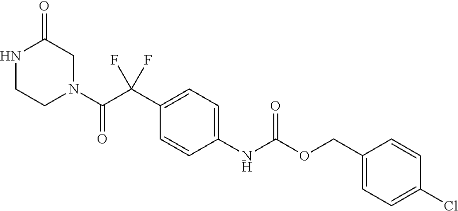 [SMILES: O=C1CN(C(=O)C(F)(F)c2ccc(NC(=O)OCc3ccc(Cl)cc3)cc2)CCN1] | 4-chlorobenzyl (4-(1,1- difluoro-2-oxo-2-(3- oxopiperazin-1- yl)ethyl)phenyl)carbamate |
| 313 | 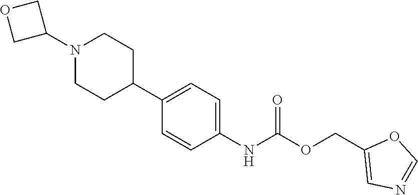 [SMILES: O=C(Nc1ccc(C2CCN(C3COC3)CC2)cc1)OCc1cnco1] | oxazol-5-ylmethyl (4-(1- (oxetan-3-yl)piperidin-4- yl)phenyl)carbamate |
| 314 | 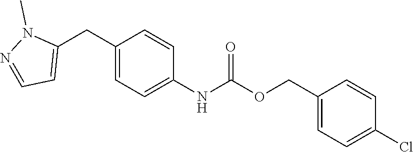 [SMILES: Cn1nccc1Cc1ccc(NC(=O)OCc2ccc(Cl)cc2)cc1] | 4-chlorobenzyl (4-((1- methyl-1H-pyrazol-5- yl)methyl)phenyl)carbamate |
| 315 | 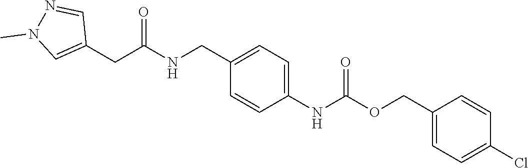 [SMILES: Cn1cc(CC(=O)NCc2ccc(NC(=O)OCc3ccc(Cl)cc3)cc2)cn1] | 4-chlorobenzyl (4-((2-(1- methyl-1H-pyrazol-4- yl)acetamido)meth- yl)phenyl)carbamate |
| 316 | 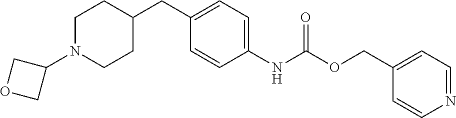 [SMILES: O=C(Nc1ccc(CC2CCN(C3COC3)CC2)cc1)OCc1ccncc1] | pyridin-4-ylmethyl (4- ((1-(oxetan-3- yl)piperidin-4- yl)methyl)phenyl)carbamate |
| 317 | 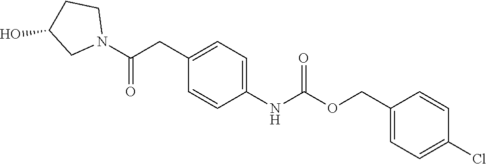 [SMILES: O=C(Nc1ccc(CC(=O)N2CC[C@@H](O)C2)cc1)OCc1ccc(Cl)cc1] | 4-chlorobenzyl (R)-(4-(2- (3-hydroxypyrrolidin-1- yl)-2- oxoethyl)phenyl)carbamate |
| 318 | 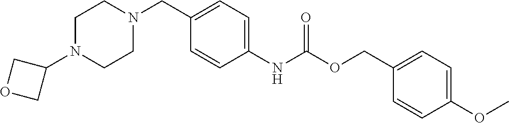 [SMILES: COc1ccc(COC(=O)Nc2ccc(CN3CCN(C4COC4)CC3)cc2)cc1] | 4-methoxybenzyl (4-((4- (oxetan-3-yl)piperazin-1- yl)methyl)phenyl)carbamate |
| 319 | 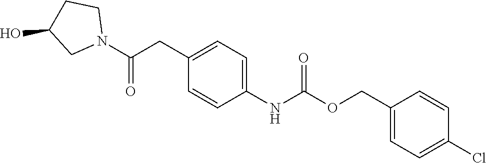 [SMILES: O=C(Nc1ccc(CC(=O)N2CC[C@H](O)C2)cc1)OCc1ccc(Cl)cc1] | 4-chlorobenzyl (S)-(4-(2- (3-hydroxypyrrolidin-1-yl)-2- oxoethyl)phenyl)carbamate |
| 320 | 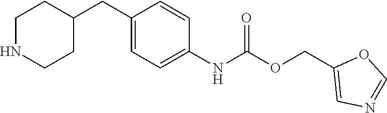 [SMILES: O=C(Nc1ccc(CC2CCNCC2)cc1)OCc1cnco1] | oxazol-5-ylmethyl (4- (piperidin-4- ylmethyl)phenyl)carbamate |
| 321 | 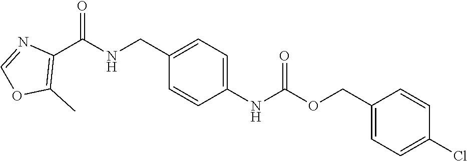 [SMILES: Cc1ocnc1C(=O)NCc1ccc(NC(=O)OCc2ccc(Cl)cc2)cc1] | 4-chlorobenzyl (4-((5- methyloxazole-4- carboxamido)meth- yl)phenyl)carbamate |
| 322 | 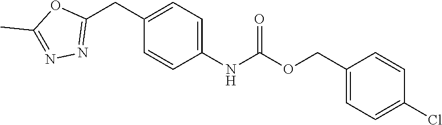 [SMILES: Cc1nnc(Cc2ccc(NC(=O)OCc3ccc(Cl)cc3)cc2)o1] | 4-chlorobenzyl (4-((5- methyl-1,3,4-oxadiazol-2- yl)methyl)phenyl)carbamate |
| 323 | 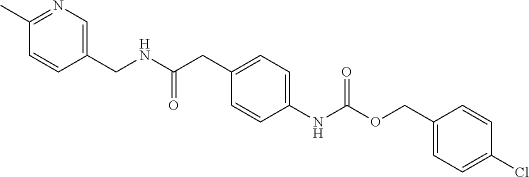 [SMILES: Cc1ccc(CNC(=O)Cc2ccc(NC(=O)OCc3ccc(Cl)cc3)cc2)cn1] | 4-chlorobenzyl (4-(2- (((6-methylpyridin-3- yl)methyl)amino)-2- oxoethyl)phenyl)carbamate |
| 324 | 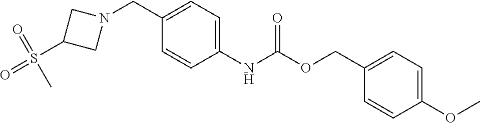 [SMILES: COc1ccc(COC(=O)Nc2ccc(CN3CC(S(C)(=O)=O)C3)cc2)cc1] | 4-methoxybenzyl (4-((3- (methylsulfonyl)azetidin-1- yl)methyl)phenyl)carbamate |
| 325 | 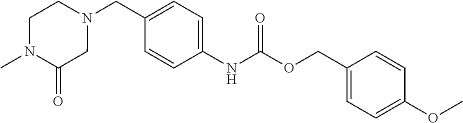 | 4-methoxybenzyl (4-((4- methyl-3-oxopiperazin-1- yl)methyl)phenyl)carbamate |
| 326 | 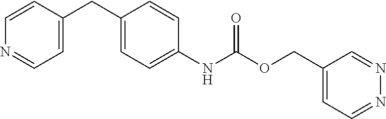 [SMILES: O=C(Nc1ccc(Cc2ccncc2)cc1)OCc1ccnnc1] | pyridazin-4-ylmethyl (4- (pyridin-4- ylmethyl)phenyl)carbamate |
| 327 | 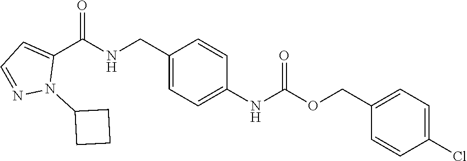 [SMILES: O=C(Nc1ccc(CNC(=O)c2ccnn2C2CCC2)cc1)OCc1ccc(Cl)cc1] | 4-chlorobenzyl (4-((1- cyclobutyl-1H-pyrazole-5- carboxamido)meth- yl)phenyl)carbamate |
| 328 | 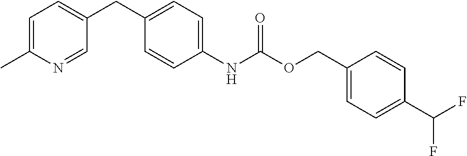 [SMILES: Cc1ccc(Cc2ccc(NC(=O)OCc3ccc(C(F)F)cc3)cc2)cn1] | 4-(difluoromethyl)benzyl (4-((6-methylpyridin-3- yl)methyl)phenyl)carbamate |
| 329 | 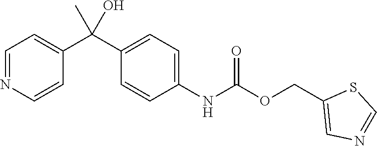 [SMILES: CC(O)(c1ccncc1)c1ccc(NC(=O)OCc2cncs2)cc1] | thiazol-5-ylmethyl (4-(1- hydroxy-1-(pyridin-4- yl)ethyl)phenyl)carbamate |
| 330 | 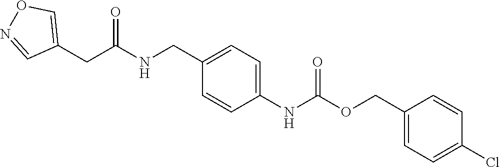 [SMILES: O=C(Cc1cnoc1)NCc1ccc(NC(=O)OCc2ccc(Cl)cc2)cc1] | 4-chlorobenzyl (4-((2- (isoxazol-4- yl)acetamido)meth- yl)phenyl)carbamate |
| 331 | 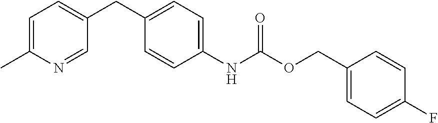 [SMILES: Cc1ccc(Cc2ccc(NC(=O)OCc3ccc(F)cc3)cc2)cn1] | 4-fluorobenzyl (4-((6- methylpyridin-3- yl)methyl)phenyl)carbamate |
| 332 | 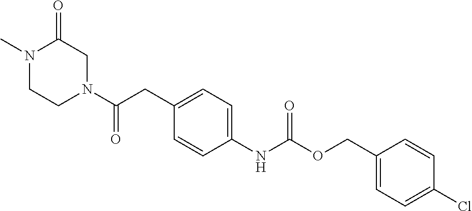 [SMILES: CN1CCN(C(=O)Cc2ccc(NC(=O)OCc3ccc(Cl)cc3)cc2)CC1=O] | 4-chlorobenzyl (4-(2-(4- methyl-3-oxopiperazin-1- yl)-2- oxoethyl)phenyl)carbamate |
| 333 | 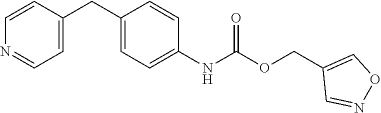 [SMILES: O=C(Nc1ccc(Cc2ccncc2)cc1)OCc1cnoc1] | isoxazol-4-ylmethyl (4- (pyridin-4- ylmethyl)phenyl)carbamate |
| 334 | 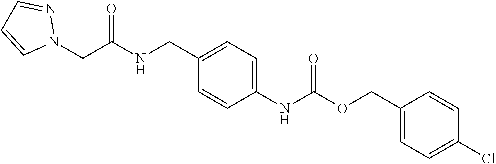 [SMILES: O=C(Cn1cccn1)NCc1ccc(NC(=O)OCc2ccc(Cl)cc2)cc1] | 4-chlorobenzyl (4-((2- (1H-pyrazol-1- yl)acetamido)meth- yl)phenyl)carbamate |
| 335 | 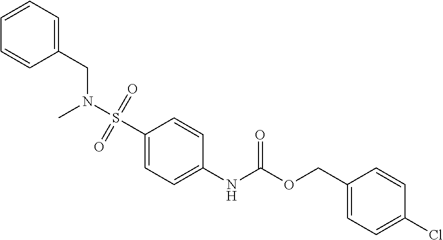 [SMILES: CN(Cc1ccccc1)S(=O)(=O)c1ccc(NC(=O)OCc2ccc(Cl)cc2)cc1] | 4-chlorobenzyl (4-(N- benzyl-N-methyl- sulfamoyl)phenyl)carbamate |
| 336 | 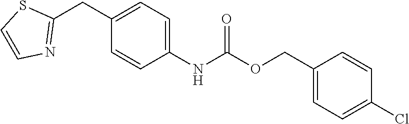 [SMILES: O=C(Nc1ccc(Cc2nccs2)cc1)OCc1ccc(Cl)cc1] | 4-chlorobenzyl (4- (thiazol-2- ylmethyl)phenyl)carbamate |
| 337 | 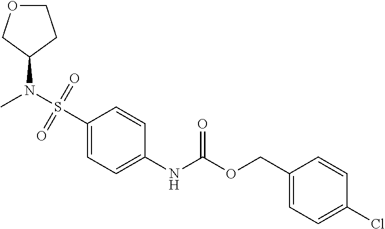 [SMILES: CN([C@@H]1CCOC1)S(=O)(=O)c1ccc(NC(=O)OCc2ccc(Cl)cc2)cc1] | 4-chlorobenzyl (R)-(4- (N-methyl-N- (tetrahydrofuran-3- yl)sulfamoyl)phenyl)carbamate |
| 338 | 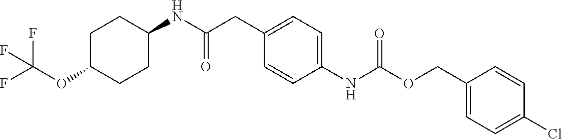 [SMILES: O=C(Cc1ccc(NC(=O)OCc2ccc(Cl)cc2)cc1)N[C@H]1CC[C@H](OC(F)(F)F)CC1] | 4-chlorobenzyl (4-(2- oxo-2-(((1r,4r)-4- (trifluoromethoxy)cyclo- hexyl)amino)eth- yl)phenyl)carbamate |
| 339 | 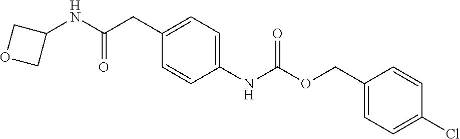 [SMILES: O=C(Cc1ccc(NC(=O)OCc2ccc(Cl)cc2)cc1)NC1COC1] | 4-chlorobenzyl (4-(2- (oxetan-3-ylamino)-2- oxoethyl)phenyl)carbamate |
| 340 | 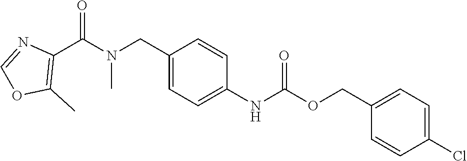 [SMILES: Cc1ocnc1C(=O)N(C)Cc1ccc(NC(=O)OCc2ccc(Cl)cc2)cc1] | 4-chlorobenzyl (4-((N,5- dimethyloxazole-4- carboxamido)meth- yl)phenyl)carbamate |
| 341 | 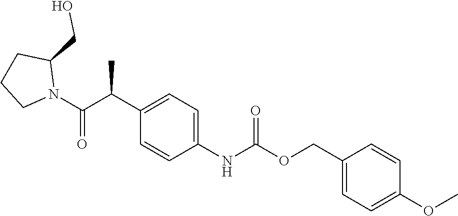 [SMILES: COc1ccc(COC(=O)Nc2ccc([C@H](C)C(=O)N3CCC[C@H]3CO)cc2)cc1] | 4-methoxybenzyl (4-((S)- 1-((S)-2- (hydroxymethyl)pyrrolidin- 1-yl)-1-oxopropan-2- yl)phenyl)carbamate |
| 342 | 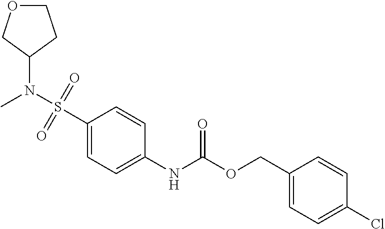 [SMILES: CN(C1CCOC1)S(=O)(=O)c1ccc(NC(=O)OCc2ccc(Cl)cc2)cc1] | 4-chlorobenzyl (4-(N- methyl-N- (tetrahydrofuran-3- yl)sulfamoyl)phenyl)carbamate |
| 343 | 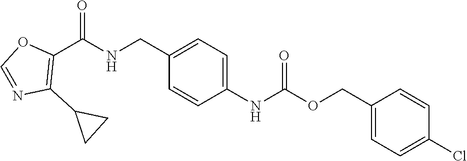 [SMILES: O=C(Nc1ccc(CNC(=O)c2ocnc2C2CC2)cc1)OCc1ccc(Cl)cc1] | 4-chlorobenzyl (4-((4- cyclopropyloxazole-5- carboxamido)meth- yl)phenyl)carbamate |
| 344 | 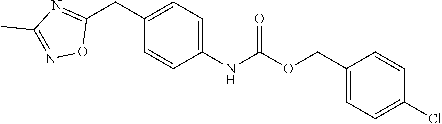 [SMILES: Cc1noc(Cc2ccc(NC(=O)OCc3ccc(Cl)cc3)cc2)n1] | 4-chlorobenzyl (4-((3- methyl-1,2,4-oxadiazol-5- yl)methyl)phenyl)carbamate |
| 345 | 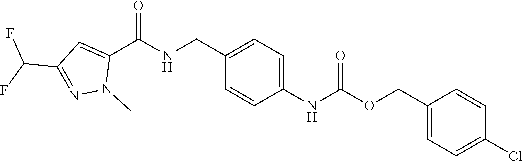 [SMILES: Cn1nc(C(F)F)cc1C(=O)NCc1ccc(NC(=O)OCc2ccc(Cl)cc2)cc1] | 4-chlorobenzyl (4-((3- (difluoromethyl)-1- methyl-1H-pyrazole-5- carboxamido)meth- yl)phenyl)carbamate |
| 346 | 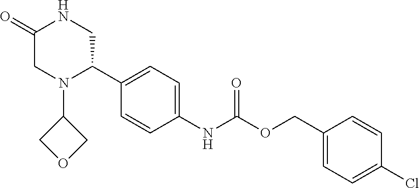 [SMILES: O=C1CN(C2COC2)[C@@H](c2ccc(NC(=O)OCc3ccc(Cl)cc3)cc2)CN1] | 4-chlorobenzyl (S)-(4-(1- (oxetan-3-yl)-5- oxopiperazin-2- yl)phenyl)carbamate |
| 347 | 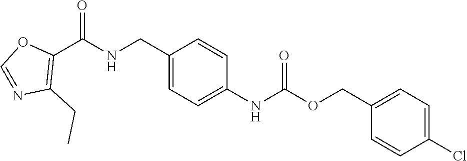 [SMILES: CCc1ncoc1C(=O)NCc1ccc(NC(=O)OCc2ccc(Cl)cc2)cc1] | 4-chlorobenzyl (4-((4- ethyloxazole-5- carboxamido)meth- yl)phenyl)carbamate |
| 348 | 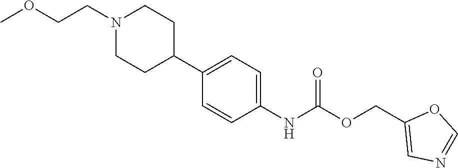 [SMILES: COCCN1CCC(c2ccc(NC(=O)OCc3cnco3)cc2)CC1] | oxazol-5-ylmethyl (4-(1-(2- methoxyethyl)piperidin- 4-yl)phenyl)carbamate |
| 349 | 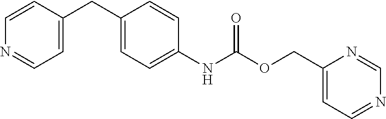 [SMILES: O=C(Nc1ccc(Cc2ccncc2)cc1)OCc1ccncn1] | pyrimidin-4-ylmethyl (4- (pyridin-4- ylmethyl)phenyl)carbamate |
| 350 | 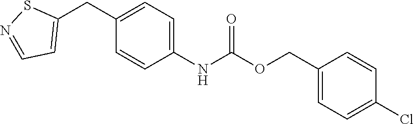 [SMILES: O=C(Nc1ccc(Cc2ccns2)cc1)OCc1ccc(Cl)cc1] | 4-chlorobenzyl (4- (isothiazol-5- ylmethyl)phenyl)carbamate |
| 351 | 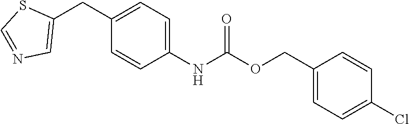 [SMILES: O=C(Nc1ccc(Cc2cncs2)cc1)OCc1ccc(Cl)cc1] | 4-chlorobenzyl (4- (thiazol-5- ylmethyl)phenyl)carbamate |
| 352 | 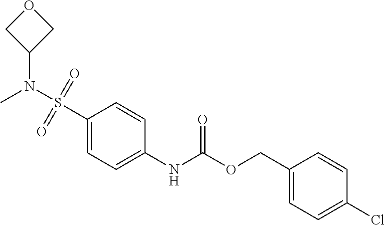 [SMILES: CN(C1COC1)S(=O)(=O)c1ccc(NC(=O)OCc2ccc(Cl)cc2)cc1] | 4-chlorobenzyl (4-(N- methyl-N-(oxetan-3- yl)sulfamoyl)phenyl)carbamate |
| 353 | 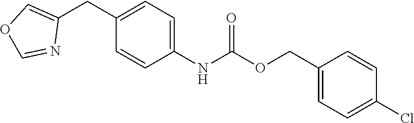 [SMILES: O=C(Nc1ccc(Cc2cocn2)cc1)OCc1ccc(Cl)cc1] | 4-chlorobenzyl (4- (oxazol-4- ylmethyl)phenyl)carbamate |
| 354 | 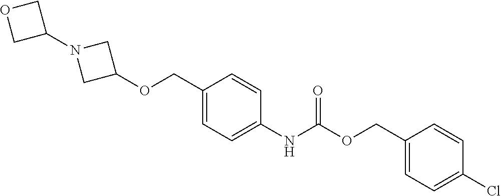 [SMILES: O=C(Nc1ccc(COC2CN(C3COC3)C2)cc1)OCc1ccc(Cl)cc1] | 4-chlorobenzyl (4-(((1- (oxetan-3-yl)azetidin-3- yl)oxy)methyl)phenyl)carbamate |
| 355 | 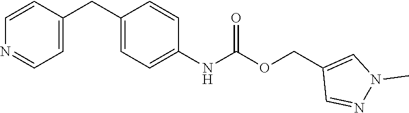 [SMILES: Cn1cc(COC(=O)Nc2ccc(Cc3ccncc3)cc2)cn1] | (1-methyl-1H-pyrazol-4- yl)methyl (4-(pyridin-4- ylmethyl)phenyl)carbamate |
| 356 | 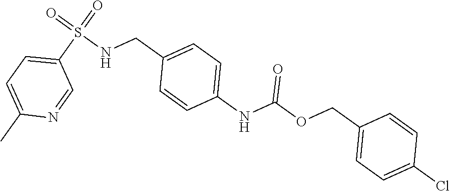 [SMILES: Cc1ccc(S(=O)(=O)NCc2ccc(NC(=O)OCc3ccc(Cl)cc3)cc2)cn1] | 4-chlorobenzyl (4-(((6- methylpyridine)-3- sulfonamido)meth- yl)phenyl)carbamate |
| 357 | 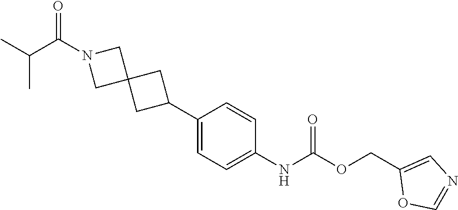 [SMILES: CC(C)C(=O)N1CC2(CC(c3ccc(NC(=O)OCc4cnco4)cc3)C2)C1] | oxazol-5-ylmethyl (4-(2- isobutyryl-2- azaspiro[3.3]heptan-6- yl)phenyl)carbamate |
| 358 | 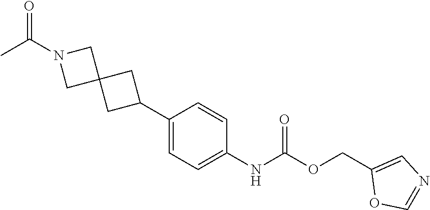 [SMILES: CC(=O)N1CC2(CC(c3ccc(NC(=O)OCc4cnco4)cc3)C2)C1] | oxazol-5-ylmethyl (4-(2- acetyl-2- azaspiro[3.3]heptan-6- yl)phenyl)carbamate |
| 359 | 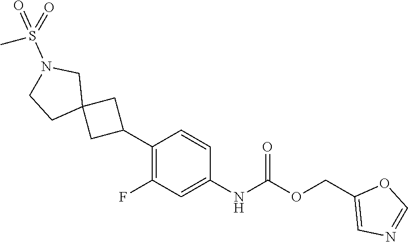 [SMILES: CS(=O)(=O)N1CCC2(CC(c3ccc(NC(=O)OCc4cnco4)cc3F)C2)C1] | |
| | Diastereoisomer 1 | |
| 360 | 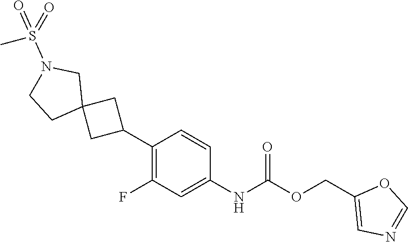 [SMILES: CS(=O)(=O)N1CCC2(CC(c3ccc(NC(=O)OCc4cnco4)cc3F)C2)C1] | |
| | Diastereoisomer 2 | |
| 361 | 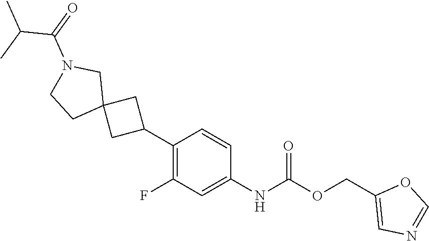 [SMILES: CC(C)C(=O)N1CCC2(CC(c3ccc(NC(=O)OCc4cnco4)cc3F)C2)C1] | |
| | Diastereoisomer 1 | |
| 362 | 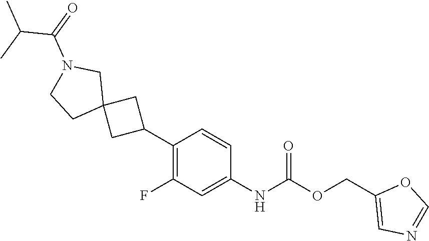 [SMILES: CC(C)C(=O)N1CCC2(CC(c3ccc(NC(=O)OCc4cnco4)cc3F)C2)C1] | |
| | Diastereoisomer 2 | |
| 363 | 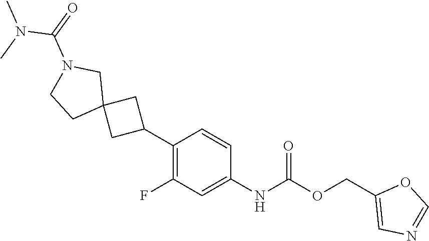 [SMILES: CN(C)C(=O)N1CCC2(CC(c3ccc(NC(=O)OCc4cnco4)cc3F)C2)C1] | |
| | Diastereoisomer 1 | |
| 364 | 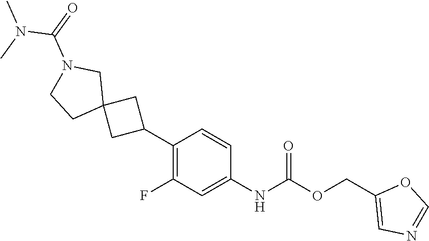 [SMILES: CN(C)C(=O)N1CCC2(CC(c3ccc(NC(=O)OCc4cnco4)cc3F)C2)C1] | |
| | Diastereoisomer 2 | |
| 365 | 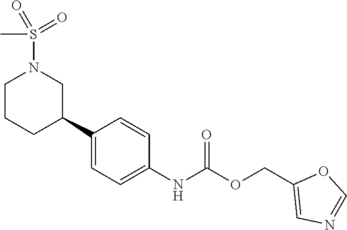 [SMILES: CS(=O)(=O)N1CCC[C@H](c2ccc(NC(=O)OCc3cnco3)cc2)C1] | oxazol-5-ylmethyl (R)- (4-(1- (methylsulfonyl)piperidin- 3-yl)phenyl)carbamate |
| 366 | 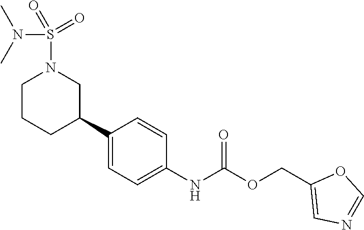 [SMILES: CN(C)S(=O)(=O)N1CCC[C@H](c2ccc(NC(=O)OCc3cnco3)cc2)C1] | oxazol-5-ylmethyl (R)- (4-(1-(N,N- dimethylsulfamoyl)piperidin-3- yl)phenyl)carbamate |
| 367 | 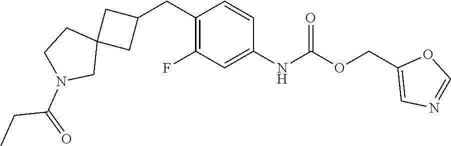 [SMILES: CCC(=O)N1CCC2(CC(Cc3ccc(NC(=O)OCc4cnco4)cc3F)C2)C1] | oxazol-5-ylmethyl (3- fluoro-4-((6-propionyl-6- azaspiro[3.4]octan-2- yl)methyl)phenyl)carbamate |
| 368 | 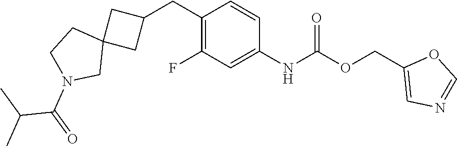 [SMILES: CC(C)C(=O)N1CCC2(CC(Cc3ccc(NC(=O)OCc4cnco4)cc3F)C2)C1] | oxazol-5-ylmethyl (3- fluoro-4-((6-isobutyryl-6- azaspiro[3.4]octan-2- yl)methyl)phenyl)carbamate |
| 369 | 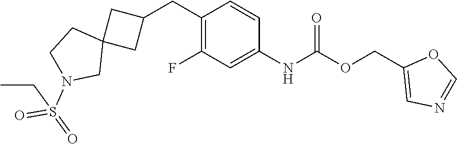 [SMILES: CCS(=O)(=O)N1CCC2(CC(Cc3ccc(NC(=O)OCc4cnco4)cc3F)C2)C1] | oxazol-5-ylmethyl (3- fluoro-4-((6- (ethylsulfonyl)-6- azaspiro[3.4]octan-2- yl)methyl)phenyl)carbamate |
| 370 | 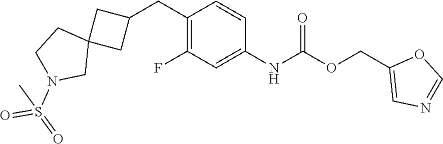 [SMILES: CS(=O)(=O)N1CCC2(CC(Cc3ccc(NC(=O)OCc4cnco4)cc3F)C2)C1] | oxazol-5-ylmethyl (3- fluoro-4-((6- (methylsulfonyl)-6- azaspiro[3.4]octan-2- yl)methyl)phenyl)carbamate |
| 371 | 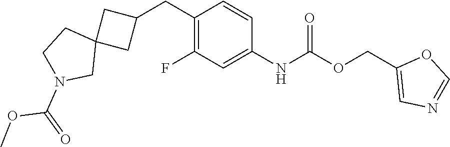 [SMILES: COC(=O)N1CCC2(CC(Cc3ccc(NC(=O)OCc4cnco4)cc3F)C2)C1] | methyl 2-(2-fluoro-4- (((oxazol-5- ylmethoxy)carbonyl)ami- no)benzyl)-6- azaspiro[3.4]octane-6- carboxylate |
| 372 | 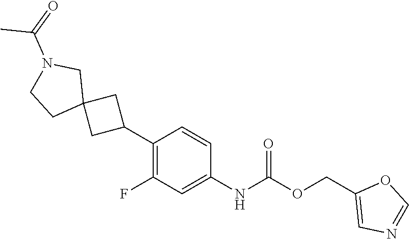 [SMILES: CC(=O)N1CCC2(CC(c3ccc(NC(=O)OCc4cnco4)cc3F)C2)C1] | |
| | Diastereoisomer 1 | |
| 373 | 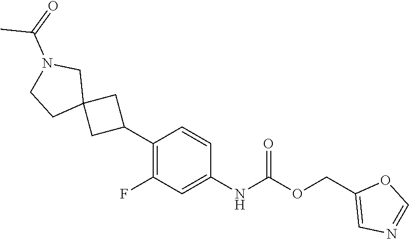 [SMILES: CC(=O)N1CCC2(CC(c3ccc(NC(=O)OCc4cnco4)cc3F)C2)C1] | |
| | Diastereoisomer 2 | |
| 374 | 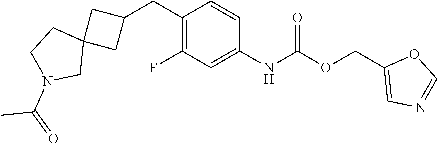 [SMILES: CC(=O)N1CCC2(CC(Cc3ccc(NC(=O)OCc4cnco4)cc3F)C2)C1] | oxazol-5-ylmethyl (4-((6- acetyl-6- azaspiro[3.4]octan-2- yl)methyl)-3- fluorophenyl)carbamate |
| 375 | 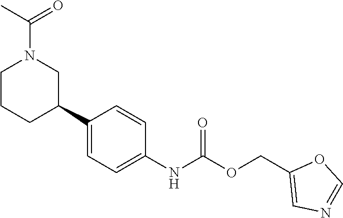 [SMILES: CC(=O)N1CCC[C@H](c2ccc(NC(=O)OCc3cnco3)cc2)C1] | oxazol-5-ylmethyl (R)- (4-(1-acetylpiperidin-3- yl)phenyl)carbamate |
| 376 | 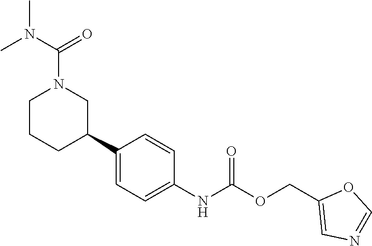 [SMILES: CN(C)C(=O)N1CCC[C@H](c2ccc(NC(=O)OCc3cnco3)cc2)C1] | oxazol-5-ylmethyl (R)- (4-(1- (dimethylcarbamoyl)piperidin-3- yl)phenyl)carbamate |
| 377 | 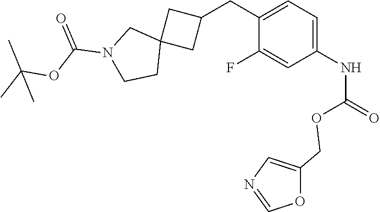 [SMILES: CC(C)(C)OC(=O)N1CCC2(CC(Cc3ccc(NC(=O)OCc4cnco4)cc3F)C2)C1] | tert-butyl 2-(2-fluoro-4- (((oxazol-5- ylmethoxy)carbonyl)ami- no)benzyl)-6- azaspiro[3.4]octane-6- carboxylate |
| 378 | 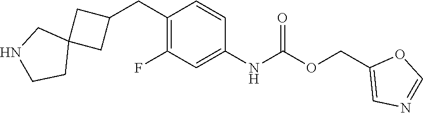 [SMILES: O=C(Nc1ccc(CC2CC3(CCNC3)C2)c(F)c1)OCc1cnco1] | oxazol-5-ylmethyl (4-((6- azaspiro[3.4]octan-2- yl)methyl)-3- fluorophenyl)carbamate |
| 379 | 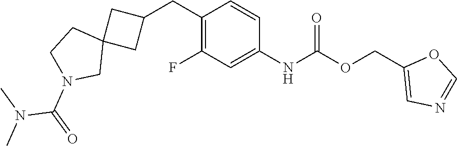 [SMILES: CN(C)C(=O)N1CCC2(CC(Cc3ccc(NC(=O)OCc4cnco4)cc3F)C2)C1] | oxazol-5-ylmethyl (4-((6- (dimethylcarbamoyl)-6- azaspiro[3.4]octan-2- yl)methyl)-3- fluorophenyl)carbamate |
| 380 | 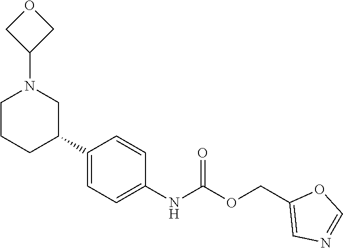 [SMILES: O=C(Nc1ccc([C@@H]2CCCN(C3COC3)C2)cc1)OCc1cnco1] | oxazol-5-ylmethyl (S)- (4-(1-(oxetan-3- yl)piperidin-3- yl)phenyl)carbamate |
| 381 | 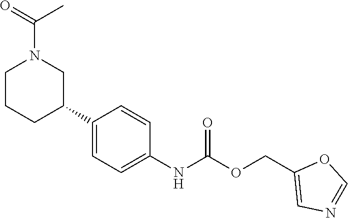 [SMILES: CC(=O)N1CCC[C@@H](c2ccc(NC(=O)OCc3cnco3)cc2)C1] | oxazol-5-ylmethyl (S)- (4-(1-acetylpiperidin-3- yl)phenyl)carbamate |
| 382 | 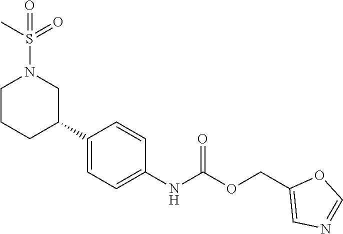 [SMILES: CS(=O)(=O)N1CCC[C@@H](c2ccc(NC(=O)OCc3cnco3)cc2)C1] | oxazol-5-ylmethyl (S)- (4-(1- (methylsulfonyl)piperidin- 3-yl)phenyl)carbamate |
| 383 | 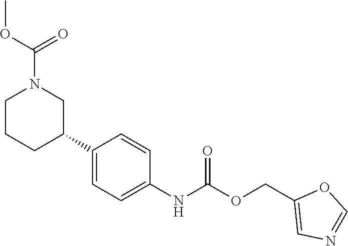 [SMILES: COC(=O)N1CCC[C@@H](c2ccc(NC(=O)OCc3cnco3)cc2)C1] | methyl (S)-3-(4- (((oxazol-5- ylmethoxy)carbonyl)ami- no)phenyl)piperidine-1- carboxylate |
| 384 | 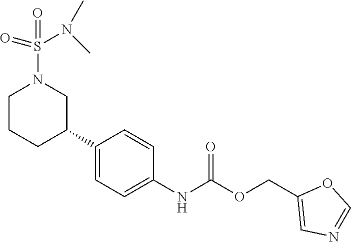 [SMILES: CN(C)S(=O)(=O)N1CCC[C@@H](c2ccc(NC(=O)OCc3cnco3)cc2)C1] | oxazol-5-ylmethyl (S)- (4-(1-(N,N-dimethyl- sulfamoyl)piperidin-3- yl)phenyl)carbamate |
| 385 | 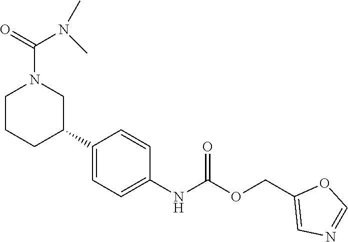 [SMILES: CN(C)C(=O)N1CCC[C@@H](c2ccc(NC(=O)OCc3cnco3)cc2)C1] | oxazol-5-ylmethyl (S)- (4-(1-(dimethyl- carbamoyl)piperidin-3- yl)phenyl)carbamate |
| 386 | 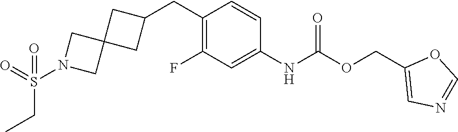 [SMILES: CCS(=O)(=O)N1CC2(CC(Cc3ccc(NC(=O)OCc4cnco4)cc3F)C2)C1] | oxazol-5-ylmethyl (4-((2- (ethylsulfonyl)-2- azaspiro[3.3]heptan-6- yl)methyl)-3- fluorophenyl)carbamate |
| 387 | 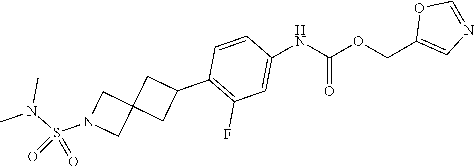 [SMILES: CN(C)S(=O)(=O)N1CC2(CC(c3ccc(NC(=O)OCc4cnco4)cc3F)C2)C1] | oxazol-5-ylmethyl (4-(2- (N,N- dimethylsulfamoyl)-2- azaspiro[3.3]heptan-6- yl)-3- fluorophenyl)carbamate |
| 388 | 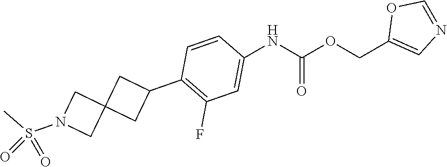 [SMILES: CS(=O)(=O)N1CC2(CC(c3ccc(NC(=O)OCc4cnco4)cc3F)C2)C1] | oxazol-5-ylmethyl (3- fluoro-4-(2- (methylsulfonyl)-2- azaspiro[3.3]heptan-6- yl)phenyl)carbamate |
| 389 | 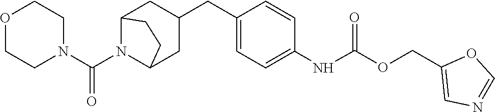 [SMILES: O=C(Nc1ccc(CC2CC3CCC(C2)N3C(=O)N2CCOCC2)cc1)OCc1cnco1] | oxazol-5-ylmethyl (4-((8- (morpholine-4-carbonyl)- 8-azabicyclo[3.2.1]octan-3- yl)methyl)phenyl)carbamate |
| 390 | 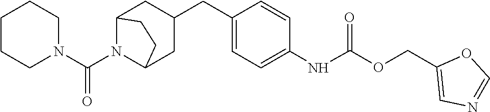 [SMILES: O=C(Nc1ccc(CC2CC3CCC(C2)N3C(=O)N2CCCCC2)cc1)OCc1cnco1] | oxazol-5-ylmethyl (4-((8- (piperidine-1-carbonyl)- 8-azabicyclo[3.2.1]octan-3- yl)methyl)phenyl)carbamate |
| 391 | 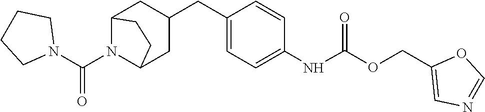 [SMILES: O=C(Nc1ccc(CC2CC3CCC(C2)N3C(=O)N2CCCC2)cc1)OCc1cnco1] | oxazol-5-ylmethyl (4-((8- (pyrrolidine-1-carbonyl)- 8-azabicyclo[3.2.1]octan-3- yl)methyl)phenyl)carbamate |
| 392 | 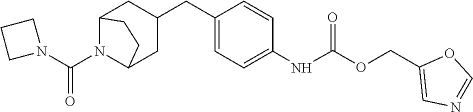 [SMILES: O=C(Nc1ccc(CC2CC3CCC(C2)N3C(=O)N2CCC2)cc1)OCc1cnco1] | oxazol-5-ylmethyl (4-((8- (azetidine-1-carbonyl)-8- azabicyclo[3.2.1]octan-3- yl)methyl)phenyl)carbamate |
| 393 | 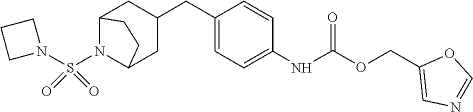 [SMILES: O=C(Nc1ccc(CC2CC3CCC(C2)N3S(=O)(=O)N2CCC2)cc1)OCc1cnco1] | oxazol-5-ylmethyl (4-((8- (azetidin-1-ylsulfonyl)-8- azabicyclo[3.2.1]octan-3- yl)methyl)phenyl)carbamate |
| 394 | 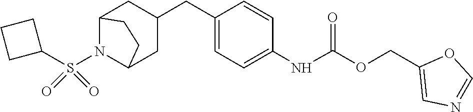 [SMILES: O=C(Nc1ccc(CC2CC3CCC(C2)N3S(=O)(=O)C2CCC2)cc1)OCc1cnco1] | oxazol-5-ylmethyl (4-((8- (cyclobutylsulfonyl)-8- azabicyclo[3.2.1]octan-3- yl)methyl)phenyl)carbamate |
| 395 | 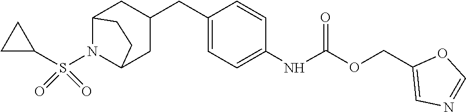 [SMILES: O=C(Nc1ccc(CC2CC3CCC(C2)N3S(=O)(=O)C2CC2)cc1)OCc1cnco1] | oxazol-5-ylmethyl (4-((8- (cyclopropylsulfonyl)-8- azabicyclo[3.2.1]octan-3- yl)methyl)phenyl)carbamate |
| 396 | 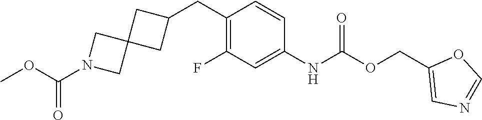 [SMILES: COC(=O)N1CC2(CC(Cc3ccc(NC(=O)OCc4cnco4)cc3F)C2)C1] | methyl 6-(2-fluoro-4- (((oxazol-5- ylmethoxy)carbonyl)ami- no)benzyl)-2- azaspiro[3.3]heptane-2- carboxylate |
| 397 | 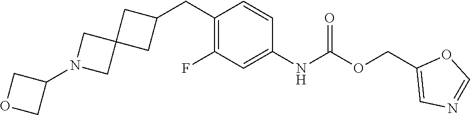 [SMILES: O=C(Nc1ccc(CC2CC3(C2)CN(C2COC2)C3)c(F)c1)OCc1cnco1] | oxazol-5-ylmethyl (3- fluoro-4-((2-(oxetan-3- yl)-2- azaspiro[3.3]heptan-6- yl)methyl)phenyl)carbamate |
| 398 | 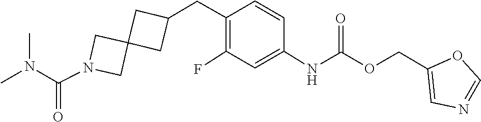 [SMILES: CN(C)C(=O)N1CC2(CC(Cc3ccc(NC(=O)OCc4cnco4)cc3F)C2)C1] | oxazol-5-ylmethyl (4-((2- (dimethylcarbamoyl)-2- azaspiro[3.3]heptan-6- yl)methyl)-3- fluorophenyl)carbamate |
| 399 | 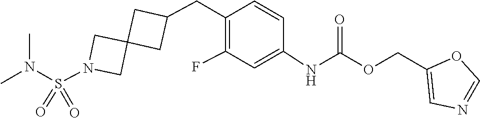 [SMILES: CN(C)S(=O)(=O)N1CC2(CC(Cc3ccc(NC(=O)OCc4cnco4)cc3F)C2)C1] | oxazol-5-ylmethyl (4-((2- (N,N- dimethylsulfamoyl)-2- azaspiro[3.3]heptan-6- yl)methyl)-3- fluorophenyl)carbamate |
| 400 | 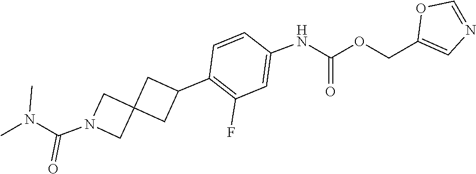 [SMILES: CN(C)C(=O)N1CC2(CC(c3ccc(NC(=O)OCc4cnco4)cc3F)C2)C1] | oxazol-5-ylmethyl (4-(2- (dimethylcarbamoyl)-2- azaspiro[3.3]heptan-6- yl)-3- fluorophenyl)carbamate |
| 401 | 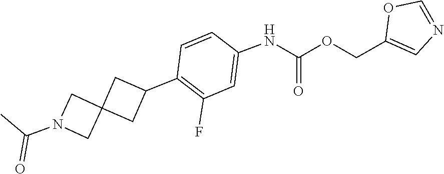 [SMILES: CC(=O)N1CC2(CC(c3ccc(NC(=O)OCc4cnco4)cc3F)C2)C1] | oxazol-5-ylmethyl (4-(2- acetyl-2- azaspiro[3.3]heptan-6- yl)-3- fluorophenyl)carbamate |
| 402 | 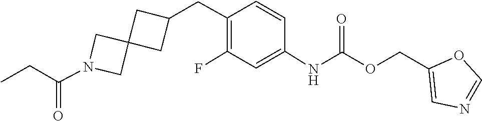 [SMILES: CCC(=O)N1CC2(CC(Cc3ccc(NC(=O)OCc4cnco4)cc3F)C2)C1] | oxazol-5-ylmethyl (3- fluoro-4-((2-propionyl-2- azaspiro[3.3]heptan-6- yl)methyl)phenyl)carbamate |
| 403 | 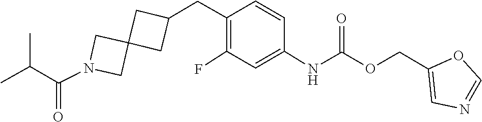 [SMILES: CC(C)C(=O)N1CC2(CC(Cc3ccc(NC(=O)OCc4cnco4)cc3F)C2)C1] | oxazol-5-ylmethyl (3- fluoro-4-((2-isobutyryl-2- azaspiro[3.3]heptan-6- yl)methyl)phenyl)carbamate |
| 404 | 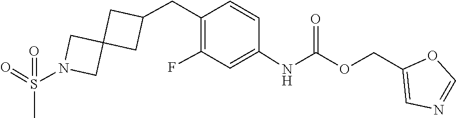 [SMILES: CS(=O)(=O)N1CC2(CC(Cc3ccc(NC(=O)OCc4cnco4)cc3F)C2)C1] | oxazol-5-ylmethyl (3- fluoro-4-((2- (methylsulfonyl)-2- azaspiro[3.3]heptan-6- yl)methyl)phenyl)carbamate |
| 405 | 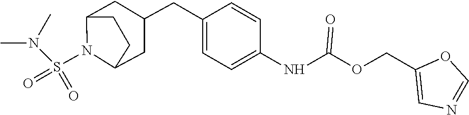 [SMILES: CN(C)S(=O)(=O)N1C2CCC1CC(Cc1ccc(NC(=O)OCc3cnco3)cc1)C2] | oxazol-5-ylmethyl (4-((8- (N,N- dimethylsulfamoyl)-8- azabicyclo[3.2.1]octan-3- yl)methyl)phenyl)carbamate |
| 406 | 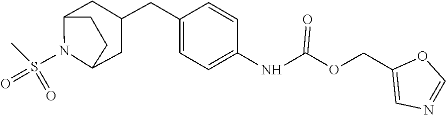 [SMILES: CS(=O)(=O)N1C2CCC1CC(Cc1ccc(NC(=O)OCc3cnco3)cc1)C2] | oxazol-5-ylmethyl (4-((8- (methylsulfonyl)-8- azabicyclo[3.2.1]octan-3- yl)methyl)phenyl)carbamate |
| 407 | 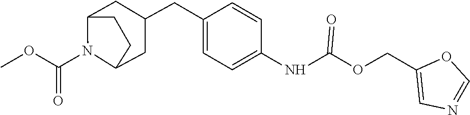 [SMILES: COC(=O)N1C2CCC1CC(Cc1ccc(NC(=O)OCc3cnco3)cc1)C2] | methyl 3-(4-(((oxazol-5- ylmethoxy)carbonyl)ami- no)benzyl)-8- azabicyclo[3.2.1]octane- 8-carboxylate |
| 408 | 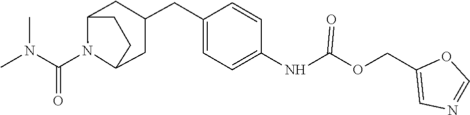 [SMILES: CN(C)C(=O)N1C2CCC1CC(Cc1ccc(NC(=O)OCc3cnco3)cc1)C2] | oxazol-5-ylmethyl (4-((8- (dimethylcarbamoyl)-8- azabicyclo[3.2.1]octan-3- yl)methyl)phenyl)carbamate |
| 409 | 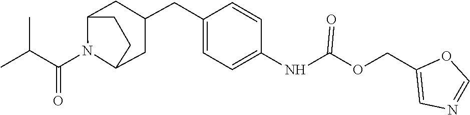 [SMILES: CC(C)C(=O)N1C2CCC1CC(Cc1ccc(NC(=O)OCc3cnco3)cc1)C2] | oxazol-5-ylmethyl (4-((8- isobutyryl-8- azabicyclo[3.2.1]octan-3- yl)methyl)phenyl)carbamate |
| 410 | 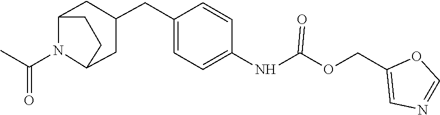 [SMILES: CC(=O)N1C2CCC1CC(Cc1ccc(NC(=O)OCc3cnco3)cc1)C2] | oxazol-5-ylmethyl (4-((8- acetyl-8- azabicyclo[3.2.1]octan-3- yl)methyl)phenyl)carbamate |
| 411 | 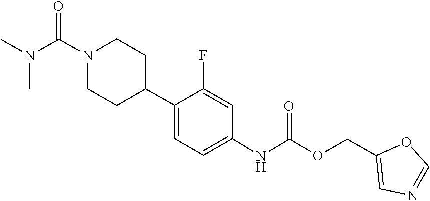 [SMILES: CN(C)C(=O)N1CCC(c2ccc(NC(=O)OCc3cnco3)cc2F)CC1] | oxazol-5-ylmethyl (4-(1- (dimethylcarbamoyl)piperidin- 4-yl)-3- fluorophenyl)carbamate |
| 412 | 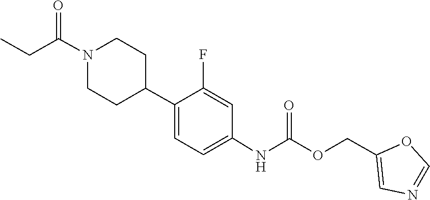 [SMILES: CCC(=O)N1CCC(c2ccc(NC(=O)OCc3cnco3)cc2F)CC1] | oxazol-5-ylmethyl (3- fluoro-4-(1- propionylpiperidin-4- yl)phenyl)carbamate |
| 413 | 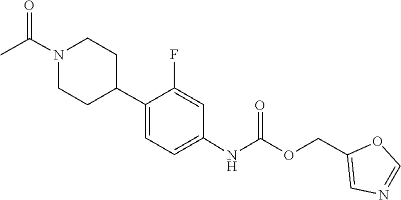 [SMILES: CC(=O)N1CCC(c2ccc(NC(=O)OCc3cnco3)cc2F)CC1] | oxazol-5-ylmethyl (4-(1- acetylpiperidin-4-yl)-3- fluorophenyl)carbamate |
| 414 | 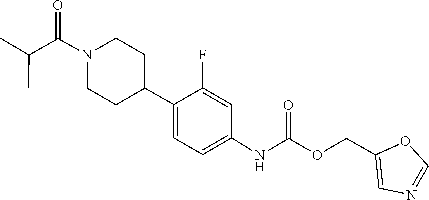 [SMILES: CC(C)C(=O)N1CCC(c2ccc(NC(=O)OCc3cnco3)cc2F)CC1] | oxazol-5-ylmethyl (3- fluoro-4-(1- isobutyrylpiperidin-4- yl)phenyl)carbamate |
| 415 | 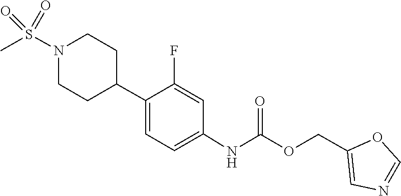 [SMILES: CS(=O)(=O)N1CCC(c2ccc(NC(=O)OCc3cnco3)cc2F)CC1] | oxazol-5-ylmethyl (3- fluoro-4-(1- (methylsulfonyl)piperidin- 4-yl)phenyl)carbamate |
| 416 | 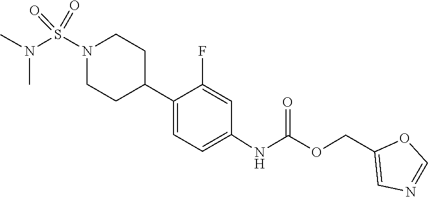 [SMILES: CN(C)S(=O)(=O)N1CCC(c2ccc(NC(=O)OCc3cnco3)cc2F)CC1] | oxazol-5-ylmethyl (4-(1- (N,N-dimethyl- sulfamoyl)piperidin- 4-yl)-3- fluorophenyl)carbamate |
| 417 | 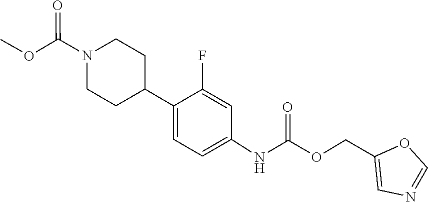 [SMILES: COC(=O)N1CCC(c2ccc(NC(=O)OCc3cnco3)cc2F)CC1] | methyl 4-(2-fluoro-4- (((oxazol-5- ylmethoxy)carbonyl)ami- no)phenyl)piperidine-1- carboxylate |
| 418 | 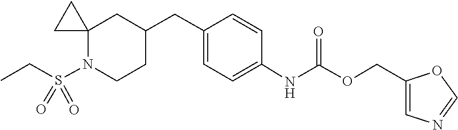 [SMILES: CCS(=O)(=O)N1CCC(Cc2ccc(NC(=O)OCc3cnco3)cc2)CC12CC2] | oxazol-5-ylmethyl (4-((4- (ethylsulfonyl)-4- azaspiro[2.5]octan-7- yl)methyl)phenyl)carbamate |
| 419 | 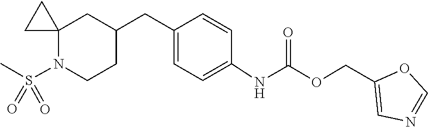 [SMILES: CS(=O)(=O)N1CCC(Cc2ccc(NC(=O)OCc3cnco3)cc2)CC12CC2] | oxazol-5-ylmethyl (4-((4- (methylsulfonyl)-4- azaspiro[2.5]octan-7- yl)methyl)phenyl)carbamate |
| 420 | 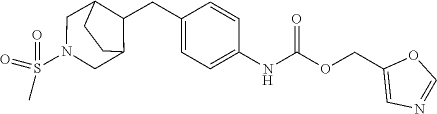 | oxazol-5-ylmethyl (4-((3- (methylsulfonyl)-3- azabicyclo[3.2.1]octan-8- yl)methyl)phenyl)carbamate |
| 421 | 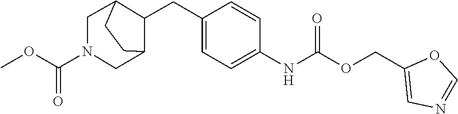 | methyl 8-(4-(((oxazol-5- ylmethoxy)carbonyl)ami- no)benzyl)-3- azabicyclo[3.2.1]octane- 3-carboxylate |
| 422 | 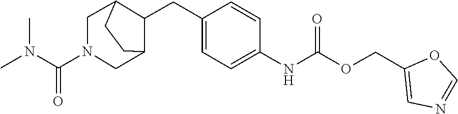 | oxazol-5-ylmethyl (4-((3- (dimethylcarbamoyl)-3- azabicyclo[3.2.1]octan-8- yl)methyl)phenyl)carbamate |
| 423 | 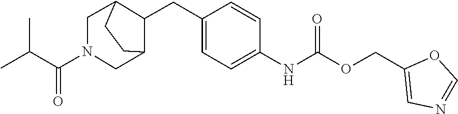 | oxazol-5-ylmethyl (4-((3- isobutyryl-3- azabicyclo[3.2.1]octan-8- yl)methyl)phenyl)carbamate |
| 424 | 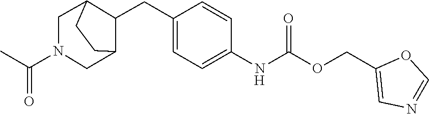 | oxazol-5-ylmethyl (4-((3- acetyl-3- azabicyclo[3.2.1]octan-8- yl)methyl)phenyl)carbamate |
| 425 | 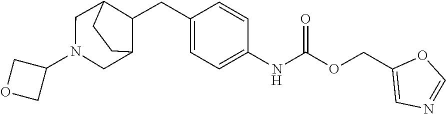 | oxazol-5-ylmethyl (4-((3- (oxetan-3-yl)-3- azabicyclo[3.2.1]octan-8- yl)methyl)phenyl)carbamate |
| 426 | 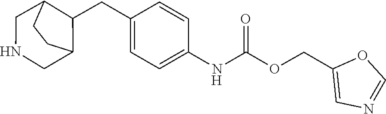 | oxazol-5-ylmethyl (4-((3- azabicyclo[3.2.1]octan-8- yl)methyl)phenyl)carbamate |
| 427 | 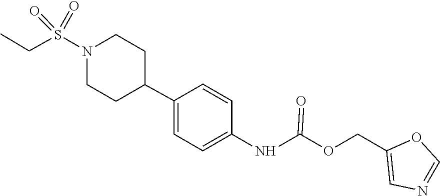 [SMILES: CCS(=O)(=O)N1CCC(c2ccc(NC(=O)OCc3cnco3)cc2)CC1] | oxazol-5-ylmethyl (4-(1- (ethylsulfonyl)piperidin- 4-yl)phenyl)carbamate |
| 428 | 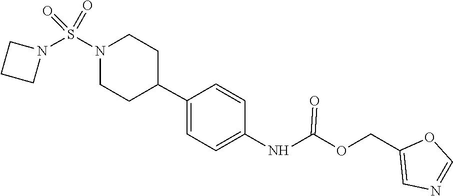 [SMILES: O=C(Nc1ccc(C2CCN(S(=O)(=O)N3CCC3)CC2)cc1)OCc1cnco1] | oxazol-5-ylmethyl (4-(1- (azetidin-1- ylsulfonyl)piperidin-4- yl)phenyl)carbamate |
| 429 | 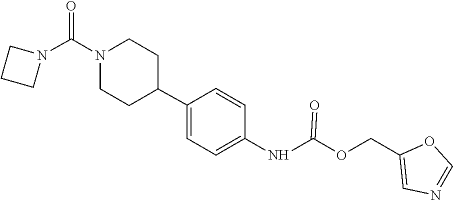 [SMILES: O=C(Nc1ccc(C2CCN(C(=O)N3CCC3)CC2)cc1)OCc1cnco1] | oxazol-5-ylmethyl (4-(1- (azetidine-1- carbonyl)piperidin-4- yl)phenyl)carbamate |
| 430 | 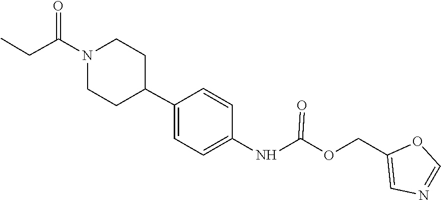 [SMILES: CCC(=O)N1CCC(c2ccc(NC(=O)OCc3cnco3)cc2)CC1] | oxazol-5-ylmethyl (4-(1- propionylpiperidin-4- yl)phenyl)carbamate |
| 431 | 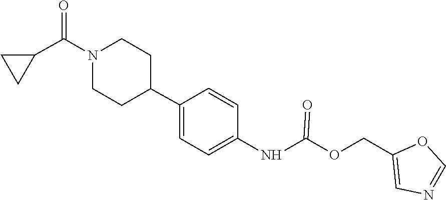 [SMILES: O=C(Nc1ccc(C2CCN(C(=O)C3CC3)CC2)cc1)OCc1cnco1] | oxazol-5-ylmethyl (4-(1- (cyclopropane- carbonyl)piperidin-4- yl)phenyl)carbamate |
| 432 | 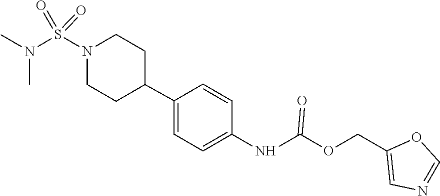 [SMILES: CN(C)S(=O)(=O)N1CCC(c2ccc(NC(=O)OCc3cnco3)cc2)CC1] | oxazol-5-ylmethyl (4-(1- (N,N-dimethyl- sulfamoyl)piperidin-4- yl)phenyl)carbamate |
| 433 | 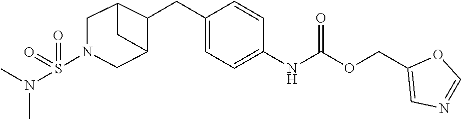 | oxazol-5-ylmethyl (4-((3- (N,N- dimethylsulfamoyl)-3- azabicyclo[3.1.1]heptan-6- yl)methyl)phenyl)carbamate |
| 434 | 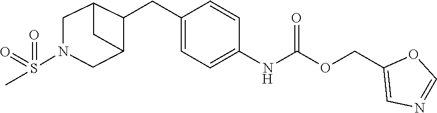 | oxazol-5-ylmethyl (4-((3- (methylsulfonyl)-3- azabicyclo[3.1.1]heptan-6- yl)methyl)phenyl)carbamate |
| 435 | 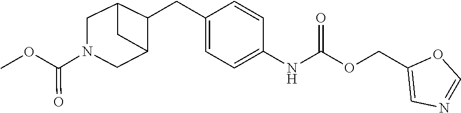 | methyl 6-(4-(((oxazol-5- ylmethoxy)carbonyl)ami- no)benzyl)-3- azabicyclo[3.1.1]heptane- 3-carboxylate |
| 436 | 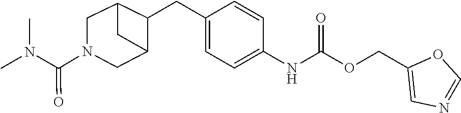 | oxazol-5-ylmethyl (4-((3- (dimethylcarbamoyl)-3- azabicyclo[3.1.1]heptan-6- yl)methyl)phenyl)carbamate |
| 437 | 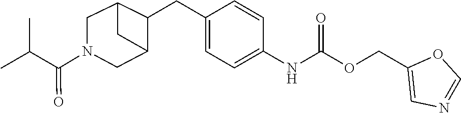 | oxazol-5-ylmethyl (4-((3- isobutyryl-3- azabicyclo[3.1.1]heptan-6- yl)methyl)phenyl)carbamate |
| 438 | 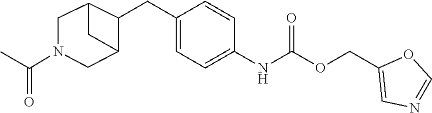 | oxazol-5-ylmethyl (4-((3- acetyl-3- azabicyclo[3.1.1]heptan-6- yl)methyl)phenyl)carbamate |
| 439 | 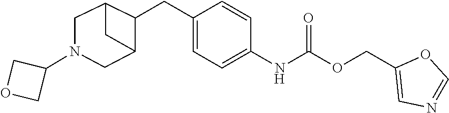 | oxazol-5-ylmethyl (4-((3- (oxetan-3-yl)-3- azabicyclo[3.1.1]heptan-6- yl)methyl)phenyl)carbamate |
| 440 | 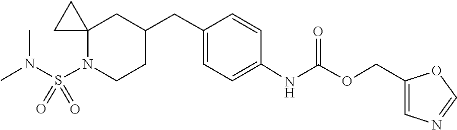 [SMILES: CN(C)S(=O)(=O)N1CCC(Cc2ccc(NC(=O)OCc3cnco3)cc2)CC12CC2] | oxazol-5-ylmethyl (4-((4- (N,N- dimethylsulfamoyl)-4- azaspiro[2.5]octan-7- yl)methyl)phenyl)carbamate |
| 441 | 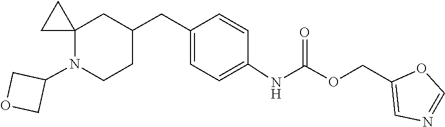 [SMILES: O=C(Nc1ccc(CC2CCN(C3COC3)C3(CC3)C2)cc1)OCc1cnco1] | oxazol-5-ylmethyl (4-((4- (oxetan-3-yl)-4- azaspiro[2.5]octan-7- yl)methyl)phenyl)carbamate |
| 442 | 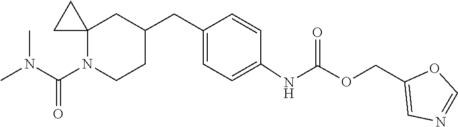 [SMILES: CN(C)C(=O)N1CCC(Cc2ccc(NC(=O)OCc3cnco3)cc2)CC12CC2] | oxazol-5-ylmethyl (4-((4- (dimethylcarbamoyl)-4- azaspiro[2.5]octan-7- yl)methyl)phenyl)carbamate |
| 443 | 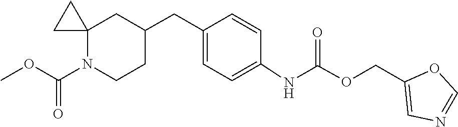 [SMILES: COC(=O)N1CCC(Cc2ccc(NC(=O)OCc3cnco3)cc2)CC12CC2] | methyl 7-(4-(((oxazol-5- ylmethoxy)carbonyl)ami- no)benzyl)-4- azaspiro[2.5]octane-4- carboxylate |
| 444 | 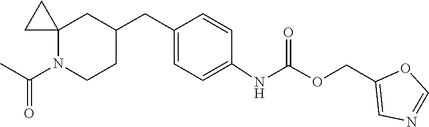 [SMILES: CC(=O)N1CCC(Cc2ccc(NC(=O)OCc3cnco3)cc2)CC12CC2] | oxazol-5-ylmethyl (4-((4- acetyl-4- azaspiro[2.5]octan-7- yl)methyl)phenyl)carbamate |
| 445 | 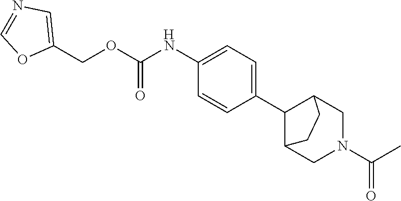 | oxazol-5-ylmethyl (4-(3- acetyl-3- azabicyclo[3.2.1]octan-8- yl)phenyl)carbamate |
| 446 | 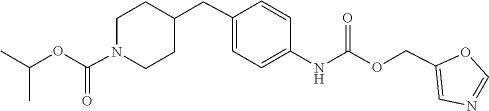 [SMILES: CC(C)OC(=O)N1CCC(Cc2ccc(NC(=O)OCc3cnco3)cc2)CC1] | isopropyl 4-(4-(((oxazol-5- ylmethoxy)carbonyl)ami- no)benzyl)piperidine-1- carboxylate |
| 447 | 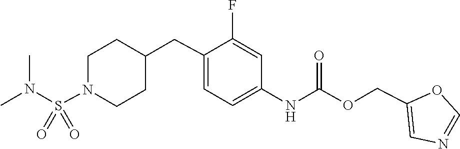 [SMILES: CN(C)S(=O)(=O)N1CCC(Cc2ccc(NC(=O)OCc3cnco3)cc2F)CC1] | oxazol-5-ylmethyl (4-((1- (N,N-dimethyl- sulfamoyl)piperidin- 4-yl)methyl)-3- fluorophenyl)carbamate |
| 448 | 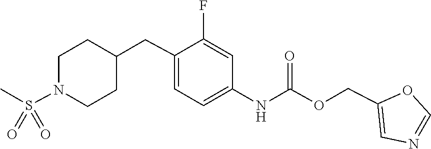 [SMILES: CS(=O)(=O)N1CCC(Cc2ccc(NC(=O)OCc3cnco3)cc2F)CC1] | oxazol-5-ylmethyl (3- fluoro-4-((1- (methylsulfonyl)piperidin-4- yl)methyl)phenyl)carbamate |
| 449 | 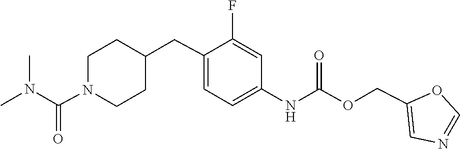 [SMILES: CN(C)C(=O)N1CCC(Cc2ccc(NC(=O)OCc3cnco3)cc2F)CC1] | oxazol-5-ylmethyl (4-((1- (dimethylcarbamoyl)piperidin- 4-yl)methyl)-3- fluorophenyl)carbamate |
| 450 | 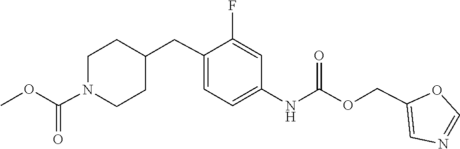 [SMILES: COC(=O)N1CCC(Cc2ccc(NC(=O)OCc3cnco3)cc2F)CC1] | methyl 4-(2-fluoro-4- (((oxazol-5- ylmethoxy)carbonyl)ami- no)benzyl)piperidine-1- carboxylate |
| 451 | 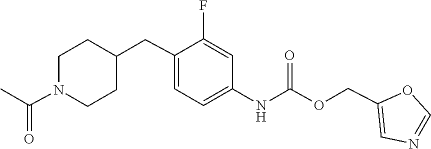 [SMILES: CC(=O)N1CCC(Cc2ccc(NC(=O)OCc3cnco3)cc2F)CC1] | oxazol-5-ylmethyl (4-((1- acetylpiperidin-4- yl)methyl)-3- fluorophenyl)carbamate |
| 452 | 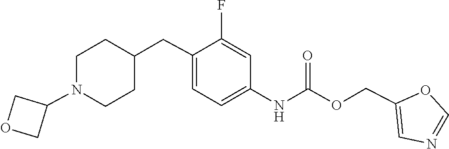 [SMILES: O=C(Nc1ccc(CC2CCN(C3COC3)CC2)c(F)c1)OCc1cnco1] | oxazol-5-ylmethyl (3- fluoro-4-((1-(oxetan-3- yl)piperidin-4- yl)methyl)phenyl)carbamate |
| 453 | 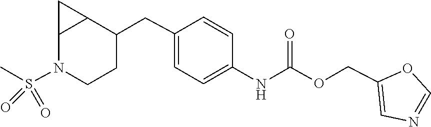 [SMILES: CS(=O)(=O)N1CCC(Cc2ccc(NC(=O)OCc3cnco3)cc2)C2CC21] | oxazol-5-ylmethyl (4-((2- (methylsulfonyl)-2- azabicyclo[4.1.0]heptan-5- yl)methyl)phenyl)carbamate |
| 454 | 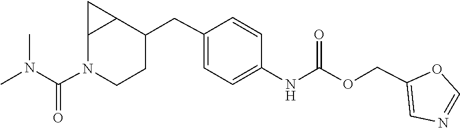 [SMILES: CN(C)C(=O)N1CCC(Cc2ccc(NC(=O)OCc3cnco3)cc2)C2CC21] | oxazol-5-ylmethyl (4-((2- (dimethylcarbamoyl)-2- azabicyclo[4.1.0]heptan-5- yl)methyl)phenyl)carbamate |
| 455 | 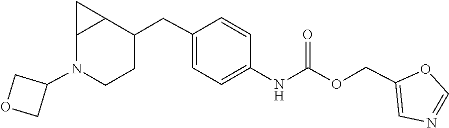 [SMILES: O=C(Nc1ccc(CC2CCN(C3COC3)C3CC23)cc1)OCc1cnco1] | oxazol-5-ylmethyl (4-((2- (oxetan-3-yl)-2- azabicyclo[4.1.0]heptan-5- yl)methyl)phenyl)carbamate |
| 456 | 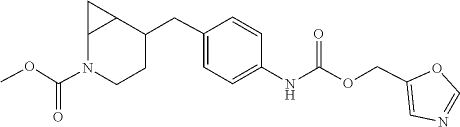 [SMILES: COC(=O)N1CCC(Cc2ccc(NC(=O)OCc3cnco3)cc2)C2CC21] | methyl 5-(4-(((oxazol-5- ylmethoxy)carbonyl)ami- no)benzyl)-2- azabicyclo[4.1.0]heptane- 2-carboxylate |
| 457 | 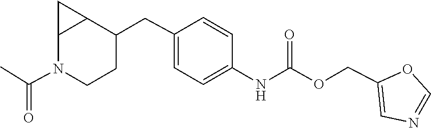 [SMILES: CC(=O)N1CCC(Cc2ccc(NC(=O)OCc3cnco3)cc2)C2CC21] | oxazol-5-ylmethyl (4-((2- acetyl-2- azabicyclo[4.1.0]heptan-5- yl)methyl)phenyl)carbamate |
| 458 | 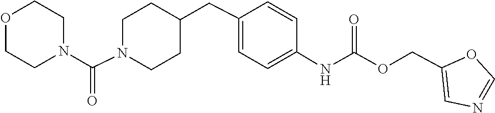 [SMILES: O=C(Nc1ccc(CC2CCN(C(=O)N3CCOCC3)CC2)cc1)OCc1cnco1] | oxazol-5-ylmethyl (4-((1- (morpholine-4- carbonyl)piperidin-4- yl)methyl)phenyl)carbamate |
| 459 | 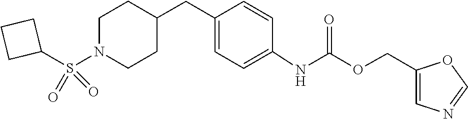 [SMILES: O=C(Nc1ccc(CC2CCN(S(=O)(=O)C3CCC3)CC2)cc1)OCc1cnco1] | oxazol-5-ylmethyl (4-((1- (cyclobutylsulfonyl)piperidin-4- yl)methyl)phenyl)carbamate |
| 460 | 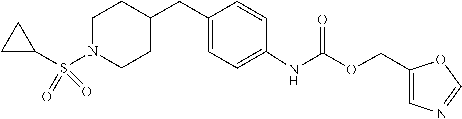 [SMILES: O=C(Nc1ccc(CC2CCN(S(=O)(=O)C3CC3)CC2)cc1)OCc1cnco1] | oxazol-5-ylmethyl (4-((1- (cyclopropylsul- fonyl)piperidin-4- yl)methyl)phenyl)carbamate |
| 461 | 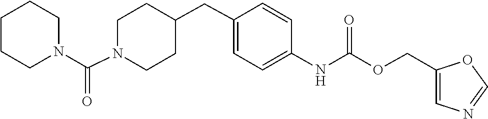 [SMILES: O=C(Nc1ccc(CC2CCN(C(=O)N3CCCCC3)CC2)cc1)OCc1cnco1] | oxazol-5-ylmethyl (4-((1- (piperidine-1- carbonyl)piperidin-4- yl)methyl)phenyl)carbamate |
| 462 | 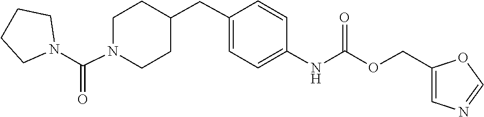 [SMILES: O=C(Nc1ccc(CC2CCN(C(=O)N3CCCC3)CC2)cc1)OCc1cnco1] | oxazol-5-ylmethyl (4-((1- (pyrrolidine-1- carbonyl)piperidin-4- yl)methyl)phenyl)carbamate |
| 463 | 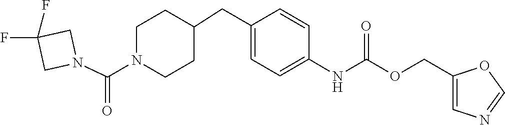 [SMILES: O=C(Nc1ccc(CC2CCN(C(=O)N3CC(F)(F)C3)CC2)cc1)OCc1cnco1] | oxazol-5-ylmethyl (4-((1- (3,3-difluoroazetidine-1- carbonyl)piperidin-4- yl)methyl)phenyl)carbamate |
| 464 | 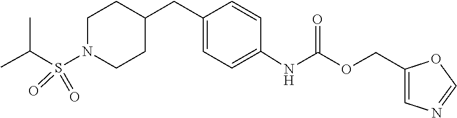 [SMILES: CC(C)S(=O)(=O)N1CCC(Cc2ccc(NC(=O)OCc3cnco3)cc2)CC1] | oxazol-5-ylmethyl (4-((1- (isopropylsulfonyl)piperidin-4- yl)methyl)phenyl)carbamate |
| 465 | 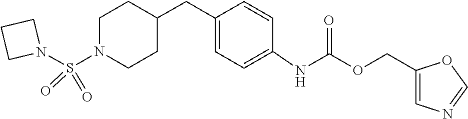 [SMILES: O=C(Nc1ccc(CC2CCN(S(=O)(=O)N3CCC3)CC2)cc1)OCc1cnco1] | oxazol-5-ylmethyl (4-((1- (azetidin-1- ylsulfonyl)piperidin-4- yl)methyl)phenyl)carbamate |
| 466 | 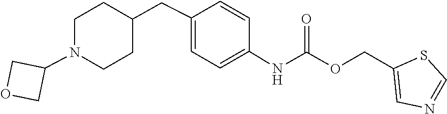 [SMILES: O=C(Nc1ccc(CC2CCN(C3COC3)CC2)cc1)OCc1cncs1] | thiazol-5-ylmethyl (4- ((1-(oxetan-3- yl)piperidin-4- yl)methyl)phenyl)carbamate |
| 467 | 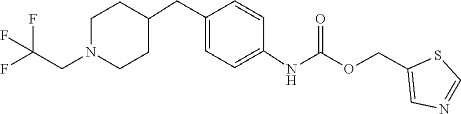 [SMILES: O=C(Nc1ccc(CC2CCN(CC(F)(F)F)CC2)cc1)OCc1cncs1] | thiazol-5-ylmethyl (4- ((1-(2,2,2- trifluoroethyl)piperidin-4- yl)methyl)phenyl)carbamate |
| 468 | 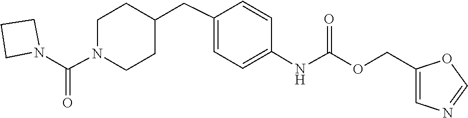 [SMILES: O=C(Nc1ccc(CC2CCN(C(=O)N3CCC3)CC2)cc1)OCc1cnco1] | oxazol-5-ylmethyl (4-((1- (azetidine-1- carbonyl)piperidin-4- yl)methyl)phenyl)carbamate |
| 469 | 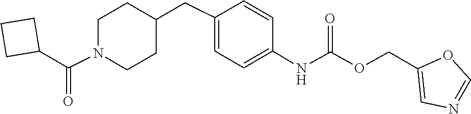 [SMILES: O=C(Nc1ccc(CC2CCN(C(=O)C3CCC3)CC2)cc1)OCc1cnco1] | oxazol-5-ylmethyl (4-((1- (cyclobutane- carbonyl)piperidin-4- yl)methyl)phenyl)carbamate |
| 470 | 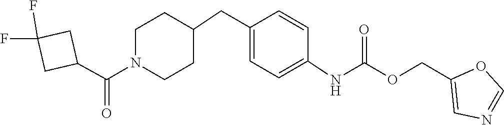 [SMILES: O=C(Nc1ccc(CC2CCN(C(=O)C3CC(F)(F)C3)CC2)cc1)OCc1cnco1] | oxazol-5-ylmethyl (4-((1- (3,3-difluorocyclobutane- 1-carbonyl)piperidin-4- yl)methyl)phenyl)carbamate |
| 471 | 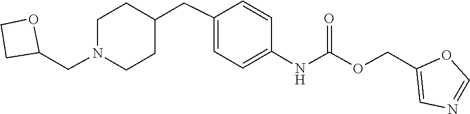 [SMILES: O=C(Nc1ccc(CC2CCN(CC3CCO3)CC2)cc1)OCc1cnco1] | oxazol-5-ylmethyl (4-((1- (oxetan-2- ylmethyl)piperidin-4- yl)methyl)phenyl)carbamate |
| 472 | 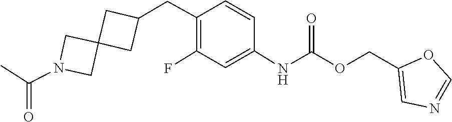 [SMILES: CC(=O)N1CC2(CC(Cc3ccc(NC(=O)OCc4cnco4)cc3F)C2)C1] | oxazol-5-ylmethyl (4-((2- acetyl-2- azaspiro[3.3]heptan-6- yl)methyl)-3- fluorophenyl)carbamate |

In some embodiments, provided herein is a compound of Formula (I), or any variation thereof, or a pharmaceutically acceptable salt of any of the foregoing, selected from the group consisting of:
4-methoxybenzyl (4-((2-(pyridin-3-yl)pyrrolidin-1-yl)methyl)phenyl)carbamate;
4-carbamoylbenzyl (4-((2-(pyridin-3-yl)pyrrolidin-1-yl)methyl)phenyl)carbamate; pyridin-4-ylmethyl (4-((2-(pyridin-3-yl)pyrrolidin-1-yl)methyl)phenyl)carbamate;
4-(hydroxymethyl)benzyl (4-((2-(pyridin-3-yl)pyrrolidin-1-yl)methyl)phenyl)carbamate; oxazol-5-ylmethyl (4-((2-(pyridin-3-yl)pyrrolidin-1-yl)methyl)phenyl)carbamate;
4-chlorobenzyl (4-(1-(1,3,4-trimethyl-1H-pyrazole-5-carboxamido)ethyl)phenyl)carbamate;
4-chlorobenzyl (4-(1-(1-(tetrahydro-2H-pyran-4-yl)-1H-pyrazole-5-carboxamido)ethyl)phenyl)carbamate;
oxazol-5-ylmethyl (4-(1-(6,6-difluorospiro[3.3]heptane-2-carboxamido)ethyl)phenyl)carbamate;
oxazol-5-ylmethyl (4-(1-(4-fluorobenzamido)ethyl)phenyl)carbamate;
4-chlorobenzyl (4-(1-(3-fluoroisonicotinamido)ethyl)phenyl)carbamate;
4-chlorobenzyl (4-(1-(3,4-dimethylisoxazole-5-carboxamido)ethyl)phenyl)carbamate;
4-methoxybenzyl (4-(1-(2-(hydroxymethyl)pyrrolidin-1-yl)-1-oxopropan-2-yl)phenyl)carbamate;
4-chlorobenzyl (4-(1-methyl-5-oxopiperazin-2-yl)phenyl)carbamate;
4-chlorobenzyl (4-(1-(1-(2-methoxyethyl)-1H-pyrazole-5-carboxamido)ethyl)phenyl)carbamate;
4-chlorobenzyl (4-(1-(3,5-difluoroisonicotinamido)ethyl)phenyl)carbamate;
4-chlorobenzyl (4-(1-(6-methylnicotinamido)ethyl)phenyl)carbamate;
4-chlorobenzyl (4-(2-((4-methoxycyclohexyl)amino)-2-oxoethyl)phenyl)carbamate;
oxazol-5-ylmethyl (4-(1-(6,6-difluorobicyclo[3.1.0]hexane-3-carboxamido)ethyl)phenyl)carbamate;
4-chlorobenzyl (4-(1-(4-methoxypiperidine-1-carboxamido)ethyl)phenyl)carbamate;
oxazol-5-ylmethyl (4-(1-(3,3-difluorocyclobutane-1-carboxamido)ethyl)phenyl)carbamate;
4-chlorobenzyl (4-(1-(6-ethylnicotinamido)ethyl)phenyl)carbamate;
4-chloro-2-fluorobenzyl (4-(1-methyl-5-oxopiperazin-2-yl)phenyl)carbamate;
4-chlorobenzyl (4-(1-(N-methylazetidine-1-carboxamido)ethyl)phenyl)carbamate;
4-chlorobenzyl (4-(1-(4-methoxy-1-methyl-1H-pyrazole-5-carboxamido)ethyl)phenyl)carbamate;
4-chlorobenzyl (4-(1-(3-methoxy-1-methyl-1H-pyrazole-5-carboxamido)ethyl)phenyl)carbamate;
4-chlorobenzyl (4-(1-(azetidine-1-carboxamido)ethyl)phenyl)carbamate;
oxazol-5-ylmethyl (4-(1-(3,3-difluorocyclopentane-1-carboxamido)ethyl)phenyl)carbamate;
4-chlorobenzyl (4-(1-(6-methoxynicotinamido)ethyl)phenyl)carbamate;
4-chlorobenzyl (4-(1-(1-ethyl-1H-pyrazole-5-carboxamido)ethyl)phenyl)carbamate;
4-chlorobenzyl (4-(1-(pyrrolidine-1-carboxamido)ethyl)phenyl)carbamate;
4-chlorobenzyl (4-(1-(isonicotinamido)ethyl)phenyl)carbamate;
4-chlorobenzyl (4-(1-(5-methoxy-6-methylnicotinamido)ethyl)phenyl)carbamate;
4-chlorobenzyl (4-(1-(tetrahydro-2H-pyran-4-carboxamido)ethyl)phenyl)carbamate;
4-chlorobenzyl (4-(1-(nicotinamido)ethyl)phenyl)carbamate;
4-chlorobenzyl (4-(1-(oxetane-3-carboxamido)ethyl)phenyl)carbamate;
4-chlorobenzyl (4-(1-(2,4-dimethyloxazole-5-carboxamido)ethyl)phenyl)carbamate;
4-chlorobenzyl (4-(1-(1-isobutyl-1H-pyrazole-5-carboxamido)ethyl)phenyl)carbamate;
4-chlorobenzyl (4-(1-(4-methoxy-N-methylpiperidine-1-carboxamido)ethyl)phenyl)carbamate;
4-chlorobenzyl (4-(1-(3-ethyl-1-methyl-1H-pyrazole-5-carboxamido)ethyl)phenyl)carbamate;
4-chlorobenzyl (4-(1-(1,3-dimethyl-1H-pyrazole-5-carboxamido)ethyl)phenyl)carbamate;
4-chlorobenzyl (4-(1-(4-fluoro-1,3-dimethyl-1H-pyrazole-5-carboxamido)ethyl)phenyl)carbamate;
4-chlorobenzyl (4-(1-(4-methylisoxazole-5-carboxamido)ethyl)phenyl)carbamate;
4-chlorobenzyl (4-(1-(N-methylpyrrolidine-1-carboxamido)ethyl)phenyl)carbamate;
4-chlorobenzyl (4-(1-(3-methylazetidine-1-carboxamido)ethyl)phenyl)carbamate;
4-chlorobenzyl (4-(1-(4-methylpiperidine-1-carboxamido)ethyl)phenyl)carbamate;
4-chlorobenzyl (4-(1-(1-methyl-1H-pyrazole-5-carboxamido)ethyl)phenyl)carbamate;
4-chlorobenzyl (4-(1-(3-methylisoxazole-4-carboxamido)ethyl)phenyl)carbamate;
4-chlorobenzyl (4-(1-(4-methyloxazole-5-carboxamido)ethyl)phenyl)carbamate;
4-chlorobenzyl (4-(1-(morpholine-4-carboxamido)ethyl)phenyl)carbamate;
4-chlorobenzyl (4-(1-(5-fluoronicotinamido)ethyl)phenyl)carbamate;
4-chlorobenzyl (4-(2-(3-methoxypiperidin-1-yl)-2-oxoethyl)phenyl)carbamate;
4-chlorobenzyl (4-(1-(6-isopropylnicotinamido)ethyl)phenyl)carbamate;
4-chlorobenzyl (4-(1-(6-(difluoromethyl)nicotinamido)ethyl)phenyl)carbamate;
4-chlorobenzyl (4-(1-(3-methoxyazetidine-1-carboxamido)ethyl)phenyl)carbamate;
4-chlorobenzyl (4-(2-(3-hydroxypiperidin-1-yl)-2-oxoethyl)phenyl)carbamate;
4-chlorobenzyl (4-(1-(1-isopropyl-1H-pyrazole-5-carboxamido)ethyl)phenyl)carbamate;
4-chlorobenzyl (4-(1-(3-methyloxetane-3-carboxamido)ethyl)phenyl)carbamate;
4-chlorobenzyl (4-(1-(3-methylisoxazole-5-carboxamido)ethyl)phenyl)carbamate;
4-chlorobenzyl (4-(2-(2-(hydroxymethyl)pyrrolidin-1-yl)-2-oxoethyl)phenyl)carbamate;
4-(difluoromethyl)benzyl (4-(1-methyl-5-oxopiperazin-2-yl)phenyl)carbamate;
4-chlorobenzyl (4-(1-(1-cyclobutyl-1H-pyrazole-5-carboxamido)ethyl)phenyl)carbamate;
4-chlorobenzyl (4-(1-(2-methoxy-6-methylnicotinamido)ethyl)phenyl)carbamate;
4-chlorobenzyl (4-(1-(2-methyloxazole-5-carboxamido)ethyl)phenyl)carbamate;
4-chlorobenzyl (4-(2-((-4-(difluoromethoxy)cyclohexyl)amino)-2-oxoethyl)phenyl)carbamate;
4-chlorobenzyl (4-(1-(2-(oxazol-2-yl)acetamido)ethyl)phenyl)carbamate;
4-chlorobenzyl (4-(((1-methyl-2-oxopyrrolidin-3-yl)amino)methyl)phenyl)carbamate;
4-chlorobenzyl (4-(2-(3-methoxypiperidin-1-yl)-2-oxoethyl)phenyl)carbamate;
oxazol-5-ylmethyl (4-(1-(6,6-difluorobicyclo[3.1.0]hexane-3-carboxamido)ethyl)phenyl)carbamate;
4-chlorobenzyl (4-(1-cyclobutyl-5-oxopiperazin-2-yl)phenyl)carbamate;
4-chlorobenzyl (4-(1-(5-methyloxazole-4-carboxamido)ethyl)phenyl)carbamate;
4-chlorobenzyl (4-(1-(4-methyloxazole-2-carboxamido)ethyl)phenyl)carbamate;
4-chlorobenzyl (4-(2-(3-methylpyrrolidin-1-yl)-2-oxoethyl)phenyl)carbamate;
4-chlorobenzyl (4-(1-(oxazole-5-carboxamido)ethyl)phenyl)carbamate;
4-chlorobenzyl (4-((2-oxo-5-(pyridin-3-yl)pyrrolidin-1-yl)methyl)phenyl)carbamate;
4-chlorobenzyl (4-(2-(3-methylpiperidin-1-yl)-2-oxoethyl)phenyl)carbamate;
4-chlorobenzyl (4-(2-(2-(hydroxymethyl)piperidin-1-yl)-2-oxoethyl)phenyl)carbamate;
4-chlorobenzyl (4-(1-(3-(difluoromethyl)-1-methyl-1H-pyrazole-5-carboxamido)ethyl)phenyl)carbamate;
4-chlorobenzyl (4-(1-(4-ethyloxazole-5-carboxamido)ethyl)phenyl)carbamate;
4-chlorobenzyl (4-(2-(3-methylpiperidin-1-yl)-2-oxoethyl)phenyl)carbamate;
4-methoxybenzyl (4-(1-methyl-5-oxopiperazin-2-yl)phenyl)carbamate;
oxazol-5-ylmethyl (4-(1-(cyclopropanecarboxamido)ethyl)phenyl)carbamate;
4-chlorobenzyl (4-(1-(5-methyloxazole-2-carboxamido)ethyl)phenyl)carbamate;
4-fluorobenzyl (4-(1-methyl-5-oxopiperazin-2-yl)phenyl)carbamate;
4-chlorobenzyl (4-(2-((-4-methoxycyclohexyl)amino)-2-oxoethyl)phenyl)carbamate;
4-chlorobenzyl (4-(2-(2-(hydroxymethyl)piperidin-1-yl)-2-oxoethyl)phenyl)carbamate;
4-chlorobenzyl (4-(1-(4-cyclopropyloxazole-5-carboxamido)ethyl)phenyl)carbamate;
4-chlorobenzyl (4-(1-isopropyl-5-oxopiperazin-2-yl)phenyl)carbamate;
4-chlorobenzyl (4-(2-(3-hydroxypyrrolidin-1-yl)-2-oxoethyl)phenyl)carbamate;
4-chlorobenzyl (4-(2-(3-hydroxypyrrolidin-1-yl)-2-oxoethyl)phenyl)carbamate;
4-chlorobenzyl (4-(N-methyl-N-(tetrahydrofuran-3-yl)sulfamoyl)phenyl)carbamate;
4-chlorobenzyl (4-(2-oxo-2-((4-(trifluoromethoxy)cyclohexyl)amino)ethyl)phenyl)carbamate;
4-methoxybenzyl (4-(1-(2-(hydroxymethyl)pyrrolidin-1-yl)-1-oxopropan-2-yl)phenyl)carbamate;
oxazol-5-ylmethyl (3-fluoro-4-(6-(methylsulfonyl)-6-azaspiro[3.4]octan-2-yl)phenyl)carbamate;
oxazol-5-ylmethyl (3-fluoro-4-(6-isobutyryl-6-azaspiro[3.4]octan-2-yl)phenyl)carbamate;
oxazol-5-ylmethyl (4-(6-(dimethylcarbamoyl)-6-azaspiro[3.4]octan-2-yl)-3-fluorophenyl)carbamate;
oxazol-5-ylmethyl (4-(1-(methylsulfonyl)piperidin-3-yl)phenyl)carbamate;
oxazol-5-ylmethyl (4-(1-(N,N-dimethylsulfamoyl)piperidin-3-yl)phenyl)carbamate;
oxazol-5-ylmethyl (3-fluoro-4-((6-propionyl-6-azaspiro[3.4]octan-2-yl)methyl)phenyl)carbamate;
oxazol-5-ylmethyl (3-fluoro-4-((6-isobutyryl-6-azaspiro[3.4]octan-2-yl)methyl)phenyl)carbamate;
oxazol-5-ylmethyl (3-fluoro-4-((6-(ethylsulfonyl)-6-azaspiro[3.4]octan-2-yl)methyl)phenyl)carbamate;
oxazol-5-ylmethyl (3-fluoro-4-((6-(methylsulfonyl)-6-azaspiro[3.4]octan-2-yl)methyl)phenyl)carbamate;
methyl 2-(2-fluoro-4-(((oxazol-5-ylmethoxy)carbonyl)amino)benzyl)-6-azaspiro[3.4]octane-6-carboxylate;
oxazol-5-ylmethyl (4-(6-acetyl-6-azaspiro[3.4]octan-2-yl)-3-fluorophenyl)carbamate;
oxazol-5-ylmethyl (4-((6-acetyl-6-azaspiro[3.4]octan-2-yl)methyl)-3-fluorophenyl)carbamate;
oxazol-5-ylmethyl (4-(1-acetylpiperidin-3-yl)phenyl)carbamate;
tert-butyl 2-(2-fluoro-4-(((oxazol-5-ylmethoxy)carbonyl)amino)benzyl)-6-azaspiro[3.4]octane-6-carboxylate;
oxazol-5-ylmethyl (4-((6-azaspiro[3.4]octan-2-yl)methyl)-3-fluorophenyl)carbamate;
oxazol-5-ylmethyl (4-((6-(dimethylcarbamoyl)-6-azaspiro[3.4]octan-2-yl)methyl)-3-fluorophenyl)carbamate;
oxazol-5-ylmethyl (4-(1-(oxetan-3-yl)piperidin-3-yl)phenyl)carbamate;
methyl 3-(4-(((oxazol-5-ylmethoxy)carbonyl)amino)phenyl)piperidine-1-carboxylate;
oxazol-5-ylmethyl (4-(1-(dimethylcarbamoyl)piperidin-3-yl)phenyl)carbamate; and
4-chlorobenzyl (4-(1-(oxetan-3-yl)-5-oxopiperazin-2-yl)phenyl)carbamate,
   or a pharmaceutically acceptable salt of any of the foregoing.

In some variations, any of the compounds described herein, such as a compound of Formula (I), or any variation thereof, or a compound of Table 1 may be deuterated (e.g., one or more hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art.

Formula (I) is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 has two or more stereocenters, also provided are any enantiomer or diastereomer of the compound. For example, if a compound of Table 1 contains a first stereocenter and a second stereocenter with “R” and “R” stereochemical configurations, respectively, also provided are stereoisomers of the compound having first and second stereocenters with “S” and “S” stereochemical configurations, respectively, “S” and “R” stereochemical configurations, respectively, and “R” and “S” stereochemical configurations, respectively. If a compound of Table 1 contains a first stereocenter and a second stereocenter with “S” and “S” stereochemical configurations, respectively, also provided are stereoisomers of the compound having first and second stereocenters with “R” and “R” stereochemical configurations, respectively, “S” and “R” stereochemical configurations, respectively, and “R” and “S” stereochemical configurations, respectively. If a compound of Table 1 contains a first stereocenter and a second stereocenter with “S” and “R” stereochemical configurations, respectively, also provided are stereoisomers of the compound having first and second stereocenters with “R” and “S” stereochemical configurations, respectively, “R” and “R” stereochemical configurations, respectively, and “S” and “S” stereochemical configurations, respectively. Similarly, if a compound of Table 1 contains a first stereocenter and a second stereocenter with “R” and “S” stereochemical configurations, respectively, also provided are stereoisomers of the compound having first and second stereocenters with “S” and “R” stereochemical configurations, respectively, “R” and “R” stereochemical configurations, respectively, and “S” and “S” stereochemical configurations, respectively. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual or subject.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual or subject. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of Ring A, Ring B, L, R^{A}, R^{B}, R^{C}, R^{D}, m, n, p, R^{A1}R^{A2}, R^{L}, R^{C1}, R^{B1}, and R^{B2} as provided herein can be combined with every other variation or embodiment of Ring A, Ring B, L, R^{A}, R^{B}, R^{C}, R^{D}, m, n, p, R^{A1}, R^{A2}, R^{L}, R^{C1}, R^{B1}, and R^{B2}, the same as if each combination had been individually and specifically described.

Other embodiments will be apparent to those skilled in the art from the following detailed description.

As used herein, when any variable occurs more than one time in a chemical formula, its definition on each occurrence is independent of its definition at every other occurrence.

The compound names provided herein, including in Table 1, are provided by ChemBioDraw Professional. One of skilled in the art would understand that the compounds may be named or identified using various commonly recognized nomenclature systems and symbols. By way of example, the compounds may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

### Compositions

Also provided are compositions, such as pharmaceutical compositions, that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, provided are compositions, such as pharmaceutical compositions that contain one or more compounds described herein, or a pharmaceutically acceptable salt thereof.

In some embodiments, provided is a pharmaceutically acceptable composition comprising a compound of Formula (I), or a compound of Table 1, or a pharmaceutically acceptable salt thereof. In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds or conjugates that are substantially pure.

Also provided are packaged pharmaceutical compositions, comprising a pharmaceutical composition as described herein and instructions for using the composition to treat a patient suffering from a disease or condition described herein.

### Methods of Use

Compounds and compositions detailed herein, such as a pharmaceutical composition comprising a compound of any formula provided herein, or a pharmaceutically acceptable salt thereof, and a pharmaceutically acceptable carrier or excipient, may be used in methods of administration and treatment as provided herein.

Without being bound by theory, the compounds and pharmaceutical compositions disclosed herein are believed to act by modulating nicotinamide phosphoribosyltransferase (NAMPT). In some embodiments, the compounds and pharmaceutical compositions disclosed herein are activators of NAMPT. In some embodiments, provided are methods of treating a disease or condition mediated by NAMPT activity in an individual or subject, comprising administering to the individual or subject in need thereof a compound of Formula (I), or a compound of Table 1, or a pharmaceutically acceptable salt thereof. In some embodiments, provided are methods of treating cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder in an individual or subject, comprising administering to the individual or subject in need thereof a compound of Formula (I), or a compound of Table 1, or a pharmaceutically acceptable salt thereof. In some embodiments, the compound of Formula (I), or a compound of Table 1, or a pharmaceutically acceptable salt thereof, are capable of CNS penetration and have the ability to boost NAD levels in the brain for potential treatment of CNS diseases.

In some embodiments, the compounds and pharmaceutical compositions disclosed herein may have the ability to s prevent a disease or disorder (i.e., causing the clinical symptoms of the disease or disorder not to develop). In some embodiments, this encompasses situations where the disease or disorder is not currently being experienced but is expected to arise. In some embodiments, when used in a prophylactic manner, the compounds and pharmaceutical compositions disclosed and/or described herein may prevent a disease or disorder from developing or lessen the extent of a disease or disorder that may develop.

In some embodiments, provided are methods of treating a disease or condition, comprising administering to the individual or subject in need thereof a compound of Formula (I), or a compound of Table 1, or a pharmaceutically acceptable salt thereof. In some embodiments, the disease or condition is selected from the group consisting of:
Hereditary motor and sensory neuropathies including Charcot-Marie Tooth Disease and hereditary sensory neuropathy type I, and the subset of fatty acid oxidation disorders that induce peripheral neuropathy;
Mitochondrial encephalomyopathies (including but not limited to Leber Hereditary Optic Neuropathy, MELAS, Leigh Syndrome, Chronic Progressive External Opthalmoplegia, Kearn's Sayre Syndrome, Alper's Disease, Autosomal Dominant Optic Atrophy, Friedreich's Ataxia, and Congenital Lactic Acidosis);
Concussion;
Ataxia Telangectasia;
Dysfunction of a variety of tissues including the central and peripheral nervous system, muscle, and immune system following viral infection including but not limited to SARS-CoV-2;
Treatment of obesity by raising NAD+ in the hypothalamus and peripheral tissues;
Treatment of hypertension by raising NAD+ systemically, including in the medulla oblongata and peripheral vasculature;
Alzheimer's Disease, including Mild Cognitive Impairment; and
Encephalitis due to viral (including but not limited to COVID-19, Herpes simplex, Varicella zoster, enterovirus, flavivirus) or bacterial infection, autoimmune disease, or insect bites (including Lyme's Disease).

Also provided herein is the use of a compound of Formula (I), or a compound of Table 1, or a pharmaceutically acceptable salt thereof in the manufacture of a medicament for treatment of a disease or condition mediated by NAMPT activity in a subject. In some aspects, provided is a compound or composition as described herein for use in a method of treatment of the human or animal body by therapy. In some embodiments, provided herein are compounds of Formula (I), or a compound of Table 1, or a pharmaceutically acceptable salt thereof, for use in a method of treatment of the human or animal body by therapy. In some embodiments, provided herein are compounds of Formula (I), or a compound of Table 1, or a pharmaceutically acceptable salt thereof, for use in treating a disease or condition mediated by NAMPT activity. In some embodiments, the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, or a muscle disease or muscle wasting disorder.

Also provided herein are compositions (including pharmaceutical compositions) as described herein for the use in treating, preventing, and/or delaying the onset and/or development of a disease described herein and other methods described herein. In certain embodiments, the composition comprises a pharmaceutical formulation which is present in a unit dosage form.

In some embodiments, the subject is a mammal. In some embodiments, the subject is a mouse, rat, dog, cat, rabbit, pig, sheep, horse, cow, or human. In some embodiments, the subject is a human.

There are numerous conditions in which small molecule-mediated stimulation of NAMPT activity that boosts NAD+ levels would potentially be clinically beneficial (Strømland et al., Biochem Soc Trans. 2019, 47(1):119-130; Ralto et al., Nat Rev Nephrol. 2019; Fang et al., Trends Mol Med. 2017, 23(10):899-916; Yoshino et al., Cell Metab. 2011,14(4):528-36; Yang and Sauve, Biochim Biophys Acta. 2016, 1864:1787-1800; Verdin, Science. 2015, 350(6265):1208-13). These conditions include, but are not limited to, cardiac diseases, chemotherapy induced tissue damage, renal diseases, metabolic diseases, muscular diseases, neurological diseases and injuries, diseases caused by impaired stem cell function, and DNA damage and primary mitochondrial disorders. In some embodiments, the disease or condition mediated by NAMPT activity is a cardiac disease, chemotherapy induced tissue damage, a renal disease, a metabolic disease, a muscular disease, a neurological disease or injury, a disease caused by impaired stem cell function, or DNA damage and primary mitochondrial disorder.

Cardiac diseases. In various preclinical models of heart failure NAD as well as NAMPT levels are decreased. In these models, cardiac function can be rescued, either by restoring NAD via oral supplementation or overexpression of NAMPT (Diguet et al, Circulation. 2018, 137:2256-2273; Zheng et al., Clin Sci (Lond). 2019, 133(13):1505-1521; Smyrnias et al., J Am Coll Cardiol. 2019, 73(14):1795-1806). Thus, increasing the catalytic efficiency of NAMPT with a small molecule activator to compensate for the decreased protein levels is a promising strategy to treat various forms of heart failure.

Chemotherapy induced tissue damage. Use of chemotherapy regimens frequently is limited by toxicity to healthy tissues and severe oxidative stress is thought to play a major role. NAD boosting has been shown to trigger a strong antioxidant response. Therefore, NAMPT activators are considered broadly useful in various settings of chemotherapy to prevent reversible and irreversible secondary pathologies. Examples are anthracycline and trastuzumab cardiotoxicity, cisplatin induced kidney injury, peripheral neuropathies induced by cisplatin, paclitaxel, vincristine and other agents. Neuroprotection by NAMPT activation is also useful in treating/preventing chemotherapy associated cognitive (“chemo brain”), which is caused by destruction of healthy nerve tissue, both during active treatment and long after treatment has been halted. For instance, see Zheng et al., Clin Sci (Lond). 2019, 133(13):1505-1521.

Renal diseases. Renal diseases are highly prevalent and an area of urgent unmet medical need. In approximately 3% of hospitalized patients, acute kidney injury (AKI) is diagnosed. A subset of patients will progress to chronic kidney disease that may require long-term dialysis or kidney transplantation. A key feature of kidney dysfunction is a decrease in the activities of SIRT1 and SIRT3, characterized by a reduction of the sirtuin substrate NAD, primarily due to impairment of de novo NAD+ synthesis. NAMPT is robustly expressed during kidney injury, thus small molecule activation with NAMPT is considered an effective measure to prevent AKI. Similarly, kidney mesangial cell hypertrophy exhibits depletion of NAD+, and restoration of intracellular NAD+ levels is considered efficacious. For instance, see Poyan Mehr et al., Nat Med. 2018, September; 24(9): 1351-9.

Metabolic disease. NAD+ boosting improves insulin sensitivity, dyslipidemia, mitochondrial function in metabolic disease and protects from/improves non-alcoholic and alcoholic steatohepatitis in preclinical models. More than 3 million people per year in the U.S. alone are diagnosed with non-alcoholic steatohepatitis and it is one of the leading causes of liver transplantation. See Guarino and Dufour, Metabolites. 2019 Sep. 10; 9(9), pii: E180; Yoshino et al., Cell Metab. 2011, 14(4):528-36.

Muscular diseases. Preclinical data has suggested that NAD+ boosting strategies could alleviate skeletal muscle dysfunction in a number of conditions, including Duchenne's muscular dystrophy, and age-related sarcopenia. See Zhang et al., Clin Sci (Lond). 2019, 133(13):1505-1521; Mohamed et al., Aging (Albany NY). 2014, 6(10):820-34; Ryu et al., Sci Transl Med. 2016, 8(361):361ra139.

Neurological diseases and injuries. Repletion of NAD by means of NAMPT activation is neuroprotective and of therapeutic benefit in a wide range of preclinical models of neurological diseases and injuries, including age-related cognitive decline, glaucoma, ischemic stroke, and ALS. See Johnson et al., NPJ Aging Mech Dis. 2018, 4:10; Harlan et al., J Biol Chem. 2016, 291(20):10836-46; Zhao et al., Stroke. 2015, July; 46(7):1966-74; Williams et al., Front Neurosci. 2017 Apr. 25; 11:232; Blacher et al Nature, 2019, volume 572, 474-480; Harlan et al., 2020. Experimental Neurology 327:113219.

Diseases caused by impaired stem cell function. NAD boosting promotes stem cell activation and hematopoiesis and is useful in accelerating the expansion of stem cell populations following a stem cell transplant. See Pi et al., Aging (Albany NY). 2019, 11(11):3505-3522.

DNA damage disorders and primary mitochondrial disorders. NAMPT activators will also be useful in the treatment of DNA damage disorders which are associated with an accelerated aging phenotype, such as Xeroderma pigmentosum, Cockayne syndrome, and Ataxia telangiectasia. Similarly, there are several primary mitochondrial disorders with shared symptoms and manifestations for which NAD boosting via NAMPT activation may be a suitable therapeutic intervention. See Fang et al, Cell. 2014, 157(4):882-896; Khan et al, EMBO Mol Med. 2014, June; 6(6):721-31; Cerutti et al., Cell Metab. 2014, 19(6):1042-9.

Provided in some embodiments are methods of treating a disease or condition mediated by NAMPT activity in a subject in need thereof, comprising administering to the individual or subject in need thereof a compound of Formula (I), or a compound of Table 1, or a pharmaceutically acceptable salt thereof, wherein the disease or condition is selected from the group consisting of cardiac diseases, chemotherapy induced tissue damage, renal diseases, metabolic diseases, muscular diseases, neurological diseases and injuries, diseases caused by impaired stem cell function, and DNA damage and primary mitochondrial disorders.

Additional applications of small molecule NAMPT activators are provided in Table 2.

TABLE 2

| | |
|---|---|
| Cancer and | Anthracycline and trastuzumab cardiotoxicity |
| Chemotherapy | Proteasome inhibitor cardiotoxicity |
| induced tissue | Cisplatin induced kidney injury |
| damage | Prevention/treatment of cognitive dysfunction resulting |
| | from chemotherapy (“chemo brain”) |
| | Chemotherapy induced impairment of hematopoiesis |
| | and myelosuppression |
| | Cachexia of cancer |
| | Chemoprevention of non-melanoma skin cancer in high |
| | risk patients |
| | chemoprevention of hepatocellular carcinoma |
| Cardiovascular | Heart failure with reduced ejection fraction |
| diseases | Heart failure with preserved ejection fraction |
| | Hypertrophic cardiomyopathy |
| | Cardiac arrhythmias |
| | Duchenne Muscular Dystrophy-related cardiac |
| | dysfunction |
| | Cardiac dysfunction associated with Scleroderma, |
| | Lupus, Mitochondrial Disorders, Kawasaki Disease |
| | Hypertension |
| | Myocardial Infarction |
| Renal diseases | Acute kidney injury including nephropathy following |
| | major surgeries including cardiac and vascular surgeries |
| | Acute kidney injury following hypotension, |
| | hemorrhagic shock, or cardiac arrest |
| | Acute kidney injury following exposure to contrast |
| | imaging agents used for MRI, CT scans, or other |
| | imaging modalities, particularly in the context of |
| | diabetes |
| | Chronic kidney disease |
| | Glomerular nephritis |
| | Kidney mesangial cell hypertrophy |
| | Arterial venous fistula maturation |
| Chronic | Chronic obstructive pulmonary disease |
| inflammatory | Asthma |
| and fibrotic | Scleroderma |
| diseases | Dermatomyositis |
| | Lupus erythematosus |
| | Rheumatoid arthritis and spondyloarthropathy |
| | Juvenile idiopathic arthritis |
| | Crohn's disease |
| | Inflammatory Bowel Disease |
| | Eczema |
| | Psoriasis and psoriatic arthritis |
| | Idiopathic pulmonary fibrosis |
| Vascular | Arterial and venous thrombosis |
| diseases | Ischemic Stroke |
| | Arteriosclerosis |
| Metabolic | Obesity |
| dysfunction | Diabetes |
| | Metabolic Syndrome |
| | Alcoholic steatohepatitis |
| | Non-alcoholic steatohepatitis |
| | Dyslipidemia |
| | Diabetic neuropathy |
| | Diabetic gastroparesis |
| Muscular | Muscular dystrophies, including: Duchenne, Becker's, |
| diseases | Congenital, Distal, Emery-Dreifuss', Facioscapulo- |
| | humeral, Limb-girdle, myotonic, and oculopharyngeal |
| | Sarcopenia |
| | Frailty |
| | Polymyositis |
| | Muscle stem cell senescence developed in the |
| | context of nutritional deficiencies |
| | Non-mitochondrial myopathies such as inherited |
| | myopathies, myotonia, congenital myopathies selected |
| | from nemaline myopathy, multi/minicore myopathy, |
| | centronuclear myopathy and metabolic myopathies, |
| | inflammatory myopathies |
| Neurological | Depression |
| diseases | Frontotemporal dementia |
| and injuries | Multiple sclerosis |
| | Amyotrophic lateral sclerosis |
| | Peripheral neuropathy due to diabetes, chemotherapy |
| | Alzheimer's disease, including mild cognitive |
| | impairment |
| | Parkinson's disease |
| | Huntington's Disease |
| | Spinal muscular atrophy |
| | Spinocerebellar ataxias |
| | Spastic paraplegias |
| | Glaucoma |
| | Age-related macular degeneration |
| | Age-related cognitive decline |
| | Noise induced and age-related hearing loss |
| | Ischemic stroke |
| | Traumatic brain injury |
| | Neonatal nerve damage |
| | Optic nerve injury |
| | Spinal cord injuries |
| | Peripheral neuropathies or tissue inflammation induced |
| | by cisplatin, paclitaxel, vincristine, other |
| | chemotherapeutic agents, or radiation. |
| | Peripheral neuropathies (length and non-length |
| | dependent) affecting motor, sensory, or autonomic |
| | nerves, arising from: diabetes, impaired glucose |
| | tolerance, hypertension, infection, trauma, autoimmune |
| | disorders, vasculitis, arteriosclerosis, vitamin |
| | deficiencies (particularly B6 and B12), alcoholism, liver |
| | or kidney disease, or exposure to toxins |
| DNA damage | Xeroderma pigmentosum |
| disorders | Cockayne syndrome |
| and Primary | Ataxia telangiectasia |
| Mitochondrial | MEGDEL syndrome |
| Disorders | Charcot-Marie-Tooth type 2 |
| | Primary Mitochondrial Diseases (Disorders) including |
| | NARP, MELAS, Chronic Progressive External |
| | Ophthalmoplegia, Leigh's disease, Leber's Hereditary |
| | Optic Neuropathy, MERRF, Barth Syndrome, Luft |
| | Disease, Kearns Sayre Syndrome, Autosomal dominant |
| | optic atrophy |
| | Friedreich's ataxia |
| | Werner syndrome |
| General | Tissue repair following physical trauma, hemorrhagic |
| | shock, tissue grafting, organ transplant including heart, |
| | lung, liver, and kidney |
| | Stem cell therapies, including hematopoietic stem cell |
| | transfer, allogenic mesenchymal stem therapy for acute |
| | graft-vs-host disease, limbal stem cell deficiency due to |
| | genetic or acquired conditions that compromise normal |
| | turnover of the corneal epithelium |

In some embodiments, the disease or condition mediated by NAMPT activity is cancer and chemotherapy-induced tissue damage, a cardiovascular disease, a renal disease, chronic inflammatory and fibrotic disease, a vascular disease, metabolic dysfunction, a muscular disease, a neurological disease or injury, or a DNA damage disorder or primary mitochondrial disorder. Provided in some embodiments are methods of treating a disease or condition mediated by NAMPT activity in a subject in need thereof, comprising administering to the individual or subject in need thereof a compound of Formula (I), or a compound of Table 1, or a pharmaceutically acceptable salt thereof. In some embodiments, the disease or condition is cancer or chemotherapy induced tissue damage, a cardiovascular disease, a renal disease, a chronic inflammatory or fibrotic disease, a vascular disease, metabolic dysfunction, a muscular disease, a neurological disease or injury, a DNA damage disorder or Primary Mitochondrial Disorder, including any of the diseases listed in Table 2.

### Dosages

The compounds and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the compound pharmacokinetics.

A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The compounds disclosed and/or described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pennsylvania.

In some embodiments, the compositions will take the form of a pill or tablet and thus the composition may contain, along with a compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include a compound disclosed and/or described herein and one or more additional medicinal agents, pharmaceutical agents, adjuvants, and the like. Suitable medicinal and pharmaceutical agents include those described herein.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of a heart disease in an individual or subject in need thereof. A kit may additionally contain any materials or equipment that may be used in the administration of the compound or composition, such as vials, syringes, or IV bags. A kit may also contain sterile packaging.

### Combinations

The compounds and compositions described and/or disclosed herein may be administered alone or in combination with other therapies and/or therapeutic agents useful in the treatment of the aforementioned disorders, diseases, or conditions.

### ENUMERATED EMBODIMENTS

The following enumerated embodiments are representative of some aspects of the invention.
Enumerated Embodiment 1. A compound of Formula (I)

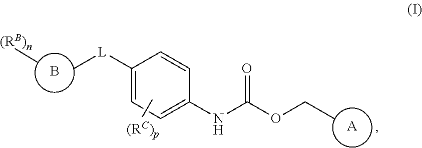

or a pharmaceutically acceptable salt thereof, wherein:
Ring A is: i) 5- to 6-membered heteroaryl optionally substituted with 1 to 4 R^{A}, or

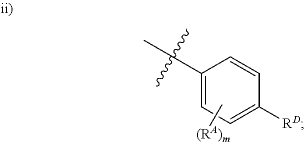

each R^{A} is independently selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
R^{D} is selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
L is selected from the group consisting of a bond, C₁-C₆ alkylene,
#—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$,
#—C(O)—N(R^{L})—(C₁-C₆ alkylene)-$, #—(C₁-C₆ alkylene)-C(O)—N(R^{L})—$,
#—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$, #—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl;
Ring B is 4- to 10-membered heterocycloalkyl, 3- to 8-membered cycloalkyl, 5- to 6-membered heteroaryl, or phenyl;
each R^{B} is independently selected from the group consisting of:
halogen;
—OH;
oxo;
cyano;
—O(C₁-C₆ alkyl) optionally substituted with phenyl or 1 to 3 independently selected halogens;
—C(O)(C₁-C₆ alkyl);
—C(O)O(C₁-C₆ alkyl);
phenyl;
5- to 6-membered heteroaryl;
4- to 8-membered heterocycloalkyl;
3- to 8-membered cycloalkyl;
—C(O)(3- to 8-membered cycloalkyl);
—C(O)(4-8-membered heterocycloalkyl);
—S(O)₂(C₁-C₆ alkyl);
—S(O)₂(3- to 8-membered cycloalkyl);
—C(O)NR^{B1}R^{B2};
—S(O)₂NR^{B1}R^{B2};
—NR^{C1}S(O)₂NR^{B1}R^{B2};
—(C═N—R^{C1})—NR^{B1}R^{B2};
—NR^{C1}—(C═N—R^{C1})—NR^{B1}R^{B2};
—NR^{C1}—(C═N—CN)—NR^{B1}R^{B2}; and
C₁-C₆ alkyl optionally substituted with 1 to 5 substituents independently selected from the group consisting of halogen, —OH, and —O(C₁-C₆ alkyl);
wherein R^{C1}, R^{B1} and R^{B2} are each independently hydrogen or C₁-C₆ alkyl;
m is 0, 1, 2, 3, or 4;
n is 0, 1, 2, 3, 4, or 5;
R^{C} is halogen, cyano, C₁-C₆ alkyl, OH, —O(C₁-C₆ alkyl), or 3- to 8-membered cycloalkyl; and
p is 0, 1, 2, 3, or 4;
wherein:
a) when L is a bond, ring B is selected from the group consisting of

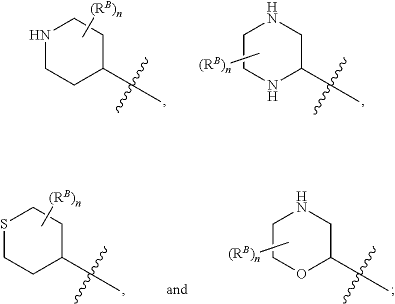

b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.
Enumerated Embodiment 2. The compound of Enumerated Embodiment 1, or a pharmaceutically acceptable salt thereof, wherein Ring A is

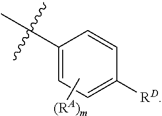

Enumerated Embodiment 3. The compound of Enumerated Embodiment 1 or 2, or a pharmaceutically acceptable salt thereof, wherein R^{D} is halogen or —O(C₁-C₆ alkyl) optionally substituted with 1 to 3 independently selected halogens.
Enumerated Embodiment 4. The compound of any one of Enumerated Embodiments 1-3, or a pharmaceutically acceptable salt thereof, wherein R^{D} is halogen.
Enumerated Embodiment 5. The compound of any one of Enumerated Embodiments 1-4, or a pharmaceutically acceptable salt thereof, wherein R^{D} is fluorine.
Enumerated Embodiment 6. The compound of any one of Enumerated Embodiments 1-4, or a pharmaceutically acceptable salt thereof, wherein R^{D} is chlorine.
Enumerated Embodiment 7. The compound of Enumerated Embodiment 1, or a pharmaceutically acceptable salt thereof, wherein Ring A is 5- to 6-membered heteroaryl optionally substituted with 1 to 4 R^{A}.
Enumerated Embodiment 8. The compound of any one of Enumerated Embodiment 1 or 7, or a pharmaceutically acceptable salt thereof, wherein Ring A is 5-membered heteroaryl optionally substituted with 1 to 4 R^{A}.
Enumerated Embodiment 9. The compound of any one of Enumerated Embodiments 1, 7, or 8, or a pharmaceutically acceptable salt thereof, wherein Ring A is oxazolyl, pyrazolyl, thiazolyl, isothiazolyl, or isoxazolyl, each of which is optionally substituted with 1 to 3 R^{A}.
Enumerated Embodiment 10. The compound of any one of Enumerated Embodiments 1, or 7-9, or a pharmaceutically acceptable salt thereof, wherein Ring A is oxazolyl optionally substituted with 1 to 2 R^{A}.
Enumerated Embodiment 11. The compound of any one of Enumerated Embodiments 1, or 7-10, or a pharmaceutically acceptable salt thereof, wherein Ring A is oxazol-5-yl optionally substituted with 1 to 2 R^{A}.
Enumerated Embodiment 12. The compound of Enumerated Embodiment 1 or 7, or a pharmaceutically acceptable salt thereof, wherein Ring A is 6-membered heteroaryl optionally substituted with 1 to 4 R^{A}.
Enumerated Embodiment 13. The compound of any one of Enumerated Embodiments 1, 7, or 12, or a pharmaceutically acceptable salt thereof, wherein Ring A is pyridinyl, pyridazinyl, or pyrimidinyl, each of which is optionally substituted with 1 to 4 R^{A}.
Enumerated Embodiment 14. The compound of any one of Enumerated Embodiments 1, 7, 12, or 13, or a pharmaceutically acceptable salt thereof, wherein Ring A is pyridinyl optionally substituted with 1 to 4 R^{A}.
Enumerated Embodiment 15. The compound of any one of Enumerated Embodiments 1, 7, or 12-14, or a pharmaceutically acceptable salt thereof, wherein Ring A is pyridin-4-yl optionally substituted with 1 to 4 R^{A}.
Enumerated Embodiment 16. The compound of any one of Enumerated Embodiments 1-15, wherein each R^{A} is independently selected from the group consisting of halogen and C₁-C₆ alkyl.
Enumerated Embodiment 17. The compound of any one of Enumerated Embodiments 1-16, or a pharmaceutically acceptable salt thereof, wherein Ring B is 4- to 8-membered heterocycloalkyl, 3- to 8-membered cycloalkyl, or 5- to 6-membered heteroaryl.
Enumerated Embodiment 18. The compound of any one of Enumerated Embodiments 1-17, or a pharmaceutically acceptable salt thereof, wherein Ring B is pyridinyl, piperazinyl, morpholinyl, piperidinyl, pyrrolidinyl, oxazolyl, or pyrazolyl.
Enumerated Embodiment 19. The compound of any one of Enumerated Embodiments 1-18, or a pharmaceutically acceptable salt thereof, wherein Ring B is pyridinyl, piperazinyl, morpholinyl, piperidinyl, or pyrrolidinyl.
Enumerated Embodiment 20. The compound of any one of Enumerated Embodiments 1-19, or a pharmaceutically acceptable salt thereof, wherein Ring B is

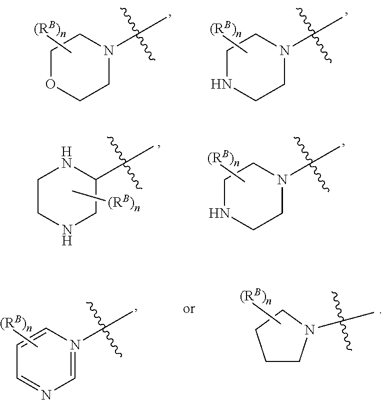

Enumerated Embodiment 21. The compound of any one of Enumerated Embodiments 1-20, or a pharmaceutically acceptable salt thereof, wherein each R^{B} is independently selected from the group consisting of:
halogen;
oxo;
—O(C₁-C₆ alkyl) optionally substituted with phenyl or 1 to 3 independently selected halogens;
C₁-C₆ alkyl optionally substituted with 1 to 5 substituents independently selected from halogen, —OH, and —O(C₁-C₆ alkyl);
—S(O)₂(C₁-C₆ alkyl);
—S(O)₂NR^{B1}R^{B2};
—C(O)NR^{B1}R^{B2};
—C(O)(C₁-C₆ alkyl);
4- to 8-membered heterocycloalkyl; and
5- to 6-membered heteroaryl.
Enumerated Embodiment 22. The compound of any one of Enumerated Embodiments 1-21, or a pharmaceutically acceptable salt thereof, wherein each R^{B} is independently selected from the group consisting of oxo, —C(O)(C₁-C₆ alkyl), —C(O)NR^{B1}R^{B2}, —S(O)₂(C₁-C₆ alkyl), —S(O)₂NR^{B1}R^{B2}, unsubstituted C₁-C₆ alkyl, 4- to 8-membered heterocycloalkyl, and 5- to 6-membered heteroaryl.
Enumerated Embodiment 23. The compound of any one of Enumerated Embodiments 1-22, or a pharmaceutically acceptable salt thereof, wherein each R^{B} is independently selected from the group consisting of oxo, —C(O)(C₁-C₃ alkyl), —C(O)N(Me)₂, —S(O)₂Me, —S(O)₂N(Me)₂, methyl, oxetanyl, and pyridinyl.
Enumerated Embodiment 24. The compound of any one of Enumerated Embodiments 1-23, or a pharmaceutically acceptable salt thereof, wherein L is a bond, C₁-C₆ alkylene, #—C(O)—N(R^{L})—(C₁-C₆ alkylene)-$, #—C(O)—(C₁-C₆ alkylene)-$, or #—(C₁-C₆ alkylene)-C(O)—N(R^{L})—(C₁-C₆ alkylene)-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl.
Enumerated Embodiment 25. The compound of any one of Enumerated Embodiments 1-24, or a pharmaceutically acceptable salt thereof, wherein each C₁-C₆ alkylene of L is substituted with 1 to 3 substituents independently selected from the group consisting of halogen, —OH, and C₁-C₆ alkyl.
Enumerated Embodiment 26. The compound of any one of Enumerated Embodiments 1-25, or a pharmaceutically acceptable salt thereof, wherein each C₁-C₆ alkylene of L is substituted with 1 to 3 substituents independently selected from the group consisting of halogen and C₁-C₆ alkyl.
Enumerated Embodiment 27. The compound of any one of Enumerated Embodiments 1-26, or a pharmaceutically acceptable salt thereof, wherein each C₁-C₆ alkylene of L is substituted with 1 to 3 substituents independently selected from the group consisting of fluoro and methyl.
Enumerated Embodiment 28. The compound of any one of Enumerated Embodiments 1-24, or a pharmaceutically acceptable salt thereof, wherein each C₁-C₆ alkylene of L is unsubstituted.
Enumerated Embodiment 29. The compound of any one of Enumerated Embodiments 1-24, or a pharmaceutically acceptable salt thereof, wherein L is a bond, C₁-C₃ alkylene, or #—C(O)—N(R^{L})—(C₁-C₃ alkylene)-$, wherein #represents the attachment point to ring B, and $ represents the attachment point to the remainder of the molecule; wherein each C₁-C₆ alkylene of L is optionally substituted with 1 to 3 substituents independently selected from the group consisting of halogen, —OH, and C₁-C₆ alkyl; and wherein each R^{L} is independently hydrogen or C₁-C₆ alkyl.
Enumerated Embodiment 30. The compound of any one of Enumerated Embodiments 1-24 or 29, or a pharmaceutically acceptable salt thereof, wherein L is a bond, —CH₂—, —CH₂CH₂—, or

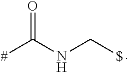

Enumerated Embodiment 31. The compound of any one of Enumerated Embodiments 1-6 or 16-30, or a pharmaceutically acceptable salt thereof, wherein m is 1.
Enumerated Embodiment 32. The compound of any one of Enumerated Embodiments 1-6 or 17-30, or a pharmaceutically acceptable salt thereof, wherein m is 0.
Enumerated Embodiment 33. The compound of any one of Enumerated Embodiments 1-32, or a pharmaceutically acceptable salt thereof, wherein n is 0, 1, or 2.
Enumerated Embodiment 34. The compound of any one of Enumerated Embodiments 1-33, or a pharmaceutically acceptable salt thereof, wherein R^{C} is fluoro.
Enumerated Embodiment 35. The compound of any one of Enumerated Embodiments 1-34, or a pharmaceutically acceptable salt thereof, wherein p is 0.
Enumerated Embodiment 36. A compound selected from the group consisting of compounds of Table 1, or a pharmaceutically acceptable salt thereof.
Enumerated Embodiment 37. A pharmaceutical composition comprising a compound according to any one of Enumerated Embodiments 1-36, or a pharmaceutically acceptable salt thereof, and a pharmaceutically acceptable excipient.
Enumerated Embodiment 38. A method of treating a disease or condition mediated by NAMPT activity in a subject in need thereof, comprising administering to the subject a compound of any one of Enumerated Embodiments 1-36, or a pharmaceutically acceptable salt thereof, or a pharmaceutical composition of Enumerated Embodiment 37.
Enumerated Embodiment 39. The method of Enumerated Embodiment 38, wherein the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, and a muscle disease or muscle wasting disorder.
Enumerated Embodiment 40. The method of Enumerated Embodiment 38, wherein the disease or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

### General Synthetic Methods

Compounds of Formula (I), or any variation or embodiment thereof, or a salt of any of the foregoing, will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate. Unless otherwise specified, the variables are as defined above in reference to Formula (I).

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

General methods of preparing compounds described herein are depicted in exemplified methods below. Variable groups in the schemes provided herein are defined as for Formula (I), or any variation thereof. Other compounds described herein may be prepared by similar methods.

In some embodiments, compounds provided herein may be synthesized according to Schemes A1, A2, A3 or A4, wherein Ring A, Ring B, L, R^{B}, R^{C}, n, and p are as defined for formula (I) or any variation thereof detailed herein.

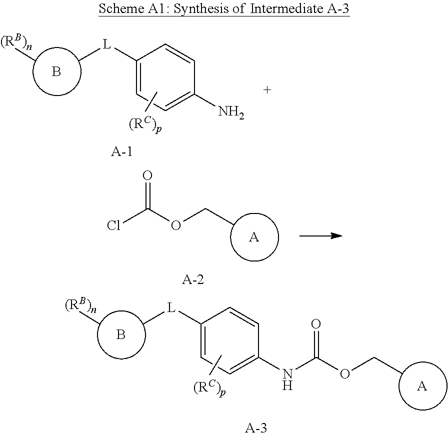

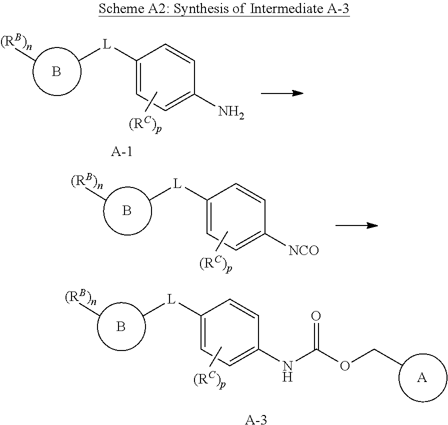

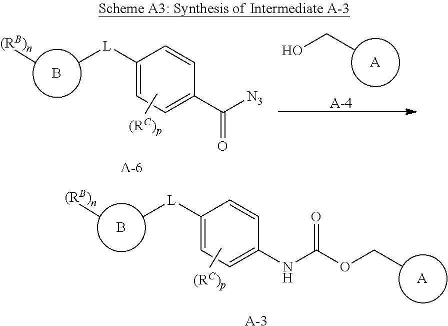

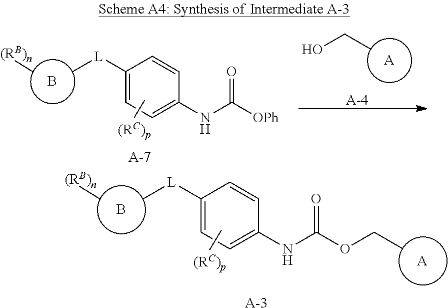

In certain embodiments compounds provided herein may be synthesized according to Scheme Ala, A2a, A3a or A4a, wherein Ring A, Ring B, L, R^{B}, R^{C}, n, and p are as defined for formula (I) or any variation thereof detailed herein.

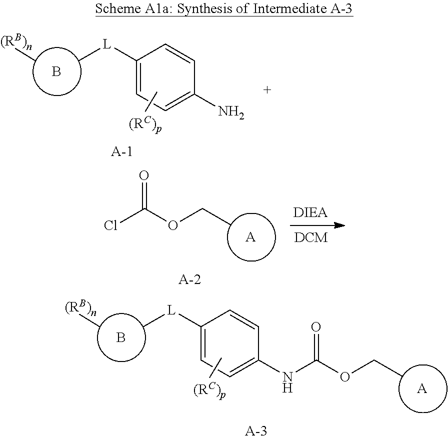

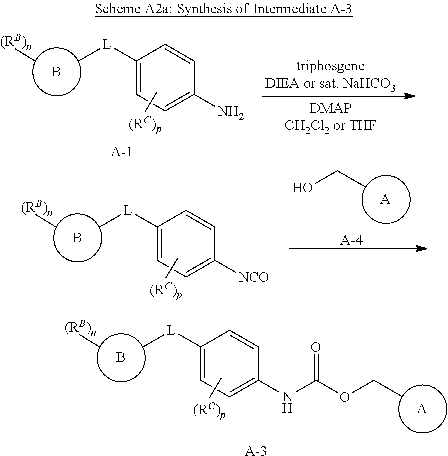

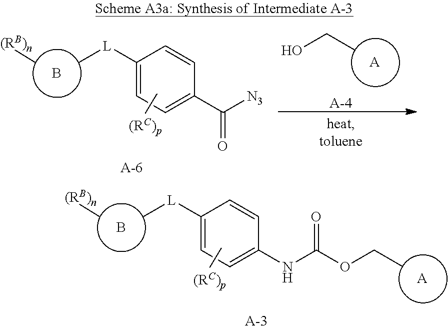

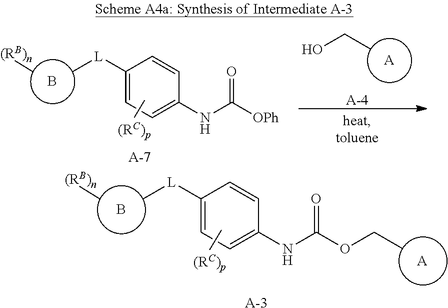

Particular non-limiting examples are provided in the Example section below.

### EXAMPLES

The following examples are offered to illustrate but not to limit the compositions, uses, and methods provided herein. The compounds are prepared using the general methods described above or below. Starting materials were either purchased from commercial source or prepared according to literature procedures.

The following abbreviations are used throughout the Examples: TEA (triethylamine), DCM (dichloromethane), (Boc)₂O (di-tert-butyl decarbonate), EA (Ethyl acetate), PE (Petroleum ether, DMF (N,N-dimethylformamide), DIEA (N-ethyl-N-isopropylpropan-2-amine), DMAP [4-(dimethylamino)pyridine], HATU (1-[Bis(dimethylamino)methylene]-1H-1,2,3-triazolo[4,5-b]pyridinium 3-oxid hexafluorophosphate), HOAt (1-Hydroxy-7-azabenzotriazole), HOBt (Hydroxybenzotriazole), EDCI (1-Ethyl-3-(3-dimethylaminopropyl)carbodiimide), MeOH (methanol), EtOH (ethanol), iPrOH (propan-2-ol), ACN (acetonitrile), TFA (trifluoroacetic acid), DPPA (Diphenylphosphoryl azide), DBU (1,8-Diazabicyclo(5.4.0)undec-7-ene), THF (tetrahydrofuran), PPh₃ (triphenylphosphine), SM (starting material), Hex (hexane), NCS (N-chlorosuccinimide), r.t. or rt (room temperature around 21 to 24° C.), DCE (dichloroethane), FA (formic acid), CHCl₃ (chloroform), BnBr (benzyl bromide), HCl (hydrogen chloride), equiv (equivalent), and DSC (bis(2,5-dioxopyrrolidin-1-yl) carbonate), HBTU (O-(benzotriazol-1-yl)-N,N,N′,N′-tetramethyluronium hexafluorophosphate).

### Aniline Syntheses

### Example A

### Synthesis of Aniline Intermediates 1.1, 1.2, and 1.3

### Step 1: Synthesis of tert-butyl (S)-(4-(1-cyclobutyl-5-oxopiperazin-2-yl)phenyl)carbamate

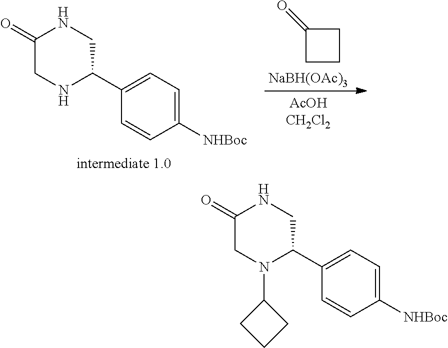 [SMILES: CC(C)(C)OC(=O)Nc1ccc([C@H]2CNC(=O)CN2)cc1 ; O=C1CCC1 ; CC(C)(C)OC(=O)Nc1ccc([C@H]2CNC(=O)CN2C2CCC2)cc1 ; "NaBH(OAc)3"]

Ketone (252 mg, 3.6 mmol) and acetic acid (108 mg, 1.8 mmol) were added to a stirring solution of tert-butyl (S)-(4-(5-oxopiperazin-2-yl)phenyl)carbamate (Ref. 1) (252 mg, 1.8 mmol) in CH₂Cl₂ (5 mL) at r.t. and the reaction stirred for 30 min. Sodium triacetoxyborohydride (763 mg, 3.6 mmol) was added and the reaction was stirred for 2 h before being confirmed complete by LCMS. The reaction was then quenched with saturated sodium bicarbonate (15 mL), extracted with CH₂Cl₂ (3×15 mL), organic solutions were combined, dried over sodium sulfate, filtered, and solvent removed by rotary evaporation. The crude material was carried forward without additional purification. LRMS (ES) m/z: 346.1 [M+H]⁺.

### Step 2: Synthesis of (S)-5-(4-aminophenyl)-4-cyclobutylpiperazin-2-one

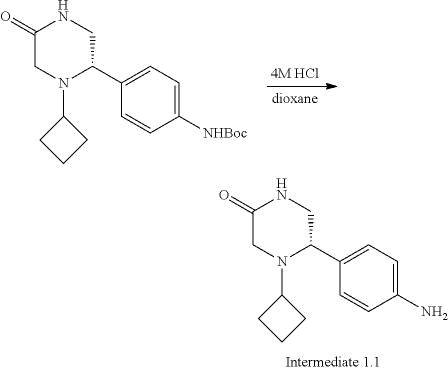 [SMILES: CC(C)(C)OC(=O)Nc1ccc([C@H]2CNC(=O)CN2C2CCC2)cc1 ; Nc1ccc([C@H]2CNC(=O)CN2C2CCC2)cc1]

HCl (4M in dioxanes, 2.5 mL, 10 mmol) was added to a stirring solution of tert-butyl (S)-(4-(1-methyl-5-oxopiperazin-2-yl)phenyl)carbamate (1.8 mmol) at r.t. After 6 h, the reaction was concentrated by rotary evaporation, triturated with EtOAc, filtered, and the resultant solid dried under high vacuum to give the desired product as an off-white solid (400 mg, 70% over 2 steps). LCMS-APCI (POS.) m/z: 246.1 [M+H]⁺.

Compounds in the following table were prepared in a similar manner as intermediate 1.1, using the intermediates and reagents as listed.

| Intermediate | Reagents | Structure, Name and Data |
|---|---|---|
| 1.2 | oxetan-3-one | 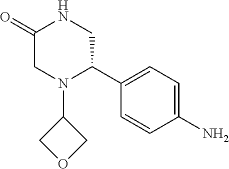 [SMILES: Nc1ccc([C@H]2CNC(=O)CN2C2COC2)cc1] |
| | | Intermediate 1.2: (S)-5-(4-aminophenyl)-4- |
| | | (oxetan-3-yl)piperazin-2-one. LCMS-ESI |
| | | (POS.) m/z: 248.1 [M + H]⁺. |
| 1.3 | acetone | 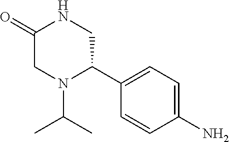 [SMILES: CC(C)N1CC(=O)NC[C@@H]1c1ccc(N)cc1] |
| | | Intermediate 1.3: (S)-5-(4-aminophenyl)-4- |
| | | isopropylpiperazin-2-one. LCMS-APCI |
| | | (POS.) m/z: 234.1 [M + H]⁺. |
| 1.4 | fomaldehyde | 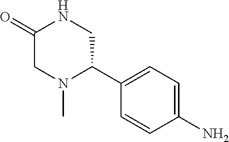 |
| | | Intermediate 1.4: (S)-5-(4-aminophenyl)-4- |
| | | methylpiperazin-2-one. LCMS-ESI (POS.) |
| | | m/z: 206.1 [M + H]⁺. |

### Example B

### Synthesis of Aniline Intermediate 2.1

### Step 1: Synthesis of tert-butyl (E)-4-(4-nitrostyryl)piperidine-1-carboxylate

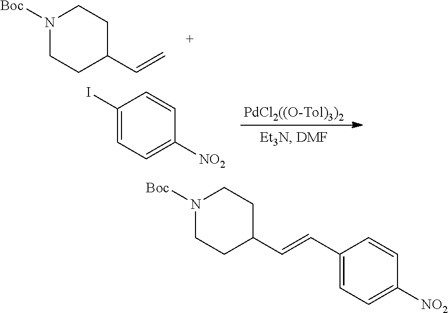

To a solution of tert-butyl 4-vinylpiperidine-1-carboxylate (5.71 g, 27 mmol, 1.80 equiv), 1-iodo-4-nitrobenzene (3.74 g, 15 mmol, 1.00 equiv), triethylamine (4.55 g, 45 mmol, 3.00 equiv) in DMF (50 mL) was added bis[tri(o-tolyl)phosphine]palladium(II) chloride (0.59 g, 0.75 mmol, 0.05 equiv). The reaction mixture was degassed for 10 min. Then it was heated at 130° C. for 24 h. The reaction mixture was extracted with ether/EtOAc (50 mL/50 mL) and H₂O (80 mL). The organic layer was concentrated and the residue was purified on silica gel with 35% EtOAc/Hex to give the desired product tert-butyl (E)-4-(4-nitrostyryl)piperidine-1-carboxylate as a yellow-reddish solid (1.70 g, 34%). LRMS (ES) m/z: 333 [M+H]⁺.

### Step 2: Synthesis of tert-butyl 4-(4-aminophenethyl)piperidine-1-carboxylate

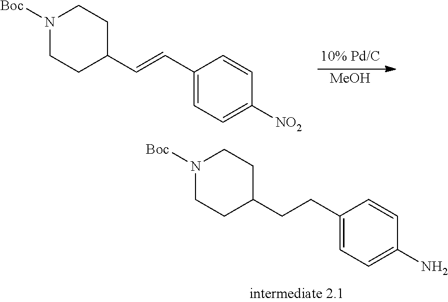

To a solution tert-butyl (E)-4-(4-nitrostyryl)piperidine-1-carboxylate (0.83 g, 2.50 mmol) in MeOH (30 mL) was added 10% Pd/C (0.27 g). The reaction mixture was stirred under H₂ at 60 psi for 30 min. The catalyst was filtered off and the filtrate was concentrated to give the desired product tert-butyl 4-(4-aminophenethyl)piperidine-1-carboxylate as a yellowish solid (0.70 g, 92%). LRMS (ES) m/z: 305 [M+H]⁺.

### Example C

### Synthesis of Aniline Intermediate 3.1

### Step 1: Synthesis of tert-butyl (4-acetylphenyl)carbamate

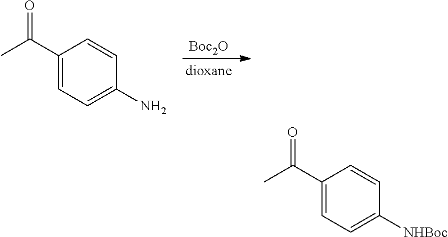

To a solution of 1-(4-aminophenyl)ethanone (2.70 g, 20 mmol, 1.00 equiv) in dry dioxane (14 mL) was added Boc₂O (5.24 g, 24 mmol, 1.20 equiv). The resulting mixture was heated at 100° C. for 5 h. The solvent was evaporated, and the residue was taken up in EtOAc (100 mL). The organic layer was washed with 1 M HCl (30 mL) three times and then brine (50 mL), dried over Na₂SO₄, and evaporated to afford the desired product tert-butyl (4acetylphenyl)carbamate as a white solid (3.60 g, 77%). LRMS (ES) m/z: 236 [M+H]⁺.

### Step 2: Synthesis of tert-butyl (4-(1-hydroxy-1-(pyridin-4-yl)ethyl)phenyl)carbamate

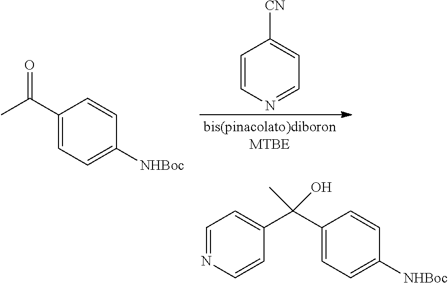 [SMILES: CC(=O)c1ccc(NC(=O)OC(C)(C)C)cc1 ; N#Cc1ccncc1 ; CC(C)(C)OC(=O)Nc1ccc(C(C)(O)c2ccncc2)cc1]

To a solution of tert-butyl (4acetylphenyl)carbamate (3.53 g, 15 mmol, 2.00 equiv) in MTBE (30 mL) was added 4-cyanopyridine (0.78 g, 7.50 mmol, 1.00 equiv) and bis-(pinacolato)diboron (4.57 g, 18 mmol, 2.40 equivalents). The resulting mixture was heated at 90° C. for 16 h. The reaction was cooled down to rt and quenched with Na₂CO₃ solution (2 M, 50 mL) and stirred under air for 15 minutes. Brine (30 mL) was added. Then the reaction mixture was extracted with EtOAc (3×50 mL). The organic layer was dried over Na₂SO₄, concentrated, and purified on silica gel to afford the desired product tert-butyl (4-(1-hydroxy-1-(pyridin-4-yl)ethyl)phenyl)carbamate as a white solid (0.70 g, 30%). LRMS (ES) m/z: 315 [[M+H]⁺.

### Step 3: Synthesis of 1-(4-aminophenyl)-1-(pyridin-4-yl)ethan-1-ol

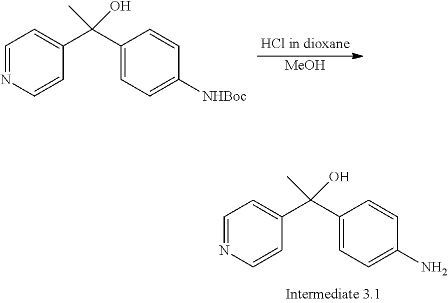 [SMILES: "MeOH" ; CC(C)(C)OC(=O)Nc1ccc(C(C)(O)c2ccncc2)cc1 ; CC(O)(c1ccncc1)c1ccc(N)cc1]

To a solution of tert-butyl (4-(1-hydroxy-1-(pyridin-4-yl)ethyl)phenyl)carbamate (0.69 g, 2.20 mmol, 1.00 equiv) in MeOH (1 mL) was added 4N HCl/dioxane (5.50 mL, 22 mmol, 10 equiv) at 0° C. The reaction mixture was stirred at rt for 16 h. The solvents were removed and the residue was used directly in the next step. LRMS (ES) m/z: 215 [M+H]⁺.

### Example D

### Synthesis of Aniline Intermediates 4.1, 4.2, 4.3 and 4.4

### Step 1: Preparation of tert-butyl 4-[(4-nitrophenyl)methoxy]piperidine-1-carboxylate

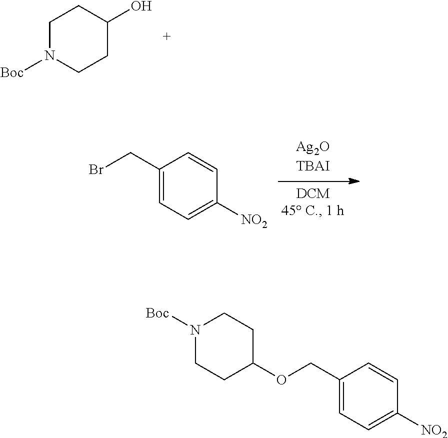

To a solution of silver (I) oxide (4.61 g, 19.87 mmol, 2.0 eq) and TBAI (0.37 g, 0.99 mmol, 0.1 eq) in DCM (10 mL) was added tert-butyl 4-hydroxypiperidine-1-carboxylate (2.0 g, 9.94 mmol, 1.0 eq). Nitrobenzyl bromide (4.29 g, 19.87 mmol, 2.0 eq) was added and the reaction was stirred at 45° C. for 1 h while shielded from light. The crude mixture was filtered over Celite, washed with DCM, and concentrated under reduced pressure. The residue brown oil was used directly in the next step without purification. Assume quantitative yield (3.34 g, 9.94 mmol, quantitative yield). LCMS-APCI (POS.) m/z: 237.1 [M+H-Boc]⁺. 1H NMR (400 MHz, DMSO-d6) δ 8.25 (dd, J=8.9, 2.1 Hz, 2H), 7.68 (d, J=8.7 Hz, 2H), 4.77 (s, 2H), 3.65 (ddt, J=13.2, 8.6, 4.2 Hz, 4H), 2.95 (s, 2H), 1.68 (dt, J=13.1, 4.0 Hz, 2H), 1.58 (s, 1H), 1.39 (s, 9H).

### Step 2: Preparation of 4-[(4-aminophenyl)methoxy]piperidin-1-yl 2,2-dimethylpropanoate

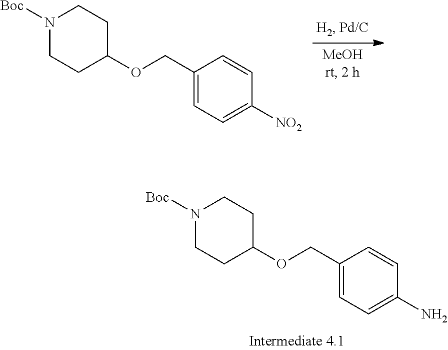 [SMILES: CC(C)(C)OC(=O)N1CCC(OCc2ccc([N+](=O)[O-])cc2)CC1 ; CC(C)(C)OC(=O)N1CCC(OCc2ccc(N)cc2)CC1]

Tert-butyl 4-[(4-nitrophenyl)methoxy]piperidine-1-carboxylate (3.34 g, 9.94 mmol, 1.0 eq) and palladium on carbon (1.06 g, 0.99 mmol, 0.2 eq) were suspended in methanol (10 mL) and stirred under hydrogen (50 psi) at r.t. for 1 h. The reaction mixture was filtered through Celite, concentrated, and purified with flash chromatography over silica gel (0-20% MeOH:DCM w/0.1% triethylamine). The product was isolated as a brown oil (1.357 g, 4.429 mmol, 44.6% yield over two steps). LCMS-APCI (POS.) m/z: 207.1 (M+H-Boc)⁺. 1H NMR (400 MHz, Methanol-d4) δ 7.09-7.05 (m, 2H), 6.72-6.67 (m, 2H), 6.64 (d, J=8.1 Hz, 1H), 4.35 (s, 2H), 3.82 (dt, J=13.6, 4.8 Hz, 3H), 3.75 (dq, J=8.6, 4.3 Hz, 2H), 3.03 (s, 3H), 1.81 (dt, J=10.3, 3.0 Hz, 3H), 1.45 (s, 3H), 1.38 (ddd, J=13.2, 8.9, 3.9 Hz, 3H).

Compounds in the following table were prepared in a similar manner as intermediate 4.1, using the starting material as listed.

| | Starting | |
|---|---|---|
| Intermediate | Material | Structure, Name and Data |
| 4.2 | tert-butyl 3- hydroxyaz- etidine-1- carboxylate | 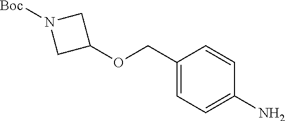 [SMILES: CC(C)(C)OC(=O)N1CC(OCc2ccc(N)cc2)C1] |
| | | tert-butyl 3-((4-aminobenzyl)oxy)azetidine-1-carboxylate. ). |
| | | LCMS-APCI (POS.) m/z: 179.1 [M + H − Boc]⁺. 1H NMR (400 |
| | | MHz, Methanol-d4) δ 7.08 (d, J = 7.9 Hz, 2H), 6.70 (d, J = 7.9 |
| | | Hz, 2H), 4.34 (s, 2H), 4.31 (s, 1H), 4.01 (t, J = 8.0 Hz, 2H), 3.70 |
| | | (dd, J = 9.6, 4.1 Hz, 2H), 1.42 (s, 9H). |
| 4.3 | 1-(oxetan- 3- yl)piperidin- 4-ol | 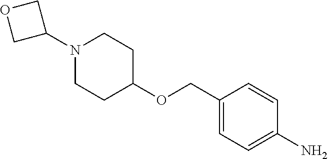 [SMILES: Nc1ccc(COC2CCN(C3COC3)CC2)cc1] |
| | | 4-({[1-(oxetan-3-yl)piperidin-4-yl]oxy}methyl)aniline. |
| | | LCMS-APCI (POS.) m/z: 263.1 [M + H − Boc]⁺. 1H NMR (400 |
| | | MHz, Methanol-d4) δ 7.10 (d, J = 8.0 Hz, 2H), 6.71 (d, J = 7.9 |
| | | Hz, 2H), 4.78 (t, J = 7.5 Hz, 3H), 4.71 (d, J = 6.8 Hz, 2H), 4.42 |
| | | (s, 2H), 4.04 (d, J = 8.9 Hz, 1H), 3.66 (s, 1H), 2.98 (t, J = 10.5 |
| | | Hz, 2H), 2.73 (s, 2H), 1.97 (d, J = 10.4 Hz, 3H), 1.87 (s, 2H). |
| 4.4 | 1-(4- hydroxy- piperidin-1- yl)ethan-1- one | 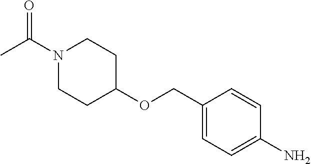 [SMILES: CC(=O)N1CCC(OCc2ccc(N)cc2)CC1] |
| | | 1-(4-((4-aminobenzyl)oxy)piperidin-1-yl)ethan-1-one. |
| | | 1H NMR (400 MHz, Methanol-d4) δ 7.09 (d, J = 7.8 Hz, 2H), |
| | | 6.70 (d, J = 7.9 Hz, 2H), 4.42 (s, 2H), 3.89 (dt, J = 12.2, 4.6 Hz, |
| | | 1H), 3.76-3.62 (m, 2H), 3.25 (t, J = 8.8 Hz, 1H), 2.08 (s, 3H), |
| | | 1.95-1.78 (m, 3H), 1.64-1.37 (m, 3H). |

### Example E

### Synthesis of Intermediates 5.1 to 5.5

### Step 1: Synthesis of (R)-1-(4-nitrobenzyl)-5-(pyridin-3-yl)pyrrolidin-2-one

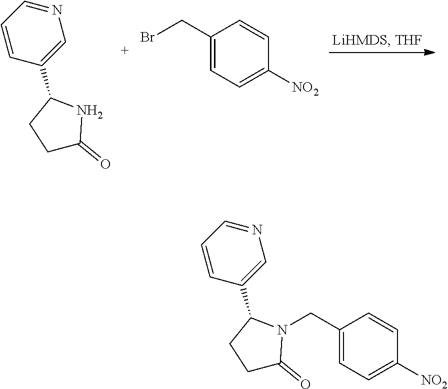

To a suspension of (5R)-5-(pyridin-3-yl)pyrrolidin-2-one (80 mg, 0.493 mmol) in THF (1.5 mL) was added lithium bis(trimethylsilyl)amide (83 mg, 0.49 mmol) at 0° C., then stirred at room temperature for 1 h. The mixture was cooled to 0° C., and the solution of 4-nitrobenzyl bromide (109 mg, 0.503 mmol) in THF (1 mL) was added at 0° C. The mixture was allowed to warm to room temperature and stirred for 4.5 h. The mixture was filtered and purified by reverse phase HPLC (0-50% MeCN/H₂O, 0.1% HCOOH) to give (5R)-1-[(4-nitrophenyl)methyl]-5-(pyridin-3-yl)pyrrolidin-2-one (22 mg, 15% yield). LCMS-APCI (POS.) m/z: 298.1 [M+H]⁺.

### Step 2: Synthesis of (R)-1-(4-aminobenzyl)-5-(pyridin-3-yl)pyrrolidin-2-one

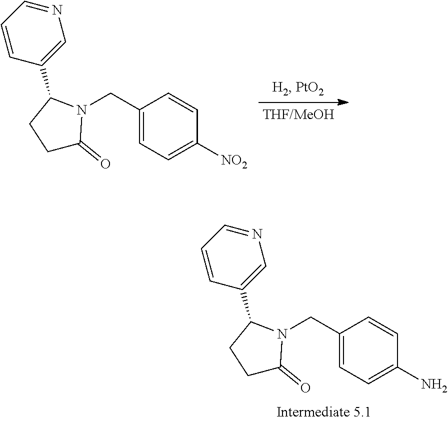 [SMILES: O=C1CC[C@H](c2cccnc2)N1Cc1ccc([N+](=O)[O-])cc1 ; Nc1ccc(CN2C(=O)CC[C@@H]2c2cccnc2)cc1]

The mixture of (5R)-1-[(4-nitrophenyl)methyl]-5-(pyridin-3-yl)pyrrolidin-2-one (22 mg, 0.078 mmol) and platinum oxide (5 mg) in THF/MeOH (2 mL:1 mL) was stirred at r.t. under hydrogen (50 psi) for 3 h. The mixture was concentrated to give (5R)-1-[(4-nitrophenyl)methyl]-5-(pyridin-3-yl)pyrrolidin-2-one (15 mg), which was used for the next step without further purification. LCMS-APCI (POS.) m/z: 268.1 [M+H]⁺.

Intermediates in the following table were prepared in a similar manner as intermediate 5.1, using the starting material and reagents as listed.

| | Starting | |
|---|---|---|
| Intermediate | Material | Structure, Name and Data |
| 5.2 | 2- azabicyclo [3.1.0] hexan-3-one | 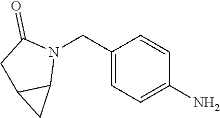 [SMILES: Nc1ccc(CN2C(=O)CC3CC32)cc1] |
| | | Intermediate 5.2: 2-(4-aminobenzyl)-2- |
| | | azabicyclo[3.1.0]hexan-3-one. LCMS-ESI |
| | | (POS.) m/z: 203.1 [M + H]⁺. |
| 5.3 | 5- azaspiro[2.4] heptan- 4-one | 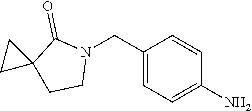 [SMILES: Nc1ccc(CN2CCC3(CC3)C2=O)cc1] |
| | | Intermediate 5.3: 5-(4-aminobenzyl)-5- |
| | | azaspiro[2.4]heptan-4-one. LCMS-ESI |
| | | (POS.) m/z: 217.1 [M + H]⁺. |
| 5.4 | 3,3- dimethyl- pyrrolidin-2- one | 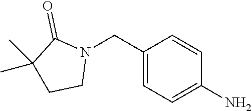 [SMILES: CC1(C)CCN(Cc2ccc(N)cc2)C1=O] |
| | | Intermediate 5.4: 1-(4-aminobenzyl)-3,3- |
| | | dimethylpyrrolidin-2-one. LCMS-ESI |
| | | (POS.) m/z: 219.1 [M + H]⁺. |
| 5.5 | 3- azabicyclo [3.1.0] hexan-2-one | 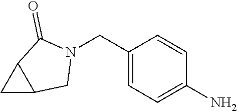 [SMILES: Nc1ccc(CN2CC3CC3C2=O)cc1] |
| | | Intermediate 5.5: 3-(4-aminobenzyl)-3- |
| | | azabicyclo[3.1.0]hexan-2-one. LCMS-ES |
| | | (Pos) m/z:203.1 [M + H]⁺. |

### Example R

### Synthesis of Intermediates 27.1 to 27.3

### Step 1: Synthesis of tert-butyl 6-(((trifluoromethyl)sulfonyl)oxy)-2-azaspiro[3.3]hept-5-ene-2-carboxylate

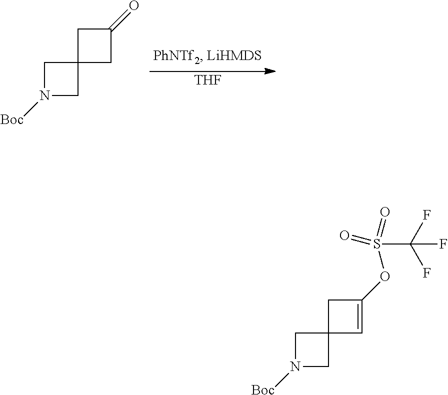 [SMILES: CC(C)(C)OC(=O)N1CC2(CC(=O)C2)C1 ; CC(C)(C)OC(=O)N1CC2(C=C(OS(=O)(=O)C(F)(F)F)C2)C1 ; "THF"]

To a solution of tert-butyl 6-oxo-2-azaspiro[3.3]heptane-2-carboxylate (5 g, 1 equiv) in THF (200 mL, 0.118 M) at −78° C. was added lithium bis(trimethylsilyl)amide (71 mL, 1 M, 3 equiv) dropwise, the mixture was stirred at −78° C. for 1 h, followed by addition of phenyl triflimide (16.9 g, 2 equiv) in THF (100 mL) dropwise. The mixture was stirred for 3 h and quenched with water. The organic portion was concentrated, diluted with water, and extracted with ethyl acetate. The combined organic layers were dried over sodium sulfate, concentrated, and purified by silica gel chromatography (0-20% EtOAc/Hexane gradient) to afford tert-butyl 6-(((trifluoromethyl)sulfonyl)oxy)-2-azaspiro[3.3]hept-5-ene-2-carboxylate (4.7 g, 57.8% yield). LRMS (ES) 288.1 [M+H-Bu]⁺.

### Step 2: Preparation of tert-butyl 6-(4,4,5,5-tetramethyl-1,3,2-dioxaborolan-2-yl)-2-azaspiro[3.3]hept-5-ene-2-carboxylate

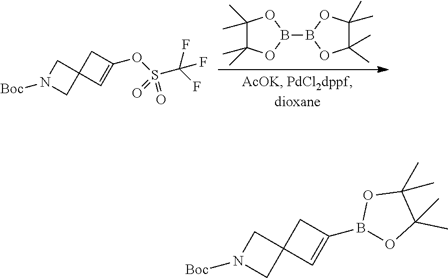

To a solution of tert-butyl 6-(((trifluoromethyl)sulfonyl)oxy)-2-azaspiro[3.3]hept-5-ene-2-carboxylate (2.7 g, 1 equiv) and potassium acetate (2 equiv) in dioxane (22 mL) was added 4,4,4′,4′,5,5,5′,5′-octamethyl-2,2′-bi(1,3,2-dioxaborolane) (1.3 equiv) and PdCl₂dppf (0.1 equiv), the mixture was stirred at 80° C. for 2 hour until reaction completed, the mixture was cooled, filtered through Celite, concentrated, and purified by silica gel chromatography (0-10% EtOAc/Hexanes gradient) to afford tert-butyl 6-(4,4,5,5-tetramethyl-1,3,2-dioxaborolan-2-yl)-2-azaspiro[3.3]hept-5-ene-2-carboxylate (1.8 g, 69.6% yield). LRMS (ES) 266.1 [M+H-Bu]⁺.

### Step 3: Preparation of tert-butyl 6-(2-fluoro-4-nitrophenyl)-2-azaspiro[3.3]hept-5-ene-2-carboxylate

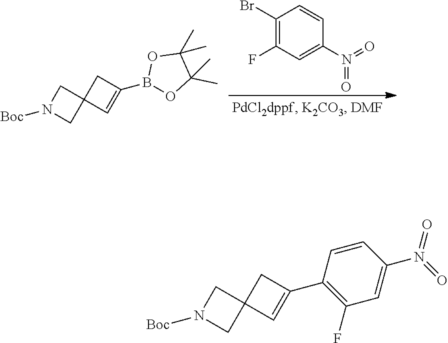 [SMILES: CC(C)(C)OC(=O)N1CC2(C=C(B3OC(C)(C)C(C)(C)O3)C2)C1 ; O=[N+]([O-])c1ccc(Br)c(F)c1 ; CC(C)(C)OC(=O)N1CC2(C=C(c3ccc([N+](=O)[O-])cc3F)C2)C1]

To a solution of tert-butyl 6-(4,4,5,5-tetramethyl-1,3,2-dioxaborolan-2-yl)-2-azaspiro[3.3]hept-5-ene-2-carboxylate (1.8 g, 1 equivequiv) and 1-bromo-2-fluoro-4-nitrobenzene (1.5 equivequiv) in DMF (30 mL), was added potassium carbonate (2 equiv) and PdCl₂dppf (0.1 equiv). The mixture was stirred at 80° for 2 hour until reaction completed, the mixture was cooled, filtered through Celite, concentrated and purified by silica gel chromatography (0-10% EtOAc/Hexanes gradient) to afford tert-butyl 6-(2-fluoro-4-nitrophenyl)-2-azaspiro[3.3]hept-5-ene-2-carboxylate (1.4 g, 74.8% yield) as a yellow solid. LRMS (ES) 279.1 [[M+H-Bu]⁺. ¹H NMR (400 MHz, DMSO-d₆) δ 8.14 (dd, J=10.5, 2.2 Hz, 1H), 8.10 (dd, J=8.5, 2.3 Hz, 1H), 7.59 (t, J=8.1 Hz, 1H), 6.79 (d, J=3.1 Hz, 1H), 4.12-4.03 (m, 4H), 3.07 (s, 2H), 1.39 (s, 9H).

### Step 4: Preparation of tert-butyl 6-(4-amino-2-fluorophenyl)-2-azaspiro[3.3]heptane-2-carboxylate

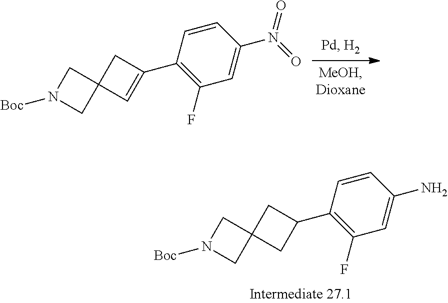 [SMILES: CC(C)(C)OC(=O)N1CC2(C=C(c3ccc([N+](=O)[O-])cc3F)C2)C1 ; CC(C)(C)OC(=O)N1CC2(CC(c3ccc(N)cc3F)C2)C1]

A solution of tert-butyl 6-(2-fluoro-4-nitrophenyl)-2-azaspiro[3.3]hept-5-ene-2-carboxylate (1.4 g, 1 equiv) in methanol (8 mL) and dioxane (8 mL) was purged under nitrogen for 5 minutes, followed by addition of Pd/C (0.31 equiv) slowly and purged for an additional 5 minutes. The mixture was filled with a balloon full of hydrogen, stirred at 24° C. for 16 h, filtered through Celite and concentrated to afford tert-butyl 6-(4-amino-2-fluorophenyl)-2-azaspiro[3.3]heptane-2-carboxylate (1.3 g, 99.6% yield). LRMS (ES) 251.1 [[M+H-Bu]⁺. ¹H NMR (400 MHz, DMSO-d₆) δ 6.90 (t, J=8.6 Hz, 1H), 6.32 (dd, J=8.3, 2.1 Hz, 1H), 6.24 (dd, J=13.0, 2.2 Hz, 1H), 5.18 (s, 2H), 3.95 (s, 2H), 3.73 (s, 2H), 3.36-3.26 (m, 1H), 2.43 (td, J=8.6, 2.9 Hz, 2H), 2.16 (td, J=9.5, 3.0 Hz, 2H), 1.37 (s, 9H).

Intermediate anilines in the following table were prepared in a similar manner as Intermediate 27.1, using the starting material and reagents as listed.

| Inter- | Starting Material | |
|---|---|---|
| mediate | And Reagents | Structure, Name and Data |
| 27.2 | tert-butyl 6-oxo-2- azaspiro[3.3] heptane- 2-carboxylate 1-bromo-4- nitrobenzene | 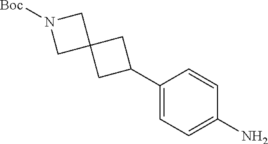 [SMILES: CC(C)(C)OC(=O)N1CC2(CC(c3ccc(N)cc3)C2)C1] |
| | | Intermediate 27.2: Tert-butyl 6-(4- |
| | | aminophenyl)-2-azaspiro[3.3]heptane-2- |
| | | carboxylate |
| | | LCMS-ES (POS.) m/z: 231.1 [M + H − Bu]⁺. |
| 27.3 | tert-butyl 2-oxo-6- azaspiro[3.4] octane-6- carboxylate 1-bromo-2- fluoro-4- nitrobenzene | 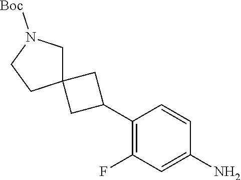 [SMILES: CC(C)(C)OC(=O)N1CCC2(CC(c3ccc(N)cc3F)C2)C1] |
| | | Intermediate 27.3: tert-butyl 2-(4-amino-2- |
| | | fluorophenyl)-6-azaspiro[3.4]octane-6- |
| | | carboxylate |
| | | LCMS-ES (POS.) m/z: 265.1 [M + H − Bu]⁺. |

### Example S

### Synthesis of Intermediates 28.1 to 27.8

### Step 1: Synthesis of diethyl (2-fluoro-4-nitrobenzyl)phosphonate

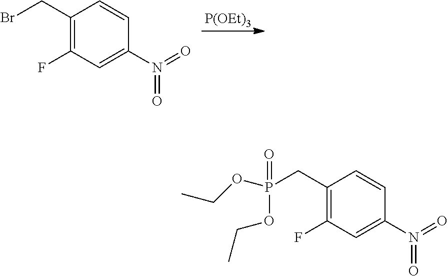 [SMILES: O=[N+]([O-])c1ccc(CBr)c(F)c1 ; CCOP(=O)(Cc1ccc([N+](=O)[O-])cc1F)OCC ; "P(OEt)3"]

1-(bromomethyl)-2-fluoro-4-nitrobenzene (3.01 g, 1 equiv) was mixed with triethyl phosphite (2 equiv) and the resulting solution was stirred at 120° C. for 40 minutes, cooled and purified by silica gel chromatography (10-100%% EtOAc/Hexanes gradient) to afford diethyl (2-fluoro-4-nitrobenzyl)phosphonate (3.37 g, 89.8% yield) as a yellow oil. LRMS (ES) 292.1 [M+H]. ¹H NMR (400 MHz, DMSO-d₆) δ 8.15-8.06 (m, 2H), 7.69-7.63 (m, 1H), 4.03-3.97 (m, 4H), 3.42 (dd, J=22.2, 1.2 Hz, 2H), 1.23-1.16 (m, 6H).

### Step 2: Synthesis of tert-butyl 6-(2-fluoro-4-nitrobenzylidene)-2-azaspiro[3.3]heptane-2-carboxylate

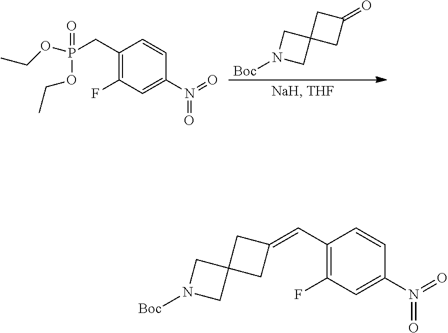 [SMILES: CCOP(=O)(Cc1ccc([N+](=O)[O-])cc1F)OCC ; CC(C)(C)OC(=O)N1CC2(CC(=O)C2)C1 ; CC(C)(C)OC(=O)N1CC2(CC(=Cc3ccc([N+](=O)[O-])cc3F)C2)C1]

To a solution of diethyl (2-fluoro-4-nitrobenzyl)phosphonate (2.2 g, 1 equiv) in THF (20 mL), was added tert-butyl 6-oxo-2-azaspiro[3.3]heptane-2-carboxylate (2 equiv) at 24° C., and sodium hydride (4 equiv) slowly at 0° C., the mixture was stirred at 0° C. for 1 h, slowly quenched with ice and water, and extracted with DCM. The combined organic layers were dried over sodium sulfate, concentrated and purified by silica gel chromatography (0-20% EtOAc/Hexanes gradient) to afford tert-butyl 6-(2-fluoro-4-nitrobenzylidene)-2-azaspiro[3.3]heptane-2-carboxylate (0.31 g, 11.6% yield) as a yellow oil. LRMS (ES) 393.2 [M+H-Bu]⁺.

### Step 3: Preparation of tert-butyl 6-(4-amino-2-fluorobenzyl)-2-azaspiro[3.3]heptane-2-carboxylate

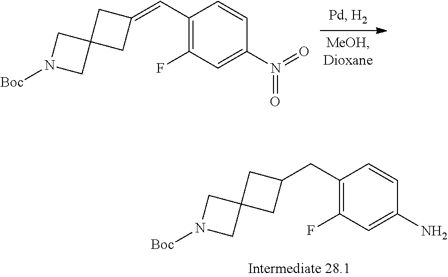 [SMILES: CC(C)(C)OC(=O)N1CC2(CC(=Cc3ccc([N+](=O)[O-])cc3F)C2)C1 ; CC(C)(C)OC(=O)N1CC2(CC(Cc3ccc(N)cc3F)C2)C1]

A solution of tert-butyl 6-(2-fluoro-4-nitrobenzylidene)-2-azaspiro[3.3]heptane-2-carboxylate (0.85 g, 1 equiv) in methanol (6 mL) and dioxane (6 mL) was purged under nitrogen for 5 minutes, added Pd (0.42 equiv) slowly, purged for an additional 5 minutes. The mixture was filled with a balloon full of hydrogen and stirred at 24° C. for 4 h, filtered through Celite and concentrated to afford tert-butyl 6-(4-amino-2-fluorobenzyl)-2-azaspiro[3.3]heptane-2-carboxylate (0.72 g, 92.8% yield). LRMS (ES) 265.1 [M+H-Bu]⁺.

Intermediate anilines in the following table were prepared in a similar manner as described above, using the starting material and reagents as listed.

| Intermediate | Starting Material | Structure, Name and Data |
|---|---|---|
| 28.2 | tert-butyl 2-oxo-6- azaspiro[3.4]octane-6- carboxylate 1-(bromomethyl)-2- fluoro-4-nitrobenzene | 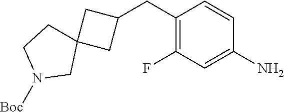 [SMILES: CC(C)(C)OC(=O)N1CCC2(CC(Cc3ccc(N)cc3F)C2)C1] |
| | | Intermediate 28.2: tert-butyl 2-(4-amino-2- |
| | | fluorobenzyl)-6-azaspiro[3.4]octane-6- |
| | | carboxylate |
| | | LCMS-ES (POS.) m/z: 279.1 [M + H − Bu]⁺. |
| | | ¹H NMR (400 MHz, DMSO-d₆) δ 6.83 (t, J = |
| | | 8.6 Hz, 1H), 6.31-6.22 (m, 2H), 5.13 (s, 2H), |
| | | 3.24-3.11 (m, 3H), 3.08 (d, J = 4.1 Hz, 1H), |
| | | 2.48 (s, 1H), 2.45-2.30 (m, 1H), 2.01-1.85 |
| | | (m, 2H), 1.84-1.57 (m, 4H), 1.37 (s, 9H), 1.22 |
| | | (s, 1H). |
| 28.3 | tert-butyl 8-oxo-3- azabicyclo[3.2.1]octane- 3-carboxylate 1-(bromomethyl)-4- nitrobenzene | 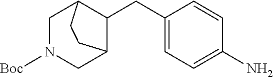 [SMILES: CC(C)(C)OC(=O)N1CC2CCC(C1)C2Cc1ccc(N)cc1] |
| | | Intermediate 28.3: tert-butyl 8-(4- |
| | | aminobenzyl)-3-azabicyclo[3.2.1]octane-3- |
| | | carboxylate |
| 28.4 | tert-butyl 6-oxo-3- azabicyclo[3.1.1]heptane- 3-carboxylate 1-(bromomethyl)-4- nitrobenzene | 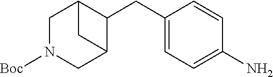 [SMILES: CC(C)(C)OC(=O)N1CC2CC(C1)C2Cc1ccc(N)cc1] |
| | | Intermediate 28.4: tert-butyl 6-(4- |
| | | aminobenzyl)-3-azabicyclo[3.1.1]heptane-3- |
| | | carboxylate |
| 28.5 | tert-butyl 7-oxo-4- azaspiro[2.5]octane-4- carboxylate 1-(bromomethyl)-4- nitrobenzene | 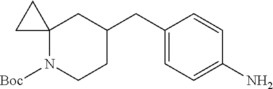 [SMILES: CC(C)(C)OC(=O)N1CCC(Cc2ccc(N)cc2)CC12CC2] |
| | | Intermediate 28.5: tert-butyl 7-(4- |
| | | aminobenzyl)-4-azaspiro[2.5]octane-4- |
| | | carboxylate |
| 28.6 | tert-butyl 5-oxo-2- azabicyclo[4.1.0]heptane- 2-carboxylate 1-(bromomethyl)-4- nitrobenzene | 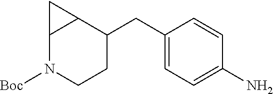 [SMILES: CC(C)(C)OC(=O)N1CCC(Cc2ccc(N)cc2)C2CC21] |
| | | Intermediate 28.6: tert-butyl 5-(4- |
| | | aminobenzyl)-2-azabicyclo[4.1.0]heptane-2- |
| | | carboxylate |
| 28.7 | tert-butyl 4-(2-fluoro-4- nitrobenzyl)piperidine-1- carboxylate 1-(bromomethyl)-2- fluoro-4-nitrobenzene | 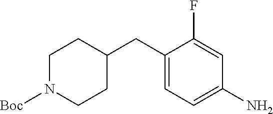 [SMILES: CC(C)(C)OC(=O)N1CCC(Cc2ccc(N)cc2F)CC1] |
| | | Intermediate 28.7: tert-butyl 4-(4-amino-2- |
| | | fluorobenzyl)piperidine-1-carboxylate |
| 28.8 | tert-butyl 3-oxo-8- azabicyclo[3.2.1]octane- 8-carboxylate 1-(bromomethyl)-4- nitrobenzene | 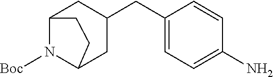 [SMILES: CC(C)(C)OC(=O)N1C2CCC1CC(Cc1ccc(N)cc1)C2] |
| | | Intermediate 28.8: tert-butyl 3-(4- |
| | | aminobenzyl)-8-azabicyclo[3.2.1]octane-8- |
| | | carboxylate |
| | | LCMS-ES (POS.) m/z: 261.1 [M + H − Boc]⁺. |

### Carbamate Synthesis

### Method 1: Synthesis of Carbamate from Aniline and Chloroformate

### Example F

### Preparation of 4-chlorobenzyl (4-((4-methyl-2-oxopiperazin-1-yl)methyl)phenyl)carbamate (Compound 28)

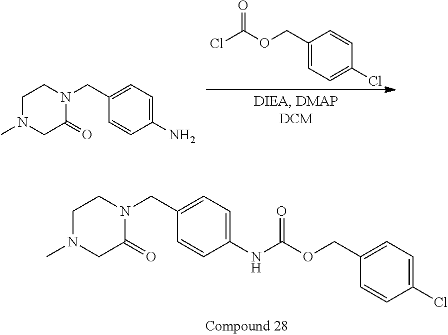 [SMILES: O=C(Cl)OCc1ccc(Cl)cc1 ; CN1CCN(Cc2ccc(N)cc2)C(=O)C1 ; CN1CCN(Cc2ccc(NC(=O)OCc3ccc(Cl)cc3)cc2)C(=O)C1]

To a mixture of aniline (88 mg, 0.40 mmol) in DCM (2 mL) was added DIEA (104 mg, 0.80 mmol), N,N-dimethylaniline (DMAP, 23 mg, 0.20 mmol) followed by 4-C₁-benzyl chloroformate (98 mg, 0.48 mmol). The reaction mix was stirred overnight and concentrated to dryness. The residue was purified on Agilent RP-HPLC on a Phenomenex, Gemini 5u C18 150×21.2 mm column eluting with a gradient of 10% ACN/Water to 100% ACN/Water over 40 min to give the desire product (40 mg, yield 65%) as an off-white solid. LRMS (ES) m/z: 388.1 [M+H]⁺. ¹H NMR (400 MHz, Methanol-d4) δ 7.47-7.24 (m, 6H), 7.22 (d, J=8.3 Hz, 2H), 5.17 (s, 2H), 4.57 (s, 2H), 3.34-3.26 (m, 2H), 3.17 (s, 2H), 2.68 (t, J=5.6 Hz, 2H), 2.35 (s, 3H).

Compounds in the following table were prepared in a similar manner as Compound 28, using the aniline as listed. Aniline intermediates were either purchased from commercial source or prepared according to above or to the procedures of published patent application WO 2021/159015.

| Co# | Aniline | Structure, Name and Data |
|---|---|---|
| 172 | 4-(pyridin-3- ylmethyl)aniline | 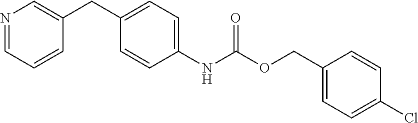 [SMILES: O=C(Nc1ccc(Cc2cccnc2)cc1)OCc1ccc(Cl)cc1] |
| | | Compound 172: 4-chlorobenzyl (4-(pyridin-3- |
| | | ylmethyl)phenyl)carbamate. LRMS (ES) m/z: 353.1 [[M + H]⁺. |
| | | ¹H NMR (400 MHz, Methanol-d₄) δ 8.44 (s, 1H), 7.67 (d, J = 7.9 |
| | | Hz, 1H), 7.38 (t, J = 7.7 Hz, 8H), 7.15 (d, J = 8.1 Hz, 2H), 5.16 (s, |
| | | 2H), 3.98 (s, 2H). |
| 216 | 4-(pyridin-4- ylmethyl)aniline | 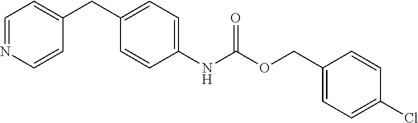 [SMILES: O=C(Nc1ccc(Cc2ccncc2)cc1)OCc1ccc(Cl)cc1] |
| | | Compound 216: 4-chlorobenzyl (4-(pyridin-4- |
| | | ylmethyl)phenyl)carbamate. |
| | | LRMS (ES) m/z: 353.1 [M + H]⁺. ¹H NMR (400 MHz, DMSO-d₆) |
| | | δ 9.75 (s, 1 H), 8.44 (d, J = 4.9 Hz, 2 H), 7.45 (s, 4 H), 7.39 (d, J = |
| | | 8.2 Hz, 2 H), 7.21 (d, J = 5.1 Hz, 2 H), 7.16 (d, J = 8.2 Hz, 2 H), |
| | | 5.13 (s, 2 H), 3.89 (s, 2 H). |
| 233 | 4-(pyridin-2- ylmethyl)aniline | 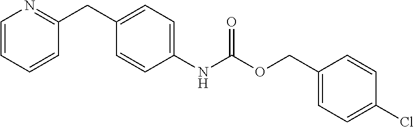 [SMILES: O=C(Nc1ccc(Cc2ccccn2)cc1)OCc1ccc(Cl)cc1] |
| | | Compound 233: 4-chlorobenzyl (4-(pyridin-2- |
| | | ylmethyl)phenyl)carbamate. LRMS (ES) m/z: 353.1 [M + H]⁺. |
| | | ¹H NMR (400 MHz, Methanol-d₄) δ 8.49-8.42 (m, 1H), 7.76 (td, |
| | | J = 7.7, 1.8 Hz, 1H), 7.40 (dd, J = 15.3, 9.2 Hz, 6H), 7.27 (dd, J = |
| | | 10.6, 6.8 Hz, 2H), 7.18 (d, J = 8.2 Hz, 2H), 5.16 (s, 2H), 4.10 (s, |
| | | 2H). |
| 307 | 4-benzylaniline | 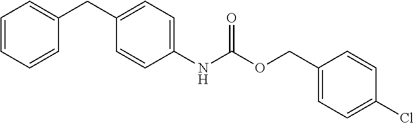 [SMILES: O=C(Nc1ccc(Cc2ccccc2)cc1)OCc1ccc(Cl)cc1] |
| | | Compound 307: 4-chlorobenzyl (4-(pyridin-3- |
| | | ylmethyl)phenyl)carbamate. LRMS (ES) m/z: 352.1 [M + H]. ¹H |
| | | NMR (400 MHz, Methanol-d₄) δ 7.40 (d, J = 5.9 Hz, 1H), 7.41- |
| | | 7.19 (m, 6H), 7.22-7.08 (m, 6H), 5.16 (s, 2H), 3.92 (s, 2H). |
| 236 | 1.1 | 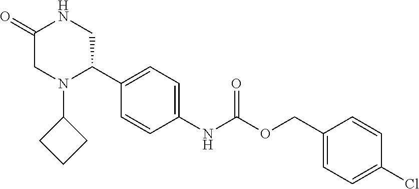 [SMILES: O=C1CN(C2CCC2)[C@@H](c2ccc(NC(=O)OCc3ccc(Cl)cc3)cc2)CN1] |
| | | Compound 236: 4-chlorobenzyl (S)-(4-(1-cyclobutyl-5- |
| | | oxopiperazin-2-yl)phenyl)carbamate. LRMS (ES) m/z: 414.1 |
| | | [M + H]⁺. ¹H NMR (400 MHz, Methanol-d₄) δ 7.48-7.25 (m, 8H), |
| | | 5.16 (s, 2H), 3.66-3.50 (m, 2H), 3.44-3.34 (m, 1H), 3.06-2.88 |
| | | (m, 2H), 2.00-1.88 (m, 2H), 1.75-1.40 (m, 4H), 1.29 (qd, J = |
| | | 7.0, 3.2 Hz, 1H). |
| 346 | 1.2 | 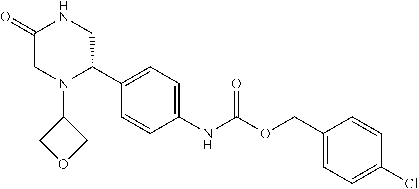 [SMILES: O=C1CN(C2COC2)[C@@H](c2ccc(NC(=O)OCc3ccc(Cl)cc3)cc2)CN1] |
| | | Compound 346: 4-chlorobenzyl (S)-(4-(1-(oxetan-3-yl)-5- |
| | | oxopiperazin-2-yl)phenyl)carbamate. LRMS (ES) m/z: 416.1 |
| | | [M + H]⁺. ¹H NMR (400 MHz, Chloroform-d) δ 7.39 (d, J = 17.3 |
| | | Hz, 1H), 7.35 (s, 4H), 7.35-7.20 (m, 2H), 6.92 (s, 1H), 5.17 (s, |
| | | 2H), 4.64 (t, J = 6.7 Hz, 1H), 4.46 (t, J = 6.6 Hz, 1H), 4.16 (t, J = |
| | | 6.5 Hz, 1H), 3.68 (t, J = 6.6 Hz, 1H), 3.60 (ddd, J = 20.6, 12.7, 7.7 |
| | | Hz, 2H), 3.46 (dd, J = 9.0, 3.7 Hz, 1H), 3.40-3.27 (m, 2H), 2.96 |
| | | (d, J = 16.7 Hz, 1H). |
| 304 | 1.3 | 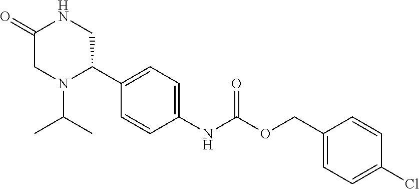 [SMILES: CC(C)N1CC(=O)NC[C@@H]1c1ccc(NC(=O)OCc2ccc(Cl)cc2)cc1] |
| | | Compound 304: 4-chlorobenzyl (S)-(4-(1-isopropyl-5- |
| | | oxopiperazin-2-yl)phenyl)carbamate. LRMS (ES) m/z: 402.1 |
| | | [M + H]⁺. ¹H NMR (400 MHz, Chloroform-d) δ 8.11 (s, 1H), 7.45- |
| | | 7.24 (m, 7H), 6.98 (s, 1H), 6.52 (s, 1H), 5.19 (s, 2H), 3.73 (dd, |
| | | J = 10.3, 3.7 Hz, 1H), 3.56 (d, J = 17.2 Hz, 1H), 3.44 (t, J = 11.1 |
| | | Hz, 1H), 3.26 (t, J = 14.5 Hz, 2H), 2.93 (p, J = 6.7 Hz, 1H), 1.05 |
| | | (d, J = 6.8 Hz, 3H), 0.79 (d, J = 6.4 Hz, 3H). |
| 249 | 5.1 | 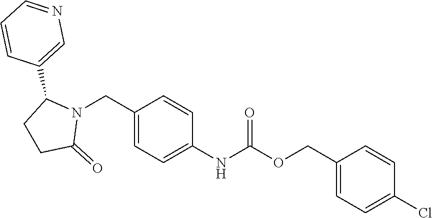 [SMILES: O=C(Nc1ccc(CN2C(=O)CC[C@@H]2c2cccnc2)cc1)OCc1ccc(Cl)cc1] |
| | | Compound 249: N-(4-{[(5R)-2-oxo-5-(pyridin-3-yl)pyrrolidin- |
| | | 1-yl]methyl}phenyl)carbamate. LCMS-APCI (POS.) m/z: |
| | | 436.1 [M + H]⁺. ¹H NMR (400 MHz, Methanol-d₄) δ 8.50 (d, J = |
| | | 4.8 Hz, 1H), 8.33 (s, 1H), 7.69 (dd, J = 8.0, 2.0 Hz, 1H), 7.50- |
| | | 7.28 (m, 7H), 7.01-6.88 (m, 2H), 5.16 (s, 2H), 4.80 (d, J = 14.8 |
| | | Hz, 1H), 4.61 (t, J = 7.0 Hz, 1H), 3.71 (d, J = 14.8 Hz, 1H), 2.74- |
| | | 2.44 (m, 3H), 2.01-1.87 (m, 1H). |
| 63 | 5.2 | 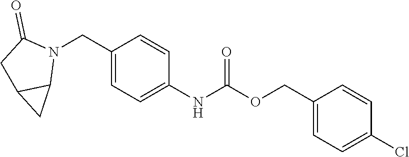 [SMILES: O=C(Nc1ccc(CN2C(=O)CC3CC32)cc1)OCc1ccc(Cl)cc1] |
| | | Compound 63: (4-chlorophenyl)methyl N-[4-({3-oxo-2- |
| | | azabicyclo[3.1.0]hexan-2-yl}methyl)phenyl]carbamate. |
| | | LCMS-ESI (POS.) m/z: 371.1 [M + H]⁺. ¹H NMR (400 MHz, |
| | | Methanol-d₄) δ 7.37-7.17 (m, 6H), 7.09 (d, J = 8.3 Hz, 2H), |
| | | 5.01 (s, 2H), 4.36-4.17 (m, 2H), 2.87 (ddt, J = 7.0, 5.1, 1.8 Hz, |
| | | 1H), 2.66 (dd, J = 18.1, 7.4 Hz, 1H), 2.22 (d, J = 18.1 Hz, 1H), |
| | | 1.41-1.27 (m, 1H), 0.65 (dt, J = 8.4, 5.5 Hz, 1H), −0.00 (td, J = |
| | | 5.4, 2.1 Hz, 1H). |
| 84 | 5.3 | 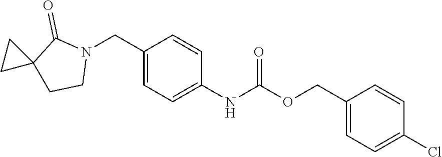 [SMILES: O=C(Nc1ccc(CN2CCC3(CC3)C2=O)cc1)OCc1ccc(Cl)cc1] |
| | | Compound 84: (4-chlorophenyl)methyl N-[4-({4-oxo-5- |
| | | azaspiro[2.4]heptan-5-yl}methyl)phenyl]carbamate. LCMS- |
| | | ESI (POS.) m/z: 385.1 [M + H]⁺. ¹H NMR (400 MHz, Methanol- |
| | | d₄) δ 7.33 (d, J = 8.0 Hz, 2H), 7.30-7.21 (m, 4H), 7.08 (d, J = |
| | | 8.1 Hz, 2H), 5.06 (s, 2H), 4.33 (s, 2H), 3.28 (m, 2H), 2.00 (t, J = |
| | | 7.4 Hz, 2H), 1.01 (q, J = 4.3, 3.8 Hz, 2H), 0.70 (t, J = 3.5 Hz, |
| | | 2H). |
| 123 | 5.4 | 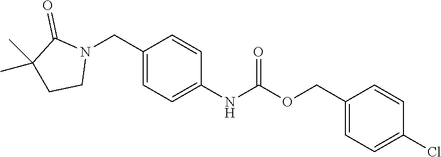 [SMILES: CC1(C)CCN(Cc2ccc(NC(=O)OCc3ccc(Cl)cc3)cc2)C1=O] |
| | | Compound 123: (4-chlorophenyl)methyl N-{4-[(3,3- |
| | | dimethyl-2-oxopyrrolidin-1-yl)methyl]phenyl}carbamate. |
| | | LCMS-ESI (POS.) m/z: 387.1 [M + H]⁺. ¹H NMR (400 MHz, |
| | | Methanol-d₄) δ 7.37-7.19 (m, 6H), 7.10-7.01 (m, 2H), 5.06 (s, |
| | | 2H), 4.28 (s, 2H), 3.09 (t, J = 6.9 Hz, 2H), 1.76 (t, J = 6.9 Hz, |
| | | 2H), 1.06 (d, J = 1.6 Hz, 6H). |
| 180 | 5.5 | 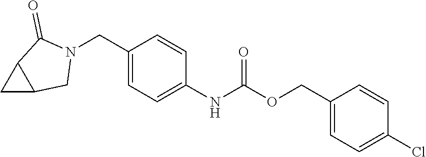 [SMILES: O=C(Nc1ccc(CN2CC3CC3C2=O)cc1)OCc1ccc(Cl)cc1] |
| | | Compound 180: (4-chlorophenyl)methyl N-[4-({2-oxo-3- |
| | | azabicyclo[3.1.0]hexan-3-yl}methyl)phenyl]carbamate. LCMS-ES |
| | | (Pos) m/z:371 [M + H]⁺. ¹H NMR (400 MHz, DMSO-d₆) δ 9.79 |
| | | (s, 1H), 7.49-7.38 (m, 6H), 7.06 (d, J = 8.1 Hz, 2H), 5.13 (s, |
| | | 2H), 4.15 (s, 2H), 3.32-3.28 (m, 1H), 3.08 (d, J = 10.5 Hz, 1H), |
| | | 1.89-1.77 (m, 2H), 1.07-1.01 (m, 1H), 0.46-0.40 (m, 1H). |

### Method 2: Preparation Isocyanate from Aniline and Triphosgene

### Example G

### Preparation of 4-chlorobenzyl (S)-(4-(1-methyl-5-oxopiperazin-2-yl)phenyl)carbamate (Compound 54)

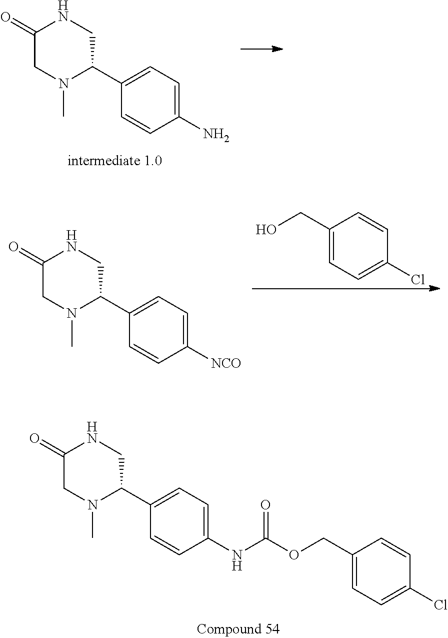

To a solution of triphosgene (60 mg, 0.20 mmol) in 2 mL of methylene chloride at 0° C. chilled with ice bath was added dropwise a solution of (S)-5-(4-aminophenyl)-4-methylpiperazin-2-one 2HCl salt (intermediate 1.0, 139 mg, 0.5 mmol) and diisopropylethylamine (0.35 mL, 2.0 mmol) in methylene chloride (4 mL) under nitrogen atmosphere while the internal temperature was kept under 5° C. After 0.5 h of stirring under ice bath, the resultant mixture was slowly added to a solution of 4-chlorobenzyl alcohol (109 mg, 0.75 mmol) and DMAP (6 mg, 0.05 mmol) in methylene chloride (2 mL). The mixture was warmed to room temperature and stirred at rt overnight and was concentrated to dryness. The residue was purified on Agilent RP-HPLC on a Phenomenex, Gemini 5u C18 150×21.2 mm column with a gradient of 10% ACN/Water to 100% ACN/Water over 40 min to give the desired product (115 mg, yield 62%) as an off-white solid. LRMS (ES) m/z: 374 [M+H]⁺. ¹H NMR (400 MHz, Methanol-d₄) δ 7.52-7.29 (m, 9H), 5.18 (s, 2H), 3.54 (d, J=17.1 Hz, 1H), 3.42 (s, 2H), 3.50-3.37 (m, 1H), 3.29 (d, J=8.2 Hz, 1H), 3.05 (d, J=17.0 Hz, 1H), 2.06 (s, 3H).

Compounds in the following table were prepared in a similar manner as Compound 54, using the aniline intermediates and alcohols as listed. Aniline intermediates were either purchased from commercial source or prepared according to the above procedures or to the procedures of published patent application WO 2021/159015.

| Co # | Aniline | Alcohol | Structure, Name and Data |
|---|---|---|---|
| 263 | 1.4 | (4- methoxyphenyl) methanol | 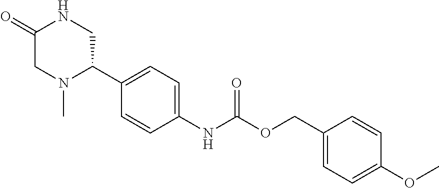 [SMILES: COc1ccc(COC(=O)Nc2ccc([C@H]3CNC(=O)CN3C)cc2)cc1] |
| | | | Compound 263: 4-methoxybenzyl (S)-(4-(1-methyl-5- |
| | | | oxopiperazin-2-yl)phenyl)carbamate. LRMS (ES) m/z: |
| | | | 370.1 [M + H]⁺. ¹H NMR (400 MHz, Methanol-d₄) δ 8.16 |
| | | | (s, 1H), 7.48 (d, J = 8.2 Hz, 2H), 7.40-7.24 (m, 4H), 6.97- |
| | | | 6.87 (m, 2H), 5.12 (s, 2H), 3.81 (s, 3H), 3.54 (d, J = 17.1 |
| | | | Hz, 1H), 3.43 (s, 1H), 3.50-3.37 (m, 1H), 3.29 (d, J = 7.8 |
| | | | Hz, 1H), 3.06 (d, J = 17.1 Hz, 1H), 2.06 (s, 3H). |
| 81 | 1.4 | (4-chloro-2- fluorophenyl) methanol | 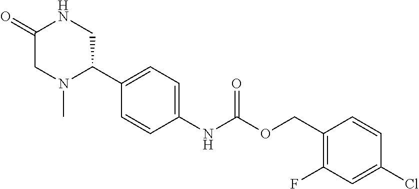 [SMILES: CN1CC(=O)NC[C@@H]1c1ccc(NC(=O)OCc2ccc(Cl)cc2F)cc1] |
| | | | Compound 81: 4-chloro-2-fluorobenzyl (S)-(4-(1- |
| | | | methyl-5-oxopiperazin-2-yl)phenyl)carbamate. LRMS |
| | | | (ES) m/z: 392.1 [M + H]⁺. ¹H NMR (400 MHz, Methanol- |
| | | | d₄) δ 7.51 (dd, J = 10.6, 8.0 Hz, 3H), 7.33 (d, J = 8.2 Hz, |
| | | | 2H), 7.29-7.21 (m, 2H), 5.23 (s, 2H), 3.57 (d, J = 17.0 |
| | | | Hz, 1H), 3.46 (d, J = 8.2 Hz, 2H), 3.37-3.28 (m, 2H), |
| | | | 3.10 (d, J = 17.1 Hz, 1H), 2.08 (s, 3H). |
| 278 | 1.4 | (4- fluorophenyl) methanol | 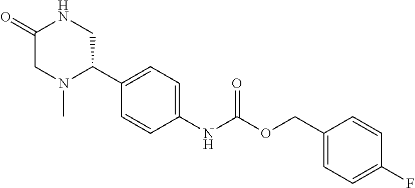 [SMILES: CN1CC(=O)NC[C@@H]1c1ccc(NC(=O)OCc2ccc(F)cc2)cc1] |
| | | | Compound 278: 4-fluorobenzyl (S)-(4-(1-methyl-5- |
| | | | oxopiperazin-2-yl)phenyl)carbamate. LRMS (ES) m/z: |
| | | | 358.1 [M + H]⁺. ¹H NMR (400 MHz, Methanol-d₄) δ 7.52- |
| | | | 7.43 (m, 2H), 7.32 (dd, J = 8.5, 1.8 Hz, 1H), 7.16- |
| | | | 7.07 (m, 1H), 5.18 (s, 2H), 3.58-3.41 (m, 1H), 3.41 (s, |
| | | | 1H), 3.29 (d, J = 9.5 Hz, 1H), 3.04 (d, J = 17.2 Hz, 1H), |
| | | | 2.05 (s, 3H). |
| 204 | 1.4 | (4- (difluoromethyl) phenyl) methanol | 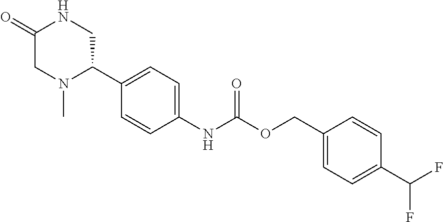 [SMILES: CN1CC(=O)NC[C@@H]1c1ccc(NC(=O)OCc2ccc(C(F)F)cc2)cc1] |
| | | | Compound 204: 4-(difluoromethyl)benzyl (S)-(4-(1- |
| | | | methyl-5-oxopiperazin-2-yl)phenyl)carbamate. LRMS |
| | | | (ES) m/z: 390.1 [M + H]⁺. ¹H NMR (400 MHz, Methanol- |
| | | | d₄) δ 7.57 (s, 4H), 7.50 (d, J = 8.1 Hz, 2H), 7.37-7.29 (m, |
| | | | 2H), 6.78 (t, J = 56 Hz, 1H), 5.26 (s, 2H), 4.62 (s, 1H), |
| | | | 3.54 (d, J = 17.0 Hz, 1H), 3.45 (d, J = 10.2 Hz, 1H), 3.42 |
| | | | (s, 2H), 3.29 (d, J = 8.8 Hz, 1H), 3.04 (d, J = 17.0 Hz, 1H), |
| | | | 2.06 (s, 3H). |
| 33 | N-(4- amino phenyl)- 1-phenyl methane- sulfonamide | (4- chlorophenyl) methanol | 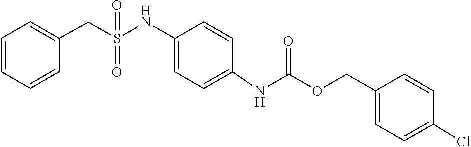 [SMILES: O=C(Nc1ccc(NS(=O)(=O)Cc2ccccc2)cc1)OCc1ccc(Cl)cc1] |
| | | | Compound 33: 4-chlorobenzyl (4- |
| | | | ((phenylmethyl)sulfonamido)phenyl)carbamate. |
| | | | ¹HNMR (300 MHz, DMSO-d₆) δ 9.75 (s, 1H), 7.51-7.31 |
| | | | (m, 9H), 7.26 (dd, J = 6.8, 2.9 Hz, 2H), 7.11 (d, J = 8.9 |
| | | | Hz, 2H), 5.14 (s, 2H), 4.38 (s, 2H). |
| 61 | N-(4- amino phenyl)-1- phenyl methane- sulfonamide | (4- chlorophenyl) methanol | 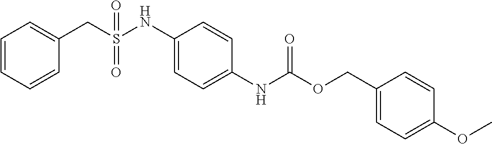 [SMILES: COc1ccc(COC(=O)Nc2ccc(NS(=O)(=O)Cc3ccccc3)cc2)cc1] |
| | | | Compound 61: 4-methoxybenzyl (4- |
| | | | ((phenylmethyl)sulfonamido)phenyl)carbamate. |
| | | | LRMS (ES) m/z: 427 [M + H]⁺. ¹HNMR (300 MHz, |
| | | | DMSO-d6) δ 9.63 (d, J = 12.2 Hz, 2H), 7.47-7.28 (m, |
| | | | 7H), 7.25 (dd, J = 6.8, 2.9 Hz, 2H), 7.09 (d, J = 8.9 Hz, |
| | | | 2H), 6.98-6.87 (m, 2H), 5.05 (s, 2H), 4.36 (s, 2H), 3.74 |
| | | | (s, 3H). |
| 242 | 3-(4- amino benzyl) oxazolidin- 2-one | (4- chlorophenyl) methanol | 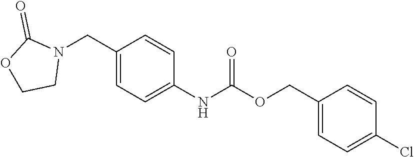 [SMILES: O=C(Nc1ccc(CN2CCOC2=O)cc1)OCc1ccc(Cl)cc1] |
| | | | Compound 242: (4-chlorophenyl)methyl N-{4-[(2-oxo- |
| | | | 1,3-oxazolidin-3-yl)methyl]phenyl}carbamate. LCMS- |
| | | | APCI (POS.) m/z: 343.1 [M + H]⁺. ¹H NMR (400 MHz, |
| | | | Methanol-d₄) δ 7.40-7.25 (m, 6H), 7.17-7.12 (m, 2H), |
| | | | 5.08 (s, 2H), 4.27 (s, 2H), 4.26-4.20 (m, 2H), 3.42-3.34 |
| | | | (m, 2H). |
| 281 | 1-(2- (4-amino phenyl) morpholino) ethan-1-one | (4- chlorophenyl) methanol | 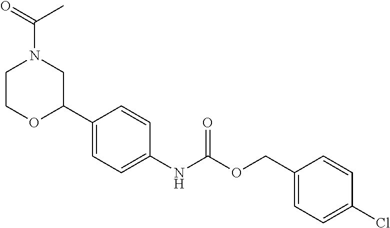 [SMILES: CC(=O)N1CCOC(c2ccc(NC(=O)OCc3ccc(Cl)cc3)cc2)C1] |
| | | | Compound 281: (4-chlorophenyl)methyl N-[4-(4- |
| | | | acetylmorpholin-2-yl)phenyl]carbamate. LCMS-APCI |
| | | | (POS.) m/z: 389.1 [M + H]⁺. ¹H NMR (400 MHz, |
| | | | Methanol-d₄) δ 7.54-7.21 (m, 8H), 5.16 (s, 2H), 4.52- |
| | | | 4.30 (m, 2H), 4.04 (dt, J = 11.3, 3.7 Hz, 1H), 3.90-3.76 |
| | | | (m, 1H), 3.66 (dtd, J = 28.2, 11.8, 2.8 Hz, 1H), 3.43-3.34 |
| | | | (m, 0.5H), 3.16 (dd, J = 13.4, 10.7 Hz, 0.5H), 2.88 (td, J = |
| | | | 12.8, 3.7 Hz, 0.5H), 2.69 (dd, J = 13.3, 10.7 Hz, 0.5H), |
| | | | 2.14, 2.13 (s, 3H). |
| 151 | 4-(4- amino phenyl) tetrahydro- 2H- thiopyran 1,1- dioxide | (4- chlorophenyl) methanol | 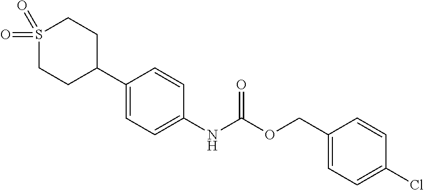 [SMILES: O=C(Nc1ccc(C2CCS(=O)(=O)CC2)cc1)OCc1ccc(Cl)cc1] |
| | | | Compound 151: (4-chlorobenzyl (4-(1,1- |
| | | | dioxidotetrahydro-2H-thiopyran-4- |
| | | | yl)phenyl)carbamate. LCMS-APCI (POS.) m/z: 394.1 |
| | | | [M + H]⁺. ¹H NMR (400 MHz, DMSO-d₆) δ 9.75 (s, 1H), |
| | | | 7.43 (d, J = 27.0 Hz, 6H), 7.17 (d, J = 8.2 Hz, 2H), 5.14 |
| | | | (s, 2H), 3.27 (s, 1H), 3.14-3.03 (m, 2H), 2.88 (q, J = |
| | | | 7.2 Hz, 2H), 2.06 (td, J = 9.4, 8.3, 3.3 Hz, 4H). |
| 262 | 4- (pyridin-4- ylmethyl) aniline | (4- chlorophenyl) methanol | 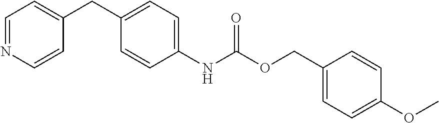 [SMILES: COc1ccc(COC(=O)Nc2ccc(Cc3ccncc3)cc2)cc1] |
| | | | Compound 262: 4-methoxybenzyl (4-(pyridin-4- |
| | | | ylmethyl)phenyl)carbamate. LCMS-APCI (POS.) m/z: |
| | | | 349.1 [M + H]⁺. ¹H NMR (400 MHz, DMSO-d₆) δ 9.65 (s, |
| | | | 1 H), 8.44 (d, J = 6.0 Hz, 2 H), 7.39 (d, J = 8.4 Hz, 2 H), |
| | | | 7.36 (d, J = 8.6 Hz, 2 H), 7.21 (d, J = 6.0 Hz, 2 H), 7.15 |
| | | | (d, J = 8.4 Hz, 2 H), 6.94 (d, J = 8.6 Hz, 2 H), 5.05 (s, 2H), |
| | | | 3.89 (s, 2 H), 3.75 (s, 3 H). |
| 306 | 4- (pyridin- 4-ylmethyl) aniline | (4- cyanophenyl) methanol | 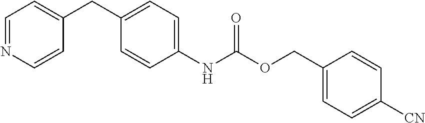 [SMILES: N#Cc1ccc(COC(=O)Nc2ccc(Cc3ccncc3)cc2)cc1] |
| | | | Compound 306: 4-cyanobenzyl (4-(pyridin-4- |
| | | | ylmethyl)phenyl)carbamate. LCMS-APCI (POS.) m/z: |
| | | | 344.1 ([M + H]⁺. ¹H NMR (400 MHz, DMSO-d₆) δ 9.83 |
| | | | (s, 1 H), 8.44 (d, J = 6.0 Hz, 2 H), 7.88 (d, J = 8.2 Hz, 2 H), |
| | | | 7.61 (d, J = 8.0 Hz, 2 H), 7.40 (d, J = 8.1 Hz, 2 H), 7.21 |
| | | | (d, J = 5.9 Hz, 2 H), 7.17 (d, J = 8.5 Hz, 2 H), 5.23 (s, 2 |
| | | | H), 3.90 (s, 2 H). |
| 86 | 4-(4- amino benzyl) morpholin- 3-one | (4- chlorophenyl) methanol | 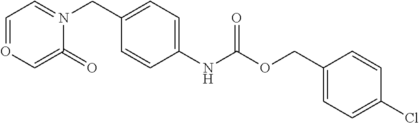 |
| | | | Compound 86: (4-chlorophenyl)methyl N-{4-[(3- |
| | | | oxomorpholin-4-yl)methyl]phenyl}carbamate. LCMS- |
| | | | ESI (POS.) m/z: 375.0 [M + H]⁺. 1H NMR (400 MHz, |
| | | | DMSO-d6) δ 9.81 (s, 1H), 7.45 (d, J = 13.7 Hz, 6H), 7.27- |
| | | | 7.07 (m, 2H), 5.14 (s, 2H), 4.47 (s, 2H), 4.10 (s, 2H), |
| | | | 3.79 (dd, J = 5.9, 4.3 Hz, 2H), 3.22 (dd, J = 6.0, 4.3 Hz, |
| | | | 2H). |
| 174 | 4-(4- amino benzyl) morpholin- 3-one | (4- methoxyphenyl) methanol | 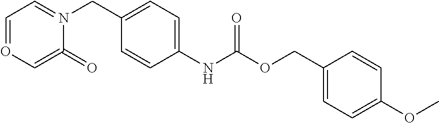 |
| | | | Compound 174: (4-methoxyphenyl)methyl N-{4-[(3- |
| | | | oxomorpholin-4-yl)methyl]phenyl}carbamate. LCMS- |
| | | | ESI (POS.) m/z: 371.0 [M + H]⁺. 1H NMR (300 MHz, |
| | | | DMSO-d6) δ 9.71 (s, 1H), 7.48-7.28 (m, 4H), 7.23-7.08 |
| | | | (m, 2H), 7.06-6.80 (m, 2H), 5.07 (s, 2H), 4.47 (s, 2H), |
| | | | 4.10 (s, 2H), 3.87-3.64 (m, 5H), 3.22 (dd, J = 6.0, 4.3 |
| | | | Hz, 2H). |
| 247 | 3.1 | (4- chlorophenyl) methanol | 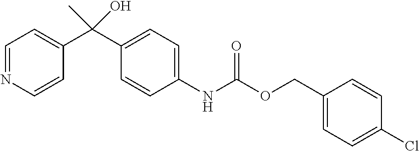 [SMILES: CC(O)(c1ccncc1)c1ccc(NC(=O)OCc2ccc(Cl)cc2)cc1] |
| | | | Compound 247: 4-chlorobenzyl (4-(1-hydroxy-1- |
| | | | (pyridin-4-yl)ethyl)phenyl)carbamate. LRMS (ES) |
| | | | m/z: 383 [M + H]⁺. ¹H NMR (400 MHz, Methanol-d₄) δ |
| | | | 8.46 (d, J = 5.3 Hz, 2H), 7.53 (d, J = 5.2 Hz, 2H), 7.45- |
| | | | 7.35 (m, 8H), 5.16 (s, 2H), 1.91 (s, 3H). |
| 296 | 3.1 | oxazol-5- ylmethanol | 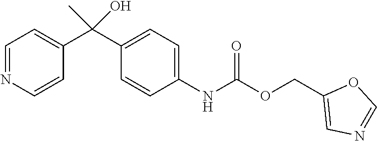 [SMILES: CC(O)(c1ccncc1)c1ccc(NC(=O)OCc2cnco2)cc1] |
| | | | Compound 296: oxazol-5-ylmethyl (4-(1-hydroxy-1- |
| | | | (pyridin-4-yl)ethyl)phenyl)carbamate. LRMS (ES) |
| | | | m/z: 340 [M + H]⁺. ¹H NMR (400 MHz, Methanol-d₄) δ |
| | | | 8.46 (d, J = 5.3 Hz, 2H), 8.23 (s, 1H), 7.53 (d, J = 5.0 |
| | | | Hz, 2H), 7.46-7.32 (m, 4H), 7.25 (s, 1H), 5.24 (s, 2H), |
| | | | 1.92 (s, 3H). |
| 329 | 3.1 | thiazol-5- ylmethanol | 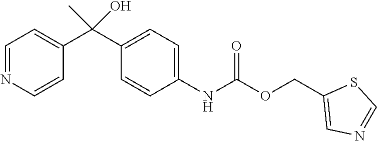 [SMILES: CC(O)(c1ccncc1)c1ccc(NC(=O)OCc2cncs2)cc1] |
| | | | Compound 329: thiazol-5-ylmethyl (4-(1-hydroxy-1- |
| | | | (pyridin-4-yl)ethyl)phenyl)carbamate. LRMS (ES) |
| | | | m/z: 356 [M + H]⁺. ¹H NMR (400 MHz, Methanol-d₄) δ |
| | | | 9.02 (s, 1H), 8.46 (d, J = 5.2 Hz, 2H), 7.96 (s, 1H), 7.53 |
| | | | (d, J = 5.2 Hz, 2H), 7.46-7.329 (m, 4H), 5.43 (s, 2H), |
| | | | 1.92 (s, 3H). |
| | tert-butyl 4-(4- amino benzyl) piperidine- 1-carboxylate | (4- chlorophenyl) methanol | 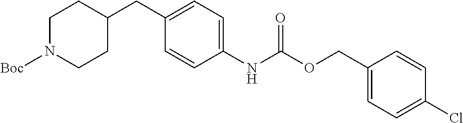 [SMILES: CC(C)(C)OC(=O)N1CCC(Cc2ccc(NC(=O)OCc3ccc(Cl)cc3)cc2)CC1] |
| | | | Intermediate 6.1: tert-butyl 4-(4-((((4- |
| | | | chlorobenzyl)oxy)carbonyl)amino)benzyl)piperidine- |
| | | | 1-carboxylate. LRMS (ES) m/z: 403[M + H-tBu]⁺. ¹H |
| | | | NMR (400 MHz, Methanol-d₄) δ 7.58-7.23 (m, 6H), 7.23- |
| | | | 6.88 (m, 2H), 5.16 (s, 2H), 4.04 (d, J = 13.4 Hz, 2H), |
| | | | 2.69 (t, J = 12.8 Hz, 2H), 2.59-2.34 (m, 2H), 1.66 (dd, J = |
| | | | 33.1, 11.7 Hz, 3H), 1.46 (d, J = 3.3 Hz, 9H), 1.09 (q, J = |
| | | | 12.4 Hz, 2H). |
| 67 | 4-((1- (oxetan-3- yl)piperidin- 4-yl)methyl) aniline | (4- methoxyphenyl) methanol | 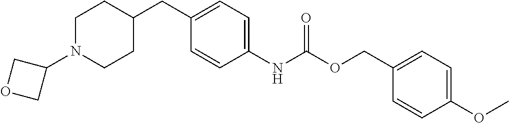 [SMILES: COc1ccc(COC(=O)Nc2ccc(CC3CCN(C4COC4)CC3)cc2)cc1] |
| | | | Compound 67: 4-methoxybenzyl (4-((1-(oxetan-3- |
| | | | yl)piperidin-4-yl)methyl)phenyl)carbamate. LCMS- |
| | | | ESI (POS.) m/z: 411.0 [M + H]⁺. ¹H NMR (300 MHz, |
| | | | DMSO-d6) δ 9.60 (d, J = 4.6 Hz, 1H), 7.35 (s, 4H), 7.05 |
| | | | (s, 2H), 6.94 (s, 2H), 5.06 (s, 2H), 4.49 (d, J = 6.6 Hz, 2H), |
| | | | 4.38 (s, 2H), 3.75 (t, J = 4.1 Hz, 3H), 2.61 (d, J = 8.2 Hz, |
| | | | 2H), 2.42 (s, 3H), 1.64 (s, 2H), 1.52 (d, J = 12.6 Hz, 3H), |
| | | | 1.16 (s, 2H). |
| | tert- butyl 4-(4- amino phenyl) piperidine-1- carboxylate | (4- chlorophenyl) methanol | 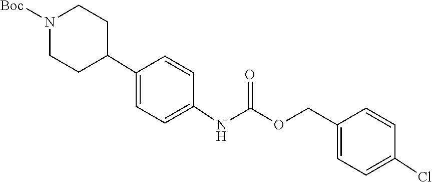 [SMILES: CC(C)(C)OC(=O)N1CCC(c2ccc(NC(=O)OCc3ccc(Cl)cc3)cc2)CC1] |
| | | | Intermediate 7.1: tert-butyl 4-[4-({[(4- |
| | | | chlorophenyl)methoxy]carbonyl}amino)phenyl]piperidine- |
| | | | 1-carboxylate. LCMS-APCI (POS.) m/z: 445.1 |
| | | | [M + H]⁺. |
| | tert-butyl 4-(4- amino benzyl) piperidine-1- carboxylate | oxazol-5- ylmethanol | 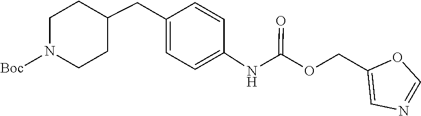 [SMILES: CC(C)(C)OC(=O)N1CCC(Cc2ccc(NC(=O)OCc3cnco3)cc2)CC1] |
| | | | Intermediate 8.1: tert-butyl 4-(4-(((oxazol-5- |
| | | | ylmethoxy)carbonyl)amino)benzyl)piperidine-1- |
| | | | carboxylate. LRMS (ES) m/z: 360 [M + H-tBu]⁺. ¹H NMR |
| | | | (400 MHz, Methanol-d₄) δ 8.24 (s, 1H), 7.35 (d, J = 8.0 |
| | | | Hz, 2H), 7.25 (s, 1H), 7.10 (d, J = 8.1 Hz, 2H), 5.25 (s, |
| | | | 2H), 4.05 (d, J = 13.2 Hz, 2H), 2.70 (s, 2H), 2.51 (d, J = |
| | | | 7.0 Hz, 2H), 1.63 (d, J = 13.8 Hz, 3H), 1.46 (d, J = 1.4 Hz, |
| | | | 9H), 1.10 (qd, J = 12.5, 4.3 Hz, 2H). |
| | tert- butyl 4-(4- amino phenyl) piperidine-1- carboxylate | oxazol-5- ylmethanol | 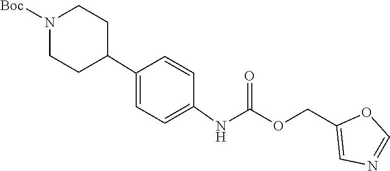 [SMILES: CC(C)(C)OC(=O)N1CCC(c2ccc(NC(=O)OCc3cnco3)cc2)CC1] |
| | | | Intermediate 9.1: tert-butyl 4-(4-(((oxazol-5- |
| | | | ylmethoxy)carbonyl)amino)phenyl)piperidine-1- |
| | | | carboxylate. LRMS (ES) m/z: 402.1 M + H]⁺. |
| | tert- butyl 4-(4- amino benzyl) piperidine-1- carboxylate | pyridin-4- ylmethanol | 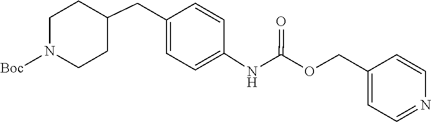 [SMILES: CC(C)(C)OC(=O)N1CCC(Cc2ccc(NC(=O)OCc3ccncc3)cc2)CC1] |
| | | | Intermediate 10.1: tert-butyl 4-(4-(((pyridin-4- |
| | | | ylmethoxy)carbonyl)amino)benzyl)piperidine-1- |
| | | | carboxylate. LCMS-APCI (POS.) m/z: 426.0 [M + H]⁺. |
| 121 | 2.1 | oxazol-5- ylmethanol | 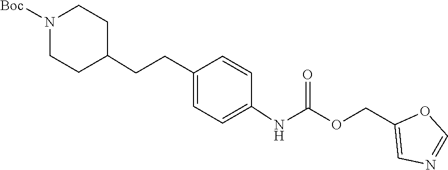 [SMILES: CC(C)(C)OC(=O)N1CCC(CCc2ccc(NC(=O)OCc3cnco3)cc2)CC1] |
| | | | Compound 121: tert-butyl 4-(4-(((oxazol-5- |
| | | | ylmethoxy)carbonyl)amino)phenethyl)piperidine-1- |
| | | | carboxylate. LRMS (ES) m/z: 430 [M + H]⁺. ¹H NMR |
| | | | (400 MHz, Methanol-d₄) δ 8.24 (s, 1H), 7.34 (d, J = 8.1 |
| | | | Hz, 2H), 7.25 (s, 1H), 7.12 (d, J = 8.5 Hz, 2H), 5.24 (s, |
| | | | 2H), 4.06 (dt, J = 13.2, 2.8 Hz, 2H), 2.72 (m, 2H), 2.62 |
| | | | (dd, J = 9.0, 6.7 Hz, 2H), 1.86-1.69 (m, 2H), 1.66-1.45 |
| | | | (m, 3H), 1.47 (s, 9H), 1.10 (qd, J = 12.6, 4.3 Hz, 2H). |
| | 4- bromo aniline | (4- chlorophenyl) methanol | 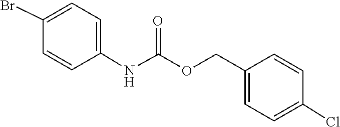 [SMILES: O=C(Nc1ccc(Br)cc1)OCc1ccc(Cl)cc1] |
| | | | Intermediate 12.1: 4-chlorobenzyl (4- |
| | | | bromophenyl)carbamate. LRMS (ES) m/z: 341[M + H]⁺. |
| | 4.1 | (4- chlorophenyl) methanol | 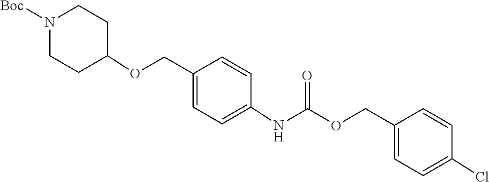 [SMILES: CC(C)(C)OC(=O)N1CCC(OCc2ccc(NC(=O)OCc3ccc(Cl)cc3)cc2)CC1] |
| | | | Intermediate 13.1: tert-butyl 4-{[4-({[(4- |
| | | | chlorophenyl)methoxy\|carbonyl}amino)phenyl]methoxy} |
| | | | piperidine-1-carboxylate. |
| | | | LCMS-APCI (POS.) m/z: 375.1 [M + H-Boc]⁺. |
| | | | 1H NMR (400 MHz, Methanol-d4) δ 7.45-7.34 (m, 6H), |
| | | | 7.26 (d, J = 8.2 Hz, 2H), 5.15 (s, 2H), 4.50 (s, 2H), 3.73 |
| | | | (dt, J = 11.0, 4.5 Hz, 2H), 3.60 (tt, J = 8.0, 3.5 Hz, 1H), |
| | | | 3.11 (m, 2H), 1.90-1.81 (m, 2H), 1.51 (m, 2H), 1.45 (s, |
| | | | 9H). |
| 288 | 4.2 | (4- chlorophenyl) methanol | 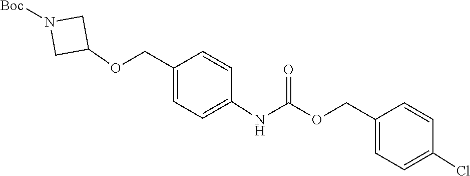 [SMILES: CC(C)(C)OC(=O)N1CC(OCc2ccc(NC(=O)OCc3ccc(Cl)cc3)cc2)C1] |
| | | | Compound 288: tert-butyl 3-{[4-({[(4- |
| | | | chlorophenyl)methoxy]carbonyl}amino)phenyl]methoxy} |
| | | | azetidine-1-carboxylate. LCMS-APCI (POS.) m/z: |
| | | | 347.1 [M + H-Boc]⁺. |
| | | | 1H NMR (400 MHz, Methanol-d4) δ 7.48-7.23 (m, 8H), |
| | | | 5.16 (s, 2H), 4.42 (s, 2H), 4.34 (s, 2H), 4.09-3.97 (m, |
| | | | 2H), 3.79-3.68 (m, 2H), 1.42 (s, 9H). |
| 291 | 4.3 | (4- methoxyphenyl) methanol | 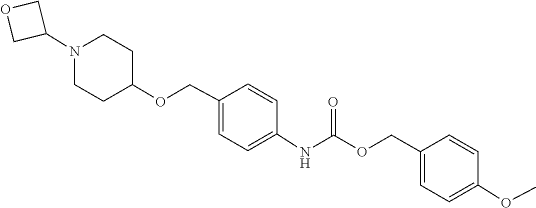 [SMILES: COc1ccc(COC(=O)Nc2ccc(COC3CCN(C4COC4)CC3)cc2)cc1] |
| | | | Compound 291: 4-methoxybenzyl (4-(((1-(oxetan-3- |
| | | | yl)piperidin-4-yl)oxy)methyl)phenyl)carbamate. |
| | | | LCMS-APCI (POS.) m/z: 427.2 [M + H]⁺. |
| | | | 1H NMR (400 MHz, Methanol-d4) δ 7.40 (d, J = 8.2 Hz, |
| | | | 2H), 7.36-7.32 (m, 2H), 7.25 (d, J = 8.4 Hz, 2H), 6.94- |
| | | | 6.89 (m, 2H), 5.10 (s, 2H), 4.67 (t, J = 6.7 Hz, 2H), 4.58 |
| | | | (t, J = 6.3 Hz, 2H), 4.48 (s, 2H), 3.79 (s, 3H), 3.51 (p, J = |
| | | | 6.4 Hz, 2H), 2.66-2.57 (m, 2H), 2.11 (t, J = 9.9 Hz, |
| | | | 2H), 1.98-1.88 (m, 2H), 1.73-1.60 (m, 2H). |
| 217 | 4.4 | (4- methoxyphenyl) methanol | 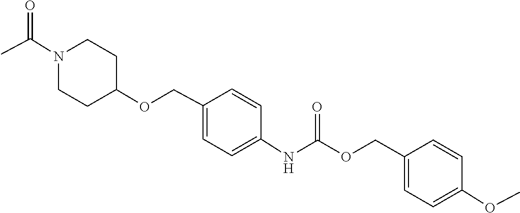 [SMILES: COc1ccc(COC(=O)Nc2ccc(COC3CCN(C(C)=O)CC3)cc2)cc1] |
| | | | Compound 217: 4-methoxybenzyl (4-(((1- |
| | | | acetylpiperidin-4-yl)oxy)methyl)phenyl)carbamate. |
| | | | LCMS-APCI (POS.) m/z: 413.1 [M + H]⁺. ¹H NMR (400 |
| | | | MHz, Methanol-d₄) δ 7.41 (d, J = 8.2 Hz, 2H), 7.34 (d, J = |
| | | | 8.3 Hz, 2H), 7.26 (d, J = 8.2 Hz, 2H), 6.91 (d, J = 8.0 |
| | | | Hz, 2H), 5.09 (s, 2H), 4.50 (s, 2H), 3.92-3.83 (m, 1H), |
| | | | 3.78 (s, 3H), 3.76-3.62 (m, 2H), 3.31-3.23 (m, 2H), |
| | | | 2.08 (s, 3H), 1.94-1.79 (m, 2H), 1.65-1.48 (m, 2H). |
| | tert- butyl (4- amino benzyl) carbamate | 4- chlorobenzyl alcohol | 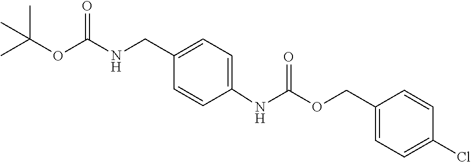 [SMILES: CC(C)(C)OC(=O)NCc1ccc(NC(=O)OCc2ccc(Cl)cc2)cc1] |
| | | | Intermediate 15.1: (4-chlorophenyl)methyl N-(4- |
| | | | {[(tert- |
| | | | butoxycarbonyl)amino]methyl}phenyl)carbamate. |
| | | | LCMS-ESI (NEG.) m/z: 389.0 [M − H]⁻. |
| | tert- butyl (S)-(1-(4- amino phenyl) ethyl) carbamate | 4- chlorobenzyl alcohol | 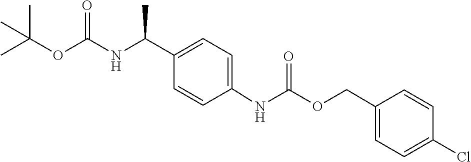 [SMILES: C[C@H](NC(=O)OC(C)(C)C)c1ccc(NC(=O)OCc2ccc(Cl)cc2)cc1] |
| | | | Intermediate 16.1: (4-chlorophenyl)methyl N-(4-[(1S)- |
| | | | 1-[(tert- |
| | | | butoxycarbonyl)amino]ethyl]phenyl}carbamate. |
| | | | LCMS-APCI (POS.) m/z: 405.1 [M + H]⁺. |
| | tert- butyl (4-amino benzyl) (methyl) carbamate | 4- chlorobenzyl alcohol | 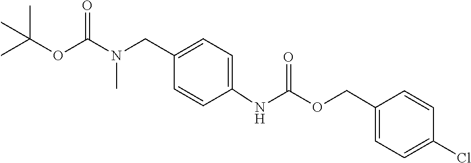 [SMILES: CN(Cc1ccc(NC(=O)OCc2ccc(Cl)cc2)cc1)C(=O)OC(C)(C)C] |
| | | | Intermediate 17.1: (4-chlorophenyl)methyl N-(4- |
| | | | {[(tert- |
| | | | butoxycarbonyl)(methyl)amino methyl}phenyl)carbamate. |
| | | | LCMS-ES (POS.) m/z: 427.0 [M + Na]⁺. |
| | tert- butyl (4- amino benzyl) (methyl) carbamate | 4- chlorobenzyl alcohol | 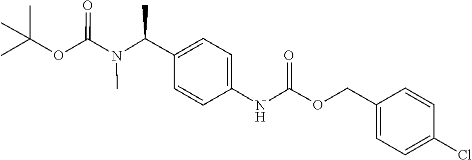 [SMILES: C[C@@H](c1ccc(NC(=O)OCc2ccc(Cl)cc2)cc1)N(C)C(=O)OC(C)(C)C] |
| | | | Intermediate 18.1: tert-butyl (S)-(1-(4-((((4- |
| | | | chlorobenzyl)oxy)carbonyl)amino)phenyl)ethyl)(methyl) |
| | | | carbamate. LCMS-ES (POS.) m/z: 419.0 [M + H]⁺. |
| | tert- butyl (4-amino benzyl) carbamate | 1,3-oxazol- 5- ylmethanol | 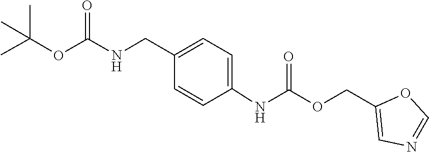 [SMILES: CC(C)(C)OC(=O)NCc1ccc(NC(=O)OCc2cnco2)cc1] |
| | | | Intermediate 19.1: 1,3-oxazol-5-ylmethyl N-(4-{[(tert- |
| | | | butoxycarbonyl)amino methyl}phenyl)carbamate. |
| | | | LCMS-ESI (POS.) m/z: 348.1 [M + H]⁺. |
| | tert- butyl (S)-(1- (4-amino phenyl) ethyl) carbamate | 1,3-oxazol- 5- ylmethanol | 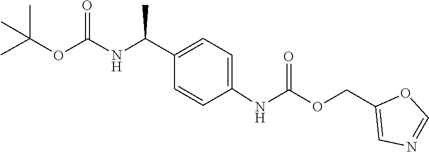 [SMILES: C[C@H](NC(=O)OC(C)(C)C)c1ccc(NC(=O)OCc2cnco2)cc1] |
| | | | Intermediate 20.1: 1,3-oxazol-5-ylmethyl N-{4-[(1S)-1- |
| | | | [(tert- |
| | | | butoxycarbonyl)amino]ethyl]phenyl}carbamate. |
| | | | LCMS-APCI (POS.) m/z: 362.2 [M + H]⁺. |
| 322 | 4-[(5- methyl- 1,3,4- oxadiazol-2- yl)methyl] aniline | 4- chlorobenzyl alcohol | 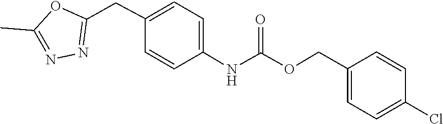 [SMILES: Cc1nnc(Cc2ccc(NC(=O)OCc3ccc(Cl)cc3)cc2)o1] |
| | | | Compound 322: (4-chlorophenyl)methyl N-{4-[(5- |
| | | | methyl-1,3,4-oxadiazol-2- |
| | | | yl)methyl]phenyl}carbamate. LCMS-APCI (POS.) m/z: |
| | | | 358.3 [M + H]⁺. ¹H NMR (400 MHz, DMSO-d₆) δ 9.80 (s, |
| | | | 1H), 7.49-7.44 (m, 4H), 7.42 (d, J = 7.9 Hz, 2H), 7.21 |
| | | | (d, J = 8.1 Hz, 2H), 5.13 (s, 2H), 4.13 (s, 2H), 2.43 (s, 3H). |
| | ethyl 2-(4- amino phenyl) acetate | (4- chlorophenyl) methanol | 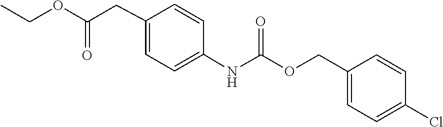 [SMILES: CCOC(=O)Cc1ccc(NC(=O)OCc2ccc(Cl)cc2)cc1] |
| | | | Intermediate 21.1: ethyl 2-(4-((((4- |
| | | | chlorobenzyl)oxy)carbonyl)amino)phenyl)acetate. |
| | | | LRMS (ES) m/z: 348.1 [M + H]⁺. |
| | 4- amino benzenesulfonyl chloride | (4- chlorophenyl) methanol | 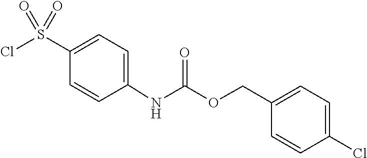 [SMILES: O=C(Nc1ccc(S(=O)(=O)Cl)cc1)OCc1ccc(Cl)cc1] |
| | | | Intermediate 22.1: (4-chlorophenyl)methyl N-[4- |
| | | | (chlorosulfonyl)phenyl]carbamate. 1H NMR (400 |
| | | | MHz, DMSO-d6) δ 9.87 (s, 1H), 7.50 (d, J = 8.6 Hz, 2H), |
| | | | 7.40-7.30 (m, 6H), 5.14 (s, 2H). |
| | 27.2 | 1,3-oxazol- 5-ylmethanol | 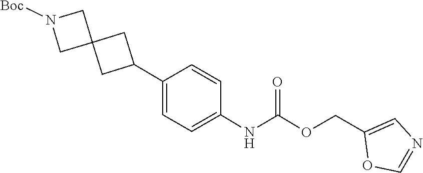 [SMILES: CC(C)(C)OC(=O)N1CC2(CC(c3ccc(NC(=O)OCc4cnco4)cc3)C2)C1] |
| | | | Intermediate 29.1: tert-butyl 6-(4-((2-(oxazol-5- |
| | | | ylmethoxy)-2-oxoethyl)amino)phenyl)-2- |
| | | | azaspiro[3.3]heptane-2-carboxylate |
| | | | LCMS-ESI (POS.) m/z: 358.1 [M + H-Bu]⁺ |
| | 27.3 | 1,3-oxazol- 5-ylmethanol | 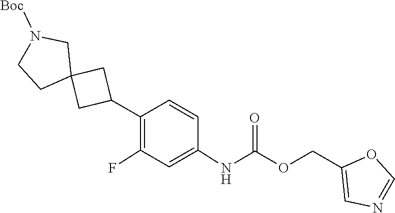 [SMILES: CC(C)(C)OC(=O)N1CCC2(CC(c3ccc(NC(=O)OCc4cnco4)cc3F)C2)C1] |
| | | | Intermediate 29.2: tert-butyl 2-(2-fluoro-4-(((oxazol- |
| | | | 5-ylmethoxy)carbonyl)amino)phenyl)-6- |
| | | | azaspiro[3.4]octane-6-carboxylate |
| | | | LCMS-ESI (POS.) m/z: 390.1 [M + H-Bu]⁺ |
| | tert- butyl (R)-3-(4- amino phenyl) piperidine-1- carboxylate | 1,3-oxazol- 5-ylmethanol | 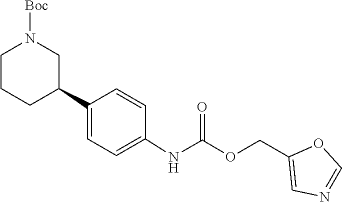 [SMILES: CC(C)(C)OC(=O)N1CCC[C@H](c2ccc(NC(=O)OCc3cnco3)cc2)C1] |
| | | | Intermediate 29.3: tert-butyl (R)-3-(4-(((oxazol-5- |
| | | | ylmethoxy)carbonyl)amino)phenyl)piperidine-1- |
| | | | carboxylate |
| | | | LCMS-ESI (POS.) m/z: 346.1 [M + H-Bu]⁺ |
| | 28.2 | 1,3-oxazol- 5-ylmethanol | 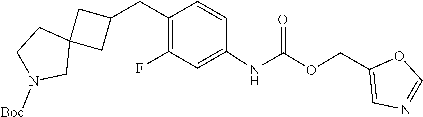 [SMILES: CC(C)(C)OC(=O)N1CCC2(CC(Cc3ccc(NC(=O)OCc4cnco4)cc3F)C2)C1] |
| | | | Intermediate 29.4: tert-butyl 2-(2-fluoro-4-(((oxazol- |
| | | | 5-ylmethoxy)carbonyl)amino)benzyl)-6- |
| | | | azaspiro[3.4]octane-6-carboxylate |
| | | | LCMS-ESI (POS.) m/z: 460.1 [M + H]⁺ |
| | | | ¹H NMR (400 MHz, DMSO-d₆) δ 9.94 (s, 1H), 8.42 (s, |
| | | | 1H), 7.36-7.23 (m, 2H), 7.22-7.05 (m, 2H), 5.22 (s, |
| | | | 2H), 3.23-3.12 (m, 3H), 3.09 (s, 1H), 2.68-2.57 (m, |
| | | | 2H), 2.05-1.83 (m, 2H), 1.84-1.75 (m, 1H), 1.74- |
| | | | 1.62 (m, 4H), 1.37 (s, 9H). |
| | tert- butyl (S)-3-(4- amino phenyl) piperidine-1- carboxylate | 1,3-oxazol- 5-ylmethanol | 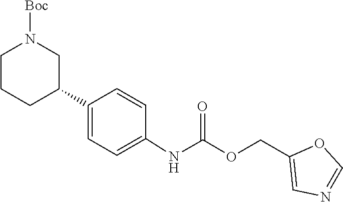 [SMILES: CC(C)(C)OC(=O)N1CCC[C@@H](c2ccc(NC(=O)OCc3cnco3)cc2)C1] |
| | | | Intermediate 29.5: tert-butyl (S)-3-(4-(((oxazol-5- |
| | | | ylmethoxy)carbonyl)amino)phenyl)piperidine-1- |
| | | | carboxylate |
| | | | LCMS-ESI (POS.) m/z: 346.1 [M + H-Bu]⁺ |
| | 28.1 | 1,3-oxazol- 5-ylmethanol | 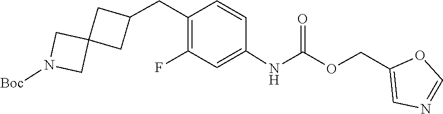 [SMILES: CC(C)(C)OC(=O)N1CC2(CC(Cc3ccc(NC(=O)OCc4cnco4)cc3F)C2)C1] |
| | | | Intermediate 29.6: tert-butyl 6-(2-fluoro-4-(((oxazol- |
| | | | 5-ylmethoxy)carbonyl)amino)benzyl)-2- |
| | | | azaspiro[3.3]heptane-2-carboxylate |
| | | | LCMS-ESI (POS.) m/z: 446.1 [M + H]⁺ |
| | 27.1 | 1,3-oxazol- 5-ylmethanol | 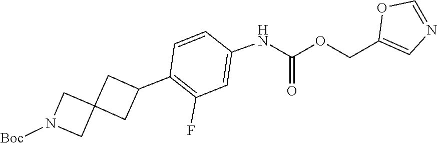 [SMILES: CC(C)(C)OC(=O)N1CC2(CC(c3ccc(NC(=O)OCc4cnco4)cc3F)C2)C1] |
| | | | Intermediate 29.7: tert-butyl 6-(2-fluoro-4-(((oxazol- |
| | | | 5-ylmethoxy)carbonyl)amino)phenyl)-2- |
| | | | azaspiro[3.3]heptane-2-carboxylate |
| | | | LCMS-ESI (POS.) m/z: 376.1 [M + H-Bu]⁺ |
| | 28.8 | 1,3-oxazol- 5-ylmethanol | 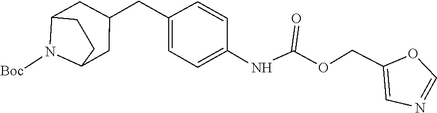 [SMILES: CC(C)(C)OC(=O)N1C2CCC1CC(Cc1ccc(NC(=O)OCc3cnco3)cc1)C2] |
| | | | Intermediate 29.8: tert-butyl 3-(4-(((oxazol-5- |
| | | | ylmethoxy)carbonyl)amino)benzyl)-8- |
| | | | azabicyclo[3.2.1]octane-8-carboxylate |
| | | | LCMS-ESI (POS.) m/z: 386.1 [M + H-Bu]⁺ |
| | tert- butyl 4-(4- amino- 2-fluoro phenyl) piperidine-1- carboxylate | 1,3-oxazol- 5- ylmethanol | 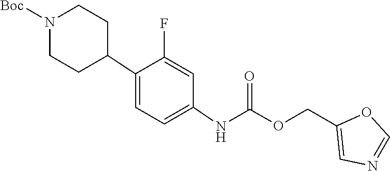 [SMILES: CC(C)(C)OC(=O)N1CCC(c2ccc(NC(=O)OCc3cnco3)cc2F)CC1] |
| | | | Intermediate 29.9: tert-butyl 4-(2-fluoro-4-(((oxazol- |
| | | | 5-ylmethoxy)carbonyl)amino)phenyl)piperidine-1- |
| | | | carboxylate |
| | | | LCMS-ESI (POS.) m/z: 420.1 [M + H]⁺ |
| | 28.5 | 1,3-oxazol- 5-ylmethanol | 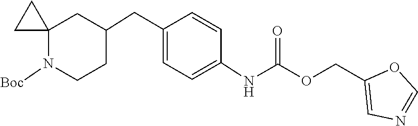 [SMILES: CC(C)(C)OC(=O)N1CCC(Cc2ccc(NC(=O)OCc3cnco3)cc2)CC12CC2] |
| | | | Intermediate 29.10: tert-butyl 7-[(4-{[(1,3-oxazol-5- |
| | | | ylmethoxy)carbonyl]amino}phenyl)methyl]-4- |
| | | | azaspiro[2.5]octane-4-carboxylate |
| | | | LCMS-ESI (POS.) m/z: 420.1 [M + H]⁺ |
| | 28.3 | 1,3-oxazol- 5-ylmethanol | 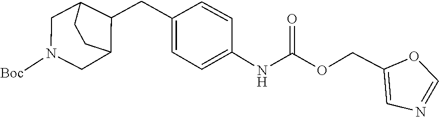 [SMILES: CC(C)(C)OC(=O)N1CC2CCC(C1)C2Cc1ccc(NC(=O)OCc2cnco2)cc1] |
| | | | Intermediate 29.11: tert-butyl 8-(4-(((oxazol-5- |
| | | | ylmethoxy)carbonyl)amino)benzyl)-3- |
| | | | azabicyclo[3.2.1]octane-3-carboxylate |
| | | | LCMS-ESI (POS.) m/z: 442.2 [M + H]⁺ |
| | 28.4 | 1,3-oxazol- 5-ylmethanol | 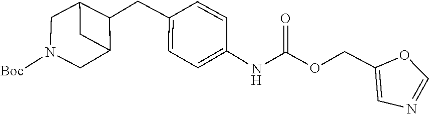 [SMILES: CC(C)(C)OC(=O)N1CC2CC(C1)C2Cc1ccc(NC(=O)OCc2cnco2)cc1] |
| | | | Intermediate 29.12: tert-butyl 6-(4-(((oxazol-5- |
| | | | ylmethoxy)carbonyl)amino)benzyl)-3- |
| | | | azabicyclo[3.1.1]heptane-3-carboxylate |
| | | | LCMS-ESI (POS.) m/z: 428.2 [M + H]⁺ |
| | 28.7 | 1,3-oxazol- 5- ylmethanol | 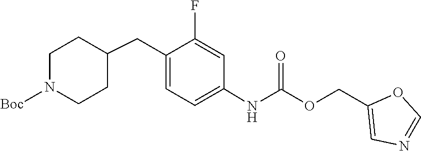 [SMILES: CC(C)(C)OC(=O)N1CCC(Cc2ccc(NC(=O)OCc3cnco3)cc2F)CC1] |
| | | | Intermediate 29.15: tert-butyl 4-(2-fluoro-4-(((oxazol- |
| | | | 5-ylmethoxy)carbonyl)amino)benzyl)piperidine-1- |
| | | | carboxylate. |
| | | | LCMS-ESI (POS.) m/z: 378.1 [M + H-Boc]⁺ |
| | 28.6 | 1,3-oxazol- 5-ylmethanol | 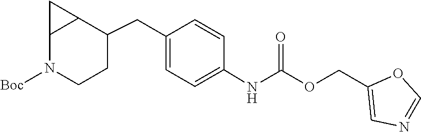 [SMILES: CC(C)(C)OC(=O)N1CCC(Cc2ccc(NC(=O)OCc3cnco3)cc2)C2CC21] |
| | | | Intermediate 29.16: tert-butyl 5-(4-(((oxazol-5- |
| | | | ylmethoxy)carbonyl)amino)benzyl)-2- |
| | | | azabicyclo[4.1.0]heptane-2-carboxylate |
| | | | LCMS-ESI (POS.) m/z: 372.1 [M + H-Bu]⁺ |
| 377 | 28.2 | 1,3-oxazol- 5-ylmethanol | 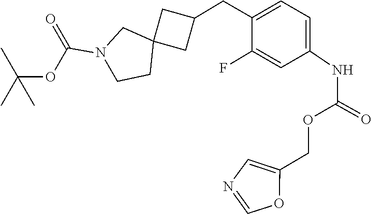 [SMILES: CC(C)(C)OC(=O)N1CCC2(CC(Cc3ccc(NC(=O)OCc4cnco4)cc3F)C2)C1] |
| | | | Compound 377: tert-butyl 2-(2-fluoro-4-(((oxazol-5- |
| | | | ylmethoxy)carbonyl)amino)benzyl)-6- |
| | | | azaspiro[3.4]octane-6-carboxylate |
| | | | LCMS-ES (POS.) m/z: 460.2 [M + H]⁺. |
| | | | ¹H NMR (400 MHz, DMSO-d₆) δ 9.94 (s, 1H), 8.42 (s, |
| | | | 1H), 7.36-7.23 (m, 2H), 7.22-7.05 (m, 2H), 5.22 (s, |
| | | | 2H), 3.23-3.12 (m, 3H), 3.09 (s, 1H), 2.68-2.57 (m, |
| | | | 2H), 2.05-1.83 (m, 2H), 1.84-1.75 (m, 1H), 1.74- |
| | | | 1.62 (m, 4H), 1.37 (s, 9H). |
| | tert- butyl 4-(4- amino benzyl) piperidine-1- carboxylate | thiazol-5- ylmethanol | 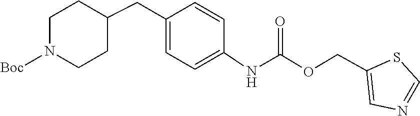 [SMILES: CC(C)(C)OC(=O)N1CCC(Cc2ccc(NC(=O)OCc3cncs3)cc2)CC1] |
| | | | Intermediate 29.17: tert-butyl 4-(4-(((thiazol-5- |
| | | | ylmethoxy)carbonyl)amino)benzyl)piperidine-1- |
| | | | carboxylate |
| | | | LCMS-ESI (POS.) m/z: 376.1 [M + H-Bu]⁺ |

### Method 3: In Situ Preparation Isocyanate Via Curtius Rearrangement

### Example H

### Preparation of oxazol-5-ylmethyl (4-(pyridin-4-ylmethyl)phenyl)carbamate (Compound 201)

### Step 1A: Synthesis of methyl 4-(pyridin-4-ylmethyl)benzoate

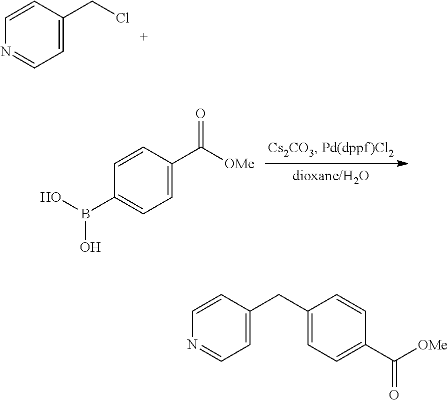

To a solution of 4-(chloromethyl)pyridine hydrochloride (4.92 g, 30 mmol, 1.00 equiv) and 4-(methoxycarbonyl)phenylboronic acid (7.02 g, 39 mmol, 1.30 equiv) in dioxane (70 mL) and H₂O (30 mL) was added cesium carbonate (14.66 g, 45 mmol, 1.50 equiv) and PdCl₂(dppf) (2.20 g, 3 mmol, 0.10 equiv). The reaction mixture was heated at 110° C. in the oil bath for 3 h. The mixture was cooled down to rt and then extracted with EtOAc (100 mL) and H₂O (50 mL). The organic layer was dried, concentrated and purified on silica gel with 50% EtOAc/Hex to afford the desired product methyl 4-(pyridin-4-ylmethyl)benzoate as a white solid (1.70 g, 25%). LRMS (ES) m/z: 228[M+H]⁺.

### Step 1B: Synthesis of methyl 4-((6-methylpyridin-3-yl)methyl)benzoate

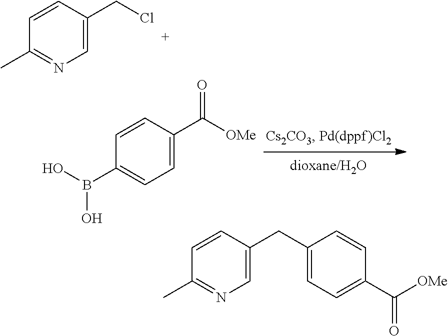

To a solution of 5-(chloromethyl)-2-methylpyridine (3.00 g, 21 mmol, 1.00 equiv) and (4-(methoxycarbonyl)phenyl)boronic acid (5.67 g, 31.50 mmol, 1.50 equiv) in dioxane (80 mL) and H₂O (40 mL) was added cesium carbonate (13.68 g, 42 mmol, 2.00 equiv) and PdCl₂(dppf) (1.54 g, 2.10 mmol, 0.10 equiv). The reaction mixture was heated at 110° C. in the oil bath for 3 h. The mixture was cooled down to rt and then extracted with EtOAc (100 mL) and H₂O (50 mL). The organic layer was dried, concentrated and purified on silica gel with 50% EtOAc/Hex to afford the desired product methyl 4-((6-methylpyridin-3-yl)methyl)benzoate as a white solid (3.87 g, 76%). LRMS (ES) m/z: 242[M+H]⁺. ¹H NMR (400 MHz, Methanol-d₄) δ 8.30 (s, 1H), 7.92 (d, J=7.9 Hz, 2H), 7.54 (d, J=7.8 Hz, 1H), 7.30 (d, J=7.9 Hz, 2H), 7.20 (d, J=8.0 Hz, 1H), 4.00 (s, 2H), 3.86 (s, 3H), 2.48 (s, 3H).

### Step 1C: Synthesis of ethyl 4-((6-methylnicotinamido)methyl)benzoate

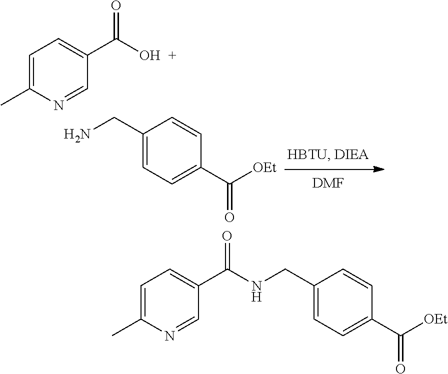

To a solution of 6-methylnicotinic acid (3.43 g, 25 mmol, 1.00 equiv) in DMF (100 mL) was added HBTU (12.32 g, 32.50 mmol, 1.30 equiv), DIEA (6.77 g, 52.50 mmol, 2.10 equiv). The mixture was stirred for 10 min. ethyl 4-(aminomethyl)benzoate (5.38 g, 30 mmol, 1.20 equiv) was added. The reaction mixture was stirred for 1 h. Then the reaction mixture was added EtOAc (120 mL) and brine (150 mL). The organic layer was further washed with s. NaHCO₃(150 mL), dried over MgSO₄, concentrated and purified on silica gel with 100% EtOAc to afford the desired product ethyl 4-((6-methylnicotinamido)methyl)benzoate as a brownish solid (2.98 g, 40%). LRMS (ES) m/z: 299 [M+H]⁺. ¹H NMR (400 MHz, Methanol-d₄) δ 8.91 (d, J=2.6 Hz, 1H), 8.18 (dd, J=8.2, 2.3 Hz, 1H), 8.00 (dd, J=8.3, 2.3 Hz, 2H), 7.53-7.23 (m, 3H), 4.66 (d, J=2.2 Hz, 2H), 4.36 (dd, J=7.2, 2.2 Hz, 2H), 2.61 (d, J=2.1 Hz, 3H), 1.39 (t, J=7.1 Hz, 3H).

### Step 2: Synthesis of 4-(pyridin-4-ylmethyl)benzoic acid

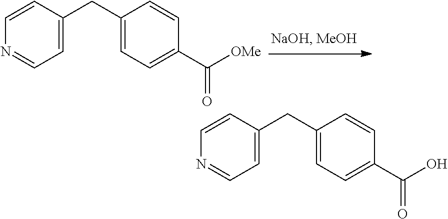

To a solution of methyl 4-(pyridin-4-ylmethyl)benzoate (1.70 g, 7.50 mmol, 1.00 equiv) in MeOH (10 mL) and THF (10 mL) was added NaOH (10 mL, 1.50 M, 2.00 equiv). The reaction mixture was stirred at rt for 16 h. THF and MeOH were removed. The residue was adjusted PH=3-4 by using 2N HCl (7.50 mL). The precipitate was filtered and washed with H₂O, dried over high vacuum to afford the desired product 4-(pyridin-4-ylmethyl)benzoic acid (1.35 g, 84%) as a white solid. LRMS (ES) m/z: 214 [M+H]⁺.

### Step 3: Synthesis of 4-(pyridin-4-ylmethyl)benzoyl azide

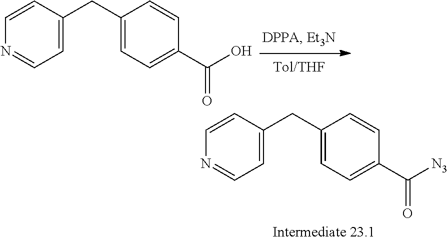

To a mixture of 4-(pyridin-4-ylmethyl)benzoic acid (1.75 g, 8.21 mmol, 1.00 equiv) in toluene/THF (20 mL/20 mL) was added Et₃N (0.83 g, 8.21 mmol, 1.00 equiv), followed by diphenylphosphoryl azide (2.26 g, 8.21 mmol, 1.00 equiv) The reaction mixture was stirred at r.t. for 16 h. The reaction mixture was concentrated and purified by silica gel chromatography (0-100% ethyl acetate/hexanes) to afford 4-(pyridin-4-ylmethyl)benzoyl azide as a white solid (1.72 g, 88%). LRMS (ES) m/z: 239 [M+H]⁺.

### Step 4: Synthesis of thiazol-5-ylmethyl (4-(pyridin-4-ylmethyl)phenyl)carbamate

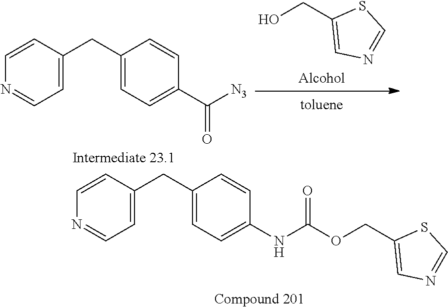

To a mixture of 4-(pyridin-4-ylmethyl)benzoyl azide (29 mg, 0.12 mmol, 1.0 equiv) in toluene (1 mL) was added 1,3-thiazol-5-ylmethanol (28 mg, 0.24 mmol, 2.00 equiv). The reaction mixture was heated at 110° C. for 1 h. The solvent was removed and the residue was purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford the desired product thiazol-5-ylmethyl (4-(pyridin-4-ylmethyl)phenyl)carbamate as a white solid (35 mg, 90%). LRMS (ES) m/z: 326 [M+H]⁺. ¹H NMR (400 MHz, Methanol-d₄) δ 9.02 (s, 1H), 8.42 (d, J=5.2 Hz, 2H), 7.96 (s, 1H), 7.41 (d, J=8.1 Hz, 2H), 7.29 (d, J=5.3 Hz, 2H), 7.17 (d, J=8.1 Hz, 2H), 5.43 (s, 2H), 4.00 (s, 2H).

Compounds in the following table were prepared in a similar manner as compound 201, using the starting materials and alcohols as listed.

| Co | Starting | | |
|---|---|---|---|
| # | Material | Alcohol | Structure, Name and Data |
| 153 | 4- (chloromethyl) pyridine | oxazol-5- ylmethanol | 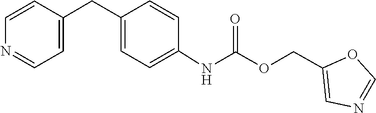 [SMILES: O=C(Nc1ccc(Cc2ccncc2)cc1)OCc1cnco1] |
| | | | Compound 153: oxazol-5-ylmethyl (4-(pyridin-4- |
| | | | ylmethyl)phenyl)carbamate. LRMS (ES) m/z: 310 |
| | | | [M + H]⁺. ¹H NMR (400 MHz, Methanol-d₄) δ 8.42 (s, |
| | | | 2H), 8.23 (s, 1H), 7.40 (d, J = 8.1 Hz, 2H), 7.34-7.21 |
| | | | (m, 3H), 7.16 (d, J = 8.1 Hz, 2H), 5.24 (s, 2H), 3.99 (s, |
| | | | 2H). |
| 355 | 4- (chloromethyl) pyridine | (1-methyl- 1H-pyrazol- 4- yl)methanol | 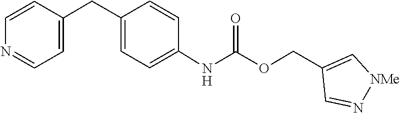 [SMILES: Cn1cc(COC(=O)Nc2ccc(Cc3ccncc3)cc2)cn1] |
| | | | Compound 355: (1-methyl-1H-pyrazol-4-yl)methyl (4- |
| | | | (pyridin-4-ylmethyl)phenyl)carbamate. LRMS (ES) |
| | | | m/z: 323 [M + H]⁺. ¹H NMR (400 MHz, Methanol-d₄) δ |
| | | | 8.41 (d, J = 5.1 Hz, 2H), 7.67 (s, 1H), 7.54 (s, 1H), 7.39 |
| | | | (d, J = 8.1 Hz, 2H), 7.27 (d, J = 5.1 Hz, 2H), 7.14 (d, J = |
| | | | 8.1 Hz, 2H), 5.06 (s, 2H), 3.97 (s, 2H), 3.87 (s, 3H). |
| 333 | 4- (chloromethyl) pyridine | isoxazol-4- ylmethanol | 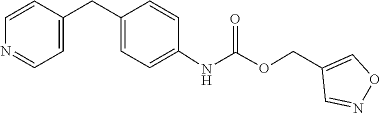 [SMILES: O=C(Nc1ccc(Cc2ccncc2)cc1)OCc1cnoc1] |
| | | | Compound 333: isoxazol-4-ylmethyl (4-(pyridin-4- |
| | | | ylmethyl)phenyl)carbamate. LRMS (ES) m/z: 310 |
| | | | [M + H]⁺. |
| | | | ¹H NMR (400 MHz, Methanol-d₄) δ 8.79 (s, 1H), 8.53 (s, |
| | | | 1H), 8.41 (d, J = 5.1 Hz, 2H), 7.40 (d, J = 8.1 Hz, 2H), |
| | | | 7.28 (d, J = 5.1 Hz, 2H), 7.17 (d, J = 8.1 Hz, 2H), 5.12 (s, |
| | | | 2H), 3.99 (s, 2H). |
| 219 | 4- (chloromethyl) pyridine | isothiazol-5- ylmethanol | 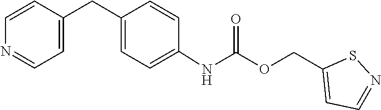 [SMILES: O=C(Nc1ccc(Cc2ccncc2)cc1)OCc1ccns1] |
| | | | Compound 219: isothiazol-5-ylmethyl (4-(pyridin-4- |
| | | | ylmethyl)phenyl)carbamate LRMS (ES) m/z: 326 |
| | | | [M + H]⁺. ¹H NMR (400 MHz, Methanol-d₄) δ 8.48- |
| | | | 8.32 (m, 3H), 7.43 (d, J = 8.1 Hz, 2H), 7.36 (s, 1H), 7.27 |
| | | | (d, J = 5.1 Hz, 2H), 7.17 (d, J = 8.2 Hz, 2H), 5.50 (s, |
| | | | 2H), 3.99 (s, 2H). |
| 299 | 4- (chloromethyl) pyridine | isothiazol-4- ylmethanol | 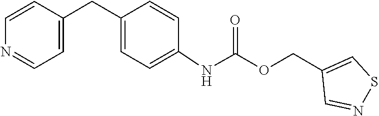 [SMILES: O=C(Nc1ccc(Cc2ccncc2)cc1)OCc1cnsc1] |
| | | | Compound 299: isothiazol-4-ylmethyl (4-(pyridin-4- |
| | | | ylmethyl)phenyl)carbamate. LRMS (ES) m/z: 326 |
| | | | [M + H]⁺. ¹H NMR (400 MHz, Methanol-d₄) δ 8.89 (s, |
| | | | 1H), 8.59 (s, 1H), 8.52-8.25 (m, 2H), 7.41 (d, J = 8.1 |
| | | | Hz, 2H), 7.25 (d, J = 5.1 Hz, 2H), 7.15 (d, J = 8.2 Hz, |
| | | | 2H), 5.29 (s, 2H), 3.97 (s, 2H). |
| 169 | 4- (chloromethyl) pyridine | pyridin-4- ylmethanol | 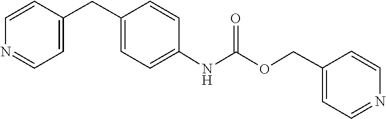 [SMILES: O=C(Nc1ccc(Cc2ccncc2)cc1)OCc1ccncc1] |
| | | | Compound 169: pyridin-4-ylmethyl (4-(pyridin-4- |
| | | | ylmethyl)phenyl)carbamate. LRMS (ES) m/z: 320 |
| | | | [M + H]⁺. ¹H NMR (400 MHz, Methanol-d₄) δ 8.54 (d, J = |
| | | | 5.0 Hz, 2H), 8.42 (d, J = 5.1 Hz, 2H), 7.52-7.38(m, |
| | | | 4H), 7.29 (d, J = 5.0 Hz, 2H), 7.18 (d, J = 8.0 Hz, 2H), |
| | | | 5.27 (s, 2H), 4.00 (s, 2H). |
| 349 | 4- (chloromethyl) pyridine | pyrimidin- 4- ylmethanol | 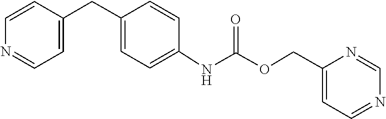 [SMILES: O=C(Nc1ccc(Cc2ccncc2)cc1)OCc1ccncn1] |
| | | | Compound 349: pyrimidin-4-ylmethyl (4-(pyridin-4- |
| | | | ylmethyl)phenyl)carbamate. LRMS (ES) m/z: 321 |
| | | | [M + H]⁺. ¹H NMR (400 MHz, Methanol-d₄) δ 9.13 (s, |
| | | | 1H), 8.79 (d, J = 5.2 Hz, 1H), 8.42 (d, J = 5.2 Hz, 2H), |
| | | | 7.59 (d, J = 5.2 Hz, 1H), 7.44 (d, J = 8.1 Hz, 2H), 7.29 (d, |
| | | | J = 5.1 Hz, 2H), 7.18 (d, J = 8.1 Hz, 2H), 5.28 (s, 2H), 4.00 |
| | | | (s, 2H). |
| 326 | 4- (chloromethyl) pyridine | pyridazin-4- ylmethanol | 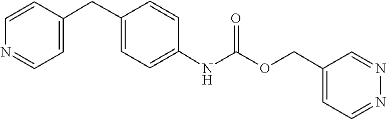 [SMILES: O=C(Nc1ccc(Cc2ccncc2)cc1)OCc1ccnnc1] |
| | | | Compound 326: pyridazin-4-ylmethyl (4-(pyridin-4- |
| | | | ylmethyl)phenyl)carbamate. LRMS (ES) m/z: 321 |
| | | | [M + H]⁺. ¹H NMR (400 MHz, Methanol-d₄) δ 9.28 (s, 1H), |
| | | | 9.20 (d, J = 5.3 Hz, 1H), 8.42 (d, J = 5.1 Hz, 2H), 7.84- |
| | | | 7.71 (m, 1H), 7.43 (d, J = 8.1 Hz, 2H), 7.29 (d, J = 5.1 Hz, |
| | | | 2H), 7.19 (d, J = 8.1 Hz, 2H), 5.32 (s, 2H), 4.00 (s, 2H). |
| 106 | 4- (chloromethyl) pyridine | 1,4- phenylenedi- methanol | 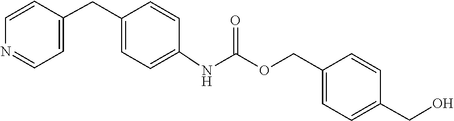 [SMILES: O=C(Nc1ccc(Cc2ccncc2)cc1)OCc1ccc(CO)cc1] |
| | | | Compound 106: 4-(hydroxymethyl)benzyl (4- |
| | | | (pyridin-4-ylmethyl)phenyl)carbamate. LRMS (ES) |
| | | | m/z: 349 [M + H]⁺. ¹H NMR (400 MHz, Methanol-d₄) δ |
| | | | 8.40 (d, J = 5.0 Hz, 2H), 7.47-7.31 (m, 6H), 7.27 (d, J = |
| | | | 5.0 Hz, 2H), 7.14 (d, J = 8.2 Hz, 2H), 5.17 (s, 2H), 4.61 |
| | | | (s, 2H), 3.96 (s, 2H). |
| 196 | 4- (chloromethyl) pyridine | (4- (difluoromethyl) phenyl) methanol | 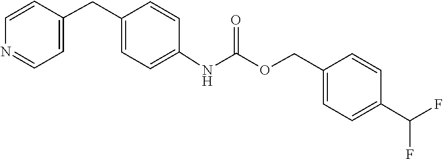 [SMILES: O=C(Nc1ccc(Cc2ccncc2)cc1)OCc1ccc(C(F)F)cc1] |
| | | | Compound 196: 4-(difluoromethyl)benzyl (4-(pyridin- |
| | | | 4-ylmethyl)phenyl)carbamate. LRMS (ES) m/z: 369 |
| | | | [M + H]⁺. ¹H NMR (400 MHz, Methanol-d₄) δ 8.47-8.24 |
| | | | (m, 2H), 7.68-7.45 (m, 4H), 7.42 (d, J = 8.2 Hz, 2H), |
| | | | 7.35-7.25 (m, 2H), 7.25-7.09 (m, 2H), 6.76 (t, J = 56 |
| | | | Hz, 1H), 5.23 (s, 2H), 3.98 (s, 2H). |
| 14 | 4- (chloromethyl) pyridine | 4- (hydroxymethyl) benzamide | 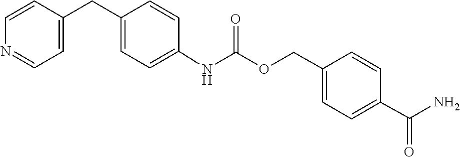 [SMILES: NC(=O)c1ccc(COC(=O)Nc2ccc(Cc3ccncc3)cc2)cc1] |
| | | | Compound 14: 4-carbamoylbenzyl (4-(pyridin-4- |
| | | | ylmethyl)phenyl)carbamate. LRMS (ES) m/z: 362 |
| | | | [M + H]⁺. ¹H NMR (400 MHz, Methanol-d₄) δ 8.42 (d, J = |
| | | | 5.1 Hz, 2H), 7.90 (d, J = 8.1 Hz, 2H), 7.53 (d, J = 8.1 Hz, |
| | | | 2H), 7.42 (d, J = 8.1 Hz, 2H), 7.30 (d, J = 5.2 Hz, 2H), |
| | | | 7.17 (d, J = 8.3 Hz, 2H), 5.25 (s, 2H), 4.00 (s, 2H). |
| 99 | 4- (chloromethyl) pyridine | (1H- pyrazol-4- yl)methanol | 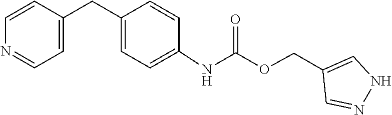 [SMILES: O=C(Nc1ccc(Cc2ccncc2)cc1)OCc1cn[nH]c1] |
| | | | Compound 99: (1H-pyrazol-4-yl)methyl (4-(pyridin-4- |
| | | | ylmethyl)phenyl)carbamate LRMS (ES) m/z: 309 |
| | | | [M + H]⁺. ¹H NMR (400 MHz, Methanol-d₄) δ 8.51- |
| | | | 8.29 (m, 2H), 7.70 (s, 2H), 7.40 (d, J = 8.1 Hz, 2H), 7.34- |
| | | | 7.23 (m, 2H), 7.16 (d, J = 8.3 Hz, 2H), 5.12 (s, 2H), |
| | | | 3.99 (s, 2H). |
| 51 | 5- (chloromethyl)- 2-methylpyridine | oxazol-5- ylmethanol | 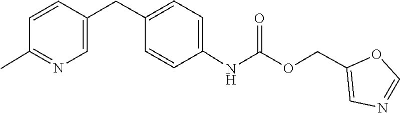 [SMILES: Cc1ccc(Cc2ccc(NC(=O)OCc3cnco3)cc2)cn1] |
| | | | Compound 51: oxazol-5-ylmethyl (4-((6- |
| | | | methylpyridin-3-yl)methyl)phenyl)carbamate. LRMS |
| | | | (ES) m/z: 324 [M + H]⁺. ¹H NMR (400 MHz, Methanol- |
| | | | d₄) δ 8.29 (s, 1H), 8.23 (s, 1H), 7.60 (dd, J = 8.1, 2.0 Hz, |
| | | | 1H), 7.38 (d, J = 8.1 Hz, 2H), 7.26 (d, J = 11.2 Hz, 2H), |
| | | | 7.14 (d, J = 8.1 Hz, 2H), 5.23 (s, 2H), 3.93 (s, 2H), 2.51 |
| | | | (s, 3H). |
| 114 | 5- (chloromethyl)- 2-methylpyridine | thiazol-5- ylmethanol | 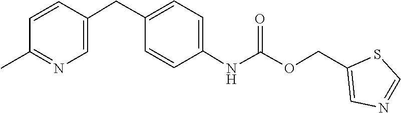 [SMILES: Cc1ccc(Cc2ccc(NC(=O)OCc3cncs3)cc2)cn1] |
| | | | Compound 114: thiazol-5-ylmethyl (4-((6- |
| | | | methylpyridin-3-yl)methyl)phenyl)carbamate. LRMS |
| | | | (ES) m/z: 340 [M + H] ¹H NMR (400 MHz, Methanol-d₄) |
| | | | δ 9.01 (s, 1H), 8.29 (s, 1H), 7.95 (s, 1H), 7.61 (d, J = 8.1 |
| | | | Hz, 1H), 7.39 (d, J = 8.1 Hz, 2H), 7.26 (d, J = 8.0 Hz, |
| | | | 1H), 7.14 (d, J = 8.1 Hz, 2H), 5.41 (s, 2H), 3.93 (s, 2H), |
| | | | 2.51 (s, 3H). |
| 186 | 5- (chloromethyl)- 2-methylpyridine | pyridin-4- ylmethanol | 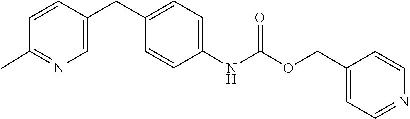 [SMILES: Cc1ccc(Cc2ccc(NC(=O)OCc3ccncc3)cc2)cn1] |
| | | | Compound 186: pyridin-4-ylmethyl (4-((6- |
| | | | methylpyridin-3-yl)methyl)phenyl)carbamate. LRMS |
| | | | (ES) m/z: 334 [M + H]⁺. ¹H NMR (400 MHz, Methanol- |
| | | | d₄) δ 8.53 (d, J = 5.0 Hz, 2H), 8.30 (s, 1H), 7.62 (dd, J = |
| | | | 8.1, 2.0 Hz, 1H), 7.47 (d, J = 5.1 Hz, 2H), 7.42 (d, J = 8.2 |
| | | | Hz, 2H), 7.27 (d, J = 8.0 Hz, 1H), 7.15 (d, J = 8.1 Hz, |
| | | | 2H), 5.26 (s, 2H), 3.94 (s, 2H), 2.52 (s, 3H). |
| 331 | 5- (chloromethyl)- 2-methylpyridine | (4- fluorophenyl) methanol | 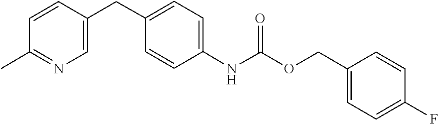 [SMILES: Cc1ccc(Cc2ccc(NC(=O)OCc3ccc(F)cc3)cc2)cn1] |
| | | | Compound 331: 4-fluorobenzyl (4-((6-methylpyridin- |
| | | | 3-yl)methyl)phenyl)carbamate. LRMS (ES) m/z: 351 |
| | | | [M + H]⁺. ¹H NMR (400 MHz, Methanol-d₄) δ 8.26 (s, |
| | | | 1H), 7.54 (dd, J = 8.1, 2.0 Hz, 1H), 7.45 (dd, J = 8.1, 5.6 |
| | | | Hz, 2H), 7.38 (d, J = 8.1 Hz, 2H), 7.21 (d, J = 8.0 Hz, |
| | | | 1H), 7.18-6.99 (m, 4H), 5.15 (s, 2H), 3.92 (s, 2H), 2.49 |
| | | | (s, 3H). |
| 328 | 5- (chloromethyl)- 2-methylpyridine | (4- (difluoromethyl) phenyl) methanol | 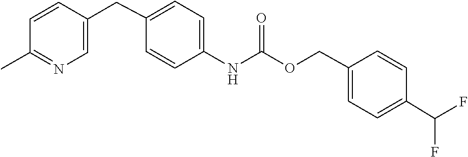 [SMILES: Cc1ccc(Cc2ccc(NC(=O)OCc3ccc(C(F)F)cc3)cc2)cn1] |
| | | | Compound 328: (4-(difluoro-13-methyl)phenyl)methyl |
| | | | (4-((6-methylpyridin-3-yl)methyl)phenyl)carbamate |
| | | | LRMS (ES) m/z: 383 [M + H]⁺. ¹H NMR (400 MHz, |
| | | | Methanol-d₄) δ 8.27 (s, 1H), 7.60-7.50 (m, 5H), 7.39 (d, |
| | | | J = 8.1 Hz, 2H), 7.23 (d, J = 8.0 Hz, 1H), 7.17-7.06 (m, |
| | | | 2H), 6.77 (d, J = 56 Hz, 1H), 5.23 (s, 2H), 3.93 (s, 2H), |
| | | | 2.50 (s, 3H). |
| 65 | 5- (chloromethyl)- 2-methylpyridine | (1H- pyrazol-4- yl)methanol | 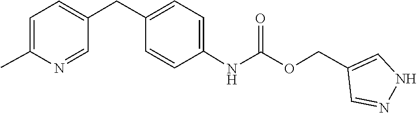 [SMILES: Cc1ccc(Cc2ccc(NC(=O)OCc3cn[nH]c3)cc2)cn1] |
| | | | Compound 65: (1H-pyrazol-4-yl)methyl (4-((6- |
| | | | methylpyridin-3-yl)methyl)phenyl)carbamate. LRMS |
| | | | (ES) m/z: 323 [M + H]⁺. ¹H NMR (400 MHz, Methanol- |
| | | | d₄) δ 8.31 (s, 1H), 7.70 (s, 2H), 7.66 (dd, J = 8.1, 2.0 Hz, |
| | | | 1H), 7.38 (d, J = 8.1 Hz, 2H), 7.30 (d, J = 8.1 Hz, 1H), |
| | | | 7.13 (d, J = 8.1 Hz, 2H), 5.11 (s, 2H), 3.94 (s, 2H), 2.53 |
| | | | (s, 3H). |
| 292 | 5- (chloromethyl)- 2-methylpyridine | (4- chlorophenyl) methanol | 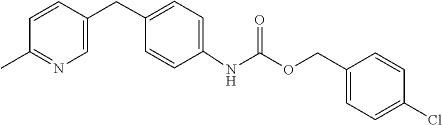 [SMILES: Cc1ccc(Cc2ccc(NC(=O)OCc3ccc(Cl)cc3)cc2)cn1] |
| | | | Compound 292: 4-chlorobenzyl (4-((6-methylpyridin- |
| | | | 3-yl)methyl)phenyl)carbamate. LRMS (ES) m/z: 367 |
| | | | [M + H]⁺. ¹H NMR (400 MHz, Methanol-d₄) δ 8.27 (s, 1H), |
| | | | 7.55 (d, J = 8.2 Hz, 1H), 7.46-7.30 (m, 6H), 7.23 (d, J = |
| | | | 8.1 Hz, 1H), 7.14 (d, J = 8.1 Hz, 2H), 5.16 (s, 2H), 3.93 (s, |
| | | | 2H), 2.50 (s, 3H). |
| 25 | 6- methylnicotinic acid | oxazol-5- ylmethanol | 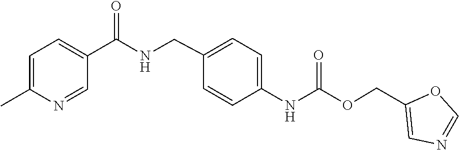 [SMILES: Cc1ccc(C(=O)NCc2ccc(NC(=O)OCc3cnco3)cc2)cn1] |
| | | | Compound 25: oxazol-5-ylmethyl (4-((6- |
| | | | methylnicotinamido)methyl)phenyl)carbamate. LRMS |
| | | | (ES) m/z: 367 [M + H]⁺. ¹H NMR (400 MHz, Methanol- |
| | | | d₄) δ 8.88 (s, 1H), 8.23 (s, 1H), 8.15 (dd, J = 8.2, 2.2 Hz, |
| | | | 1H), 7.42 (t, J = 7.1 Hz, 3H), 7.38-7.28 (m, 2H), 7.25 (s, |
| | | | 1H), 5.25 (s, 2H), 4.54 (s, 2H), 2.60 (s, 3H). |
| 66 | 6- methylnicotinic acid | thiazol-5- ylmethanol | 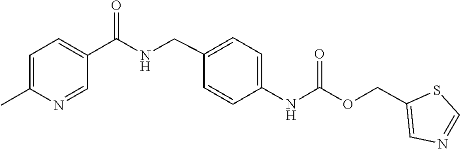 [SMILES: Cc1ccc(C(=O)NCc2ccc(NC(=O)OCc3cncs3)cc2)cn1] |
| | | | Compound 66: thiazol-5-ylmethyl (4-((6- |
| | | | methylnicotinamido)methyl)phenyl)carbamate. |
| | | | LRMS (ES) m/z: 383 [M + H]⁺. ¹H NMR (400 MHz, |
| | | | Methanol-d₄) δ (s 8 9.03 (s, 1H), 8.89 (s, 1H), 8.16 (d, J = |
| | | | 8.3 Hz, 1H), 7.96 (s, 1H), 7.42 (q, J = 6.7 Hz, 3H), 7.31 |
| | | | (d, J = 8.1 Hz, 2H), 5.43 (s, 2H), 4.54 (s, 2H), 2.61 (s, 3H). |
| 227 | 6- methylnicotinic acid | (4- fluorophenyl) methanol | 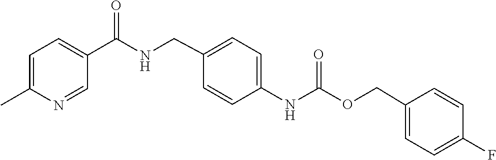 [SMILES: Cc1ccc(C(=O)NCc2ccc(NC(=O)OCc3ccc(F)cc3)cc2)cn1] |
| | | | Compound 227: 4-fluorobenzyl (4-((6- |
| | | | methylnicotinamido)methyl)phenyl)carbamate. |
| | | | LRMS (ES) m/z: [M + H]⁺. ¹H NMR (400 MHz, |
| | | | Methanol-d₄) δ 8.88 (s, 1H), 8.16 (d, J = 8.2 Hz, 1H), |
| | | | 8.12 (s, 1H), 7.55-7.37 (m, 5H), 7.30 (d, J = 8.3 Hz, |
| | | | 2H), 7.11 (t, J = 8.6 Hz, 2H), 5.16 (s, 2H), 4.54 (s, 2H), |
| | | | 2.60 (s, 3H). |
| 53 | 6- methylnicotinic acid | (4- methoxyphenyl) methanol | 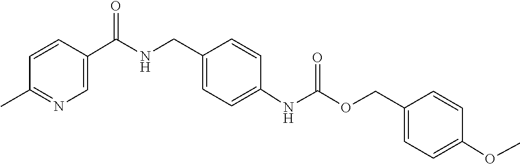 |
| | | | Compound 53: (4-methoxyphenyl)methyl N-(4-{[(6- |
| | | | methylpyridin-3- |
| | | | yl)formamido]methyl}phenyl)carbamate. LCMS-ESI |
| | | | (POS.) m/z: 406.0 [M + H]⁺. ¹H NMR (300 MHz, DMSO- |
| | | | d₆) δ 9.65 (s, 1H), 9.06 (s, 1H), 8.92 (d, J = 2.8 Hz, 1H), |
| | | | 8.16-8.07 (m, 1H), 7.46-7.31 (m, 5H), 7.28-7.19 (m, |
| | | | 2H), 7.00-6.90 (m, 2H), 5.06 (d, J = 2.0 Hz, 2H), 4.42 (d, |
| | | | J = 6.0 Hz, 2H), 3.76 (d, J = 1.9 Hz, 3H), 2.50 (s, 3H). |
| 133 | 6- methylnicotinic acid | (4- (difluoromethyl) phenyl) methanol | 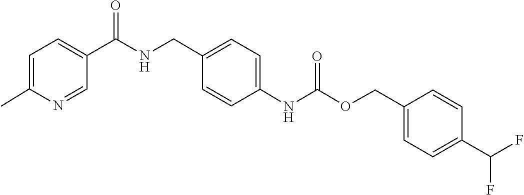 [SMILES: Cc1ccc(C(=O)NCc2ccc(NC(=O)OCc3ccc(C(F)F)cc3)cc2)cn1] |
| | | | Compound 133: (4-(difluoro-13-methyl)phenyl)methyl |
| | | | (4-((6-methylnicotinamido)methyl)phenyl)carbamate |
| | | | LRMS (ES) m/z: 426 [M + H]⁺. ¹H NMR (400 MHz, |
| | | | Methanol-d₄) δ 8.89 (s, 1H), 8.21-8.08 (m, 1H), 7.56 (m, |
| | | | 4H), 7.43 (t, J = 9.0 Hz, 3H), 7.31 (d, J = 8.1 Hz, 2H), 6.77 |
| | | | (t, J = 56 Hz, 1H), 5.25 (s, 2H), 4.54 (s, 2H), 2.61 (s, 3H). |
| 275 | 6- methylnicotinic acid | (4- (difluoromethoxy) phenyl) methanol | 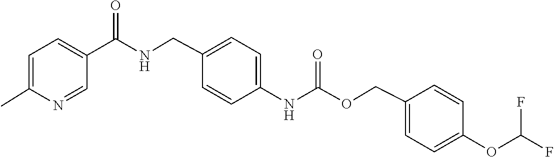 [SMILES: Cc1ccc(C(=O)NCc2ccc(NC(=O)OCc3ccc(OC(F)F)cc3)cc2)cn1] |
| | | | Compound 275: (4-(difluoro-13-methyl)phenyl)methyl |
| | | | (4-((6-methylnicotinamido)methyl)phenyl)carbamate |
| | | | LRMS (ES) m/z: 442 [M + H]⁺. ¹H NMR (400 MHz, |
| | | | Methanol-d₄) δ 8.89 (s, 1H), 8.21-8.08 (m, 1H), 7.56 (m, |
| | | | 5H), 7.43 (t, J = 9.0 Hz, 2H), 7.31 (d, J = 8.1 Hz, 2H), 6.77 |
| | | | (t, J = 56 Hz, 1H), 5.25 (s, 2H), 4.54 (s, 2H), 2.61 (s, 3H). |
| 9 | 6- methylnicotinic acid | (1H- pyrazol-4- yl)methanol | 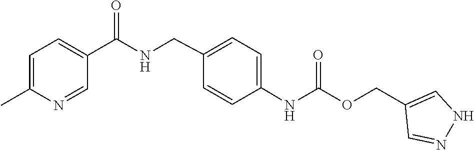 [SMILES: Cc1ccc(C(=O)NCc2ccc(NC(=O)OCc3cn[nH]c3)cc2)cn1] |
| | | | Compound 9: (1H-pyrazol-4-yl)methyl (4-((6- |
| | | | methylnicotinamido)methyl)phenyl)carbamate. |
| | | | LRMS (ES) m/z: 366 [M + H]⁺. ¹H NMR (400 MHz, |
| | | | Methanol-d₄) δ 8.88 (s, 1H), 8.15 (dd, J = 8.1, 2.3 Hz, |
| | | | 1H), 7.70 (s, 2H), 7.41 (t, J = 6.3 Hz, 3H), 7.29 (d, J = |
| | | | 8.1 Hz, 2H), 5.12 (s, 2H), 4.54 (d, J = 5.0 Hz, 2H), 2.60 |
| | | | (d, J = 2.1 Hz, 3H). |
| 37 | 6- methylnicotinic acid | pyridin-4- ylmethanol | 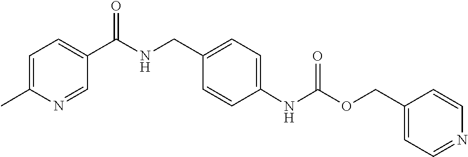 [SMILES: Cc1ccc(C(=O)NCc2ccc(NC(=O)OCc3ccncc3)cc2)cn1] |
| | | | Compound 37: pyridin-4-ylmethyl (4-((6- |
| | | | methylnicotinamido)methyl)phenyl)carbamate. |
| | | | LRMS (ES) m/z: 377 [M + H]⁺. ¹H NMR (400 MHz, |
| | | | Methanol-d₄) δ 8.9 (s, 1H), 8.54 (d, J = 5.0 Hz, 2H), 8.16 |
| | | | (dd, J = 8.1, 2.0 Hz, 1H), 7.58-7.37 (m, 5H), 7.31 (d, J = |
| | | | 8.2 Hz, 2H), 5.26 (s, 2H), 4.54 (s, 2H), 2.60 (s, 3H). |

### Method 4: Synthesis of Carbamates by Phenol Replacement

### Example I

### Preparation of oxazol-5-ylmethyl (R)-(4-((2-(pyridin-3-yl)pyrrolidin-1-yl)methyl)phenyl)carbamate (Compound 6)

### Step 1: Synthesis of phenyl N-(4-formylphenyl)carbamate

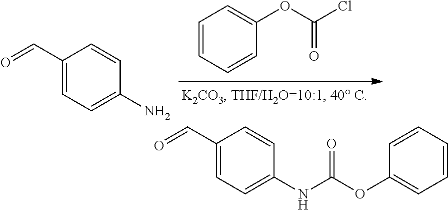

To a solution of 4aminobenzaldehyde (5 g, 4.8 mmol, 1.00 eq.) in THF (100 mL and H₂O (10 mL) at −5° C. was added K₂CO₃ (11.4 g, 82.4 mmol, 2.00 eq.) and phenyl chloroformate (9.7 g, 61.96 mmol, 1.50 eq.) dropwise over 15 min. The resulting mixture was stirred at 40° C. for 1 h. The resulting mixture was cooled to r.t., added water (50 mL), extracted twice with EtOAc (40 mL). The combined organic layers were washed with brine (40 mL), dried over anhydrous Na₂SO₄, concentrated under reduced pressure, purified by silica gel column chromatography, and eluted with hexane/EtOAc (10:1) to afford phenyl N-(4-formylphenyl)carbamate (10.9 g, 93.1%) as a yellow solid. LRMS (ES) m/z: 242 [M+H]⁺.

### Step 2: Synthesis of phenyl N-(4-{[(2R)-2-(pyridin-3-yl)pyrrolidin-1-yl]methyl}phenyl)carbamate

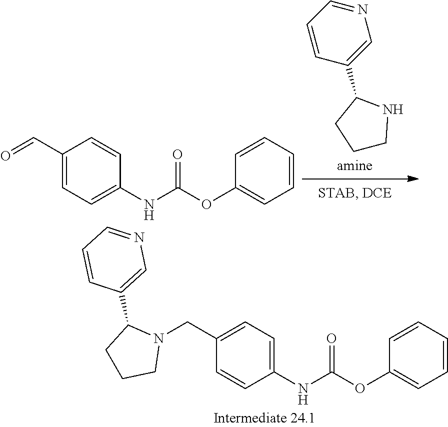

To a stirred solution of phenyl N-(4-formylphenyl)carbamate (720 mg, 2.99 mmol, 1.1 eq.) and (R)-3-(pyrrolidin-2-yl)pyridine (402 mg, 2.71 mmol, 1 eq.) in DCE (8 mL) was added STAB (1150 mg, 5.43 mmol, 2 eq.). The resulting mixture was stirred at r.t. for 2 h. The resulting mixture was added water (10 mL), extracted twice with CH₂Cl₂ (20 mL). The combined organic layers were washed with brine (20 mL), dried over anhydrous Na₂SO₄, concentrated under reduced pressure, purified by silica gel column chromatography, eluted with hexane/EtOAc (5:1) to afford phenyl N-(4-{[(2R)-2-(pyridin-3-yl)pyrrolidin-1-yl]methyl}phenyl)carbamate (700 mg, 67.0%) as a yellow solid. LRMS (ES) m/z: 374 [M+H]⁺.

### Step 3: Synthesis of oxazol-5-ylmethyl (R)-(4-((2-(pyridin-3-yl)pyrrolidin-1-yl)methyl)phenyl)carbamate

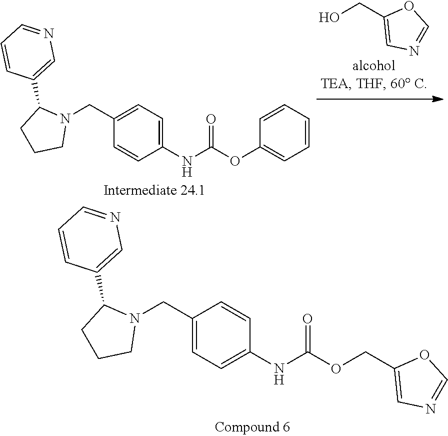 [SMILES: O=C(Nc1ccc(CN2CCC[C@@H]2c2cccnc2)cc1)Oc1ccccc1 ; OCc1cnco1 ; O=C(Nc1ccc(CN2CCC[C@@H]2c2cccnc2)cc1)OCc1cnco1]

To a stirred solution of phenyl N-(4-{[(2R)-2-(pyridin-3-yl)pyrrolidin-1-yl]methyl}phenyl)carbamate (70 mg, 0.19 mmol, 1 equiv) in THF (1 mL) was added 1,3-oxazol-5-ylmethanol (22 mg, 0.22 mmol, 1.18 equiv) and TEA (56 mg, 0.55 mmol, 2.95 equiv) at room temperature. The resulting mixture was stirred at 60° C. overnight. The mixture was allowed to cool down to room temperature. The crude product was purified by Prep-HPLC with the following conditions (2 #SHIMADZU (HPLC-01)): Column, XBridge Prep OBD C18 Column, 30*150 mm, 5 μm; mobile phase, Water (10 mmol/L NH4HCO₃+0.1% NH3·H₂O) and ACN (33% ACN up to 47% in 8 min); Detector, uv 254 nm to afford 5.1 mg of 1,3-oxazol-5-ylmethyl N-(4-{[(2R)-2-(pyridin-3-yl)pyrrolidin-1-yl]methyl}phenyl)carbamate as a white solid. LRMS (ES) m/z: 379 [M+H]⁺. 1H NMR (300 MHz, DMSO-d6) δ 9.75 (s, 1H), 8.59 (d, J=2.2 Hz, 1H), 8.50-8.39 (m, 2H), 7.84 (dt, J=7.9, 2.0 Hz, 1H), 7.43-7.33 (m, 3H), 7.30 (s, 1H), 7.14 (d, J=8.3 Hz, 2H), 5.20 (s, 2H), 3.58 (d, J=13.1 Hz, 1H), 3.43 (t, J=8.1 Hz, 1H), 3.05 (d, J=13.1 Hz, 1H), 2.96 (td, J=9.2, 8.3, 3.0 Hz, 1H), 2.20 (q, J=8.7 Hz, 2H), 1.77 (d, J=9.9 Hz, 2H), 1.60 (dq, J=18.0, 10.1, 8.1 Hz, 1H).

Compounds in the following table were prepared in a similar manner as Compound 6, using the amine and alcohol as listed.

| Co# | Amine | Alcohol | Structure, Name and Data |
|---|---|---|---|
| 1 | (R)-3- (pyrrolidin- 2-yl) pyridine | (4- methoxyphenyl) methanol | 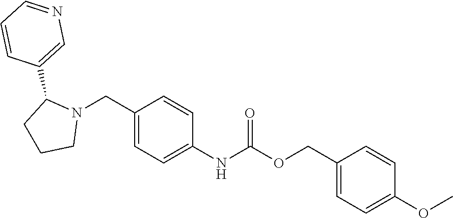 [SMILES: COc1ccc(COC(=O)Nc2ccc(CN3CCC[C@@H]3c3cccnc3)cc2)cc1] |
| | | | Compound 1: (4-methoxyphenyl)methyl N-(4- |
| | | | {[(2R)-2-(pyridin-3-yl)pyrrolidin-1- |
| | | | yl]methyl}phenyl)carbamate. LCMS-ESI (POS.) |
| | | | m/z: 418.0 [M + H]⁺. ¹H NMR (300 MHz, DMSO-d6) |
| | | | δ 9.64 (s, 1H), 8.59 (dd, J = 2.3, 0.8 Hz, 1H), 8.46 (dd, |
| | | | J = 4.7, 1.7 Hz, 1H), 7.84 (dt, J = 7.8, 2.0 Hz, 1H), 7.43- |
| | | | 7.30 (m, 5H), 7.12 (d, J = 8.4 Hz, 2H), 7.00-6.90 |
| | | | (m, 2H), 5.05 (s, 2H), 3.75 (s, 3H), 3.58 (d, J = 13.0 |
| | | | Hz, 1H), 3.43 (t, J = 8.1 Hz, 1H), 3.04 (d, J = 13.1 Hz, |
| | | | 1H), 3.00-2.89 (m, 1H), 2.18 (t, J = 8.8 Hz, 2H), 1.76 |
| | | | (d, J = 9.1 Hz, 2H), 1.58 (d, J = 9.0 Hz, 1H). |
| 3 | (R)-3- (pyrrolidin- 2-yl) pyridine | pyridin-4- ylmethanol | 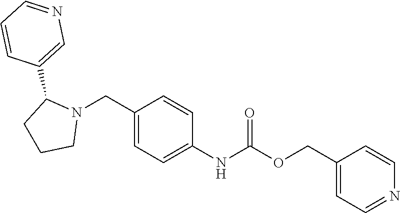 [SMILES: O=C(Nc1ccc(CN2CCC[C@@H]2c2cccnc2)cc1)OCc1ccncc1] |
| | | | Compound 3: pyridin-4-ylmethylN-(4-{[(2R)-2- |
| | | | (pyridin-3-yl)pyrrolidin-1- |
| | | | yl]methyl}phenyl)carbamate.LCMS-ESI (POS.) |
| | | | m/z: 389.0 [M + H]⁺. ¹H NMR (300 MHz, DMSO-d6) δ |
| | | | 9.85 (s, 1H), 8.63-8.54 (m, 3H), 8.46 (dd, J = 4.7, 1.7 |
| | | | Hz, 1H), 7.84 (dt, J = 7.8, 2.0 Hz, 1H), 7.43-7.33 (m, |
| | | | 5H), 7.15 (d, J = 8.4 Hz, 2H), 5.19 (s, 2H), 3.59 (d, J = |
| | | | 13.1 Hz, 1H), 3.43 (t, J = 8.1 Hz, 1H), 3.05 (d, J = 13.1 |
| | | | Hz, 1H), 2.96 (t, J = 6.9 Hz, 1H), 2.20 (q, J = 8.9 Hz, |
| | | | 2H), 1.78 (s, 2H), 1.69-1.53 (m, 1H). |
| 2 | (R)-3- (pyrrolidin- 2-yl) pyridine | 4- (hydroxymethyl) benzamide | 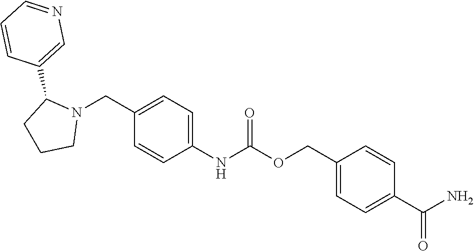 [SMILES: NC(=O)c1ccc(COC(=O)Nc2ccc(CN3CCC[C@@H]3c3cccnc3)cc2)cc1] |
| | | | Compound 2: (4-carbamoylphenyl)methyl N-(4- |
| | | | {[(2R)-2-(pyridin-3-yl)pyrrolidin-1- |
| | | | yl]methyl}phenyl)carbamate. LCMS-ESI (POS.) |
| | | | m/z: 431.0 [M + H]⁺. ¹H NMR (300 MHz, DMSO-d6) δ |
| | | | 9.75 (s, 1H), 8.63-8.56 (m, 1H), 8.46 (dd, J = 4.7, 1.7 |
| | | | Hz, 1H), 8.01-7.94 (m, 1H), 7.93-7.79 (m, 3H), 7.52- |
| | | | 7.43 (m, 2H), 7.43-7.32 (m, 4H), 7.14 (d, J = 8.4 |
| | | | Hz, 2H), 5.19 (s, 2H), 3.58 (d, J = 13.1 Hz, 1H), 3.43 |
| | | | (t, J = 8.1 Hz, 1H), 3.05 (d, J = 13.1 Hz, 1H), 3.01- |
| | | | 2.89 (m, 1H), 2.20 (q, J = 8.8 Hz,2H), 1.78 (s, 2H), |
| | | | 1.69-1.50 (m, 1H). |
| 5 | (R)-3- (pyrrolidin- 2-yl) pyridine | 1,4- phenylene- dimethanol | 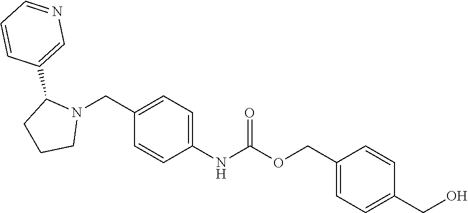 [SMILES: O=C(Nc1ccc(CN2CCC[C@@H]2c2cccnc2)cc1)OCc1ccc(CO)cc1] |
| | | | Compound 5: [4-(hydroxymethyl)phenyl]methyl N- |
| | | | (4-{[(2R)-2-(pyridin-3-yl)pyrrolidin-1- |
| | | | yl]methyl}phenyl)carbamate. LCMS-ESI (POS.) |
| | | | m/z: 418.0 [M + H]⁺. ¹H NMR (300 MHz, DMSO-d₆) δ |
| | | | 9.68 (s, 1H), 8.59 (d, J = 2.2 Hz, 1H), 8.46 (dd, J = 4.7, |
| | | | 1.7 Hz, 1H), 7.84 (dt, J = 7.9, 2.0 Hz, 1H), 7.43-7.28 |
| | | | (m, 7H), 7.13 (d, J = 8.5 Hz, 2H), 5.19 (t, J = 5.7 Hz, |
| | | | 1H), 5.11 (s, 2H), 4.49 (d, J = 5.7 Hz, 2H), 3.58 (d, J = |
| | | | 13.1 Hz, 1H), 3.43 (t, J = 8.1 Hz, 1H), 3.04 (d, J = 13.1 |
| | | | Hz, 1H), 2.95 (t, J = 7.2 Hz, 1H), 2.20 (q, J = 8.8 Hz, |
| | | | 2H), 1.78 (m, 2H), 1.59 (m, 1H). |
| 168 | 1-(4- amino- benzyl)-4- methyl- piperazin- 2-one | (4- methoxyphenyl) methanol | 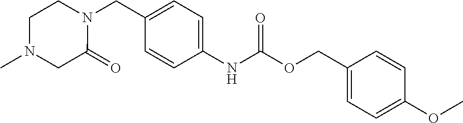 |
| | | | Compound 168: (4-methoxyphenyl)methyl N-{4- |
| | | | [(4-methyl-2-oxopiperazin-1- |
| | | | yl)methyl]phenyl}carbamate. LCMS-ESI (POS.) |
| | | | m/z: 384.0 [M + H]⁺. ¹H NMR (300 MHz, DMSO-d6) |
| | | | δ 9.69 (s, 1H), 7.39 (dd, J = 15.0, 8.1 Hz, 4H), 7.13 (d, |
| | | | J = 8.1 Hz, 2H), 6.95 (d, J = 8.2 Hz, 2H), 5.07 (s, 2H), |
| | | | 4.43 (s, 2H), 3.76 (s, 3H), 3.15 (t, J = 5.6 Hz, 2H), 2.99 |
| | | | (s, 2H), 2.55 (d, J = 5.5 Hz, 2H), 2.20 (s, 3H). |
| 222 | 3- (methyl- sulfonyl) azetidine | (4- chlorophenyl) methanol | 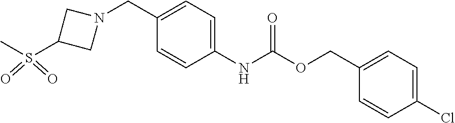 [SMILES: CS(=O)(=O)C1CN(Cc2ccc(NC(=O)OCc3ccc(Cl)cc3)cc2)C1] |
| | | | Compound 222: (4-chlorophenyl)methyl N-{4-[(3- |
| | | | methanesulfonylazetidin-1- |
| | | | yl)methyl]phenyl}carbamate. LCMS-ESI (POS.) |
| | | | m/z: 410.0 [M + H]⁺. ¹H NMR (300 MHz, DMSO-d6) δ |
| | | | 9.76 (s, 1H), 7.42 (d, J = 21.8 Hz, 6H), 7.17 (d, J = 8.5 |
| | | | Hz, 2H), 5.13 (s, 2H), 4.13 (p, J = 7.2 Hz, 1H), 3.44 (d, |
| | | | J = 8.0 Hz, 2H), 3.38-3.28 (m, 4H), 2.94 (s, 3H). |
| 324 | 3- (methyl- sulfonyl) azetidine | (4- methoxyphenyl) methanol | 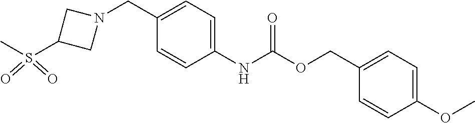 |
| | | | Compound 324: (4-methoxyphenyl)methyl N-{4- |
| | | | [(3-methanesulfonylazetidin-1- |
| | | | yl)methyl]phenyl}carbamate. LCMS-ESI (POS.) |
| | | | m/z: 405.1 [M + H]⁺. ¹H NMR (300 MHz, DMSO-d6) δ |
| | | | 9.67 (s, 1H), 7.36 (m, 4H) 7.15 (d, J = 8.5 Hz, 2H), 6.93 |
| | | | (d, J = 8.5 Hz, 2H), 5.06 (s, 2H), 4.13 (p, J = 7.2 Hz, |
| | | | 1H), 3.75 (s, 3H), 3.54-3.40 (m, 4H), 3.35 (m, 2H), |
| | | | 2.94 (s, 3H). |
| 228 | (S)-3- amino-1- methyl- pyrrolidin- 2-one | (4- chlorophenyl) methanol | 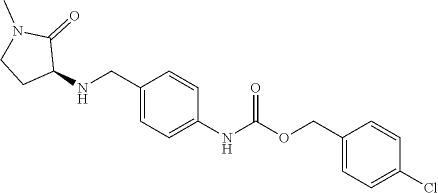 [SMILES: CN1CC[C@H](NCc2ccc(NC(=O)OCc3ccc(Cl)cc3)cc2)C1=O] |
| | | | Compound 228: 4-chlorobenzyl (S)-(4-(((1-methyl- |
| | | | 2-oxopyrrolidin-3- |
| | | | yl)amino)methyl)phenyl)carbamate. LRMS (ES) |
| | | | m/z: 388 [M + H]⁺. ¹HNMR (300 MHz, DMSO-d₆) δ |
| | | | 9.72 (s, 1H), 7.42 (m, 6H), 7.23 (d, J = 8.4 Hz, 2H), |
| | | | 5.13 (s, 2H), 3.71 (d, J = 6.0 Hz, 2H), 3.29-3.11 (m, |
| | | | 3H), 2.71 (s, 3H), 2.27 (s, 1H), 2.20-2.05 (m, 1H), |
| | | | 1.63 (dq, J = 12.1, 8.7 Hz, 1H). |
| 284 | 1- methyl- piperazin- 2-one | (4- chlorophenyl) methanol | 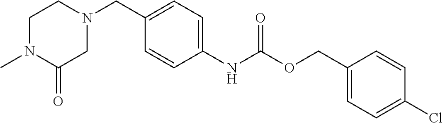 [SMILES: CN1CCN(Cc2ccc(NC(=O)OCc3ccc(Cl)cc3)cc2)CC1=O] |
| | | | Compound 284: (4-chlorophenyl)methyl N-{4-[(4- |
| | | | methyl-3-oxopiperazin-1- |
| | | | yl)methyl]phenyl}carbamate. LCMS-ESI (POS.) |
| | | | m/z: 388.0 [M + H]⁺. ¹H NMR (400 MHz, DMSO-d6) δ |
| | | | 9.80 (s, 1H), 7.45 (d, J = 10.6 Hz, 5H), 7.30 (s, 1H), |
| | | | 7.21 (d, J = 8.1 Hz, 2H), 5.14 (s, 2H), 3.44 (s, 2H), 3.23 |
| | | | (t, J = 5.4 Hz, 2H), 2.91 (s, 2H), 2.80 (s, 3H), 2.59 (t, |
| | | | J = 5.5 Hz, 2H). |
| 325 | 1- methyl- piperazin- 2-one | (4- methoxyphenyl) methanol | 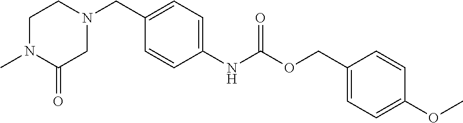 |
| | | | Compound 325: (4-methoxyphenyl)methyl N-{4- |
| | | | [(4-methyl-3-oxopiperazin-1- |
| | | | yl)methyl]phenyl}carbamate. LCMS-ESI (POS.) |
| | | | m/z: 384.0 [M + H]⁺. ¹H NMR (300 MHz, DMSO-d6) δ |
| | | | 9.69 (s, 1H), 7.39 (dd, J = 15.4, 8.4 Hz, 4H), 7.20 (d, |
| | | | J = 8.3 Hz, 2H), 6.99-6.90 (m, 2H), 5.06 (s, 2H), 3.75 |
| | | | (s, 3H), 3.44 (s, 2H), 3.33-3.18 (m, 2H), 2.90 (s, 2H), |
| | | | 2.80 (s, 3H), 2.59 (t, J = 5.6 Hz, 2H). |
| | tert- butyl piperazine- 1- carboxylate | (4- chlorophenyl) methanol | 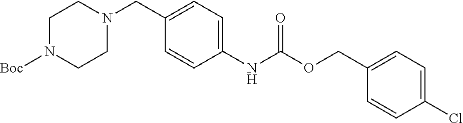 [SMILES: CC(C)(C)OC(=O)N1CCN(Cc2ccc(NC(=O)OCc3ccc(Cl)cc3)cc2)CC1] |
| | | | Intermediate 25.1: tert-butyl 4-(4-((((4- |
| | | | chlorobenzyl)oxy)carbonyl)amino)benzyl)piperazine- |
| | | | 1-carboxylate. LRMS (ES) m/z: 404 [M + H-tBu]⁺. |
| | tert- butyl piperazine- 1- carboxylate | (4- methoxyphenyl) methanol | 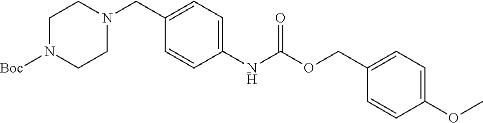 [SMILES: COc1ccc(COC(=O)Nc2ccc(CN3CCN(C(=O)OC(C)(C)C)CC3)cc2)cc1] |
| | | | Intermediate 26.1: tert-butyl 4-(4-((((4- |
| | | | methoxybenzyl)oxy)carbonyl)amino)benzyl) |
| | | | piperazine-1-carboxylate. LRMS (ES) m/z: 400 [M + H- |
| | | | tBu]⁺. |
| 341 | (S)- pyrrolidin- 2- ylmethanol | (4- methoxyphenyl) methanol | 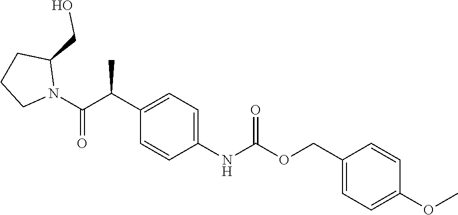 [SMILES: COc1ccc(COC(=O)Nc2ccc([C@H](C)C(=O)N3CCC[C@H]3CO)cc2)cc1] |
| | | | Compound 341: (4-methoxyphenyl)methyl N-{4- |
| | | | [(2S)-1-[(2S)-2-(hydroxymethyl)pyrrolidin-1-yl]-1- |
| | | | oxopropan-2-yl]phenyl}carbamate LCMS-ESI |
| | | | (POS.) m/z: 413.0 [M + H]⁺. ¹H NMR (400 MHz, |
| | | | DMSO-d₆) δ 9.64 (s, 1H), 7.36 (dd, J = 8.7, 3.3 Hz, |
| | | | 4H), 7.21-7.09 (m, 2H), 6.99-6.90 (m, 2H), 5.06 (s, |
| | | | 2H), 4.70 (dd, J = 6.3, 4.7 Hz, 1H), 3.98 (d, J = 6.8 Hz, |
| | | | 1H), 3.90-3.78 (m, 1H), 3.75 (s, 3H), 3.53-3.34 (m, |
| | | | 2H), 3.05 (dd, J = 9.9, 6.0 Hz, 2H), 1.76 (d, J = 3.9 Hz, |
| | | | 3H), 1.24 (dd, J = 15.6, 6.9 Hz, 3H). |
| 47 | (S)- pyrrolidin- 2- ylmethanol | (4- methoxyphenyl) methanol | 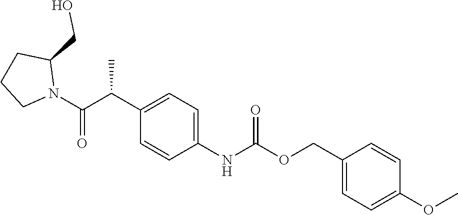 [SMILES: COc1ccc(COC(=O)Nc2ccc([C@@H](C)C(=O)N3CCC[C@H]3CO)cc2)cc1] |
| | | | Compound 47: {[4-(1,1-dioxothietan-3- |
| | | | yl)phenyl]amino}-N-[(4- |
| | | | methoxyphenyl)methyl]carboxamide. LCMS-ESI |
| | | | (POS.) m/z: 413.0 [M + H]⁺. ¹HNMR (400 MHz, |
| | | | DMSO-d₆) δ 9.64 (s, 1H), 7.41-7.31 (m, 4H), 7.15 (d, |
| | | | J = 8.6 Hz, 2H), 6.99-6.90 (m, 2H), 5.05 (s, 2H), 4.77 |
| | | | (dt, J = 20.2, 5.7 Hz, 1H), 3.89 (s, 1H), 3.80 (t, J = 6.8 |
| | | | Hz, 1H), 3.75 (s, 3H), 3.51 (dd, J = 9.9, 5.4 Hz, |
| | | | 2H),3.29(d, J = 8.2 Hz, 1H), 3.06 (q, J = 8.2 Hz, 1H), |
| | | | 1.88-1.76 (m, 2H), 1.63 (d, J = 8.2 Hz, 3H), 1.25 (d, |
| | | | J = 6.8 Hz, 3H). |

### Suzuki Coupling

### Example J

### Preparation of 4-chlorobenzyl (4-(thiazol-2-ylmethyl)phenyl)carbamate (Compound 336)

### Step 1: Synthesis of 4-chlorobenzyl (4-(4,4,5,5-tetramethyl-1,3,2-dioxaborolan-2-yl)phenyl)carbamate

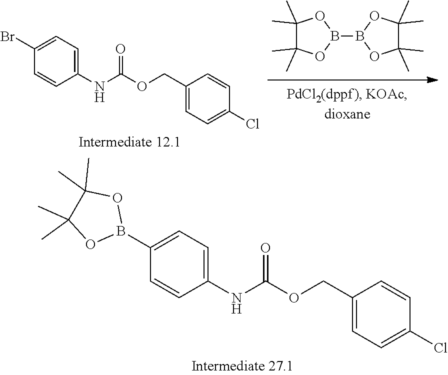

To a mixture of 4-chlorobenzyl (4-bromophenyl)carbamate (2.04 g, 6.00 mmol, 1.00 equiv), bis(pinacolato)diboron (2.29 g, 9.00 mmol, 1.50 equiv) in dioxane (20 mL) was added KOAc (0.88 g, 9.00 mmol, 1.50 equiv), PdCl₂(dppf) (0.44 g, 0.60 mmol, 0.10 equiv). The resulting mixture was degassed for 10 min. Then it was heated at MW at 130° C. for 60 min. The reaction mixture was extracted with EtOAc (100 mL) and brine (50 mL). The organic layer was concentrated and purified on silica gel with 25% EtOAc/Hex to afford the desired product 4-chlorobenzyl (4-(4,4,5,5-tetramethyl-1,3,2-dioxaborolan-2-yl)phenyl)carbamate as a yellowish solid (1.25 g, 54%). LRMS (ES) m/z: 388 [M+H]⁺.

### Step 2: Synthesis of 4-chlorobenzyl (4-(thiazol-2-ylmethyl)phenyl)carbamate

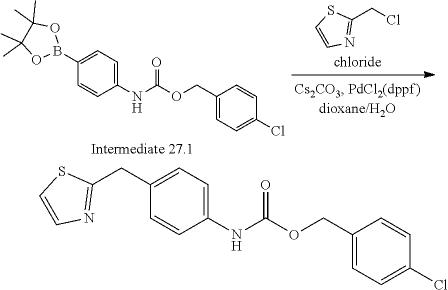 [SMILES: CC1(C)OB(c2ccc(NC(=O)OCc3ccc(Cl)cc3)cc2)OC1(C)C ; ClCc1nccs1 ; O=C(Nc1ccc(Cc2nccs2)cc1)OCc1ccc(Cl)cc1]

To a mixture of 4-chlorobenzyl (4-(4,4,5,5-tetramethyl-1,3,2-dioxaborolan-2-yl)phenyl)carbamate (78 mg, 0.20 mmol, 1.00 equiv), 2-(chloromethyl)thiazole (40 mg, 0.30 mmol, 1.50 equiv), Cs₂CO₃ (77 mg, 0.24 mmol, 1.20 equiv) in dioxane (2 mL) and H₂O (1 mL) was added PdCl₂(dppf) (15 mg, 0.02 mmol, 0.10 equiv). The resulting mixture was degassed for 2 min. Then it was heated at 130° C. at MW for 30 min. The mixture was extracted with EtOAc (0 mL) and brine (3 mL). The organic layer was concentrated and purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford 4-chlorobenzyl (4-(thiazol-2-ylmethyl)phenyl)carbamate (21 mg, 29%) as a white solid. LRMS (ES) m/z: 359 [M+H]3. ¹H NMR (400 MHz, Methanol-d₄) δ 7.70 (d, J=3.4 Hz, 1H), 7.52-7.33 (m, 6H), 7.33-7.16 (m, 2H), 5.17 (s, 2H), 4.31 (s, 2H).

Compounds in the following table were prepared in a similar manner as Compound 336, using the intermediates and chlorides as listed.

| Co# | Intermediate | Chloride | Structure, Name and Data |
|---|---|---|---|
| 314 | 27.1 | 5-(chloromethyl)- 1-methyl- 1H-pyrazole | 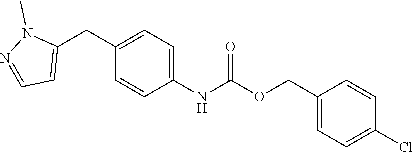 [SMILES: Cn1nccc1Cc1ccc(NC(=O)OCc2ccc(Cl)cc2)cc1] |
| | | | Compound 314: 4-chlorobenzyl (4- |
| | | | ((1-methyl-1H-pyrazol-5- |
| | | | yl)methyl)phenyl)carbamate LRMS |
| | | | (ES) m/z: 356[M + H]+. ¹H NMR (400 |
| | | | MHz, Methanol-d₄) δ 7.48-7.34 (m, |
| | | | 7H), 7.21-6.93 (m, 2H), 6.05 (d, J = |
| | | | 1.9 Hz, 1H), 5.17 (s, 2H), 4.01 (s, 2H), |
| | | | 3.71 (s, 3H). |
| 351 | 27.1 | 5-(chloromethyl)thiazole | 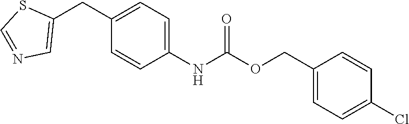 [SMILES: O=C(Nc1ccc(Cc2cncs2)cc1)OCc1ccc(Cl)cc1] |
| | | | Compound 351: 4-chlorobenzyl (4- |
| | | | (thiazol-5- |
| | | | ylmethyl)phenyl)carbamate LRMS |
| | | | (ES) m/z: 359 [M + H]⁺. ¹H NMR (400 |
| | | | MHz, Methanol-d₄) δ 8.85 (s, 1H), 7.75- |
| | | | 7.65 (m, 1H), 7.48-7.32 (m, 6H), |
| | | | 7.30-7.07 (m, 2H), 5.17 (s, 2H), 4.18 |
| | | | (s, 2H). |
| 303 | 27.1 | 2-(chloromethyl)pyrazine | 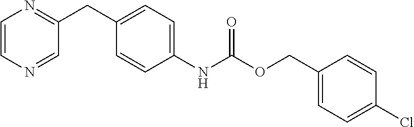 [SMILES: O=C(Nc1ccc(Cc2cnccn2)cc1)OCc1ccc(Cl)cc1] |
| | | | Compound 303: 4-chlorobenzyl (4- |
| | | | (pyrazin-2- |
| | | | ylmethyl)phenyl)carbamate LRMS |
| | | | (ES) m/z: 354 [M + H]⁺. ¹H NMR (400 |
| | | | MHz, Methanol-d₄) δ 8.53 (d, J = 1.6 |
| | | | Hz, 2H), 8.45 (d, J = 2.2 Hz, 1H), 7.50- |
| | | | 7.33 (m, 6H), 7.33-6.99 (m, 2H), |
| | | | 5.16 (s, 2H), 4.14 (s, 2H). |
| 350 | 27.1 | 5-(chloromethyl) isothiazole | 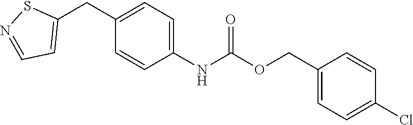 [SMILES: O=C(Nc1ccc(Cc2ccns2)cc1)OCc1ccc(Cl)cc1] |
| | | | Compound 350: 4-chlorobenzyl (4- |
| | | | (isothiazol-5- |
| | | | ylmethyl)phenyl)carbamate LRMS |
| | | | (ES) m/z: 359 [M + H]⁺. ¹H NMR (400 |
| | | | MHz, Methanol-d₄) δ 8.37 (d, J = 1.6 |
| | | | Hz, 1H), 7.41 (m, 6H), 7.24 (d, J = 8.3 |
| | | | Hz, 2H), 7.13 (d, J = 1.6 Hz, 1H), 5.18 |
| | | | (s, 2H), 4.28 (s, 2H). |
| 353 | 27.1 | 4-(chloromethyl)oxazole | 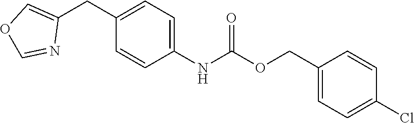 [SMILES: O=C(Nc1ccc(Cc2cocn2)cc1)OCc1ccc(Cl)cc1] |
| | | | Compound 353: 4-chlorobenzyl (4- |
| | | | (oxazol-4-ylmethyl)phenyl) LRMS |
| | | | (ES) m/z: 343 [M + H]⁺. ¹H NMR (400 |
| | | | MHz, Methanol-d₄) δ 8.14 (s, 1H), 7.62 |
| | | | (d, J = 1.2 Hz, 1H), 7.38 (dd, J = 9.6, |
| | | | 5.2 Hz, 6H), 7.19 (d, J = 8.3 Hz, 2H), |
| | | | 5.16 (s, 2H), 3.84 (s, 2H). |
| 279 | 27.1 | 2-(chloromethyl) pyrimidine | 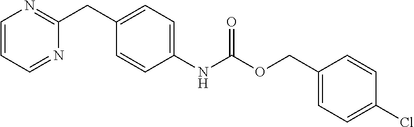 [SMILES: O=C(Nc1ccc(Cc2ncccn2)cc1)OCc1ccc(Cl)cc1] |
| | | | Compound 279: 4-chlorobenzyl (4- |
| | | | (pyrimidin-2- |
| | | | ylmethyl)phenyl)carbamate. LRMS |
| | | | (ES) m/z: 354.1 [M + H]⁺. ¹H NMR (400 |
| | | | MHz, Methanol-d₄) δ 8.73 (d, J = 5.0 Hz, |
| | | | 2H), 7.45-7.31 (m, 7H), 7.24 (d, J = 8.3 |
| | | | Hz, 2H), 5.15 (s, 2H), 4.21 (s, 2H). |

### Amine Deprotection

### Example K

### Preparation of 4-chlorobenzyl (4-(piperidin-4-ylmethyl)phenyl)carbamate hydrochloride (Compound 231/Intermediate 6.2)

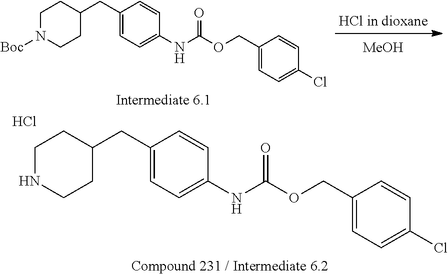 [SMILES: "MeOH" ; CC(C)(C)OC(=O)N1CCC(Cc2ccc(NC(=O)OCc3ccc(Cl)cc3)cc2)CC1 ; Cl ; O=C(Nc1ccc(CC2CCNCC2)cc1)OCc1ccc(Cl)cc1]

To a solution of tert-butyl 4-(4-((((4-chlorobenzyl)oxy)carbonyl)amino)benzyl)piperidine-1-carboxylate (1.30 g, 2.84 mmol, 1.00 equiv) in MeOH (2 mL) at 0° C. was added 4M HCl/Dioxane (8.52 mL, 34.08 mmol, 12 equiv) slowly. The resulting mixture was warmed up to rt and stirred for 1 h. The solvents were removed and the residue 4-chlorobenzyl (4-(piperidin-4-ylmethyl)phenyl)carbamate hydrochloride was used directly in the next steps without further purification. LRMS (ES) m/z: 359 [M+H]⁺.

Compounds in the following table were prepared in a similar manner as Compound 231/intermediate 6.2, using the intermediates as listed.

| Co | Inter- | |
|---|---|---|
| # | mediate | Structure, Name and Data |
| | 7.1 | 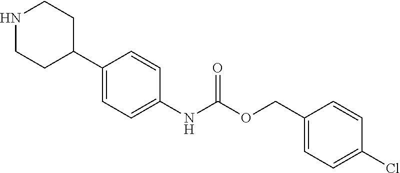 [SMILES: O=C(Nc1ccc(C2CCNCC2)cc1)OCc1ccc(Cl)cc1] |
| | | Intermediate 7.2: 4-chlorobenzyl (4-(piperidin-4- |
| | | yl)phenyl)carbamate. LCMS-APCI (POS.) m/z: 345.1 [M + H]+. |
| 320 | 8.1 | 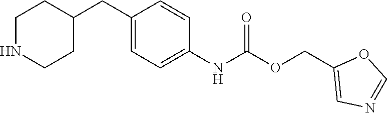 [SMILES: O=C(Nc1ccc(CC2CCNCC2)cc1)OCc1cnco1] |
| | | Compound 320/Intermediate 8.2: oxazol-5-ylmethyl (4-(piperidin- |
| | | 4-ylmethyl)phenyl)carbamate. LRMS (ES) m/z: 316.1 [M + H]⁺. ¹H |
| | | NMR (400 MHz, Methanol-d₄) δ 8.46 (s, 1H), 8.24 (s, 1H), 7.38 (d, J = |
| | | 8.0 Hz, 2H), 7.25 (s, 1H), 7.13 (d, J = 8.1 Hz, 2H), 5.25 (s, 2H), 3.37 (d, |
| | | J = 12.5 Hz, 2H), 2.93 (td, J = 13.0, 2.7 Hz, 2H), 2.58 (d, J = 6.6 Hz, 2H), |
| | | 2.00-1.79 (m, 2H), 1.43 (qd, J = 11.9, 6.0 Hz, 2H). |
| | 9.1 | 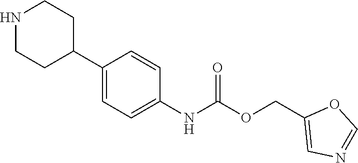 [SMILES: O=C(Nc1ccc(C2CCNCC2)cc1)OCc1cnco1] |
| | | Intermediate 9.2: oxazol-5-ylmethyl (4-(piperidin-4- |
| | | yl)phenyl)carbamate. LRMS (ES) m/z: 302.1 [M + H]⁺. |
| | 10.1 | 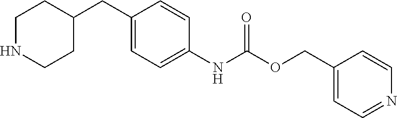 [SMILES: O=C(Nc1ccc(CC2CCNCC2)cc1)OCc1ccncc1] |
| | | Intermediate 10.2: pyridin-4-ylmethyl (4-(piperidin-4- |
| | | ylmethyl)phenyl)carbamate. LCMS-APCI (POS.) m/z: 326.1 |
| | | [M + H]+. |
| 252 | 11.1 | 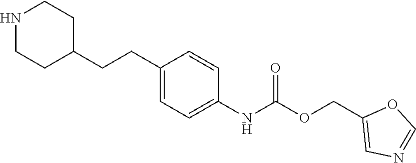 [SMILES: O=C(Nc1ccc(CCC2CCNCC2)cc1)OCc1cnco1] |
| | | Compound 252/Intermediate 11.2: oxazol-5-ylmethyl (4-(2- |
| | | (piperidin-4-yl)ethyl)phenyl)carbamate hydrochloride. LRMS (ES) |
| | | m/z: 330.1 [M + H]⁺. ¹H NMR (400 MHz, DMSO-d₆) δ 9.73 (s, 1H), |
| | | 8.89 (s, 1H), 8.66 (s, 1H), 8.43 (s, 1H), 7.35 (d, J = 8.1 Hz, 2H), 7.30 (s, |
| | | 1H), 7.18-7.02 (m, 2H), 5.21 (s, 2H), 3.21 (d, J = 12.6 Hz, 2H), 2.79 |
| | | (q, J = 12.0 Hz, 2H), 2.53 (m, 2H), 1.82 (d, J = 13.6 Hz, 2H), 1.49 (t, |
| | | J = 6.5 Hz, 3H), 1.41-1.24 (m, 2H). |
| | 13.1 | 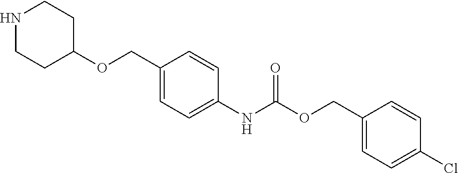 [SMILES: O=C(Nc1ccc(COC2CCNCC2)cc1)OCc1ccc(Cl)cc1] |
| | | Intermediate 13.2: 4-chlorobenzyl (4-((piperidin-4- |
| | | yloxy)methyl)phenyl)carbamate. |
| | | LCMS-APCI (POS.) m/z: 375. [M + H]⁺. |
| | 14.1 | 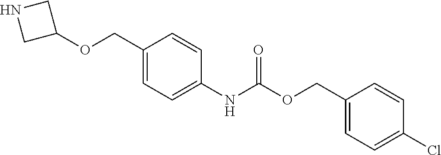 [SMILES: O=C(Nc1ccc(COC2CNC2)cc1)OCc1ccc(Cl)cc1] |
| | | Intermediate 14.2: 4-chlorobenzyl (4-((azetidin-3- |
| | | yloxy)methyl)phenyl)carbamate. LCMS-APCI (POS.) m/z: 347.1 |
| | | [M + H]⁺. |
| | 15.1 | 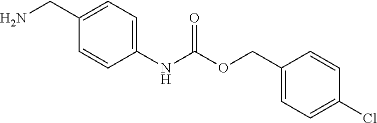 [SMILES: NCc1ccc(NC(=O)OCc2ccc(Cl)cc2)cc1] |
| | | Intermediate 15.2: (4-chlorophenyl)methyl N-[4- |
| | | (aminomethyl)phenyl]carbamate. LCMS-ESI (NEG.) m/z: 289.0 |
| | | [M − H]⁻. |
| | 16.1 | 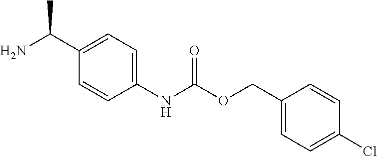 [SMILES: C[C@H](N)c1ccc(NC(=O)OCc2ccc(Cl)cc2)cc1] |
| | | Intermediate 16.2: (1S)-1-[4-({[(4- |
| | | chlorophenyl)methoxy]carbonyl}amino)phenyl]ethanaminium |
| | | chloride. LCMS-APCI (POS.) m/z: 288.0 [M + H—NH₃]⁺. |
| | 17.1 | 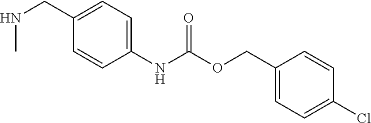 [SMILES: CNCc1ccc(NC(=O)OCc2ccc(Cl)cc2)cc1] |
| | | Intermediate 17.2: (4-chlorophenyl)methyl N-{4- |
| | | [(methylamino)methyl]phenyl}carbamate. LCMS-ES (POS.) m/z: |
| | | 305.0 [M + H]⁺. |
| | 18.1 | 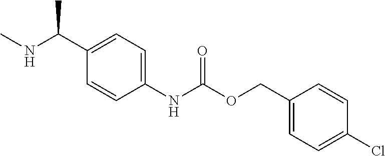 [SMILES: CN[C@@H](C)c1ccc(NC(=O)OCc2ccc(Cl)cc2)cc1] |
| | | Intermediate 18.2: 4-chlorobenzyl (S)-(4-(1- |
| | | (methylamino)ethyl)phenyl)carbamate. LCMS-ES (POS.) m/z: 319.0 |
| | | [M + H]⁺. |
| | 19.1 | 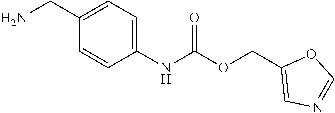 [SMILES: NCc1ccc(NC(=O)OCc2cnco2)cc1] |
| | | Intermediate 19.2: 1,3-oxazol-5-ylmethyl N-[4- |
| | | (aminomethyl)phenyl]carbamate. LCMS-ESI (POS.) m/z: 231.0 |
| | | [M + H—NH₃]⁺. |
| | 20.1 | 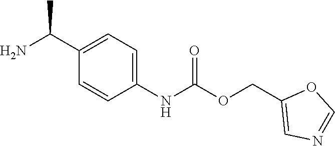 [SMILES: C[C@H](N)c1ccc(NC(=O)OCc2cnco2)cc1] |
| | | Intermediate 20.2: 1,3-oxazol-5-ylmethyl N-{4-[(1S)-1- |
| | | aminoethyl]phenyl}carbamate. LCMS-APCI (POS.) m/z: 245.1 |
| | | [M + H—NH₃]⁺. ¹H NMR (400 MHz, DMSO-d₆) δ 9.93 (s, 1H), 8.43 (s, |
| | | 1H), 8.35 (s, 3H), 7.48 (d, J = 8.2 Hz, 2H), 7.41 (d, J = 8.1 Hz, 2H), 7.31 |
| | | (s, 1H), 5.22 (s, 2H), 4.32 (p, J = 6.5 Hz, 1H), 1.48 (d, J = 6.7 Hz, 3H). |
| | 25.1 | 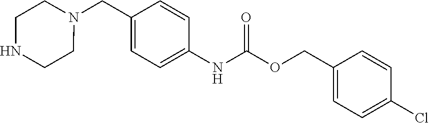 [SMILES: O=C(Nc1ccc(CN2CCNCC2)cc1)OCc1ccc(Cl)cc1] |
| | | Intermediate 25.2: 4-chlorobenzyl (4-(piperazin-1- |
| | | ylmethyl)phenyl)carbamate. LRMS (ES) m/z: 360.1[M + H] |
| | 26.1 | 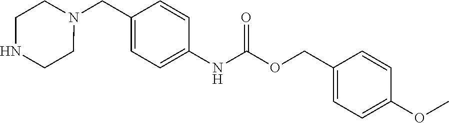 [SMILES: COc1ccc(COC(=O)Nc2ccc(CN3CCNCC3)cc2)cc1] |
| | | Intermediate 26.2: 4-methoxybenzyl (4-(piperazin-1- |
| | | ylmethyl)phenyl)carbamate. LRMS (ES) m/z: 356.1[M + H]⁺. |
| | 29.1 | 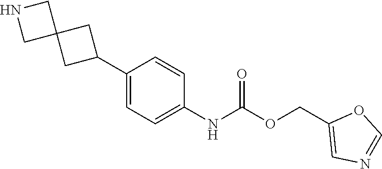 [SMILES: O=C(Nc1ccc(C2CC3(CNC3)C2)cc1)OCc1cnco1] |
| | | Intermediate 31.1: oxazol-5-ylmethyl (4-(2-azaspiro[3.3]heptan-6- |
| | | yl)phenyl)carbamate |
| | | LRMS (ES) m/z: 314.1 [M + H]⁺ |
| | 29.2 | 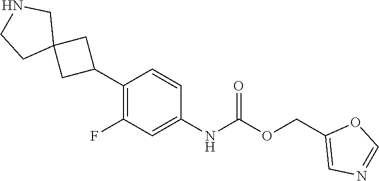 [SMILES: O=C(Nc1ccc(C2CC3(CCNC3)C2)c(F)c1)OCc1cnco1] |
| | | Intermediate 30.1: oxazol-5-ylmethyl (3-fluoro-4-(6- |
| | | azaspiro[3.4]octan-2-yl)phenyl)carbamate |
| | | LRMS (ES) m/z: 346.1[M + H]⁺. |
| | 29.3 | 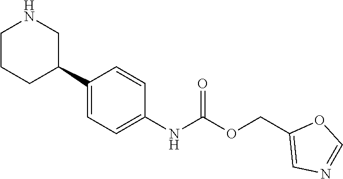 [SMILES: O=C(Nc1ccc([C@H]2CCCNC2)cc1)OCc1cnco1] |
| | | Intermediate 30.2: oxazol-5-ylmethyl (R)-(4-(piperidin-3- |
| | | yl)phenyl)carbamate |
| | | LRMS (ES) m/z: 301.1[M + H]⁺. |
| 378 | 29.4 | 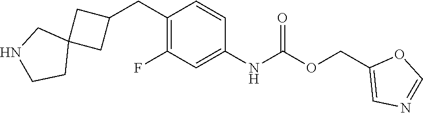 [SMILES: O=C(Nc1ccc(CC2CC3(CCNC3)C2)c(F)c1)OCc1cnco1] |
| | | Compound 378: oxazol-5-ylmethyl (4-((6-azaspiro[3.4]octan-2- |
| | | yl)methyl)-3-fluorophenyl)carbamate |
| | | LRMS (ES) m/z: 360.1[M + H]⁺. |
| | | ¹H NMR (400 MHz, DMSO-d₆) δ 9.98 (s, 1H), 8.45 (s, 1H), 8.36 (s, |
| | | 1H), 7.35-7.29 (m, 2H), 7.21-7.13 (m, 2H), 5.25 (s, 2H), 3.20 (s, |
| | | 2H), 3.09-3.05 (m, 2H), 2.97 (s, 1H), 2.03-1.87 (m, 4H), 1.85-1.69 |
| | | (m, 4H). |
| | 29.5 | 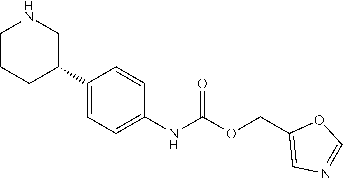 [SMILES: O=C(Nc1ccc([C@@H]2CCCNC2)cc1)OCc1cnco1] |
| | | Intermediate 30.3: oxazol-5-ylmethyl (S)-(4-(piperidin-3- |
| | | yl)phenyl)carbamate |
| | | LRMS (ES) m/z: 302.1[M + H]⁺. |
| | 29.6 | 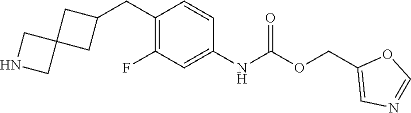 [SMILES: O=C(Nc1ccc(CC2CC3(CNC3)C2)c(F)c1)OCc1cnco1] |
| | | Intermediate 30.4: oxazol-5-ylmethyl (4-((2-azaspiro[3.3]heptan-6- |
| | | yl)methyl)-3-fluorophenyl)carbamate |
| | | LRMS (ES) m/z: 346.1[M + H]⁺. |
| | 29.7 | 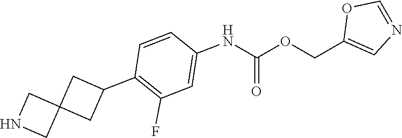 [SMILES: O=C(Nc1ccc(C2CC3(CNC3)C2)c(F)c1)OCc1cnco1] |
| | | Intermediate 30.4-a: oxazol-5-ylmethyl (3-fluoro-4-(2- |
| | | azaspiro[3.3]heptan-6-yl)phenyl)carbamate |
| | | LRMS (ES) m/z: 332.1[M + H]⁺. |
| | 29.8 | 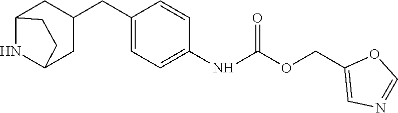 [SMILES: O=C(Nc1ccc(CC2CC3CCC(C2)N3)cc1)OCc1cnco1] |
| | | Intermediate 30.5: oxazol-5-ylmethyl (3-fluoro-4-(2- |
| | | azaspiro[3.3]heptan-6-yl)phenyl)carbamate |
| | | LRMS (ES) m/z: 342.1[M + H]⁺. |
| | 29.9 | 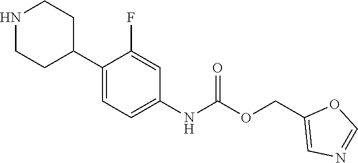 [SMILES: O=C(Nc1ccc(C2CCNCC2)c(F)c1)OCc1cnco1] |
| | | Intermediate 30.6: oxazol-5-ylmethyl (3-fluoro-4-(piperidin-4- |
| | | yl)phenyl)carbamate |
| | | LRMS (ES) m/z: 320.1[M + H]⁺. |
| | 29.10 | 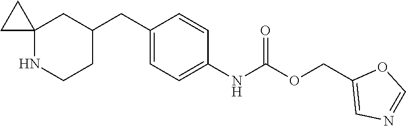 [SMILES: O=C(Nc1ccc(CC2CCNC3(CC3)C2)cc1)OCc1cnco1] |
| | | Intermediate 30.7: oxazol-5-ylmethyl (4-((4-azaspiro[2.5]octan-7- |
| | | yl)methyl)phenyl)carbamate hydrochloride |
| | | LRMS (ES) m/z: 342.1[M + H]⁺. |
| | | ¹H NMR (400 MHz, DMSO-d₆) δ 8.22 (s, 1H), 7.35 (d, J = 8.2 Hz, 2H), |
| | | 7.23 (s, 1H), 7.11 (d, J = 8.3 Hz, 2H), 5.23 (s, 2H), 4.58 (s, 2H), 3.04 |
| | | (td, J = 12.7, 2.90 Hz, 1H), 2.59 (d, J = 7.2 Hz, 2H), 2.12-1.77 (m, |
| | | 3H), 1.41 (qd, J = 12.5, 4.0 Hz, 1H), 1.32-1.22 (m, 1H), 1.00-0.87 |
| | | (m, 2H), 0.85-0.77 (m, 1H), 0.75-0.65 (m, 1H). |
| 426 | 29.11 | 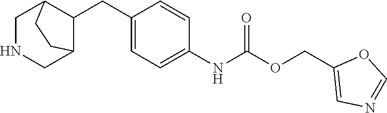 [SMILES: O=C(Nc1ccc(CC2C3CCC2CNC3)cc1)OCc1cnco1] |
| | | Compound 426: oxazol-5-ylmethyl (4-((3-azabicyclo[3.2.1]octan-8- |
| | | yl)methyl)phenyl)carbamate |
| | | LRMS (ES) m/z: 342.1[M + H]⁺. |
| | 29.12 | 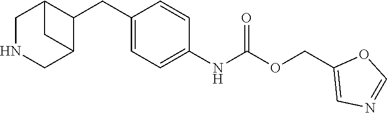 [SMILES: O=C(Nc1ccc(CC2C3CNCC2C3)cc1)OCc1cnco1] |
| | | Intermediate 30.8: oxazol-5-ylmethyl (4-((3- |
| | | azabicyclo[3.1.1]heptan-6-yl)methyl)phenyl)carbamate |
| | | LRMS (ES) m/z: 328.1[M + H]⁺. |
| | 9.1 | 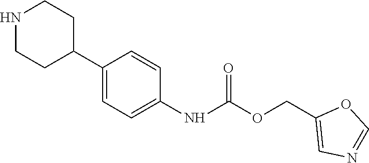 [SMILES: O=C(Nc1ccc(C2CCNCC2)cc1)OCc1cnco1] |
| | | Intermediate 30.9: oxazol-5-ylmethyl (4-(piperidin-4- |
| | | yl)phenyl)carbamate |
| | | LRMS (ES) m/z: 302.1[M + H]⁺. |
| | 8.1 | 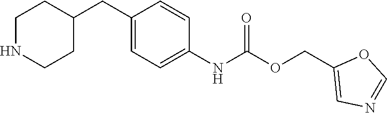 [SMILES: O=C(Nc1ccc(CC2CCNCC2)cc1)OCc1cnco1] |
| | | Intermediate 30.10: oxazol-5-ylmethyl (4-(piperidin-4- |
| | | ylmethyl)phenyl)carbamate hydrochloride |
| | | LRMS (ES) m/z: 316.1[M + H]⁺. |
| | 29.15 | 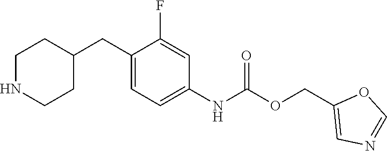 [SMILES: O=C(Nc1ccc(CC2CCNCC2)c(F)c1)OCc1cnco1] |
| | | Intermediate 30.10: oxazol-5-ylmethyl (3-fluoro-4-(piperidin-4- |
| | | ylmethyl)phenyl)carbamate hydrochloride |
| | | LRMS (ES) m/z: 334.1[M + H]⁺. |
| | 29.16 | 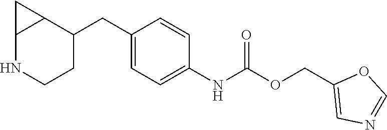 [SMILES: O=C(Nc1ccc(CC2CCNC3CC23)cc1)OCc1cnco1] |
| | | Intermediate 30.11: oxazol-5-ylmethyl (4-((2- |
| | | azabicyclo[4.1.0]heptan-5-yl)methyl)phenyl)carbamate |
| | | hydrochloride |
| | | LRMS (ES) m/z: 328.1[M + H]⁺. |
| | 29.17 | 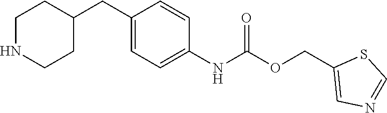 [SMILES: O=C(Nc1ccc(CC2CCNCC2)cc1)OCc1cncs1] |
| | | Intermediate 30.12: thiazol-5-ylmethyl (4-(piperidin-4- |
| | | ylmethyl)phenyl)carbamate. |
| | | LRMS (ES) m/z: 332.1[M + H]⁺. |

### Amine Derivatization

### Example L

### Method A: Synthesis of 4-chlorobenzyl (4-((1-(oxetan-3-yl)piperidin-4-yl)methyl)phenyl)carbamate (Compound 43)

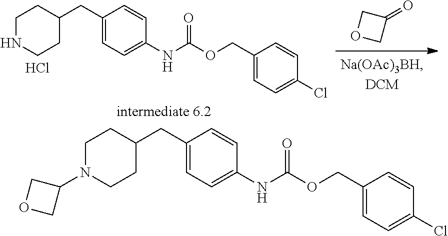 [SMILES: O=C(Nc1ccc(CC2CCNCC2)cc1)OCc1ccc(Cl)cc1 ; Cl ; O=C1COC1 ; O=C(Nc1ccc(CC2CCN(C3COC3)CC2)cc1)OCc1ccc(Cl)cc1]

To a mixture of 4-chlorobenzyl (4-(piperidin-4-ylmethyl)phenyl)carbamate hydrochloride (79 mg, 0.20 mmol, 1.00 equiv), 3-oxetanone (29 mg, 0.40 mmol, 1.00 equiv) in DCM (1 mL) was added Na(OAc)₃BH (93 mg, 0.44 mmol, 2.20 equiv). The reaction mixture was stirred for 30 min, then concentrated under reduced pressure, purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford 4-chlorobenzyl (4-((1-(oxetan-3-yl)piperidin-4-yl)methyl)phenyl)carbamate (80 mg, 96%) as a white solid. LRMS (ES) m/z: 415 [M+H]⁺. ¹H NMR (400 MHz, Methanol-d₄) δ 7.39 (dd, J=15.4, 7.0 Hz, 6H), 7.11 (d, J=8.2 Hz, 2H), 5.17 (s, 2H), 4.77 (t, J=7.2 Hz, 2H), 4.69 (t, J=6.6 Hz, 2H), 3.92 (p, J=6.5 Hz, 1H), 3.12 (d, J=12.0 Hz, 2H), 2.57 (d, J=6.9 Hz, 2H), 2.44-2.25 (m, 2H), 1.81 (d, J=14.9 Hz, 3H), 1.41 (qd, J=12.8, 11.8, 3.7 Hz, 2H).

### Method B: Synthesis of 4-chlorobenzyl (4-((1-acetylpiperidin-4-yl)methyl)phenyl)carbamate (Compound 40)

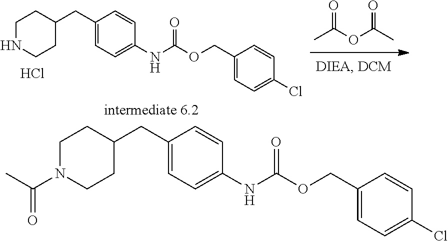

To a mixture of 4-chlorobenzyl (4-(piperidin-4-ylmethyl)phenyl)carbamate hydrochloride (0.20 g, 0.50 mmol, 1.00 equiv) in DCM (2 mL) was added DIEA (0.19 g, 1.50 mmol, 3.00 equiv), followed by acetic anhydride (0.10 g, 1.00 mmol, 2.00 equiv). The reaction mixture was stirred for 30 min, then concentrated under reduced pressure, purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford 4-chlorobenzyl (4-((1-acetylpiperidin-4-yl)methyl)phenyl)carbamate (138 mg, 69%) as a white solid. LRMS (ES) m/z: 401[M+H]⁺. ¹H NMR (400 MHz, Methanol-d₄) δ 7.48-7.28 (m, 6H), 7.10 (d, J=8.5 Hz, 2H), 5.17 (s, 2H), 4.49 (ddt, J=13.4, 4.6, 2.4 Hz, 1H), 3.89 (dp, J=13.6, 2.3 Hz, 1H), 3.04 (td, J=13.0, 2.7 Hz, 1H), 2.66-2.46 (m, 3H), 2.08 (s, 3H), 1.94-1.63 (m, 3H), 1.14 (dqd, J=37.1, 12.6, 4.3 Hz, 2H).

### Method C. Synthesis of 4-chlorobenzyl (4-((1-isobutyrylpiperidin-4-yl)methyl)phenyl)carbamate (Compound 87)

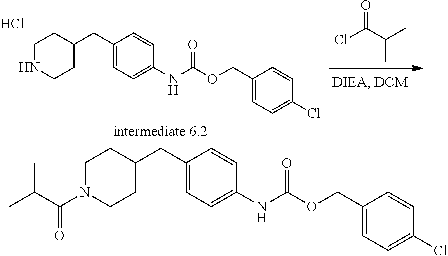

Followed the same procedure as above. Obtained the desired product 4-chlorobenzyl (4-((1-isobutyrylpiperidin-4-yl)methyl)phenyl)carbamate as a white solid (30 mg, 70%). LRMS (ES) m/z: 429 [M+H]⁺. ¹H NMR (400 MHz, Methanol-d₄) δ 7.53-7.20 (m, 6H), 7.10 (d, J=8.2 Hz, 2H), 5.17 (s, 2H), 4.52 (d, J=13.4 Hz, 1H), 4.03 (d, J=13.9 Hz, 1H), 3.13-2.79 (m, 2H), 2.68-2.42 (m, 3H), 1.96-1.57 (m, 3H), 1.23-0.96 (m, 8H).

### Method D. Synthesis of methyl 4-(4-((((4-chlorobenzyl)oxy)carbonyl)amino)benzyl)piperidine-1-carboxylate (Compound 98)

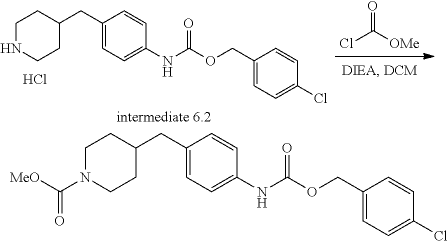 [SMILES: O=C(Nc1ccc(CC2CCNCC2)cc1)OCc1ccc(Cl)cc1 ; Cl ; COC(=O)Cl ; COC(=O)N1CCC(Cc2ccc(NC(=O)OCc3ccc(Cl)cc3)cc2)CC1]

To a mixture of 4-chlorobenzyl (4-(piperidin-4-ylmethyl)phenyl)carbamate hydrochloride (79 mg, 0.20 mmol, 1.00 equiv) in DCM (2 mL) was added DIEA (52 mg, 0.40 mmol, 2.00 equiv), followed by methyl chloroformate (19 mg, 0.20 mmol, 1.00 equiv). The reaction mixture was stirred for 30 min, then concentrated under reduced pressure, purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford methyl 4-(4-((((4-chlorobenzyl)oxy)carbonyl)amino)benzyl)piperidine-1-carboxylate (76 mg, 91%) as a white solid. LRMS (ES) m/z: 417 [M+H]⁺. ¹H NMR (400 MHz, Methanol-d₄) δ 7.53-7.23 (m, 6H), 7.21-6.97 (m, 2H), 5.17 (s, 2H), 4.08 (d, J=13.1 Hz, 2H), 3.67 (s, 3H), 2.76 (s, 2H), 2.51 (d, J=7.1 Hz, 2H), 1.64 (d, J=13.7 Hz, 3H), 1.12 (qd, J=12.6, 4.4 Hz, 2H).

### Method E. Synthesis of 4-chlorobenzyl (4-((1-(methylsulfonyl)piperidin-4-yl)methyl)phenyl)carbamate (Compound 38)

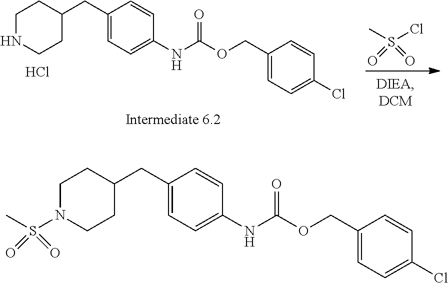

Followed the same procedure as above. Obtained the desired product 4-chlorobenzyl (4-((1-(methylsulfonyl) piperidin-4-yl)methyl)phenyl)carbamate (60 mg, 69%) as a white solid. LRMS (ES) m/z: 437 [M+H]⁺. ¹H NMR (400 MHz, Methanol-d₄) δ 7.39 (dq, J=11.5, 8.3, 7.9 Hz, 6H), 7.22-6.98 (m, 2H), 5.17 (s, 2H), 3.70 (d, J=11.5 Hz, 2H), 2.80 (s, 3H), 2.68 (td, J=12.0, 2.5 Hz, 2H), 2.55 (d, J=7.1 Hz, 2H), 1.75 (d, J=14.0 Hz, 3H), 1.38-1.16 (m, 2H).

### Method F. Synthesis of 4-chlorobenzyl (4-((1-(N,N-dimethylsulfamoyl)piperidin-4-yl)methyl)phenyl)carbamate (Compound 34

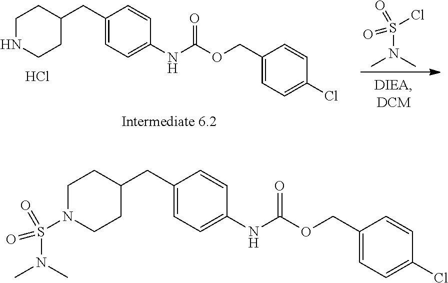

Followed the same procedure as above. Obtained the desired product 4-chlorobenzyl (4-((1-(N,N-dimethylsulfamoyl)piperidin-4-yl)methyl)phenyl)carbamate (77 mg, 83%) as a white solid. LRMS (ES) m/z: 466 [M+H]⁺. ¹H NMR (400 MHz, Methanol-d₄) δ 7.49-7.24 (m, 6H), 7.20-7.04 (m, 2H), 5.17 (s, 2H), 3.65 (dp, J=12.4, 1.9 Hz, 2H), 2.79 (s, 8H), 2.53 (d, J=6.7 Hz, 2H), 1.76-1.51 (m, 3H), 1.34-1.12 (m, 2H).

### Method G. Synthesis of 4-chlorobenzyl (4-((1-(dimethylcarbamoyl)piperidin-4-yl)methyl)phenyl)carbamate (Compound 58)

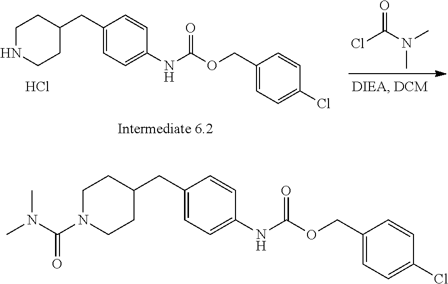

Followed the same procedure as above. Obtained the desired product 4-chlorobenzyl (4-((1-(dimethylcarbamoyl)piperidin-4-yl)methyl)phenyl)carbamate as a white solid (30 mg, 70%). LRMS (ES) m/z: 430 [M+H]⁺. ¹H NMR (400 MHz, Methanol-d₄) δ 7.52-7.26 (m, 6H), 7.10 (d, J=8.2 Hz, 2H), 5.17 (s, 2H), 3.65 (d, J=13.1 Hz, 2H), 2.83 (d, J=1.2 Hz, 6H), 2.73 (td, J=12.7, 2.3 Hz, 2H), 2.53 (d, J=6.9 Hz, 2H), 1.81-1.53 (m, 3H), 1.21 (qd, J=12.3, 4.0 Hz, 2H).

### Method H. Synthesis of oxazol-5-ylmethyl (4-((1-(2,2-difluoroethyl)piperidin-4-yl)methyl)phenyl)carbamate (Compound 59)

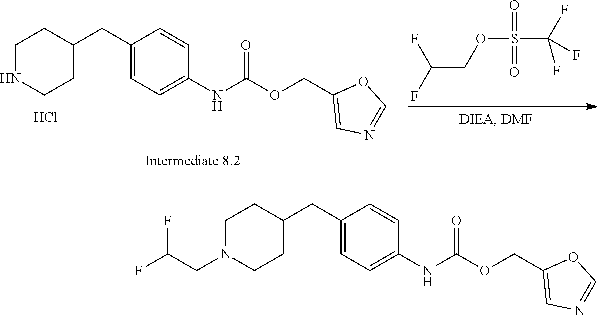

To a solution of oxazol-5-ylmethyl (4-(piperidin-4-ylmethyl)phenyl)carbamate hydrochloride (70 mg, 0.20 mmol, 1.00 equiv), DIEA (77 mg, 0.60 mmol, 3.00 equiv) in DMF (2 mL), was added 2,2-difluoroethyl trifluoromethanesulfonate (86 mg, 0.40 mmol, 2.00 equiv). The reaction mixture was stirred at rt for 4 h. The mixture was purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford the desired product oxazol-5-ylmethyl (4-((1-(2,2-difluoro-213-ethyl)piperidin-4-yl)methyl)phenyl)carbamate as a white solid (35 mg, 46%). LRMS (ES) m/z: 380 [M+H]⁺. ¹H NMR (400 MHz, Methanol-d₄) δ 8.23 (d, J=2.1 Hz, 1H), 7.35 (d, J=7.9 Hz, 2H), 7.25 (d, J=2.0 Hz, 1H), 7.15-6.98 (m, 2H), 6.09 (t, J=56 Hz, 1H), 5.24 (d, J=2.2 Hz, 2H), 3.12 (d, J=11.7 Hz, 2H), 2.96 (tt, J=15.4, 2.9 Hz, 2H), 2.52 (d, J=6.9 Hz, 2H), 2.40 (t, J=12.0 Hz, 2H), 1.81-1.49 (m, 3H), 1.37 (q, J=12.0 Hz, 2H).

### Method I. Preparation of (4-chlorophenyl)methyl N-(4-{[4-(pyrazin-2-yl)piperazin-1-yl]methyl}phenyl)carbamate (Compound 24)

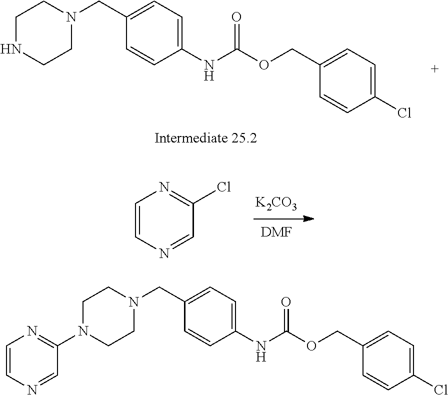

A mixture of (4-chlorophenyl)methyl N-[4-(piperazin-1-ylmethyl)phenyl]carbamate; trifluoroacetic acid (0.17 g, 0.348 mmol), 2-chloropyrazine (0.08 g, 0.70 mmol) and potassium carbonate (0.24 g, 1.74 mmol) in 1 ml DMF was stirred at 80 deg. After 18 h the mixture was diluted with EtOAc, washed with H₂O, dried (Na₂SO₄), concentrated and purified by prep HPLC (5-70% ACN/H₂O) afforded a clear foam (44 mg, 0.10 mmol, 29%). LCMS-ES (Pos) m/z: 438 [M+H]⁺. ¹H NMR (400 MHz, DMSO-d₆) δ 9.78 (s, 1H), 8.30 (d, J=1.4 Hz, 1H), 8.18-7.91 (m, 2H), 7.83 (d, J=2.6 Hz, 1H), 7.44 (m, 5H), 7.24 (d, J=8.3 Hz, 2H), 5.15 (s, 2H), 3.68-3.01 (m, 9H), 2.45 (m, 3H).

### Method J. Synthesis of 4-chlorobenzyl (4-((1-carbamoylpiperidin-4-yl)methyl)phenyl)carbamate (Compound 62)

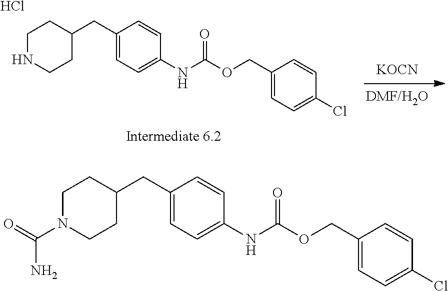

To a mixture of 4-chlorobenzyl (4-(piperidin-4-ylmethyl)phenyl)carbamate hydrochloride (79 mg, 0.20 mmol, 1.00 equiv), in DMF (1 mL) and H₂O (1 mL) was added potassium cyanate (49 mg, 0.60 mmol, 3.00 equiv). The reaction mixture was stirred at 60° C. for 16 h. H₂O (10 mL) was added to the mixture. The precipitate was filtered and washed with H₂O (5 mL), then dried over high vacuum to afford the desired product 4-chlorobenzyl (4-((1-carbamoylpiperidin-4-yl)methyl)phenyl)carbamate (55 mg, 68%) as a white solid. LRMS (ES) m/z: 402 [M+H]⁺. ¹H NMR (400 MHz, DMSO-d₆) δ 9.69 (s, 1H), 7.46 (s, 4H), 7.36 (d, J=8.0 Hz, 2H), 7.07 (d, J=8.1 Hz, 2H), 5.82 (s, 2H), 5.14 (s, 2H), 3.88 (d, J=13.1 Hz, 2H), 2.57 (d, J=12.5 Hz, 1H), 2.43 (d, J=7.0 Hz, 3H), 1.76-1.39 (m, 3H), 1.15-0.76 (m, 2H).

### Method K. Synthesis of oxazol-5-ylmethyl (4-(2-(1-(oxetane-3-carbonyl)piperidin-4-yl)ethyl)phenyl)carbamate (Compound 13)

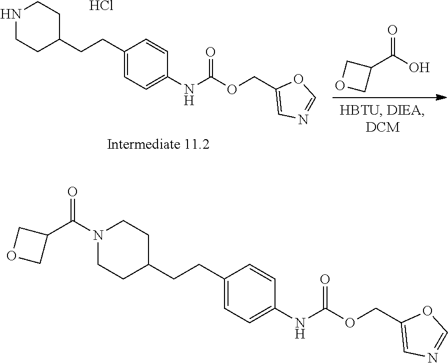

To a solution of oxetane-3-carboxylic acid (20 mg, 0.20 mmol, 2.00 equiv) in DMF (1 mL) was added HBTU (57 mg, 0.15 mmol, 1.50 equiv), DIEA (30 mg, 0.23 mmol, 2.30 equiv). The mixture was stirred for 10 min. oxazol-5-ylmethyl (4-(2-(piperidin-4-yl)ethyl)phenyl)carbamate hydrochloride (37 mg, 0.12 mmol, 1.0 equiv) was added. The reaction mixture was stirred for 30 min. The mixture was purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford the desired product oxazol-5-ylmethyl (4-(2-(1-(oxetane-3-carbonyl)piperidin-4-yl)ethyl)phenyl)carbamate as a white solid (20 mg, 48%). LRMS (ES) m/z: 414 [M+H]⁺. ¹H NMR (400 MHz, Methanol-d₄) δ 8.23 (s, 1H), 7.34 (d, J=8.1 Hz, 2H), 7.25 (s, 1H), 7.20-7.07 (m, 2H), 5.24 (s, 2H), 4.85-4.70 (m, 4H), 4.51 (ddt, J=13.2, 4.6, 2.5 Hz, 1H), 4.16 (h, J=7.7 Hz, 1H), 3.44 (ddt, J=13.6, 4.5, 2.4 Hz, 1H), 2.96 (ddd, J=13.6, 12.5, 2.8 Hz, 1H), 2.72-2.54 (m, 3H), 1.80 (ddt, J=13.2, 8.1, 2.2 Hz, 2H), 1.68-1.45 (m, 3H), 1.18-1.01 (m, 2H).

Compounds in the following table were prepared in a similar manner as above example compounds, using the intermediates with alkylation/acylation reagents and methods as listed.

| | | Alkylation/ | |
|---|---|---|---|
| | | acylation | |
| | Inter- | Reagent | |
| Co# | mediate | Method | Structure, Name and Data |
| 17 | 6.2 | MeNHSO₂Cl F | 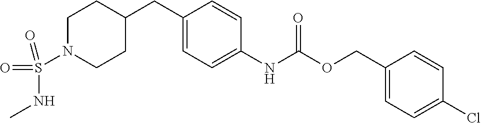 [SMILES: CNS(=O)(=O)N1CCC(Cc2ccc(NC(=O)OCc3ccc(Cl)cc3)cc2)CC1] |
| | | | Compound 17: 4-chlorobenzyl (4-((1-(N- |
| | | | methylsulfamoyl)piperidin-4- |
| | | | yl)methyl)phenyl)carbamate LRMS (ES) m/z: 452 |
| | | | [M + H]⁺. ¹H NMR (400 MHz, Methanol-d₄) δ 7.46-7.32 |
| | | | (m, 6H), 7.14-7.05 (m, 2H), 5.16 (s, 2H), 3.62 (dt, J = 12.1, |
| | | | 2.6 Hz, 2H), 2.69 (td, J = 12.2, 2.4 Hz, 2H), 2.62 (s, 3H), |
| | | | 2.52 (d, J = 6.9 Hz, 2H), 1.76-1.54 (m, 3H), 1.36-1.14 |
| | | | (m, 2H). |
| 102 | 6.2 | c-BuCOCl C | 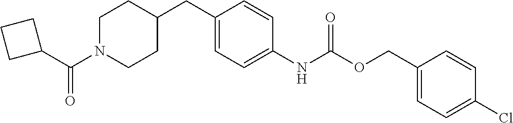 [SMILES: O=C(Nc1ccc(CC2CCN(C(=O)C3CCC3)CC2)cc1)OCc1ccc(Cl)cc1] |
| | | | Compound 102: 4-chlorobenzyl (4-((1- |
| | | | (cyclobutanecarbonyl)piperidin-4- |
| | | | yl)methyl)phenyl)carbamate. LRMS (ES) m/z: 441 |
| | | | [M + H]⁺. ¹H NMR (400 MHz, Methanol-d₄) δ 7.56-7.26 |
| | | | (m, 6H), 7.10 (d, J = 8.1 Hz, 2H), 5.17 (s, 2H), 4.47 (d, J = |
| | | | 13.3 Hz, 1H), 3.85-3.67 (m, 1H), 3.46-3.35 (m, 1H), 2.94 |
| | | | (t, J = 12.4 Hz, 1H), 2.68-2.48 (m, 3H), 2.41-2.10 (m, |
| | | | 4H), 2.10-1.91 (m, 1H), 1.91-1.56 (m, 4H), 1.16-1.03 |
| | | | (m, 2H). |
| 313 | 7.2 | oxetan-3-one A | 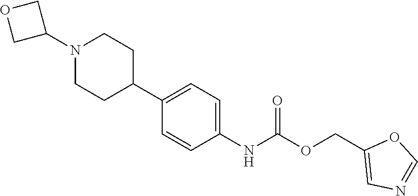 [SMILES: O=C(Nc1ccc(C2CCN(C3COC3)CC2)cc1)OCc1cnco1] |
| | | | Compound 313. 1,3-oxazol-5-ylmethyl N-{4-[1-(oxetan- |
| | | | 3-yl)piperidin-4-yl]phenyl}carbamate. LCMS-ES (Pos) |
| | | | m/z: 358 [M + H]⁺. ¹H NMR (400 MHz, DMSO-d₆) δ 9.73 |
| | | | (s, 1H), 8.43 (s, 1H), 7.40-7.28 (m, 3H), 7.16 (d, J = 8.1 |
| | | | Hz, 2H), 5.21 (s, 2H), 4.54 (t, J = 6.5 Hz, 2H), 4.44 (t, J = |
| | | | 6.1 Hz, 2H), 3.39 (t, J = 6.4 Hz, 1H), 2.78 (d, J = 10.9 Hz, |
| | | | 2H), 2.43 (t, J = 12.0 Hz, 1H), 1.83 (t, J = 11.3 Hz, 2H), |
| | | | 1.76-1.67 (m, 1H), 1.70 (s, 1H), 1.63 (td, J = 11.9, 11.4, |
| | | | 3.3 Hz, 2H). |
| 15 | 7.2 | Ac₂O B | 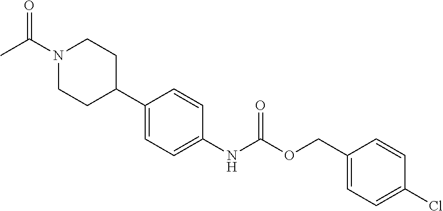 [SMILES: CC(=O)N1CCC(c2ccc(NC(=O)OCc3ccc(Cl)cc3)cc2)CC1] |
| | | | Compound 15: (4-chlorophenyl)methyl N-[4-(1- |
| | | | acetylpiperidin-4-yl)phenyl]carbamate LCMS-APCI |
| | | | (POS.) m/z: 387.1 [M + H]⁺. ¹H NMR (400 MHz, Methanol- |
| | | | d₄) δ 7.44-7.25 (m, 6H), 7.14 (d, J = 8.3 Hz, 2H), 5.14 (s, |
| | | | 2H), 4.68-4.60 (m, 1H), 3.98 (m, 1H), 3.21 (td, J = 13.2, |
| | | | 2.6 Hz, 1H), 2.72 (m, 2H), 1.98-1.78 (m, 2H), 1.60 (m, |
| | | | 2H). |
| 198 | 7.2 | oxetan-3-one A | 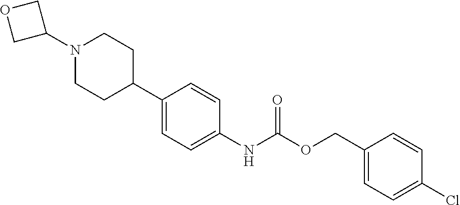 [SMILES: O=C(Nc1ccc(C2CCN(C3COC3)CC2)cc1)OCc1ccc(Cl)cc1] |
| | | | Compound 198: (4-chlorophenyl)methyl N-{4-[1- |
| | | | (oxetan-3-yl)piperidin-4-yl]phenyl}carbamate. LCMS- |
| | | | APCI (POS.) m/z: 447.1 [M + H]⁺. ¹H NMR (400 MHz, |
| | | | DMSO-d₆) δ 9.70 (s, 1H), 8.19 (s, 1H), 7.46 (s, 4H), 7.38 |
| | | | (d, J = 8.2 Hz, 2H), 7.24-7.05 (m, 2H), 5.13 (s, 2H), 4.54 |
| | | | (t, J = 6.5 Hz, 2H), 4.44 (t, J = 6.1 Hz, 2H), 3.39 (p, J = 6.4 |
| | | | Hz, 1H), 2.78 (d, J = 10.9 Hz, 2H), 2.43 (ddt, J = 11.9, 7.7, |
| | | | 3.9 Hz, 1H), 1.83 (td, J = 11.6, 2.4 Hz, 2H), 1.77-1.51 (m, |
| | | | 4H). |
| 31 | 8.2 | MeCOCl C | 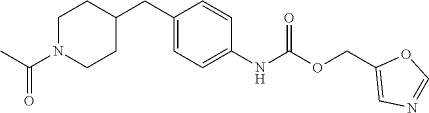 [SMILES: CC(=O)N1CCC(Cc2ccc(NC(=O)OCc3cnco3)cc2)CC1] |
| | | | Compound 31: oxazol-5-ylmethyl (4-((1- |
| | | | acetylpiperidin-4-yl)methyl)phenyl)carbamate LRMS |
| | | | (ES) m/z: 359 [M + H]⁺. ¹H NMR (400 MHz, Methanol-d₄) |
| | | | δ 8.24 (s, 1H), 7.35 (d, J = 8.0 Hz, 2H), 7.25 (s, 1H), 7.11 |
| | | | (d, J = 8.2 Hz, 2H), 5.25 (s, 2H), 4.49 (dq, J = 14.2, 2.6 Hz, |
| | | | 1H), 3.94-3.78 (m, 1H), 3.10-2.94 (m, 1H), 2.66-2.42 |
| | | | (m, 3H), 2.09 (s, 3H), 1.88-1.62 (m, 3H), 1.26-1.02 (m, |
| | | | 2H) |
| 35 | 8.2 | i-PrCOCl C | 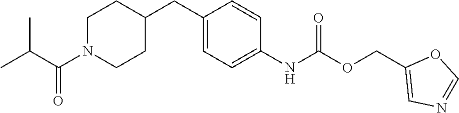 [SMILES: CC(C)C(=O)N1CCC(Cc2ccc(NC(=O)OCc3cnco3)cc2)CC1] |
| | | | Compound 35: oxazol-5-ylmethyl (4-((1- |
| | | | isobutyrylpiperidin-4-yl)methyl)phenyl)carbamate |
| | | | LRMS (ES) m/z: 386 [M + H]⁺. ¹H NMR (400 MHz, |
| | | | Methanol-d₄) δ 8.23 (s, 1H), 7.36 (d, J = 8.1 Hz, 2H), 7.25 |
| | | | (s, 1H), 7.10 (d, J = 8.2 Hz, 2H), 5.24 (s, 2H), 4.58-4.42 |
| | | | (m, 1H), 4.10-3.90 (m, 1H), 3.10-2.99 (m, 1H), 2.93 (h, |
| | | | J = 6.8 Hz, 1H), 2.67-2.36 (m, 3H), 1.91-1.59 (m, 3H), |
| | | | 1.29-0.92 (m, 9H). |
| 30 | 8.2 | Me₂NCOCl G | 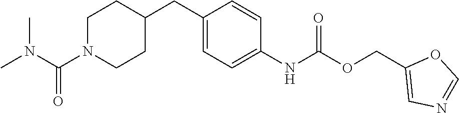 [SMILES: CN(C)C(=O)N1CCC(Cc2ccc(NC(=O)OCc3cnco3)cc2)CC1] |
| | | | Compound 30: oxazol-5-ylmethyl (4-((1- |
| | | | (dimethylcarbamoyl)piperidin-4- |
| | | | yl)methyl)phenyl)carbamate LRMS (ES) m/z: |
| | | | 387[M + H]⁺. ¹H NMR (400 MHz, Methanol-d₄) δ 8.24 (s, |
| | | | 1H), 7.35 (d, J = 8.0 Hz, 2H), 7.25 (s, 1H), 7.18-6.98 (m, |
| | | | 2H), 5.24 (s, 2H), 3.64 (d, J = 13.0 Hz, 2H), 2.82 (d, J = 1.4 |
| | | | Hz, 6H), 2.72 (td, J = 12.8, 2.3 Hz, 2H), 2.52 (d, J = 6.9 Hz, |
| | | | 2H), 1.76-1.56 (m, 3H), 1.28-1.12 (m, 2H). |
| 11 | 8.2 | MeOCOCl D | 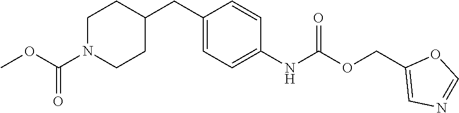 [SMILES: COC(=O)N1CCC(Cc2ccc(NC(=O)OCc3cnco3)cc2)CC1] |
| | | | Compound 11: methyl 4-(4-(((oxazol-5- |
| | | | ylmethoxy)carbonyl)amino)benzyl)piperidine-1- |
| | | | carboxylate LRMS (ES) m/z: 374 [M + H]⁺. ¹H NMR |
| | | | (400 MHz, Methanol-d₄) δ 8.24 (s, 1H), 7.35 (d, J = 8.0 |
| | | | Hz, 2H), 7.25 (s, 1H), 7.10 (d, J = 8.2 Hz, 2H), 5.25 (s, |
| | | | 2H), 4.08 (d, J = 13.1 Hz, 2H), 3.68 (s, 3H), 2.86-1.67 |
| | | | (m, 2H), 2.52 (d, J = 7.0 Hz, 2H), 1.81-1.51 (m, 3H), |
| | | | 1.22-1.05 (m, 2H). |
| 18 | 8.2 | MeSO₂Cl E | 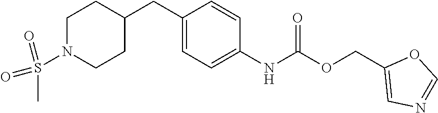 [SMILES: CS(=O)(=O)N1CCC(Cc2ccc(NC(=O)OCc3cnco3)cc2)CC1] |
| | | | Compound 18: oxazol-5-ylmethyl (4-((1- |
| | | | (methylsulfonyl)piperidin-4- |
| | | | yl)methyl)phenyl)carbamate LRMS (ES) m/z: 394 |
| | | | [M + H]⁺. ¹H NMR (400 MHz, Methanol-d₄) δ 8.24 (s, 1H), |
| | | | 7.36 (d, J = 8.0 Hz, 2H), 7.25 (s, 1H), 7.11 (d, J = 8.1 Hz, |
| | | | 2H), 5.24 (s, 2H), 3.69 (d, J = 11.2 Hz, 2H), 2.80 (s, 3H), |
| | | | 2.66 (td, J = 12.1, 2.5 Hz, 2H), 2.54 (d, J = 7.1 Hz, 2H), |
| | | | 1.84-1.52 (m, 3H), 1.38-1.20 (m, 2H). |
| 10 | 8.2 | MeNHSO₂Cl F | 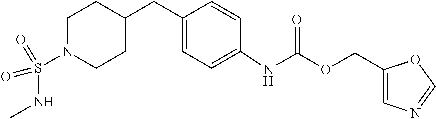 [SMILES: CNS(=O)(=O)N1CCC(Cc2ccc(NC(=O)OCc3cnco3)cc2)CC1] |
| | | | Compound 10: oxazol-5-ylmethyl (4-((1-(N- |
| | | | methylsulfamoyl)piperidin-4- |
| | | | yl)methyl)phenyl)carbamate. LRMS (ES) m/z: 409 |
| | | | [M + H]⁺. ¹H NMR (400 MHz, Methanol-d₄) δ 8.23 (s, 1H), |
| | | | 7.35 (d, J = 8.0 Hz, 2H), 7.25 (s, 1H), 7.10 (d, J = 8.2 Hz, |
| | | | 2H), 5.24 (s, 2H), 3.62 (d, J = 12.3 Hz, 2H), 2.69 (td, J = |
| | | | 12.2, 2.3 Hz, 2H), 2.62 (s, 3H), 2.52 (d, J = 6.9 Hz, 2H), |
| | | | 1.74-1.50 (m, 3H), 1.35-1.19 (m, 2H). |
| 27 | 8.2 | Me₂NSO₂Cl F | 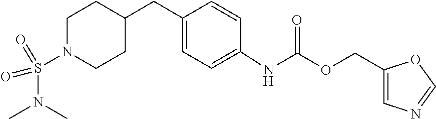 [SMILES: CN(C)S(=O)(=O)N1CCC(Cc2ccc(NC(=O)OCc3cnco3)cc2)CC1] |
| | | | Compound 27: oxazol-5-ylmethyl (4-((1-(N,N- |
| | | | dimethylsulfamoyl)piperidin-4- |
| | | | yl)methyl)phenyl)carbamate LRMS (ES) m/z: 409 |
| | | | [M + H]⁺. ¹H NMR (400 MHz, Methanol-d₄) δ 8.23 (s, 1H), |
| | | | 7.35 (d, J = 8.0 Hz, 2H), 7.25 (s, 1H), 7.14-6.96 (m, 2H), |
| | | | 5.24 (s, 2H), 3.64 (dq, J = 12.5, 2.4 Hz, 2H), 2.79 (s, |
| | | | 6H),2.80-2.69 (m, 2H), 2.52 (d, J = 6.7 Hz, 2H), 1.77- |
| | | | 1.47 (m, 3H), 1.42-1.04 (m, 2H). |
| 80 | 8.2 | oxetan-3-one A | 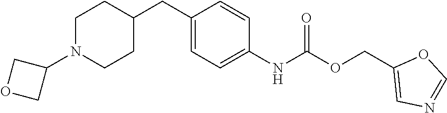 [SMILES: O=C(Nc1ccc(CC2CCN(C3COC3)CC2)cc1)OCc1cnco1] |
| | | | Compound 80: oxazol-5-ylmethyl (4-((1-(oxetan-3- |
| | | | yl)piperidin-4-yl)methyl)phenyl)carbamate LRMS (ES) |
| | | | m/z: 372 [M + H]⁺. ¹H NMR (400 MHz, Methanol-d₄) δ |
| | | | 8.36 (s, 1H), 8.24 (s, 1H), 7.36 (d, J = 8.0 Hz, 2H), 7.25 (s, |
| | | | 1H), 7.16-7.02 (m, 2H), 5.25 (s 2H), 4.75 (t, J = 7.1 Hz, |
| | | | 2H), 4.69 (t, J = 6.7 Hz, 2H), 3.86 (p, J = 6.7 Hz, 1H), |
| | | | 3.07 (d, J = 11.8 Hz, 2H), 2.56 (d, J = 6.9 Hz, 2H), 2.26 (t, |
| | | | J = 12.0 Hz, 2H), 1.86-1.60 (m, 2H), 1.50-1.26 (m, 2H). |
| 48 | 8.2 | oxetane-3- carbonyl chloride C | 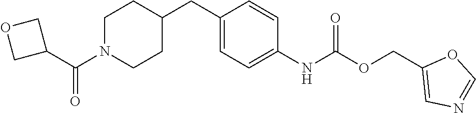 [SMILES: O=C(Nc1ccc(CC2CCN(C(=O)C3COC3)CC2)cc1)OCc1cnco1] |
| | | | Compound 48: oxazol-5-ylmethyl (4-((1-(oxetane-3- |
| | | | carbonyl)piperidin-4-yl)methyl)phenyl)carbamate |
| | | | LRMS (ES) m/z: 400 [M + H]⁺. ¹H NMR (400 MHz, |
| | | | Methanol-d₄) δ 8.24 (s, 1H), 7.35 (d, J = 7.9 Hz, 2H), 7.25 |
| | | | (s, 1H), 7.17-7.00 (m, 2H), 5.24 (s, 2H), 4.86-4.73 (m, |
| | | | 4H), 4.49 (dq, J = 13.0, 2.2 Hz, 1H), 4.15 (p, J = 7.8 Hz, |
| | | | 1H), 3.47-3.36 (m, 1H), 2.93 (td, J = 13.1, 2.5 Hz, 1H), |
| | | | 2.61 (td, J = 12.9, 2.7 Hz, 1H), 2.55-2.34 (m, 2H), 1.90- |
| | | | 1.55 (m, 3H), 1.20-0.99 (m, 2H). |
| 293 | 8.2 | MeOCH₂CH₂Br H | 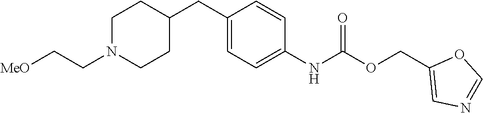 [SMILES: COCCN1CCC(Cc2ccc(NC(=O)OCc3cnco3)cc2)CC1] |
| | | | Compound 293: oxazol-5-ylmethyl (4-((1-(2- |
| | | | methoxyethyl)piperidin-4-yl)methyl)phenyl)carbamate |
| | | | LRMS (ES) m/z: 374 [M + H]⁺. ¹H NMR (400 MHz, |
| | | | Methanol-d₄) δ 8.24 (d, J = 2.2 Hz, 1H), 7.37 (d, J = 8.0 Hz, |
| | | | 2H), 7.25 (s, 1H), 7.12 (d, J = 8.0 Hz, 2H), 5.25 (s, 2H), |
| | | | 3.67 (s, 2H), 3.51-3.38 (m, 2H), 3.40 (s, 3H), 3.15 (t, J = |
| | | | 5.2 Hz, 2H), 2.78 (t, J = 12.5 Hz, 2H), 2.57 (d, J = 6.4 Hz, |
| | | | 2H), 1.88-1.73 (m, 3H), 1.58-1.41 (m, 2H). |
| 70 | 8.2 | F₂CHCH₂OTf H | 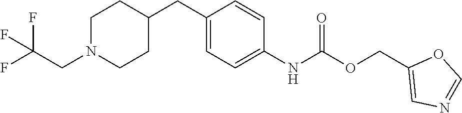 [SMILES: O=C(Nc1ccc(CC2CCN(CC(F)(F)F)CC2)cc1)OCc1cnco1] |
| | | | Compound 70: oxazol-5-ylmethyl (4-((1-(2,2,2- |
| | | | trifluoroethyl)piperidin-4-yl)methyl)phenyl)carbamate |
| | | | LRMS (ES) m/z: 398 [M + H]⁺. ¹H NMR (400 MHz, |
| | | | Methanol-d₄) δ 8.24(s, 1H), 7.34 (d, J = 8.0 Hz, 2H), 7.25 |
| | | | (d, J = 2.2 Hz, 1H), 7.18-6.98 (m, 2H), 5.24 (s, 2H), 3.10- |
| | | | 2.90 (m, 4H), 2.50 (dd, J = 7.0, 2.2 Hz, 2H), 2.29 (t, J = |
| | | | 11.7 Hz, 2H), 1.68-1.40 (m, 3H), 1.38-1.22 (m, 2H). |
| 115 | 10.2 | MeOCOCl D | 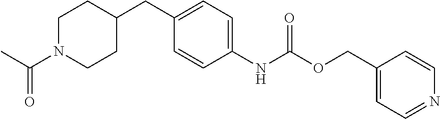 [SMILES: CC(=O)N1CCC(Cc2ccc(NC(=O)OCc3ccncc3)cc2)CC1] |
| | | | Compound 115: pyridin-4-ylmethyl (4-((1- |
| | | | acetylpiperidin-4-yl)methyl)phenyl)carbamate. LCMS- |
| | | | ES (Pos) m/z: 368.1 [M + H]⁺. ¹H NMR (400 MHz, DMSO- |
| | | | d₆) δ 9.80 (s, 1H), 8.58 (d, J = 4.9 Hz, 2H), 7.41-7.34 (m, |
| | | | 4H), 7.08 (d, J = 7.9 Hz, 2H), 5.19 (s, 2H), 4.32 (d, J = 13.2 |
| | | | Hz, 1H), 3.75 (d, J = 13.6 Hz, 1H), 3.22-3.12 (m, 1H), |
| | | | 2.92 (t, J = 12.9 Hz, 1H), 2.44 (d, J = 8.0 Hz, 2H), 1.95 (s, |
| | | | 3H), 1.75-1.63 (m, 1H), 1.55 (t, J = 13.0 Hz, 2H), 1.14- |
| | | | 1.00 (m, 1H), 1.00-0.88 (m, 1H). |
| 23 | 10.2 | MeOCOCl D | 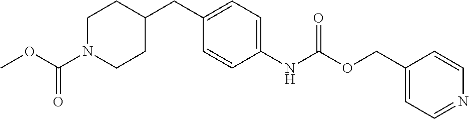 [SMILES: COC(=O)N1CCC(Cc2ccc(NC(=O)OCc3ccncc3)cc2)CC1] |
| | | | Compound 23: methyl 4-(4-(((pyridin-4- |
| | | | ylmethoxy)carbonyl)amino)benzyl)piperidine-1- |
| | | | carboxylate. LCMS-ES (Pos) m/z: 384.1 [M + H]⁺. ¹H |
| | | | NMR (400 MHz, Methanol-d₄) δ 8.53 (d, J = 3.4 Hz 2H), |
| | | | 7.47 (d, J = 5.0 Hz, 2H), 7.36 (d, J = 8.0 Hz, 2H), 7.09 (d, |
| | | | J = 8.5, Hz, 2H), 5.25 (s, 2H), 4.06 (d, J = 13.2 Hz, 2H), |
| | | | 3.66 (s, 3H), 2.83-2.65 (m, 2H), 2.50 (d, J = 7.1 Hz, 2H), |
| | | | 1.79-1.55 (m, 3H), 1.11 (qd, J = 12.3, 6.7 Hz, 2H). |
| 316 | 10.2 | 3-oxetanone A | 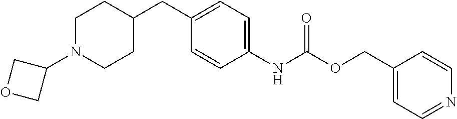 [SMILES: O=C(Nc1ccc(CC2CCN(C3COC3)CC2)cc1)OCc1ccncc1] |
| | | | Compound 316: 1,3-oxazol-5-ylmethyl N-[4-(piperidin- |
| | | | 4-yl)phenyl]carbamate. |
| | | | LCMS-ES (Pos) m/z: 382 [M + H]⁺. ¹H NMR (400 MHz, |
| | | | DMSO-d6) δ 9.80 (s, 1H), 8.58 (d, J = 4.9 Hz, 2H), 8.15 |
| | | | (s, 1H), 7.42-7.33 (m, 4H), 7.07 (d, J = 8.4 Hz, 2H), 5.20 |
| | | | (s, 2H), 4.49 (t, J = 6.7 Hz, 2H), 4.38 (t, J = 5.9 Hz, 2H), |
| | | | 3.70 (d, J = 9.5 Hz, 1H), 3.49 (d, J = 12.2 Hz, 1H), 2.64 |
| | | | (d, J = 11.1 Hz, 2H), 2.44 (d, J = 6.7 Hz, 1H), 1.65 (t, J = |
| | | | 11.4 Hz, 2H), 1.53 (d, J = 12.9 Hz, 2H), 1.18 (t, J = 11.4 |
| | | | Hz, 2H). |
| 52 | 10.2 | F₂CHCH₂OTf H | 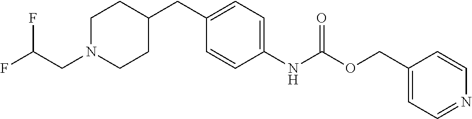 [SMILES: O=C(Nc1ccc(CC2CCN(CC(F)F)CC2)cc1)OCc1ccncc1] |
| | | | Compound 52: 1,3-oxazol-5-ylmethyl N-[4-(piperidin- |
| | | | 4-yl)phenyl]carbamate. LCMS-ES (Pos) m/z: 390 |
| | | | [M + H]⁺. ¹H NMR (400 MHz, DMSO-d₆) δ 12.68 (s, 1H), |
| | | | 9.72 (s, 1H), 8.42 (d, J = 2.0 Hz, 1H), 8.14 (d, J = 2.0 Hz, |
| | | | 1H), 7.33 (dd, J = 22.8, 5.0 Hz, 3H), 7.22-7.03 (m, 2H), |
| | | | 6.16 (s, 1H), 5.21 (s, 2H), 4.10 (s, 1H), 3.18 (s, 1H), 3.00 |
| | | | (d, J = 11.1 Hz, 2H), 2.91-2.63 (m, 2H), 2.42 (t, J = 12.1 |
| | | | Hz, 1H), 2.26 (t, J = 11.5 Hz, 2H), 1.96-1.52 (m, 4H). |
| 7 | 9.2 | i-PrCOCl C | 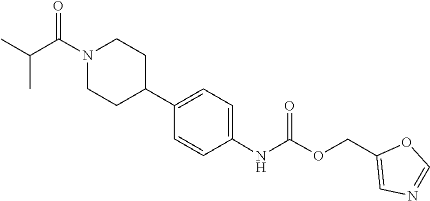 [SMILES: CC(C)C(=O)N1CCC(c2ccc(NC(=O)OCc3cnco3)cc2)CC1] |
| | | | Compound 7: 1,3-oxazol-5-ylmethyl N-{4-[1-(2- |
| | | | methylpropanoyl)piperidin-4-yl]phenyl}carbamate. |
| | | | LCMS-ES (Pos) m/z: 372.1 [M + H]⁺. ¹H NMR (400 MHz, |
| | | | DMSO-d₆) δ 9.73 (s, 1H), 8.42 (d, J = 2.0 Hz, 1H), 7.49- |
| | | | 7.26 (m, 2H), 7.16 (d, J = 8.1 Hz, 2H), 5.21 (s, 2H), 4.55 (d, |
| | | | J = 12.8 Hz, 1H), 4.16-3.91 (m, 1H), 3.09 (t, J = 13.0 Hz, |
| | | | 1H), 2.90 (p, J = 6.8 Hz, 1H), 2.71 (t, J = 12.3 Hz, 1H), 2.58 |
| | | | (d, J = 12.0 Hz, 2H), 1.78 (s, 2H), 1.60-1.25 (m, 2H), 1.25- |
| | | | 0.89 (m, 6H). |
| 8 | 9.2 | Ac₂O B | 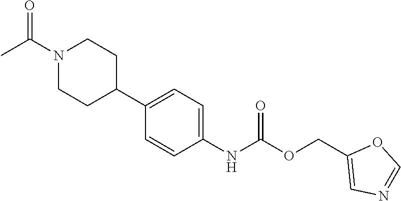 [SMILES: CC(=O)N1CCC(c2ccc(NC(=O)OCc3cnco3)cc2)CC1] |
| | | | Compound 8: 1,3-oxazol-5-ylmethyl N-[4-(1- |
| | | | acetylpiperidin-4-yl)phenyl]carbamate |
| | | | LCMS-ES (Pos) m/z: 344 [M + H]⁺. ¹H NMR (400 MHz, |
| | | | DMSO-d₆) δ 9.74 (s, 1H), 7.49-7.22 (m, 3H), 7.16 (d, J = |
| | | | 8.1 Hz, 2H), 5.21 (s, 2H), 4.64-4.39 (m, 1H), 3.90 (d, J = |
| | | | 13.6 Hz, 1H), 3.10 (t, J = 12.9 Hz, 1H), 2.69 (t, J = 12.2 Hz, |
| | | | 1H), 2.57 (d, J = 13.0 Hz, 2H), 2.02 (s, 3H), 1.74 (t, J = 13.8 |
| | | | Hz, 2H), 1.47 (dtt, J = 57.2, 12.5, 6.3 Hz, 2H). |
| 88 | 9.2 | F₃CCH₂OTf H | 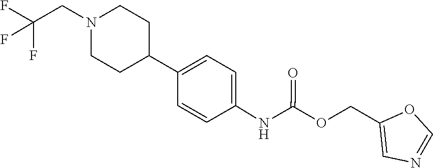 [SMILES: O=C(Nc1ccc(C2CCN(CC(F)(F)F)CC2)cc1)OCc1cnco1] |
| | | | Compound 88: 1,3-oxazol-5-ylmethyl N-{4-[1-(2,2,2- |
| | | | trifluoroethyl)piperidin-4-yl]phenyl}. LCMS-ES (Pos) |
| | | | m/z: 384 [M + H]⁺. ¹H NMR (400 MHz, DMSO-d₆) δ 9.73 |
| | | | (s, 1H), 8.42 (d, J = 2.0 Hz, 1H), 7.33 (dd, J = 23.5, 5.0 Hz, |
| | | | 3H), 7.21-6.90 (m, 2H), 5.21 (s, 2H), 3.11 (d, J = 74.1 Hz, |
| | | | 2H), 3.38 (m, 2H), 2.44 (s, 2H), 1.67 (d, J = 15.8 Hz, 4H). |
| 113 | 9.2 | F₂CHCH₂OTf H | 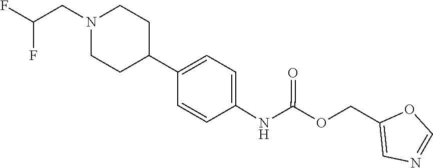 [SMILES: O=C(Nc1ccc(C2CCN(CC(F)F)CC2)cc1)OCc1cnco1] |
| | | | Compound 113: 1,3-oxazol-5-ylmethyl N-{4-[1-(2,2- |
| | | | difluoroethyl)piperidin-4-yl]phenyl}carbamate. LCMS- |
| | | | ES (Pos) m/z: 366 [M + H]⁺. ¹H NMR (400 MHz, DMSO- |
| | | | d₆) δ 9.72 (s, 1H), 8.42 (d, J = 2.0 Hz, 1H), 8.14 (d, J = 2.0 |
| | | | Hz, 1H), 7.33 (dd, J = 22.8, 5.0 Hz, 3H), 7.22-7.03 (m, |
| | | | 2H), 6.16 (s, 1H), 5.21 (s, 2H), 4.10 (s, 1H), 3.18 (s, 1H), |
| | | | 3.00 (d, J = 11.1 Hz, 2H), 2.91-2.63 (m, 1H), 2.42 (t, J = |
| | | | 12.1 Hz, 1H), 2.26 (t, J = 11.5 Hz, 2H), 1.96-1.52 (m, 2H). |
| 148 | 9.2 | MeOCOCl D | 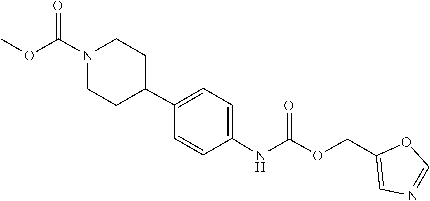 [SMILES: COC(=O)N1CCC(c2ccc(NC(=O)OCc3cnco3)cc2)CC1] |
| | | | Compound 148. methyl 4-(4-(((oxazol-5- |
| | | | ylmethoxy)carbonyl)amino)phenyl)piperidine-1- |
| | | | carboxylate LCMS-ES (Pos) m/z: 360 (M + H). ¹H NMR |
| | | | (400 MHz, DMSO-d₆) δ 9.74 (s, 1H), 8.43 (s, 1H), 7.43- |
| | | | 7.26 (m, 2H), 7.16 (d, J = 8.1 Hz, 2H), 5.21 (s, 2H), 4.09 (s, |
| | | | 2H), 3.61 (s, 3H), 2.86 (m, 3H), 2.64 (t, J = 12.3 Hz, 1H), |
| | | | 1.72 (d, J = 12.9 Hz, 2H), 1.47 (qd, J = 12.7, 4.2 Hz, 2H). |
| 4 | 9.2 | Me₂NCOCl G | 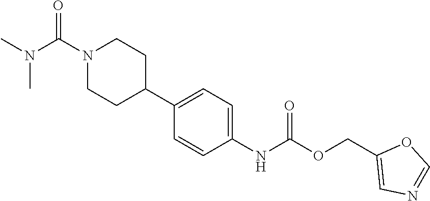 [SMILES: CN(C)C(=O)N1CCC(c2ccc(NC(=O)OCc3cnco3)cc2)CC1] |
| | | | Compound 4: oxazol-5-ylmethyl (4-(1- |
| | | | (dimethylcarbamoyl)piperidin-4-yl)phenyl)carbamate. |
| | | | LCMS-ES (Pos) m/z: 373 [M + H]⁺. ¹H NMR (400 MHz, |
| | | | DMSO-d₆) δ 9.73 (s, 1H), 8.42 (s, 1H), 7.36 (d, J = 8.1 Hz, |
| | | | 2H), 7.30 (s, 1H), 7.15 (d, J = 8.1 Hz, 2H), 5.20 (s, 2H), |
| | | | 3.63 (d, J = 12.7 Hz, 2H), 2.80-2.70 (m, 2H), 2.74 (s, 6H), |
| | | | 2.59 (t, J = 12.1 Hz, 1H), 1.75-1.67 (m, 2H), 1.54 (qd, J = |
| | | | 12.5, 3.5 Hz, 2H). |
| 348 | 9.2 | MeOCH₂CH₂Br H | 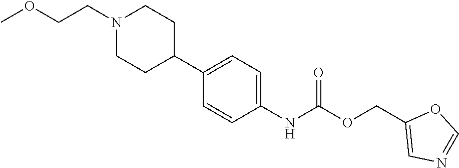 [SMILES: COCCN1CCC(c2ccc(NC(=O)OCc3cnco3)cc2)CC1] |
| | | | Compound 348: 1,3-oxazol-5-ylmethyl N-{4-[1-(2- |
| | | | methoxyethyl)piperidin-4-yl]phenyl}carbamate. |
| | | | LCMS-ES (Pos) m/z: 360 [M + H]⁺. ¹H NMR (400 MHz, |
| | | | DMSO-d₆) δ 9.74 (s, 1H), 8.43 (s, 1H), 7.37 (d, J = 8.1 Hz, |
| | | | 2H), 7.31 (s, 1H), 7.16 (d, J = 8.1 Hz, 2H), 5.21 (s, 2H), |
| | | | 3.64 (d, J = 12.7 Hz, 2H), 2.75 (s, 8H), 2.60 (t, J = 12.1 Hz, |
| | | | 1H), 1.72 (d, J = 12.2 Hz, 2H), 1.55 (m, 2H). |
| 26 | 11.2 | Ac₂O B | 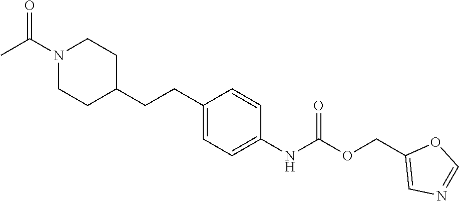 [SMILES: CC(=O)N1CCC(CCc2ccc(NC(=O)OCc3cnco3)cc2)CC1] |
| | | | Compound 26: oxazol-5-ylmethyl (4-(2-(1- |
| | | | acetylpiperidin-4-yl)ethyl)phenyl)carbamate LRMS |
| | | | (ES) m/z: 372 [M + H]⁺. ¹H NMR (400 MHz, Methanol-d₄) |
| | | | δ 8.24 (s, 1H), 7.34 (d, J = 8.2 Hz, 2H), 7.25 (s, 1H), 7.20- |
| | | | 7.01 (m, 2H), 5.24 (s, 2H), 4.50 (ddt, J = 13.1, 4.5, 2.5 |
| | | | Hz, 1H), 3.91 (ddt, J = 13.5, 4.5, 2.4 Hz, 1H), 3.17-3.00 |
| | | | (m, 1H), 2.67-2.53 (m, 3H), 2.10 (s, 3H), 1.91-1.75 (m, |
| | | | 2H), 1.66-1.47 (m, 3H), 1.30-0.99 (m, 2H). |
| 32 | 11.2 | Me₂NCOCl G | 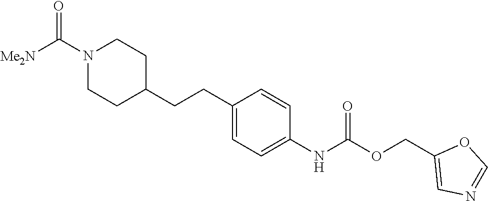 [SMILES: CN(C)C(=O)N1CCC(CCc2ccc(NC(=O)OCc3cnco3)cc2)CC1] |
| | | | Compound 32: oxazol-5-ylmethyl (4-(2-(1- |
| | | | (dimethylcarbamoyl)piperidin-4- |
| | | | yl)ethyl)phenyl)carbamate LRMS (ES) m/z: 372 |
| | | | [M + H]⁺. ¹H NMR (400 MHz, Methanol-d₄) δ 8.24 (s, |
| | | | 1H), 7.34 (d, J = 8.1 Hz, 2H), 7.25 (s, 1H), 7.18-7.02 (m, |
| | | | 2H), 5.34-5.12 (m, 2H), 3.66 (dt, J = 13.0, 2.5 Hz, 2H), |
| | | | 2.83 (s, 6H), 2.75 (td, J = 12.8, 2.5 Hz, 2H), 2.67-2.51 |
| | | | (m, 2H), 1.83-1.69 (m, 2H), 1.69-1.52 (m, 2H), 1.52- |
| | | | 1.40 (m, 1H), 1.31-1.09 (m, 2H). |
| 120 | 11.2 | MeOCOCl D | 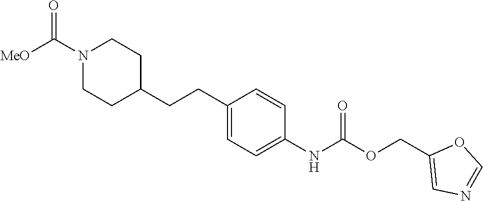 [SMILES: COC(=O)N1CCC(CCc2ccc(NC(=O)OCc3cnco3)cc2)CC1] |
| | | | Compound 120: methyl 4-(4-(((oxazol-5- |
| | | | ylmethoxy)carbonyl)amino)phenethyl)piperidine-1- |
| | | | carboxylate LRMS (ES) m/z: 388 [M + H]⁺. ¹H NMR (400 |
| | | | MHz, Methanol-d₄) δ 8.24 (s, 1H), 7.34 (d, J = 8.1 Hz, 2H), |
| | | | 7.25 (s, 1H), 7.20-6.98 (m, 2H), 5.23 (s, 2H), 4.10 (d, J = |
| | | | 13.3 Hz, 2H), 3.68 (s, 3H), 2.86-2.68 (m, 2H), 2.62 (dd, |
| | | | J = 9.0, 6.6 Hz, 2H), 1.84-1.70 (m, 2H), 1.64-1.38 (m, |
| | | | 3H), 1.20-1.05 (m, 2H). |
| 132 | 11.2 | oxetan-3-one A | 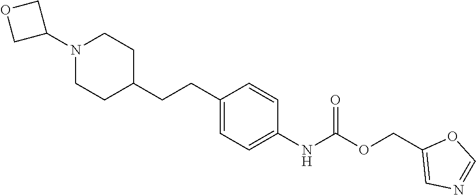 [SMILES: O=C(Nc1ccc(CCC2CCN(C3COC3)CC2)cc1)OCc1cnco1] |
| | | | Compound 132: oxazol-5-ylmethyl (4-(2-(1-(oxetan-3- |
| | | | yl)piperidin-4-yl)ethyl)phenyl)carbamate: LRMS (ES) |
| | | | m/z: 386 [M + H]⁺. ¹H NMR (400 MHz, Methanol-d₄) δ 8.24 |
| | | | (s, 1H), 7.34 (d, J = 8.2 Hz, 2H), 7.25 (s, 1H), 7.20-7.03 |
| | | | (m, 2H), 5.25 (s, 2H), 4.73 (t, J = 7.0 Hz, 2H), 4.66 (t, J = |
| | | | 6.5 Hz, 2H), 3.80-3.63 (m, 1H), 2.97 (d, J = 11.7 Hz, 2H), |
| | | | 2.67-2.54 (m, 2H), 2.19-2.04 (m, 2H), 1.92-1.78 (m, |
| | | | 2H), 1.66-1.52 (m, 2H), 1.46-1.30 (m, 3H). |
| 223 | 25.2 | oxetan-3-one A | 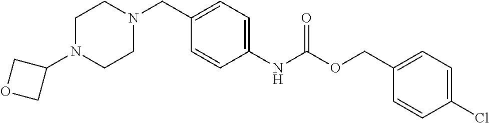 [SMILES: O=C(Nc1ccc(CN2CCN(C3COC3)CC2)cc1)OCc1ccc(Cl)cc1] |
| | | | Compound 223: (4-chlorophenyl)methyl N-(4-{[4- |
| | | | (oxetan-3-yl)piperazin-1-yl]methyl}phenyl)carbamate. |
| | | | LCMS-ESI (POS.) m/z: 416.0 [M + H]+. ¹H NMR (400 |
| | | | MHz, DMSO-d6) δ 9.76 (s, 1H), 7.42 (d, J = 28.2 Hz, 6H), |
| | | | 7.18 (d, J = 8.4 Hz, 2H), 5.13 (s, 2H), 4.45 (dt, J = 43.6, 6.3 |
| | | | Hz, 4H), 3.36 (d, J = 14.6 Hz, 4H), 2.24 (s, 7H). |
| 318 | 26.2 | oxetan-3-one A | 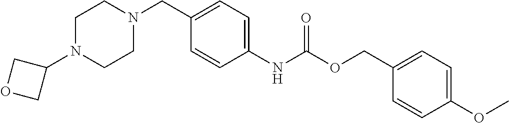 [SMILES: COc1ccc(COC(=O)Nc2ccc(CN3CCN(C4COC4)CC3)cc2)cc1] |
| | | | Compound 318: (4-methoxyphenyl)methyl N-(4-{[4- |
| | | | (oxetan-3-yl)piperazin-1-yl]methyl}phenyl)carbamate. |
| | | | LCMS-ESI (POS.) m/z: 412.0 [M + H]⁺. ¹H NMR (300 |
| | | | MHz, DMSO-d6) δ 9.66 (s, 1H), 7.37 (t, J = 8.4 Hz, 4H), |
| | | | 7.17 (d, J = 8.4 Hz, 2H), 6.94 (d, J = 8.6 Hz, 2H), 5.06 (s, |
| | | | 2H), 4.45 (dt, J = 32.7, 6.3 Hz, 4H), 3.75 (s, 3H), 2.33 (d, |
| | | | J = 10.8 Hz, 9H). |
| 29 | 25.2 | 2- chloropyridine I | 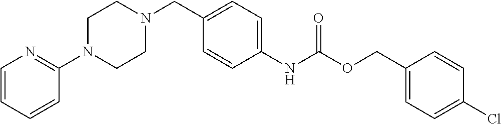 [SMILES: O=C(Nc1ccc(CN2CCN(c3ccccn3)CC2)cc1)OCc1ccc(Cl)cc1] |
| | | | Compound 29: (4-chlorophenyl)methyl N-(4-{[4- |
| | | | (pyridin-2-yl)piperazin-1-yl]methyl}phenyl)carbamate. |
| | | | LCMS-ESI (POS.) m/z: 437.0 [M + H]⁺. ¹H NMR (300 |
| | | | MHz, DMSO-d6) δ 9.77 (s, 1H), 8.09 (dd, J = 5.0, 1.9 Hz, |
| | | | 1H), 7.54-7.38 (m, 7H), 7.23 (d, J = 8.3 Hz, 2H), 6.79 (d, |
| | | | J = 8.6 Hz, 1H), 6.62 (dd, J = 7.0, 4.9 Hz, 1H), 5.14 (s, 2H), |
| | | | 3.44 (d, J = 5.2 Hz, 6H), 2.42 (t, J = 5.1 Hz, 4H). |
| 83 | 26.2 | 2- chloropyridine | 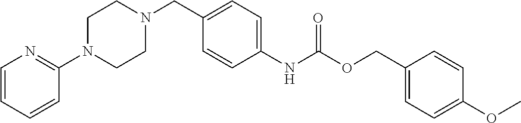 [SMILES: COc1ccc(COC(=O)Nc2ccc(CN3CCN(c4ccccn4)CC3)cc2)cc1] |
| | | | Compound 83: (4-methoxyphenyl)methyl N-(4-{[4- |
| | | | (pyridin-2-yl)piperazin-1-yl]methyl}phenyl)carbamate. |
| | | | LCMS-ESI (POS.) m/z: 433.0 [M + H]⁺. ¹H NMR (400 |
| | | | MHz, DMSO-d6) δ 9.69 (s, 1H), 8.09 (dd, J = 5.1, 1.9 Hz, |
| | | | 1H), 7.55-7.46 (m, 1H), 7.41 (d, J = 8.0 Hz, 2H), 7.37 (d, |
| | | | J = 8.2 Hz, 2H), 7.22 (d, J = 8.1 Hz, 2H), 6.95 (d, J = 8.2 |
| | | | Hz, 2H), 6.79 (d, J = 8.6 Hz, 1H), 6.62 (dd, J = 7.2, 4.9 Hz, |
| | | | 1H), 5.07 (s, 2H), 3.75 (s, 3H), 3.48-3.40 (m, 6H), 2.42 (d, |
| | | | J = 5.1 Hz, 4H). |
| 73 | 25.2 | 2-chloro- pyrimidine I | 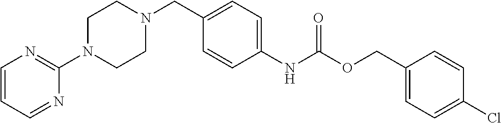 [SMILES: O=C(Nc1ccc(CN2CCN(c3ncccn3)CC2)cc1)OCc1ccc(Cl)cc1] |
| | | | Compound 73: 4-chlorobenzyl (4-((4-(pyrimidin-2- |
| | | | yl)piperazin-1-yl) |
| | | | methyl)phenyl)carbamate. LCMS-ES (Pos) m/z: 438 |
| | | | [M + H]⁺. 1H NMR (400 MHz, DMSO-d6) δ 9.76 (s, 1H), |
| | | | 8.33 (d, J = 4.7 Hz, 2H), 7.49-7.44 (m, 4H), 7.42 (d, J = |
| | | | 8.2 Hz, 2H), 7.22 (d, J = 8.5 Hz, 2H), 6.60 (t, J = 4.7 Hz, |
| | | | 1H), 5.14 (s, 2H), 3.73-3.67 (m, 4H), 3.43 (s, 2H), 2.41- |
| | | | 2.35 (m, 4H). |
| 107 | 25.2 | MeSO₂Cl E | 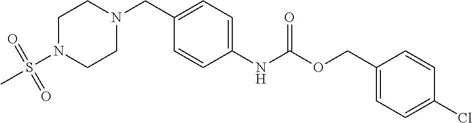 [SMILES: CS(=O)(=O)N1CCN(Cc2ccc(NC(=O)OCc3ccc(Cl)cc3)cc2)CC1] |
| | | | Compound 107: (4-chlorophenyl)methyl N-{4-[(4- |
| | | | methanesulfonylpiperazin-1- |
| | | | yl)methyl]phenyl}carbamate |
| | | | LCMS-ES (Pos) m/z: 438 [M + H]⁺. ¹H NMR (400 MHz, |
| | | | DMSO-d₆) δ 9.78 (s, 1H), 7.50-7.37 (m, 6H), 7.29-7.15 |
| | | | (m, 2H), 5.14 (s, 2H), 3.51-3.39 (m, 2H), 3.14-3.04 (m, |
| | | | 4H), 2.95-2.81 (m, 4H), 2.42 (bs, 3H). |
| 177 | 25.2 | 2,2- dimethyl- oxirane I | 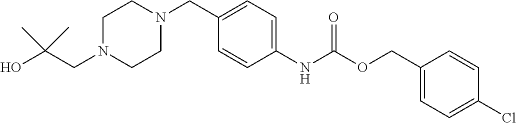 [SMILES: CC(C)(O)CN1CCN(Cc2ccc(NC(=O)OCc3ccc(Cl)cc3)cc2)CC1] |
| | | | Compound 177: 4-chlorobenzyl (4-((4-(2-hydroxy-2- |
| | | | methylpropyl)piperazin-1-yl)methyl)phenyl)carbamate |
| | | | LCMS-ES (Pos) m/z: 432 [M + H]⁺. ¹H NMR (400 MHz, |
| | | | DMSO-d₆) δ 9.75 (s, 1H), 8.15 (s, 1H), 7.49-7.42 (m, 4H), |
| | | | 7.42-7.35 (m, 2H), 7.22-7.15 (m, 2H), 5.13 (s, 2H), 3.42 |
| | | | (s, 1H), 3.38 (s, 2H), 3.34 (dd, J = 10.0, 5.1 Hz, 1H), 2.57- |
| | | | 2.48 (m, 3H), 2.42-2.23 (m, 4H), 2.18 (s, 1H), 1.06 (s, |
| | | | 6H). |
| 191 | 25.2 | Ac₂O B | 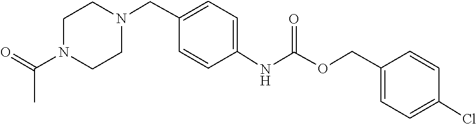 [SMILES: CC(=O)N1CCN(Cc2ccc(NC(=O)OCc3ccc(Cl)cc3)cc2)CC1] |
| | | | Compound 191: (4-chlorophenyl)methyl N-{4-[(4- |
| | | | acetylpiperazin-1-yl)methyl]phenyl}carbamate. |
| | | | LCMS-ES (Pos) m/z: 402 [M + H]⁺. ¹H NMR (400 MHz, |
| | | | DMSO-d₆) δ 9.85 (s, 1H), 7.55-7.39 (m, 6H), 7.33-7.18 |
| | | | (m, 2H), 5.14 (s, 2H), 3.86-3.09 (m, 9H), 2.45-2.22 (m, |
| | | | 1H), 1.98 (s, 3H). |
| 269 | oxazol- 5- ylmethyl (4-(2- (piperazin- 1- yl)ethyl) phenyl) carbamate | oxetan-3-one A | 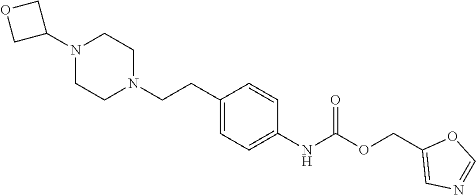 [SMILES: O=C(Nc1ccc(CCN2CCN(C3COC3)CC2)cc1)OCc1cnco1] |
| | | | Compound 269: 1,3-oxazol-5-ylmethyl N-(4-{2-[4- |
| | | | (oxetan-3-yl)piperazin-1-yl]ethyl}phenyl)carbamate. |
| | | | LCMS-ES (Pos) m/z: 387 [M + H]⁺. ¹H NMR (400 MHz, |
| | | | DMSO-d₆) δ 9.71 (s, 1H), 8.41 (s, 1H), 7.34 (d, J = 8.0 |
| | | | Hz, 2H), 7.30 (s, 1H), 7.12 (d, J = 8.0 Hz, 2H), 5.20 (s, |
| | | | 2H), 4.51 (t, J = 6.5 Hz, 2H), 4.41 (t, J = 6.1 Hz, 2H), 3.44- |
| | | | 3.34 (m, 3H), 2.69-2.62 (m, 2H), 2.55-2.40 (m, 4H), |
| | | | 2.35-2.15 (m, 4H). |
| 232 | 13.2 | oxetan-3-one A | 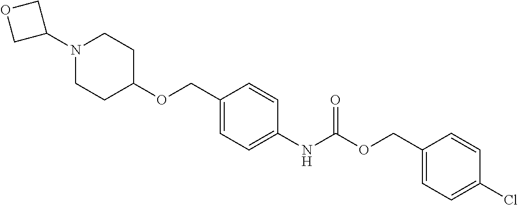 [SMILES: O=C(Nc1ccc(COC2CCN(C3COC3)CC2)cc1)OCc1ccc(Cl)cc1] |
| | | | Compound 232: 4-chlorobenzyl (4-(((1-(oxetan-3- |
| | | | yl)piperidin-4-yl)oxy)methyl)phenyl)carbamate. |
| | | | LCMS-APCI (POS.) m/z: 431.1 [M + H]⁺. 1H NMR (400 |
| | | | MHz, Methanol-d4) δ 8.44 (s, 1H), 7.30 (p, J = 8.1 Hz, 6H), |
| | | | 7.18 (t, J = 9.3 Hz, 2H), 5.06 (s, 2H), 4.57 (t, J = 6.8 Hz, |
| | | | 1H), 4.49 (d, J = 6.5 Hz, 1H), 4.43 (s, 1H), 4.39 (s, 1H), |
| | | | 3.66 (s, 1H), 2.99 (dd, J = 12.3, 5.9 Hz, 2H), 2.52 (s, 1H), |
| | | | 2.03-1.74 (m, 5H), 1.58 (s, 1H), 1.19 (s, 1H). |
| 93 | 13.2 | Ac₂O B | 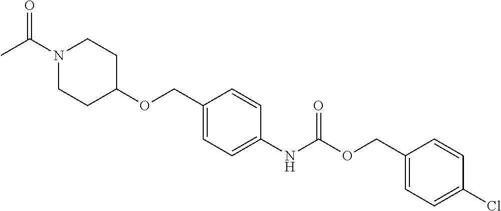 [SMILES: CC(=O)N1CCC(OCc2ccc(NC(=O)OCc3ccc(Cl)cc3)cc2)CC1] |
| | | | Compound 93: 4-chlorobenzyl (4-(((1-acetylpiperidin-4- |
| | | | yl)oxy)methyl)phenyl)carbamate. |
| | | | (LCMS-APCI (POS.) m/z: 417.1 [M + H]⁺. ¹H NMR (400 |
| | | | MHz, Methanol-d₄) δ 7.44-7.35 (m, 6H), 7.28 (d, J = 8.2 |
| | | | Hz, 2H), 5.16 (s, 2H), 4.52 (s, 2H), 3.93-3.85 (m, 1H), |
| | | | 3.77-3.64 (m, 2H), 2.09 (s, 3H), 2.07-1.99 (m, 2H), 1.97- |
| | | | 1.83 (m, 2H), 1.66-1.50 (m, 2H). |
| 354 | 14.2 | oxetan-3-one A | 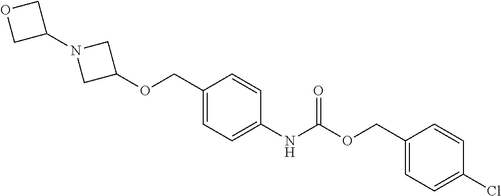 [SMILES: O=C(Nc1ccc(COC2CN(C3COC3)C2)cc1)OCc1ccc(Cl)cc1] |
| | | | Compound 354: 4-chlorobenzyl (4-(((1-(oxetan-3- |
| | | | yl)azetidin-3-yl)oxy)methyl)phenyl)carbamate. |
| | | | (LCMS-APCI (POS.) m/z: 403.1 [M + H]⁺. 1H NMR (400 |
| | | | MHz, Methanol-d4) δ 7.36-7.25 (m, 6H), 7.17 (d, J = 8.5 |
| | | | Hz, 2H), 5.06 (s, 2H), 4.62 (t, J = 6.8 Hz, 2H), 4.35 (dd, J = |
| | | | 7.0, 4.9 Hz, 2H), 4.32 (s, 2H), 4.17 (p, J = 5.9 Hz, 1H), 3.73 |
| | | | (ddd, J = 11.5, 6.6, 5.0 Hz, 1H), 3.53 (td, J = 6.5, 1.9 Hz, |
| | | | 2H), 3.09-3.03 (m, 2H). |
| 16 | 14.2 | Ac₂O B | 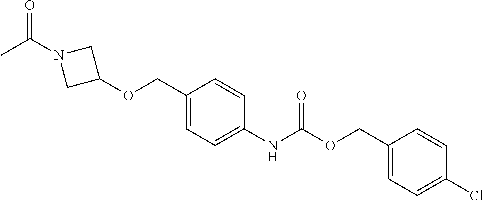 [SMILES: CC(=O)N1CC(OCc2ccc(NC(=O)OCc3ccc(Cl)cc3)cc2)C1] |
| | | | Compound 16: 4-chlorobenzyl (4-(((1-acetylazetidin-3- |
| | | | yl)oxy)methyl)phenyl)carbamate. |
| | | | LCMS-APCI (POS.) m/z: 389.1 [M + H]⁺. ¹H NMR (400 |
| | | | MHz, Methanol-d₄) δ 7.47-7.34 (m, 6H), 7.31-7.25 (m, |
| | | | 2H), 5.16 (s, 2H), 4.59-4.52 (m, 1H), 4.45 (s, 1H), 4.42- |
| | | | 4.36 (m, 1H), 4.35-4.28 (m, 1H), 4.10 (dd, J = 10.7, 6.6 |
| | | | Hz, 1H), 4.02 (dd, J = 9.6, 3.8 Hz, 1H), 3.77 (dd, J = 10.9, |
| | | | 4.0 Hz, 1H), 1.85 (s, 3H). |
| 357 | 31.1 | isobutyryl chloride | 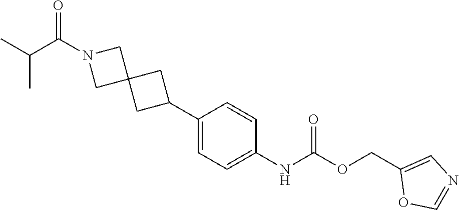 [SMILES: CC(C)C(=O)N1CC2(CC(c3ccc(NC(=O)OCc4cnco4)cc3)C2)C1] |
| | | | Compound 357: oxazol-5-ylmethyl (4-(2-isobutyryl-2- |
| | | | azaspiro[3.3]heptan-6-yl)phenyl)carbamate |
| | | | LCMS-ESI (POS.) m/z: 384.1 [M + H]⁺. |
| | | | ¹H NMR (400 MHz, DMSO-d₆) δ 9.73 (s, 1H), 8.41 (s, |
| | | | 1H), 7.36 (d, J = 8.2 Hz, 2H), 7.30 (s, 1H), 7.13 (d, J = 8.4 |
| | | | Hz, 2H), 5.20 (s, 2H), 4.26 (s, 1H), 4.05 (s, 1H), 3.94 (s, |
| | | | 1H), 3.73 (s, 1H), 2.67 (s, 1H), 2.25-2.16 (m, 3H), 1.72 |
| | | | (s, 1H), 1.50 (s, 1H), 0.95 (t, J = 6.7 Hz, 6H). |
| 358 | 31.1 | acetic anhydride | 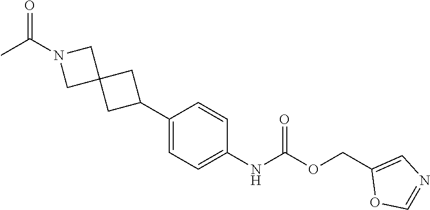 [SMILES: CC(=O)N1CC2(CC(c3ccc(NC(=O)OCc4cnco4)cc3)C2)C1] |
| | | | Compound 358: oxazol-5-ylmethyl (4-(2-acetyl-2- |
| | | | azaspiro[3.3]heptan-6-yl)phenyl)carbamate |
| | | | LCMS-ESI (POS.) m/z: 356.1 [M + H]⁺. |
| | | | ¹H NMR (400 MHz, DMSO-d₆) δ 9.70 (s, 1H), 8.39 (s, |
| | | | 1H), 7.34 (d, J = 8.2 Hz, 2H), 7.27 (s, 1H), 7.11 (d, J = 8.6 |
| | | | Hz, 2H), 5.18 (s, 3H), 4.20 (s, 1H), 3.98 (s, 1H), 3.91 (s, |
| | | | 1H), 3.70 (s, 1H), 2.18 (ddd, J = 12.0, 9.5, 2.4 Hz, 3H), |
| | | | 1.71 (d, J = 10.3 Hz, 4H). |
| 359 | 30.1 | methane- sulfonyl chloride | 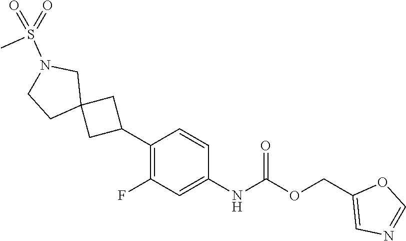 [SMILES: CS(=O)(=O)N1CCC2(CC(c3ccc(NC(=O)OCc4cnco4)cc3F)C2)C1] |
| | | | Compound 359 |
| | | | diastereoisomer 1 obtained after separation |
| | | | LCMS-ESI (POS.) m/z: 424.1 [M + H]⁺. |
| | | | ¹H NMR (400 MHz, DMSO-d₆) δ 9.96 (s, 1H), 8.42 (s, |
| | | | 1H), 7.31 (d, J = 1.8 Hz, 1H), 7.30-7.24 (m, 2H), 7.18 |
| | | | (dd, J = 8.5, 2.1 Hz, 1H), 5.22 (s, 2H), 3.59 (p, J = 9.1 Hz, |
| | | | 1H), 3.29 (t, J = 6.8 Hz, 2H), 3.17 (s, 2H), 2.86 (s, 3H), |
| | | | 2.26 (td, J = 8.9, 2.0, 2H), 2.17 (td, J = 9.6, 2.5 Hz, 2H), |
| | | | 2.06 (t, J = 6.7 Hz, 2H). |
| 360 | 30.1 | methane- sulfonyl chloride | 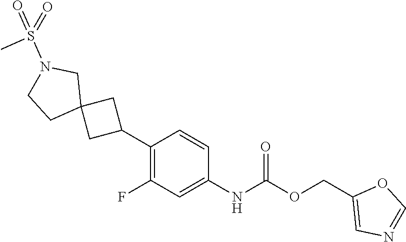 [SMILES: CS(=O)(=O)N1CCC2(CC(c3ccc(NC(=O)OCc4cnco4)cc3F)C2)C1] |
| | | | Compound 360 |
| | | | diastereoisomer 2 obtained after separation |
| | | | LCMS-ESI (POS.) m/z: 424.1 [M + H]⁺. |
| | | | ¹H NMR (400 MHz, DMSO-d₆) δ 9.96 (s, 1H), 8.42 (s, |
| | | | 1H), 7.31 (s, 1H), 7.30-7.23 (m, 2H), 7.18 (dd, J = 8.3, |
| | | | 2.1 Hz, 1H), 5.22 (s, 2H), 3.61 (p, J = 9.1 Hz, 1H), 3.39 (s, |
| | | | 2H), 3.21 (t, J = 6.9 Hz, 2H), 2.91 (s, 3H), 2.36 (td, J = |
| | | | 8.9, 2.4 Hz, 2H), 2.11 (td, J = 9.6, 2.7 Hz, 2H), 1.87 (t, J = |
| | | | 7.0 Hz, 2H). |
| 361 | 30.1 | isobutyryl chloride | 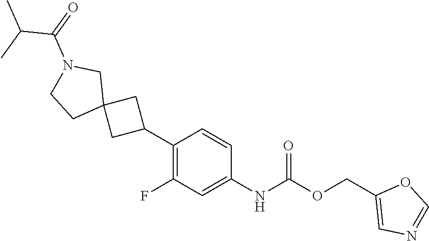 [SMILES: CC(C)C(=O)N1CCC2(CC(c3ccc(NC(=O)OCc4cnco4)cc3F)C2)C1] |
| | | | Compound 361 |
| | | | diastereoisomer 1 obtained after separation |
| | | | LCMS-ESI (POS.) m/z: 416.1 [M + H]⁺. |
| | | | ¹H NMR (400 MHz, DMSO-d₆) δ 9.96 (s, 1H), 8.42 (s, |
| | | | 1H), 7.31 (s, 1H), 7.30-7.24 (m, 2H), 7.21-7.14 (m, |
| | | | 1H), 5.22 (s, 2H), 3.67-3.56 (m, 1H), 3.53 (t, J = 6.8 Hz, |
| | | | 1H), 3.41 (s, 1H), 3.35-3.30 (m, 1H), 3.22 (s, 1H), 2.68- |
| | | | 2.54 (m, 1H), 2.25 (dd, J = 8.6, 2.1 Hz, 2H), 2.20-2.10 |
| | | | (m, 2H), 2.08 (t, J = 6.9 Hz, 1H), 1.98 (t, J = 6.9 Hz, 1H), |
| | | | 0.98 (d, J = 2.3 Hz, 3H), 0.97 (d, J = 2.3 Hz, 3H). |
| 362 | 30.1 | isobutyryl chloride | 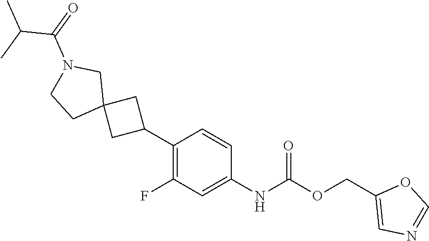 [SMILES: CC(C)C(=O)N1CCC2(CC(c3ccc(NC(=O)OCc4cnco4)cc3F)C2)C1] |
| | | | Compound 362 |
| | | | diastereoisomer 2 obtained after separation |
| | | | LCMS-ESI (POS.) m/z: 416.1 [M + H]⁺. |
| | | | ¹H NMR (400 MHz, DMSO-d₆) δ 9.96 (s, 1H), 8.42 (s, |
| | | | 1H), 7.31 (s, 1H), 7.30-7.23 (m, 2H), 7.18 (dt, J = 8.4, |
| | | | 1.7 Hz, 1H), 5.22 (s, 2H), 3.70-3.54 (m, 2H), 3.48-3.41 |
| | | | (m, 2H), 3.25 (t, J = 7.1 Hz, 1H), 2.74-2.61 (m, 1H), |
| | | | 2.34-2.25 (m, 2H), 2.13-2.06 (m, 2H), 1.88 (t, J = 7.0 |
| | | | Hz, 1H), 1.78 (t, J = 7.1 Hz, 1H), 1.01 (d, J = 6.7 Hz, 3H), |
| | | | 0.99 (d, J = 6.7 Hz, 3H). |
| 363 | 30.1 | dimethyl- carbamyl chloride | 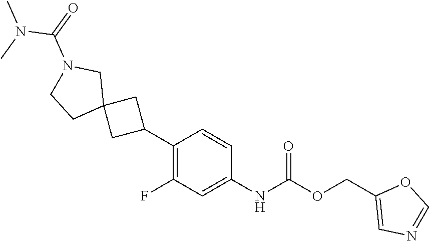 [SMILES: CN(C)C(=O)N1CCC2(CC(c3ccc(NC(=O)OCc4cnco4)cc3F)C2)C1] |
| | | | Compound 363 |
| | | | diastereoisomer 1 obtained after separation |
| | | | LCMS-ESI (POS.) m/z: 417.1 [M + H]⁺. ¹H NMR (400 |
| | | | MHz, DMSO-d₆) δ 9.96 (s, 1H), 8.42 (s, 1H), 7.31 (s, 1H), |
| | | | 7.30-7.24 (m, 2H), 7.17 (dd, J = 8.5, 2.1 Hz, 1H), 5.22 |
| | | | (s, 2H), 3.58 (p, J = 9.1 Hz, 1H), 3.33-3.28 (m, 2H), 3.19 |
| | | | (s, 2H), 2.71 (s, 6H), 2.23 (td, J = 8.7, 2.7 Hz, 2H), 2.09 |
| | | | (td, J = 9.5, 2.6 Hz, 2H), 1.94 (t, J = 6.7 Hz, 2H). |
| 364 | 30.1 | dimethyl- carbamyl chloride | 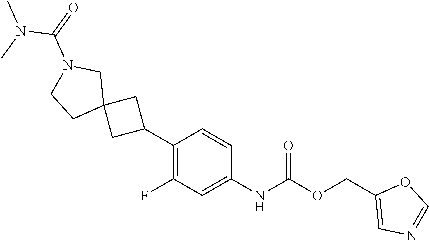 [SMILES: CN(C)C(=O)N1CCC2(CC(c3ccc(NC(=O)OCc4cnco4)cc3F)C2)C1] |
| | | | Compound 364 |
| | | | diastereoisomer 2 obtained after separation |
| | | | LCMS-ESI (POS.) m/z: 417.1 [M + H]⁺. ¹H NMR (400 |
| | | | MHz, DMSO-d₆) δ 9.96 (s, 1H), 8.42 (s, 1H), 7.31 (s, 1H), |
| | | | 7.30-7.21 (m, 2H), 7.17 (dd, J = 8.5, 2.1 Hz, 1H), 5.22 |
| | | | (s, 2H), 3.59 (p, J = 9.1 Hz, 1H), 3.41 (s, 1H), 3.33-3.29 |
| | | | (m, 1H), 3.24 (t, J = 6.9 Hz, 2H), 2.74 (s, 6H), 2.26 |
| | | | (td, J = 9.0, 2.4 Hz, 2H), 2.07 (td, J = 9.6, 2.7 Hz, 2H), |
| | | | 1.75 (t, J = 6.9 Hz, 2H). |
| 372 | 30.1 | acetic anhydride | 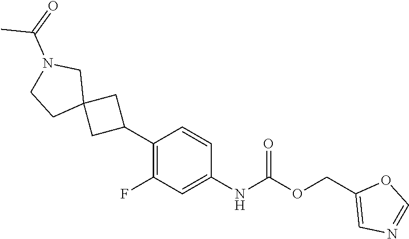 [SMILES: CC(=O)N1CCC2(CC(c3ccc(NC(=O)OCc4cnco4)cc3F)C2)C1] |
| | | | Compound 372 |
| | | | diastereoisomer 1 obtained after separation |
| | | | LCMS-ESI (POS.) m/z: 388.1 [M + H]⁺. ¹H NMR (400 |
| | | | MHz, DMSO-d₆) δ 9.96 (s, 1H), 8.42 (s, 1H), 7.31 (s, 1H), |
| | | | 7.30-7.24 (m, 2H), 7.18 (ddd, J = 8.2, 5.1, 2.0 Hz, 1H), |
| | | | 5.22 (s, 2H), 3.65-3.54 (m, 1H), 3.47 (t, J = 6.8 Hz, 1H), |
| | | | 3.36 (s, 1H), 3.34-3.29 (m, 1H), 3.21 (s, 1H), 2.25 |
| | | | (td, J = 10.0, 8.8, 2.1 Hz, 2H), 2.19-2.03 (m, 3H), 1.98 |
| | | | (t, J = 7.0 Hz, 1H), 1.92-1.87 (m, 3H). |
| 373 | 30.1 | acetic anhydride | 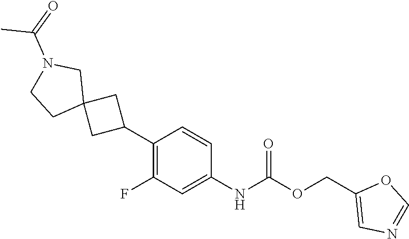 [SMILES: CC(=O)N1CCC2(CC(c3ccc(NC(=O)OCc4cnco4)cc3F)C2)C1] |
| | | | Compound 373 |
| | | | diastereoisomer 2 obtained after separation |
| | | | LCMS-ESI (POS.) m/z: 388.1 [M + H]⁺. ¹H NMR (400 |
| | | | MHz, DMSO-d₆) δ 9.97 (s, 1H), 8.43 (s, 1H), 7.32 (s, 1H), |
| | | | 7.31-7.24 (m, 2H), 7.19 (dt, J = 8.6, 2.0 Hz, 1H), 5.23 (s, |
| | | | 2H), 3.68-3.54 (m, 2H), 3.45 (s, 1H), 3.40 (t, J = 7.0 Hz, |
| | | | 1H), 3.25 (t, J = 7.1 Hz, 1H), 2.35-2.22 (m, 2H), 2.16- |
| | | | 2.07 (m, 2H), 1.98-1.92 (m, 3H), 1.88 (t, J = 7.0 Hz, |
| | | | 1H), 1.79 (t, J = 7.1 Hz, 1H). |
| 375 | 30.2 | acetic anhydride | 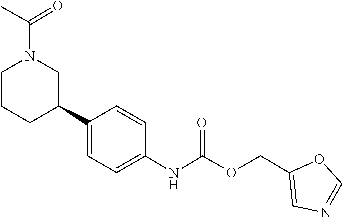 [SMILES: CC(=O)N1CCC[C@H](c2ccc(NC(=O)OCc3cnco3)cc2)C1] |
| | | | Compound 375: oxazol-5-ylmethyl (R)-(4-(1- |
| | | | acetylpiperidin-3-yl)phenyl)carbamate LCMS-ESI |
| | | | (POS.) m/z: 344.1 [M + H]⁺. ¹H NMR (400 MHz, DMSO- |
| | | | d₆) δ 9.76 (s, 1H), 8.43 (s, 1H), 7.44-7.34 (m, 2H), 7.31 |
| | | | (s, 1H), 7.26-7.13 (m, 2H), 5.22 (s, 2H), 4.41 (dd, J = |
| | | | 20.2, 12.0 Hz, 1H), 3.79 (dd, J = 30.9, 14.0 Hz, 1H), 3.18 |
| | | | (d, J = 5.2 Hz, 1H), 3.11-2.99 (m, 1H), 2.02 (d, J = 5.6 |
| | | | Hz, 3H), 1.88 (d, J = 12.4 Hz, 1H), 1.79-1.58 (m, 2H), |
| | | | 1.57-1.29 (m, 2H). |
| 365 | 30.2 | methane- sulfonyl chloride | 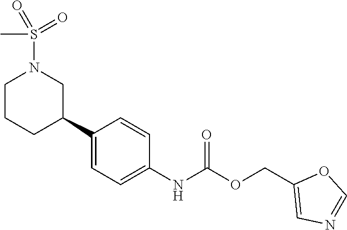 [SMILES: CS(=O)(=O)N1CCC[C@H](c2ccc(NC(=O)OCc3cnco3)cc2)C1] |
| | | | Compound 365: oxazol-5-ylmethyl (R)-(4-(1- |
| | | | (methylsulfonyl)piperidin-3-yl)phenyl)carbamate |
| | | | LCMS-ESI (POS.) m/z: 380.1 [M + H]⁺ |
| | | | ¹H NMR (400 MHz, DMSO-d₆) δ 9.76 (s, 1H), 8.42 (s, |
| | | | 1H), 7.39 (d, J = 8.3 Hz, 2H), 7.30 (s, 1H), 7.26-7.18 (m, |
| | | | 2H), 5.21 (s, 2H), 3.55 (dd, J = 22.8, 9.7 Hz, 2H), 2.87 (s, |
| | | | 3H), 2.78-2.65 (m, 3H), 1.89-1.78 (m, 2H), 1.68-1.49 |
| | | | (m, 2H). |
| 366 | 30.2 | dimethyl- sulfamoyl chloride | 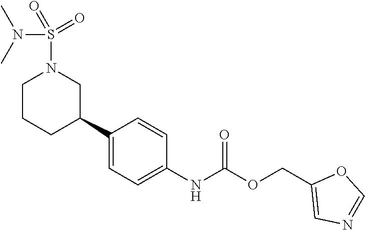 [SMILES: CN(C)S(=O)(=O)N1CCC[C@H](c2ccc(NC(=O)OCc3cnco3)cc2)C1] |
| | | | Compound 366: oxazol-5-ylmethyl (R)-(4-(1-(N,N- |
| | | | dimethylsulfamoyl)piperidin-3-yl)phenyl)carbamate. |
| | | | LCMS-ESI (POS.) m/z: 409.1 [M + H]⁺. |
| | | | ¹H NMR (400 MHz, DMSO-d₆) δ 9.77 (s, 1H), 8.43 (s, |
| | | | 1H), 7.39 (d, J = 8.2 Hz, 2H), 7.31 (s, 1H), 7.21 (d, J = 8.6 |
| | | | Hz, 2H), 5.22 (s, 2H), 3.66-3.47 (m, 2H), 2.93-2.81 (m, |
| | | | 2H), 2.75 (s, 6H), 2.68 (s, 1H), 1.91-1.73 (m, 2H), 1.63- |
| | | | 1.51 (m, 2H). |
| 368 | 378 | isobutyryl chloride | 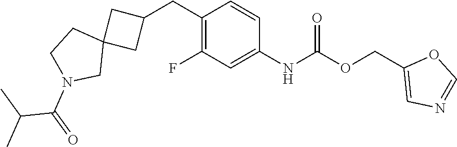 [SMILES: CC(C)C(=O)N1CCC2(CC(Cc3ccc(NC(=O)OCc4cnco4)cc3F)C2)C1] |
| | | | Compound 368: oxazol-5-ylmethyl (3-fluoro-4-((6- |
| | | | isobutyryl-6-azaspiro[3.4]octan-2- |
| | | | yl)methyl)phenyl)carbamate. |
| | | | LCMS-ESI (POS.) m/z: 430.1 [M + H]⁺. |
| | | | ¹H NMR (400 MHz, DMSO-d₆) δ 9.95 (s, 1H), 8.42 (s, |
| | | | 1H), 7.34-7.27 (m, 2H), 7.20-7.10 (m, 2H), 5.23 (s, |
| | | | 2H), 3.50-3.38 (m, 2H), 3.30-3.16 (m, 2H), 2.70-2.54 |
| | | | (m, 3H), 2.04-1.88 (m, 3H), 1.86-1.63 (m, 4H), 0.97 |
| | | | (dd, J = 6.7, 1.8 Hz, 6H). |
| 379 | 378 | dimethyl- carbamoyl chloride | 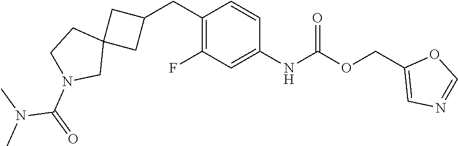 [SMILES: CN(C)C(=O)N1CCC2(CC(Cc3ccc(NC(=O)OCc4cnco4)cc3F)C2)C1] |
| | | | Compound 379: oxazol-5-ylmethyl (4-((6- |
| | | | (dimethylcarbamoyl)-6-azaspiro[3.4]octan-2- |
| | | | yl)methyl)-3-fluorophenyl)carbamate |
| | | | LCMS-ESI (POS.) m/z: 431.1 [M + H]⁺. |
| | | | ¹H NMR (400 MHz, DMSO-d₆) δ 9.94 (s, 1H), 8.42 (s, |
| | | | 1H), 7.35-7.23 (m, 2H), 7.21-7.06 (m, 2H), 5.22 (s, |
| | | | 2H), 3.25-3.17 (m, 3H), 3.12 (s, 1H), 2.69 (s, 6H), 2.66- |
| | | | 2.59 (m, 2H), 2.47-2.39 (m, 1H), 1.97-1.86 (m, 2H), |
| | | | 1.76 (t, J = 6.7 Hz, 1H), 1.73-1.60 (m, 3H). |
| 374 | 378 | acetic anhydride | 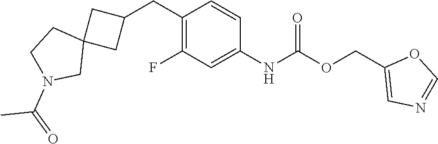 [SMILES: CC(=O)N1CCC2(CC(Cc3ccc(NC(=O)OCc4cnco4)cc3F)C2)C1] |
| | | | Compound 374: oxazol-5-ylmethyl (4-((6-acetyl-6- |
| | | | azaspiro[3.4]octan-2-yl)methyl)-3- |
| | | | fluorophenyl)carbamate |
| | | | LCMS-ESI (POS.) m/z: 402.1 [M + H]⁺ |
| | | | ¹H NMR (400 MHz, DMSO-d₆) δ 9.95 (s, 1H), 8.42 (s, |
| | | | 1H), 7.33-7.27 (m, 2H), 7.18-7.10 (m, 2H), 5.22 (s, |
| | | | 2H), 3.42-3.35 (m, 2H), 3.29 (s, 1H), 3.27-3.18 (m, |
| | | | 2H), 3.16 (s, 1H), 2.68-2.61 (m, 3H), 1.91-1.97 (m, |
| | | | 4H), 1.85-1.77 (m, 1H), 1.75-1.67 (m, 2H). |
| 371 | 378 | methyl carbono- chloridate | 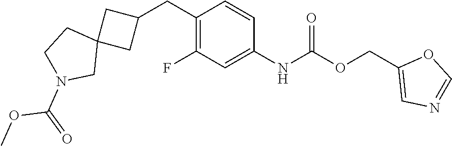 [SMILES: COC(=O)N1CCC2(CC(Cc3ccc(NC(=O)OCc4cnco4)cc3F)C2)C1] |
| | | | Compound 371: methyl 2-(2-fluoro-4-(((oxazol-5- |
| | | | ylmethoxy)carbonyl)amino)benzyl)-6- |
| | | | azaspiro[3.4]octane-6-carboxylate |
| | | | LCMS-ESI (POS.) m/z: 418.1 [M + H]⁺. |
| | | | ¹H NMR (400 MHz, DMSO-d₆) δ 9.93 (s, 1H), 8.41 (s, |
| | | | 1H), 7.31-7.25 (m, 2H), 7.17-7.08 (m, 2H), 5.21 (s, |
| | | | 2H), 3.54 (s, 3H), 3.27-3.18 (m, 4H), 3.14 (s, 2H), 1.99- |
| | | | 1.89 (m, 2H), 1.86-1.79 (m, 2H), 1.75-1.64 (m, 3H). |
| 367 | 378 | propionyl chloride | 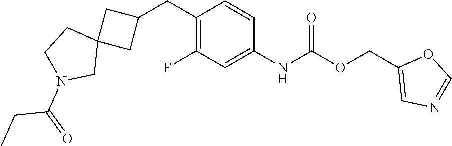 [SMILES: CCC(=O)N1CCC2(CC(Cc3ccc(NC(=O)OCc4cnco4)cc3F)C2)C1] |
| | | | Compound 367: oxazol-5-ylmethyl (3-fluoro-4-((6- |
| | | | propionyl-6-azaspiro[3.4]octan-2- |
| | | | yl)methyl)phenyl)carbamate. |
| | | | LCMS-ESI (POS.) m/z: 416.1 [M + H]⁺. |
| | | | ¹H NMR (400 MHz, DMSO-d₆) δ 9.95 (s, 1H), 8.42 (s, |
| | | | 1H), 7.33-7.27 (m, 2H), 7.20-7.08 (m, 2H), 5.23 (s, |
| | | | 2H), 4.10 (q, J = 5.3 Hz, 2H), 3.18 (d, J = 5.3 Hz, 3H), |
| | | | 2.35-2.31 (m, 1H), 2.18 (t, J = 7.2 Hz, 2H), 2.01-1.88 |
| | | | (m, 3H), 1.86-1.78 (m, 2H), 1.76-1.66 (m, 3H), 0.96 |
| | | | (t, J = 7.4 Hz, 2H). |
| 370 | 378 | methane- sulfonyl chloride | 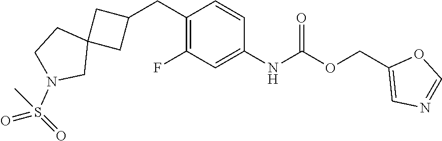 [SMILES: CS(=O)(=O)N1CCC2(CC(Cc3ccc(NC(=O)OCc4cnco4)cc3F)C2)C1] |
| | | | Compound 370: oxazol-5-ylmethyl (3-fluoro-4-((6- |
| | | | (methylsulfonyl)-6-azaspiro[3.4]octan-2- |
| | | | yl)methyl)phenyl)carbamate. |
| | | | LCMS-ESI (POS.) m/z: 438.1 [M + H]⁺. |
| | | | ¹H NMR (400 MHz, DMSO-d₆) δ 9.95 (s, 1H), 8.42 (s, |
| | | | 1H), 7.31 (s, 2H), 7.20-7.07 (m, 2H), 5.22 (s, 2H), 3.24- |
| | | | 3.15 (m, 3H), 3.11 (s, 1H), 2.88-2.81 (m, 3H), 2.68- |
| | | | 2.60 (m,, 2H), 2.09-2.00 (m, 1H), 1.97-1.86 (m, 3H), |
| | | | 1.84-1.65 (m, 3H). |
| 369 | 378 | ethanesulfonyl chloride | 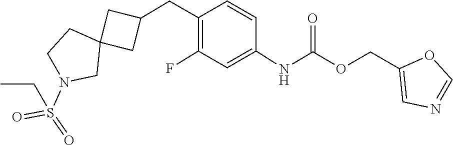 [SMILES: CCS(=O)(=O)N1CCC2(CC(Cc3ccc(NC(=O)OCc4cnco4)cc3F)C2)C1] |
| | | | Compound 369: oxazol-5-ylmethyl (3-fluoro-4-((6- |
| | | | (ethylsulfonyl)-6-azaspiro[3.4]octan-2- |
| | | | yl)methyl)phenyl)carbamate. |
| | | | LCMS-ESI (POS.) m/z: 452.1 [M + H]⁺. |
| | | | ¹H NMR (400 MHz, DMSO-d₆) δ 9.93 (s, 1H), 8.41 (s, |
| | | | 1H), 7.31-7.25 (m, 2H), 7.19-7.07 (m, 2H), 5.21 (s, |
| | | | 2H), 3.25-3.18 (m, 3H), 3.12 (s, 1H), 3.02 (q, J = 7.6 Hz, |
| | | | 3H), 2.05-1.85 (m, 4H), 1.84-1.78 (m 1H), 1.77-1.64 |
| | | | (m, 3H), 1.17 (td, J = 7.3, 1.0 Hz, 3H). |
| 385 | 30.3 | dimethyl- carbamoyl chloride | 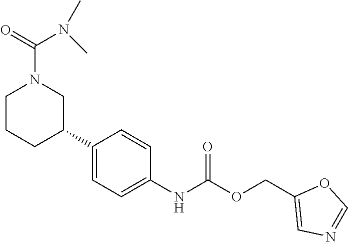 [SMILES: CN(C)C(=O)N1CCC[C@@H](c2ccc(NC(=O)OCc3cnco3)cc2)C1] |
| | | | Compound 385: oxazol-5-ylmethyl (S)-(4-(1- |
| | | | (dimethylcarbamoyl)piperidin-3-yl)phenyl)carbamate. |
| | | | LCMS-ESI (POS.) m/z: 373.1 [M + H]⁺. |
| | | | ¹H NMR (400 MHz, DMSO-d₆) δ 9.75 (s, 1H), 8.42 (s, |
| | | | 1H), 7.38 (d, J = 8.1 Hz, 2H), 7.31 (s, 1H), 7.18 (d, J = 8.4 |
| | | | Hz, 2H), 5.21 (s, 2H), 3.55 (dd, J = 22.6, 11.9 Hz, 2H), |
| | | | 2.73 (s, 8H), 1.87 (d, J = 11.2 Hz, 1H), 1.70 (d, J = 11.3 |
| | | | Hz, 1H), 1.57 (p, J = 13.9, 13.1 Hz, 3H). |
| 384 | 30.3 | dimethyl- sulfamoyl chloride | 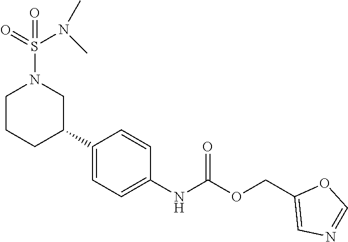 [SMILES: CN(C)S(=O)(=O)N1CCC[C@@H](c2ccc(NC(=O)OCc3cnco3)cc2)C1] |
| | | | Compound 384: oxazol-5-ylmethyl (S)-(4-(1-(N,N- |
| | | | dimethylsulfamoyl)piperidin-3-yl)phenyl)carbamate. |
| | | | LCMS-ESI (POS.) m/z: 409.1 [M + H]⁺. |
| | | | ¹H NMR (400 MHz, DMSO-d₆) δ 9.76 (s, 1H), 8.42 (s, |
| | | | 1H), 7.38 (d, J = 8.1 Hz, 2H), 7.30 (s, 1H), 7.20 (d, J = 8.3 |
| | | | Hz, 2H), 5.21 (s, 2H), 3.52 (dd, J = 11.7 Hz, 2H), 2.86 (t, |
| | | | J = 11.5 Hz, 2H), 2.74 (s, 6H), 2.71-2.61 (m, 1H), 1.88- |
| | | | 1.71 (m, 2H), 1.65-1.48 (m, 2H). |
| 383 | 30.3 | methyl carbono- chloridate | 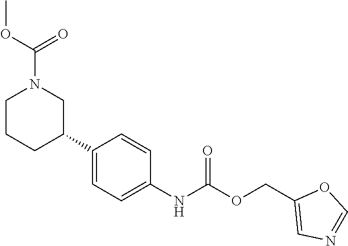 [SMILES: COC(=O)N1CCC[C@@H](c2ccc(NC(=O)OCc3cnco3)cc2)C1] |
| | | | Compound 383: methyl (S)-3-(4-(((oxazol-5- |
| | | | ylmethoxy)carbonyl)amino)phenyl)piperidine-1- |
| | | | carboxylate. |
| | | | LCMS-ESI (POS.) m/z: 360.1 [M + H]⁺ |
| | | | ¹H NMR (400 MHz, DMSO-d₆) δ 9.76 (s, 1H), 8.42 (s, |
| | | | 1H), 7.39 (d, J = 8.1 Hz, 2H), 7.31 (s, 1H), 7.18 (d, J = 8.3 |
| | | | Hz, 2H), 5.21 (s, 2H), 3.98 (s, 2H), 3.60 (s, 3H), 2.81 (s, |
| | | | 2H), 2.57 (d, J = 11.5 Hz, 1H), 1.86 (d, J = 12.8 Hz, 1H), |
| | | | 1.71 (d, J = 13.1 Hz, 1H), 1.62 (q, J = 11.7, 11.3 Hz, 1H), |
| | | | 1.45 (d, J = 12.8 Hz, 1H). |
| 382 | 30.3 | methane- sulfonyl chloride | 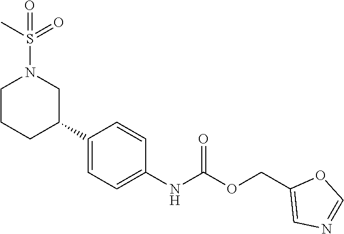 [SMILES: CS(=O)(=O)N1CCC[C@@H](c2ccc(NC(=O)OCc3cnco3)cc2)C1] |
| | | | Compound 382: oxazol-5-ylmethyl (S)-(4-(1- |
| | | | (methylsulfonyl)piperidin-3-yl)phenyl)carbamate. |
| | | | LCMS-ESI (POS.) m/z: 380.1 [M + H]⁺. |
| | | | ¹H NMR (400 MHz, DMSO-d₆) δ 9.77 (s, 1H), 8.43 (s, |
| | | | 1H), 7.40 (d, J = 8.2 Hz, 2H), 7.31 (s, 1H), 7.22 (d, J = 8.3 |
| | | | Hz, 2H), 5.22 (s, 2H), 3.59 (d, J = 12.2 Hz, 1H), 3.32 (s, |
| | | | 2H), 2.88 (s, 2H), 2.71 (dd, J = 13.8, 10.2 Hz, 3H), 1.84 (s, |
| | | | 1H), 1.89-1.79 (m, 1H), 1.59 (s, 1H), 1.54 (d, J = 11.3 |
| | | | Hz, 1H). |
| 381 | 30.3 | acetic anhydride | 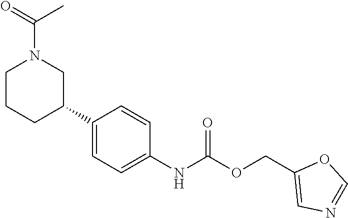 [SMILES: CC(=O)N1CCC[C@@H](c2ccc(NC(=O)OCc3cnco3)cc2)C1] |
| | | | Compound 381: oxazol-5-ylmethyl (S)-(4-(1- |
| | | | acetylpiperidin-3-yl)phenyl)carbamate. |
| | | | LCMS-ESI (POS.) m/z: 344.1 [M + H]⁺. |
| | | | ¹H NMR (400 MHz, DMSO-d₆) δ 9.75 (s, 1H), 8.42 (s, |
| | | | 1H), 7.44-7.34 (m, 2H), 7.30 (s, 1H), 7.20 (dd, J = 21.5, |
| | | | 8.2 Hz, 2H), 5.21 (s, 2H), 4.47-4.33 (m, 1H), 3.78 (dd, |
| | | | J = 30.2, 13.3 Hz, 1H), 3.10-2.96 (m, 1H), 2.71-2.55 (m, |
| | | | 1H), 2.01 (d, J = 5.6 Hz, 3H), 1.87 (d, J = 12.9 Hz, 1H), |
| | | | 1.79-1.56 (m, 2H), 1.56-1.29 (m, 2H). |
| 397 | 30.4 | oxetan-3-one | 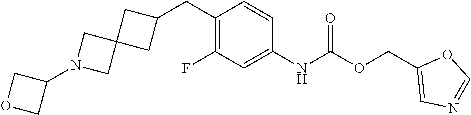 [SMILES: O=C(Nc1ccc(CC2CC3(C2)CN(C2COC2)C3)c(F)c1)OCc1cnco1] |
| | | | Compound 397: oxazol-5-ylmethyl (3-fluoro-4-((2- |
| | | | (oxetan-3-yl)-2-azaspiro[3.3]heptan-6- |
| | | | yl)methyl)phenyl)carbamate |
| | | | LCMS-ESI (POS.) m/z: 402.1 [M + H]⁺. |
| 472 | 30.4 | acetic anhydride | 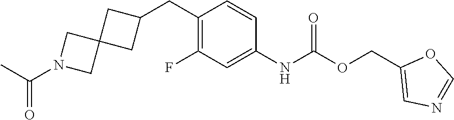 [SMILES: CC(=O)N1CC2(CC(Cc3ccc(NC(=O)OCc4cnco4)cc3F)C2)C1] |
| | | | Compound 472: oxazol-5-ylmethyl (4-((2-acetyl-2- |
| | | | azaspiro[3.3]heptan-6-yl)methyl)-3- |
| | | | fluorophenyl)carbamate |
| | | | LCMS-ESI (POS.) m/z: 388.1 [M + H]⁺ |
| | | | ¹H NMR (400 MHz, DMSO-d₆) δ 9.95 (s, 1H), 8.42 (s, |
| | | | 1H), 7.33-7.26 (m, 2H), 7.19-7.06 (m, 2H), 5.22 (s, |
| | | | 2H), 4.07 (s, 1H), 3.98 (s, 1H), 3.79 (s, 1H), 3.69 (s, 1H), |
| | | | 2.58 (d, J = 7.5 Hz, 2H), 2.37-2.27 (m, 1H), 2.24-2.12 |
| | | | (m, 2H), 1.88-1.80 (m, 2H), 1.69 (s, 3H). |
| 403 | 30.4 | isobutyryl chloride | 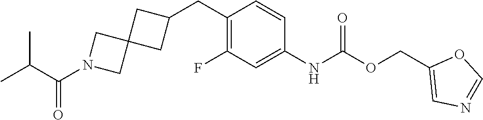 [SMILES: CC(C)C(=O)N1CC2(CC(Cc3ccc(NC(=O)OCc4cnco4)cc3F)C2)C1] |
| | | | Compound 403: oxazol-5-ylmethyl (3-fluoro-4-((2- |
| | | | isobutyryl-2-azaspiro[3.3]heptan-6- |
| | | | yl)methyl)phenyl)carbamate. |
| | | | LCMS-ESI (POS.) m/z: 416.1 [M + H]⁺ |
| | | | ¹H NMR (400 MHz, DMSO-d₆) δ 9.95 (s, 1H), 8.42 (s, |
| | | | 1H), 7.33-7.27 (m, 2H), 7.18-7.09 (m, 2H), 5.22 (s, |
| | | | 2H), 4.11 (s, 1H), 4.02 (s, 1H), 3.80 (s, 1H), 3.70 (s, 1H), |
| | | | 2.58 (d, J = 7.5 Hz, 2H), 2.41-2.35 (m, 1H), 2.34-2.27 |
| | | | (m, 1H), 2.24-2.12 (m, 2H), 1.89-1.80 (m, 2H), 0.93 (s, |
| | | | 3H), 0.92 (s, 3H). |
| 404 | 30.4 | methane- sulfonyl chloride | 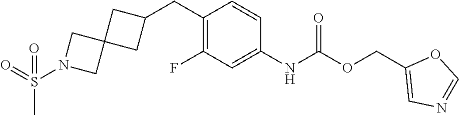 [SMILES: CS(=O)(=O)N1CC2(CC(Cc3ccc(NC(=O)OCc4cnco4)cc3F)C2)C1] |
| | | | Compound 404: oxazol-5-ylmethyl (3-fluoro-4-((2- |
| | | | (methylsulfonyl)-2-azaspiro[3.3]heptan-6- |
| | | | yl)methyl)phenyl)carbamate |
| | | | LCMS-ESI (POS.) m/z: 425.1 [M + H]⁺ |
| | | | ¹H NMR (400 MHz, DMSO-d₆) δ 9.95 (s, 1H), 8.43 (s, |
| | | | 1H), 7.33-7.26 (m, 2H), 7.18-7.10 (m, 2H), 5.22 (s, |
| | | | 2H), 3.84 (s, 2H), 3.75 (s, 2H), 3.30-3.28 (m, 1H), 2.92 |
| | | | (s, 3H), 2.69-2.65 (m, 1H), 2.58 (d, J = 7.5 Hz, 1H), 2.37- |
| | | | 2.27 (m, 2H), 2.25-2.15 (m, 1H), 1.89-1.81 (m, 1H). |
| 386 | 30.4 | ethanesulfonyl chloride | 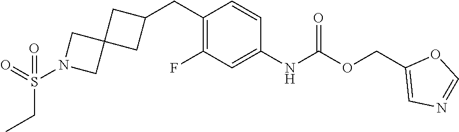 [SMILES: CCS(=O)(=O)N1CC2(CC(Cc3ccc(NC(=O)OCc4cnco4)cc3F)C2)C1] |
| | | | Compound 386: oxazol-5-ylmethyl (4-((2- |
| | | | (ethylsulfonyl)-2-azaspiro[3.3]heptan-6-yl)methyl)-3- |
| | | | fluorophenyl)carbamate |
| | | | LCMS-ESI (POS.) m/z: 438.1 [M + H]⁺ |
| | | | ¹H NMR (400 MHz, DMSO-d₆) δ 9.95 (s, 1H), 8.42 (s, |
| | | | 1H), 7.32-7.26 (m, 2H), 7.18-7.08 (m, 2H), 5.22 (s, |
| | | | 2H), 3.83 (s, 2H), 3.75 (s, 2H), 3.29 (s, 2H), 3.05 (q, J = |
| | | | 7.4 Hz, 3H), 2.66 (s, 1H), 2.57 (d, J = 7.5 Hz, 1H), 2.35- |
| | | | 2.30 (m, 2H), 1.18 (t, J = 7.4 Hz, 3H). |
| 402 | 30.4 | propionyl chloride | 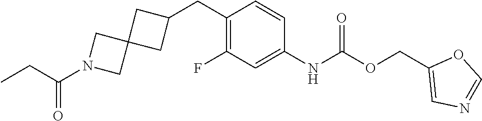 [SMILES: CCC(=O)N1CC2(CC(Cc3ccc(NC(=O)OCc4cnco4)cc3F)C2)C1] |
| | | | Compound 402: oxazol-5-ylmethyl (3-fluoro-4-((2- |
| | | | propionyl-2-azaspiro[3.3]heptan-6- |
| | | | yl)methyl)phenyl)carbamate |
| | | | LCMS-ESI (POS.) m/z: 402.1 [M + H]⁺ |
| | | | ¹H NMR (400 MHz, DMSO-d₆) δ 9.95 (s, 1H), 8.43 (s, |
| | | | 1H), 7.33-7.27 (m, 2H), 7.18-7.11 (m, 2H), 5.23 (s, |
| | | | 2H), 4.06 (s, 1H), 3.98 (s, 1H), 3.80 (s, 1H), 3.71 (s, 1H), |
| | | | 3.30 (s, 2H), 2.70-2.65 (s, 1H), 2.59 (d, J = 7.5 Hz, 2H), |
| | | | 2.35-2.31 (m, 1H), 2.23-2.14 (m, 1H), 1.99 (q, J = 7.5 |
| | | | Hz, 3H), 1.89-1.81 (m, 2H). |
| 399 | 30.4 | dimethyl- sulfamoyl chloride | 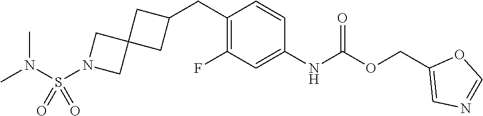 [SMILES: CN(C)S(=O)(=O)N1CC2(CC(Cc3ccc(NC(=O)OCc4cnco4)cc3F)C2)C1] |
| | | | Compound 399: oxazol-5-ylmethyl (4-((2-(N,N- |
| | | | dimethylsulfamoyl)-2-azaspiro[3.3]heptan-6- |
| | | | yl)methyl)-3-fluorophenyl)carbamate |
| | | | LCMS-ESI (POS.) m/z: 453.1 [M + H]⁺ |
| | | | ¹H NMR (400 MHz, DMSO-d₆) δ 9.95 (s, 1H), 8.42 (s, |
| | | | 1H), 7.34-7.26 (m, 2H), 7.18-7.09 (m, 2H), 5.22 (s, |
| | | | 2H), 3.78 (s, 2H), 3.69 (s, 2H), 2.72-2.64z (m, 6H), 2.57 |
| | | | (d, J = 7.5 Hz, 2H), 2.35-2.27 (m, 1H), 2.23-2.14 (m, |
| | | | 2H), 1.84 (t, J = 9.8 Hz, 2H). |
| 398 | 30.4 | dimethyl- carbamoyl chloride | 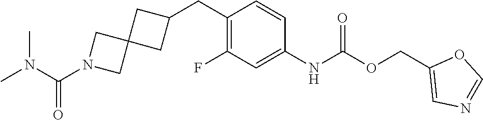 [SMILES: CN(C)C(=O)N1CC2(CC(Cc3ccc(NC(=O)OCc4cnco4)cc3F)C2)C1] |
| | | | Compound 398: oxazol-5-ylmethyl (4-((2- |
| | | | (dimethylcarbamoyl)-2-azaspiro[3.3]heptan-6- |
| | | | yl)methyl)-3-fluorophenyl)carbamate |
| | | | LCMS-ESI (POS.) m/z: 417.1 [M + H]⁺ |
| | | | ¹H NMR (400 MHz, DMSO-d₆) δ 9.95 (s, 1H), 8.42 (s, |
| | | | 1H), 7.33-7.26 (m, 2H), 7.19-7.09 (m, 2H), 5.76 (s, |
| | | | 1H), 5.22 (s, 2H), 3.84 (s, 2H), 3.75 (s, 2H), 2.67 (s, 1H), |
| | | | 2.57 (d, J = 7.5 Hz, 2H), 2.36-2.24 (m, 2H), 2.16 (t, J = |
| | | | 9.9 Hz, 3H), 1.86-1.75 (m, 3H), 1.23 (s, 1H). |
| 396 | 30.4 | methyl carbono- chloridate | 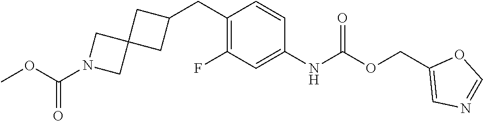 [SMILES: COC(=O)N1CC2(CC(Cc3ccc(NC(=O)OCc4cnco4)cc3F)C2)C1] |
| | | | Compound 396: methyl 6-(2-fluoro-4-(((oxazol-5- |
| | | | ylmethoxy)carbonyl)amino)benzyl)-2- |
| | | | azaspiro[3.3]heptane-2-carboxylate |
| | | | LCMS-ESI (POS.) m/z: 404.1 [M + H]⁺ |
| | | | ¹H NMR (400 MHz, DMSO-d₆) δ 9.94 (s, 1H), 8.42 (s, |
| | | | 1H), 7.33-7.26 (m, 2H), 7.16-7.08 (m, 2H), 5.22 (s, |
| | | | 2H), 3.88 (s, 2H), 3.78 (s, 2H), 3.51 (s, 4H), 3.29 (s, 2H), |
| | | | 2.67 (s, 2H), 3.35-2.25 (m, 2H). |
| 401 | 30.4-a | acetic anhydride | 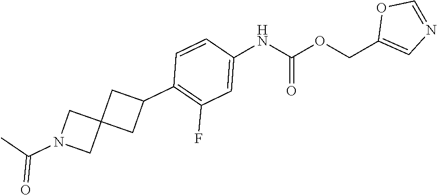 [SMILES: CC(=O)N1CC2(CC(c3ccc(NC(=O)OCc4cnco4)cc3F)C2)C1] |
| | | | Compound 401: oxazol-5-ylmethyl (4-(2-acetyl-2- |
| | | | azaspiro[3.3]heptan-6-yl)-3-fluorophenyl)carbamate |
| | | | LCMS-ESI (POS.) m/z: 404.1 [M + H]⁺ |
| | | | ¹H NMR (400 MHz, DMSO-d₆) δ 9.97 (s, 1H), 8.42 (s, |
| | | | 1H), 7.31 (s, 1H), 7.30-7.15 (m, 3H), 5.22 (s, 2H), 4.23 |
| | | | (s, 1H), 4.01 (s, 1H), 3.95 (s, 1H), 3.73 (s, 1H), 3.45 |
| | | | (p, J = 8.9 Hz, 1H), 2.56-2.50 (m, 2H), 2.30-2.22 (m, |
| | | | 2H), 1.73 (d, J = 10.8 Hz, 3H). |
| 400 | 30.4-a | dimethyl- carbamoyl chloride | 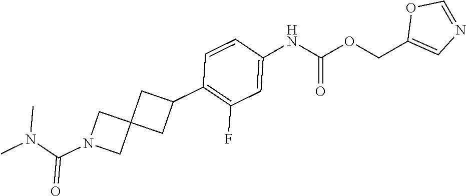 [SMILES: CN(C)C(=O)N1CC2(CC(c3ccc(NC(=O)OCc4cnco4)cc3F)C2)C1] |
| | | | Compound 400: oxazol-5-ylmethyl (4-(2- |
| | | | (dimethylcarbamoyl)-2-azaspiro[3.3]heptan-6-yl)-3- |
| | | | fluorophenyl)carbamate |
| | | | LCMS-ESI (POS.) m/z: 403.1 [M + H]⁺ |
| | | | ¹H NMR (400 MHz, DMSO-d₆) δ 9.97 (s, 1H), 8.43 (s, |
| | | | 1H), 7.31 (s, 1H), 7.31-7.14 (m, 3H), 5.23 (s, 2H), 4.01 |
| | | | (s, 2H), 3.79 (s, 2H), 3.45 (p, J = 8.8 Hz, 1H), 2.74 (s, 6H), |
| | | | 2.48-2.45 (m, 2H), 2.27-2.20 (m, 2H). |
| 388 | 30.4-a | methane- sulfonyl chloride | 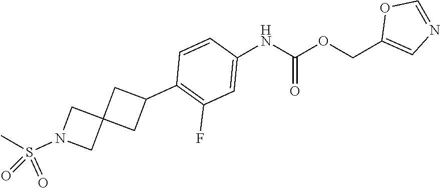 [SMILES: CS(=O)(=O)N1CC2(CC(c3ccc(NC(=O)OCc4cnco4)cc3F)C2)C1] |
| | | | Compound 388: oxazol-5-ylmethyl (3-fluoro-4-(2- |
| | | | (methylsulfonyl)-2-azaspiro[3.3]heptan-6- |
| | | | yl)phenyl)carbamate |
| | | | LCMS-ESI (POS.) m/z: 410.1 [M + H]⁺ |
| | | | ¹H NMR (400 MHz, DMSO-d₆) δ 9.97 (s, 1H), 8.42 (s, |
| | | | 1H), 7.31 (s, 1H), 7.30-7.14 (m, 3H), 5.22 (s, 2H), 4.00 |
| | | | (s, 2H), 3.78 (s, 2H), 3.46 (p, J = 8.8 Hz, 1H), 2.96 (s, 3H), |
| | | | 2.57-2.52 (m, 2H), 2.27 (td, J = 9.7, 2.8 Hz, 2H). |
| 387 | 30.4-a | dimethyl- sulfamoyl chloride | 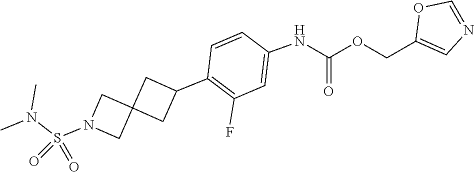 [SMILES: CN(C)S(=O)(=O)N1CC2(CC(c3ccc(NC(=O)OCc4cnco4)cc3F)C2)C1] |
| | | | Compound 387: oxazol-5-ylmethyl (4-(2-(N,N- |
| | | | dimethylsulfamoyl)-2-azaspiro[3.3]heptan-6-yl)-3- |
| | | | fluorophenyl)carbamate |
| | | | LCMS-ESI (POS.) m/z: 439.1 [M + H]⁺ |
| | | | ¹H NMR (400 MHz, DMSO-d₆) δ 9.96 (s, 1H), 8.42 (s, |
| | | | 1H), 7.31 (s, 1H), 7.30-7.12 (m, 3H), 5.22 (s, 2H), 3.95 |
| | | | (s, 2H), 3.72 (s, 2H), 3.45 (p, J = 9.0 Hz, 1H), 2.72 (s, 6H), |
| | | | 2.56-2.51 (m, 2H), 2.30-2.23 (m, 2H). |
| 405 | 30.5 | dimethyl- sulfamoyl chloride | 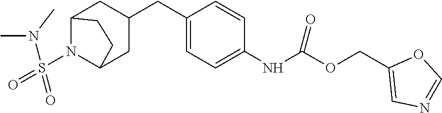 [SMILES: CN(C)S(=O)(=O)N1C2CCC1CC(Cc1ccc(NC(=O)OCc3cnco3)cc1)C2] |
| | | | Compound 405: oxazol-5-ylmethyl (4-((8-(N,N- |
| | | | dimethylsulfamoyl)-8-azabicyclo[3.2.1]octan-3- |
| | | | yl)methyl)phenyl)carbamate |
| | | | LCMS-ESI (POS.) m/z: 449.1 [M + H]⁺ |
| | | | ¹H NMR (400 MHz, Methanol-d₄) δ 8.24 (s, 1H), 7.35 (t, |
| | | | J = 7.4 Hz, 2H), 7.25 (s, 1H), 7.11 (t, J = 8.4 Hz, 2H), 5.25 |
| | | | (s, 2H), 4.12-3.95 (m, 2H), 2.79 (d, J = 4.5 Hz, 6H), 2.72 |
| | | | (d, J = 7.5 Hz, 1H), 2.48 (d, J = 7.1 Hz, 1H), 2.26-1.97 |
| | | | (m, 4H), 1.91 (t, J = 7.1 Hz, 1H), 1.69 (t, J = 7.0 Hz, 1H), |
| | | | 1.66-1.56 (m, 1H), 1.56-1.39 (m, 2H). |
| 406 | 30.5 | methane- sulfonyl chloride | 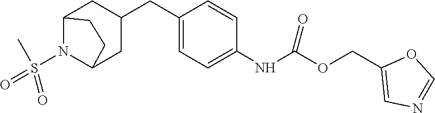 [SMILES: CS(=O)(=O)N1C2CCC1CC(Cc1ccc(NC(=O)OCc3cnco3)cc1)C2] |
| | | | Compound 406: oxazol-5-ylmethyl (4-((8- |
| | | | (methylsulfonyl)-8-azabicyclo[3.2.1]octan-3- |
| | | | yl)methyl)phenyl)carbamate |
| | | | LCMS-ESI (POS.) m/z: 420.1 [M + H]⁺ |
| 407 | 30.5 | methyl carbono- chloridate | 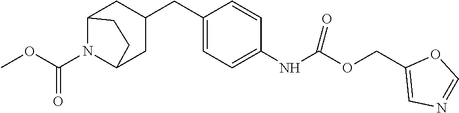 [SMILES: COC(=O)N1C2CCC1CC(Cc1ccc(NC(=O)OCc3cnco3)cc1)C2] |
| | | | Compound 407: methyl 3-(4-(((oxazol-5- |
| | | | ylmethoxy)carbonyl)amino)benzyl)-8- |
| | | | azabicyclo[3.2.1]octane-8-carboxylate |
| | | | LCMS-ESI (POS.) m/z: 400.1 [M + H]⁺ |
| 408 | 30.5 | dimethyl- carbamoyl chloride | 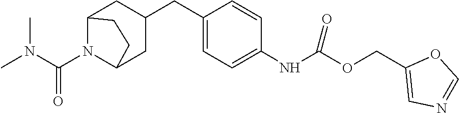 [SMILES: CN(C)C(=O)N1C2CCC1CC(Cc1ccc(NC(=O)OCc3cnco3)cc1)C2] |
| | | | Compound 408: oxazol-5-ylmethyl (4-((8- |
| | | | (dimethylcarbamoyl)-8-azabicyclo[3.2.1]octan-3- |
| | | | yl)methyl)phenyl)carbamate |
| | | | LCMS-ESI (POS.) m/z: 413.1 [M + H]⁺ |
| 409 | 30.5 | isobutyryl chloride | 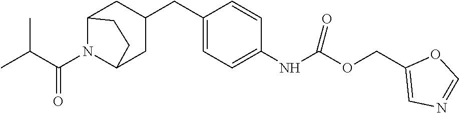 [SMILES: CC(C)C(=O)N1C2CCC1CC(Cc1ccc(NC(=O)OCc3cnco3)cc1)C2] |
| | | | Compound 409: oxazol-5-ylmethyl (4-((8-isobutyryl-8- |
| | | | azabicyclo[3.2.1]octan-3-yl)methyl)phenyl)carbamate |
| | | | LCMS-ESI (POS.) m/z: 412.1 [M + H]⁺ |
| 410 | 30.5 | acetic anhydride | 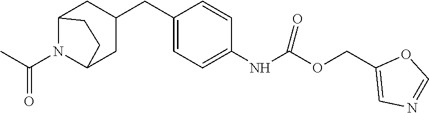 [SMILES: CC(=O)N1C2CCC1CC(Cc1ccc(NC(=O)OCc3cnco3)cc1)C2] |
| | | | Compound 410: oxazol-5-ylmethyl (4-((8-acetyl-8- |
| | | | azabicyclo[3.2.1]octan-3-yl)methyl)phenyl)carbamate |
| | | | LCMS-ESI (POS.) m/z: 384.1 [M + H]⁺ |
| 389 | 30.5 | morpholine-4- carbonyl chloride | 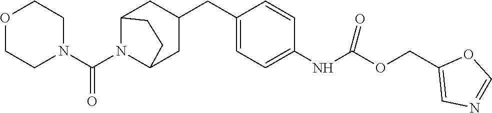 [SMILES: O=C(Nc1ccc(CC2CC3CCC(C2)N3C(=O)N2CCOCC2)cc1)OCc1cnco1] |
| | | | Compound 389: oxazol-5-ylmethyl (4-((8-(morpholine- |
| | | | 4-carbonyl)-8-azabicyclo[3.2.1]octan-3- |
| | | | yl)methyl)phenyl)carbamate |
| | | | LCMS-ESI (POS.) m/z: 455.1[M + H]⁺ |
| 390 | 30.5 | piperidine-1- carbonyl chloride | 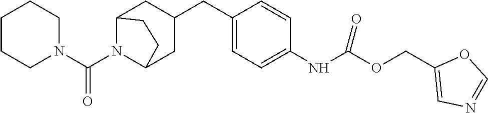 [SMILES: O=C(Nc1ccc(CC2CC3CCC(C2)N3C(=O)N2CCCCC2)cc1)OCc1cnco1] |
| | | | Compound 390: oxazol-5-ylmethyl (4-((8-(piperidine-1- |
| | | | carbonyl)-8-azabicyclo[3.2.1]octan-3- |
| | | | yl)methyl)phenyl)carbamate |
| | | | LCMS-ESI (POS.) m/z: 453.1[M + H]⁺ |
| 391 | 30.5 | pyrrolidine-1- carbonyl chloride | 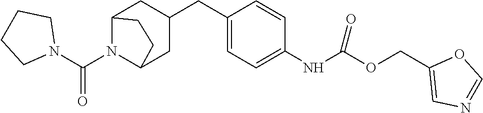 [SMILES: O=C(Nc1ccc(CC2CC3CCC(C2)N3C(=O)N2CCCC2)cc1)OCc1cnco1] |
| | | | Compound 391: oxazol-5-ylmethyl (4-((8-(pyrrolidine- |
| | | | 1-carbonyl)-8-azabicyclo[3.2.1]octan-3- |
| | | | yl)methyl)phenyl)carbamate |
| | | | LCMS-ESI (POS.) m/z: 439.1[M + H]⁺ |
| 392 | 30.5 | azetidine-1- carbonyl chloride | 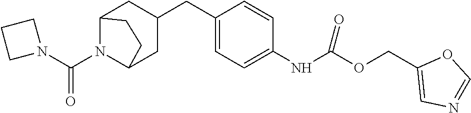 [SMILES: O=C(Nc1ccc(CC2CC3CCC(C2)N3C(=O)N2CCC2)cc1)OCc1cnco1] |
| | | | Compound 392: oxazol-5-ylmethyl (4-((8-(azetidine-1- |
| | | | carbonyl)-8-azabicyclo[3.2.1]octan-3- |
| | | | yl)methyl)phenyl)carbamate |
| | | | LCMS-ESI (POS.) m/z: 425.1[M + H]⁺ |
| 393 | 30.5 | azetidine-1- sulfonyl chloride | 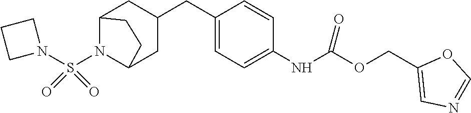 [SMILES: O=C(Nc1ccc(CC2CC3CCC(C2)N3S(=O)(=O)N2CCC2)cc1)OCc1cnco1] |
| | | | Compound 393: oxazol-5-ylmethyl (4-((8-(azetidin-1- |
| | | | ylsulfonyl)-8-azabicyclo[3.2.1]octan-3- |
| | | | yl)methyl)phenyl)carbamate |
| | | | LCMS-ESI (POS.) m/z: 461.1[M + H]⁺ |
| 394 | 30.5 | cyclobutane- sulfonyl chloride | 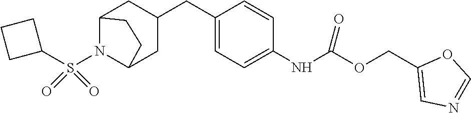 [SMILES: O=C(Nc1ccc(CC2CC3CCC(C2)N3S(=O)(=O)C2CCC2)cc1)OCc1cnco1] |
| | | | Compound 394: oxazol-5-ylmethyl (4-((8- |
| | | | (cyclobutylsulfonyl)-8-azabicyclo[3.2.1]octan-3- |
| | | | yl)methyl)phenyl)carbamate |
| | | | LCMS-ESI (POS.) m/z: 460.1[M + H]⁺ |
| 395 | 30.5 | cyclopropane- sulfonyl chloride | 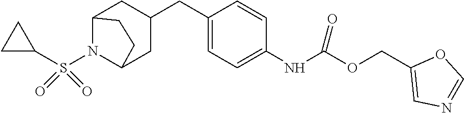 [SMILES: O=C(Nc1ccc(CC2CC3CCC(C2)N3S(=O)(=O)C2CC2)cc1)OCc1cnco1] |
| | | | Compound 395: oxazol-5-ylmethyl (4-((8- |
| | | | (cyclopropylsulfonyl)-8-azabicyclo[3.2.1]octan-3- |
| | | | yl)methyl)phenyl)carbamate |
| | | | LCMS-ESI (POS.) m/z: 446.1[M + H]⁺ |
| | | | ¹H NMR (400 MHz, Methanol-d₄) δ 8.24 (s, 1H), 7.35 (t, |
| | | | J = 7.7 Hz, 2H), 7.11 (t, J = 9.0 Hz, 2H), 5.25 (s, 2H), 4.16 |
| | | | (q, J = 3.4 Hz, 2H), 2.73 (d, J = 7.3 Hz, 1H), 2.57-2.38 |
| | | | (m, 2H), 2.26-1.87 (m, 5H), 1.80-1.70 (m, 1H), 1.70- |
| | | | 1.57 (m, 2H), 1.48 (td, J = 12.7, 2.7 Hz, 2H), 1.14-0.86 |
| | | | (m, 4H). |
| 411 | 30.6 | dimethyl- carbamoyl chloride | 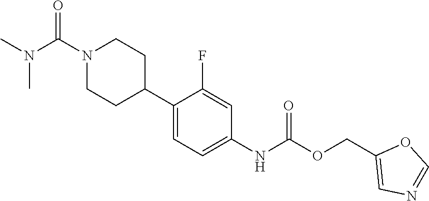 [SMILES: CN(C)C(=O)N1CCC(c2ccc(NC(=O)OCc3cnco3)cc2F)CC1] |
| | | | Compound 411: oxazol-5-ylmethyl (4-(1- |
| | | | (dimethylcarbamoyl)piperidin-4-yl)-3- |
| | | | fluorophenyl)carbamate |
| | | | LCMS-ESI (POS.) m/z: 391.1[M + H]⁺ |
| 412 | 30.6 | propionyl chloride | 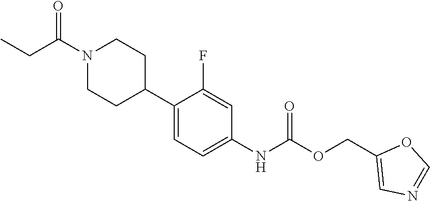 [SMILES: CCC(=O)N1CCC(c2ccc(NC(=O)OCc3cnco3)cc2F)CC1] |
| | | | Compound 412: oxazol-5-ylmethyl (3-fluoro-4-(1- |
| | | | propionylpiperidin-4-yl)phenyl)carbamate |
| | | | LCMS-ESI (POS.) m/z: 376.1[M + H]⁺ |
| 413 | 30.6 | acetic anhydride | 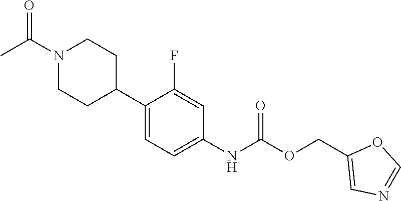 [SMILES: CC(=O)N1CCC(c2ccc(NC(=O)OCc3cnco3)cc2F)CC1] |
| | | | Compound 413: oxazol-5-ylmethyl (4-(1- |
| | | | acetylpiperidin-4-yl)-3-fluorophenyl)carbamate |
| | | | LCMS-ESI (POS.) m/z: 362.1[M + H]⁺ |
| 414 | 30.6 | isobutyryl chloride | 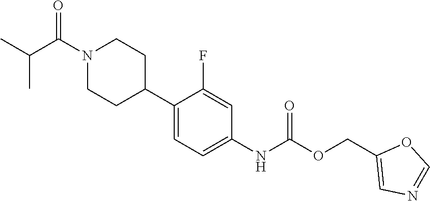 [SMILES: CC(C)C(=O)N1CCC(c2ccc(NC(=O)OCc3cnco3)cc2F)CC1] |
| | | | Compound 414: oxazol-5-ylmethyl (3-fluoro-4-(1- |
| | | | isobutyrylpiperidin-4-yl)phenyl)carbamate |
| | | | LCMS-ESI (POS.) m/z: 390.1[M + H]⁺ |
| | | | ¹H NMR (400 MHz, DMSO-d₆) δ 9.97 (s, 1H), 8.42 (s, |
| | | | 1H), 7.31 (d, J = 6.1 Hz, 2H), 7.23 (t, J = 8.5 Hz, 1H), |
| | | | 7.18-7.11 (m, 1H), 5.22 (s, 2H), 4.55 (d, J = 12.9 Hz, |
| | | | 1H), 4.04 (d, J = 13.2 Hz, 1H), 3.12 (t, J = 12.9 Hz, 1H), |
| | | | 3.04-2.83 (m, 2H), 2.58 (t, J = 12.4 Hz, 1H), 1.74 (t, J = |
| | | | 17.4 Hz, 2H), 1.63-1.37 (m, 2H), 1.00 (dd, J = 10.9, 6.6 |
| | | | Hz, 6H). |
| 415 | 30.6 | methane- sulfonyl chloride | 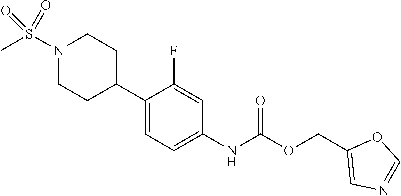 [SMILES: CS(=O)(=O)N1CCC(c2ccc(NC(=O)OCc3cnco3)cc2F)CC1] |
| | | | Compound 415: oxazol-5-ylmethyl (3-fluoro-4-(1- |
| | | | (methylsulfonyl)piperidin-4-yl)phenyl)carbamate |
| | | | LCMS-ESI (POS.) m/z: 398.1[M + H]⁺ |
| 416 | 30.6 | dimethyl- sulfamoyl chloride | 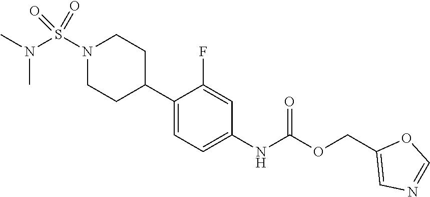 [SMILES: CN(C)S(=O)(=O)N1CCC(c2ccc(NC(=O)OCc3cnco3)cc2F)CC1] |
| | | | Compound 416: oxazol-5-ylmethyl (4-(1-(N,N- |
| | | | dimethylsulfamoyl)piperidin-4-yl)-3- |
| | | | fluorophenyl)carbamate |
| | | | LCMS-ESI (POS.) m/z: 427.1[M + H]⁺ |
| 417 | 30.6 | methyl carbono- chloridate | 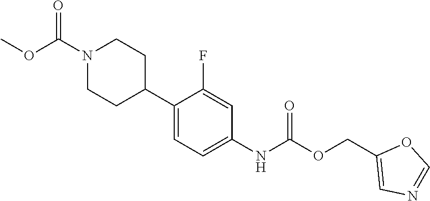 [SMILES: COC(=O)N1CCC(c2ccc(NC(=O)OCc3cnco3)cc2F)CC1] |
| | | | Compound 417: methyl 4-(2-fluoro-4-(((oxazol-5- |
| | | | ylmethoxy)carbonyl)amino)phenyl)piperidine-1- |
| | | | carboxylate |
| | | | LCMS-ESI (POS.) m/z: 378.1[M + H]⁺ |
| 444 | 30.7 | acetic anhydride | 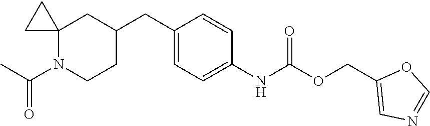 [SMILES: CC(=O)N1CCC(Cc2ccc(NC(=O)OCc3cnco3)cc2)CC12CC2] |
| | | | Compound 444: oxazol-5-ylmethyl (4-((4-acetyl-4- |
| | | | azaspiro[2.5]octan-7-yl)methyl)phenyl)carbamate |
| | | | LCMS-ESI (POS.) m/z: 384.1[M + H]⁺ |
| 443 | 30.7 | methyl carbono- chloridate | 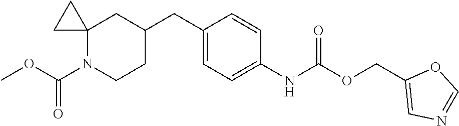 [SMILES: COC(=O)N1CCC(Cc2ccc(NC(=O)OCc3cnco3)cc2)CC12CC2] |
| | | | Compound 443: methyl 7-(4-(((oxazol-5- |
| | | | ylmethoxy)carbonyl)amino)benzyl)-4- |
| | | | azaspiro[2.5]octane-4-carboxylate |
| | | | LCMS-ESI (POS.) m/z: 400.1[M + H]⁺ |
| | | | ¹H NMR (400 MHz, DMSO-d₆) δ 9.70 (s, 1H), 8.41 (s, |
| | | | 1H), 7.33 (d, J = 8.1 Hz, 2H), 7.30 (s, 1H), 7.06 (d, J = 8.5 |
| | | | Hz, 2H), 5.20 (s, 2H), 3.87 (d, J = 15.2 Hz, 1H), 3.57 (s, |
| | | | 3H), 2.81 (t, J = 12.6, 2.0 Hz, 1H), 2.46-2.36 (m, 2H), |
| | | | 1.95-1.82 (m, 1H), 1.57-1.44 (m, 2H), 1.13-0.95 (m, |
| | | | 2H), 0.86 (dd, J = 13.0, 3.8 Hz, 1H), 0.78 (dt, J = 9.6, 6.2 |
| | | | Hz, 1H), 0.48-0.40 (m, 1H), 0.36-0.28 (m, 1H). |
| 442 | 30.7 | dimethyl- carbamoyl chloride | 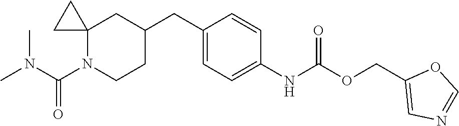 [SMILES: CN(C)C(=O)N1CCC(Cc2ccc(NC(=O)OCc3cnco3)cc2)CC12CC2] |
| | | | Compound 442: oxazol-5-ylmethyl (4-((4- |
| | | | (dimethylcarbamoyl)-4-azaspiro[2.5]octan-7- |
| | | | yl)methyl)phenyl)carbamate |
| | | | LCMS-ESI (POS.) m/z: 413.1[M + H]⁺ |
| | | | ¹H NMR (400 MHz, DMSO-d₆) δ 9.70 (s, 1H), 8.41 (s, |
| | | | 1H), 7.33 (d, J = 8.1 Hz, 2H), 7.29 (s, 1H), 7.06 (d, J = 8.5 |
| | | | Hz, 2H), 5.20 (s, 2H), 3.41 (ddd, J = 13.1, 4.1, 2.2 Hz, |
| | | | 1H), 2.87 (td, J = 13.3, 2.3 Hz, 1H), 2.68 (s, 6H), 2.44- |
| | | | 2.36 (m, 2H), 1.88-1.75 (m, 2H), 1.49 (d, J = |
| | | | 12.7 Hz, 1H), 0.97 (qd, J = 12.3, 4.1 Hz, 1H), 0.86-0.74 |
| | | | (m, 1H), 0.69-0.58 (m, 2H), 0.36-0.29 (m, 1H), 0.27- |
| | | | 0.20 (ddd, J = 9.2, 6.5, 4.1 Hz, 1H). |
| 419 | 30.7 | methane- sulfonyl chloride | 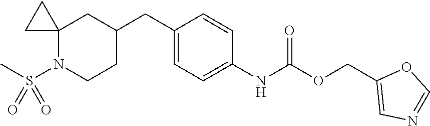 [SMILES: CS(=O)(=O)N1CCC(Cc2ccc(NC(=O)OCc3cnco3)cc2)CC12CC2] |
| | | | Compound 419: oxazol-5-ylmethyl (4-((4- |
| | | | (methylsulfonyl)-4-azaspiro[2.5]octan-7- |
| | | | yl)methyl)phenyl)carbamate |
| | | | LCMS-ESI (POS.) m/z: 420.1[M + H]⁺ |
| | | | ¹H NMR (400 MHz, DMSO-d₆) δ 9.71 (s, 1H), 8.41 (s, |
| | | | 1H), 7.34 (d, J = 8.1 Hz, 2H), 7.30 (s, 1H), 7.07 (d, J = 8.2 |
| | | | Hz, 2H), 5.20 (s, 2H), 3.79-3.70 (m, 1H), 2.97-2.88 (m, |
| | | | 1H), 2.94 (s, 3H), 2.47-2.38 (m, 2H), 1.94-1.82 (m, |
| | | | 1H), 1.71 (t, J = 12.4 Hz, 1H), 1.54-1.47 (m, 1H), 1.44- |
| | | | 1.37 (m, 1H), 1.29 (qd, J = 12.9, 3.8 Hz, 1H), 0.88-0.80 |
| | | | (m, 1H), 0.79-0.71 (m, 1H), 0.52-0.44 (m, 1H), 0.40- |
| | | | 0.32 (m, 1H). |
| 418 | 30.7 | ethanesulfonyl chloride | 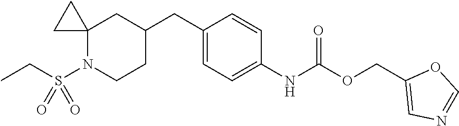 [SMILES: CCS(=O)(=O)N1CCC(Cc2ccc(NC(=O)OCc3cnco3)cc2)CC12CC2] |
| | | | Compound 418: oxazol-5-ylmethyl (4-((4- |
| | | | (ethylsulfonyl)-4-azaspiro[2.5]octan-7- |
| | | | yl)methyl)phenyl)carbamate |
| | | | LCMS-ESI (POS.) m/z: 434.1[M + H]⁺ |
| | | | ¹H NMR (400 MHz, DMSO-d₆) δ 9.71 (s, 1H), 8.41 (s, |
| | | | 1H), 7.33 (d, J = 8.1 Hz, 2H), 7.30 (s, 1H), 7.07 (d, J = 8.3 |
| | | | Hz, 2H), 5.20 (s, 2H), 3.69-2.61 (m, 1H), 3.03-2.89 (m, |
| | | | 3H), 2.46-2.40 (m, 2H), 1.93-1.81 (m, 1H), 1.67 (t, J = |
| | | | 12.3 Hz, 1H), 1.56-1.48 (m, 1H), 1.35-1.19 (m, 2H), |
| | | | 1.09 (t, J = 7.3 Hz, 3H), 0.87 (dd, J = 13.4, 2.9 Hz 1H), |
| | | | 0.74 (dt, J = 9.6, 6.5 Hz, 1H), 0.51-0.45 (m, 1H), 0.40- |
| | | | 0.33 (m, 1H). |
| 440 | 30.7 | dimethyl- sulfamoyl chloride | 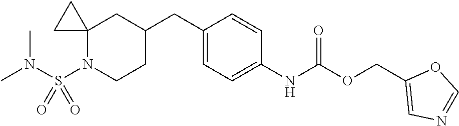 [SMILES: CN(C)S(=O)(=O)N1CCC(Cc2ccc(NC(=O)OCc3cnco3)cc2)CC12CC2] |
| | | | Compound 440: oxazol-5-ylmethyl (4-((4-(N,N- |
| | | | dimethylsulfamoyl)-4-azaspiro[2.5]octan-7- |
| | | | yl)methyl)phenyl)carbamate |
| | | | LCMS-ESI (POS.) m/z: 449.1[M + H]⁺ |
| | | | ¹H NMR (400 MHz, DMSO-d₆) δ 9.71 (s, 1H), 8.41 (s, |
| | | | 1H), 7.33 (d, J = 8.1 Hz, 2H), 7.30 (s, 1H), 7.07 (d, J = 8.5 |
| | | | Hz, 2H), 5.20 (s, 2H), 3.61-3.53 (m, 1H), 3.00-2.91 (m, |
| | | | 1H), 2.58 (s, 6H), 2.46-2.40 (m, 2H), 1.92-1.82 (m, |
| | | | 1H), 1.73 (td, J = 11.8, 1.3 Hz, 1H), 1.51-1.43 (m, 1H), |
| | | | 1.37 (td, J = 12.5, 4.2 Hz, 1H), 1.22 (dt, J = 10.2, 5.8 Hz, |
| | | | 1H), 0.87-0.81 (m, 1H), 0.71 (dt, J = 9.6, 6.2 Hz, 1H), |
| | | | 0.55-047 (m, 1H), 0.40-0.32 (m, 1H). |
| 420 | 426 | methane- sulfonyl chloride | 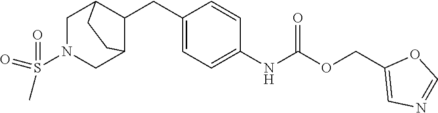 [SMILES: CS(=O)(=O)N1CC2CCC(C1)C2Cc1ccc(NC(=O)OCc2cnco2)cc1] |
| | | | Compound 420: oxazol-5-ylmethyl (4-((3- |
| | | | (methylsulfonyl)-3-azabicyclo[3.2.1]octan-8- |
| | | | yl)methyl)phenyl)carbamate |
| | | | LCMS-ESI (POS.) m/z: 420.1[M + H]⁺ |
| 421 | 426 | methyl carbono- chloridate | 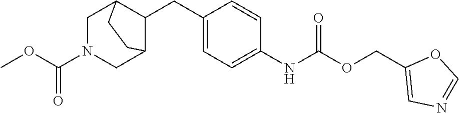 [SMILES: COC(=O)N1CC2CCC(C1)C2Cc1ccc(NC(=O)OCc2cnco2)cc1] |
| | | | Compound 421: methyl 8-(4-(((oxazol-5- |
| | | | ylmethoxy)carbonyl)amino)benzyl)-3- |
| | | | azabicyclo[3.2.1]octane-3-carboxylate |
| | | | LCMS-ESI (POS.) m/z: 400.1[M + H]⁺ |
| 422 | 426 | dimethyl- carbamoyl chloride | 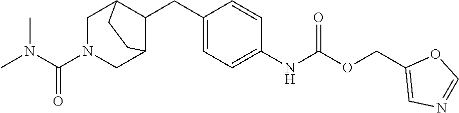 [SMILES: CN(C)C(=O)N1CC2CCC(C1)C2Cc1ccc(NC(=O)OCc2cnco2)cc1] |
| | | | Compound 422: oxazol-5-ylmethyl (4-((3- |
| | | | (dimethylcarbamoyl)-3-azabicyclo[3.2.1]octan-8- |
| | | | yl)methyl)phenyl)carbamate |
| | | | LCMS-ESI (POS.) m/z: 413.1[M + H]⁺ |
| | | | ¹H NMR (400 MHz, Methanol-d₄) δ 8.24 (s, 1H), 7.36 (d, |
| | | | J = 8.1 Hz, 2H), 7.25 (s, 1H), 7.19 (d, J = 8.5 Hz, 2H), |
| | | | 5.25 (s, 2H), 3.51-3.39 (m, 4H), 2.96 (d, J = 7.4 Hz, 2H), |
| | | | 2.81 (s, 6H), 2.04-1.91 (m, 3H), 1.78-1.56 (m, 4H). |
| 423 | 426 | isobutyryl chloride | 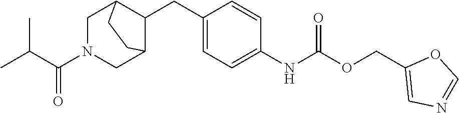 [SMILES: CC(C)C(=O)N1CC2CCC(C1)C2Cc1ccc(NC(=O)OCc2cnco2)cc1] |
| | | | Compound 423: oxazol-5-ylmethyl (4-((3-isobutyryl-3- |
| | | | azabicyclo[3.2.1]octan-8-yl)methyl)phenyl)carbamate |
| | | | LCMS-ESI (POS.) m/z: 412.1[M + H]⁺ |
| 424 | 426 | acetic anhydride | 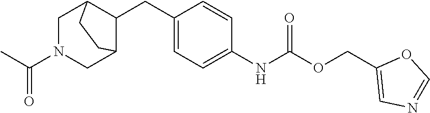 [SMILES: CC(=O)N1CC2CCC(C1)C2Cc1ccc(NC(=O)OCc2cnco2)cc1] |
| | | | Compound 424: oxazol-5-ylmethyl (4-((3-acetyl-3- |
| | | | azabicyclo[3.2.1]octan-8-yl)methyl)phenyl)carbamate |
| | | | LCMS-ESI (POS.) m/z: 384.1[M + H]⁺ |
| | | | ¹H NMR (400 MHz, Methanol-d₄) δ 8.24 (s, 1H), 7.37 (d, |
| | | | J = 8.1 Hz, 2H), 7.25 (s, 1H), 7.25-7.15 (m, 2H), 5.25 (s, |
| | | | 2H), 4.08-3.92 (m, 1H), 3.66 (d, J = 12.7 Hz, 1H), 3.47 |
| | | | (dd, J = 12.7, 2.8 Hz, 1H), 3.17 (d, J = 13.3 Hz, 1H), 2.92 |
| | | | (d, J = 6.9 Hz, 2H), 2.11 (s, 3H), 2.10-1.97 (m, 3H), 1.88- |
| | | | 1.71 (m, 2H), 1.65-1.45 (m, 2H). |
| 427 | 30.9 | ethanesulfonyl chloride | 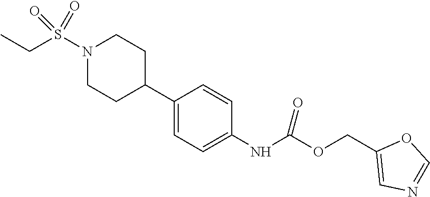 [SMILES: CCS(=O)(=O)N1CCC(c2ccc(NC(=O)OCc3cnco3)cc2)CC1] |
| | | | Compound 427: oxazol-5-ylmethyl (4-(1- |
| | | | (ethylsulfonyl)piperidin-4-yl)phenyl)carbamate |
| | | | LCMS-ESI (POS.) m/z: 394.1[M + H]⁺ |
| 428 | 30.9 | azetidine-1- sulfonyl chloride | 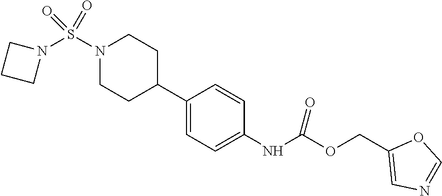 [SMILES: O=C(Nc1ccc(C2CCN(S(=O)(=O)N3CCC3)CC2)cc1)OCc1cnco1] |
| | | | Compound 428: oxazol-5-ylmethyl (4-(1-(azetidin-1- |
| | | | ylsulfonyl)piperidin-4-yl)phenyl)carbamate |
| | | | LCMS-ESI (POS.) m/z: 421.1[M + H]⁺ |
| 429 | 30.9 | azetidine-1- carbonyl chloride | 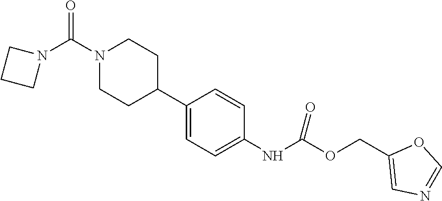 [SMILES: O=C(Nc1ccc(C2CCN(C(=O)N3CCC3)CC2)cc1)OCc1cnco1] |
| | | | Compound 429: oxazol-5-ylmethyl (4-(1-(azetidine-1- |
| | | | carbonyl)piperidin-4-yl)phenyl)carbamate |
| | | | LCMS-ESI (POS.) m/z: 385.1[M + H]⁺ |
| 430 | 30.9 | propionyl chloride | 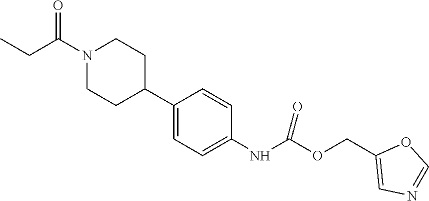 [SMILES: CCC(=O)N1CCC(c2ccc(NC(=O)OCc3cnco3)cc2)CC1] |
| | | | Compound 430: oxazol-5-ylmethyl (4-(1- |
| | | | propionylpiperidin-4-yl)phenyl)carbamate |
| | | | LCMS-ESI (POS.) m/z: 358.1[M + H]⁺ |
| 431 | 30.9 | cyclopropane- carbonyl chloride | 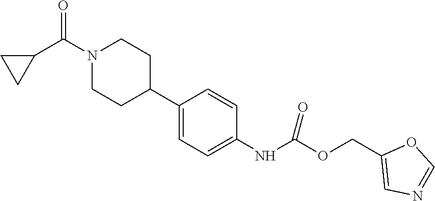 [SMILES: O=C(Nc1ccc(C2CCN(C(=O)C3CC3)CC2)cc1)OCc1cnco1] |
| | | | Compound 431: oxazol-5-ylmethyl (4-(1- |
| | | | (cyclopropanecarbonyl)piperidin-4- |
| | | | yl)phenyl)carbamate |
| | | | LCMS-ESI (POS.) m/z: 370.1[M + H]⁺ |
| 432 | 30.9 | dimethyl- sulfamoyl chloride | 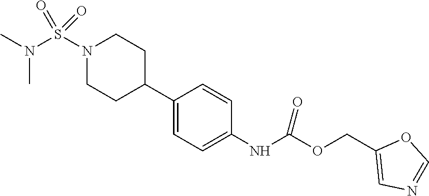 [SMILES: CN(C)S(=O)(=O)N1CCC(c2ccc(NC(=O)OCc3cnco3)cc2)CC1] |
| | | | Compound 432: oxazol-5-ylmethyl (4-(1-(N,N- |
| | | | dimethylsulfamoyl)piperidin-4-yl)phenyl)carbamate |
| | | | LCMS-ESI (POS.) m/z: 409.1[M + H]⁺ |
| 433 | 30.8 | dimethyl- sulfamoyl chloride | 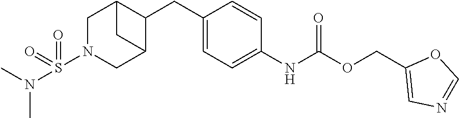 [SMILES: CN(C)S(=O)(=O)N1CC2CC(C1)C2Cc1ccc(NC(=O)OCc2cnco2)cc1] |
| | | | Compound 433: oxazol-5-ylmethyl (4-((3-(N,N- |
| | | | dimethylsulfamoyl)-3-azabicyclo[3.1.1]heptan-6- |
| | | | yl)methyl)phenyl)carbamate |
| | | | LCMS-ESI (POS.) m/z: 435.1[M + H]⁺ |
| 434 | 30.8 | methane- sulfonyl chloride | 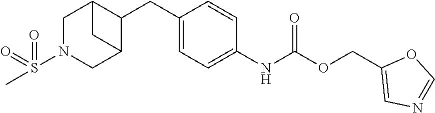 [SMILES: CS(=O)(=O)N1CC2CC(C1)C2Cc1ccc(NC(=O)OCc2cnco2)cc1] |
| | | | Compound 434: oxazol-5-ylmethyl (4-((3- |
| | | | (methylsulfonyl)-3-azabicyclo[3.1.1]heptan-6- |
| | | | yl)methyl)phenyl)carbamate |
| | | | LCMS-ESI (POS.) m/z: 406.1[M + H]⁺ |
| | | | ¹H NMR (400 MHz, Methanol-d₄) δ 8.24 (s, 1H), 7.35 (d, |
| | | | J = 8.0 Hz, 2H), 7.25 (s, 1H), 7.16 (d, J = 8.3 Hz, 2H), |
| | | | 5.25 (s, 2H), 3.67 (d, J = 10.4 Hz, 2H), 3.54 (d, J = 10.7, |
| | | | 1.9 Hz, 2H), 2.96 (s, 3H), 2.71 (d, J = 7.7 Hz, 2H), 2.54 (q, |
| | | | J = 7.1 Hz, 1H), 2.51-2.37 (m, 2H), 2.15-2.05 (m, 1H), |
| | | | 1.45 (d, J = 9.6 Hz, 1H). |
| 435 | 30.8 | methyl carbono- chloridate | 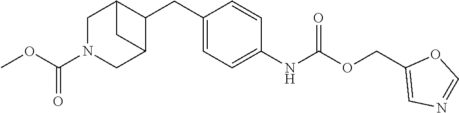 [SMILES: COC(=O)N1CC2CC(C1)C2Cc1ccc(NC(=O)OCc2cnco2)cc1] |
| | | | Compound 435: methyl 6-(4-(((oxazol-5- |
| | | | ylmethoxy)carbonyl)amino)benzyl)-3- |
| | | | azabicyclo[3.1.1]heptane-3-carboxylate |
| | | | LCMS-ESI (POS.) m/z: 386.1[M + H]⁺ |
| 436 | 30.8 | dimethyl- carbamoyl chloride | 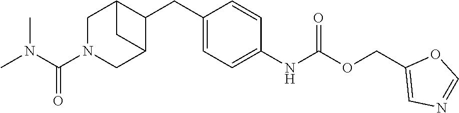 [SMILES: CN(C)C(=O)N1CC2CC(C1)C2Cc1ccc(NC(=O)OCc2cnco2)cc1] |
| | | | Compound 436: oxazol-5-ylmethyl (4-((3- |
| | | | (dimethylcarbamoyl)-3-azabicyclo[3.1.1]heptan-6- |
| | | | yl)methyl)phenyl)carbamate |
| | | | LCMS-ESI (POS.) m/z: 399.1[M + H]⁺ |
| 437 | 30.8 | isobutyryl chloride | 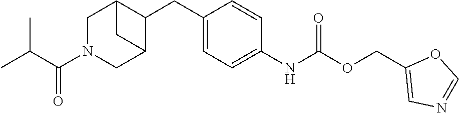 [SMILES: CC(C)C(=O)N1CC2CC(C1)C2Cc1ccc(NC(=O)OCc2cnco2)cc1] |
| | | | Compound 437: oxazol-5-ylmethyl (4-((3-isobutyryl-3- |
| | | | azabicyclo[3.1.1]heptan-6-yl)methyl)phenyl)carbamate |
| | | | LCMS-ESI (POS.) m/z: 398.1[M + H]⁺ |
| 438 | 30.8 | acetic anhydride | 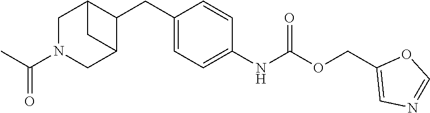 [SMILES: CC(=O)N1CC2CC(C1)C2Cc1ccc(NC(=O)OCc2cnco2)cc1] |
| | | | Compound 438: oxazol-5-ylmethyl (4-((3-acetyl-3- |
| | | | azabicyclo[3.1.1]heptan-6-yl)methyl)phenyl)carbamate |
| | | | LCMS-ESI (POS.) m/z: 370.1[M + H]⁺ |
| 449 | 30.10 | dimethyl- carbamoyl chloride | 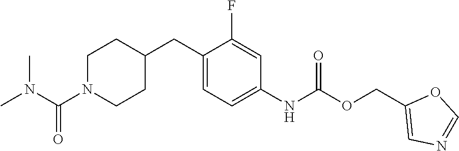 [SMILES: CN(C)C(=O)N1CCC(Cc2ccc(NC(=O)OCc3cnco3)cc2F)CC1] |
| | | | Compound 449: oxazol-5-ylmethyl (4-((1- |
| | | | (dimethylcarbamoyl)piperidin-4-yl)methyl)-3- |
| | | | fluorophenyl)carbamate |
| | | | LCMS-ESI (POS.) m/z: 405.1[M + H]⁺ |
| | | | ¹H NMR (400 MHz, DMSO-d₆) δ 9.96 (s, 1H), 8.42 (s, |
| | | | 1H), 7.36-7.24 (m, 2H), 7.21-7.07 (m, 2H), 5.22 (s, |
| | | | 2H), 3.49 (d, J = 13.1 Hz, 2H), 2.69 (s, 6H), 2.58 (t, J = |
| | | | 12.3 Hz, 2H), 2.47 (d, J = 6.7 Hz, 2H), 1.66-1.57 (m, |
| | | | 1H), 1.51 (d, J = 13.1 Hz, 2H), 1.21-1.01 (m, 2H). |
| 445 | Com- pound 426 | acetic anhydride | 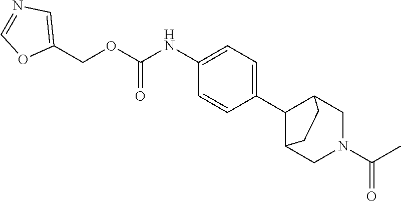 [SMILES: CC(=O)N1CC2CCC(C1)C2c1ccc(NC(=O)OCc2cnco2)cc1] |
| | | | Compound 445: oxazol-5-ylmethyl (4-(3-acetyl-3- |
| | | | azabicyclo[3.2.1]octan-8-yl)phenyl)carbamate |
| | | | LCMS-ESI (POS.) m/z: 370.1 [M + H]⁺ |
| 448 | 30.10 | methane- sulfonyl chloride | 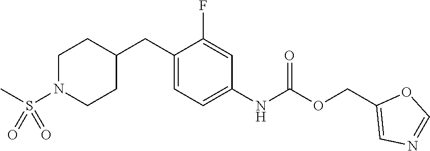 [SMILES: CS(=O)(=O)N1CCC(Cc2ccc(NC(=O)OCc3cnco3)cc2F)CC1] |
| | | | Compound 448: oxazol-5-ylmethyl (3-fluoro-4-((1- |
| | | | (methylsulfonyl)piperidin-4- |
| | | | yl)methyl)phenyl)carbamate |
| | | | LCMS-ESI (POS.) m/z: 412.1[M + H]⁺ |
| | | | ¹H NMR (400 MHz, Chloroform-d) δ 7.92 (s, 1H), 7.28 (s, |
| | | | 1H), 7.20 (s, 1H), 7.18-6.90 (m, 2H), 6.66 (s, 2H), 5.24 |
| | | | (d, J = 10.9 Hz, 2H), 3.77 (d, J = 11.6 Hz, 2H), 2.75 (s, |
| | | | 3H), 2.65-2.50 (m, 4H), 1.73 (d, J = 13.4 Hz, 2H), 1.38 |
| | | | (t, J = 12.5 Hz, 2H). |
| 447 | 30.10 | dimethyl- sulfamoyl chloride | 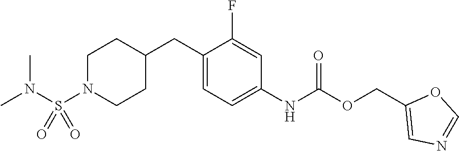 [SMILES: CN(C)S(=O)(=O)N1CCC(Cc2ccc(NC(=O)OCc3cnco3)cc2F)CC1] |
| | | | Compound 447: oxazol-5-ylmethyl (4-((1-(N,N- |
| | | | dimethylsulfamoyl)piperidin-4-yl)methyl)-3- |
| | | | fluorophenyl)carbamate |
| | | | LCMS-ESI (POS.) m/z: 441.1[M + H]⁺ |
| 450 | 30.10 | methyl carbono- chloridate | 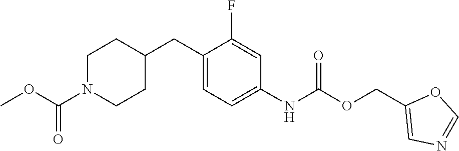 [SMILES: COC(=O)N1CCC(Cc2ccc(NC(=O)OCc3cnco3)cc2F)CC1] |
| | | | Compound 450: methyl 4-(2-fluoro-4-(((oxazol-5- |
| | | | ylmethoxy)carbonyl)amino)benzyl)piperidine-1- |
| | | | carboxylate |
| | | | LCMS-ESI (POS.) m/z: 392.1[M + H]⁺ |
| 451 | 30.10 | acetic anhydride | 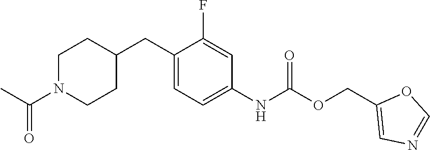 [SMILES: CC(=O)N1CCC(Cc2ccc(NC(=O)OCc3cnco3)cc2F)CC1] |
| | | | Compound 451: oxazol-5-ylmethyl (4-((1- |
| | | | acetylpiperidin-4-yl)methyl)-3-fluorophenyl)carbamate |
| | | | LCMS-ESI (POS.) m/z: 376.1[M + H]⁺ |
| 457 | 30.11 | acetic anhydride | 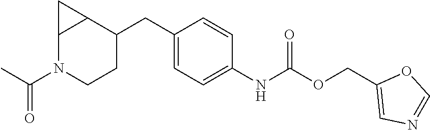 [SMILES: CC(=O)N1CCC(Cc2ccc(NC(=O)OCc3cnco3)cc2)C2CC21] |
| | | | Compound 457: oxazol-5-ylmethyl (4-((2-acetyl-2- |
| | | | azabicyclo[4.1.0]heptan-5-yl)methyl)phenyl)carbamate |
| | | | LCMS-ESI (POS.) m/z: 370.1[M + H]⁺ |
| | | | ¹H NMR (400 MHz, DMSO-d₆) δ 9.72 (s, 1H), 8.41 (s, |
| | | | 1H), 7.40-7.32 (m, 2H), 7.30 (s, 1H), 7.19-7.03 (m, |
| | | | 2H), 5.20 (s, 2H), 4.74-3.88 (m, 1H), 3.71-2.71 (m, |
| | | | 1H), 2.44-2.11 (m, 1H), 2.08-1.84 (m, 4H), 1.75-1.61 |
| | | | (m, 1H), 1.58-1.40 (m, 1H), 1.27-0.95 (m, 3H), 0.89- |
| | | | 0.70 (m, 1H), 0.42-0.17 (m, 1H). |
| 456 | 30.11 | methyl carbono- chloridate | 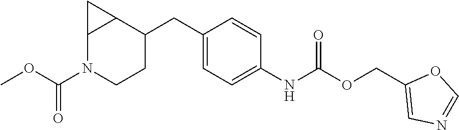 [SMILES: COC(=O)N1CCC(Cc2ccc(NC(=O)OCc3cnco3)cc2)C2CC21] |
| | | | Compound 456: methyl 5-(4-(((oxazol-5- |
| | | | ylmethoxy)carbonyl)amino)benzyl)-2- |
| | | | azabicyclo[4.1.0]heptane-2-carboxylate |
| | | | LCMS-ESI (POS.) m/z: 386.1[M + H]⁺ |
| | | | ¹H NMR (400 MHz, DMSO-d₆) δ 9.71 (s, 1H), 8.41 (s, |
| | | | 1H), 7.39-7.31 (m, 2H), 7.30 (s, 1H), 7.19-7.03 (m, |
| | | | 2H), 5.20 (s, 2H), 3.76-3.51 (m, 4H), 2.85-2.32 (m, |
| | | | 1H), 2.18-1.82 (m, 1H), 1.77-1.37 (m, 2H), 1.25-0.83 |
| | | | (m, 4H), 0.76-0.62 (m, 1H), 0.37-0.16 (m, 1H). |
| 454 | 30.11 | dimethyl- carbamoyl chloride | 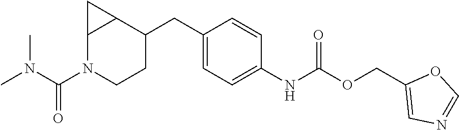 [SMILES: CN(C)C(=O)N1CCC(Cc2ccc(NC(=O)OCc3cnco3)cc2)C2CC21] |
| | | | Compound 454: oxazol-5-ylmethyl (4-((2- |
| | | | (dimethylcarbamoyl)-2-azabicyclo[4.1.0]heptan-5- |
| | | | yl)methyl)phenyl)carbamate |
| | | | LCMS-ESI (POS.) m/z: 399.1[M + H]⁺ |
| | | | ¹H NMR (400 MHz, DMSO-d₆) δ 9.71 (s, 1H), 8.42 (s, |
| | | | 1H), 7.40-7.32 (m, 2H), 7.30 (s, 1H), 7.18-7.02 (m, |
| | | | 2H), 5.20 (s, 2H), 3.31-3.24 (m, 1H), 2.83-2.62 (m, |
| | | | 7H), 2.43-2.30 (m, 1H), 2.15-2.03 (m, 1H), 1.51-1.37 |
| | | | (m, 1H), 1.31-1.18 (m, 1H), 1.14-1.02 (m, 2H), 1.01- |
| | | | 0.89 (m, 1H), 0.88-0.71 (m, 1H), 0.38-0.24 (m, 1H). |
| 453 | 30.11 | methane- sulfonyl chloride | 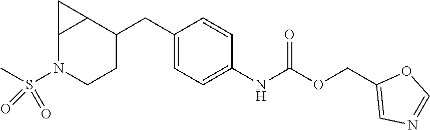 [SMILES: CS(=O)(=O)N1CCC(Cc2ccc(NC(=O)OCc3cnco3)cc2)C2CC21] |
| | | | Compound 453: oxazol-5-ylmethyl (4-((2- |
| | | | (methylsulfonyl)-2-azabicyclo[4.1.0]heptan-5- |
| | | | yl)methyl)phenyl)carbamate |
| | | | LCMS-ESI (POS.) m/z: 406.1[M + H]⁺ |
| | | | ¹H NMR (400 MHz, DMSO-d₆) δ 9.72 (s, 1H), 8.42 (s, |
| | | | 1H), 7.35 (d, J = 7.7 Hz, 2H), 7.30 (s, 1H), 7.19-7.04 (m, |
| | | | 2H), 5.20 (s, 2H), 3.63-3.07 (m, 1H), 2.93-2.55 (m, |
| | | | 5H), 1.91-1.39 (m, 2H), 1.27-0.95 (m, 4H), 0.89-0.64 |
| | | | (m, 1H), 0.60-0.44 (m, 1H). |
| 458 | 320 | morpholine-4- carbonyl chloride | 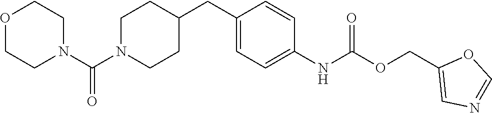 [SMILES: O=C(Nc1ccc(CC2CCN(C(=O)N3CCOCC3)CC2)cc1)OCc1cnco1] |
| | | | Compound 458: oxazol-5-ylmethyl (4-((1-(morpholine- |
| | | | 4-carbonyl)piperidin-4-yl)methyl)phenyl)carbamate |
| | | | LCMS-ESI (POS.) m/z: 429.1[M + H]⁺ |
| 459 | 320 | cyclobutane- sulfonyl chloride | 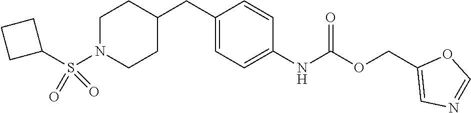 [SMILES: O=C(Nc1ccc(CC2CCN(S(=O)(=O)C3CCC3)CC2)cc1)OCc1cnco1] |
| | | | Compound 459: oxazol-5-ylmethyl (4-((1- |
| | | | (cyclobutylsulfonyl)piperidin-4- |
| | | | yl)methyl)phenyl)carbamate |
| | | | LCMS-ESI (POS.) m/z: 434.1[M + H]⁺ |
| 460 | 320 | cyclopropane- sulfonyl chloride | 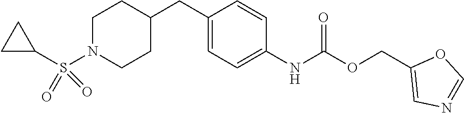 [SMILES: O=C(Nc1ccc(CC2CCN(S(=O)(=O)C3CC3)CC2)cc1)OCc1cnco1] |
| | | | Compound 460: oxazol-5-ylmethyl (4-((1- |
| | | | (cyclopropylsulfonyl)piperidin-4- |
| | | | yl)methyl)phenyl)carbamate |
| | | | LCMS-ESI (POS.) m/z: 420.1[M + H]⁺ |
| 461 | 320 | piperidine-1- carbonyl chloride | 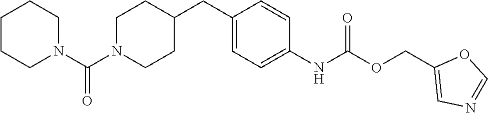 [SMILES: O=C(Nc1ccc(CC2CCN(C(=O)N3CCCCC3)CC2)cc1)OCc1cnco1] |
| | | | Compound 461: oxazol-5-ylmethyl (4-((1-(piperidine-1- |
| | | | carbonyl)piperidin-4-yl)methyl)phenyl)carbamate |
| | | | LCMS-ESI (POS.) m/z: 427.1[M + H]⁺ |
| 462 | 320 | pyrrolidine-1- carbonyl chloride | 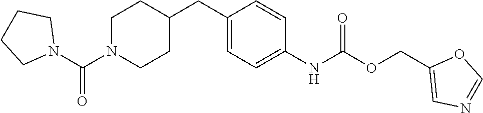 [SMILES: O=C(Nc1ccc(CC2CCN(C(=O)N3CCCC3)CC2)cc1)OCc1cnco1] |
| | | | Compound 462: oxazol-5-ylmethyl (4-((1-(pyrrolidine- |
| | | | 1-carbonyl)piperidin-4-yl)methyl)phenyl)carbamate |
| | | | LCMS-ESI (POS.) m/z: 413.1[M + H]⁺ |
| 464 | 320 | propane-2- sulfonyl chloride | 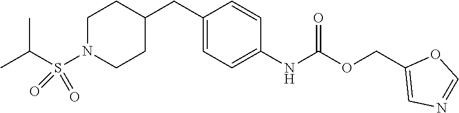 [SMILES: CC(C)S(=O)(=O)N1CCC(Cc2ccc(NC(=O)OCc3cnco3)cc2)CC1] |
| | | | Compound 464: oxazol-5-ylmethyl (4-((1- |
| | | | (isopropylsulfonyl)piperidin-4- |
| | | | yl)methyl)phenyl)carbamate |
| | | | LCMS-ESI (POS.) m/z: 422.1[M + H]⁺ |
| 465 | 320 | azetidine-1- sulfonyl chloride | 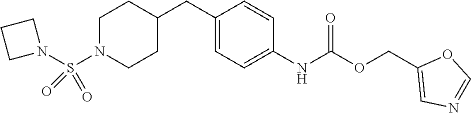 [SMILES: O=C(Nc1ccc(CC2CCN(S(=O)(=O)N3CCC3)CC2)cc1)OCc1cnco1] |
| | | | Compound 465: oxazol-5-ylmethyl (4-((1-(azetidin-1- |
| | | | ylsulfonyl)piperidin-4-yl)methyl)phenyl)carbamate |
| | | | LCMS-ESI (POS.) m/z: 435.1[M + H]⁺ |
| 469 | 320 | cyclobutane- carboxylic acid | 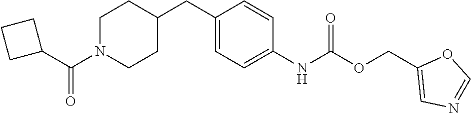 [SMILES: O=C(Nc1ccc(CC2CCN(C(=O)C3CCC3)CC2)cc1)OCc1cnco1] |
| | | | Compound 469: oxazol-5-ylmethyl (4-((1- |
| | | | (cyclobutanecarbonyl)piperidin-4- |
| | | | yl)methyl)phenyl)carbamate |
| | | | LCMS-ESI (POS.) m/z: 398.1[M + H]⁺ |
| 470 | 320 | 3,3- difluorocyclo- butane-1- carboxylic acid | 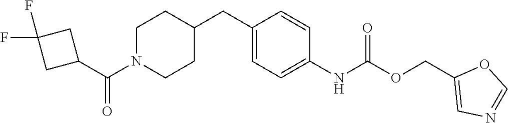 [SMILES: O=C(Nc1ccc(CC2CCN(C(=O)C3CC(F)(F)C3)CC2)cc1)OCc1cnco1] |
| | | | Compound 470: oxazol-5-ylmethyl (4-((1-(3,3- |
| | | | difluorocyclobutane-1-carbonyl)piperidin-4- |
| | | | yl)methyl)phenyl)carbamate |
| | | | LCMS-ESI (POS.) m/z: 334.1[M + H]⁺ |
| 471 | 320 | 2- (bromomethyl) oxetane | 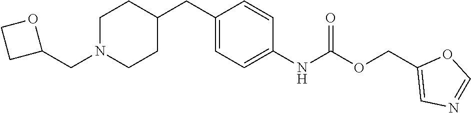 [SMILES: O=C(Nc1ccc(CC2CCN(CC3CCO3)CC2)cc1)OCc1cnco1] |
| | | | Compound 471: oxazol-5-ylmethyl (4-((1-(oxetan-2- |
| | | | ylmethyl)piperidin-4-yl)methyl)phenyl)carbamate |
| | | | LCMS-ESI (POS.) m/z: 386.1[M + H]⁺ |
| 380 | 30.3 | oxetan-3-one | 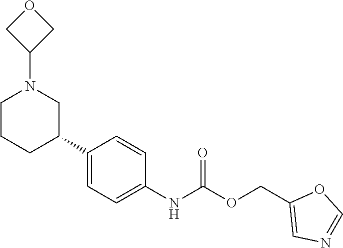 [SMILES: O=C(Nc1ccc([C@@H]2CCCN(C3COC3)C2)cc1)OCc1cnco1] |
| | | | Compound 380: oxazol-5-ylmethyl (S)-(4-(1-(oxetan-3- |
| | | | yl)piperidin-3-yl)phenyl)carbamate |
| | | | LCMS-ESI (POS.) m/z: 358.1[M + H]⁺ |
| | | | ¹H NMR (400 MHz, DMSO-d₆) δ 9.72 (s, 1H), 8.42 (s, |
| | | | 1H), 7.39-7.28 (m, 3H), 7.17 (d, J = 8.2 Hz, 2H), 5.21 (s, |
| | | | 2H), 4.57-4.39 (m, 4H), 3.39 (p, J = 6.5 Hz, 1H), 2.74- |
| | | | 2.63 (m, 3H), 1.77 (s, 3H), 1.72 (d, J = 13.7 Hz, 1H), 1.58 |
| | | | (q, J = 12.4 Hz, 1H), 1.41 (td, J = 12.2, 3.8 Hz, 1H). |
| 441 | 30.7 | oxetan-3-one | 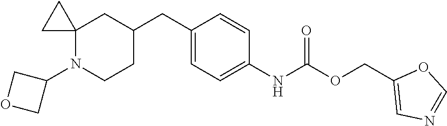 [SMILES: O=C(Nc1ccc(CC2CCN(C3COC3)C3(CC3)C2)cc1)OCc1cnco1] |
| | | | Compound 441: oxazol-5-ylmethyl (4-((4-(oxetan-3-yl)- |
| | | | 4-azaspiro[2.5]octan-7-yl)methyl)phenyl)carbamate |
| | | | LCMS-ESI (POS.) m/z: 398.1[M + H]⁺ |
| | | | ¹H NMR (400 MHz, DMSO-d₆) δ 9.69 (s, 1H), 8.41 (s, |
| | | | 1H), 7.32 (d, J = 8.0 Hz, 2H), 7.29 (s, 1H), 7.08-7.00 (m, |
| | | | 2H), 5.19 (s, 2H), 4.45-4.39 (m, 3H), 4.34 (dd, J = 7.2, |
| | | | 5.6 Hz, 1H), 4.19 (p, J = 7.1 Hz, 1H), 2.69-2.60 (m, 1H), |
| | | | 2.44-2.36 (m, 2H), 1.80-1.69 (m, 1H), 1.64 (td, J = |
| | | | 12.1, 11.6, 1.8 Hz, 1H), 1.28-1.12 (m, 3H), 0.58 (dd, J = |
| | | | 12.4, 3.3 Hz, 1H), 0.55-0.48 (m, 1H), 0.37-0.29 (m, |
| | | | 1H), 0.25-0.18 (m, 1H), 0.10-0.03 (m, 1H). |
| 425 | 426 | oxetan-3-one | 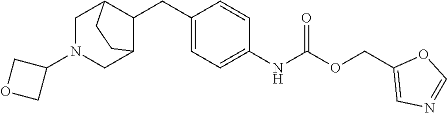 [SMILES: O=C(Nc1ccc(CC2C3CCC2CN(C2COC2)C3)cc1)OCc1cnco1] |
| | | | Compound 425: oxazol-5-ylmethyl (4-((3-(oxetan-3-yl)- |
| | | | 3-azabicyclo[3.2.1]octan-8-yl)methyl)phenyl)carbamate |
| | | | LCMS-ESI (POS.) m/z: 398.1[M + H]⁺ |
| 439 | 30.8 | oxetan-3-one | 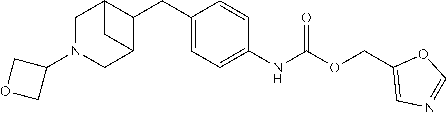 [SMILES: O=C(Nc1ccc(CC2C3CC2CN(C2COC2)C3)cc1)OCc1cnco1] |
| | | | Compound 439: oxazol-5-ylmethyl (4-((3-(oxetan-3-yl)- |
| | | | 3-azabicyclo[3.1.1]heptan-6- |
| | | | yl)methyl)phenyl)carbamate |
| | | | LCMS-ESI (POS.) m/z: 384.1[M + H]⁺ |
| 452 | 30.10 | oxetan-3-one | 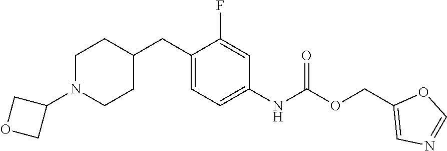 [SMILES: O=C(Nc1ccc(CC2CCN(C3COC3)CC2)c(F)c1)OCc1cnco1] |
| | | | Compound 452: oxazol-5-ylmethyl (3-fluoro-4-((1- |
| | | | (oxetan-3-yl)piperidin-4-yl)methyl)phenyl)carbamate |
| | | | LCMS-ESI (POS.) m/z: 390.1[M + H]⁺ |
| 455 | 30.11 | oxetan-3-one | 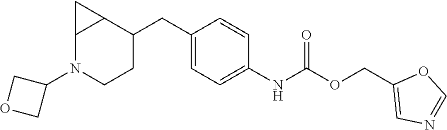 [SMILES: O=C(Nc1ccc(CC2CCN(C3COC3)C3CC23)cc1)OCc1cnco1] |
| | | | Compound 455: oxazol-5-ylmethyl (4-((2-(oxetan-3-yl)- |
| | | | 2-azabicyclo[4.1.0]heptan-5- |
| | | | yl)methyl)phenyl)carbamate |
| | | | LCMS-ESI (POS.) m/z: 384.1[M + H]⁺ |
| | | | ¹H NMR (400 MHz, DMSO-d₆) δ 9.71 (s, 1H), 8.41 (s, |
| | | | 1H), 7.34 (d, J = 8.0 Hz, 2H), 7.30 (s, 1H), 7.14-7.02 (m, |
| | | | 2H), 5.20 (s, 2H), 4.62-4.33 (m, 4H), 3.85-3.76 (m, |
| | | | 1H), 2.47-2.00 (m, 3H), 1.85-0.69 (m, 7H), 0.34-0.24 |
| | | | (m, 1H). |
| 446 | 30.10 | isopropyl carbono- chloridate | 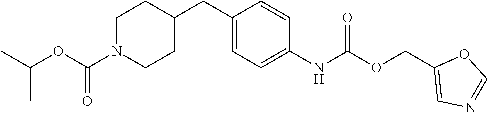 [SMILES: CC(C)OC(=O)N1CCC(Cc2ccc(NC(=O)OCc3cnco3)cc2)CC1] |
| | | | Compound 446: isopropyl 4-(4-(((oxazol-5- |
| | | | ylmethoxy)carbonyl)amino)benzyl)piperidine-1- |
| | | | carboxylate |
| | | | LCMS-ESI (POS.) m/z: 402.1[M + H]⁺ |
| 468 | 30.10 | Triphosgene azetidine | 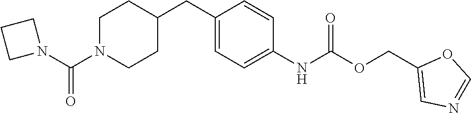 [SMILES: O=C(Nc1ccc(CC2CCN(C(=O)N3CCC3)CC2)cc1)OCc1cnco1] |
| | | | Compound 468: oxazol-5-ylmethyl (4-((1-(azetidine-1- |
| | | | carbonyl)piperidin-4-yl)methyl)phenyl)carbamate. |
| | | | LCMS-ESI (POS.) m/z: 399.1[M + H]⁺ |
| 463 | 30.10 | Triphosgene 3,3-difluoro- azetidine | 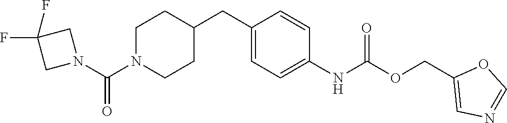 [SMILES: O=C(Nc1ccc(CC2CCN(C(=O)N3CC(F)(F)C3)CC2)cc1)OCc1cnco1] |
| | | | Compound 463: oxazol-5-ylmethyl (4-((1-(3,3- |
| | | | difluoroazetidine-1-carbonyl)piperidin-4- |
| | | | yl)methyl)phenyl)carbamate. |
| | | | LCMS-ESI (POS.) m/z: 435.1[M + H]⁺ |
| 467 | 30.10 | F₃CCH₂OTf | 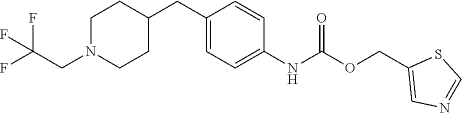 [SMILES: O=C(Nc1ccc(CC2CCN(CC(F)(F)F)CC2)cc1)OCc1cncs1] |
| | | | Compound 467: thiazol-5-ylmethyl (4-((1-(2,2,2- |
| | | | trifluoroethyl)piperidin-4-yl)methyl)phenyl)carbamate. |
| | | | LCMS-ESI (POS.) m/z: 414.1[M + H]⁺ |
| 466 | 30.10 | oxetan-3-one | 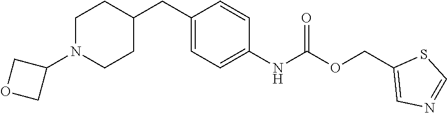 [SMILES: O=C(Nc1ccc(CC2CCN(C3COC3)CC2)cc1)OCc1cncs1] |
| | | | Compound 466: thiazol-5-ylmethyl (4-((1-(oxetan-3- |
| | | | yl)piperidin-4-yl)methyl)phenyl)carbamate. |
| | | | LCMS-ESI (POS.) m/z: 388.1[M + H]⁺ |

### Amide Coupling

### Example M

### Preparation of (4-chlorophenyl)methyl N-(4-{[(2-methylpyridin-4-yl)formamido]methyl}phenyl)carbamate (Compound 173)

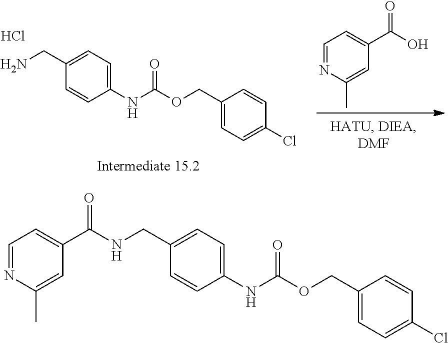

To a solution of (4-chlorophenyl)methyl N-[4-(aminomethyl)phenyl]carbamate hydrochloride (100 mg, 0.31 mmol) and N,N-diisopropylethylamine (0.21 mL, 1.2 mmol) in dimethylformamide (1 mL) was added 2-methylpyridine-4-carboxylic acid (96.4 mg, 0.71 mmol), 1-[Bis(dimethylamino)methylene]-1H-1,2,3-triazolo[4,5-b]pyridinium 3-oxid hexafluorophosphate (151.1 mg, 0.40 mmol), and stirred at rt for 2.5 h, diluted with water, saturated sodium bicarbonate, and extracted with DCM. The combined organic layers were dried over sodium sulfate, concentrated, and purified by silica gel chromatography using a 0-10% MeOH/DCM gradient, concentrated, and re-purified by silica gel chromatography using a 0-100% EtOAc/Hex gradient to yield (4-chlorophenyl)methyl N-(4-{[(2-methylpyridin-4-yl)formamido]methyl}phenyl)carbamate (21.0 mg, 0.05 mmol, 17% yield). LCMS-APCI (POS.) m/z: 410.1 [M+H]⁺. ¹H NMR (400 MHz, DMSO-d₆) δ 9.76 (s, 1H), 9.19 (t, J 6.1 Hz, 1H), 8.57 (d, J=5.1 Hz, 1H), 7.65 (s, 1H), 7.56 (d, J=5.2 Hz, 1H), 7.48-7.44 (m, 4H), 7.41 (d, J 8.3 Hz, 2H), 7.23 (d, J 8.1 Hz, 2H), 5.13 (s, 2H), 4.41 (d, J=5.9 Hz, 2H), 2.53 (s, 3H).

Compounds in the following table were prepared in a similar manner as Compound 173, using the Intermediate and acid as listed.

| Co | Intermediate | | |
|---|---|---|---|
| # | amine | Acid | Structure, Name and Data |
| 21 | 15.2 | 6-methylpyridine-3- carboxylic acid | 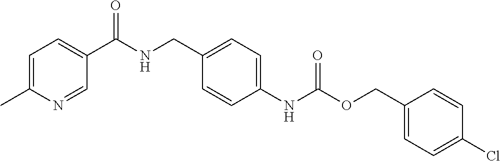 [SMILES: Cc1ccc(C(=O)NCc2ccc(NC(=O)OCc3ccc(Cl)cc3)cc2)cn1] |
| | | | Compound 21: (4-chlorophenyl)methyl N- |
| | | | (4-{[(6-methylpyridin-3- |
| | | | yl)formamido]methyl}phenyl)carbamate. |
| | | | LCMS-ESI (POS.) m/z: 410.0 [M + H]⁺. 1H |
| | | | NMR (300 MHz, DMSO-d6) δ 9.74 (s, 1H), |
| | | | 9.06 (t, J = 5.9 Hz, 1H), 8.90 (m, J = 2.3, 0.8 Hz, |
| | | | 1H), 8.09 (m, J = 8.1, 2.3 Hz, 1H), 7.50- |
| | | | 7.30 (m, 7H), 7.28-7.18 (m, 2H), 5.12 (s, 2H), |
| | | | 4.40 (d, J = 5.9 Hz, 2H),2.50 (s, 3H). |
| 272 | 15.2 | picolinic acid | 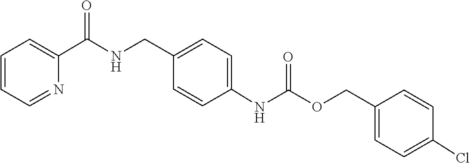 [SMILES: O=C(Nc1ccc(CNC(=O)c2ccccn2)cc1)OCc1ccc(Cl)cc1] |
| | | | Compound 272: (4-chlorophenyl)methyl N- |
| | | | {4-[(pyridin-2- |
| | | | ylformamido)methyl]phenyl}carbamate. |
| | | | LCMS-APCI (POS.) m/z: 396.1 [M + H]⁺. ¹H |
| | | | NMR (400 MHz, DMSO-d₆) δ 9.74 (s, 1H), |
| | | | 9.24 (t, J = 6.2 Hz 1H), 8.64 (d, J = 4.7 Hz, |
| | | | 1H), 8.08-7.94 (m, 2H), 7.60 (t, J = 6.1 Hz, |
| | | | 1H), 7.49-7.42 (m, 4H), 7.39 (d, J = 8.2 Hz, |
| | | | 2H), 7.24 (d, J = 8.1 Hz, 2H), 5.12 (s, 2H), |
| | | | 4.42 (d, J = 6.4 Hz, 2H). |
| 248 | 15.2 | pyridazine-3-carboxylic acid | 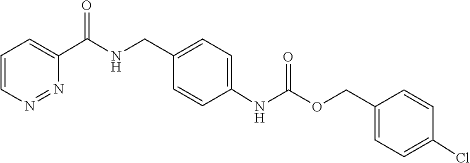 [SMILES: O=C(Nc1ccc(CNC(=O)c2cccnn2)cc1)OCc1ccc(Cl)cc1] |
| | | | Compound 248: (4-chlorophenyl)methyl N- |
| | | | {4-[(pyridazin-3- |
| | | | ylformamido)methyl]phenyl}carbamate. |
| | | | LCMS-APCI (POS.) m/z: 397.1 [M + H]⁺. ¹H |
| | | | NMR (400 MHz, DMSO-d₆) δ 9.80-9.70 (m, |
| | | | 2H), 9.41 (d, J = 4.9 Hz, 1H), 8.21 (d, J = 8.5 Hz, |
| | | | 1H), 7.91 (dd, J = 8.4, 5.1 Hz, 1H), 7.48- |
| | | | 7.42 (m, 4H), 7.40 (d, J = 8.3 Hz, 2H), 7.27 |
| | | | (d, J = 8.3 Hz, 2H), 5.12 (s, 2H), 4.46 (d, J = |
| | | | 6.3 Hz, 2H). |
| 162 | 15.2 | 2-methoxypyrimidine- 5-carboxylic acid | 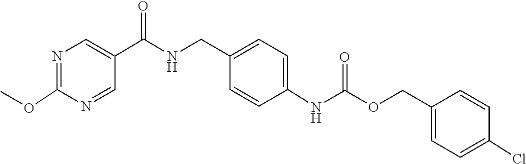 [SMILES: COc1ncc(C(=O)NCc2ccc(NC(=O)OCc3ccc(Cl)cc3)cc2)cn1] |
| | | | Compound 162: (4-chlorophenyl)methyl N- |
| | | | (4-{[(2-methoxypyrimidin-5- |
| | | | yl)formamido]methyl}phenyl)carbamate. |
| | | | LCMS-APCI (POS.) m/z: 427.1 [M + H]+ .. ¹H |
| | | | NMR (400 MHz, DMSO-d₆) δ 9.76 (s, 1H), |
| | | | 9.11 (t, J = 5.9 Hz, 1H), 9.02 (s, 2H), 7.49- |
| | | | 7.44 (m, 4H), 7.41 (d, J = 19.7 Hz, 2H), 7.25 |
| | | | (d, J = 8.2 Hz, 2H), 5.13 (s, 2H), 4.42 (d, J = |
| | | | 5.8 Hz, 2H), 3.98 (s, 3H). |
| 185 | 15.2 | 6- (difluoromethyl) nicotinic acid | 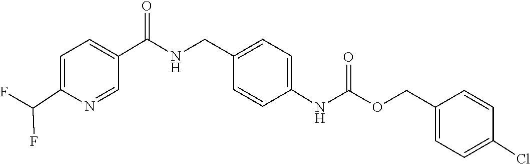 [SMILES: O=C(Nc1ccc(CNC(=O)c2ccc(C(F)F)nc2)cc1)OCc1ccc(Cl)cc1] |
| | | | Compound 185: (4-chlorophenyl)methyl N- |
| | | | [4-({[6-(difluoromethyl)pyridin-3- |
| | | | yl]formamido}methyl)phenyl]carbamate. |
| | | | LCMS-APCI (POS.) m/z: 446.1 [M + H]⁺. ¹H |
| | | | NMR (400 MHz, DMSO-d₆) δ 9.76 (s, 1H), |
| | | | 9.30 (d, J = 5.3 Hz, 1H), 9.11 (s, 1H), 8.40 |
| | | | (d, J = 8.5 Hz, 1H), 7.82 (d, J = 8.5 Hz, 1H), |
| | | | 7.48-7.43 (m, 4H), 7.42 (d, J = 17.5 Hz, 2H), |
| | | | 7.26 (d, J = 8.2 Hz, 2H), 7.03 (t, J = 55.0 Hz, |
| | | | 1H), 5.13 (s, 2H), 4.44 (d, J = 5.7 Hz, 2H). |
| 211 | 15.2 | 2,4-dimethyloxazole-5- carboxylic acid | 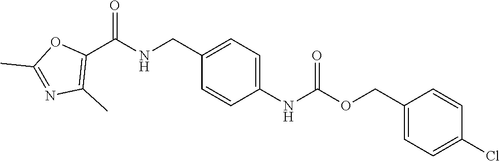 [SMILES: Cc1nc(C)c(C(=O)NCc2ccc(NC(=O)OCc3ccc(Cl)cc3)cc2)o1] |
| | | | Compound 211: (4-chlorophenyl)methyl N- |
| | | | (4-{[(2,4-dimethyl-1,3-oxazol-5- |
| | | | yl)formamido]methyl}phenyl)carbamate. |
| | | | LCMS-APCI (POS.) m/z: 414.2 [M + H]⁺. ¹H |
| | | | NMR (400 MHz, DMSO-d₆) δ 9.75 (s, 1H), |
| | | | 8.76 (t, J = 6.6 Hz, 1H), 7.49-7.42 (m, 4H), |
| | | | 7.39 (d, J = 8.1 Hz, 2H), 7.20 (d, J = 8.1 Hz, |
| | | | 2H), 5.12 (s, 2H), 4.30 (d, J = 6.1 Hz, 2H), |
| | | | 2.41 (s, 3H), 2.30 (s, 3H). |
| 140 | 15.2 | 3,4-dimethylisoxazole- 5-carboxylic acid | 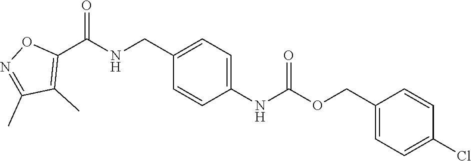 [SMILES: Cc1noc(C(=O)NCc2ccc(NC(=O)OCc3ccc(Cl)cc3)cc2)c1C] |
| | | | Compound 140: (4-chlorophenyl)methyl N- |
| | | | (4-{[(3,4-dimethyl-1,2-oxazol-5- |
| | | | yl)formamido]methyl}phenyl)carbamate. |
| | | | LCMS-APCI (POS.) m/z: 414.1 [M + H]⁺. ¹H |
| | | | NMR (400 MHz, DMSO-d₆) δ 9.79 (s, 1H), |
| | | | 8.45 (t, J = 6.0 Hz, 1H), 7.50-7.45 (m, 4H), |
| | | | 7.43 (d, J = 8.4 Hz, 2H), 7.24 (d, J = 8.1 Hz, |
| | | | 2H), 5.14 (s, 2H), 4.36 (d, J = 5.8 Hz, 2H), |
| | | | 2.49 (s, 3H), 2.29 (s, 3H). |
| 234 | 15.2 | 6-isopropylnicotinic acid | 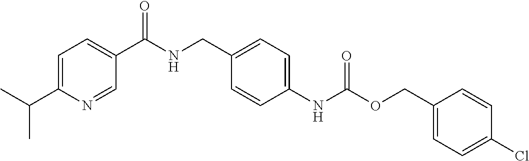 [SMILES: CC(C)c1ccc(C(=O)NCc2ccc(NC(=O)OCc3ccc(Cl)cc3)cc2)cn1] |
| | | | Compound 234: (4-chlorophenyl)methyl N- |
| | | | (4-{[(6-isopropylpyridin-3- |
| | | | yl)formamido]methyl}phenyl)carbamate. |
| | | | LCMS-APCI (POS.) m/z: 438.2 [M + H]⁺. ¹H |
| | | | NMR (400 MHz, DMSO-d₆) δ 9.76 (s, 1H), |
| | | | 9.10 (t, J = 5.6 Hz, 1H), 8.95 (s, 1H), 8.16 |
| | | | (d, J = 8.1 Hz 1H), 7.48-7.44 (m, 4H), 7.41 |
| | | | (d, J = 8.1 Hz, 3H), 7.23 (d, J = 8.1 Hz, 2H), |
| | | | 5.13 (s, 2H), 4.42 (d, J = 5.8 Hz, 2H), 3.08 |
| | | | (sept, J = 7.0 Hz, 1H), 1.24 (d, J = 6.9 Hz, 6H). |
| 294 | 15.2 | 5-methyloxazole-2- carboxylic acid | 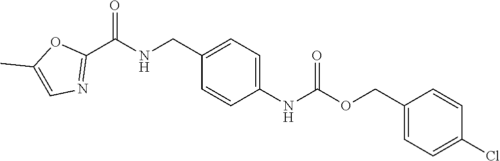 [SMILES: Cc1cnc(C(=O)NCc2ccc(NC(=O)OCc3ccc(Cl)cc3)cc2)o1] |
| | | | Compound 294: (4-chlorophenyl)methyl N- |
| | | | (4-{[(5-methyl-1,3-oxazol-2- |
| | | | yl)formamido]methyl}phenyl)carbamate. |
| | | | LCMS-APCI (POS.) m/z: 400.1 [M + H]+. ¹H |
| | | | NMR (400 MHz, DMSO-d₆) δ 9.75 (s, 1H), |
| | | | 9.27 (t, J = 6.4 Hz, 1H), 7.49-7.42 (m, 4H), |
| | | | 7.39 (d, J = 8.1 Hz, 2H), 7.21 (d, J = 8.5 Hz, |
| | | | 2H), 7.06 (s, 1H), 5.13 (s, 2H), 4.33 (d, J = |
| | | | 6.3 Hz, 2H), 2.37 (s, 3H). |
| 297 | 15.2 | 2-(oxazol-2-yl)acetic acid | 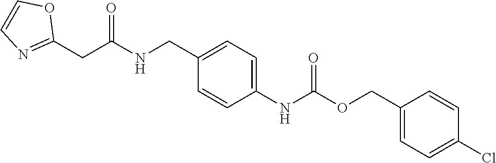 [SMILES: O=C(Cc1ncco1)NCc1ccc(NC(=O)OCc2ccc(Cl)cc2)cc1] |
| | | | Compound 297: (4-chlorophenyl)methyl N- |
| | | | (4-{[2-(1,3-oxazol-2- |
| | | | yl)acetamido]methyl}phenyl)carbamate. |
| | | | LCMS-APCI (POS.) m/z: 400.2 [M + H]⁺. ¹H |
| | | | NMR (400 MHz, DMSO-d₆) δ 9.76 (s, 1H), |
| | | | 8.63 (t, J = 5.1 Hz, 1H), 8.03 (s, 1H), 7.49- |
| | | | 7.43 (m, 4H), 7.41 (d, J = 8.1 Hz, 2H), 7.18 |
| | | | (d, J = 8.2 Hz, 2H), 7.14 (s, 1H), 5.13 (s, 2H), |
| | | | 4.22 (d, J = 5.7 Hz, 2H), 3.73 (s, 2H). |
| 315 | 15.2 | 2-(1-methyl-1H- pyrazol-4-yl)acetic acid | 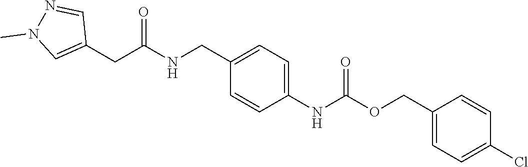 [SMILES: Cn1cc(CC(=O)NCc2ccc(NC(=O)OCc3ccc(Cl)cc3)cc2)cn1] |
| | | | Compound 315: (4-chlorophenyl)methyl N- |
| | | | (4-{[2-(1-methylpyrazol-4- |
| | | | yl)acetamido]methyl}phenyl)carbamate. |
| | | | LCMS-APCI (POS.) m/z: 413.2 [M + H]⁺. ¹H |
| | | | NMR (400 MHz, DMSO-d₆) δ 9.74 (s, 1H), |
| | | | 8.30 (s, 1H), 7.50 (s, 1H), 7.48-7.42 (m, 4H), |
| | | | 7.38 (d, J = 8.3 Hz, 2H), 7.25 (s, 1H), 7.13 |
| | | | (d, J = 8.1 Hz, 2H), 5.13 (s, 2H), 4.17 (d, J = |
| | | | 5.8 Hz, 2H), 3.77 (s, 3H), 3.26 (s, 2H). |
| 295 | 15.2 | 2-(1-methyl-1H- pyrazol-3-yl)acetic acid | 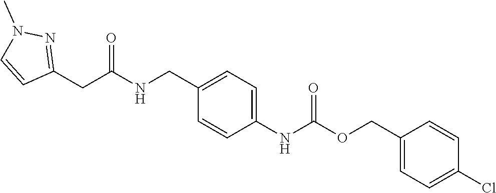 [SMILES: Cn1ccc(CC(=O)NCc2ccc(NC(=O)OCc3ccc(Cl)cc3)cc2)n1] |
| | | | Compound 295: (4-chlorophenyl)methyl N- |
| | | | (4-{[2-(1-methylpyrazol-3- |
| | | | yl)acetamido]methyl}phenyl)carbamate. |
| | | | LCMS-APCI (POS.) m/z: 413.2 [M + H]+. ¹H |
| | | | NMR (400 MHz, DMSO-d₆) δ 9.74 (s, 1H), |
| | | | 8.34 (t, J = 6.0 Hz, 1H), 7.55 (s, 1H), 7.49- |
| | | | 7.42 (m, 4H), 7.38 (d, J = 8.1 Hz, 2H), 7.15 |
| | | | (d, J = 8.0 Hz, 2H), 6.07 (s, 1H), 5.13 (s, 2H), |
| | | | 4.18 (d, J = 5.7 Hz, 2H), 3.76 (s, 3H), 3.39 (s, |
| | | | 2H). |
| 334 | 15.2 | 2-(1H-pyrazol-1- yl)acetic acid | 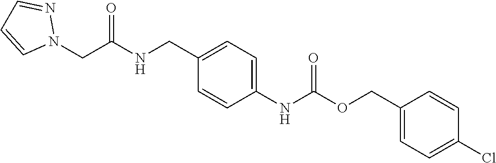 [SMILES: O=C(Cn1cccn1)NCc1ccc(NC(=O)OCc2ccc(Cl)cc2)cc1] |
| | | | Compound 334: (4-chlorophenyl)methyl N- |
| | | | (4-{[2-(pyrazol-1- |
| | | | yl)acetamido]methyl}phenyl)carbamate. |
| | | | LCMS-APCI (POS.) m/z: 399.2 [M + H]⁺. ¹H |
| | | | NMR (400 MHz, DMSO-d₆) δ 9.76 (s, 1H), |
| | | | 8.48 (t, J = 6.0 Hz 1H), 7.71 (s, 1H), 7.49- |
| | | | 7.43 (s, 5H), 7.40 (d, J = 8.1 Hz, 2H), 7.17 |
| | | | (d, J = 8.2 Hz, 2H), 6.25 (s, 1H), 5.13 (s, 2H), |
| | | | 4.83 (s, 2H), 4.22 (d, J = 5.6 Hz, 2H). |
| 209 | 15.2 | 4-methyloxazole-5- carboxylic acid | 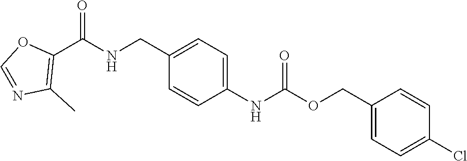 [SMILES: Cc1ncoc1C(=O)NCc1ccc(NC(=O)OCc2ccc(Cl)cc2)cc1] |
| | | | Compound 209: (4-chlorophenyl)methyl N- |
| | | | (4-{[(4-methyl-1,3-oxazol-5- |
| | | | yl)formamido]methyl}phenyl)carbamate. |
| | | | LCMS-APCI (POS.) m/z: 400.1 [M + H]⁺. ¹H |
| | | | NMR (400 MHz, DMSO-d₆) δ 9.74 (s, 1H), |
| | | | 8.90 (t, J = 5.8 Hz 1H), 8.41 (s, 1H), 7.48- |
| | | | 7.42 (m, 4H), 7.39 (d, J = 8.1 Hz, 2H), 7.21 |
| | | | (d, J = 8.1 Hz, 2H), 5.13 (s, 2H), 4.33 (d, J = |
| | | | 6.1 Hz, 2H), 2.36 (s, 3H). |
| 268 | 15.2 | 4-methylisoxazole-5- carboxylic acid | 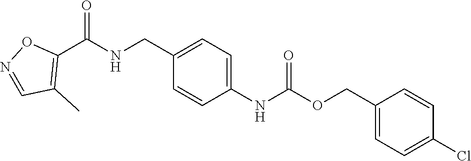 [SMILES: Cc1cnoc1C(=O)NCc1ccc(NC(=O)OCc2ccc(Cl)cc2)cc1] |
| | | | Compound 268: (4-chlorophenyl)methyl N- |
| | | | (4-{[(4-methyl-1,2-oxazol-5- |
| | | | yl)formamido]methyl}phenyl)carbamate. |
| | | | LCMS-APCI (POS.) m/z: 400.1 [M + H]⁺. ¹H |
| | | | NMR (400 MHz, DMSO-d₆) δ 9.75 (s, 1H), |
| | | | 9.30 (t, J = 6.2 Hz 1H), 8.62 (s, 1H), 7.49- |
| | | | 7.43 (m, 4H), 7.40 (d, J = 8.2 Hz, 2H), 7.22 |
| | | | (d, J = 8.3 Hz, 2H), 5.13 (s, 2H), 4.35 (d, J = |
| | | | 6.1 Hz, 2H), 2.22 (s, 3H). |
| 271 | 15.2 | 5-fluoronicotinic acid | 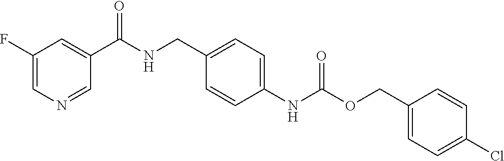 [SMILES: O=C(Nc1ccc(CNC(=O)c2cncc(F)c2)cc1)OCc1ccc(Cl)cc1] |
| | | | Compound 271: (4-chlorophenyl)methyl N- |
| | | | (4-{[(5-fluoropyridin-3- |
| | | | yl)formamido]methyl}phenyl). LCMS- |
| | | | APCI (POS.) m/z: 414.1 [M + H]⁺. ¹H NMR |
| | | | (400 MHz, DMSO-d₆) δ 9.76 (s, 1H), 9.25 |
| | | | (t, J = 5.5 Hz, 1H), 8.92 (s, 1H), 8.74 (s, 1H), |
| | | | 8.11 (d, J = 9.6 Hz, 1H), 7.48-7.44 (m, 4H), |
| | | | 7.42 (d, J = 8.1 Hz, 2H), 7.25 (d, J = 8.2 Hz, |
| | | | 2H), 5.13 (d, J = 2.2 Hz, 2H), 4.43 (d, J = |
| | | | 6.0 Hz, 2H). |
| 205 | 15.2 | 1-methyl-1H-pyrazole- 5-carboxylic acid | 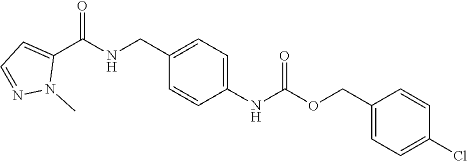 [SMILES: Cn1nccc1C(=O)NCc1ccc(NC(=O)OCc2ccc(Cl)cc2)cc1] |
| | | | Compound 205: (4-chlorophenyl)methyl N- |
| | | | (4-{[(2-methylpyrazol-3- |
| | | | yl)formamido]methyl}phenyl)carbamate. |
| | | | LCMS-APCI (POS.) m/z: 399.1 [M + H]⁺. ¹H |
| | | | NMR (400 MHz, DMSO-d₆) δ 9.75 (s, 1H), |
| | | | 8.94 (t, J = 5.7 Hz 1H), 7.49-7.43 (m, 5H), |
| | | | 7.41 (d, J = 8.1 Hz, 2H), 7.22 (d, J = 8.1 Hz, |
| | | | 2H), 6.88 (s, 1H), 5.13 (s, 2H), 4.36 (d, J = |
| | | | 5.9 Hz, 2H), 4.05 (d, J = 2.5 Hz, 3H). |
| 311 | 15.2 | 2-(oxazol-5-yl)acetic acid | 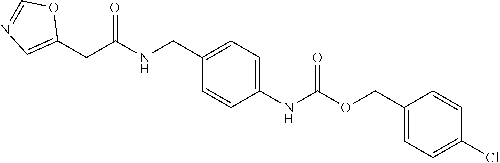 [SMILES: O=C(Cc1cnco1)NCc1ccc(NC(=O)OCc2ccc(Cl)cc2)cc1] |
| | | | Compound 311: (4-chlorophenyl)methyl N- |
| | | | (4-{[2-(1,3-oxazol-5- |
| | | | yl)acetamido]methyl}phenyl)carbamate. |
| | | | LCMS-APCI (POS.) m/z: 400.3 [M + H]⁺. ¹H |
| | | | NMR (400 MHz, DMSO-d₆) δ 9.76 (s, 1H), |
| | | | 8.53 (t, J = 5.6 Hz, 1H), 8.25 (s, 1H), 7.49- |
| | | | 7.43 (m, 4H), 7.40 (d, J = 8.0 Hz, 2H), 7.16 |
| | | | (d, J = 8.1 Hz, 2H), 6.96 (s, 1H), 5.13 (s, 2H), |
| | | | 4.21 (d, J = 5.6 Hz, 2H), 3.64 (s, 2H). |
| 126 | 15.2 | isonicotinic acid | 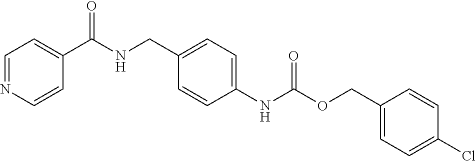 [SMILES: O=C(Nc1ccc(CNC(=O)c2ccncc2)cc1)OCc1ccc(Cl)cc1] |
| | | | Compound 126: (4-chlorophenyl)methyl N- |
| | | | {4-[(pyridin-4- |
| | | | ylformamido)methyl]phenyl}carbamate. |
| | | | LCMS-APCI (POS.) m/z: 396.4 [M + H]⁺. ¹H |
| | | | NMR (400 MHz, DMSO-d₆) δ 9.76 (s, 1H), |
| | | | 9.27 (t, J = 5.9 Hz, 1H), 8.73 (s, 2H), 7.79 (s, |
| | | | 2H), 7.49-7.44 (m, 4H), 7.41 (d, J = 8.4 Hz, |
| | | | 2H), 7.24 (d, J = 8.3 Hz, 2H), 5.13 (s, 2H), |
| | | | 4.42 (d, J = 5.8 Hz, 2H). |
| 321 | 15.2 | 5-methyloxazole-4- carboxylic acid | 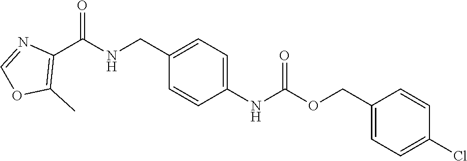 [SMILES: Cc1ocnc1C(=O)NCc1ccc(NC(=O)OCc2ccc(Cl)cc2)cc1] |
| | | | Compound 321: (4-chlorophenyl)methyl N- |
| | | | (4-{[(5-methyl-1,3-oxazol-4- |
| | | | yl)formamido]methyl}phenyl)carbamate. |
| | | | LCMS-APCI (POS.) m/z: 400.3 [M + H]⁺. ¹H |
| | | | NMR (400 MHz, DMSO-d₆) δ 9.73 (s, 1H), |
| | | | 8.62 (t, J = 6.5 Hz, 1H), 8.32 (s, 1H), 7.48- |
| | | | 7.41 (m, 4H), 7.38 (d, J = 8.2 Hz, 2H), 7.21 |
| | | | (d, J = 8.1 Hz, 2H), 5.12 (s, 2H), 4.32 (d, J = |
| | | | 6.0 Hz, 2H), 2.56 (d, J = 2.5 Hz, 3H). |
| 330 | 15.2 | 2-(isoxazol-4-yl)acetic acid | 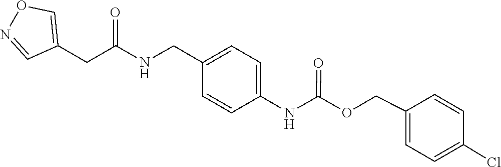 [SMILES: O=C(Cc1cnoc1)NCc1ccc(NC(=O)OCc2ccc(Cl)cc2)cc1] |
| | | | Compound 330: (4-chlorophenyl)methyl N- |
| | | | (4-{[2-(1,2-oxazol-4- |
| | | | yl)acetamido]methyl}phenyl)carbamate. |
| | | | LCMS-APCI (POS.) m/z: 400.3 [M + H]⁺. ¹H |
| | | | NMR (400 MHz, DMSO-d₆) δ 9.75 (s, 1H), |
| | | | 8.75 (s, 1H), 8.53-8.42 (m, 2H), 7.50-7.43 |
| | | | (m, 4H), 7.40 (d, J = 8.1 Hz, 2H), 7.16 (d, J = |
| | | | 8.1 Hz, 2H), 5.13 (s, 2H), 4.20 (d, J = 4.1 Hz, |
| | | | 2H), 3.38 (s, 2H). |
| 225 | 15.2 | 1,3-dimethyl-1H- pyrazole-5-carboxylic acid | 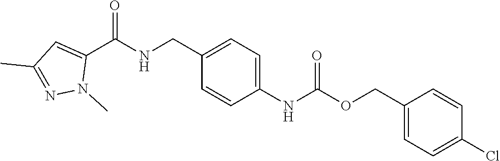 [SMILES: Cc1cc(C(=O)NCc2ccc(NC(=O)OCc3ccc(Cl)cc3)cc2)n(C)n1] |
| | | | Compound 225: (4-chlorophenyl)methyl N- |
| | | | (4-{[(2,5-dimethylpyrazol-3- |
| | | | yl)formamido]methyl}phenyl)carbamate. |
| | | | LCMS-APCI (POS.) m/z: 413.3 [M + H]⁺. ¹H |
| | | | NMR (400 MHz, DMSO-d₆) δ 9.76 (s, 1H), |
| | | | 8.86 (s, 1H), 7.49-7.44 (m, 4H), 7.41 (d, J = |
| | | | 8.1 Hz, 2H), 7.21 (d, J = 8.1 Hz, 2H), 6.64 (s, |
| | | | 1H), 5.14 (s, 2H), 4.34 (d, J = 4.8 Hz, 2H), |
| | | | 3.97 (s, 3H), 2.15 (s, 3H). |
| 45 | 15.2 | 1,4-dimethyl-1H- pyrazole-5-carboxylic acid | 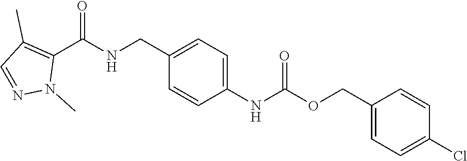 [SMILES: Cc1cnn(C)c1C(=O)NCc1ccc(NC(=O)OCc2ccc(Cl)cc2)cc1] |
| | | | Compound 45: (4-chlorophenyl)methyl N- |
| | | | (4-{[(2,4-dimethylpyrazol-3- |
| | | | yl)formamido]methyl}phenyl)carbamate. |
| | | | LCMS-APCI (POS.) m/z: 413.4 [M + H]⁺. ¹H |
| | | | NMR (400 MHz, DMSO-d₆) δ 9.77 (s, 1H), |
| | | | 8.61 (s, 1H), 7.49-7.44 (m, 4H), 7.42 (d, J = |
| | | | 7.6 Hz, 2H), 7.27-7.21 (m, 3H), 5.13 (s, 2H), |
| | | | 4.38 (d, J = 5.5 Hz, 2H), 3.84 (s, 3H), 2.09 (s, |
| | | | 3H). |
| 167 | 15.2 | 1-ethyl-1H-pyrazole-5- carboxylic acid | 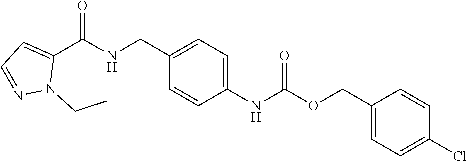 [SMILES: CCn1nccc1C(=O)NCc1ccc(NC(=O)OCc2ccc(Cl)cc2)cc1] |
| | | | Compound 167: (4-chlorophenyl)methyl N- |
| | | | (4-{[(2-ethylpyrazol-3- |
| | | | yl)formamido]methyl}phenyl)carbamate. |
| | | | LCMS-APCI (POS.) m/z: 413.3 [M + H]⁺. ¹H |
| | | | NMR (400 MHz, DMSO-d₆) δ 9.76 (s, 1H), |
| | | | 8.93 (t, J = 7.3 Hz, 1H), 7.49-7.44 (m, 5H), |
| | | | 7.41 (d, J = 8.0 Hz, 2H), 7.22 (d, J = 8.3 Hz, |
| | | | 2H), 6.86 (d, J = 2.7 Hz, 1H), 5.13 (s, 2H), |
| | | | 4.50 (q, J = 7.5 Hz, 2H), 4.36 (d, J = 5.8 Hz, |
| | | | 2H), 1.29 (t, J = 6.8 Hz, 3H). |
| 82 | 15.2 | 6-ethylnicotinic acid | 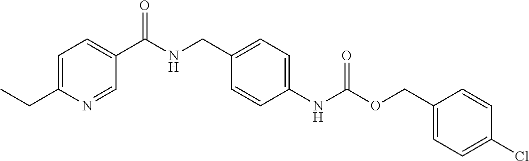 [SMILES: CCc1ccc(C(=O)NCc2ccc(NC(=O)OCc3ccc(Cl)cc3)cc2)cn1] |
| | | | Compound 82: (4-chlorophenyl)methyl N- |
| | | | (4-{[(6-ethylpyridin-3- |
| | | | yl)formamido]methyl}phenyl)carbamate. |
| | | | LCMS-APCI (POS.) m/z: 424.4 [M + H]⁺. ¹H |
| | | | NMR (400 MHz, DMSO-d₆) δ 9.75 (s, 1H), |
| | | | 9.08 (s, 1H), 8.95 (s, 1H), 8.15 (d, J = 8.5 Hz, |
| | | | 1H), 7.49-7.43 (m, 4H), 7.43-7.36 (m, 3H), |
| | | | 7.23 (d, J = 8.3 Hz, 2H), 5.13 (s, 2H), 4.42 (s, |
| | | | 2H), 2.81 (q, J = 8.0 Hz, 2H), 1.28-1.20 (m, |
| | | | 3H). |
| 195 | 15.2 | 5-methoxy-6- methylnicotinic acid | 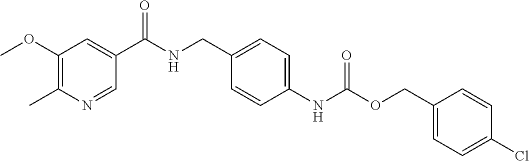 [SMILES: COc1cc(C(=O)NCc2ccc(NC(=O)OCc3ccc(Cl)cc3)cc2)cnc1C] |
| | | | Compound 195: (4-chlorophenyl)methyl N- |
| | | | (4-{[(5-methoxy-6-methylpyridin-3- |
| | | | yl)formamido]methyl}phenyl)carbamate. |
| | | | LCMS-ESI (POS.) m/z: 440.1 [M + H]⁺. ¹H |
| | | | NMR (400 MHz, DMSO-d₆) δ 9.75 (s, 1H), |
| | | | 9.09 (t, J = 5.9 Hz, 1H), 8.52 (d, J = 1.8 Hz, |
| | | | 1H), 7.73 (d, J = 1.7 Hz, 1H), 7.48-7.43 (m, |
| | | | 4H), 7.41 (d, J = 8.6 Hz, 2H), 7.24 (d, J = |
| | | | 8.6 Hz, 2H), 5.13 (s, 2H), 4.43 (d, J = 5.8 Hz, |
| | | | 2H), 3.87 (s, 3H), 2.40 (s, 3H). |
| 301 | 15.2 | 1-isopropyl-1H- pyrazole-5-carboxylic acid | 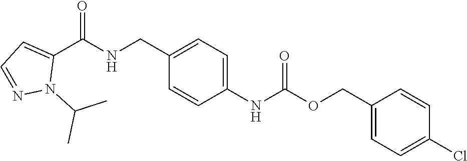 [SMILES: CC(C)n1nccc1C(=O)NCc1ccc(NC(=O)OCc2ccc(Cl)cc2)cc1] |
| | | | Compound 301: (4-chlorophenyl)methyl N- |
| | | | (4-{[(2-isopropylpyrazol-3- |
| | | | yl)formamido]methyl}phenyl)carbamate. |
| | | | LCMS-ESI (POS.) m/z: 427.1 [M + H]⁺. ¹H |
| | | | NMR (400 MHz, DMSO-d₆) δ 9.75 (s, 1H), |
| | | | 8.76 (t, J = 6.0 Hz, 1H), 8.00 (s, 1H), 7.58 (s, |
| | | | 1H), 7.48-7.42 (m, 4H), 7.40 (d, J = 8.3 Hz, |
| | | | 2H), 7.21 (d, J = 8.6 Hz, 2H), 5.24 (p, J = 6.7 Hz, |
| | | | 1H), 5.13 (s, 2H), 4.34 (d, J = 6.0 Hz, 2H), |
| | | | 1.41 (s, 3H), 1.39 (s, 3H). |
| 343 | 15.2 | 4-cyclopropyloxazole- 5-carboxylic acid | 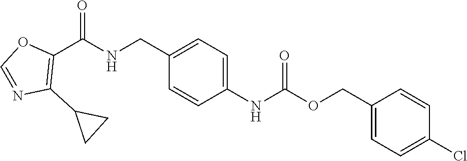 [SMILES: O=C(Nc1ccc(CNC(=O)c2ocnc2C2CC2)cc1)OCc1ccc(Cl)cc1] |
| | | | Compound 343: (4-chlorophenyl)methyl N- |
| | | | (4-{[(4-cyclopropyl-1,3-oxazol-5- |
| | | | yl)formamido]methyl}phenyl)carbamate. |
| | | | LCMS-APCI (POS.) m/z: 426.3 [M + H]⁺. ¹H |
| | | | NMR (400 MHz, DMSO-d₆) δ 9.74 (s, 1H), |
| | | | 8.86 (t, J = 6.2 Hz, 1H), 8.34 (s, 1H), 7.48- |
| | | | 7.42 (m, 4H), 7.40 (d, J = 8.3 Hz, 2H), 7.22 |
| | | | (d, J = 8.6 Hz, 2H), 5.13 (s, 2H), 4.34 (d, J = |
| | | | 6.1 Hz, 2H), 2.62 (td, J = 8.3, 4.3 Hz, 1H), 0.98- |
| | | | 0.92 (m, 2H), 0.86-0.80 (m, 2H). |
| 345 | 15.2 | 3-(difluoromethyl)-1- methyl-1H-pyrazole-5- carboxylic acid | 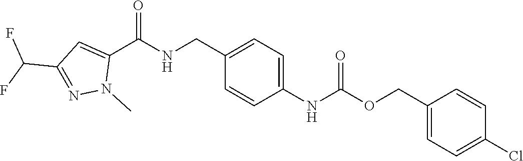 [SMILES: Cn1nc(C(F)F)cc1C(=O)NCc1ccc(NC(=O)OCc2ccc(Cl)cc2)cc1] |
| | | | Compound 345: (4-chlorophenyl)methyl N- |
| | | | [4-({[5-(difluoromethyl)-2-methylpyrazol- |
| | | | 3-yl]formamido}methyl)phenyl]carbamate. |
| | | | LCMS-APCI (POS.) m/z: 449.3 [M + H]⁺. ¹H |
| | | | NMR (400 MHz, DMSO-d₆) δ 9.76 (s, 1H), |
| | | | 9.10 (t, J = 6.0 Hz, 1H), 7.48-7.44 (m, 4H), |
| | | | 7.42 (d, J = 8.5 Hz, 2H), 7.23 (d, J = 8.6 Hz, |
| | | | 2H), 7.15 (t, J = 1.0 Hz, 1H), 7.01 (t, J = 54.6 Hz, |
| | | | 1H), 5.13 (s, 2H), 4.37 (d, J = 5.9 Hz, 2H), |
| | | | 4.09 (s, 3H). |
| 158 | 15.2 | 6-methoxynicotinic acid | 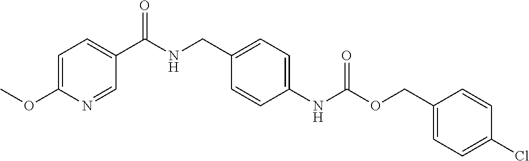 [SMILES: COc1ccc(C(=O)NCc2ccc(NC(=O)OCc3ccc(Cl)cc3)cc2)cn1] |
| | | | Compound 158: (4-chlorophenyl)methyl N- |
| | | | (4-{[(6-methoxypyridin-3- |
| | | | yl)formamido]methyl}phenyl)carbamate. |
| | | | LCMS-APCI (POS.) m/z: 426.3 [M + H]⁺. ¹H |
| | | | NMR (400 MHz, DMSO-d₆) δ 9.76 (s, 1H), |
| | | | 8.98 (t, J = 6.0 Hz, 1H), 8.69 (s, 1H), 8.15 |
| | | | (d, J = 8.6 Hz, 1H), 7.49-7.44 (m, 4H), 7.41 |
| | | | (d, J = 8.1 Hz, 2H), 7.23 (d, J = 8.1 Hz, 2H), |
| | | | 6.89 (d, J = 8.8 Hz, 1H), 5.13 (s, 2H), 4.40 |
| | | | (d, J = 5.8 Hz, 2H), 3.90 (s, 3H). |
| 245 | 15.2 | 2-methoxy-6- methylnicotinic acid | 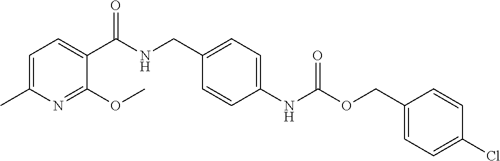 [SMILES: COc1nc(C)ccc1C(=O)NCc1ccc(NC(=O)OCc2ccc(Cl)cc2)cc1] |
| | | | Compound 245: (4-chlorophenyl)methyl N- |
| | | | (4-{[(2-methoxy-6-methylpyridin-3- |
| | | | yl)formamido]methyl}phenyl)carbamate. |
| | | | LCMS-APCI (POS.) m/z: 440.2 [M + H]⁺. ¹H |
| | | | NMR (400 MHz, DMSO-d₆) δ 9.75 (s, 1H), |
| | | | 8.63 (t, J = 8.1 Hz 1H), 8.06 (d, J = 7.6 Hz, |
| | | | 1H), 7.49-7.43 (m, 4H), 7.40 (d, J = 8.2 Hz, |
| | | | 2H), 7.23 (d, J = 8.1 Hz, 2H), 6.97 (d, J = 7.6 Hz, |
| | | | 1H), 5.13 (s, 2H), 4.42 (d, J = 6.0 Hz, 2H), |
| | | | 3.95 (s, 3H), 2.44 (s, 3H). |
| 214 | 15.2 | 5-methylpyridine-2- carboxylic acid | 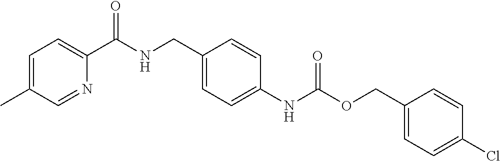 [SMILES: Cc1ccc(C(=O)NCc2ccc(NC(=O)OCc3ccc(Cl)cc3)cc2)nc1] |
| | | | Compound 214: (4-chlorophenyl)methyl N- |
| | | | (4-{[(5-methylpyridin-2- |
| | | | yl)formamido]methyl}phenyl)carbamate. |
| | | | LCMS-APCI (POS.) m/z: 410.1 [M + H]⁺. ¹H |
| | | | NMR (400 MHz, DMSO-d₆) δ 9.74 (s, 1H), |
| | | | 9.16 (t, J = 6.5 Hz, 1H), 8.48 (s, 1H), 7.94 (d, |
| | | | J = 8.0 Hz, 1H), 7.80 (d, J = 7.9 Hz, 1H), 7.50- |
| | | | 7.43 (m, 4H), 7.39 (d, J = 8.1 Hz, 2H), 7.24 |
| | | | (d, J = 8.2 Hz, 2H), 5.13 (s, 2H), 4.41 (d, J = |
| | | | 6.4 Hz, 2H), 2.38 (s, 3H). |
| 255 | 15.2 | 1-methylpyrazole-3- carboxylic acid | 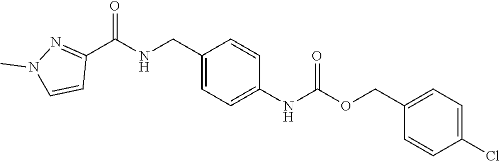 [SMILES: Cn1ccc(C(=O)NCc2ccc(NC(=O)OCc3ccc(Cl)cc3)cc2)n1] |
| | | | Compound 255: (4-chlorophenyl)methyl N- |
| | | | (4-{[(1-methylpyrazol-3- |
| | | | yl)formamido]methyl}phenyl)carbamate. |
| | | | LCMS-APCI (POS.) m/z: 399.1 [M + H]⁺. ¹H |
| | | | NMR (400 MHz, DMSO-d₆) δ 9.73 (s, 1H), |
| | | | 8.54 (t, J = 6.3 Hz, 1H), 7.77 (d, J = 2.1 Hz, |
| | | | 1H), 7.48-7.44 (m, 4H), 7.38 (d, J = 8.2 Hz, |
| | | | 2H), 7.21 (d, J = 8.3 Hz, 2H), 6.61 (d, J = 2.1 Hz, |
| | | | 1H), 5.13 (s, 2H), 4.33 (d, J = 6.3 Hz, 2H), |
| | | | 3.89 (s, 3H). |
| 207 | 15.2 | 3-fluoropyridine-2- carboxylic acid | 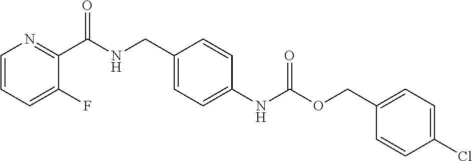 [SMILES: O=C(Nc1ccc(CNC(=O)c2ncccc2F)cc1)OCc1ccc(Cl)cc1] |
| | | | Compound 207: (4-chlorophenyl)methyl N- |
| | | | (4-{[(3-fluoropyridin-2- |
| | | | yl)formamido]methyl}phenyl)carbamate. |
| | | | LCMS-APCI (POS.) m/z: 413.9 [M + H]⁺. ¹H |
| | | | NMR (400 MHz, DMSO-d₆) δ 9.76 (s, 1H), |
| | | | 9.15 (t, J = 6.3 Hz, 1H), 8.48 (d, J = 4.5 Hz, |
| | | | 1H), 7.87 (t, J = 9.7 Hz, 1H), 7.72-7.62 (m, |
| | | | 1H), 7.49-7.38 (m, 6H), 7.25 (d, J = 8.2 Hz, |
| | | | 2H), 5.14 (s, 2H), 4.40 (d, J = 6.3 Hz, 2H). |
| 305 | 15.2 | 5-fluoropyridine-2- carboxylic acid | 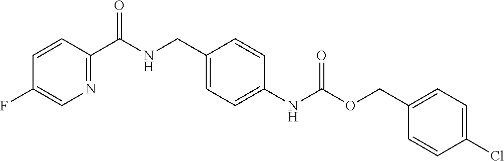 [SMILES: O=C(Nc1ccc(CNC(=O)c2ccc(F)cn2)cc1)OCc1ccc(Cl)cc1] |
| | | | Compound 305: (4-chlorophenyl)methyl N- |
| | | | (4-{[(5-fluoropyridin-2- |
| | | | yl)formamido]methyl}phenyl)carbamate. |
| | | | LCMS-APCI (POS.) m/z: 414.0 [M + H]⁺. ¹H |
| | | | NMR (400 MHz, DMSO-d₆) δ 9.74 (s, 1H), |
| | | | 9.23 (t, J = 6.5 Hz, 1H), 8.68-8.62 (m, 1H), |
| | | | 8.12 (dd, J = 8.8, 4.7 Hz, 1H), 7.91 (td, J = 8.6, |
| | | | 4.6 Hz, 1H), 7.46 (s, 4H), 7.39 (d, J = 8.2 Hz, |
| | | | 2H), 7.24 (d, J = 8.2 Hz, 2H), 5.13 (s, 2H), 4.41 |
| | | | (d, J = 6.3 Hz, 2H). |
| 109 | 15.2 | 2-methylpyrimidine-5- carboxylic acid | 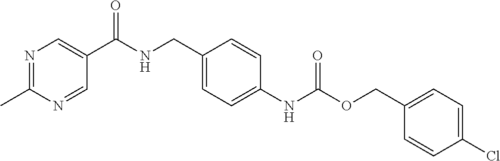 [SMILES: Cc1ncc(C(=O)NCc2ccc(NC(=O)OCc3ccc(Cl)cc3)cc2)cn1] |
| | | | Compound 109: (4-chlorophenyl)methyl N- |
| | | | (4-{[(2-methylpyrimidin-5- |
| | | | yl)formamido]methyl}phenyl)carbamate. |
| | | | LCMS-ES (POS.) m/z: 412.0 [M + H]⁺. ¹H |
| | | | NMR (400 MHz, DMSO-d₆) δ 9.77 (s, 1H), |
| | | | 9.27-9.22 (m, 1H), 9.09 (s, 2H), 7.51-7.38 |
| | | | (m, 6H), 7.26 (d, J = 8.2 Hz, 2H), 5.14 (s, 2H), |
| | | | 4.44 (d, J = 5.9 Hz, 2H), 2.68 (s, 3H). |
| 193 | 15.2 | 1-methylpyrazole-4- carboxylic acid | 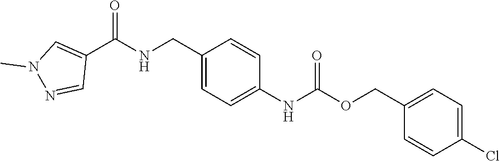 [SMILES: Cn1cc(C(=O)NCc2ccc(NC(=O)OCc3ccc(Cl)cc3)cc2)cn1] |
| | | | Compound 193: (4-chlorophenyl)methyl N- |
| | | | (4-{[(1-methylpyrazol-4- |
| | | | yl)formamido]methyl}phenyl)carbamate. |
| | | | LCMS-ES (POS.) m/z: 398.9 [M + H]⁺. ¹H |
| | | | NMR (400 MHz, DMSO-d₆) δ 9.74 (s, 1H), |
| | | | 8.51 (t, J = 6.2 Hz, 1H), 8.13 (s, 1H), 7.85 (s, |
| | | | 1H), 7.49-7.37 (m, 6H), 7.20 (d, J = 8.1 Hz, |
| | | | 2H), 5.13 (s, 2H), 4.34 (d, J = 5.9 Hz, 2H), 3.85 |
| | | | (s, 3H). |
| 136 | 15.2 | 5-methylpyrazine-2- carboxylic acid | 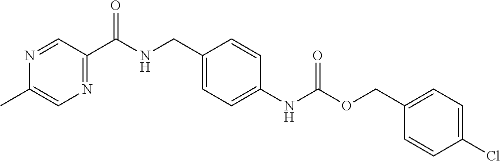 [SMILES: Cc1cnc(C(=O)NCc2ccc(NC(=O)OCc3ccc(Cl)cc3)cc2)cn1] |
| | | | Compound 136: (4-chlorophenyl)methyl N- |
| | | | (4-{[(5-methylpyrazin-2- |
| | | | yl)formamido]methyl}phenyl)carbamate. |
| | | | LCMS-ES (NEG.) m/z: 409.0 [M − H]⁻. ¹H |
| | | | NMR (400 MHz, DMSO-d₆) δ 9.74 (s, 1H), |
| | | | 9.33 (t, J = 6.6 Hz, 1H), 9.05 (s, 1H), 8.62 (s, |
| | | | 1H), 7.48-7.43 (m, 4H), 7.39 (d, J = 8.2 Hz, |
| | | | 2H), 7.24 (d, J = 8.1 Hz, 2H), 5.13 (s, 2H), 4.42 |
| | | | (d, J = 6.3 Hz, 2H), 2.60 (s, 3H). |
| 254 | 15.2 | 1-methylpyrrole-3- carboxylic acid | 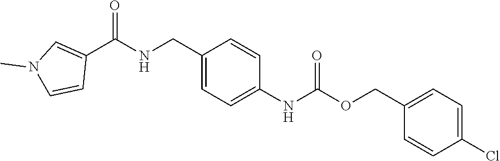 [SMILES: Cn1ccc(C(=O)NCc2ccc(NC(=O)OCc3ccc(Cl)cc3)cc2)c1] |
| | | | Compound 254: (4-chlorophenyl)methyl N- |
| | | | (4-{[(1-methylpyrrol-3- |
| | | | yl)formamido]methyl}phenyl)carbamate. |
| | | | LCMS-ES (POS.) m/z: 398.0 [M + H]⁺. ¹H |
| | | | NMR (400 MHz, DMSO-d₆) δ 9.73 (s, 1H), |
| | | | 8.21 (t, J = 5.2 Hz, 1H), 7.46 (s, 4H), 7.39 (d, |
| | | | J = 8.1 Hz, 2H), 7.26 (s, 1H), 7.18 (d, J = 8.2 Hz, |
| | | | 2H), 6.72-6.67 (m, 1H), 6.49-6.43 (m, |
| | | | 1H), 5.13 (s, 2H), 4.31 (d, J = 5.9 Hz, 2H), 3.62 |
| | | | (s, 3H). |
| 175 | 15.2 | pyrimidine-5- carboxylic acid | 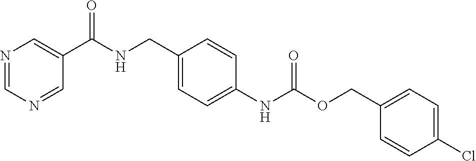 [SMILES: O=C(Nc1ccc(CNC(=O)c2cncnc2)cc1)OCc1ccc(Cl)cc1] |
| | | | Compound 175: (4-chlorophenyl)methyl N- |
| | | | {4-[(pyrimidin-5- |
| | | | ylformamido)methyl]phenyl}carbamate. |
| | | | LCMS-ES (POS.) m/z: 397.0 [M + H]⁺. ¹H |
| | | | NMR (400 MHz, DMSO-d₆) δ 9.77 (s, 1H), |
| | | | 9.37-9.30 (m, 2H), 9.20 (s, 2H), 7.50-7.36 |
| | | | (m, 6H), 7.27 (d, J = 8.2 Hz, 2H), 5.14 (s, 2H), |
| | | | 4.45 (d, J = 5.9 Hz, 2H). |
| 226 | 15.2 | pyrazinoic acid | 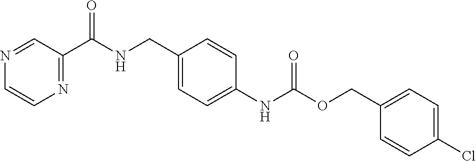 [SMILES: O=C(Nc1ccc(CNC(=O)c2cnccn2)cc1)OCc1ccc(Cl)cc1] |
| | | | Compound 226: (4-chlorophenyl)methyl N- |
| | | | {4-[(pyrazin-2- |
| | | | ylformamido)methyl]phenyl}carbamate. |
| | | | LCMS-APCI (POS.) m/z: 397.1 [M + H]⁺. ¹H |
| | | | NMR (400 MHz, DMSO-d₆) δ 9.75 (s, 1H), |
| | | | 9.41 (t, J = 6.0 Hz, 1H), 9.20 (s, 1H), 8.90- |
| | | | 8.85 (m, 1H), 8.74 (s, 1H), 7.48-7.36 (m, 6H), |
| | | | 7.25 (d, J = 8.2 Hz, 2H), 5.13 (s, 2H), 4.44 (d, |
| | | | J = 6.3 Hz, 2H). |
| 179 | 15.2 | 3-methyloxetane-3- carboxylic acid | 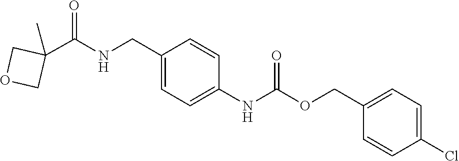 [SMILES: CC1(C(=O)NCc2ccc(NC(=O)OCc3ccc(Cl)cc3)cc2)COC1] |
| | | | Compound 179: (4-chlorophenyl)methyl N- |
| | | | (4-{[(3-methyloxetan-3- |
| | | | yl)formamido]methyl}phenyl)carbamate. |
| | | | LCMS-APCI (POS.) m/z: 389.1 [M + H]⁺. ¹H |
| | | | NMR (400 MHz, DMSO-d₆) δ 9.76 (s, 1H), |
| | | | 8.33-8.28 (m, 1H), 7.49-7.37 (m, 6H), 7.16 |
| | | | (d, J = 8.2 Hz, 2H), 5.14 (s, 2H), 4.73 (d, J = |
| | | | 5.9 Hz, 2H), 4.25 (t, J = 7.2 Hz, 4H), 1.51 (s, |
| | | | 3H). |
| 192 | 15.2 | 2-methylpyridine-3- carboxylic acid | 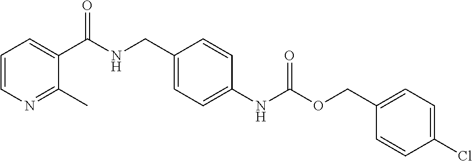 [SMILES: Cc1ncccc1C(=O)NCc1ccc(NC(=O)OCc2ccc(Cl)cc2)cc1] |
| | | | Compound 192: (4-chlorophenyl)methyl N- |
| | | | (4-{[(2-methylpyridin-3- |
| | | | yl)formamido]methyl}phenyl)carbamate. |
| | | | LCMS-APCI (POS.) m/z: 410.1 [M + H]⁺. ¹H |
| | | | NMR (400 MHz, Methanol-d₄) δ 9.37 (s, 1H), |
| | | | 8.52 (s, 1H), 7.93-7.86 (m, 1H), 7.49-7.36 |
| | | | (m, 7H), 7.33 (d, J = 8.2 Hz, 2H), 5.18 (d, J = |
| | | | 2.3 Hz, 2H), 4.52 (s, 2H), 2.62 (s, 3H). Two N—H |
| | | | hydrogens. One hydrogen exchanges with |
| | | | solvent. |
| 165 | 15.2 | 4,6-dimethylpyridine-3- carboxylic acid | 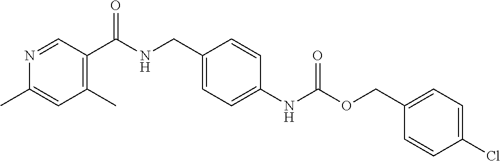 [SMILES: Cc1cc(C)c(C(=O)NCc2ccc(NC(=O)OCc3ccc(Cl)cc3)cc2)cn1] |
| | | | Compound 165: 4-chlorophenyl)methyl N- |
| | | | (4-{[(4,6-dimethylpyridin-3- |
| | | | yl)formamido]methyl}phenyl)carbamate. |
| | | | LCMS-APCI (POS.) m/z: 424.1 [M + H]⁺. ¹H |
| | | | NMR (400 MHz, Methanol-d₄) δ 8.45 (s, 1H), |
| | | | 7.49-7.36 (m, 7H), 7.33 (d, J = 8.2 Hz, 2H), |
| | | | 5.18 (s, 2H), 4.52 (s, 2H), 3.56-3.45 (m, 2H), |
| | | | 2.58 (s, 3H), 2.48 (s, 3H). |
| | | | 1H NMR (400 MHz, DMSO-d6) 8 9.77 (s, 1H), |
| | | | 8.90 (s, 1H), 8.44 (d, J = 7.2 Hz, 1H), 7.50- |
| | | | 7.40 (m, 6H), 7.29-7.20 (m, 3H), 5.14 (s, 2H), |
| | | | 4.42-4.35 (m, 2H), 2.47 (s, 3H), 2.34 (s, 3H). |
| 239 | 15.2 | 6- (trifluoromethyl)pyridine- 3-carboxylic acid | 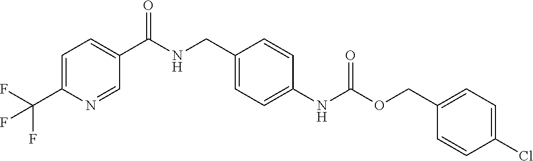 [SMILES: O=C(Nc1ccc(CNC(=O)c2ccc(C(F)(F)F)nc2)cc1)OCc1ccc(Cl)cc1] |
| | | | Compound 239: (4-chlorophenyl)methyl N- |
| | | | [4-({[6-(trifluoromethyl)pyridin-3- |
| | | | yl]formamido}methyl)phenyl]carbamate. |
| | | | LCMS-APCI (POS.) m/z: 464.1 [M + H]⁺. ¹H |
| | | | NMR (400 MHz, DMSO-d₆) δ 9.77 (s, 1H), |
| | | | 9.41-9.36 (m, 1H), 9.18 (s, 1H), 8.49 (d, J = |
| | | | 8.2 Hz, 1H), 8.06 (d, J = 8.5 Hz, 1H), 7.53- |
| | | | 7.36 (m, 6H), 7.27 (d, J = 8.1 Hz, 2H), 5.14 (s, |
| | | | 2H), 4.49-4.43 (m, 2H). |
| 146 | 15.2 | 5-fluoro-6- methylpyridine-3- carboxylic acid | 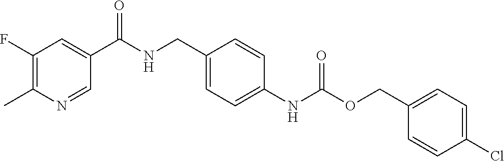 [SMILES: Cc1ncc(C(=O)NCc2ccc(NC(=O)OCc3ccc(Cl)cc3)cc2)cc1F] |
| | | | Compound 146: (4-chlorophenyl)methyl N- |
| | | | (4-{[(5-fluoro-6-methylpyridin-3- |
| | | | yl)formamido]methyl}phenyl)carbamate. |
| | | | LCMS-APCI (POS.) m/z: 428.1 [M + H]+. ¹H |
| | | | NMR (400 MHz, Methanol-d₄) δ 8.75 (s, 1H), |
| | | | 7.96 (d, J = 10.1 Hz, 1H), 7.50-7.35 (m, 6H), |
| | | | 7.31 (d, J = 8.3 Hz, 2H), 5.17 (s, 2H), 4.60 (s, |
| | | | 1H), 4.54 (s, 2H), 2.57 (s, 3H). Two N-H |
| | | | hydrogens. One hydrogen exchanges with |
| | | | solvent. |
| 157 | 15.2 | 2-methyl-1,3-oxazole- 5-carboxylic acid | 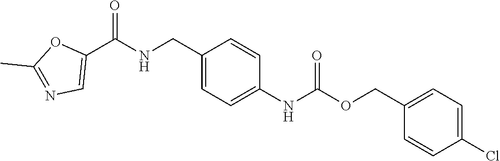 [SMILES: Cc1ncc(C(=O)NCc2ccc(NC(=O)OCc3ccc(Cl)cc3)cc2)o1] |
| | | | Compound 157: (4-chlorophenyl)methyl N- |
| | | | (4-{[(2-methyl-1,3-oxazol-5- |
| | | | yl)formamido]methyl}phenyl)carbamate. |
| | | | LCMS-APCI (POS.) m/z: 400.1 [M + H]⁺. ¹H |
| | | | NMR (400 MHz, DMSO-d₆) δ 9.76 (s, 1H), |
| | | | 8.97 (t, J = 5.7 Hz, 1H), 7.63 (s, 1H), 7.49- |
| | | | 7.37 (m, 6H), 7.21 (d, J = 8.1 Hz, 2H), 5.13 (s, |
| | | | 2H), 4.35 (d, J = 6.0 Hz, 2H), 2.47 (s, 3H). |
| 187 | 15.2 | 1,3-oxazole-5- carboxylic acid | 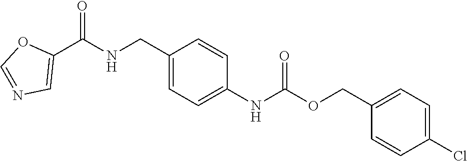 [SMILES: O=C(Nc1ccc(CNC(=O)c2cnco2)cc1)OCc1ccc(Cl)cc1] |
| | | | Compound 187: (4-chlorophenyl)methyl N- |
| | | | {4-[(1,3-oxazol-5- |
| | | | ylformamido)methyl]phenyl}carbamate. |
| | | | LCMS-APCI (POS.) m/z: 386.1 [M + H]⁺. ¹H |
| | | | NMR (400 MHz, DMSO-d₆) δ 9.78 (s, 1H), |
| | | | 9.13 (t, J = 6.1 Hz, 1H), 8.57 (s, 1H), 7.80 (s, |
| | | | 1H), 7.49-7.38 (m, 6H), 7.22 (d, J = 8.1 Hz, |
| | | | 2H), 5.13 (s, 2H), 4.37 (d, J = 6.0 Hz, 2H). |
| 251 | 15.2 | 3-methyl-1,2-oxazole- 4-carboxylic acid | 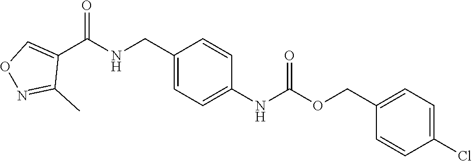 [SMILES: Cc1nocc1C(=O)NCc1ccc(NC(=O)OCc2ccc(Cl)cc2)cc1] |
| | | | Compound 251: (4-chlorophenyl)methyl N- |
| | | | (4-{[(3-methyl-1,2-oxazol-4- |
| | | | yl)formamido]methyl}phenyl)carbamate. |
| | | | LCMS-APCI (POS.) m/z: 400.3 [M + H]⁺. ¹H |
| | | | NMR (400 MHz, DMSO-d₆) δ 9.77 (s, 1H), |
| | | | 9.26 (s, 1H), 8.80 (t, J = 5.9 Hz, 1H), 7.49- |
| | | | 7.39 (m, 6H), 7.23 (d, J = 8.1 Hz, 2H), 5.14 (s, |
| | | | 2H), 4.35 (d, J = 5.6 Hz, 2H), 2.39 (s, 3H). |
| 347 | 15.2 | 4-ethyl-1,3-oxazole-5- carboxylic acid | 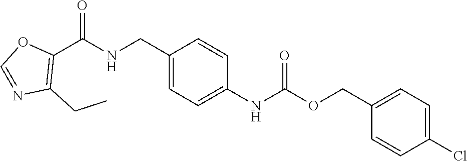 [SMILES: CCc1ncoc1C(=O)NCc1ccc(NC(=O)OCc2ccc(Cl)cc2)cc1] |
| | | | Compound 347: (4-chlorophenyl)methyl N- |
| | | | (4-{[(4-ethyl-1,3-oxazol-5- |
| | | | yl)formamido]methyl}phenyl)carbamate. |
| | | | LCMS-APCI (POS.) m/z: 414.4 [M + H]⁺. ¹H |
| | | | NMR (400 MHz, DMSO-d₆) δ 9.75 (s, 1H), |
| | | | 8.91 (t, J = 6.1 Hz, 1H), 8.42 (s, 1H), 7.51- |
| | | | 7.37 (m, 6H), 7.25-7.18 (m, 2H), 5.13 (s, 2H), |
| | | | 4.34 (d, J = 6.2 Hz, 2H), 2.82 (q, J = 7.5 Hz, |
| | | | 2H), 1.14 (t, J = 7.5 Hz, 3H). |
| 78 | 15.2 | 3-fluoropyridine-4- carboxylic acid | 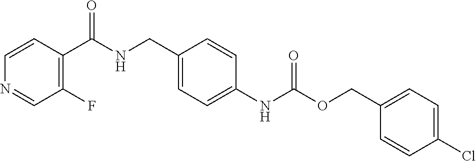 [SMILES: O=C(Nc1ccc(CNC(=O)c2ccncc2F)cc1)OCc1ccc(Cl)cc1] |
| | | | Compound 78: (4-chlorophenyl)methyl N- |
| | | | (4-{[(3-fluoropyridin-4- |
| | | | yl)formamido]methyl}phenyl)carbamate. |
| | | | LCMS-APCI (POS.) m/z: 414.4 [M + H]⁺. ¹H |
| | | | NMR (400 MHz, DMSO-d₆) δ 9.78 (s, 1H), |
| | | | 9.13 (t, J = 5.9 Hz, 1H), 8.72-8.67 (m, 1H), |
| | | | 8.53 (d, 1H), 7.61 (t, J = 5.4 Hz, 1H), 7.51- |
| | | | 7.40 (m, 6H), 7.26 (d, J = 8.3 Hz, 2H), 5.14 (s, |
| | | | 2H), 4.41 (d, J = 5.9 Hz, 2H). |
| 147 | 15.2 | 3,5-difluoropyridine-4- carboxylic acid | 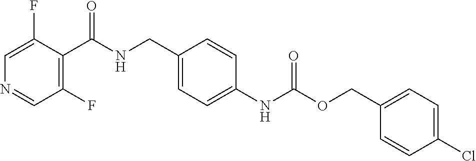 [SMILES: O=C(Nc1ccc(CNC(=O)c2c(F)cncc2F)cc1)OCc1ccc(Cl)cc1] |
| | | | Compound 147: (4-chlorophenyl)methyl N- |
| | | | (4-{[15.2 (3,5-difluoropyridin-4- |
| | | | yl)formamido]methyl}phenyl)carbamate. |
| | | | LCMS-ES (POS.) m/z: 433.0 [M + H]⁺. ¹H |
| | | | NMR (400 MHz, DMSO-d₆) δ 9.80 (s, 1H), |
| | | | 9.43 (t, J = 5.8 Hz, 1H), 8.65 (s, 2H), 7.51- |
| | | | 7.29 (m, 6H), 7.25 (d, J = 8.4 Hz, 2H), 5.15 (s, |
| | | | 2H), 4.44 (d, J = 5.9 Hz, 2H). |
| 166 | 15.2 | 2-(2- methoxyethyl)pyrazole- 3-carboxylic acid | 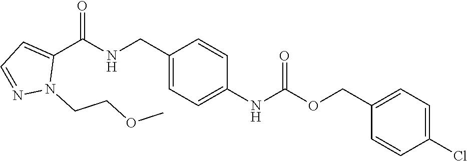 [SMILES: COCCn1nccc1C(=O)NCc1ccc(NC(=O)OCc2ccc(Cl)cc2)cc1] |
| | | | Compound 166: (4-chlorophenyl)methyl N- |
| | | | [4-({[2-(2-methoxyethyl)pyrazol-3- |
| | | | yl]formamido}methyl)phenyl]carbamate. |
| | | | LCMS-APCI (POS.) m/z: 443.1 [M + H]⁺. ¹H |
| | | | NMR (400 MHz, DMSO-d₆) δ 9.77 (s, 1H), |
| | | | 8.96 (s, 1H) 7.52-7.14 (m, 8H), 6.85 (s, 2H), |
| | | | 5.14 (s, 2H), 4.67 (t, J = 5.4 Hz, 2H), 4.36 (d, |
| | | | J = 6.1 Hz, 2H), 3.64 (t, J = 5.6 Hz, 2H), 3.17 |
| | | | (s, 3H). |
| 327 | 15.2 | 2-cyclobutylpyrazole-3- carboxylic acid | 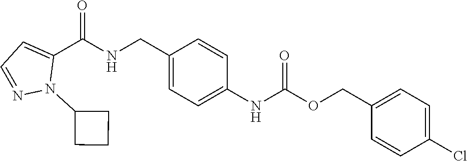 [SMILES: O=C(Nc1ccc(CNC(=O)c2ccnn2C2CCC2)cc1)OCc1ccc(Cl)cc1] |
| | | | Compound 327: (4-chlorophenyl)methyl N- |
| | | | (4-{[(2-cyclobutylpyrazol-3- |
| | | | yl)formamido]methyl}phenyl)carbamate. |
| | | | LCMS-ES (POS.) m/z: 439.1 [M + H]⁺. ¹H |
| | | | NMR (400 MHz, DMSO-d₆) δ 9.77 (s, 1H), |
| | | | 8.94 (t, J = 6.0 Hz, 1H), 7.54 (s, 1H), 7.52- |
| | | | 7.36 (m, 6H), 7.22 (d, J = 8.1 Hz, 2H), 6.84 (s, |
| | | | 1H), 5.72-5.65 (m, 1H), 5.14 (s, 2H), 4.35 (d, |
| | | | J = 6.0 Hz, 2H), 2.32-2.26 (m, 2H), 1.85- |
| | | | 1.66 (m, 4H). |
| 203 | 15.2 | 4-fluoro-2,5- dimethylpyrazole-3- carboxylic acid | 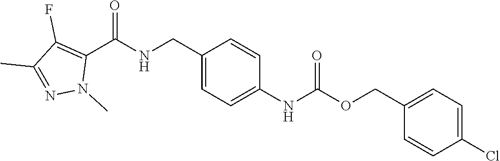 [SMILES: Cc1nn(C)c(C(=O)NCc2ccc(NC(=O)OCc3ccc(Cl)cc3)cc2)c1F] |
| | | | Compound 203: (4-chlorophenyl)methyl N- |
| | | | (4-{[(4-fluoro-2,5-dimethylpyrazol-3- |
| | | | yl)formamido]methyl}phenyl)carbamate. |
| | | | LCMS-ES (POS.) m/z: 431.1 [M + H]⁺. ¹H |
| | | | NMR (400 MHz, DMSO-d₆) δ 9.77 (s, 1H), |
| | | | 8.60 (s, 1H), 7.49-7.21 (m, 8H), 5.14 (s, 2H), |
| | | | 4.38 (d, J = 6.1 Hz, 2H), 3.87 (s, 3H), 2.14 (s, |
| | | | 3H). |
| 208 | 15.2 | 5-ethyl-2- methylpyrazole-3- carboxylic acid | 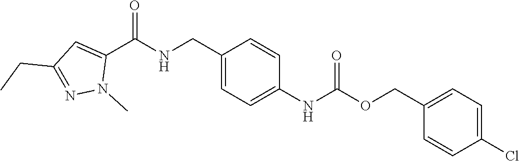 [SMILES: CCc1cc(C(=O)NCc2ccc(NC(=O)OCc3ccc(Cl)cc3)cc2)n(C)n1] |
| | | | Compound 208: (4-chlorophenyl)methyl N- |
| | | | (4-{[(5-ethyl-2-methylpyrazol-3- |
| | | | yl)formamido]methyl}phenyl)carbamate. |
| | | | LCMS-APCI (POS.) m/z: 427.1 [M + H]⁺. ¹H |
| | | | NMR (400 MHz, DMSO-d₆) δ 9.76 (s, 1H), |
| | | | 8.87 (d, J = 6.2 Hz, 1H), 7.50-7.18 (m, 8H), |
| | | | 6.69 (s, 1H), 5.14 (s, 2H), 4.34 (d, J = 5.9 Hz, |
| | | | 2H), 3.98 (s, 3H), 2.57-2.52 (m, 2H), 1.16 (t, |
| | | | J = 7.6 Hz, 3H). |
| | | | ¹H NMR (400 MHz, Methanol-d₄) δ 8.10 (s, |
| | | | 1H), 7.41 (q, J = 8.2 Hz, 5H), 7.28 (d, J = 8.2 Hz, |
| | | | 2H), 6.61 (s, 1H), 5.17 (s, 2H), 4.47 (d, J = |
| | | | 4.9 Hz, 2H), 4.06 (s, 3H), 2.62 (q, J = 7.8 Hz, |
| | | | 2H), 1.25 (t, J = 7.7 Hz, 3H). Two N—H |
| | | | hydrogens. Two hydrogens exchange with |
| | | | solvent. |
| 302 | 15.2 | 2-(2- methylpropyl)pyrazole- 3-carboxylic acid | 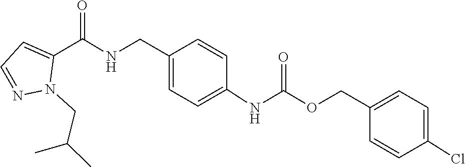 [SMILES: CC(C)Cn1nccc1C(=O)NCc1ccc(NC(=O)OCc2ccc(Cl)cc2)cc1] |
| | | | Compound 302: (4-chlorophenyl)methyl N- |
| | | | [4-({[2-(2-methylpropyl)pyrazol-3- |
| | | | yl]formamido}methyl)phenyl]carbamate. |
| | | | LCMS-ES (POS.) m/z: 441.1 [M+H]+. 1H |
| | | | NMR (400 MHz, DMSO-d₆) δ 9.76 (s, 1H), |
| | | | 8.95 (t, J = 5.8 Hz, 1H), 7.51-7.38 (m, 7H), |
| | | | 7.21 (d, J = 8.0 Hz, 2H), 6.87 (d, J = 2.3 Hz, |
| | | | 1H), 5.14 (s, 2H), 4.39-4.30 (m, 4H), 2.08 |
| | | | (dt, J = 13.9, 6.8 Hz, 1H), 0.78 (d, J = 6.6 Hz, |
| | | | 6H). |
| 138 | 15.2 | 5-methoxy-2- methylpyrazole-3- carboxylic acid | 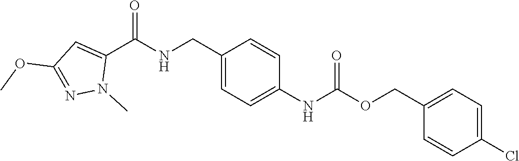 [SMILES: COc1cc(C(=O)NCc2ccc(NC(=O)OCc3ccc(Cl)cc3)cc2)n(C)n1] |
| | | | Compound 138: (4-chlorophenyl)methyl N- |
| | | | (4-{[(5-methoxy-2-methylpyrazol-3- |
| | | | yl)formamido]methyl}phenyl)carbamate. |
| | | | LCMS-ES (POS.) m/z: 429.1 [M + H]⁺. ¹H |
| | | | NMR (400 MHz, DMSO-d₆) δ 9.77 (s, 1H), |
| | | | 8.89 (t, J = 4.7 Hz, 1H), 7.49-7.38 (m, 6H), |
| | | | 7.22 (d, J = 8.2 Hz, 2H), 6.30 (s, 1H), 5.14 (s, |
| | | | 2H), 4.34 (d, J = 5.8 Hz, 2H), 3.91 (s, 3H), 3.76 |
| | | | (s, 3H). |
| 135 | 15.2 | 4-methoxy-2- methylpyrazole-3- carboxylic acid | 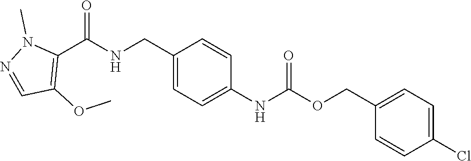 [SMILES: COc1cnn(C)c1C(=O)NCc1ccc(NC(=O)OCc2ccc(Cl)cc2)cc1] |
| | | | Compound 135: (4-chlorophenyl)methyl N- |
| | | | (4-{[(4-methoxy-2-methylpyrazol-3- |
| | | | yl)formamido]methyl}phenyl)carbamate. |
| | | | LCMS-ES (POS.) m/z: 429.0 [M + H]⁺. ¹H |
| | | | NMR (400 MHz, DMSO-d₆) δ 9.76 (s, 1H), |
| | | | 7.86 (t, J = 5.7 Hz, 1H), 7.54-7.34 (m, 7H), |
| | | | 7.23 (d, J = 7.9 Hz, 2H), 5.14 (s, 2H), 4.41 (d, |
| | | | J = 6.0 Hz, 2H), 4.00 (s, 3H), 3.84 (s, 3H). |
| 19 | 15.2 | trimethylpyrazole-3- carboxylic acid | 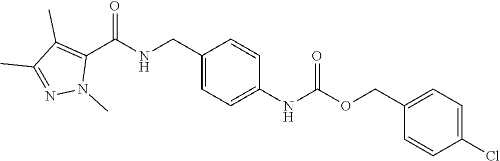 [SMILES: Cc1nn(C)c(C(=O)NCc2ccc(NC(=O)OCc3ccc(Cl)cc3)cc2)c1C] |
| | | | Compound 19: (4-chlorophenyl)methyl N- |
| | | | (4-{[(2,4,5-trimethylpyrazol-3- |
| | | | yl)formamido]methyl}phenyl)carbamate. |
| | | | LCMS-APCI (POS.) m/z: 429.2 [M + H]⁺. ¹H |
| | | | NMR (400 MHz, DMSO-d₆) δ 9.77 (s, 1H), |
| | | | 8.60 (s, 1H), 7.52-7.39 (m, 6H), 7.28-7.21 |
| | | | (m, 2H), 5.14 (d, J = 2.3 Hz, 2H), 4.38 (d, J = |
| | | | 5.9 Hz, 2H), 3.76 (d, J = 2.2 Hz, 3H), 2.07 (d, |
| | | | J = 2.3 Hz, 3H), 2.00 (d, J = 2.2 Hz, 3H). |
| 238 | 15.2 | 2-(oxan-4-yl)pyrazole- 3-carboxylic acid | 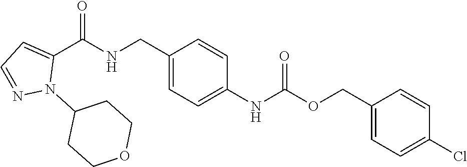 [SMILES: O=C(Nc1ccc(CNC(=O)c2ccnn2C2CCOCC2)cc1)OCc1ccc(Cl)cc1] |
| | | | Compound 238: (4-chlorophenyl)methyl N- |
| | | | [4-({[2-(oxan-4-yl)pyrazol-3- |
| | | | 15.2yl]formamido}methyl)phenyl]carbama |
| | | | te. LCMS-APCI (POS.) m/z: 470.3 [M + H]⁺. |
| | | | ¹H NMR (400 MHz, DMSO-d₆) δ 9.76 (s, 1H), |
| | | | 8.98 (t, J = 5.4 Hz, 1H), 7.57-7.18 (m, 9H), |
| | | | 6.87 (s, 1H), 5.40-5.31 (m, 1H), 5.14 (s, 2H), |
| | | | 4.37 (d, J = 5.9 Hz, 2H), 3.95 (d, J = 11.5 Hz, |
| | | | 2H), 3.42 (t, J = 11.9 Hz, 2H), 2.04 (dd, J = |
| | | | 22.8, 11.9 Hz, 2H), 1.82 (d, J = 12.2 Hz, 2H). |
| 124 | 15.2 | 1-methyl-2-oxo-4- piperidinecarboxylic acid | 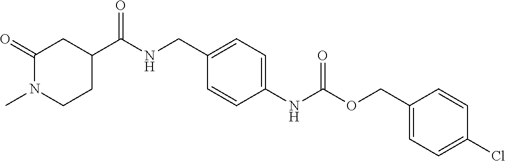 [SMILES: CN1CCC(C(=O)NCc2ccc(NC(=O)OCc3ccc(Cl)cc3)cc2)CC1=O] |
| | | | Compound 124: (4-chlorophenyl)methyl N- |
| | | | (4-{[(1-methyl-2-oxopiperidin-4- |
| | | | yl)formamido]methyl}phenyl)carbamate. |
| | | | Example: LCMS-ES (Pos) m/z: 430 [M + H]⁺. |
| | | | ¹H NMR (400 MHz, DMSO-d₆) δ 9.76 (s, 1H), |
| | | | 8.35 (d, J = 6.4 Hz, 1H), 7.43 (m, 6H), 7.15 (d, |
| | | | J = 8.1 Hz, 2H), 5.14 (s, 2H), 4.20 (m, 2H), |
| | | | 3.27-3.21 (m, 2H), 2.79 (s, 3H), 2.69 (d, J = |
| | | | 9.8 Hz, 1H), 2.31 (d, J = 7.8 Hz, 2H), 1.99- |
| | | | 1.89 (m, 1H), 1.78 (s, 1H). |
| 276 | 15.2 | 4-methyl-1,3-oxazole- 2-carboxylic acid | 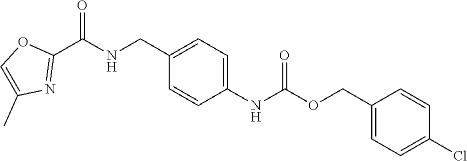 [SMILES: Cc1coc(C(=O)NCc2ccc(NC(=O)OCc3ccc(Cl)cc3)cc2)n1] |
| | | | Compound 276: (4-chlorophenyl)methyl N- |
| | | | (4-{[(4-methyl-1,3-oxazol-2- |
| | | | yl)formamido]methyl}phenyl)carbamate. |
| | | | LCMS-ES (Pos) m/z: 400 [M + H]⁺. ¹H NMR |
| | | | (400 MHz, DMSO-d₆) δ 9.76 (s, 1H), 9.37 (d, |
| | | | J = 6.6 Hz, 1H), 8.01 (s, 1H), 7.46 (d, J = 2.1 Hz, |
| | | | 4H), 7.40 (d, J = 8.1 Hz, 2H), 7.22 (d, J = |
| | | | 8.1 Hz, 2H), 5.13 (s, 2H), 4.33 (d, J = 6.3 Hz, |
| | | | 2H), 2.16 (s, 3H). |
| 277 | 15.2 | 4-carboxy-1- methylpyrrolidin-2-one | 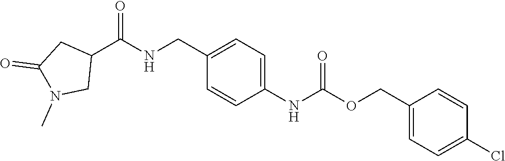 [SMILES: CN1CC(C(=O)NCc2ccc(NC(=O)OCc3ccc(Cl)cc3)cc2)CC1=O] |
| | | | Compound 277: (4-chlorophenyl)methyl N- |
| | | | (4-{[(1-methyl-5-oxopyrrolidin-3- |
| | | | yl)formamido]methyl}phenyl)carbamate. |
| | | | LCMS-ES (Pos) m/z: 416 [M + H]⁺. ¹H NMR |
| | | | (400 MHz, DMSO-d₆) δ 9.76 (s, 1H), 8.47 (s, |
| | | | 1H), 7.50-7.38 (m, 6H), 7.16 (d, J = 8.1 Hz, |
| | | | 2H), 5.14 (s, 2H), 4.21 (d, J = 5.7 Hz, 2H), 3.51 |
| | | | (t, J = 9.3 Hz, 1H), 3.39-3.34 (m, 1H), 3.20- |
| | | | 3.09 (m, 1H), 2.70 (s, 3H), 2.41 (d, J = 8.8 Hz, |
| | | | 2H). |
| 310 | 15.2 | 3-oxetanecarboxylic acid | 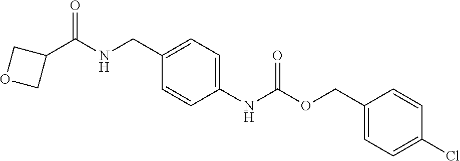 [SMILES: O=C(Nc1ccc(CNC(=O)C2COC2)cc1)OCc1ccc(Cl)cc1] |
| | | | Compound 310: (4- |
| | | | chlorophenyl)methyl N-{4-[(oxetan-3- |
| | | | ylformamido)methyl]phenyl}carbamate. |
| | | | LCMS-ES (Pos) m/z: 375 [M + H]⁺. ¹H NMR |
| | | | (400 MHz, DMSO-d₆) δ 9.75 (s, 1H), 8.32 (s, |
| | | | 1H), 7.43 (d, J = 28.0 Hz, 6H), 7.16 (d, J = |
| | | | 8.3 Hz, 2H), 5.14 (s, 2H), 4.62 (d, J = 7.6 Hz, |
| | | | 3H), 4.22 (d, J = 5.8 Hz, 2H), 3.78 (q, J = 7.4 Hz, |
| | | | 1H), 3.17 (d, J = 5.2 Hz, 1H). |
| 285 | 15.2 | 4-carboxytetrahydro- 2h-pyran | 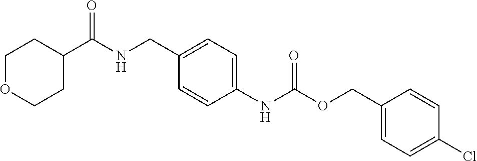 [SMILES: O=C(Nc1ccc(CNC(=O)C2CCOCC2)cc1)OCc1ccc(Cl)cc1] |
| | | | Compound 285: (4-chlorophenyl)methyl N- |
| | | | {4-[(oxan-4- |
| | | | ylformamido)methyl]phenyl}carbamate |
| | | | LCMS-ES (Pos) m/z: 403 [M + H]⁺. ¹H NMR |
| | | | (400 MHz, DMSO-d₆) δ 9.74 (s, 1H), 8.24 (t, |
| | | | J = 6.0 Hz, 1H), 7.49-7.43 (m, 4H), 7.39 (d, |
| | | | J = 8.1 Hz, 2H), 7.14 (d, J = 8.1 Hz, 2H), |
| | | | 5.14 (s, 2H), 4.19 (d, J = 5.9 Hz, 2H), 3.90- |
| | | | 3.81 (m, 2H), 3.32-3.26 (m, 2H), 2.39 (p, J = |
| | | | 7.7 Hz, 1H), 1.64-1.55 (m, 4H). |
| 356 | 15.2 | 6-methyl-3- pyridinesulfonyl chloride | 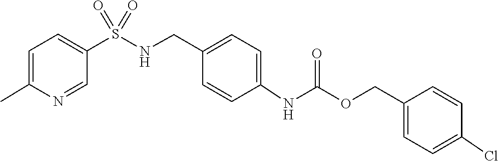 [SMILES: Cc1ccc(S(=O)(=O)NCc2ccc(NC(=O)OCc3ccc(Cl)cc3)cc2)cn1] |
| | | | Example 356: (4-chlorophenyl)methyl N-{4- |
| | | | [(6-methylpyridine-3- |
| | | | sulfonamido)methyl]phenyl}carbamate. |
| | | | LCMS-ES (Pos) m/z: 446 [M + H]⁺. ¹H NMR |
| | | | (400 MHz, DMSO-d₆) δ 9.77 (s, 1H), 8.75 (s, |
| | | | 1H), 8.24 (s, 1H), 8.02-7.94 (m, 1H), 7.46 (s, |
| | | | 2H), 7.42 (d, J = 8.5 Hz, 4H), 7.34 (d, J = 8.1 Hz, |
| | | | 2H), 7.11 (d, J = 8.2 Hz, 1H), 5.14 (s, 2H), |
| | | | 3.95 (d, J = 6.2 Hz, 2H), 2.54 (s, 3H). |
| 76 | 16.2 | 6-ethylnicotinic acid | 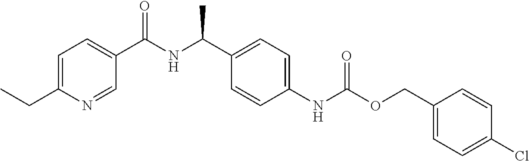 [SMILES: CCc1ccc(C(=O)N[C@@H](C)c2ccc(NC(=O)OCc3ccc(Cl)cc3)cc2)cn1] |
| | | | Compound 76: (4-chlorophenyl)methyl N- |
| | | | {4-[(1S)-1-[(6-ethylpyridin-3- |
| | | | yl)formamido]ethyl]phenyl}carbamate. |
| | | | LCMS-ESI (POS.) m/z: 438.1 [M + H]⁺. ¹H |
| | | | NMR (400 MHz, DMSO-d₆) δ 9.73 (s, 1H), |
| | | | 8.93 (d, J = 2.3 Hz, 1H), 8.85 (d, J = 8.0 Hz, |
| | | | 1H), 8.13 (dd, J = 8.1, 2.4 Hz, 1H), 7.49-7.43 |
| | | | (m, 4H), 7.38 (dd, J = 12.0, 8.3 Hz, 3H), 7.29 |
| | | | (d, J = 8.7 Hz, 2H), 5.13 (s, 2H), 5.10 (p, J = |
| | | | 7.2 Hz, 1H), 2.80 (q, J = 7.6 Hz, 2H), 1.45 |
| | | | (d, J = 7.0 Hz, 3H), 1.23 (t, J = 7.6 Hz, 3H). |
| 110 | 16.2 | 5-methoxy-6- methylnicotinic acid | 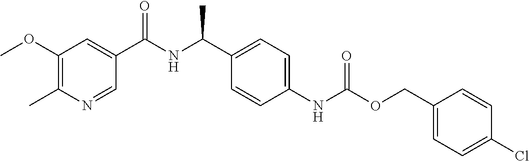 [SMILES: COc1cc(C(=O)N[C@@H](C)c2ccc(NC(=O)OCc3ccc(Cl)cc3)cc2)cnc1C] |
| | | | Compound 110: (4-chlorophenyl)methyl N- |
| | | | {4-[(1S)-1-[(5-methoxy-6-methylpyridin-3- |
| | | | yl)formamido]ethyl]phenyl}carbamate. |
| | | | LCMS-ESI (POS.) m/z: 454.1 [M + H]⁺. 1H |
| | | | NMR (400 MHz, DMSO-d₆) δ 9.74 (s, 1H), |
| | | | 8.84 (d, J = 8.0 Hz, 1H), 8.51 (d, J = 1.8 Hz, |
| | | | 1H), 7.68 (d, J = 1.8 Hz, 1H), 7.48-7.43 (m, |
| | | | 4H), 7.40 (d, J = 8.4 Hz, 2H), 7.30 (d, J = 8.6 Hz, |
| | | | 2H), 5.17-5.09 (m, 3H), 3.87 (s, 3H), |
| | | | 2.40 (s, 3H), 1.47 (d, J = 7.0 Hz, 3H). |
| 181 | 16.2 | 1-isopropyl-1H- pyrazole-5-carboxylic acid | 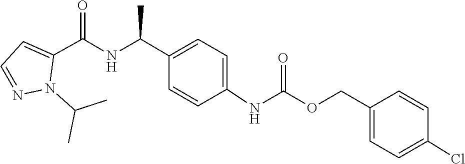 [SMILES: CC(C)n1nccc1C(=O)N[C@@H](C)c1ccc(NC(=O)OCc2ccc(Cl)cc2)cc1] |
| | | | Compound 181: (4-chlorophenyl)methyl N- |
| | | | {4-[(1S)-1-[(2-isopropylpyrazol-3- |
| | | | yl)formamido]ethyl]phenyl}carbamate. |
| | | | LCMS-ESI (POS.) m/z: 441.2 [M + H]+. ¹H |
| | | | NMR (400 MHz, DMSO-d₆) δ 9.73 (s, 1H), |
| | | | 8.54 (d, J = 8.1 Hz, 1H), 7.98 (s, 1H), 7.63 |
| | | | (d, J = 1.0 Hz, 1H), 7.48-7.43 (m, 4H), 7.39 |
| | | | (d, J = 8.3 Hz, 2H), 7.27 (d, J = 8.6 Hz, 2H), |
| | | | 5.17 (sept, J = 6.8 Hz, 1H), 5.13 (s, 2H), 5.04 |
| | | | (p, J = 7.1 Hz, 1H), 1.41 (d, J = 7.0 Hz, 3H), |
| | | | 1.38 (d, J = 6.7 Hz, 3H), 1.36 (d, J = 6.7 Hz, |
| | | | 3H). |
| 290 | 16.2 | 4-cyclopropyloxazole- 5-carboxylic acid | 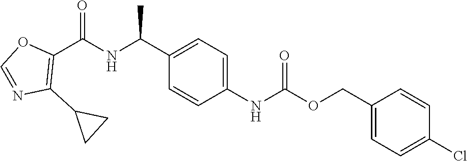 [SMILES: C[C@H](NC(=O)c1ocnc1C1CC1)c1ccc(NC(=O)OCc2ccc(Cl)cc2)cc1] |
| | | | Compound 290: (4-chlorophenyl)methyl N- |
| | | | {4-[(1S)-1-[(4-cyclopropyl-1,3-oxazol-5- |
| | | | yl)formamido]ethyl]phenyl}carbamate. |
| | | | LCMS-APCI (POS.) m/z: 440.3 [M + H]⁺. ¹H |
| | | | NMR (400 MHz, DMSO-d₆) δ 9.73 (s, 1H), |
| | | | 8.69 (d, J = 8.3 Hz, 1H), 8.35 (d, J = 0.6 Hz, |
| | | | 1H), 7.48-7.42 (m, 4H), 7.39 (d, J = 8.4 Hz, |
| | | | 2H), 7.29 (d, J = 8.7 Hz, 2H), 5.13 (s, 2H), |
| | | | 5.08 (p, J = 7.1 Hz, 1H), 2.58 (ddd, J = 10.0, |
| | | | 8.5, 4.9 Hz, 1H), 1.44 (d, J = 7.0 Hz, 3H), 0.98- |
| | | | 0.88 (m, 2H), 0.86-0.76 (m, 2H). |
| 259 | 16.2 | 3-(difluoromethyl)-1- methyl-1H-pyrazole-5- carboxylic acid | 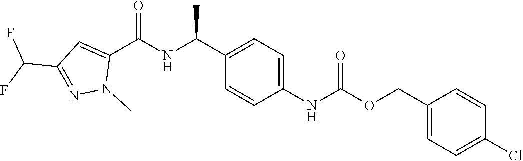 [SMILES: C[C@H](NC(=O)c1cc(C(F)F)nn1C)c1ccc(NC(=O)OCc2ccc(Cl)cc2)cc1] |
| | | | Compound 259: (4-chlorophenyl)methyl N- |
| | | | [4-({[5-(difluoromethyl)-2-methylpyrazol- |
| | | | 3-yl]formamido}methyl)phenyl]carbamate. |
| | | | LCMS-APCI (POS.) m/z: 463.3 [M + H]⁺. ¹H |
| | | | NMR (400 MHz, DMSO-d₆) δ 9.74 (s, 1H), |
| | | | 8.87 (d, J = 8.1 Hz, 1H), 7.48-7.42(m, 4H), |
| | | | 7.40 (d, J = 8.3 Hz, 2H), 7.28 (d, J = 8.7, 2H), |
| | | | 7.24 (s, 1H), 7.02 (t, J = 54.7, 1H), 5.13 (s, |
| | | | 2H), 5.06 (p, J = 7.0 Hz, 1H), 4.05 (s, 3H), |
| | | | 1.43 (d, J = 7.0 Hz, 3H). |
| 100 | 16.2 | 6-methoxynicotinic acid | 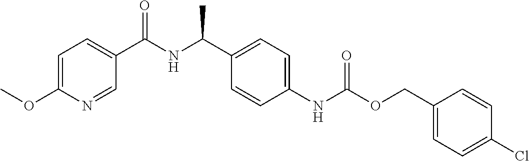 [SMILES: COc1ccc(C(=O)N[C@@H](C)c2ccc(NC(=O)OCc3ccc(Cl)cc3)cc2)cn1] |
| | | | Compound 100: (4-chlorophenyl)methyl N- |
| | | | {4-[(1S)-1-[(6-methoxypyridin-3- |
| | | | yl)formamido]ethyl]phenyl}carbamate. |
| | | | LCMS-APCI (POS.) m/z: 440.2 [M + H]⁺. ¹H |
| | | | NMR (400 MHz, DMSO-d₆) δ 9.74 (s, 1H), |
| | | | 8.73 (d, J = 8.0 Hz, 1H), 8.68 (s, 1H), 8.14 |
| | | | (d, J = 8.9 Hz, 1H), 7.49-7.42 (m, 4H), 7.39 |
| | | | (d, J = 8.3 Hz, 2H), 7.29 (d, J = 8.2 Hz, 2H), |
| | | | 6.88 (d, J = 8.6 Hz, 1H), 5.15-5.05 (m, 3H), |
| | | | 3.90 (s, 3H), 1.44 (d, J = 7.0 Hz, 3H). |
| 213 | 16.2 | 2-methoxy-6- methylnicotinic acid | 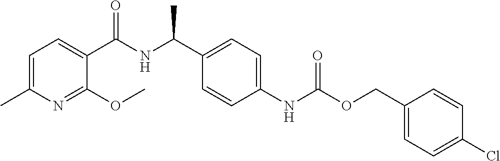 [SMILES: COc1nc(C)ccc1C(=O)N[C@@H](C)c1ccc(NC(=O)OCc2ccc(Cl)cc2)cc1] |
| | | | Compound 213: (4-chlorophenyl)methyl N- |
| | | | {4-[(1S)-1-[(2-methoxy-6-methylpyridin-3- |
| | | | yl)formamido]ethyl]phenyl}carbamate. |
| | | | LCMS-APCI (POS.) m/z: 454.2 [M + H]⁺. ¹H |
| | | | NMR (400 MHz, DMSO-d₆) δ 9.75 (s, 1H), |
| | | | 8.37 (d, J = 7.9 Hz, 1H), 7.97 (d, J = 7.6 Hz, |
| | | | 1H), 7.48-7.44 (m, 4H), 7.41 (d, J = 8.4 Hz, |
| | | | 2H), 7.30 (d, J = 8.2 Hz, 2H), 6.96 (d, J = 7.6 Hz, |
| | | | 1H), 5.13 (s, 2H), 5.06 (p, J = 7.1 Hz, 1H), |
| | | | 3.96 (s, 3H), 2.43 (s, 3H), 1.43 (d, J = 6.9 Hz, |
| | | | 3H). |
| 218 | 16.2 | 2-methyl-1,3-oxazole- 5-carboxylic acid | 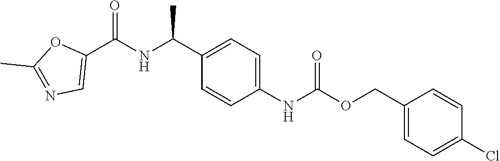 [SMILES: Cc1ncc(C(=O)N[C@@H](C)c2ccc(NC(=O)OCc3ccc(Cl)cc3)cc2)o1] |
| | | | Compound 218: (4-chlorophenyl)methyl N- |
| | | | {4-[(1S)-1-[(2-methyl-1,3-oxazol-5- |
| | | | yl)formamido]ethyl]phenyl}carbamate. |
| | | | LCMS-APCI (POS.) m/z: 414.1 [M + H]⁺. ¹H |
| | | | NMR (400 MHz, DMSO-d₆) δ 9.74 (s, 1H), |
| | | | 8.78 (d, J = 8.0 Hz, 1H), 7.67-7.62 (m, 1H), |
| | | | 7.51-7.34 (m, 6H), 7.28 (d, J = 7.7 Hz, 2H), |
| | | | 5.13 (s, 2H), 5.06 (t, J = 7.2 Hz, 1H), 2.47 (s, |
| | | | 3H), 1.43 (d, J = 7.0 Hz, 3H). |
| 149 | 16.2 | 2-methylpyrazole-3- carboxylic acid | 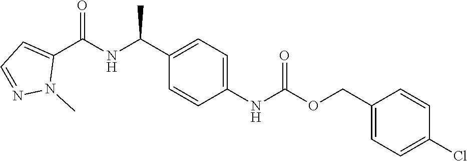 [SMILES: C[C@H](NC(=O)c1ccnn1C)c1ccc(NC(=O)OCc2ccc(Cl)cc2)cc1] |
| | | | Compound 149: (4-chlorophenyl)methyl N- |
| | | | {4-[(1S)-1-[(2-methylpyrazol-3- |
| | | | yl)formamido]ethyl]phenyl}carbamate. |
| | | | LCMS-APCI (POS.) m/z: 413.4 [M + H]⁺. ¹H |
| | | | NMR (400 MHz, DMSO-d6) 8 9.75 (s, 1H), |
| | | | 8.71 (d, J = 8.1 Hz, 1H), 7.48-7.37 (m, 7H), |
| | | | 7.28 (d, J = 7.7 Hz, 2H), 6.95 (s, 1H), 5.13 (s, |
| | | | 2H), 5.10-5.03 (m, 1H), 4.01 (s, 3H), 1.44 (d, |
| | | | 3H). |
| 159 | 16.2 | 5-fluoropyridine-3- carboxylic acid | 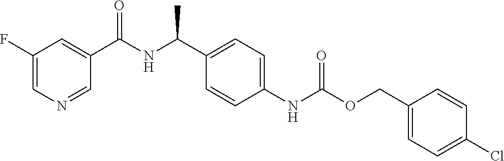 [SMILES: C[C@H](NC(=O)c1cncc(F)c1)c1ccc(NC(=O)OCc2ccc(Cl)cc2)cc1] |
| | | | Compound 159: (4-chlorophenyl)methyl N- |
| | | | {4-[(1S)-1-[(5-fluoropyridin-3- |
| | | | yl)formamido]ethyl]phenyl}carbamate. |
| | | | LCMS-APCI (POS.) m/z: 428.3 [M + H]⁺. ¹H |
| | | | NMR (400 MHz, DMSO-d₆) δ 9.75 (s, 1H), |
| | | | 9.02 (d, J = 7.9 Hz, 1H), 8.91 (s, 1H), 8.74 (s, |
| | | | 1H), 8.12 (d, J = 9.7 Hz, 1H), 7.48-7.38 (m, |
| | | | 6H), 7.31 (d, J = 8.3 Hz, 2H), 5.14 (s, 2H), 5.13- |
| | | | 5.08 (m, 1H), 1.47 (d, J = 7.0 Hz, 3H). |
| 150 | 16.2 | 3-methyl-1,2-oxazole- 4-carboxylic acid | 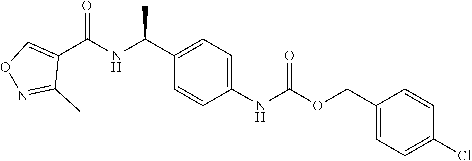 [SMILES: Cc1nocc1C(=O)N[C@@H](C)c1ccc(NC(=O)OCc2ccc(Cl)cc2)cc1] |
| | | | Compound 150: (4-chlorophenyl)methyl N- |
| | | | {4-[(1S)-1-[(3-methyl-1,2-oxazol-4- |
| | | | 16.2yl)formamido]ethyl]phenyl}carbamate. |
| | | | LCMS-APCI (POS.) m/z: 414.4 [M + H]⁺. ¹H |
| | | | NMR (400 MHz, DMSO-d₆) δ 9.75 (s, 1H), |
| | | | 9.32 (s, 1H), 8.62 (d, J = 7.9 Hz, 1H), 7.51- |
| | | | 7.38 (m, 6H), 7.31-7.24 (m, 2H), 5.14 (s, 2H), |
| | | | 5.03 (p, J = 7.1 Hz, 1H), 2.35 (s, 3H), 1.42 (d, |
| | | | J = 7.0 Hz, 3H). |
| 241 | 16.2 | 5-methyl-1,3-oxazole- 4-carboxylic acid | 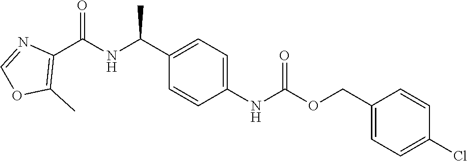 [SMILES: Cc1ocnc1C(=O)N[C@@H](C)c1ccc(NC(=O)OCc2ccc(Cl)cc2)cc1] |
| | | | Compound 241: (4-chlorophenyl)methyl N- |
| | | | {4-[(1S)-1-[(5-methyl-1,3-oxazol-4- |
| | | | yl)formamido]ethyl]phenyl}carbamate. |
| | | | LCMS-ES (POS.) m/z: 415.0 [M + H]⁺. ¹H |
| | | | NMR (400 MHz, DMSO-d₆) δ 9.73 (s, 1H), |
| | | | 8.37-8.29 (m, 2H), 7.51-7.35 (m, 6H), 7.34- |
| | | | 7.26 (m, 2H), 5.13 (s, 2H), 5.05 (p, J = 7.3 Hz, |
| | | | 1H), 2.54 (s, 3H), 1.45 (d, J = 7.1 Hz, 3H). |
| 260 | 16.2 | 4-ethyl-1,3-oxazole-5- carboxylic acid | 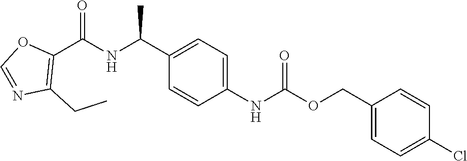 [SMILES: CCc1ncoc1C(=O)N[C@@H](C)c1ccc(NC(=O)OCc2ccc(Cl)cc2)cc1] |
| | | | Compound 260: (4-chlorophenyl)methyl N- |
| | | | {4-[(1S)-1-[(4-ethyl-1,3-oxazol-5- |
| | | | yl)formamido]ethyl]phenyl}carbamate. |
| | | | LCMS-ES (POS.) m/z: 428.1 [M + H]⁺. ¹H |
| | | | NMR (400 MHz, DMSO-d₆) δ 9.74 (s, 1H), |
| | | | 8.74 (d, J = 8.3 Hz, 1H), 8.42 (s, 1H), 7.51- |
| | | | 7.36 (m, 6H), 7.33-7.25 (m, 2H), 5.13 (s, 2H), |
| | | | 5.06 (p, J = 7.2 Hz, 1H), 2.79 (q, J = 7.5 Hz, |
| | | | 2H), 1.44 (d, J = 7.0 Hz, 3H), 1.12 (t, J = 7.5 Hz, |
| | | | 3H). |
| 152 | 16.2 | 4-methyl-1,3-oxazole- 5-carboxylic acid | 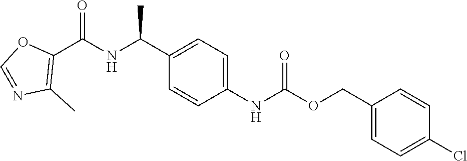 [SMILES: Cc1ncoc1C(=O)N[C@@H](C)c1ccc(NC(=O)OCc2ccc(Cl)cc2)cc1] |
| | | | Compound 152: (4-chlorophenyl)methyl N- |
| | | | {4-[(1S)-1-[(4-methyl-1,3-oxazol-5- |
| | | | yl)formamido]ethyl]phenyl}carbamate. |
| | | | LCMS-APCI (POS.) m/z: 414.3 [M + H]⁺. ¹H |
| | | | NMR (400 MHz, DMSO-d₆) δ 9.74 (s, 1H), |
| | | | 8.74 (d, J = 8.3 Hz, 1H), 8.42 (s, 1H), 7.48- |
| | | | 7.43 (m, 4H), 7.39 (d, J = 8.3 Hz, 2H), 7.33- |
| | | | 7.25 (m, 2H), 5.13 (s, 2H), 5.06 (p, J = 7.3 Hz, |
| | | | 1H), 2.34 (s, 3H), 1.44 (d, J = 7.1 Hz, 3H). |
| 142 | 16.2 | 4-methyl-1,2-oxazole- 5-carboxylic acid | 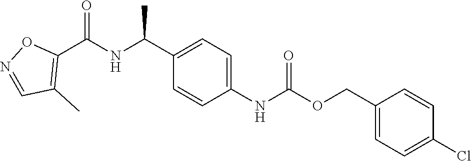 [SMILES: Cc1cnoc1C(=O)N[C@@H](C)c1ccc(NC(=O)OCc2ccc(Cl)cc2)cc1] |
| | | | Compound 142: (4-chlorophenyl)methyl N- |
| | | | {4-[(1S)-1-[(4-methyl-1,2-oxazol-5- |
| | | | yl)formamido]ethyl]phenyl}carbamate. |
| | | | LCMS-APCI (POS.) m/z: 414.4 [M + H]⁺. ¹H |
| | | | NMR (400 MHz, DMSO-d₆) δ 9.74 (s, 1H), |
| | | | 8.74 (d, J = 8.3 Hz, 1H), 8.42 (s, 1H), 7.48- |
| | | | 7.43 (m, 4H), 7.39 (d, J = 8.3 Hz, 2H), 7.33- |
| | | | 7.25 (m, 2H), 5.13 (s, 2H), 5.06 (p, J = 7.3 Hz, |
| | | | 1H), 2.34 (s, 3H), 1.44 (d, J = 7.1 Hz, 3H). |
| 44 | 16.2 | 3-fluoropyridine-4- carboxylic acid | 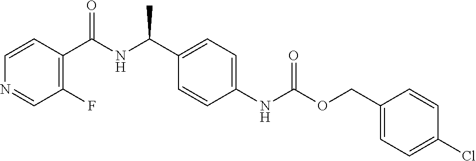 [SMILES: C[C@H](NC(=O)c1ccncc1F)c1ccc(NC(=O)OCc2ccc(Cl)cc2)cc1] |
| | | | Compound 44: (4-chlorophenyl)methyl N- |
| | | | {4-[(1S)-1-[(3-fluoropyridin-4- |
| | | | yl)formamido]ethyl]phenyl}carbamate. |
| | | | LCMS-APCI (POS.) m/z: 428.1 [M + H]⁺. ¹H |
| | | | NMR (400 MHz, DMSO-d₆) δ 9.77 (s, 1H), |
| | | | 9.05 (d, J = 7.9 Hz, 1H), 8.68 (d, J = 1.5 Hz, |
| | | | 1H), 8.52 (dd, J = 4.8, 1.2 Hz, 1H), 7.55 (t, J = |
| | | | 5.9, 4.8 Hz, 1H), 7.51-7.39 (m, 6H), 7.34- |
| | | | 7.26 (m, 2H), 5.14 (s, 2H), 5.07 (p, J = 7.1 Hz, |
| | | | 1H), 1.42 (d, J = 7.0 Hz, 3H). |
| 57 | 16.2 | 3,5-difluoropyridine-4- carboxylic acid | 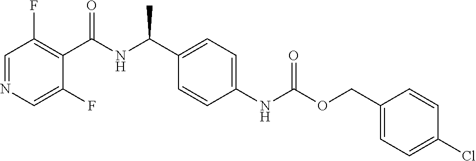 [SMILES: C[C@H](NC(=O)c1c(F)cncc1F)c1ccc(NC(=O)OCc2ccc(Cl)cc2)cc1] |
| | | | Compound 57: (4-chlorophenyl)methyl N- |
| | | | {4-[(1S)-1-[(3,5-difluoropyridin-4- |
| | | | yl)formamido]ethyl]phenyl}carbamate. |
| | | | LCMS-APCI (POS.) m/z: 446.3 [M + H]⁺. ¹H |
| | | | NMR (400 MHz, DMSO-d₆) δ 9.79 (s, 1H), |
| | | | 9.37 (d, J = 7.9 Hz, 1H), 8.64 (s, 2H), 7.49- |
| | | | 7.38 (m, 7H), 7.28 (d, J = 8.4 Hz, 2H), 5.15 (s, |
| | | | 2H), 1.41 (d, J = 6.9 Hz, 3H). |
| 56 | 16.2 | 2-(2- methoxyethyl)pyrazole- 3-carboxylic acid | 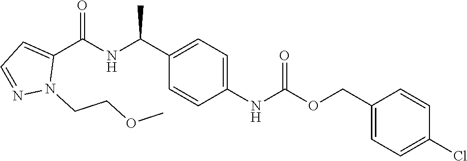 [SMILES: COCCn1nccc1C(=O)N[C@@H](C)c1ccc(NC(=O)OCc2ccc(Cl)cc2)cc1] |
| | | | Compound 56: (4-chlorophenyl)methyl N- |
| | | | {4-[(1S)-1-{[2-(2-methoxyethyl)pyrazol-3- |
| | | | yl]formamido}ethyl]phenyl}carbamate. |
| | | | LCMS-APCI (POS.) m/z: 457.2 [M + H]⁺. ¹H |
| | | | NMR (400 MHz, DMSO-d₆) δ 9.75 (s, 1H), |
| | | | 8.75 (d, J = 8.2 Hz, 1H), 7.51-7.23 (m, 9H), |
| | | | 6.89 (s, 1H), 5.14 (s, 2H), 5.10-5.02 (m, 1H), |
| | | | 4.61 (d, J = 5.7 Hz, 2H), 3.60 (t, J = 5.8 Hz, |
| | | | 2H), 3.14 (s, 3H), 1.44 (d, J = 6.8 Hz, 3H). |
| 210 | 16.2 | 2-cyclobutylpyrazole-3- carboxylic acid | 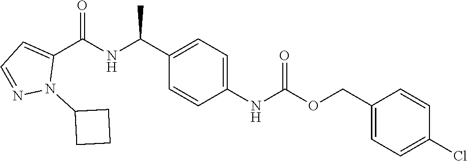 [SMILES: C[C@H](NC(=O)c1ccnn1C1CCC1)c1ccc(NC(=O)OCc2ccc(Cl)cc2)cc1] |
| | | | Compound 210: (4-chlorophenyl)methyl N- |
| | | | {4-[(1S)-1-[(2-cyclobutylpyrazol-3- |
| | | | yl)formamido]ethyl]phenyl}carbamate. |
| | | | LCMS-ES (POS.) m/z: 453.1 [M + H]⁺. ¹H |
| | | | NMR (400 MHz, DMSO-d₆) δ 9.75 (s, 1H), |
| | | | 8.72 (d, J = 8.2 Hz, 1H), 7.53 (s, 1H), 7.48- |
| | | | 7.37 (m, 6H), 7.27 (d, J = 8.2 Hz, 2H), 6.89 (s, |
| | | | 1H), 5.62-5.55 (m, 1H), 5.14 (s, 2H), 5.09- |
| | | | 5.00 (m, 1H), 2.27 (s, 2H), 1.78-1.70 (m, 2H), |
| | | | 1.43 (d, J = 6.9 Hz, 3H), 1.01 (s, 2H). |
| 141 | 16.2 | 4-fluoro-2,5- dimethylpyrazole-3- carboxylic acid | 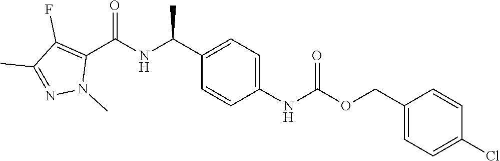 [SMILES: Cc1nn(C)c(C(=O)N[C@@H](C)c2ccc(NC(=O)OCc3ccc(Cl)cc3)cc2)c1F] |
| | | | Compound 141: (4-chlorophenyl)methyl N- |
| | | | {4-[(1S)-1-[(4-fluoro-2,5-dimethylpyrazol- |
| | | | 3-yl)formamido]ethyl]phenyl}carbamate. |
| | | | LCMS-APCI (POS.) m/z: 445.2 [M + H]⁺. ¹H |
| | | | NMR (400 MHz, DMSO-d₆) δ 9.76 (s, 1H), |
| | | | 8.52 (d, J = 8.0 Hz, 1H), 7.49-7.37 (m, 6H), |
| | | | 7.29 (d, J = 8.0 Hz, 2H), 5.14 (s, 2H), 5.06 (p, |
| | | | J = 7.2 Hz, 1H), 3.81 (s, 3H), 2.14 (s, 3H), 1.43 |
| | | | (d, J = 6.9 Hz, 3H). |
| 137 | 16.2 | 5-ethyl-2- methylpyrazole-3- carboxylic acid | 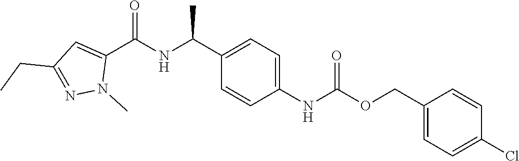 [SMILES: CCc1cc(C(=O)N[C@@H](C)c2ccc(NC(=O)OCc3ccc(Cl)cc3)cc2)n(C)n1] |
| | | | Compound 137: (4-chlorophenyl)methyl N- |
| | | | {4-[(1S)-1-[(5-ethyl-2-methylpyrazol-3- |
| | | | yl)formamido]ethyl]phenyl}carbamate. |
| | | | LCMS-APCI (POS.) m/z: 441.2 [M + H]⁺. ¹H |
| | | | NMR (400 MHz, DMSO-d₆) δ 9.74 (s, 1H), |
| | | | 8.64 (d, J = 8.1 Hz, 1H), 7.48-7.37 (m, 6H), |
| | | | 7.28 (d, J = 8.2 Hz, 2H), 6.77 (s, 1H), 5.14 (s, |
| | | | 2H), 5.05 (t, J = 7.3 Hz, 1H), 3.94 (s, 3H), 2.56 |
| | | | (d, J = 7.8 Hz, 2H), 1.42 (d, J = 7.0 Hz, 3H), |
| | | | 1.17 (t, J = 8.4, 6.8 Hz, 3H). |
| 129 | 16.2 | 2-(2- methylpropyl)pyrazole- | 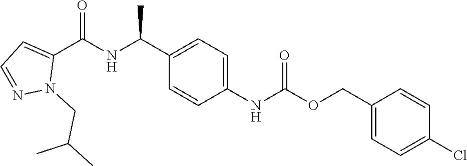 [SMILES: CC(C)Cn1nccc1C(=O)N[C@@H](C)c1ccc(NC(=O)OCc2ccc(Cl)cc2)cc1] |
| | | | Compound 129: (4-chlorophenyl)methyl N- |
| | | | {4-[(1S)-1-{[2-(2-methylpropyl)pyrazol-3- |
| | | | yl]formamido}ethyl]phenyl}carbamate. |
| | | | LCMS-ES (POS.) m/z: 455.1 [M + H]⁺. ¹H |
| | | | NMR (400 MHz, DMSO-d₆) δ 9.74 (s, 1H), |
| | | | 8.73 (d, J = 8.2 Hz, 1H), 7.50-7.22 (m, 9H), |
| | | | 6.91 (s, 1H), 5.14 (s, 2H), 5.10-5.02 (m, 1H), |
| | | | 4.35-4.22 (m, 2H), 2.07-1.99 (m, 1H), 1.43 |
| | | | (d, J = 7.1 Hz, 3H), 0.75 (t, J = 7.4 Hz, 6H). |
| 95 | 16.2 | 3-carboxylic acid 5-methoxy-2- methylpyrazole-3- carboxylic acid | 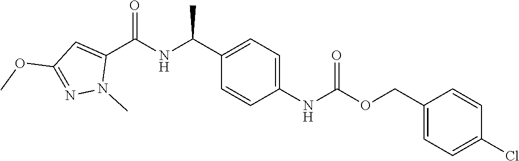 [SMILES: COc1cc(C(=O)N[C@@H](C)c2ccc(NC(=O)OCc3ccc(Cl)cc3)cc2)n(C)n1] |
| | | | Compound 95: (4-chlorophenyl)methyl N- |
| | | | (4-[(1S)-1-[(5-methoxy-2-methylpyrazol-3- |
| | | | yl)formamido]ethyl]phenyl}carbamate. |
| | | | LCMS-APCI (POS.) m/z: 443.1 [M + H]⁺. ¹H |
| | | | NMR (400 MHz, DMSO-d₆) δ 9.75 (s, 1H), |
| | | | 8.65 (d, J = 8.3 Hz, 1H), 7.51-7.34 (m, 6H), |
| | | | 7.27 (d, J = 8.2 Hz, 2H), 6.38 (s, 1H), 5.14 (s, |
| | | | 2H), 5.04 (t, J = 7.5 Hz, 1H), 3.87 (s, 3H), 3.77 |
| | | | (s, 3H), 1.42 (d, J = 7.1 Hz, 3H). |
| 92 | 16.2 | 4-methoxy-2- methylpyrazole-3- carboxylic acid | 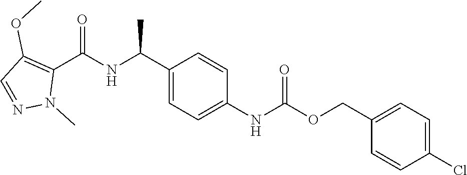 [SMILES: COc1cnn(C)c1C(=O)N[C@@H](C)c1ccc(NC(=O)OCc2ccc(Cl)cc2)cc1] |
| | | | Compound 92: (4-chlorophenyl)methyl N- |
| | | | {4-[(1S)-1-[(4-methoxy-2-methylpyrazol-3- |
| | | | yl)formamido]ethyl]phenyl}carbamate. |
| | | | LCMS-APCI (POS.) m/z: 443.1 [M + H]⁺. ¹H |
| | | | NMR (400 MHz, DMSO-d₆) δ 9.77 (s, 1H), |
| | | | 7.56 (d, J = 7.9 Hz, 1H), 7.49-7.39 (m, 7H), |
| | | | 7.29 (d, J = 8.0 Hz, 2H), 5.14 (s, 2H), 5.06 |
| | | | (t, J = 7.2 Hz, 1H), 3.96 (s, 3H), 3.87 (s, 3H), |
| | | | 1.44 (d, J = 7.0 Hz, 3H). |
| 12 | 16.2 | trimethylpyrazole-3- carboxylic acid | 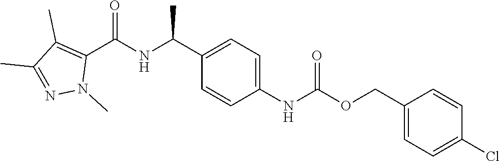 [SMILES: Cc1nn(C)c(C(=O)N[C@@H](C)c2ccc(NC(=O)OCc3ccc(Cl)cc3)cc2)c1C] |
| | | | Compound 12: (4-chlorophenyl)methyl N- |
| | | | {4-[(1S)-1-[(2,4,5-trimethylpyrazol-3- |
| | | | yl)formamido]ethyl]phenyl}carbamate. |
| | | | LCMS-APCI (POS.) m/z: 441.1 [M + H]⁺. ¹H |
| | | | NMR (400 MHz, DMSO-d₆) δ 9.76 (s, 1H), |
| | | | 8.57 (d, J = 8.3 Hz, 1H), 7.49-7.38 (m, 6H), |
| | | | 7.33-7.26 (m, 2H), 5.14 (s, 2H), 5.06 (p, J = |
| | | | 7.4, 6.8 Hz, 1H), 3.71 (s, 3H), 2.07 (s, 3H), |
| | | | 1.98 (s, 3H), 1.42 (d, J = 6.9 Hz, 3H). |
| 20 | 16.2 | 2-(oxan-4-yl)pyrazole- 3-carboxylic acid | 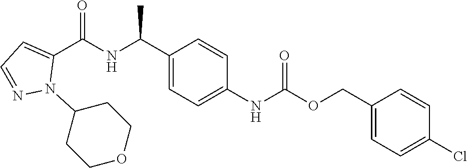 [SMILES: C[C@H](NC(=O)c1ccnn1C1CCOCC1)c1ccc(NC(=O)OCc2ccc(Cl)cc2)cc1] |
| | | | Compound 20: (4-chlorophenyl)methyl N- |
| | | | {4-[(1S)-1-{[2-(oxan-4-yl)pyrazol-3- |
| | | | yl]formamido}ethyl]phenyl}carbamate. |
| | | | LCMS-ES (POS.) m/z: 483.1 [M + H]⁺. ¹H |
| | | | NMR (400 MHz, DMSO-d₆) δ 9.75 (s, 1H), |
| | | | 8.79-8.72 (m, 1H), 7.55-7.49 (m, 1H), 7.49- |
| | | | 7.37 (m, 6H), 7.32-7.24 (m, 2H), 6.93 (s, |
| | | | 1H), 5.29-5.21 (m, 1H), 5.14 (s, 2H), 5.10- |
| | | | 5.03 (m, 1H), 3.93 (s, 2H), 3.44-3.35 (m, 2H), |
| | | | 2.02 (q, J = 12.6 Hz, 2H), 1.80 (t, J = 12.2 Hz, |
| | | | 2H), 1.44 (d, J = 6.8 Hz, 3H). |
| | | | ¹H NMR (400 MHz, Methylene Chloride-d₂) |
| | | | δ 7.52 (s, 1H), 7.45-7.31 (m, 8H), 6.82 (s, |
| | | | 1H), 6.56 (s, 1H), 6.28 (d, J = 7.4 Hz, 1H), |
| | | | 5.34-5.28 (m, 1H), 5.22-5.17 (m, 3H), |
| | | | 4.11-4.01 (m, 2H), 3.54 (q, J = 11.2 Hz, |
| | | | 2H), 2.31-2.18 (m, 2H), 1.93 (d, J = 13.1 Hz, |
| | | | 2H), 1.58 (d, J = 7.1 Hz, 3H). |
| 164 | 16.2 | 6- (difluoromethyl)nicotinic acid | 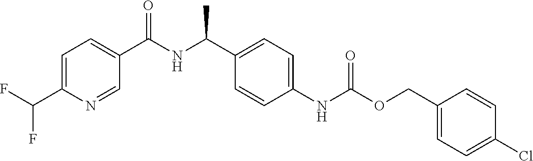 [SMILES: C[C@H](NC(=O)c1ccc(C(F)F)nc1)c1ccc(NC(=O)OCc2ccc(Cl)cc2)cc1] |
| | | | Compound 164: (4-chlorophenyl)methyl N- |
| | | | {4-[(1S)-1-{[6-(difluoromethyl)pyridin-3- |
| | | | yl]formamido}ethyl]phenyl}carbamate. |
| | | | LCMS-APCI (POS.) m/z: 460.2 [M + H]⁺. ¹H |
| | | | NMR (400 MHz, DMSO-d₆) δ 9.74 (s, 1H), |
| | | | 9.11-9.05 (m, 2H), 8.39 (d, J = 8.2 Hz, 1H), |
| | | | 7.81 (d, J = 8.2 Hz, 1H), 7.48-7.43 (m, 4H), |
| | | | 7.41 (d, J = 8.4 Hz, 2H), 7.31 (d, J = 8.4 Hz, |
| | | | 2H), 7.03 (t, J = 54.6 Hz, 1H), 5.17-5.08 (m, |
| | | | 1H), 5.13 (s, 2H), 1.47 (d, J = 7.0 Hz, 3H). |
| 163 | 16.2 | 6-isopropylnicotinic acid | 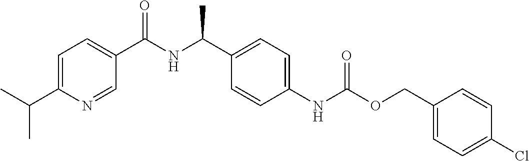 [SMILES: CC(C)c1ccc(C(=O)N[C@@H](C)c2ccc(NC(=O)OCc3ccc(Cl)cc3)cc2)cn1] |
| | | | Compound 163: (4-chlorophenyl)methyl N- |
| | | | {4-[(1S)-1-[(6-isopropylpyridin-3- |
| | | | yl)formamido]ethyl]phenyl}carbamate. |
| | | | LCMS-APCI (POS.) m/z: 452.2 [M + H]⁺. ¹H |
| | | | NMR (400 MHz, DMSO-d₆) δ 9.73 (s, 1H), |
| | | | 8.93 (s, 1H), 8.85 (d, J = 8.1 Hz, 1H), 8.15 |
| | | | (d, J = 8.2 Hz, 1H), 7.48-7.43 (m, 4H), 7.42- |
| | | | 7.37 (m, 3H), 7.29 (d, J = 8.4 Hz, 2H), 5.16- |
| | | | 5.07 (m, 3H), 3.08 (p, J = 6.9 Hz, 1H), 1.45 |
| | | | (d, J = 7.0 Hz, 3H), 1.25 (s, 3H), 1.23 (s, 3H). |
| 60 | 16.2 | 6-methylpyridine-3- carboxylic acid | 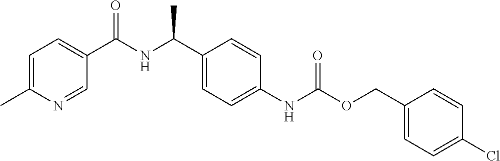 [SMILES: Cc1ccc(C(=O)N[C@@H](C)c2ccc(NC(=O)OCc3ccc(Cl)cc3)cc2)cn1] |
| | | | Compound 60: 4-chlorobenzyl (S)-(4-(1-(6- |
| | | | methylnicotinamido)ethyl)phenyl)carbamate. |
| | | | LCMS- APCI (POS.) m/z: 424.1 [M + H]⁺. ¹H |
| | | | NMR (400 MHz, DMSO-d₆) δ 9.74 (s, 1H), |
| | | | 8.93 (d, J = 2.1 Hz, 1H), 8.89 (d, J = 8.0 Hz, |
| | | | 1H), 8.18 (d, J = 8.2 Hz, 1H), 7.45 (s, 3H), 7.44- |
| | | | 7.37 (m, 3H), 7.30 (d, J = 8.3 Hz, 2H), 5.13 |
| | | | (s, 2H), 2.54 (s, 3H), 1.45 (d, J = 7.0 Hz, 3H), |
| | | | 1.26 (t, J = 6.0 Hz, 2H). |
| 117 | 16.2 | tetrahydro-2H-pyran-4- carboxylic acid | 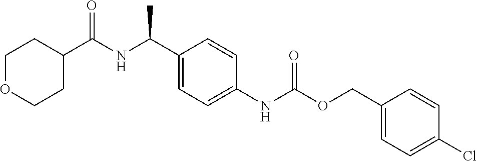 [SMILES: C[C@H](NC(=O)C1CCOCC1)c1ccc(NC(=O)OCc2ccc(Cl)cc2)cc1] |
| | | | Compound 117: 4-chlorobenzyl (S)-(4-(1- |
| | | | (tetrahydro-2H-pyran-4- |
| | | | carboxamido)ethyl)phenyl)carbamate. |
| | | | LCMS- APCI (POS.) m/z: 417.1 [M + H]⁺. ¹H |
| | | | NMR (400 MHz, DMSO-d₆) δ 9.72 (s, 1H), |
| | | | 8.13 (d, J = 8.0 Hz, 1H), 7.45 (s, 4H), 7.37 (d, |
| | | | J = 8.2 Hz, 2H), 7.21-7.16 (m, 2H), 5.13 (d, |
| | | | J = 1.9 Hz, 2H), 4.83 (p, J = 7.2 Hz, 1H), 3.85 |
| | | | (dd, J = 10.4, 5.0 Hz, 2H), 3.26 (dd, J = 10.9, |
| | | | 3.8 Hz, 2H), 1.55 (pd, J = 10.7, 4.3 Hz, 5H), |
| | | | 1.29 (d, J = 7.0 Hz, 3H). |
| 122 | 16.2 | oxetane-3-carboxylic acid | 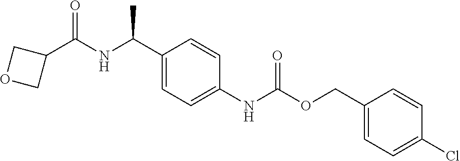 [SMILES: C[C@H](NC(=O)C1COC1)c1ccc(NC(=O)OCc2ccc(Cl)cc2)cc1] |
| | | | Compound 122: 4-chlorobenzyl (S)-(4-(1- |
| | | | (oxetane-3- |
| | | | carboxamido)ethyl)phenyl)carbamate. |
| | | | LCMS- APCI (POS.) m/z: 389.0 [M + H]⁺. ¹H |
| | | | NMR (400 MHz, DMSO-d₆) δ 9.73 (s, 1H), |
| | | | 8.23 (d, J = 8.0 Hz, 1H), 7.45 (s, 4H), 7.38 (d, |
| | | | J = 8.2 Hz, 2H), 7.19 (d, J = 8.3 Hz, 2H), 5.13 |
| | | | (s, 2H), 4.88 (t, J = 7.2 Hz, 1H), 4.63-4.57 |
| | | | (m, 2H), 4.54 (t, J = 6.2 Hz, 1H), 3.79-3.70 |
| | | | (m, 1H), 3.38 (q, J = 7.0 Hz, 1H), 1.30 (d, J = |
| | | | 7.0 Hz, 3H), 1.09 (t, J = 7.1 Hz, 1H). |
| 189 | 16.2 | 3-methyloxetane-3- carboxylic acid | 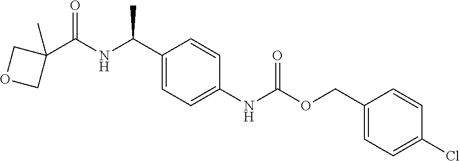 [SMILES: C[C@H](NC(=O)C1(C)COC1)c1ccc(NC(=O)OCc2ccc(Cl)cc2)cc1] |
| | | | Compound 189: 4-chlorobenzyl (S)-(4-(1- |
| | | | (3-methyloxetane-3- |
| | | | carboxamido)ethyl)phenyl)carbamate. |
| | | | LCMS- APCI (POS.) m/z: 403.1 [M + H]⁺. 1H |
| | | | NMR (400 MHz, DMSO-d6) δ 9.73 (s, 1H), |
| | | | 8.13 (d, J = 7.9 Hz, 1H), 7.45 (s, 4H), 7.39 (d, |
| | | | J = 8.2 Hz, 2H), 7.20 (d, J = 8.1 Hz, 2H), 5.13 |
| | | | (s, 2H), 4.88 (t, J = 7.3 Hz, 1H), 4.69 (d, J = |
| | | | 6.0 Hz, 2H), 4.24 (t, J = 4.9 Hz, 2H), 1.48 (s, |
| | | | 3H), 1.33 (d, J = 7.0 Hz, 3H). |
| 246 | 16.2 | oxazole-5-carboxylic acid | 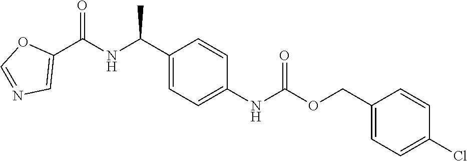 [SMILES: C[C@H](NC(=O)c1cnco1)c1ccc(NC(=O)OCc2ccc(Cl)cc2)cc1] |
| | | | Compound 246: 4-chlorobenzyl (S)-(4-(1- |
| | | | (oxazole-5- |
| | | | carboxamido)ethyl)phenyl)carbamate. |
| | | | LCMS- APCI (POS.) m/z: 400.1 [M + H]⁺. 1H |
| | | | NMR (400 MHz, DMSO-d6) δ 9.75 (s, 1H), |
| | | | 8.94 (d, J = 8.2 Hz, 1H), 8.55 (s, 1H), 7.80 (s, |
| | | | 1H), 7.45 (s, 4H), 7.40 (d, J = 8.3 Hz, 2H), |
| | | | 7.28 (d, J = 8.2 Hz, 2H), 5.13 (s, 2H), 5.06 (t, |
| | | | J = 7.5 Hz, 1H), 1.43 (d, J = 7.0 Hz, 3H). |
| 125 | 16.2 | 2,4-dimethyloxazole-5- carboxylic acid | 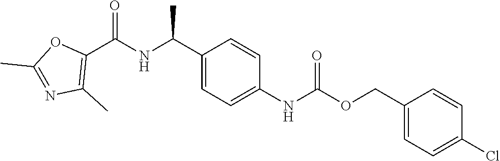 [SMILES: Cc1nc(C)c(C(=O)N[C@@H](C)c2ccc(NC(=O)OCc3ccc(Cl)cc3)cc2)o1] |
| | | | Compound 125: 4-chlorobenzyl (S)-(4-(1- |
| | | | (2,4-dimethyloxazole-5- |
| | | | carboxamido)ethyl)phenyl)carbamate. |
| | | | LCMS- APCI (POS.) m/z: 428.1 [M + H]⁺. 1H |
| | | | NMR (400 MHz, DMSO-d6) δ 9.74 (s, 1H), |
| | | | 8.58 (d, J = 8.3 Hz, 1H), 7.45 (s, 4H), 7.38 (d, |
| | | | J = 8.2 Hz, 2H), 7.28 (d, J = 8.2 Hz, 2H), 5.12 |
| | | | (s, 2H), 5.03 (t, J = 7.5 Hz, 1H), 2.43 (s, 3H), |
| | | | 2.27 (s, 3H), 1.42 (d, J = 7.1 Hz, 3H). |
| 46 | 16.2 | 3,4-dimethylisoxazole- 5-carboxylic acid | 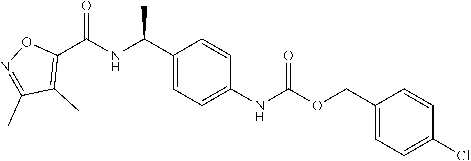 [SMILES: Cc1noc(C(=O)N[C@@H](C)c2ccc(NC(=O)OCc3ccc(Cl)cc3)cc2)c1C] |
| | | | Compound 46: 4-chlorobenzyl (S)-(4-(1- |
| | | | (3,4-dimethylisoxazole-5- |
| | | | carboxamido)ethyl)phenyl)carbamate. |
| | | | LCMS- APCI (POS.) m/z: 428.1 [M + H]⁺. |
| | | | 1H NMR (400 MHz, DMSO-d6) δ 9.77 (s, |
| | | | 1H), 8.38 (d, J = 8.0 Hz, 1H), 7.45 (s, 4H), 7.41 |
| | | | (d, J = 9.0 Hz, 2H), 7.28 (d, J = 8.4 Hz, 2H), |
| | | | 5.13 (s, 2H), 5.02 (t, J = 7.3 Hz, 1H), 2.46 (s, |
| | | | 3H), 2.25 (s, 3H), 1.40 (d, J = 7.0 Hz, 3H). |
| 200 | 16.2 | 3-methylisoxazole-5- carboxylic acid | 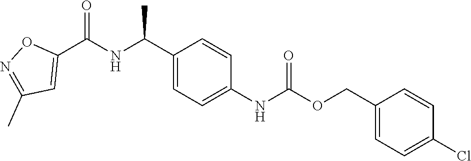 [SMILES: Cc1cc(C(=O)N[C@@H](C)c2ccc(NC(=O)OCc3ccc(Cl)cc3)cc2)on1] |
| | | | Compound 200: 4-chlorobenzyl (S)-(4-(1- |
| | | | (3-methylisoxazole-5- |
| | | | carboxamido)ethyl)phenyl)carbamate. |
| | | | LCMS- APCI (POS.) m/z: 414.0 [M + H]⁺. |
| | | | 1H NMR (400 MHz, DMSO-d6) δ 9.76 (s, |
| | | | 1H), 9.22 (d, J = 8.5 Hz, 1H), 7.45 (s, 4H), 7.40 |
| | | | (d, J = 8.4 Hz, 2H), 7.28 (d, J = 8.2 Hz, 2H), |
| | | | 6.93 (s, 1H), 5.13 (s, 2H), 5.05 (t, J = 7.2 Hz, |
| | | | 1H), 2.28 (s, 3H), 1.44 (d, J = 6.9 Hz, 3H). |
| 267 | 16.2 | 5-methyloxazole-2- carboxylic acid | 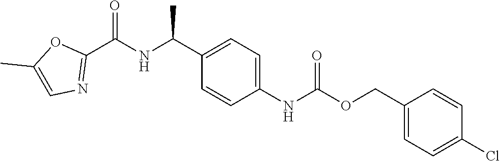 [SMILES: Cc1cnc(C(=O)N[C@@H](C)c2ccc(NC(=O)OCc3ccc(Cl)cc3)cc2)o1] |
| | | | Compound 267: 4-chlorobenzyl (S)-(4-(1- |
| | | | (5-methyloxazole-2- |
| | | | carboxamido)ethyl)phenyl)carbamate. |
| | | | LCMS- APCI (POS.) m/z: 414.0 [M + H]⁺. 1H |
| | | | NMR (400 MHz, DMSO-d6) δ 9.73 (s, 1H), |
| | | | 9.11 (d, J = 8.5 Hz, 1H), 7.45 (d, J = 2.3 Hz, |
| | | | 4H), 7.38 (d, J = 8.2 Hz, 2H), 7.29 (d, J = 8.2 Hz, |
| | | | 2H), 7.06 (s, 1H), 5.13 (s, 2H), 5.04 (t, J = |
| | | | 7.5 Hz, 1H), 2.36 (s, 3H), 1.44 (d, J = 7.0 Hz, |
| | | | 3H). |
| 224 | 16.2 | 2-(oxazol-2-yl)acetic acid | 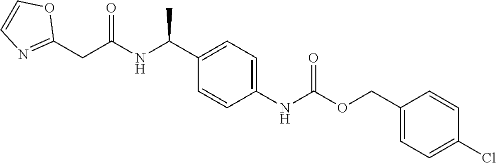 [SMILES: C[C@H](NC(=O)Cc1ncco1)c1ccc(NC(=O)OCc2ccc(Cl)cc2)cc1] |
| | | | Compound 224: 4-chlorobenzyl (S)-(4-(1- |
| | | | (2-(oxazol-2- |
| | | | yl)acetamido)ethyl)phenyl)carbamate. |
| | | | LCMS- APCI (POS.) m/z: 414.1 [M + H]⁺. 1H |
| | | | NMR (400 MHz, DMSO-d6) δ 9.74 (s, 1H), |
| | | | 8.59 (d, J = 7.9 Hz, 1H), 8.01 (s, 1H), 7.45 (s, |
| | | | 4H), 7.39 (d, J = 8.1 Hz, 2H), 7.22 (d, J = 8.2 Hz, |
| | | | 2H), 7.12 (s, 1H), 5.13 (s, 2H), 4.87-4.81 |
| | | | (m, 1H), 3.71 (s, 2H), 1.34 (d, J = 7.1 Hz, 3H). |
| 243 | 16.2 | 4-methyloxazole-2- carboxylic acid | 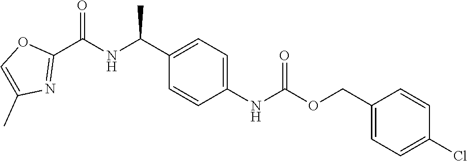 [SMILES: Cc1coc(C(=O)N[C@@H](C)c2ccc(NC(=O)OCc3ccc(Cl)cc3)cc2)n1] |
| | | | Compound 243: 4-chlorobenzyl (S)-(4-(1- |
| | | | (4-methyloxazole-2- |
| | | | carboxamido)ethyl)phenyl)carbamate. |
| | | | LCMS- APCI (POS.) m/z: 414.0 [M + H]⁺. 1H |
| | | | NMR (400 MHz, DMSO-d6) δ 9.73 (s, 1H), |
| | | | 9.20 (d, J = 8.4 Hz, 1H), 7.99 (s, 1H), 7.45 (s, |
| | | | 4H), 7.38 (d, J = 8.4 Hz, 2H), 7.29 (d, J = 8.3 |
| | | | Hz, 2H), 5.12 (s, 2H), 5.06-5.00 (m, 1H), |
| | | | 2.16 (s, 3H), 1.44 (d, J = 7.0 Hz, 3H). |
| 206 | 17.2 | 3,5-dimethyl-1H- pyrazole-4-carboxylic acid | 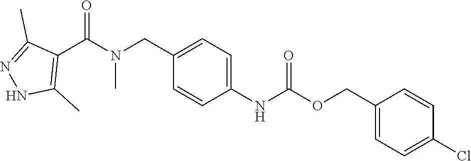 [SMILES: Cc1n[nH]c(C)c1C(=O)N(C)Cc1ccc(NC(=O)OCc2ccc(Cl)cc2)cc1] |
| | | | Compound 206: (4-chlorophenyl)methyl N- |
| | | | (4-{[1-(3,5-dimethyl-1H-pyrazol-4-yl)-N- |
| | | | methylformamido]methyl}phenyl)carbamate. |
| | | | LCMS-APCI (POS.) m/z: 427.3 [M + H]⁺. |
| | | | ¹H NMR (400 MHz, DMSO-d₆) δ 12.44 (bs, |
| | | | 1H), 9.80 (s, 1H), 7.50-7.39 (m, 6H), 7.14 |
| | | | (bs, 2H), 5.13 (s, 2H), 4.49 (bs, 2H), 2.77 (s, |
| | | | 3H), 2.12 (s, 6H). |
| 71 | 17.2 | 4-fluoro-1H-pyrazole-3- carboxylic acid | 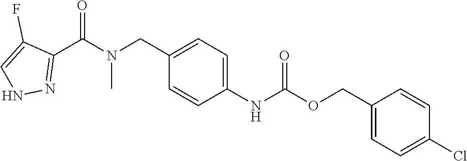 [SMILES: CN(Cc1ccc(NC(=O)OCc2ccc(Cl)cc2)cc1)C(=O)c1n[nH]cc1F] |
| | | | Compound 71: (4-chlorophenyl)methyl N- |
| | | | (4-{[1-(4-fluoro-1H-pyrazol-3-yl)-N- |
| | | | methylformamido]methyl}phenyl)carbamate. |
| | | | LCMS-APCI (POS.) m/z: 417.3 [M + H]⁺. |
| | | | ¹H NMR (400 MHz, 80° C., DMSO-d₆) δ 12.93 |
| | | | (s, 1H), 9.52 (s, 1H), 7.83 (bs, 1H), 7.52-7.33 |
| | | | (m, 6H), 7.25-7.12 (m, 2H), 5.15 (s, 2H), 4.64 |
| | | | (bs, 2H), 2.95 (bs, 3H). |
| 91 | 17.2 | 1H-pyrazole-5- carboxylic acid | 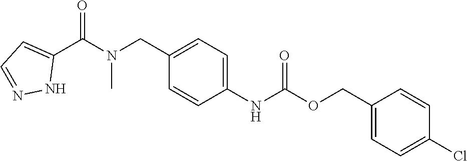 [SMILES: CN(Cc1ccc(NC(=O)OCc2ccc(Cl)cc2)cc1)C(=O)c1ccn[nH]1] |
| | | | Compound 91: (4-chlorophenyl)methyl N- |
| | | | {4-[(N-methyl-1-2H-pyrazol-3- |
| | | | ylformamido)methyl]phenyl}carbamate. |
| | | | LCMS-APCI (POS.) m/z: 399.3 [M + H]⁺. ¹H |
| | | | NMR (400 MHz, 80° C., DMSO-d₆) δ 12.95 |
| | | | (bs, 1H), 9.50 (s, 1H), 7.72 (s, 1H), 7.47- |
| | | | 7.39 (m, 6H), 7.19 (d, J = 8.1 Hz, 2H), 6.58 |
| | | | (s, 1H), 5.15 (s, 2H), 4.69 (bs, 2H), 3.00 (bs, |
| | | | 3H). |
| 85 | 17.2 | 2-methyl-1,3-oxazole- 5-carboxylic acid | 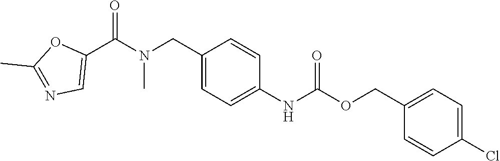 [SMILES: Cc1ncc(C(=O)N(C)Cc2ccc(NC(=O)OCc3ccc(Cl)cc3)cc2)o1] |
| | | | Compound 85: (4-chlorophenyl)methyl N- |
| | | | (4-{[N-methyl-1-(2-methyl-1,3-oxazol-5- |
| | | | yl)formamido]methyl}phenyl)carbamate. |
| | | | LCMS-APCI (POS.) m/z: 414.1 [M + H]⁺. ¹H |
| | | | NMR (400 MHz, DMSO-d₆, temperature = |
| | | | 354 K) δ 9.54 (s, 1H), 7.50 (s, 1H), 7.47-7.42 |
| | | | (m, 6H), 7.19 (d, J = 8.1 Hz, 2H), 5.16 (s, 2H), |
| | | | 4.64 (s, 2H), 3.05 (s, 3H), 2.47 (s, 3H). |
| 50 | 17.2 | 2-methylpyrazole-3- carboxylic acid | 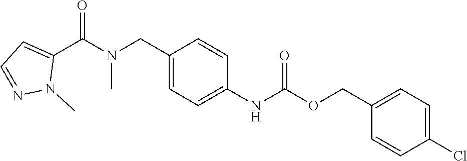 [SMILES: CN(Cc1ccc(NC(=O)OCc2ccc(Cl)cc2)cc1)C(=O)c1ccnn1C] |
| | | | Compound 50: (4-chlorophenyl)methyl N- |
| | | | (4-{[N-methyl-1-(2-methylpyrazol-3- |
| | | | yl)formamido]methyl}phenyl)carbamate. |
| | | | LCMS-APCI (POS.) m/z: 413.3 [M + H]+ . . ¹H |
| | | | NMR (400 MHz, DMSO-d₆) δ 9.82 (s, 1H), |
| | | | 7.49-7.44 (m, 6H), 7.28-7.23 (m, 1H), 7.10- |
| | | | 7.05 (m, 1H), 6.56 (s, 1H), 6.37 (s, 1H), 5.15 |
| | | | (s, 2H), 4.57 (d, J = 26.7 Hz, 2H), 3.84 (d, J = |
| | | | 16.7 Hz, 3H), 2.93 (s, 3H). |
| 250 | 17.2 | 5-fluoropyridine-3- carboxylic acid | 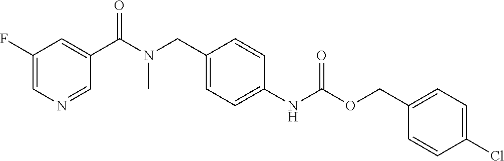 [SMILES: CN(Cc1ccc(NC(=O)OCc2ccc(Cl)cc2)cc1)C(=O)c1cncc(F)c1] |
| | | | Compound 250: (4-chlorophenyl)methyl N- |
| | | | (4-{[1-(5-fluoropyridin-3-yl)-N- |
| | | | methylformamido]methyl}phenyl)carbamate. |
| | | | LCMS-ES (POS.) m/z: 428.0 [M + H]⁺. ¹H |
| | | | NMR (400 MHz, DMSO-d₆, temp = 354 K) δ |
| | | | 9.55 (s, 1H), 8.63 (s, 1H), 8.52 (s, 1H), 7.81 (d, |
| | | | J = 9.3 Hz, 1H), 7.51-7.42 (m, 6H), 7.22 (s, |
| | | | 2H), 5.16 (s, 2H), 4.55 (s, 2H), 2.89 (s, 3H). |
| 108 | 17.2 | 3-methyl-1,2-oxazole- 4-carboxylic acid | 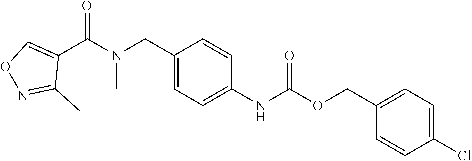 [SMILES: Cc1nocc1C(=O)N(C)Cc1ccc(NC(=O)OCc2ccc(Cl)cc2)cc1] |
| | | | Compound 108: (4-chlorophenyl)methyl N- |
| | | | (4-{[N-methyl-1-(3-methyl-1,2-oxazol-4- |
| | | | yl)formamido]methyl}phenyl)carbamate. |
| | | | LCMS-APCI (POS.) m/z: 414.3 [M + H]⁺. ¹H |
| | | | NMR (400 MHz, DMSO-d₆ temp = 354 K) δ |
| | | | 9.54 (s, 1H), 9.04 (s, 1H), 7.51-7.42 (m, 6H), |
| | | | 7.18 (d, J = 8.1 Hz, 2H), 5.16 (s, 2H), 4.58 (s, |
| | | | 2H), 2.96 (s, 3H), 2.30 (s, 3H). |
| 340 | 17.2 | 5-methyl-1,3-oxazole- 4-carboxylic acid | 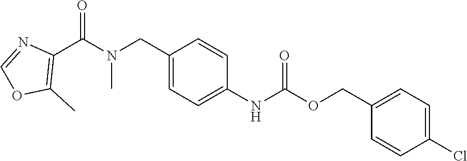 [SMILES: Cc1ocnc1C(=O)N(C)Cc1ccc(NC(=O)OCc2ccc(Cl)cc2)cc1] |
| | | | Compound 340: (4-chlorophenyl)methyl N- |
| | | | (4-{[N-methyl-1-(5-methyl-1,3-oxazol-4- |
| | | | yl)formamido]methyl}phenyl)carbamate. |
| | | | LCMS-APCI (POS.) m/z: 414.8 [M + H]⁺. ¹H |
| | | | NMR (400 MHz, DMSO-d₆ temp = 354 K) δ |
| | | | 9.51 (s, 1H), 8.21 (s, 1H), 7.46-7.40 (m, 6H), |
| | | | 7.19 (d, J = 8.2 Hz, 2H), 5.16 (s, 2H), 4.72 (bs, |
| | | | 2H), 3.00 (bs, 3H), 2.48 (s, 3H). |
| 176 | 17.2 | 4-ethyl-1,3-oxazole-5- carboxylic acid | 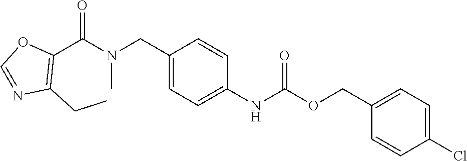 [SMILES: CCc1ncoc1C(=O)N(C)Cc1ccc(NC(=O)OCc2ccc(Cl)cc2)cc1] |
| | | | Compound 176: (4-chlorophenyl)methyl N- |
| | | | (4-{[1-(4-ethyl-1,3-oxazol-5-yl)-N- |
| | | | methylformamido]methyl}phenyl)carbamate. |
| | | | LCMS-APCI (POS.) m/z: 428.3 [M + H]⁺. |
| | | | ¹H NMR (400 MHz, DMSO-d₆, temp = 354 K) |
| | | | δ 9.53 (s, 1H), 8.28 (s, 1H), 7.49-7.42 (m, |
| | | | 6H), 7.19 (d, J = 8.2 Hz, 2H), 5.16 (s, 2H), 4.59 |
| | | | (s, 2H), 2.97 (s, 3H), 2.71 (q, J = 7.5 Hz, 2H), |
| | | | 1.18 (t, J = 7.5 Hz, 3H). |
| 36 | 17.2 | 3-fluoropyridine-4- carboxylic acid | 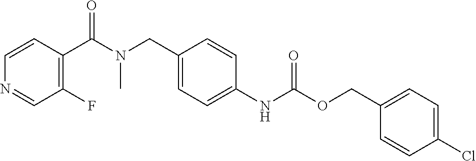 [SMILES: CN(Cc1ccc(NC(=O)OCc2ccc(Cl)cc2)cc1)C(=O)c1ccncc1F] |
| | | | Compound 36: (4-chlorophenyl)methyl N- |
| | | | (4-{[1-(3-fluoropyridin-4-yl)-N- |
| | | | methylformamido]methyl}phenyl)carbamate. |
| | | | LCMS-APCI (POS.) m/z: 428.3 [M + H]⁺. |
| | | | ¹H NMR (400 MHz, DMSO-d₆, temp = 354 K) |
| | | | δ 9.56 (s, 1H), 8.69-8.64 (m, 1H), 8.52 (d, J = |
| | | | 13.6 Hz, 1H), 7.52-7.43 (m, 7H), 7.27 (d, J = |
| | | | 7.9 Hz, 1H), 7.07 (d, J = 8.1 Hz, 1H), 5.16 |
| | | | (s, 2H), 4.66 (s, 1H), 4.33 (s, 1H), 2.95 (s, 1H), |
| | | | 2.77 (s, 2H). |
| | | | NB: rotamers observed. 4.66 and 4.33 are a |
| | | | methylene group with 2H, 2.95 and 2.77 are |
| | | | the N-methyl group with 3H. |
| 55 | 17.2 | 3,5-difluoropyridine-4- carboxylic acid | 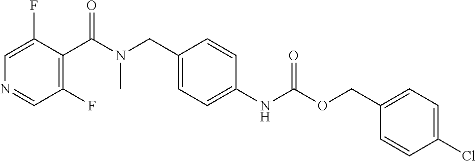 [SMILES: CN(Cc1ccc(NC(=O)OCc2ccc(Cl)cc2)cc1)C(=O)c1c(F)cncc1F] |
| | | | Compound 55: (4-chlorophenyl)methyl N- |
| | | | (4-{[1-(3,5-difluoropyridin-4-yl)-N- |
| | | | methylformamido]methyl}phenyl)carbamate. |
| | | | LCMS-ES (POS.) m/z: 446.1 [M + H]⁺. ¹H |
| | | | NMR (400 MHz, DMSO-d₆) δ 9.58 (s, 1H), |
| | | | 8.62 (d, J = 14.5 Hz, 2H), 7.52-7.42 (m, 6H), |
| | | | 7.26 (d, J = 8.1 Hz, 1H), 7.05 (d, J = 8.1 Hz, |
| | | | 1H), 5.19-5.13 (m, 2H), 4.68 (s, 1H), 4.38 (s, |
| | | | 1H), 2.99 (s, 1H), 2.81 (s, 2H). |
| | | | NB: Rotamers observed. Peaks at 2.99 and |
| | | | 2.81 together make up the N-methyl group. |
| 131 | 17.2 | 2-isopropylpyrazole-3- carboxylic acid | 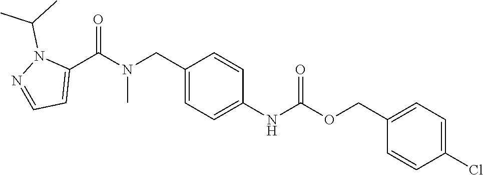 [SMILES: CC(C)n1nccc1C(=O)N(C)Cc1ccc(NC(=O)OCc2ccc(Cl)cc2)cc1] |
| | | | Compound 131: (4-chlorophenyl)methyl N- |
| | | | (4-{[1-(2-isopropylpyrazol-3-yl)-N- |
| | | | methylformamido]methyl}phenyl)carbamate. |
| | | | LCMS-APCI (POS.) m/z: 441.3 [M + H]⁺. |
| | | | ¹H NMR (400 MHz, DMSO-d₆, temp = 353 K) |
| | | | δ 9.56 (s, 1H), 7.51-7.40 (m, 7H), 7.18 (s, |
| | | | 2H), 6.38 (s, 1H), 5.16 (s, 2H), 4.67 (p, J = |
| | | | 6.6 Hz, 1H), 4.58 (s, 2H), 2.92 (s, 3H), 1.40 (d, |
| | | | J = 6.7 Hz, 6H). |
| 77 | 17.2 | 2-(2- methoxyethyl)pyrazole- 3-carboxylic acid | 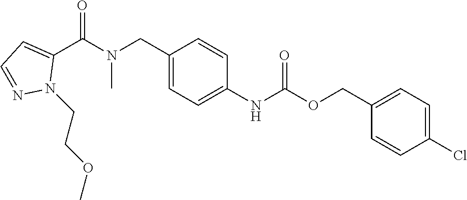 [SMILES: COCCn1nccc1C(=O)N(C)Cc1ccc(NC(=O)OCc2ccc(Cl)cc2)cc1] |
| | | | Compound 77: (4-chlorophenyl)methyl N- |
| | | | [4-({1-[2-(2-methoxyethyl)pyrazol-3-yl]-N- |
| | | | methylformamido}methyl)phenyl]carbamate. |
| | | | LCMS-APCI (POS.) m/z: 457.1 [M + H]⁺. |
| | | | ¹H NMR (400 MHz, DMSO-d₆) δ 9.81 (s, 1H), |
| | | | 7.54-7.43 (m, 6H), 7.26 (d, J = 8.0 Hz, 1H), |
| | | | 7.18-7.12 (m, 1H), 6.54 (s, 2H), 5.15 (s, 2H), |
| | | | 4.56 (d, J = 31.0 Hz, 2H), 4.39 (s, 2H), 3.65- |
| | | | 3.55 (m, 2H), 3.15 (d, J = 37.7 Hz, 3H), 2.91 |
| | | | (s, 3H). Rotamers observed. |
| 74 | 17.2 | 2-cyclobutylpyrazole-3- carboxylic acid | 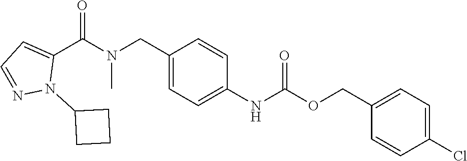 [SMILES: CN(Cc1ccc(NC(=O)OCc2ccc(Cl)cc2)cc1)C(=O)c1ccnn1C1CCC1] |
| | | | Compound 74: (4-chlorophenyl)methyl N- |
| | | | (4-{[1-(2-cyclobutylpyrazol-3-yl)-N- |
| | | | methylformamido]methyl}phenyl)carbamate. |
| | | | LCMS-APCI (POS.) m/z: 453.2 [M + H]⁺. |
| | | | ¹H NMR (400 MHz, DMSO-d₆, temp = 353 K) |
| | | | δ 9.55 (s, 1H), 7.53-7.42 (m, 7H), 7.17 (s, |
| | | | 2H), 6.41 (s, 1H), 5.16 (s, 2H), 4.94-4.85 (m, |
| | | | 1H), 4.55 (s, 2H), 2.90 (s, 3H), 2.61-2.52 (m, |
| | | | 2H), 2.36-2.26 (m, 2H), 1.80 (q, J = 9.3 Hz, |
| | | | 2H). |
| 188 | 17.2 | 4-fluoro-2,5- dimethylpyrazole-3- carboxylic acid | 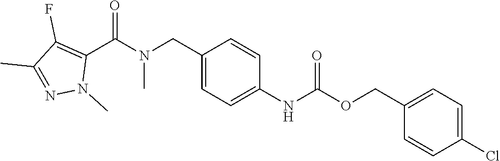 [SMILES: Cc1nn(C)c(C(=O)N(C)Cc2ccc(NC(=O)OCc3ccc(Cl)cc3)cc2)c1F] |
| | | | Compound 188: (4-chlorophenyl)methyl N- |
| | | | (4-{[1-(4-fluoro-2,5-dimethylpyrazol-3-yl)- |
| | | | N- |
| | | | methylformamido]methyl}phenyl)carbamate. |
| | | | LCMS-ES (POS.) m/z: 445.1 [M + H]⁺. ¹H |
| | | | NMR (400 MHz, DMSO-d₆, temp = 353 K) δ |
| | | | 9.55 (s, 1H), 7.50-7.42 (m, 5H), 7.27-7.08 |
| | | | (m, 3H), 5.16 (s, 2H), 4.58 (s, 2H), 3.72 (s, |
| | | | 3H), 2.93 (s, 3H), 2.15 (s, 3H). |
| 289 | 17.2 | 5-ethyl-2- methylpyrazole-3- carboxylic acid | 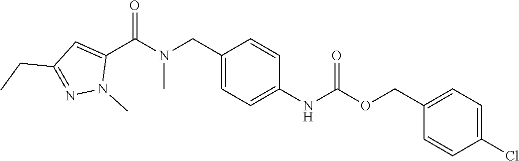 [SMILES: CCc1cc(C(=O)N(C)Cc2ccc(NC(=O)OCc3ccc(Cl)cc3)cc2)n(C)n1] |
| | | | Compound 289: (4-chlorophenyl)methyl N- |
| | | | (4-{[1-(5-ethyl-2-methylpyrazol-3-yl)-N- |
| | | | methylformamido]methyl}phenyl)carbamate. |
| | | | LCMS-ES (POS.) m/z: 441.1 [M + H]⁺. ¹H |
| | | | NMR (400 MHz, DMSO-d₆, temp = 354 K) δ |
| | | | 9.55 (s, 1H), 7.50-7.42 (m, 6H), 7.22-7.13 |
| | | | (m, 2H), 6.24 (s, 1H), 5.16 (s, 2H), 4.58 (s, |
| | | | 2H), 3.77 (s, 3H), 2.94 (s, 3H), 2.60-2.54 (m, |
| | | | 2H), 1.17 (t, J = 7.6 Hz, 3H). |
| 94 | 17.2 | 2-(2- methylpropyl)pyrazole- 3-carboxylic acid | 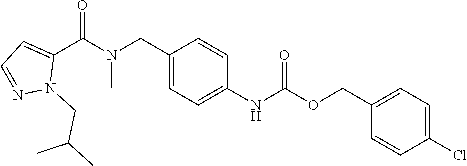 [SMILES: CC(C)Cn1nccc1C(=O)N(C)Cc1ccc(NC(=O)OCc2ccc(Cl)cc2)cc1] |
| | | | Compound 94: (4-chlorophenyl)methyl N- |
| | | | [4-({N-methyl-1-[2-(2- |
| | | | methylpropyl)pyrazol-3- |
| | | | yl]formamido}methyl)phenyl]carbamate. |
| | | | LCMS-ES (POS.) m/z: 455.1 [M + H]+. . ¹H |
| | | | NMR (400 MHz, DMSO-d₆) δ 9.55 (s, 1H), |
| | | | 7.50-7.42 (m, 7H), 7.24-7.19 (m, 2H), 6.44 |
| | | | (s, 1H), 5.16 (s, 2H), 4.59 (s, 2H), 4.02 (d, J = |
| | | | 7.1 Hz, 2H), 2.93 (s, 3H), 2.14-2.01 (m, 1H), |
| | | | 0.82 (d, J = 6.7 Hz, 6H). |
| 90 | 17.2 | 5-methoxy-2- methylpyrazole-3- carboxylic acid | 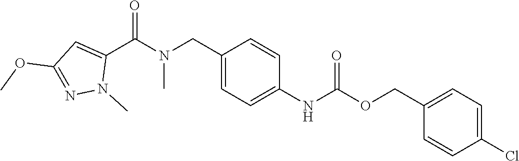 [SMILES: COc1cc(C(=O)N(C)Cc2ccc(NC(=O)OCc3ccc(Cl)cc3)cc2)n(C)n1] |
| | | | Compound 90: (4-chlorophenyl)methyl N- |
| | | | (4-{[1-(5-methoxy-2-methylpyrazol-3-yl)- |
| | | | N- |
| | | | methylformamido]methyl}phenyl)carbamate. |
| | | | LCMS-APCI (POS.) m/z: 443.1 [M + H]⁺. |
| | | | ¹H NMR (400 MHz, DMSO-d₆, temp = 353 K) |
| | | | δ 9.55 (s, 1H), 7.50-7.42 (m, 6H), 7.18 (s, |
| | | | 2H), 5.87 (s, 1H), 5.16 (s, 2H), 4.58 (s, 2H), |
| | | | 3.78 (s, 3H), 3.68 (s, 3H), 2.93 (s, 3H). |
| 253 | 17.2 | 4-methoxy-2- methylpyrazole-3- carboxylic acid | 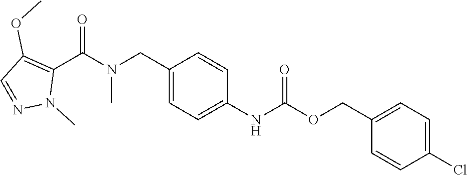 [SMILES: COc1cnn(C)c1C(=O)N(C)Cc1ccc(NC(=O)OCc2ccc(Cl)cc2)cc1] |
| | | | Compound 253: (4-chlorophenyl)methyl N- |
| | | | (4-{[1-(4-methoxy-2-methylpyrazol-3-yl)- |
| | | | N- |
| | | | methylformamido]methyl}phenyl)carbamate. |
| | | | LCMS-ES (POS.) m/z: 443.1 [M + H]⁺. ¹H |
| | | | NMR (400 MHz, DMSO-d₆, temp = 344 K) δ |
| | | | 9.59 (s, 1H), 7.49-6.99 (m, 9H), 5.16 (s, 2H), |
| | | | 3.86-3.68 (m, 6H), 3.20 (s, 2H), 2.89 (s, 3H). |
| 127 | 17.2 | trimethylpyrazole-3- carboxylic acid | 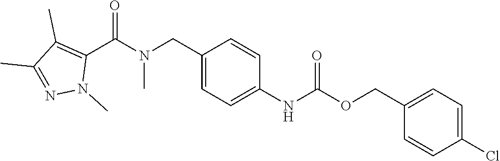 [SMILES: Cc1nn(C)c(C(=O)N(C)Cc2ccc(NC(=O)OCc3ccc(Cl)cc3)cc2)c1C] |
| | | | Compound 127: (4-chlorophenyl)methyl N- |
| | | | (4-{[N-methyl-1-(2,4,5-trimethylpyrazol-3- |
| | | | yl)formamido]methyl}phenyl)carbamate. |
| | | | LCMS-APCI (POS.) m/z: 441.2 [M + H]⁺. ¹H |
| | | | NMR (400 MHz, DMSO-d₆, temp = 343 K) δ |
| | | | 9.61 (s, 1H), 7.53-6.96 (m, 8H), 5.16 (s, 2H), |
| | | | 4.56 (s, 2H), 3.63 (s, 3H), 2.92-2.74 (m, 3H), |
| | | | 2.13-2.05 (m, 3H), 1.89 (s, 3H). |
| 298 | 17.2 | 6- (difluoromethyl)nicotinic acid | 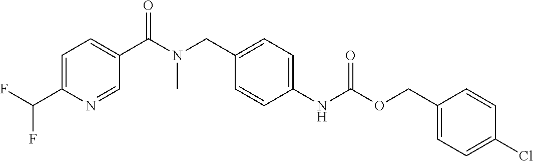 [SMILES: CN(Cc1ccc(NC(=O)OCc2ccc(Cl)cc2)cc1)C(=O)c1ccc(C(F)F)nc1] |
| | | | Compound 298: (4- |
| | | | chlorophenyl)methyl N-[4-({1-[6- |
| | | | (difluoromethyl)pyridin-3-yl]-N- |
| | | | methylformamido}methyl)phenyl]carbamate. |
| | | | LCMS-APCI (POS.) m/z: 460.2 [M + H]⁺. |
| | | | ¹H NMR (400 MHz, 80° C., DMSO-d₆) δ 9.55 |
| | | | (s, 1H), 8.74 (s, 1H), 8.05 (d, J = 7.8 Hz, 1H), |
| | | | 7.74 (d, J = 8.5 Hz, 1H), 7.50-7.40 (m, 6H), |
| | | | 7.21 (s, 2H), 6.94 (t, J = 55.3 Hz, 1H), 5.15 (s, |
| | | | 2H), 4.54 (s, 2H), 2.94-2.80 (m, 3H). |
| 41 | 17.2 | 1H-pyrazole-4- carboxylic acid | 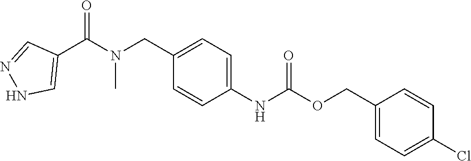 [SMILES: CN(Cc1ccc(NC(=O)OCc2ccc(Cl)cc2)cc1)C(=O)c1cn[nH]c1] |
| | | | Compound 41: (4-chlorophenyl)methyl N- |
| | | | {4-[(N-methyl-1-1H-pyrazol-4- |
| | | | ylformamido)methyl]phenyl}carbamate. |
| | | | LCMS-APCI (POS.) m/z: 399.1 [M + H]⁺. ¹H |
| | | | NMR (400 MHz, DMSO-d₆) δ 13.10 (bs, 1H), |
| | | | 9.78 (s, 1H), 7.95 (bs, 2H), 7.51-7.36 (m, |
| | | | 6H), 7.17 (s, 2H), 5.13 (s, 2H), 4.59 (s, 2H), |
| | | | 3.12-2.81 (m, 3H). |
| 49 | 17.2 | 1H-pyrazole-3- carboxylic acid | 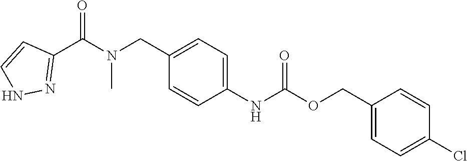 [SMILES: CN(Cc1ccc(NC(=O)OCc2ccc(Cl)cc2)cc1)C(=O)c1cc[nH]n1] |
| | | | Compound 49: (4-chlorophenyl)methyl N- |
| | | | {4-[(N-methyl-1-1H-pyrazol-3- |
| | | | ylformamido)methyl]phenyl}carbamate. |
| | | | LCMS-APCI (POS.) m/z: 399.1 [M + H]⁺. ¹H |
| | | | NMR (400 MHz, 80° C., DMSO-d₆) δ 12.95 (s, |
| | | | 1H), 9.50 (s, 1H), 7.73 (s, 1H), 7.48-7.38 (m, |
| | | | 6H), 7.19 (d, J = 7.8 Hz, 2H), 6.58 (s, 1H), |
| | | | 5.15 (s, 2H), 4.67 (bs, 2H), 3.05 (s, 3H). |
| 69 | 17.2 | 5-fluoro-1H-pyrazole-4- carboxylic acid | 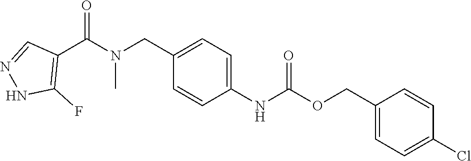 [SMILES: CN(Cc1ccc(NC(=O)OCc2ccc(Cl)cc2)cc1)C(=O)c1cn[nH]c1F] |
| | | | Compound 69: (4-chlorophenyl)methyl N- |
| | | | (4-{[1-(3-fluoro-2H-pyrazol-4-yl)-N- |
| | | | methylformamido]methyl}phenyl)carbamate. |
| | | | LCMS-APCI (POS.) m/z: 417.3 [M + H]⁺. |
| | | | ¹H NMR (400 MHz, 80° C., DMSO-d₆) δ 12.62 |
| | | | (bs, 1H), 9.53 (s, 1H), 7.90 (s, 1H), 7.48-7.41 |
| | | | (m, 6H), 7.16 (d, J = 8.2 Hz, 2H), 5.15 (s, 2H), |
| | | | 4.55 (s, 2H), 2.92 (s, 3H). |
| 101 | 16.2 | 1-ethyl-1H-pyrazole-5- carboxylic acid | 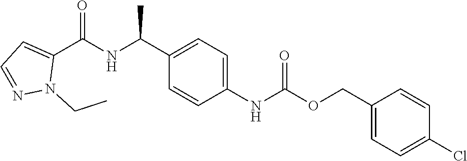 [SMILES: CCn1nccc1C(=O)N[C@@H](C)c1ccc(NC(=O)OCc2ccc(Cl)cc2)cc1] |
| | | | Compound 101: (4-chlorophenyl)methyl N- |
| | | | {4-[(1S)-1-[(2-ethylpyrazol-3- |
| | | | yl)formamido]ethyl]phenyl}carbamate. |
| | | | LCMS-APCI (POS.) m/z: 427.1 [M + H]⁺. ¹H |
| | | | NMR (400 MHz, DMSO-d₆) δ 9.74 (s, 1H), |
| | | | 8.71 (d, J = 8.1 Hz, 1H), 7.50-7.42 (m, 5H), |
| | | | 7.40 (d, J = 8.1 Hz, 2H), 7.27 (d, J = 8.3 Hz, |
| | | | 2H), 6.92 (s, 1H), 5.13 (s, 2H), 5.07 (p, J = |
| | | | 7.9 Hz, 1H), 4.44 (q, J = 7.9 Hz, 2H), 1.47-1.43 |
| | | | (d, J = 5.3 Hz, 3H), 1.25 (t, J = 6.3 Hz, 3H). |
| 104 | 17.2 | 6-methylnicotinic acid | 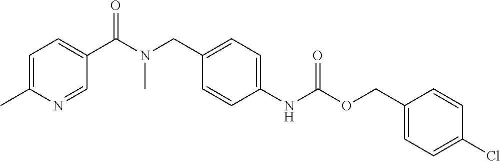 [SMILES: Cc1ccc(C(=O)N(C)Cc2ccc(NC(=O)OCc3ccc(Cl)cc3)cc2)cn1] |
| | | | Compound 104: (4-chlorophenyl)methyl N- |
| | | | (4-{[N-methyl-1-(6-methylpyridin-3- |
| | | | yl)formamido]methyl}phenyl)carbamate. |
| | | | LCMS-APCI (POS.) m/z: 424.1 [M + H]⁺. ¹H |
| | | | NMR (400 MHz, 80° C., DMSO-d₆) δ 9.53 (s, |
| | | | 1H), 8.50 (s, 1H), 7.72 (d, J = 8.1 Hz, 1H), |
| | | | 7.49-7.41 (m, 6H), 7.29 (d, J = 8.2 Hz, 1H), |
| | | | 7.22-7.15 (m, 2H), 5.15 (s, 2H), 4.53 (s, 2H), |
| | | | 2.88 (s, 3H), 2.49 (s, 3H). |
| 105 | 16.2 | isonicotinic acid | 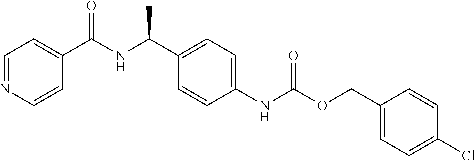 [SMILES: C[C@H](NC(=O)c1ccncc1)c1ccc(NC(=O)OCc2ccc(Cl)cc2)cc1] |
| | | | Compound 105: (4-chlorophenyl)methyl N- |
| | | | {4-[(1S)-1-(pyridin-4- |
| | | | ylformamido)ethyl]phenyl}carbamate. |
| | | | LCMS-APCI (POS.) m/z: 410.1 [M + H]⁺. ¹H |
| | | | NMR (400 MHz, DMSO-d₆) δ 9.74 (s, 1H), |
| | | | 9.02 (d, J = 8.0 Hz, 1H), 8.71 (s, 2H), 7.77 (s, |
| | | | 2H), 7.49-7.43 (m, 4H), 7.40 (d, J = 8.1 Hz, |
| | | | 2H), 7.30 (d, J = 8.3 Hz, 2H), 5.17-5.06 (m, |
| | | | 3H), 1.46 (d, J = 7.0 Hz, 3H). |
| 118 | 16.2 | nicotinic acid | 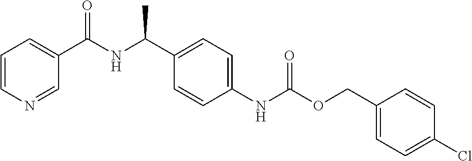 [SMILES: C[C@H](NC(=O)c1cccnc1)c1ccc(NC(=O)OCc2ccc(Cl)cc2)cc1] |
| | | | Compound 118: (4-chlorophenyl)methyl N- |
| | | | {4-[(1S)-1-(pyridin-3- |
| | | | ylformamido)ethyl]phenyl}carbamate. |
| | | | LCMS-APCI (POS.) m/z: 410.1 [M + H]⁺. ¹H |
| | | | NMR (400 MHz, DMSO-d₆) δ 9.74 (s, 1H), |
| | | | 9.02 (s, 1H), 8.94 (d, J = 8.1 Hz, 1H), 8.70 |
| | | | (d, J = 4.6 Hz, 1H), 8.20 (d, J = 8.0 Hz, 1H), |
| | | | 7.54-7.48 (m, 1H), 7.47-7.43 (m, 4H), 7.40 |
| | | | (d, J = 8.4 Hz, 2H), 7.30 (d, J = 8.3 Hz, 2H), |
| | | | 5.17-5.07 (m, 3H), 1.46 (d, J = 7.0 Hz, 3H). |
| 130 | 17.2 | oxazole-5-carboxylic acid | 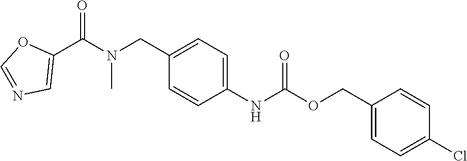 [SMILES: CN(Cc1ccc(NC(=O)OCc2ccc(Cl)cc2)cc1)C(=O)c1cnco1] |
| | | | Compound 130: (4-chlorophenyl)methyl N- |
| | | | (4-[(N-methyl-1-1,3-oxazol-5- |
| | | | ylformamido)methyl]phenyl}carbamate. |
| | | | LCMS-APCI (POS.) m/z: 400.1 [M + H]⁺. ¹H |
| | | | NMR (400 MHz, 80° C., DMSO-d₆) δ 9.53 (s, |
| | | | 1H), 8.45 (s, 1H), 7.65 (s, 1H), 7.48-7.40 (m, |
| | | | 6H), 7.19 (d, J = 8.2 Hz, 2H), 5.15 (s, 2H), |
| | | | 4.64 (s, 2H), 3.05 (s, 3H). |
| 139 | 16.2 | 1,3-dimethyl-1H- pyrazole-5-carboxylic acid | 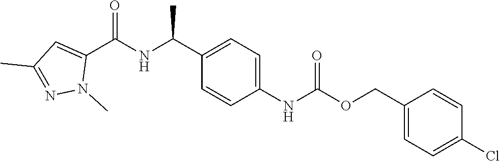 [SMILES: Cc1cc(C(=O)N[C@@H](C)c2ccc(NC(=O)OCc3ccc(Cl)cc3)cc2)n(C)n1] |
| | | | Compound 139: (4-chlorophenyl)methyl N- |
| | | | {4-[(1S)-1-[(2,5-dimethylpyrazol-3- |
| | | | yl)formamido]ethyl]phenyl}carbamate. |
| | | | LCMS-APCI (POS.) m/z: 427.4 [M + H]⁺. ¹H |
| | | | NMR (400 MHz, DMSO-d₆) δ 9.73 (s, 1H), |
| | | | 8.62 (d, J = 8.2 Hz, 1H), 7.49-7.43 (m, 4H), |
| | | | 7.39 (d, J = 8.2 Hz, 2H), 7.26 (d, J = 8.4 Hz, |
| | | | 2H), 6.71 (s, 1H), 5.13 (s, 2H), 5.09-4.99 (m, |
| | | | 1H), 3.91 (s, 3H), 2.15 (s, 3H), 1.41 (d, J = |
| | | | 7.0 Hz, 3H). |
| 155 | 17.2 | 4-methyloxazole-2- carboxylic acid | 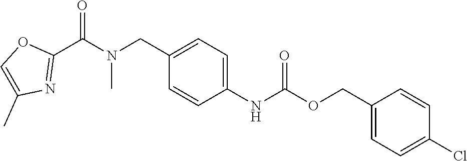 [SMILES: Cc1coc(C(=O)N(C)Cc2ccc(NC(=O)OCc3ccc(Cl)cc3)cc2)n1] |
| | | | Compound 155: (4-chlorophenyl)methyl N- |
| | | | (4-{[N-methyl-1-(4-methyl-1,3-oxazol-2- |
| | | | yl)formamido]methyl}phenyl)carbamate. |
| | | | LCMS-APCI (POS.) m/z: 414.2 [M + H]⁺. ¹H |
| | | | NMR (400 MHz, DMSO-d₆) δ 9.82 (s, 1H), |
| | | | 8.00 (s, 1H), 7.49-7.41 (m, 6H), 7.20 (dd, J = |
| | | | 13.3, 8.3 Hz, 2H), 5.13 (s, 2H), 4.77 (d, J = |
| | | | 143.0 Hz, 2H), 3.04 (d, J = 141.6 Hz, 3H), |
| | | | 2.15 (d, J = 8.6 Hz, 3H). |
| 160 | 17.2 | 3-methylisoxazole-5- carboxylic acid | 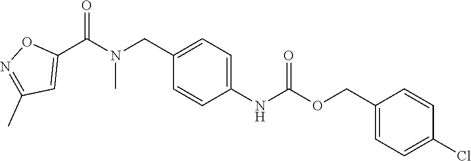 [SMILES: Cc1cc(C(=O)N(C)Cc2ccc(NC(=O)OCc3ccc(Cl)cc3)cc2)on1] |
| | | | Compound 160: (4-chlorophenyl)methyl N- |
| | | | (4-{[N-methyl-1-(3-methyl-1,2-oxazol-5- |
| | | | yl)formamido]methyl}phenyl)carbamate. |
| | | | LCMS-APCI (POS.) m/z: 414.1 [M+H]+. 1H |
| | | | NMR (400 MHz, 80 ° C., DMSO-d6) 8 9.56 (s, |
| | | | 1H), 7.50-7.41 (m, 6H), 7.19 (bs, 2H), 6.75 |
| | | | (bs, 1H), 5.15 (s, 2H), 4.59 (s, 2H), 2.98 (bs, |
| | | | 3H), 2.29 (s, 3H). |
| 182 | 17.2 | 2,4-dimethyloxazole-5- carboxylic acid | 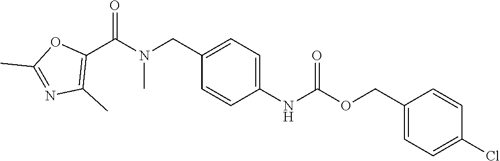 [SMILES: Cc1nc(C)c(C(=O)N(C)Cc2ccc(NC(=O)OCc3ccc(Cl)cc3)cc2)o1] |
| | | | Compound 182: (4-chlorophenyl)methyl N- |
| | | | (4-{[1-(2,4-dimethyl-1,3-oxazol-5-yl)-N- |
| | | | methylformamido]methyl}phenyl)carbamate. |
| | | | LCMS-APCI (POS.) m/z: 428.2 [M + H]⁺. |
| | | | ¹H NMR (400 MHz, 80° C., DMSO-d₆) δ 9.53 |
| | | | (s, 1H), 7.47-7.40 (m, 6H), 7.18 (d, J = 7.7 Hz, |
| | | | 2H), 5.15 (s, 2H), 4.58 (s, 2H), 2.96 (s, |
| | | | 3H), 2.38 (s, 3H), 2.24 (s, 3H). |
| 190 | 17.2 | 3,4-dimethylisoxazole- 5-carboxylic acid | 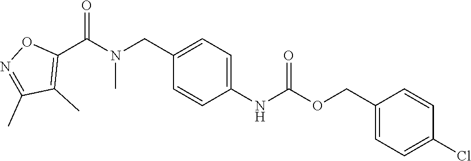 [SMILES: Cc1noc(C(=O)N(C)Cc2ccc(NC(=O)OCc3ccc(Cl)cc3)cc2)c1C] |
| | | | Compound 190: (4-chlorophenyl)methyl N- |
| | | | (4-{[1-(3,4-dimethyl-1,2-oxazol-5-yl)-N- |
| | | | methylformamido]methyl}phenyl)carbamate. |
| | | | LCMS-APCI (POS.) m/z: 428.2 [M + H]⁺. |
| | | | ¹H NMR (400 MHz, 80° C., DMSO-d₆) δ 9.55 |
| | | | (s, 1H), 7.48-7.41 (m, 6H), 7.16 (d, J = 6.4 Hz, |
| | | | 2H), 5.15 (s, 2H), 4.52 (s, 2H), 2.85 (s, |
| | | | 3H), 2.35 (s, 3H), 2.17 (s, 3H). |
| 197 | 17.2 | 3-methyl-1H-pyrazole- 4-carboxylic acid | 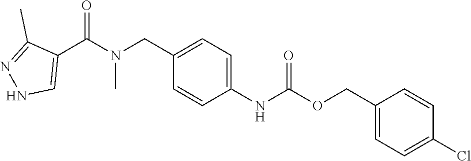 [SMILES: Cc1n[nH]cc1C(=O)N(C)Cc1ccc(NC(=O)OCc2ccc(Cl)cc2)cc1] |
| | | | Compound 197: (4-chlorophenyl)methyl N- |
| | | | (4-{[N-methyl-1-(3-methyl-1H-pyrazol-4- |
| | | | yl)formamido]methyl}phenyl)carbamate. |
| | | | LCMS-APCI (POS.) m/z: 413.3 [M + H]+. ¹H |
| | | | NMR (400 MHz, 80° C., DMSO-d₆) δ 12.59 |
| | | | (bs, 1H), 9.51 (s, 1H), 7.58 (s, 1H), 7.47- |
| | | | 7.41 (m, 6H), 7.16 (d, J = 8.3 Hz, 2H), 5.15 |
| | | | (s, 2H), 4.56 (s, 2H), 2.92 (s, 3H), 2.29 (s, |
| | | | 3H). |
| 266 | 16.2 | cyclopropanecarboxylic acid | 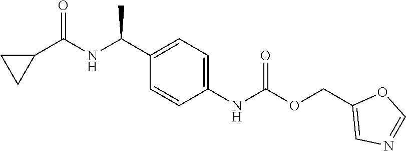 [SMILES: C[C@H](NC(=O)C1CC1)c1ccc(NC(=O)OCc2cnco2)cc1] |
| | | | Compound 266: 1,3-oxazol-5-ylmethyl N- |
| | | | {4-[(1S)-1- |
| | | | (cyclopropylformamido)ethyl]phenyl}carbamate. |
| | | | LCMS-APCI (POS.) m/z: 330.2 |
| | | | [M + H]⁺. ¹H NMR (400 MHz, DMSO-d₆) δ |
| | | | 9.76 (s, 1H), 8.46-8.37 (m, 2H), 7.38 (d, J = |
| | | | 8.1 Hz, 2H), 7.31 (s, 1H), 7.22 (d, J = 8.1 Hz, |
| | | | 2H), 5.22 (s, 2H), 4.87 (p, J = 7.2 Hz, 1H), |
| | | | 1.59 (p, J = 6.3 Hz, 1H), 1.32 (d, J = 6.9 Hz, |
| | | | 3H), 0.70-0.57 (m, 4H). |
| 75 | 16.2 | 3,3- difluorocyclobutane-1- carboxylic acid | 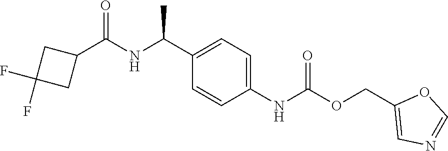 [SMILES: C[C@H](NC(=O)C1CC(F)(F)C1)c1ccc(NC(=O)OCc2cnco2)cc1] |
| | | | Compound 75: 1,3-oxazol-5-ylmethyl N- |
| | | | {4-[(1S)-1-[(3,3- |
| | | | difluorocyclobutyl)formamido]ethyl]phenyl} |
| | | | carbamate. LCMS-APCI (POS.) m/z: 380.2 |
| | | | [M + H]⁺. ¹H NMR (400 MHz, DMSO-d₆) δ |
| | | | 9.76 (s, 1H), 8.42 (s, 1H), 8.38 (d, J = 7.8 Hz, |
| | | | 1H), 7.37 (d, J = 8.1 Hz, 2H), 7.30 (s, 1H), |
| | | | 7.20 (d, J = 8.0 Hz, 2H), 5.21 (s, 2H), 4.86 |
| | | | (p, J = 6.9 Hz, 1H), 2.90 (p, J = 8.9 Hz, 1H), |
| | | | 2.74-2.57 (m, 4H), 1.31 (d, J = 6.9 Hz, 3H). |
| 39 | 16.2 | 4-fluorobenzoic acid | 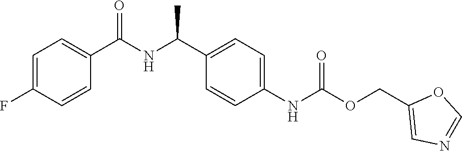 [SMILES: C[C@H](NC(=O)c1ccc(F)cc1)c1ccc(NC(=O)OCc2cnco2)cc1] |
| | | | Compound 39: 1,3-oxazol-5-ylmethyl N-{4- |
| | | | [(1S)-1-[(4- |
| | | | fluorophenyl)formamido]ethyl]phenyl} |
| | | | carbamate. LCMS-APCI (POS.) m/z: 384.2 |
| | | | [M + H]⁺. ¹H NMR (400 MHz, DMSO-d₆) δ |
| | | | 9.76 (s, 1H), 8.75 (d, J = 8.2 Hz, 1H), 8.41 (s, |
| | | | 1H), 7.99-7.86 (m, 2H), 7.38 (d, J = 8.2 Hz, |
| | | | 2H), 7.33-7.22 (m, 5H), 5.20 (s, 2H), 5.09 |
| | | | (p, J = 7.4 Hz, 1H), 1.44 (d, J = 7.0 Hz, 3H). |
| 97 | 16.2 | 3,3- difluorocyclopentane-1- carboxylic acid | 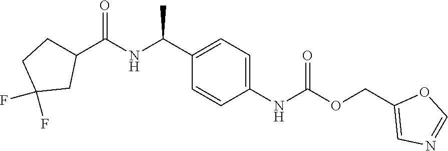 [SMILES: C[C@H](NC(=O)C1CCC(F)(F)C1)c1ccc(NC(=O)OCc2cnco2)cc1] |
| | | | Compound 97: 1,3-oxazol-5-ylmethyl N-{4- |
| | | | [(1S)-1-[(3,3- |
| | | | difluorocyclopentyl)formamido]ethyl]phenyl} |
| | | | carbamate. LCMS-APCI (POS.) m/z: |
| | | | 394.2 [M + H]⁺. ¹H NMR (400 MHz, DMSO- |
| | | | d₆) δ 9.76 (s, 1H), 8.42 (s, 1H), 8.33-8.26 (m, |
| | | | 1H), 7.37 (d, J = 8.2 Hz, 2H), 7.30 (s, 1H), |
| | | | 7.20 (d, J = 8.1 Hz, 2H), 5.21 (s, 2H), 4.83 |
| | | | (p, J = 7.5 Hz, 1H), 2.90 (p, J = 9.0 Hz, 1H), |
| | | | 2.31-1.91 (m, 5H), 1.88-1.66 (m, 1H), 1.31 |
| | | | (d, J = 6.9 Hz, 3H). |
| 22 | 16.2 | 6,6- difluorospiro[3.3]heptane- 2-carboxylic acid | 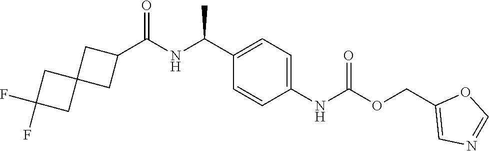 [SMILES: C[C@H](NC(=O)C1CC2(C1)CC(F)(F)C2)c1ccc(NC(=O)OCc2cnco2)cc1] |
| | | | Compound 22: 1,3-oxazol-5-ylmethyl N-{4- |
| | | | [(1S)-1-({6,6-difluorospiro[3.3]heptan-2- |
| | | | yl}formamido)ethyl]phenyl}carbamate. |
| | | | LCMS-APCI (POS.) m/z: 420.2 [M + H]⁺. ¹H |
| | | | NMR (400 MHz, DMSO-d₆) δ 9.75 (s, 1H), |
| | | | 8.41 (s, 1H), 8.07 (d, J = 8.0 Hz, 1H), 7.36 |
| | | | (d, J = 8.1 Hz, 2H), 7.30 (s, 1H), 7.18 (d, J = |
| | | | 8.1 Hz, 2H), 5.20 (s, 2H), 4.83 (p, J = 7.0, |
| | | | 6.5 Hz, 1H), 2.95 (p, J = 8.6 Hz, 1H), 2.62 (t, J = |
| | | | 12.7 Hz, 2H), 2.49-2.42 (m, 2H), 2.30-2.21 |
| | | | (m, 1H), 2.20-2.10 (m, 3H), 1.28 (d, J = 6.9 Hz, |
| | | | 3H). |
| 230 | 16.2 | (1R,3r,5S)-6,6- difluorobicyclo[3.1.0] hexane-3-carboxylic acid | 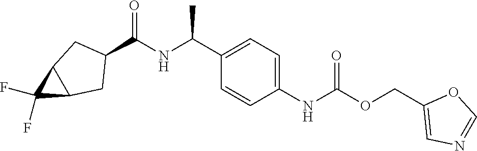 [SMILES: C[C@H](NC(=O)[C@H]1C[C@@H]2[C@H](C1)C2(F)F)c1ccc(NC(=O)OCc2cnco2)cc1] |
| | | | Compound 230: 1,3-oxazol-5-ylmethyl N- |
| | | | {4-[(1S)-1-{[(1R,3r,5S)-6,6- |
| | | | difluorobicyclo[3.1.0]hexan-3- |
| | | | yl]formamido}ethyl]phenyl}carbamate. |
| | | | LCMS-ESI (POS.) m/z: 406.1 [M + H]⁺. ¹H |
| | | | NMR (400 MHz, DMSO-d₆) δ 9.75 (s, 1H), |
| | | | 8.42 (s, 1H), 8.08 (d, J = 8.0 Hz, 1H), 7.36 |
| | | | (d, J = 8.1 Hz, 2H), 7.30 (s, 1H), 7.17 (d, J = |
| | | | 8.1 Hz, 2H), 5.20 (s, 2H), 4.82 (p, J = 7.4 Hz, |
| | | | 1H), 3.13 (p, J = 9.6 Hz, 1H), 2.18 (d, J = |
| | | | 12.3 Hz, 4H), 1.86 (t, J = 10.5 Hz, 1H), 1.78z |
| | | | (t, J = 10.6 Hz, 1H), 1.29 (d, J = 6.9 Hz, 3H). |
| 68 | 16.2 | (1R,3s,5S)-6,6- difluorobicyclo[3.1.0] hexane-3-carboxylic acid | 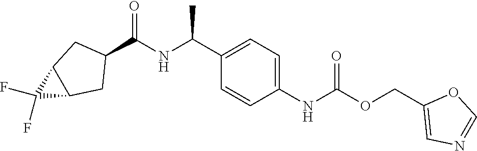 [SMILES: C[C@H](NC(=O)[C@@H]1C[C@@H]2[C@H](C1)C2(F)F)c1ccc(NC(=O)OCc2cnco2)cc1] |
| | | | Compound 68: 1,3-oxazol-5-ylmethyl N-{4- |
| | | | [(1S)-1-{[(1R,3s,5S)-6,6- |
| | | | difluorobicyclo[3.1.0]hexan-3- |
| | | | yl]formamido}ethyl]phenyl}carbamate. |
| | | | LCMS-ESI (POS.) m/z: 406.1 [M+H]+. 1H |
| | | | NMR (400 MHz, DMSO-d6) 8 9.75 (s, 1H), |
| | | | 8.41 (s, 1H), 8.30 (d, J = 7.8 Hz, 1H), 7.36 |
| | | | (d, J = 7.6 Hz, 2H), 7.30 (s, 1H), 7.18 (d, J = |
| | | | 8.5 Hz, 2H), 5.20 (s, 2H), 4.82 (p, J = 7.2 Hz, |
| | | | 1H), 2.70-2.57 (m, 1H), 2.18-1.96 (m, 6H), |
| | | | 1.28 (d, J = 6.9 Hz, 3H). |

### Urea Synthesis

### Example N

### Preparation of (4-chlorophenyl)methyl N-{4-[1(morpholine-4-carbonylamino)methyl]phenyl}carbamate (Compound 270)

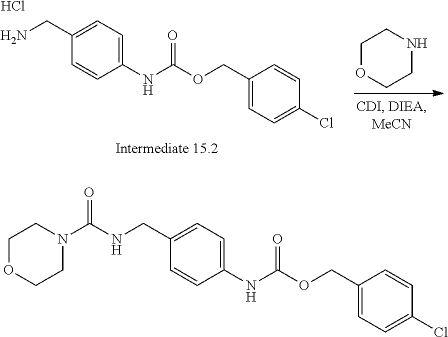

To a solution of (4-chlorophenyl)methyl N-[4-(aminomethyl)phenyl]carbamate hydrochloride (100 mg, 0.31 mmol) and N,N-diisopropylethylamine (0.21 mL, 1.22 mmol) in acetonitrile (1 mL) and DCM (1 mL) was added morpholine (0.14 mL, 1.83 mmol), carbonyldiimidazole (178.4 mg, 1.1 mmol), the mixture was stirred at 24° C. for 15 h, concentrated and purified by reverse phase prep HPLC using a gradient of 3-40% water/acetonitrile with 0.1% formic acid to yield product (4-chlorophenyl)methyl N-{4-[(morpholine-4-carbonylamino)methyl]phenyl}carbamate (61.0 mg, 0.15 mmol, 49% yield). LCMS-APCI (POS.) m/z: 404.2 [M+H]⁺. ¹H NMR (400 MHz, DMSO-d₆) δ 9.71 (s, 1H), 7.49-7.42 (m, 4H), 7.37 (d, J=8.1 Hz, 2H), 7.16 (d, J=8.0 Hz, 2H), 7.02 (t, J=6.2 Hz, 1H), 5.13 (s, 2H), 4.16 (d, J=5.6 Hz, 2H), 3.56-3.51 (m, 4H), 3.30-3.24 (m, 4H).

Compounds in the following table were prepared in a similar manner as Compound 270, using the intermediate and amine as listed.

| Co | | | |
|---|---|---|---|
| # | Intermediate | Amine | Structure, Name and Data |
| 184 | 15.2 | 4- methoxypiperidine | 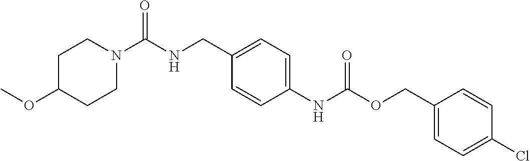 [SMILES: COC1CCN(C(=O)NCc2ccc(NC(=O)OCc3ccc(Cl)cc3)cc2)CC1] |
| | | | Compound 184: (4 |
| | | | chlorophenyl)methyl N-{4-[(4- |
| | | | methoxypiperidine-1- |
| | | | carbonylamino)methyl]phenyl}carbamate. |
| | | | LCMS-APCI (POS.) m/z: 432.2 [M + H]⁺. ¹H |
| | | | NMR (400 MHz, DMSO-d₆) δ 9.70 (s, 1H), |
| | | | 7.49-7.42 (m, 4H), 7.37 (d, J = 8.1 Hz, 2H), |
| | | | 7.14 (d, J = 8.0 Hz, 2H), 6.98 (t, J = 5.2 Hz, |
| | | | 1H), 5.13 (s, 2H), 4.14 (d, J = 5.6 Hz, 2H), |
| | | | 3.66 (d, J = 13.5 Hz, 2H), 3.31-3.36 (m, |
| | | | 1H), 3.24 (s, 3H), 2.95 (dd, J = 13.2, 9.8 Hz, |
| | | | 2H), 1.86-1.69 (m, 2H), 1.35-1.18 (m, 2H). |
| 96 | 16.2 | azetidine | 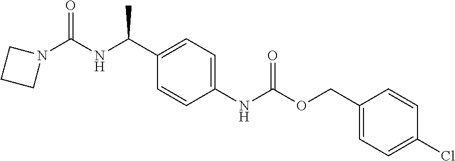 [SMILES: C[C@H](NC(=O)N1CCC1)c1ccc(NC(=O)OCc2ccc(Cl)cc2)cc1] |
| | | | Compound 96: 4-chlorophenyl)methyl N- |
| | | | {4-[(1S)-1-(azetidine-1- |
| | | | carbonylamino)ethyl]phenyl}carbamate. |
| | | | LCMS-APCI (POS.) m/z: 388.1 [M + H]⁺. 1H |
| | | | NMR (400 MHz, DMSO-d6) δ 9.70 (s, 1H), |
| | | | 7.46 (d, J = 1.9 Hz, 4H), 7.37 (d, J = 8.2 Hz, |
| | | | 2H), 7.21 (d, J = 8.2 Hz, 2H), 6.51-6.47 (m, |
| | | | 1H), 5.13 (s, 2H), 4.70 (t, J = 7.5 Hz, 1H), |
| | | | 3.78 (t, J = 7.8 Hz, 4H), 2.14-2.06 (m, 2H), |
| | | | 1.30 (d, J = 7.3 Hz, 3H). |
| 144 | 16.2 | 3-methylazetidine | 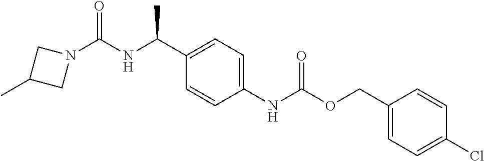 [SMILES: CC1CN(C(=O)N[C@@H](C)c2ccc(NC(=O)OCc3ccc(Cl)cc3)cc2)C1] |
| | | | Compound 144: (4- |
| | | | chlorophenyl)methyl N-{4-[(1S)-1-(3- |
| | | | methylazetidine-1- |
| | | | carbonylamino)ethyl]phenyl}carbamate. |
| | | | LCMS-APCI (POS.) m/z: 402.1 [M + H]⁺. 1H |
| | | | NMR (400 MHz, Methanol-d4) δ 7.50-7.35 |
| | | | (m, 6H), 7.33-7.22 (m, 2H), 5.17 (s, 2H), |
| | | | 4.60 (s, 1H), 4.06 (t, J = 8.1 Hz, 2H), 3.50 (t, |
| | | | J = 6.6 Hz, 2H), 2.73-2.64 (m, 1H), 1.43 (d, |
| | | | J = 6.9 Hz, 3H), 1.25 (d, J = 6.8 Hz, 3H). Two |
| | | | N—H hydrogens. Two hydrogens exchange |
| | | | with solvent. |
| 170 | 16.2 | 3- methoxyazetidine | 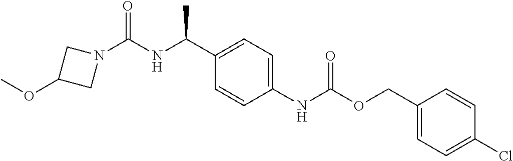 [SMILES: COC1CN(C(=O)N[C@@H](C)c2ccc(NC(=O)OCc3ccc(Cl)cc3)cc2)C1] |
| | | | Compound 170: (4-chlorophenyl)methyl |
| | | | N-{4-[(1S)-1-(3-methoxyazetidine-1- |
| | | | carbonylamino)ethyl]phenyl}carbamate. |
| | | | LCMS-APCI (POS.) m/z: 418.2 [M + H]⁺. 1H |
| | | | NMR (400 MHz, DMSO-d6) δ 9.71 (s, 1H), |
| | | | 7.46 (d, J = 2.5 Hz, 4H), 7.37 (d, J = 7.5 Hz, |
| | | | 2H), 7.21 (d, J = 8.6 Hz, 2H), 6.62 (d, J = 8.2 |
| | | | Hz, 1H), 5.14 (s, 2H), 4.70 (t, J = 7.9 Hz, 1H), |
| | | | 4.11 (s, 1H), 3.96 (s, 2H), 3.64-3.56 (m, 2H), |
| | | | 3.18 (d, J = 2.6 Hz, 3H), 1.31 (d, J = 7.2 Hz, |
| | | | 3H). |
| 265 | 15.2 | azetidine | 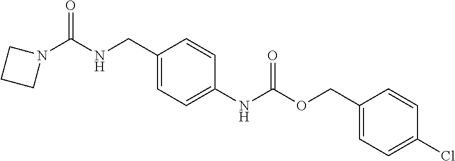 [SMILES: O=C(Nc1ccc(CNC(=O)N2CCC2)cc1)OCc1ccc(Cl)cc1] |
| | | | Compound 265: (4- |
| | | | chlorophenyl)methyl N-{4-[(azetidine-1- |
| | | | carbonylamino)methyl]phenyl}carbamate. |
| | | | LCMS-ES (Pos) m/z: 374 [M + H]⁺. ¹H NMR |
| | | | (400 MHz, DMSO-d₆) δ 9.75 (s ,1H), 7.46 (s, |
| | | | 4H), 7.38 (d, J = 8.2 Hz, 2H), 7.15 (d, J = 8.1 |
| | | | Hz, 2H), 6.71 (s, 1H), 5.13 (s, 2H), 4.10 (d, J = |
| | | | 5.1 Hz, 2H), 3.79 (t, J = 7.3 Hz, 3H), 2.11 |
| | | | (dd, J = 15.4, 7.7 Hz, 3H). |
| 286 | 15.2 | piperidine | 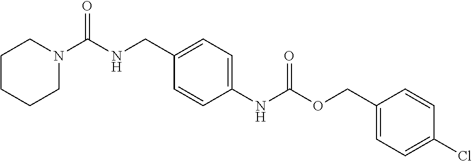 [SMILES: O=C(Nc1ccc(CNC(=O)N2CCCCC2)cc1)OCc1ccc(Cl)cc1] |
| | | | Compound 286: (4- |
| | | | chlorophenyl)methyl N-{4-[(piperidine-1- |
| | | | carbonylamino)methyl]phenyl}carbamate. |
| | | | LCMS-ES (Pos) m/z: 402 [M + H]⁺. ¹H NMR |
| | | | (400 MHz, DMSO-d₆) δ 9.72 (s, 1H), 7.52- |
| | | | 7.44 (m, 4H), 7.37 (d, J = 8.1 Hz, 2H), 7.15 |
| | | | (d, J = 8.2 Hz, 2H), 6.92 (t, J = 6.0 Hz, 1H), |
| | | | 5.13 (s, 2H), 4.15 (d, J = 5.7 Hz, 2H), 3.31- |
| | | | 3.25 (m, 4H), 1.57-1.49 (m, 2H), 1.45-1.37 |
| | | | (m, 4H). |
| 309 | 15.2 | 3,3- Difluoropiperdine | 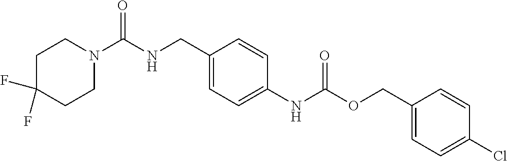 [SMILES: O=C(Nc1ccc(CNC(=O)N2CCC(F)(F)CC2)cc1)OCc1ccc(Cl)cc1] |
| | | | Compound 309: (4- |
| | | | chlorophenyl)methyl N-{4-[(4,4- |
| | | | difluoropiperidine-1- |
| | | | carbonylamino)methyl]phenyl}carbamate. |
| | | | LCMS-ES (Pos) m/z: 438 (M + H). ¹H NMR |
| | | | (400 MHz, DMSO-d₆) δ 9.74 (s, 1H), 7.46 (s, |
| | | | 2H), 7.51-7.35 (m, 4H), 7.24-7.13 (m, 3H), |
| | | | 5.13 (s, 2H), 4.17 (d, J = 5.7 Hz, 2H), 3.34 (s, |
| | | | 3H), 1.89 (dt, J = 14.5, 8.5 Hz, 5H). |
| 258 | 15.2 | tert-butyl piperazine- 1-carboxylate | 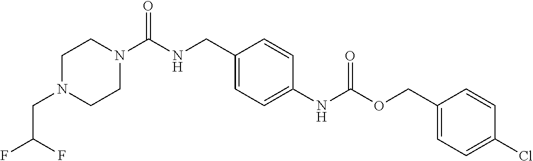 [SMILES: O=C(Nc1ccc(CNC(=O)N2CCN(CC(F)F)CC2)cc1)OCc1ccc(Cl)cc1] |
| | | | Compound 258: (4- |
| | | | chlorophenyl)methyl N-(4-{[4-(2,2- |
| | | | difluoroethyl)piperazine-1- |
| | | | carbonylamino]methyl}phenyl)carbamate. |
| | | | LCMS-ES (Pos) m/z: 467 [M + H]⁺. ¹H NMR |
| | | | (400 MHz, DMSO-d₆) δ 9.73 (s, 1H), 7.46 |
| | | | (s, 4H), 7.38 (d, J = 8.1 Hz, 2H), 7.15 (d, J = |
| | | | 8.1 Hz, 2H), 7.02 (t, J = 5.9 Hz, 2H), 6.17 |
| | | | (m, 1H) 5.13 (s, 2H), 4.16 (d, J = 5.6 Hz, |
| | | | 2H), 3.30 (t, J = 4.8 Hz, 3H), 2.73 (td, J = |
| | | | 15.7, 4.4 Hz, 2H), 2.46 (d, J = 5.4 Hz, 4H). |
| 154 | 16.2 | morpholine | 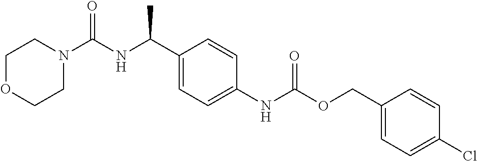 [SMILES: C[C@H](NC(=O)N1CCOCC1)c1ccc(NC(=O)OCc2ccc(Cl)cc2)cc1] |
| | | | Compound 154: 4-chlorobenzyl (S)-(4-(1- |
| | | | (morpholine-4- |
| | | | carboxamido)ethyl)phenyl)carbamate. |
| | | | LCMS-APCI (POS.) m/z: 418.1 [M + H]+. |
| | | | 1H NMR (400 MHz, DMSO-d6) δ 9.69 (s, |
| | | | 1H), 7.45 (d, J = 2.3 Hz, 4H), 7.36 (d, J = 8.3 |
| | | | Hz, 2H), 7.21 (d, J = 8.6 Hz, 2H), 6.71 (d, J = |
| | | | 7.9 Hz, 1H), 5.13 (s, 2H), 4.77 (t, J = 7.4 |
| | | | Hz, 1H), 3.52 (t, J = 4.6 Hz, 4H), 3.26 (t, J = |
| | | | 4.7 Hz, 4H), 1.32 (d, J = 7.1 Hz, 3H). |
| 145 | 16.2 | 4- methylpiperidine | 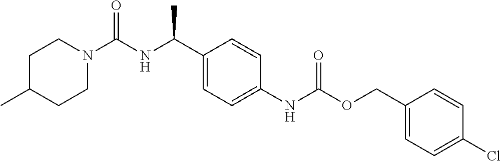 [SMILES: CC1CCN(C(=O)N[C@@H](C)c2ccc(NC(=O)OCc3ccc(Cl)cc3)cc2)CC1] |
| | | | Compound 145: 4-chlorobenzyl (S)-(4-(1- |
| | | | (4-methylpiperidine-1- |
| | | | carboxamido)ethyl)phenyl)carbamate. |
| | | | LCMS-APCI (POS.) m/z: 430.1 [M + H]⁺. |
| | | | 1H NMR (400 MHz, DMSO-d6) δ 9.68 (s, |
| | | | 1H), 7.45 (d, J = 2.2 Hz, 4H), 7.35 (d, J = 8.1 |
| | | | Hz, 2H), 7.20 (d, J = 8.1 Hz, 2H), 6.59 (d, J = |
| | | | 7.9 Hz, 1H), 5.13 (s, 2H), 4.79-4.73 (m, 1H), |
| | | | 3.95 (d, J = 13.2 Hz, 2H), 2.59 (s, 3H), 1.54 |
| | | | (d, J = 13.7 Hz, 2H), 1.31 (d, J = 7.0 Hz, 3H), |
| | | | 0.93 (d, J = 11.8 Hz, 2H), 0.88 (d, J = 6.3 Hz, |
| | | | 3H). |
| 103 | 16.2 | pyrrolidine | 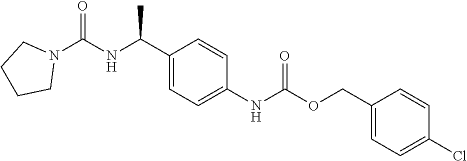 [SMILES: C[C@H](NC(=O)N1CCCC1)c1ccc(NC(=O)OCc2ccc(Cl)cc2)cc1] |
| | | | Compound 103: 4-chlorobenzyl (S)-(4-(1- |
| | | | (pyrrolidine-1- |
| | | | carboxamido)ethyl)phenyl)carbamate. |
| | | | LCMS-APCI (POS.) m/z: 402.1 [M + H]⁺. |
| | | | 1H NMR (400 MHz, DMSO-d6) δ 9.69 (s, |
| | | | 1H), 7.46 (d, J = 2.1 Hz, 4H), 7.36 (d, J = 8.3 |
| | | | Hz, 2H), 7.23 (d, J = 8.4 Hz, 2H), 6.23 (d, J = |
| | | | 8.3 Hz, 1H), 5.13 (s, 2H), 4.78 (t, J = 7.6 |
| | | | Hz, 1H), 3.23 (d, J = 6.2 Hz, 4H), 1.79 (d, J = |
| | | | 6.2 Hz, 4H), 1.33 (d, J = 7.2 Hz, 3H). |
| 72 | 16.2 | 4- methoxypiperidine | 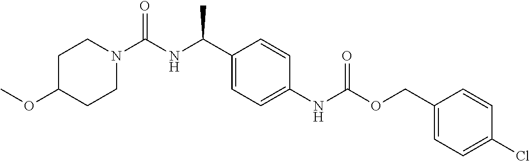 [SMILES: COC1CCN(C(=O)N[C@@H](C)c2ccc(NC(=O)OCc3ccc(Cl)cc3)cc2)CC1] |
| | | | Compound 72: 4-chlorobenzyl (S)-(4-(1- |
| | | | (4-methoxypiperidine-1- |
| | | | carboxamido)ethyl)phenyl)carbamate. |
| | | | LCMS-APCI (POS.) m/z: 446.1 [M + H]⁺. |
| | | | 1H NMR (400 MHz, DMSO-d6) δ 9.69 (s, |
| | | | 1H), 7.45 (d, J = 2.2 Hz, 4H), 7.35 (d, J = 8.2 |
| | | | Hz, 2H), 7.20 (d, J = 8.2 Hz, 2H), 6.67 (d, J = |
| | | | 8.1 Hz, 1H), 5.13 (s, 2H), 4.75 (t, J = 7.3 Hz, |
| | | | 1H), 3.67 (d, J = 13.8 Hz, 2H), 3.24 (s, 3H), |
| | | | 2.93 (t, J = 11.4 Hz, 2H), 1.76 (d, J = 12.1 Hz, |
| | | | 2H), 1.31 (d, J = 7.1 Hz, 3H), 1.25 (d, J = 10.2 |
| | | | Hz, 2H), 1.09 (m, 1H). |
| 112 | 17.2 | azetidine | 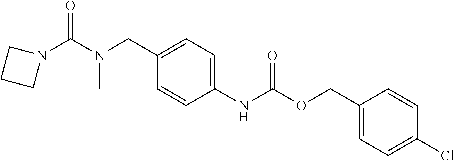 [SMILES: CN(Cc1ccc(NC(=O)OCc2ccc(Cl)cc2)cc1)C(=O)N1CCC1] |
| | | | Compound 112: 4-chlorobenzyl (4-((N- |
| | | | methylazetidine-1- |
| | | | carboxamido)methyl)phenyl)carbamate. |
| | | | LCMS-APCI (POS.) m/z: 388.1 [M + H]⁺. |
| | | | 1H NMR (400 MHz, DMSO-d6) δ 9.76 (s, |
| | | | 1H), 7.45 (s, 4H), 7.41 (d, J = 7.9 Hz, 2H), |
| | | | 7.14 (d, J = 8.0 Hz, 2H), 5.13 (s, 2H), 4.27 (s, |
| | | | 2H), 3.88 (t, J = 7.6 Hz, 4H), 2.64 (s, 3H), |
| | | | 2.17-2.07 (m, 2H). |
| 128 | 17.2 | pyrrolidine | 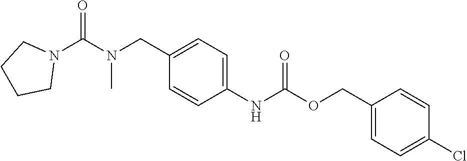 [SMILES: CN(Cc1ccc(NC(=O)OCc2ccc(Cl)cc2)cc1)C(=O)N1CCCC1] |
| | | | Compound 128: 4-chlorobenzyl (4-((N- |
| | | | methylpyrrolidine-1- |
| | | | carboxamido)methyl)phenyl)carbamate. |
| | | | LCMS-APCI (POS.) m/z: 402.1 [M + H]⁺. |
| | | | 1H NMR (400 MHz, DMSO-d6) δ 9.76 (s, |
| | | | 1H), 7.45 (s, 4H), 7.41 (d, J = 8.3 Hz, 2H), |
| | | | 7.18 (d, J = 8.2 Hz, 2H), 5.13 (s, 2H), 4.25 (s, |
| | | | 2H), 3.26 (d, J = 6.5 Hz, 4H), 2.64 (s, 3H), |
| | | | 1.75 (d, J = 6.0 Hz, 4H). |
| 42 | 17.2 | 4-methoxy- piperidine | 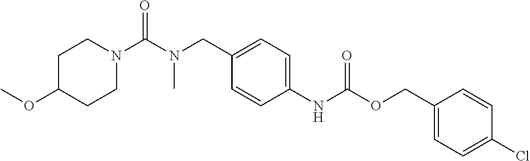 [SMILES: COC1CCN(C(=O)N(C)Cc2ccc(NC(=O)OCc3ccc(Cl)cc3)cc2)CC1] |
| | | | Compound 42: 4-chlorobenzyl (4-((4- |
| | | | methoxy-N-methylpiperidine-1- |
| | | | carboxamido)methyl)phenyl)carbamate. |
| | | | LCMS-APCI (POS.) m/z: 446.1 [M + H]+. |
| | | | 1H NMR (400 MHz, DMSO-d6) δ 9.77 (s, |
| | | | 1H), 7.45 (s, 4H), 7.42 (d, J = 8.2 Hz, 2H), |
| | | | 7.15 (d, J = 8.1 Hz, 2H), 5.13 (s, 2H), 4.22 (s, |
| | | | 2H), 3.45-3.3 (m, 3H), 3.24 (s, 3H), 2.87 (s, |
| | | | 2H), 2.63 (s, 3H), 1.87-1.77 (m, 2H), 1.38 |
| | | | (d, J = 10.6 Hz, 2H). |
| 89 | 18.2 | azetidine | 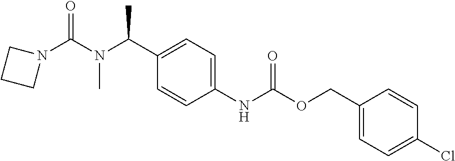 [SMILES: C[C@@H](c1ccc(NC(=O)OCc2ccc(Cl)cc2)cc1)N(C)C(=O)N1CCC1] |
| | | | Compound 89: 4-chlorobenzyl (S)-(4-(1- |
| | | | (N-methylazetidine-1- |
| | | | carboxamido)ethyl)phenyl)carbamate. |
| | | | LCMS-APCI (POS.) m/z: 402.1 [M + H]⁺. |
| | | | 1H NMR (400 MHz, DMSO-d6) δ 9.78 (s, |
| | | | 1H), 7.46 (s, 4H), 7.42 (d, J = 8.5 Hz, 2H), |
| | | | 7.19 (d, J = 8.2 Hz, 2H), 5.23 (d, J = 7.0 Hz, |
| | | | 1H), 5.14 (s, 2H), 3.89 (t, J = 7.7 Hz, 4H), |
| | | | 2.41 (s, 3H), 2.18-2.09 (m, 2H), 1.39 (d, J = |
| | | | 7.1 Hz, 3H). |
| 143 | 18.2 | pyrrolidine | 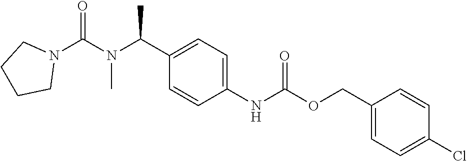 [SMILES: C[C@@H](c1ccc(NC(=O)OCc2ccc(Cl)cc2)cc1)N(C)C(=O)N1CCCC1] |
| | | | Compound 143: 4-chlorobenzyl (S)-(4-(1- |
| | | | (N-methylpyrrolidine-1- |
| | | | carboxamido)ethyl)phenyl)carbamate. |
| | | | LCMS-APCI (POS.) m/z: 416.5 [M + H]⁺. 1H |
| | | | NMR (400 MHz, DMSO-d6) δ 9.77 (s, 1H), |
| | | | 7.46 (s, 4H), 7.41 (d, J = 8.3 Hz, 2H), 7.22 (d, |
| | | | J = 8.1 Hz, 2H), 5.13 (s, 2H), 5.08 (d, J = 7.3 |
| | | | Hz, 1H), 3.26 (d, J = 6.3 Hz, 4H), 2.42 (s, 3H), |
| | | | 1.75 (s, 4H), 1.42 (d, J = 7.4 Hz, 3H). |
| 134 | 18.2 | 4-methoxy- piperidine | 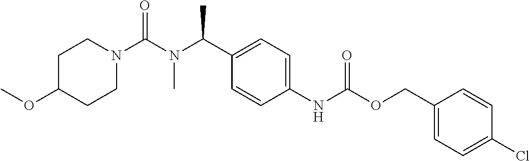 [SMILES: COC1CCN(C(=O)N(C)[C@@H](C)c2ccc(NC(=O)OCc3ccc(Cl)cc3)cc2)CC1] |
| | | | Compound 134: 4-chlorobenzyl (S)-(4-(1- |
| | | | (4-methoxy-N-methylpiperidine-1- |
| | | | carboxamido)ethyl)phenyl)carbamate. |
| | | | LCMS-APCI (POS.) m/z: 460.1 [M + H]⁺. |
| | | | ¹H NMR (400 MHz, DMSO-d6) δ 9.77 (s, |
| | | | 1H), 7.45 (s, 4H), 7.41 (d, J = 7.5 Hz, 2H), |
| | | | 7.19 (d, J = 8.2 Hz, 2H), 5.13 (s, 2H), 5.00 (s, |
| | | | 1H), 3.24 (s, 3H), 2.85 (s, 3H), 2.44 (s, 3H), |
| | | | 1.82 (d, 3H), 1.42 (d, J = 7.1 Hz, 4H), 1.37 |
| | | | (m, 2H), 1.23 (s, 1H). |

### Ester Hydrolysis and Amide Synthesis

### Example O

### Preparation of (4-chlorophenyl)methyl N-{4-[2-(4-hydroxy-4-methylpiperidin-1-yl)-2-oxoethyl]phenyl}carbamate (Compound 116)

### Step 1: Preparation of [4-({[(4-chlorophenyl)methoxy]carbonyl}amino)phenyl]acetic acid

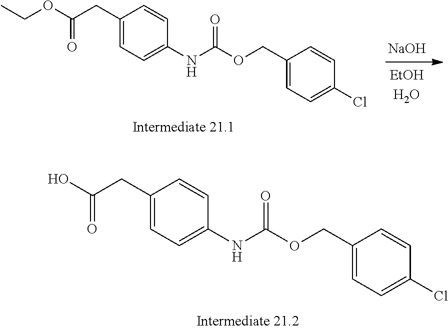 [SMILES: "EtOHH2O" ; CCOC(=O)Cc1ccc(NC(=O)OCc2ccc(Cl)cc2)cc1 ; "NaOH" ; O=C(O)Cc1ccc(NC(=O)OCc2ccc(Cl)cc2)cc1]

A mixture of ethyl 2-[4-({[(4-chlorophenyl)methoxy]carbonyl}amino)phenyl]acetate, 150 mL EtOH, and 150 mL 1N NaOH was stirred at rt for 18 h. The mixture was concentrated, acidified to pH=2 with 2N HCl, filtered and dried in vacuum to afford an off-white solid (5.96 g, 67%). LCMS-ES (Pos) m/z: 320 [M+H]⁺.

### Step 2: Preparation (4-chlorophenyl)methyl N-{4-[2-(4-hydroxy-4-methylpiperidin-1-yl)-2-oxoethyl]phenyl}carbamate

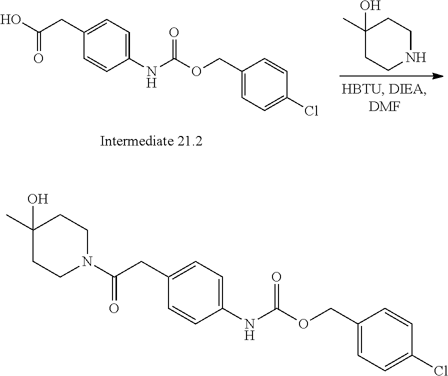

A mixture of [4-({[(4-chlorophenyl)methoxy]carbonyl}amino)phenyl]acetic acid (150 mg, 0.47 mmol, 1 equiv), 4-methylpiperidin-4-ol (54 mg, 0.47 mmol, 1 equiv), HATU (214 mg, 0.563 mmol, 1.2 equiv), DIEA (0.123 mL, 0.704 mmol, 1.5 equiv) and DMF (1 mL) was stirred at rt for 1 h. The mixture was purified by prep HPLC affording a light brown solid. LCMS-ES (Pos) m/z: 417 [M+H]⁺. ¹H NMR (400 MHz, DMSO-d₆) δ 9.73 (s, 1H), 7.46 (d, J=1.8 Hz, 4H), 7.38 (d, J=8.1 Hz, 2H), 7.12 (d, J=8.0 Hz, 2H), 5.14 (s, 2H), 4.34 (s, 1H), 3.91 (d, J=13.1 Hz, 1H), 3.57 (d, J=13.3 Hz, 1H), 3.33 (s, 2H), 3.33-3.25 (m, 1H), 3.04 (t, J=11.9 Hz, 1H), 1.41 (d, J=15.1 Hz, 1H), 1.38-1.17 (m, 2H), 1.09 (s, 2H).

Compounds in the following table were prepared in a similar manner as Compound 116, using the intermediate 21.2 and amine as listed.

| Co # | Amine | Structure, Name and Data |
|---|---|---|
| 64 | (1R,4R)-4- methoxycyclohexan-1- aminium chloride | 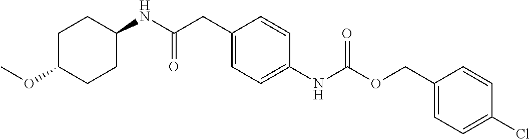 [SMILES: CO[C@H]1CC[C@H](NC(=O)Cc2ccc(NC(=O)OCc3ccc(Cl)cc3)cc2)CC1] |
| | | Compound 64: (4-chlorophenyl)methyl N-[4- |
| | | ({[(1R,4R)-4- |
| | | methoxycyclohexyl]carbamoyl}methyl)phenyl]carbamate. |
| | | LCMS-ES (Pos) m/z: 431 [M + H]⁺. ¹H NMR (400 |
| | | MHz, DMSO-d₆) δ 9.71 (s, 1H), 7.53-7.22 (m, 6H), 7.14 |
| | | (d, J = 8.1 Hz, 2H), 5.14 (d, J = 2.6 Hz, 2H), 3.33-3.50 |
| | | (m, 6H), 3.09 (s, 1H), 1.96 (s, 2H), 1.76 (s, 2H), 1.16 (q, J = |
| | | 9.9, 9.5 Hz, 4H). |
| 156 | tert-butyl piperazine- 1-carboxylate | 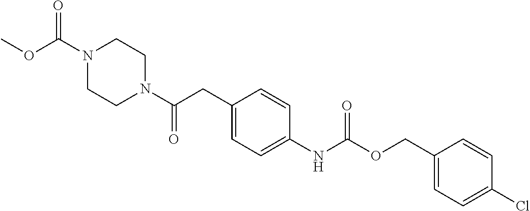 [SMILES: COC(=O)N1CCN(C(=O)Cc2ccc(NC(=O)OCc3ccc(Cl)cc3)cc2)CC1] |
| | | Compound 156: methyl 4-(2-(4-((((4- |
| | | chlorobenzyl)oxy)carbonyl)amino)phenyl)acetyl)piperazine- |
| | | 1-carboxylate. LCMS-ES (Pos) m/z: 446 [M + H]⁺. |
| | | ¹H NMR (400 MHz, DMSO-d₆) δ 9.75 (s, 1H), 7.46 (d, J = |
| | | 1.7 Hz, 4H), 7.42-7.30 (m, 2H), 7.13 (d, J = 8.3 Hz, |
| | | 2H), 5.14 (s, 2H), 3.68-3.58 (m, 5H), 3.46 (t, J = 5.8 |
| | | Hz, 3H), 3.27 (d, J = 5.0 Hz, 2H), 1.26 (q, J = 7.3, 6.6 |
| | | Hz, 3H). |
| 111 | piperdine | 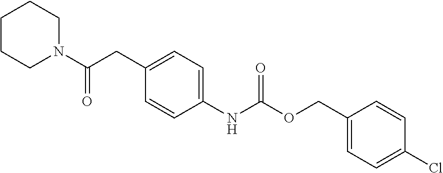 [SMILES: O=C(Nc1ccc(CC(=O)N2CCCCC2)cc1)OCc1ccc(Cl)cc1] |
| | | Compound 111: (4-chlorophenyl)methyl N-{4-[2-oxo- |
| | | 2-(piperidin-1-yl)ethyl]phenyl}carbamate Example: . |
| | | LCMS-ES (Pos) m/z: 387 (M + H). ¹H NMR (400 MHz, |
| | | DMSO-d₆) δ 9.75 (s, 1H) 7.46 (s, 4H), 7.39 (d, J = 8.2 Hz, |
| | | 2H), 7.13 (d, J = 8.2 Hz, 2H), 5.14 (s, 2H), 3.65 (s, 2H), |
| | | 3.41 (dt, J = 10.1, 5.4 Hz, 4H), 1.53 (p, J = 6.0 Hz, 2H), |
| | | 1.35 (m, 4H). |
| 119 | 4-hydroxy piperidine | 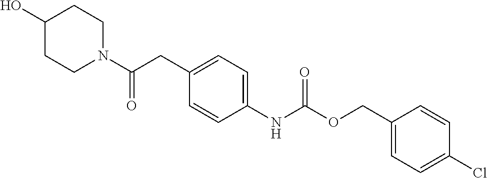 [SMILES: O=C(Nc1ccc(CC(=O)N2CCC(O)CC2)cc1)OCc1ccc(Cl)cc1] |
| | | Compound 119: (4-chlorophenyl)methyl N-{4-[2-(4- |
| | | hydroxypiperidin-1-yl)-2-oxoethyl]phenyl}carbamate. |
| | | LCMS-ES (Pos) m/z: 403 [M + H]⁺. ¹H NMR (400 MHz, |
| | | DMSO-d₆) δ 9.74 (s, 1H), 7.42 (d, J = 33.5 Hz, 5H), 7.13 |
| | | (d, J = 8.2 Hz, 2H), 5.14 (s, 2H), 4.12-3.78 (m, 2H), 3.62 |
| | | (m, 5H), 3.13 (td, J = 10.5, 10.0, 5.0 Hz, 1H), 2.97 (m, |
| | | 1H), 1.61 (dd, J = 27.8, 12.1 Hz, 2H), 1.25 (m, 2H). |
| 161 | (s)-3- methoxypiperidine | 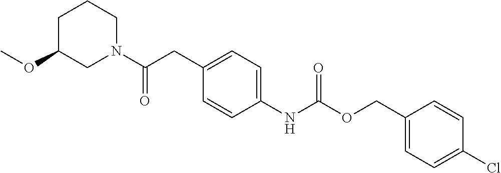 [SMILES: CO[C@H]1CCCN(C(=O)Cc2ccc(NC(=O)OCc3ccc(Cl)cc3)cc2)C1] |
| | | Compound 161: 4-chlorobenzyl (S)-(4-(2-(3- |
| | | methoxypiperidin-1-yl)-2-oxoethyl)phenyl)carbamate. |
| | | LCMS-ES (Pos) m/z: 417 [M + H]⁺. ¹H NMR (400 MHz, |
| | | DMSO-d₆) δ 9.74 (s, 1H), 7.47 (s, 4H), 7.39 (d, J = 8.0 Hz, |
| | | 2H), 7.13 (d, J = 8.0 Hz, 2H), 5.14 (s, 2H), 3.79-3.67 (m, |
| | | 3H), 3.63 (m, 2H), 3.34 (s, 3H), 3.27-3.09 (m, 2H), 1.81 |
| | | (s, 1H), 1.56 (s, 1H), 1.45 (s, 1H), 1.22 (d, J = 18.0 Hz, |
| 171 | (s)-3- hydroxypiperidine | 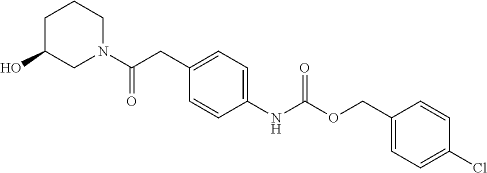 [SMILES: O=C(Nc1ccc(CC(=O)N2CCC[C@H](O)C2)cc1)OCc1ccc(Cl)cc1] |
| | | Compound 171: 4-chlorobenzyl (S)-(4-(2-(3- |
| | | hydroxypiperidin-1-yl)-2-oxoethyl)phenyl)carbamate. |
| | | LCMS-ES (Pos) m/z: 403 [M + H]⁺. ¹H NMR (400 MHz, |
| | | DMSO-d₆) δ 9.74 (s, 1H), 7.46 (d, J = 2.1 Hz, 4H), 7.38 |
| | | (d, J = 8.1 Hz, 2H), 7.16-7.08 (m, 2H), 5.14 (s, 2H), |
| | | 3.64 (q, J = 13.1, 11.2 Hz, 4H), 3.01 (d, J = 15.8 Hz, |
| | | 2H), 1.78 (s, 1H), 1.58 (d, J = 18.2 Hz, 1H), 1.38 (d, J = |
| | | 10.6 Hz, 1H), 1.22 (dt, J = 43.8, 11.7 Hz, 3H). |
| 339 | 3-amino oxetane | 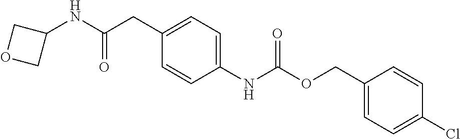 [SMILES: O=C(Cc1ccc(NC(=O)OCc2ccc(Cl)cc2)cc1)NC1COC1] |
| | | Compound 339: 4-chlorobenzyl (4-(2-(oxetan-3- |
| | | ylamino)-2-oxoethyl)phenyl)carbamate. LCMS-ES |
| | | (Pos) m/z: 375 [M + H]⁺. ¹H NMR (400 MHz, DMSO-d₆) |
| | | δ 9.73 (s, 1H), 8.77 (d, J = 6.4 Hz, 1H), 7.46 (s, 4H), 7.38 |
| | | (d, J = 8.1 Hz, 2H), 7.16 (d, J = 8.1 Hz, 2H), 5.14 (s, |
| | | 2H), 4.72 (dt, J = 21.7, 7.0 Hz, 3H), 4.40 (t, J = 6.0 Hz, |
| | | 2H), 3.33 (s, 1H). |
| 183 | 2,2-dimethylazetidine | 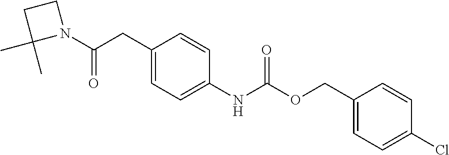 [SMILES: CC1(C)CCN1C(=O)Cc1ccc(NC(=O)OCc2ccc(Cl)cc2)cc1] |
| | | Compound 183: (4-chlorophenyl)methyl N-{4-[2-(2,2- |
| | | dimethylazetidin-1-yl)-2-oxoethyl]phenyl}carbamate. |
| | | LCMS-ES (Pos) m/z: 387 [M + H]⁺. ¹H NMR (400 MHz, |
| | | DMSO-d₆) δ 9.73 (s, 1H), 7.46 (d, J = 2.1 Hz, 4H), 7.38 |
| | | (d, J = 8.1 Hz, 2H), 7.12 (d, J = 8.6 Hz, 2H), 5.76 (d, J = |
| | | 1.8 Hz, 2H), 5.14 (s, 2H), 3.98 (m, 1H), 3.23 (s, 1H), 1.99 |
| | | (dt, J = 23.9, 7.8 Hz, 2H), 1.51 (d, J = 2.2 Hz, 1H), 1.41 |
| | | (s, 5H). |
| 194 | tert-butyl piperazine- 1-carboxylate | 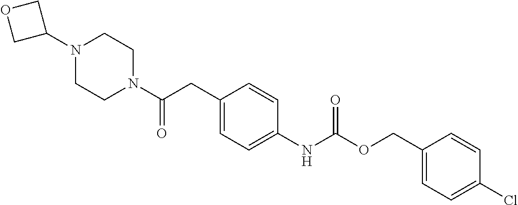 [SMILES: O=C(Nc1ccc(CC(=O)N2CCN(C3COC3)CC2)cc1)OCc1ccc(Cl)cc1] |
| | | Compound 194: (4-chlorophenyl)methyl N-(4-{2-[4- |
| | | (oxetan-3-yl)piperazin-1-yl]-2- |
| | | oxoethyl}phenyl)carbamate. LCMS-ES (Pos) m/z: 444 |
| | | [M + H]⁺. ¹H NMR (400 MHz, DMSO-d₆) δ 9.74 (s, 1H), |
| | | 7.46 (s, 4H), 7.38 (d, J = 8.1 Hz, 2H), 7.12 (d, J = 8.1 Hz, |
| | | 2H), 5.14 (s, 2H), 4.51 (s, 2H), 4.41 (s, 2H), 3.64 (s, 2H), |
| | | 3.48 (s, 3H), 2.16 (m, 5H). |
| 199 | 3-amino-6-picoline | 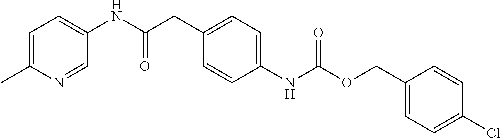 [SMILES: Cc1ccc(NC(=O)Cc2ccc(NC(=O)OCc3ccc(Cl)cc3)cc2)cn1] |
| | | Compound 199: (4-chlorophenyl)methyl N-(4-{[(6- |
| | | methylpyridin-3- |
| | | yl)carbamoyl]methyl}phenyl)carbamate. |
| | | LCMS-ES (Pos) m/z: 410 [M + H]⁺. ¹H NMR (400 MHz, |
| | | DMSO-d₆) δ 10.71 (s, 1H), 9.77 (s, 1H), 8.93 (s, 1H), 8.19 |
| | | (dd, J = 8.7, 2.4 Hz, 1H), 7.65 (d, J = 8.7 Hz, 1H), 7.46 (s, |
| | | 4H), 7.42 (d, J = 8.2 Hz, 2H), 7.24 (d, J = 8.2 Hz, 2H), |
| | | 5.76 (d, J = 1.7 Hz, 2H), 5.14 (s, 2H), 2.58 (d, J = 1.9 Hz, |
| | | 3H). |
| 202 | (S)-(+)-prolinol | 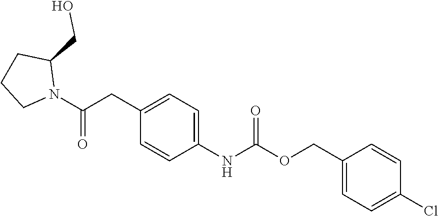 [SMILES: O=C(Nc1ccc(CC(=O)N2CCC[C@H]2CO)cc1)OCc1ccc(Cl)cc1] |
| | | Example 202: (4-chlorophenyl)methyl N-(4-{2-[(2S)-2- |
| | | (hydroxymethyl)pyrrolidin-1-yl]-2- |
| | | oxoethyl}phenyl)carbamate. LCMS-ES (Pos) m/z: 403 |
| | | (M + H). ¹H NMR (400 MHz, DMSO-d₆) δ 9.73 (s, 1H), |
| | | 7.46 (s, 4H), 7.38 (d, J = 8.2 Hz, 2H), 7.20-7.05 (m, |
| | | 2H), 5.14 (s, 2H), 3.93 (s, 1H), 3.54-3.18 (m, 8H), 1.83 |
| | | (dq, J = 31.0, 7.9 Hz, 4H). |
| 212 | 4-fluoropiperidine | 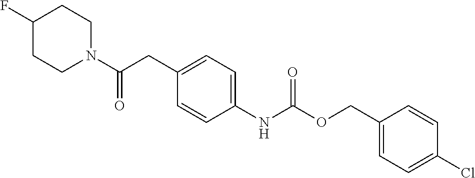 [SMILES: O=C(Nc1ccc(CC(=O)N2CCC(F)CC2)cc1)OCc1ccc(Cl)cc1] |
| | | Compound 212: (4-chlorophenyl)methyl N-{4-[2-(4- |
| | | fluoropiperidin-1-yl)-2-oxoethyl]phenyl}carbamate. |
| | | LCMS-ES (Pos) m/z: 405 [M + H]⁺. ¹H NMR (400 MHz, |
| | | DMSO-d₆) δ 9.75 (s, 1H), 7.46 (s, 4H), 7.39 (d, J = 8.1 |
| | | Hz, 2H), 7.14 (d, J = 8.2 Hz, 2H), 5.14 (s, 2H), 4.82 (d, J = |
| | | 48 Hz, 1H), 3.65 (s, 1H), 3.56 (s, 1H), 3.32 (s, 4H), |
| | | 1.73 (s, 2H), 1.59 (s, 2H), 1.53 (s, 1H). |
| 215 | 2-methylpyrolidine | 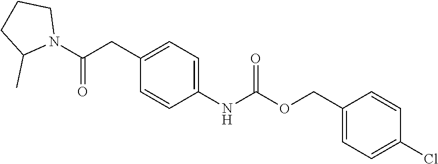 [SMILES: CC1CCCN1C(=O)Cc1ccc(NC(=O)OCc2ccc(Cl)cc2)cc1] |
| | | Compound 215: (4-chlorophenyl)methyl N-{4-[2-(2- |
| | | methylpyrrolidin-1-yl)-2-oxoethyl]phenyl}carbamate. |
| | | LCMS-ES (Pos) m/z: 387 [M + H]⁺. ¹H NMR (400 MHz, |
| | | DMSO-d₆) δ 9.73 (s, 2H), 7.46 (d, J = 2.2 Hz, 2H), 7.38 |
| | | (d, J = 8.1 Hz, 2H), 7.12 (d, J = 8.4 Hz, 2H), 5.14 (s, 2H), |
| | | 4.01 (s, 1H), 3.51 (s, 1H), 3.43 (dd, J = 18.0, 8.9 Hz, 2H), |
| | | 1.93-1.88 (m, 2H), 1.81 (s, 2H), 1.49 (s, 1H), 1.16-1.04 |
| | | (m, 4H). |
| 220 | morpholine | 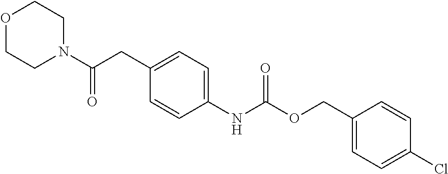 [SMILES: O=C(Nc1ccc(CC(=O)N2CCOCC2)cc1)OCc1ccc(Cl)cc1] |
| | | Compound 220: (4-chlorophenyl)methyl N-{4-[2- |
| | | (morpholin-4-yl)-2-oxoethyl]phenyl}carbamate |
| | | LCMS-ES (Pos) m/z: 389 [M + H]⁺. ¹H NMR (400 MHz, |
| | | DMSO-d₆) δ 9.74 (s, 1H), 7.46 (s, 4H), 7.39 (d, J = 8.2 Hz, |
| | | 2H), 7.13 (d, J = 8.2 Hz, 2H), 5.14 (s, 2H), 3.64 (s, 2H), |
| | | 3.52 (d, J = 4.7 Hz, 8H). |
| 221 | (1R,4R)-4- (difluoromethoxy) cyclohexan-1-amine | 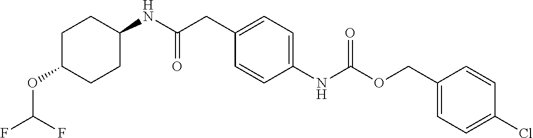 [SMILES: O=C(Cc1ccc(NC(=O)OCc2ccc(Cl)cc2)cc1)N[C@H]1CC[C@H](OC(F)F)CC1] |
| | | Compound 221: (4-chlorophenyl)methyl N-[4- |
| | | ({[(1r,4r)-4- |
| | | (difluoromethoxy)cyclohexyl]carbamoyl}methyl)phenyl] |
| | | carbamate. LCMS-ES (Pos) m/z:467 (M + H). ¹H NMR |
| | | (400 MHz, DMSO-d₆) δ 9.74 (s, 2H), 7.72-6.97 (m, 7H), |
| | | 5.14 (s, 2H), 4.20 (t, J = 15.2 Hz, 1H), 3.78 (d, J = 13.6 |
| | | Hz, 1H), 3.62 (s, 2H), 2.63 (d, J = 11.9 Hz, 1H), 2.27 (t, J = |
| | | 11.8 Hz, 1H), 1.82-0.94 (m, 5H), 0.80 (dd, J = 19.9, |
| | | 6.6 Hz, 3H). |
| 229 | (R)-3- methoxypiperidine | 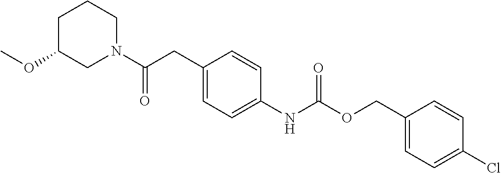 [SMILES: CO[C@@H]1CCCN(C(=O)Cc2ccc(NC(=O)OCc3ccc(Cl)cc3)cc2)C1] |
| | | Compound 229: (4-chlorophenyl)methyl N-(4-{2- |
| | | [(3R)-3-methoxypiperidin-1-yl]-2- |
| | | oxoethyl}phenyl)carbamate. LCMS-ES (Pos) m/z: 417 |
| | | [M + H]⁺. ¹H NMR (400 MHz, DMSO-d₆) δ 9.74 (s, 1H), |
| | | 7.46 (s, 4H), 7.39 (d, J = 8.1 Hz, 2H), 7.17-7.09 (m, 2H), |
| | | 5.14 (s, 2H), 3.71-3.50 (m, 2H), 3.39 (m, 3H), 3.24 (s, |
| | | 3H), 3.16 (d, J = 13.0 Hz, 2H), 1.81 (s, 1H), 1.56 (s, 1H), |
| | | 1.45 (s, 1H), 1.22 (d, J = 16.3 Hz, 1H). |
| 235 | 2-oxopiperazine | 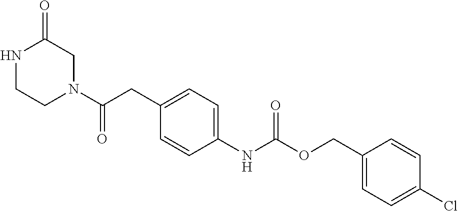 [SMILES: O=C1CN(C(=O)Cc2ccc(NC(=O)OCc3ccc(Cl)cc3)cc2)CCN1] |
| | | Compound 235: (4-chlorophenyl)methyl N-{4-[2-oxo- |
| | | 2-(3-oxopiperazin-1-yl)ethyl]phenyl}carbamate. |
| | | LCMS-ES (Pos) m/z: 402 [M + H]⁺. ¹H NMR (400 MHz, |
| | | DMSO-d₆) δ 9.75 (s, 1H), 7.46 (s, 4H), 7.39 (d, J = 8.1 |
| | | Hz, 2H), 7.13 (t, J = 9.3 Hz, 2H), 5.14 (s, 2H), 4.04 (s, |
| | | 1H), 3.94 (s, 1H), 3.68-3.56 (m, 3H), 3.32 (s, 2H), 3.13 |
| | | (d, J = 14.6 Hz, 2H). |
| 237 | (3R)-3-piperidinol | 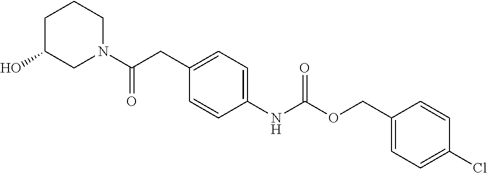 [SMILES: O=C(Nc1ccc(CC(=O)N2CCC[C@@H](O)C2)cc1)OCc1ccc(Cl)cc1] |
| | | Compound 237: (4-chlorophenyl)methyl N-(4-{2- |
| | | [(3R)-3-hydroxypiperidin-1-yl]-2- |
| | | oxoethyl}phenyl)carbamate |
| | | LCMS-ES (Pos) m/z: 403 (M + H) ¹H NMR (400 MHz, |
| | | DMSO-d₆) δ 9.74 (s, 1H), 7.46 (d, J = 2.3 Hz, 4H), 7.38 |
| | | (d, J = 8.1 Hz, 2H), 7.11 (dd, J = 8.3, 3.8 Hz, 2H), 5.14 (s, |
| | | 2H), 4.83 (s, 1H), 4.13 (d, J = 12.0 Hz, 1H), 3.64 (q, J = |
| | | 13.3, 11.2 Hz, 3H), 2.99 (s, 1H), 2.55 (m, 1H), 1.78 (s, |
| | | 1H), 1.58 (d, J = 18.0 Hz, 1H), 1.38 (d, J = 10.6 Hz, 1H), |
| | | 1.27 (t, J = 11.4 Hz, 1H), 1.15 (d, J = 12.4 Hz, 1H). |
| 240 | 4-methyl piperidine | 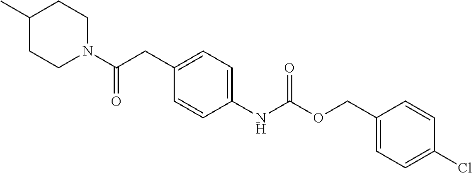 [SMILES: CC1CCN(C(=O)Cc2ccc(NC(=O)OCc3ccc(Cl)cc3)cc2)CC1] |
| | | Compound 240: (4-chlorophenyl)methyl N-{4-[2-(4- |
| | | methylpiperidin-1-yl)-2-oxoethyl]phenyl}carbamate. |
| | | LCMS-ES (Pos) m/z: 401 [M + H]⁺. ¹H NMR (400 MHz, |
| | | DMSO-d₆) δ 9.73 (s, 1H), 7.55-6.97 (m, 86H), 5.14 (s, |
| | | 2H), 4.34 (d, J = 13.0 Hz, 1H), 3.88 (d, J = 13.5 Hz, 1H), |
| | | 3.34 (s, 3H), 2.92 (t, J = 13.1 Hz, 1H), 1.69-1.46 (m, 3H), |
| | | 0.86 (m,5H). |
| 244 | (3S)-3- methylpyrrolidine | 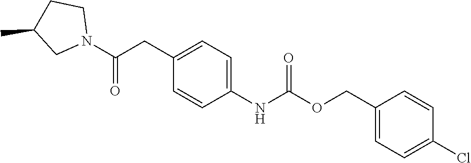 [SMILES: C[C@H]1CCN(C(=O)Cc2ccc(NC(=O)OCc3ccc(Cl)cc3)cc2)C1] |
| | | Compound 244: (4-chlorophenyl)methyl N-(4-{2- |
| | | [(3S)-3-methylpyrrolidin-1-yl]-2- |
| | | oxoethyl}phenyl)carbamate |
| | | LCMS-ES (Pos) m/z: 387 [M + H]⁺. ¹H NMR (400 MHz, |
| | | DMSO-d₆) δ 9.73 (s, 1H), 7.46 (d, J = 2.2 Hz, 4H), 7.38 |
| | | (d, J = 8.1 Hz, 2H), 7.13 (d, J = 8.1 Hz, 2H), 5.14 (s, 2H), |
| | | 3.69-3.54 (m, 1H), 3.51-3.37 (m, 1H), 3.35 (s, 2H), |
| | | 3.20 (q, J = 9.8, 9.3 Hz, 1H), 2.96 (t, J = 9.2 Hz, 1H), 2.25- |
| | | 2.13 (m, 1H), 1.99-1.91 (m, 1H), 1.57-1.39 (m, 1H),, |
| | | 0.99 (m, 3H). |
| 256 | (3R)-3- methylpiperidine | 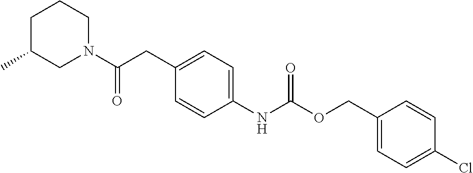 [SMILES: C[C@@H]1CCCN(C(=O)Cc2ccc(NC(=O)OCc3ccc(Cl)cc3)cc2)C1] |
| | | Compound 256: (4-chlorophenyl)methyl N-(4-{2- |
| | | [(3R)-3-methylpiperidin-1-yl]-2- |
| | | oxoethyl}phenyl)carbamate. LCMS-ES (Pos) m/z: 401 |
| | | (M + H). ¹H NMR (400 MHz, DMSO-d₆) δ 9.74 (s, 1H), |
| | | 7.46 (s, 4H), 7.38 (d, J = 8.2 Hz, 2H), 7.13 (d, J = 8.1 Hz, |
| | | 2H), 5.14 (s, 2H), 4.20 (t, J = 15.2 Hz, 1H), 3.78 (d, J = |
| | | 13.6 Hz, 1H), 3.62 (s, 2H), 2.61 (t, J = 12.4 Hz, 1H), 2.27 |
| | | (t, J = 11.8 Hz, 1H), 1.68 (d, J = 12.3 Hz, 1H), 1.54 (dd, J = |
| | | 27.6, 12.6 Hz, 1H), 1.24 (s, 1H), 1.06 (d, J = 12.4 Hz, |
| | | 1H), 0.80 (m, 4H). |
| 257 | (2S)-2- piperidinemethanol | 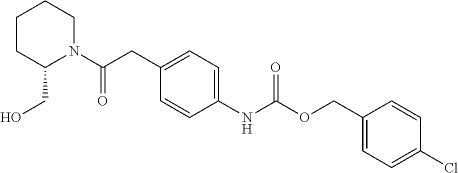 [SMILES: O=C(Nc1ccc(CC(=O)N2CCCC[C@H]2CO)cc1)OCc1ccc(Cl)cc1] |
| | | Compound 257: (4-chlorophenyl)methyl N-(4-{2- |
| | | [(2S)-2-(hydroxymethyl)piperidin-1-yl]-2- |
| | | oxoethyl}phenyl)carbamate. LCMS-ES (Pos) m/z: 417 |
| | | [M + H]⁺. ¹H NMR (400 MHz, DMSO-d₆) δ 9.73 (s, 1H), |
| | | 7.46 (d, J = 2.3 Hz, 4H), 7.39 (d, J = 8.2 Hz, 2H), 7.12 (s, |
| | | 2H), 5.14 (s, 1H), 4.83 (s, 1H), 4.64 (m, 1H), 4.53 (m, |
| | | 1H), 4.35 (d, J = 13.8 Hz, 1H), 3.97 (s, 1H), 3.75 (t, J = |
| | | 17.3 Hz, 2H), 3.46 (s, 1H), 3.32 (d, J = 2.2 Hz, 3H), 1.59 |
| | | (s, 1H), 1.46 (s, 1H), 1.22 (s, 2H). |
| 261 | (S)-3-methylpiperidine | 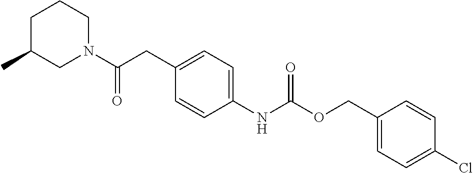 [SMILES: C[C@H]1CCCN(C(=O)Cc2ccc(NC(=O)OCc3ccc(Cl)cc3)cc2)C1] |
| | | Compound 261. (4-chlorophenyl)methyl N-(4-{2-[(3S)- |
| | | 3-methylpiperidin-1-yl]-2-oxoethyl}phenyl)carbamate |
| | | LCMS-ES (Pos) m/z: 401 (M + H). ¹H NMR (400 MHz, |
| | | DMSO-d₆) δ 9.74 (s, 1H), 7.47 (s, 1H), 7.46 (s, 3H), 7.39 |
| | | (d, J = 8.0 Hz, 2H), 7.13 (d, J = 8.0 Hz, 2H), 5.14 (s, 2H), |
| | | 3.79-3.67 (m, 1H), 3.63 (s, 1H), 3.34 (s, 2H), 3.27-3.09 |
| | | (m, 3H), 1.81 (s, 1H), 1.56 (s, 1H), 1.45 (s, 1H), 1.22 (d, J = |
| | | 18.0 Hz, 1H), 0.8 (m, 3H). |
| 274 | 4,4-difluoropiperidine | 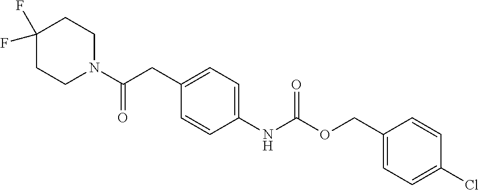 [SMILES: O=C(Nc1ccc(CC(=O)N2CCC(F)(F)CC2)cc1)OCc1ccc(Cl)cc1] |
| | | Compound 274: (4-chlorophenyl)methyl N-{4-[2-(4,4- |
| | | difluoropiperidin-1-yl)-2-oxoethyl]phenyl}carbamate. |
| | | LCMS-ES (Pos) m/z: 423 [M + H]⁺. ¹H NMR (400 MHz, |
| | | DMSO-d₆) δ 9.75 (s, 1H), 7.46 (s, 4H), 7.40 (d, J = 8.0 |
| | | Hz,2H), 7.14 (d, J = 8.1 Hz, 2H), 5.14 (s, 2H), 3.70 (s, 2H), |
| | | 3.56 (d, J = 6.1 Hz, 4H), 1.91-1.81 (m, 4H). |
| 280 | (1S,4S)-4- methoxycyclo- hexanamine | 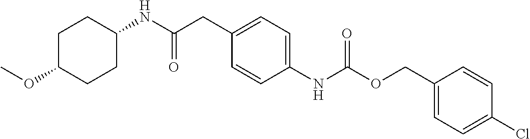 [SMILES: CO[C@H]1CC[C@@H](NC(=O)Cc2ccc(NC(=O)OCc3ccc(Cl)cc3)cc2)CC1] |
| | | Compound 280: (4-chlorophenyl)methyl N-[4- |
| | | ({[(1s,4s)-4- |
| | | methoxycyclohexyl]carbamoyl}methyl)phenyl]carbamate. |
| | | LCMS-ES (Pos) m/z: 431 [M + H]⁺. ¹H NMR (400 |
| | | MHz, DMSO-d₆) δ 9.71 (s, 1H), 7.88 (d, J = 7.7 Hz, 2H), |
| | | 7.46 (d, J = 2.3 Hz, 4H), 7.36 (d, J = 8.1 Hz, 2H), 7.15 (d, |
| | | J = 8.1 Hz, 2H), 5.14 (s, 2H), 3.57 (s, 1H), 3.33 (s, 3H), |
| | | 3.21 (d, J = 2.5 Hz, 2H), 1.74 (s, 2H), 1.46 (d, J = 6.0 Hz, |
| | | 6H). |
| 282 | tert-butyl piperazine- 1-carboxylate | 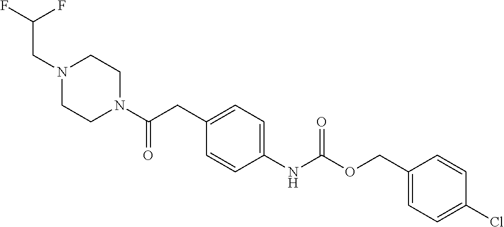 [SMILES: O=C(Nc1ccc(CC(=O)N2CCN(CC(F)F)CC2)cc1)OCc1ccc(Cl)cc1] |
| | | Compound 282: (4-chlorophenyl)methyl N-(4-{2-[4- |
| | | (2,2-difluoroethyl)piperazin-1-yl]-2- |
| | | oxoethyl}phenyl)carbamate. LCMS-ES (Pos) m/z: 452 |
| | | [M + H]⁺. ¹H NMR (400 MHz, DMSO-d₆) δ 9.74 (s, 1H), |
| | | 7.46 (d, J = 2.5 Hz, 4H), 7.39 (d, J = 8.0 Hz, 2H), 7.12 |
| | | (d, J = 8.2 Hz, 2H), 6.17 (m, 1H), 5.14 (s, 2H), 3.44 (m, |
| | | 8H), 3.63 (s, 2H), 2.80 (d, J = 18.4 Hz, 1H), 2.72 (s, 1H). |
| 283 | 4-methoxypiperidine | 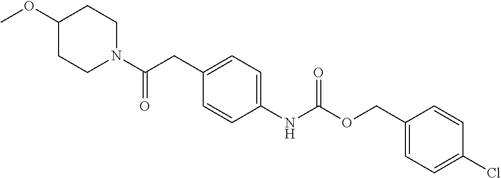 [SMILES: COC1CCN(C(=O)Cc2ccc(NC(=O)OCc3ccc(Cl)cc3)cc2)CC1] |
| | | Compound 283: (4-chlorophenyl)methyl N-{4-[2-(4- |
| | | methoxypiperidin-1-yl)-2-oxoethyl]phenyl}carbamate. |
| | | LCMS-ES (Pos) m/z: 417 [M + H]⁺. ¹H NMR (400 MHz, |
| | | DMSO-d₆) δ 9.74 (s, 1H), 7.46 (s, 4H), 7.39 (d, J = 8.2 Hz, |
| | | 2H), 7.13 (d, J = 8.2 Hz, 2H), 5.14 (s, 2H), 3.90-3.73 (m, |
| | | 1H), 3.67 (s, 3H), 3.32 (s, 2H), 3.20-3.12 (m, 1H), 3.06 |
| | | (td, J = 10.0, 9.3, 5.1 Hz, 1H), 2.70 (d, J = 1.3 Hz, 2H), |
| | | 1.84-1.56 (m, 2H), 1.38-1.02 (m, 2H). |
| 287 | (2R)-2- piperidinemethanoL | 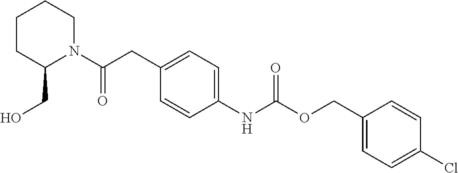 [SMILES: O=C(Nc1ccc(CC(=O)N2CCCC[C@@H]2CO)cc1)OCc1ccc(Cl)cc1] |
| | | Compound 287: (4-chlorophenyl)methyl N-(4-{2- |
| | | [(2R)-2-(hydroxymethyl)piperidin-1-yl]-2- |
| | | oxoethyl}phenyl)carbamate. |
| | | LCMS-ES (Pos) m/z: 417 [M + H]⁺. ¹H NMR (400 MHz, |
| | | DMSO-d₆) δ 9.74 (s, 1H), 7.46 (d, J = 2.3 Hz, 4H), 7.39 |
| | | (d, J = 8.1 Hz, 2H), 7.11 (d, J = 8.0 Hz, 2H), 5.14 (s, 2H), |
| | | 4.83 (s, 1H), 4.53 (s, 1H), 4.35 (d, J = 13.9 Hz, 1H), 3.97 |
| | | (s, 1H), 3.73 (dd, J = 33.2, 14.4 Hz, 1H), 3.62 (s, 2H), 3.55 |
| | | (d, J = 16.3 Hz, 1H), 3.46 (s, 1H), 1.59 (s, 1H), 1.46 (s, |
| | | 2H) 1.24 (m, 2H). |
| 300 | (pyrazin-2- ylmethyl)amine | 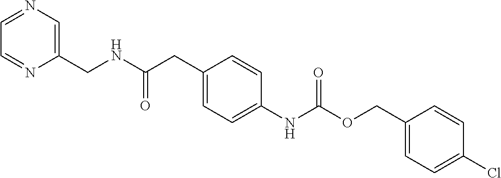 [SMILES: O=C(Cc1ccc(NC(=O)OCc2ccc(Cl)cc2)cc1)NCc1cnccn1] |
| | | Compound 300: (4-chlorophenyl)methyl N-(4- |
| | | {[(pyrazin-2- |
| | | ylmethyl)carbamoyl]methyl}phenyl)carbamate |
| | | LCMS-ES (Pos) m/z: 411 [M + H]⁺. ¹H NMR (400 MHz, |
| | | DMSO-d₆) δ 9.74 (s, 1H), 8.66 (s, 1H), 8.58 (s, 1H), 8.51 |
| | | (d, J = 10.0 Hz, 2H), 7.46 (s, 4H), 7.39 (d, J = 7.9 Hz, |
| | | 2H), 7.19 (d, J = 8.1 Hz, 2H), 5.14 (s, 2H), 4.41 (d, J = |
| | | 5.8 Hz, 2H), 3.44 (s, 2H). |
| 308 | 4- aminotetrahydropyran | 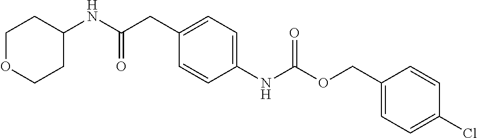 [SMILES: O=C(Cc1ccc(NC(=O)OCc2ccc(Cl)cc2)cc1)NC1CCOCC1] |
| | | Compound 308: (4-chlorophenyl)methyl N-(4-{[(oxan- |
| | | 4-yl)carbamoyl]methyl}phenyl)carbamate LCMS-ES |
| | | (Pos) m/z: 403 (M + H). ¹H NMR (400 MHz, Chloroform- |
| | | d) δ 7.29 (d, J = 6.8 Hz, 4H), 7.19 (s, 3H), 7.12 (d, J = |
| | | 8.1 Hz, 2H), 6.61 (s, 1H), 5.10 (s, 2H), 3.81 (d, J = 12.2 |
| | | Hz, 1H), 3.45 (s, 2H), 3.36 (t, J = 11.6 Hz, 2H), 1.53 (s, |
| | | 4H), 1.34-1.16 (m, 2H). |
| 317 | (R)-(+)-3- hydroxypyrrolidine | 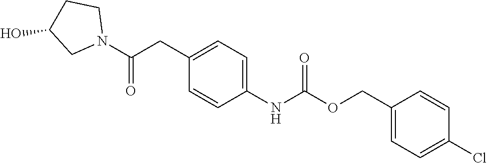 [SMILES: O=C(Nc1ccc(CC(=O)N2CC[C@@H](O)C2)cc1)OCc1ccc(Cl)cc1] |
| | | Compound 317: (4-chlorophenyl)methyl N-(4-{2- |
| | | [(3R)-3-hydroxypyrrolidin-1-yl]-2- |
| | | oxoethyl}phenyl)carbamate LCMS-ES (Pos) m/z: 389 |
| | | [M + H]⁺. ¹H NMR (400 MHz, DMSO-d₆) δ 9.73 (s, 1H), |
| | | 7.46 (s, 4H), 7.38 (d, J = 8.1 Hz, 2H), 7.13 (d, J = 8.1 Hz, |
| | | 2H), 5.14 (s, 2H), 4.99 (s, 1H), 4.23 (s, 1H), 3.33 (m, |
| | | 5H), 3.31-3.21 (m, 1H), 1.82 (m, 1H), 1.73 (s, 1H). |
| 319 | (S)-(−)-3- hydroxypyrrolidine | 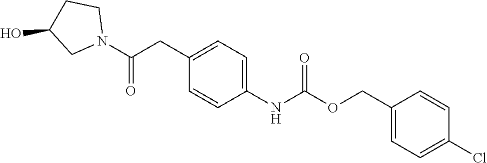 [SMILES: O=C(Nc1ccc(CC(=O)N2CC[C@H](O)C2)cc1)OCc1ccc(Cl)cc1] |
| | | Compound 319: (4- chlorophenyl)methyl N-(4-{2- |
| | | [(3S)-3-hydroxypyrrolidin-1-yl]-2- |
| | | oxoethyl}phenyl)carbamate. LCMS-ES (Pos) m/z: 389 |
| | | [M + H]⁺. ¹H NMR (400 MHz, DMSO-d₆) δ 9.73 (s, 1H), |
| | | 7.46 (s, 4H), 7.38 (d, J = 8.1 Hz, 2H), 7.13 (d, J = 8.1 Hz, |
| | | 2H), 5.14 (s, 2H), 4.99 (s, 1H), 4.89 (s, 1H), 4.29 (s, 1H), |
| | | 4.23 (s, 1H), 3.33 (s, 5H), 3.31-3.21 (m, 1H), 1.73 (s, |
| | | 1H). ¹H NMR (400 MHz, DMSO-d₆) δ 9.73 (s, 1H), 7.46 |
| | | (s, 4H), 7.38 (d, J = 8.1 Hz, 2H), 7.13 (d, J = 8.1 Hz, |
| | | 2H), 5.14 (s, 2H), 4.99 (s, 1H), 4.23 (s, 1H), 3.33 (m, |
| | | 5H), 3.31-3.21 (m, 1H), 1.82 (m, 1H), 1.73 (s, 1H). |
| 323 | (6-methyl-3- pyridinyl)methylamine | 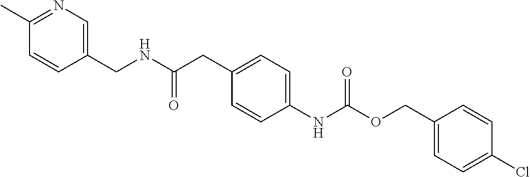 [SMILES: Cc1ccc(CNC(=O)Cc2ccc(NC(=O)OCc3ccc(Cl)cc3)cc2)cn1] |
| | | Compound 323: (4-chlorophenyl)methyl N-[4-({[(6- |
| | | methylpyridin-3- |
| | | yl)methyl]carbamoyl}methyl)phenyl]carbamate. |
| | | LCMS-ES (Pos) m/z: 424 [M + H]⁺. ¹H NMR (400 MHz, |
| | | Methanol-d₄) δ 9.33 (s, 1H), 8.61 (d, J = 6.4 Hz, 1H), 8.46 |
| | | (s, 1H), 8.28 (dd, J = 8.3, 1.9 Hz, 1H), 7.81 (d, J = 8.3 Hz, |
| | | 1H), 7.37 (t, J = 7.5 Hz, 6H), 7.20 (d, J = 8.1 Hz, 2H), 5.15 |
| | | (s, 2H), 4.48 (d, J = 4.6 Hz, 2H), 3.52 (s, 2H), 2.72 (s, 3H). |
| 332 | 1-methyl-2- piperazinone | 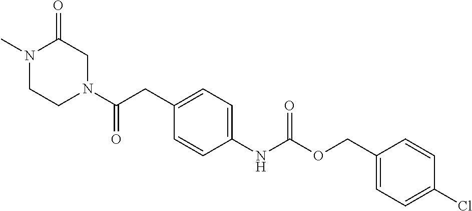 [SMILES: CN1CCN(C(=O)Cc2ccc(NC(=O)OCc3ccc(Cl)cc3)cc2)CC1=O] |
| | | Compound 332: (4-chlorophenyl)methyl N-{4-[2-(4- |
| | | methyl-3-oxopiperazin-1-yl)-2- |
| | | oxoethyl]phenyl}carbamate |
| | | LCMS-ES (Pos) m/z: 416 [M + H]⁺. ¹H NMR (400 MHz, |
| | | DMSO-d₆) δ 9.75 (s, 1H), 7.46 (s, 4H), 7.39 (d, J = 8.1 Hz, |
| | | 2H), 7.13 (dd, J = 13.4, 8.2 Hz, 2H), 5.76 (d, J = 1.3 Hz, |
| | | 1H), 5.14 (s, 2H), 3.99 (s, 1H), 3.78-3.56 (m, 3H), 3.28 |
| | | (d, J = 5.8 Hz, 1H), 3.21-3.09 (m, 1H), 2.84 (d, J = 4.9 |
| | | Hz, 1H), 1.26 (q, J = 7.3, 6.6 Hz, 3H). |
| 338 | (1R,4R)-4- (trifluoromethoxy) cyclohexan-1-amine | 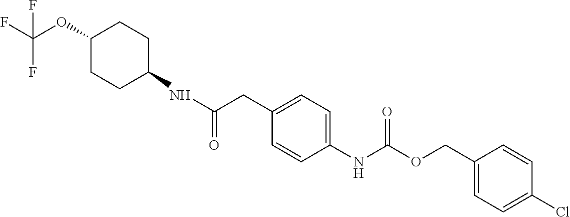 [SMILES: O=C(Cc1ccc(NC(=O)OCc2ccc(Cl)cc2)cc1)N[C@H]1CC[C@H](OC(F)(F)F)CC1] |
| | | Compound 338: (4-chlorophenyl)methyl N-[4- |
| | | ({[(1r,4r)-4- |
| | | (trifluoromethoxy)cyclohexyl]carbamoyl}methyl)phenyl] |
| | | carbamate LCMS-ES (Pos) m/z: 485 [M + H]⁺. ¹H |
| | | NMR (400 MHz, DMSO-d₆) δ 9.72 (s, 1H), 7.94 (d, J = |
| | | 7.5 Hz, 1H), 7.46 (d, J = 2.6 Hz, 4H), 7.36 (d, J = 8.1 Hz, |
| | | 2H), 7.14 (d, J = 8.0 Hz, 2H), 5.14 (s, 2H), 4.37 (s, 1H), |
| | | 3.36 (s, 2H), 3.30 (s, 1H), 2.03-1.94 (m, 2H), 1.85-1.76 |
| | | (m, 2H), 1.55 (q, J = 12.0 Hz, 2H), 1.31 (q, J = 12.3 Hz, |
| | | 2H). |
| 264 | N,6-dimethylpyridin- 3-amine | 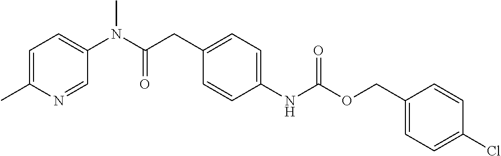 [SMILES: Cc1ccc(N(C)C(=O)Cc2ccc(NC(=O)OCc3ccc(Cl)cc3)cc2)cn1] |
| | | Compound 264: (4-chlorophenyl)methyl N-(4- |
| | | {[methyl(6-methylpyridin-3- |
| | | yl)carbamoyl]methyl}phenyl)carbamate. LCMS-APCI |
| | | (POS.) m/z: 424.1 [M + H]⁺. ¹H NMR (400 MHz, 60° C., |
| | | Methanol-d₆) δ 8.25 (s, 1H), 7.58 (d, J = 8.1 Hz, 1H), 7.43- |
| | | 7.27 (m, 7H), 6.94 (s, 2H), 5.15 (s, 2H), 3.52 (bs, 2H), |
| | | 3.27 (s, 3H), 2.56 (d, J = 1.9 Hz, 3H). |
| 178 | 2-methyl-2,5- diazabicyclo[4.1.0] heptane | 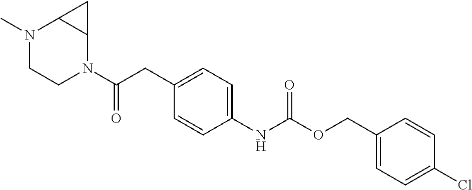 [SMILES: CN1CCN(C(=O)Cc2ccc(NC(=O)OCc3ccc(Cl)cc3)cc2)C2CC21] |
| | | Compound 178: 4-chlorobenzyl (4-(2-(5-methyl-2,5- |
| | | diazabicyclo[4.1.0]heptan-2-yl)-2- |
| | | oxoethyl)phenyl)carbamate. LCMS-APCI (POS.) m/z: |
| | | 414 [M + H]⁺. ¹H NMR (400 MHz, DMSO-d₆) δ 9.73 (s, |
| | | 1H), 7.46 (m, 4H), 7.39 (d, J = 8.1 Hz, 2H), 7.13 (dd, J = |
| | | 18.3, 8.1 Hz, 2H), 5.14 (s, 2H), 4.05-3.60 (m, 2H), 3.32 (s, |
| | | 3H), 2.96 (d, J = 9.9 Hz, 1H), 2.61 (s, 2H), 2.35-2.26 (m, |
| | | 2H), 2.03 (t, J = 10.9 Hz, 1H), 1.24 (s, 1H), 0.63 (m, 1H). |
| 273 | 5-fluoropyridin-3- amine | 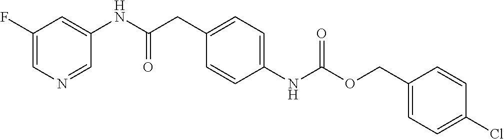 [SMILES: O=C(Cc1ccc(NC(=O)OCc2ccc(Cl)cc2)cc1)Nc1cncc(F)c1] |
| | | Compound 273: (4-chlorophenyl)methyl N-(4-{[(5- |
| | | fluoropyridin-3- |
| | | yl)carbamoyl]methyl}phenyl)carbamate. LCMS-APCI |
| | | (POS.) m/z: 414.0 [M + H]⁺. ¹H NMR (400 MHz, DMSO- |
| | | d₆) δ 10.60 (s, 1H), 9.76 (s, 1H), 8.53 (s, 1H), 8.26 (s, 1H), |
| | | 8.05 (d, J = 11.4 Hz, 1H), 7.49-7.44 (m, 4H), 7.41 (d, J = |
| | | 8.1 Hz, 2H), 7.23 (d, J = 8.2 Hz, 2H), 5.13 (s, 2H), 3.62 (s, |
| | | 2H). |
| 312 | 2-piperazinone | 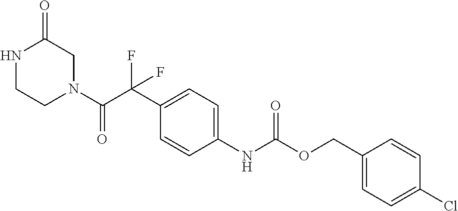 [SMILES: O=C1CN(C(=O)C(F)(F)c2ccc(NC(=O)OCc3ccc(Cl)cc3)cc2)CCN1] |
| | | Compound 312: (4-chlorophenyl)methyl N-{4-[1,1- |
| | | difluoro-2-oxo-2-(3-oxopiperazin-1- |
| | | yl)ethyl]phenyl}carbamate. LCMS-ES (Pos) m/z: 438 |
| | | [M + H]⁺. ¹H NMR (400 MHz, Methanol-d₄) δ 7.62 (d, J = |
| | | 8.4 Hz, 2H), 7.49 (d, J = 7.9 Hz, 2H), 7.39 (q, J = 8.2 Hz, |
| | | 4H), 5.17 (s, 2H), 4.23 (s, 1H), 4.06 (s, 1H), 3.87 (s, 1H), |
| | | 3.66 (s, 1H), 3.09 (s, 2H). |

### Example P

### Preparation of (4-chlorophenyl)methyl N-{4-[(3-methyl-1,2,4-oxadiazol-5-yl)methyl]phenyl}carbamate (Compound 344)

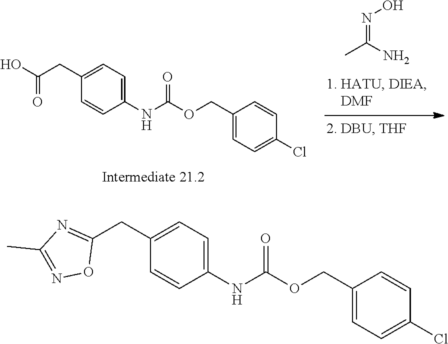

To a solution of [4-({[(4-chlorophenyl)methoxy]carbonyl}amino)phenyl]acetic acid (148 mg, 0.46 mmol) and N,N-diisopropylethylamine (0.32 mL, 1.85 mmol) in DMF (1.5 mL) was added (Z)—N′-hydroxyethanimidamide (44.6 mg, 0.60 mmol) and 1-[bis(dimethylamino)methylene]-1H-1,2,3-triazolo[4,5-b]pyridinium 3-oxid hexafluorophosphate (228.8 mg, 0.60 mmol). The mixture was stirred at rt for 1.5 h, diluted with water, saturated sodium bicarbonate, and extracted with DCM. The combined organic layers were dried over sodium sulfate, concentrated, and used without further purification. To a crude solution of (Z)-(1-aminoethylidene)amino 2-[4-({[(4-chlorophenyl)methoxy]carbonyl}amino)phenyl]acetate (173.9 mg, 0.46 mmol) in THF (3 mL) was added DBU (0.21 mL, 1.39 mmol). The mixture was stirred at 50° C. for 16 h, cooled, filtered, and purified by reverse phase prep HPLC using a gradient of 3-40% water/acetonitrile with 0.1% formic acid to yield product (4-chlorophenyl)methyl N-{4-[(3-methyl-1,2,4-oxadiazol-5-yl)methyl]phenyl}carbamate (13 mg, 0.04 mmol, 8% yield) over 2 steps. LCMS-APCI (POS.) m/z: 358.4 [M+H]⁺. ¹H NMR (400 MHz, DMSO-d₆) δ 9.80 (s, 1H), 7.49-7.44 (m, 4H), 7.43 (d, J=7.6 Hz, 2H), 7.23 (d, J=8.0 Hz, 2H), 5.13 (s, 2H), 4.21 (s, 2H), 2.29 (d, J=2.4 Hz, 3H).

### LHS Sulfonamide Synthesis

### Example Q

### Preparation of 4-chlorobenzyl (4-(N-methyl-N-(oxetan-3-yl)sulfamoyl)phenyl)carbamate (Compound 352)

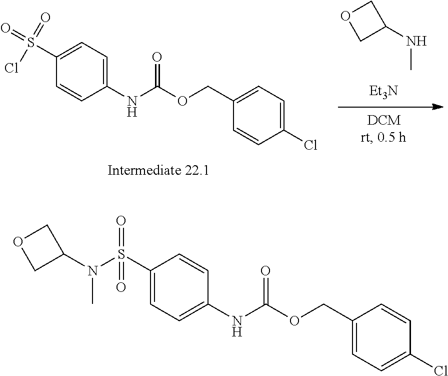

A mixture of (4-chlorophenyl)methyl N-[4-(chlorosulfonyl)phenyl]carbamate (50 mg, 0.14 mmol, 1.0 eqiv), triethylamine (0.028 g, 0.27 mmol, 2.0 eq), and amine (1.5 eqiv) was stirred in DCM (1 mL) for 30 min. The reaction mixture was concentrated, resuspended in MeOH, and purified on reverse phase HPLC eluted with 0-95% acetonitrile:water w/0.1% formic acid) to give the desired product LCMS-APCI (POS.) m/z: 411.1 [M+H]⁺. 1H NMR (400 MHz, DMSO-d6) δ 10.33 (s, 1H), 7.68 (s, 4H), 7.47 (d, J=2.1 Hz, 4H), 5.18 (s, 2H), 4.54 (s, 4H), 3.17 (d, J=5.2 Hz, 1H), 2.66 (s, 3H).

Compounds in the following table were prepared in a similar manner as Compound 352, using the intermediate 22.1 and amine as listed.

| Co | | |
|---|---|---|
| # | amine | Structure, Name and Data |
| 335 | N-methyl-1- phenylmethanamine | 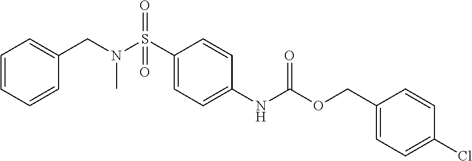 [SMILES: CN(Cc1ccccc1)S(=O)(=O)c1ccc(NC(=O)OCc2ccc(Cl)cc2)cc1] Compound 335: 4-chlorobenzyl (4-(N-benzyl-N- methylsulfamoyl)phenyl)carbamate. LCMS-APCI (POS.) m/z: 444.0 [M + H]⁺. 1H NMR (400 MHZ, DMSO-d6) δ 10.32 (s, 1H), 7.80-7.69 (m, 4H), 7.48 (s, 4H), 7.36 (d, J = 7.0 Hz, 2H), 7.30 (d, J = 7.7 Hz, 3H), 5.19 (s, 2H), 4.08 (s, 2H). |
| 337 | (R)-N- methyltetrahydrofuran- 3-amine | 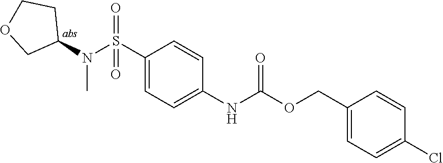 [SMILES: CN([C@@H]1CCOC1)S(=O)(=O)c1ccc(NC(=O)OCc2ccc(Cl)cc2)cc1] Compound 337: 4-chlorobenzyl (R)-(4-(N-methyl-N- (tetrahydrofuran-3-yl)sulfamoyl)phenyl)carbamate. LCMS-APCI (POS.) m/z: 427.0 [M + H]⁺. 1H NMR (400 MHZ, DMSO-d6) δ 10.31 (s, 1H), 7.70 (q, J = 8.5 Hz, 4H), 7.47 (d, J = 1.9 Hz, 4H), 5.18 (s, 2H), 4.50 (s, 2H), 3.75 (s, 2H), 2.61 (d, J = 1.7 Hz, 3H), 1.88 (s, 2H), 1.49 (s, 1H). |
| 342 | N-methyltetrahydrofuran- 3-amine | 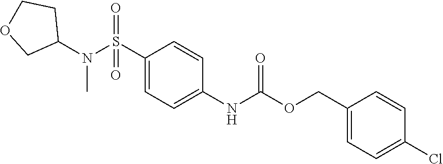 [SMILES: CN(C1CCOC1)S(=O)(=O)c1ccc(NC(=O)OCc2ccc(Cl)cc2)cc1] Compound 342: 4-chlorobenzyl (4-(N- methyl-N-(tetrahydrofuran-3- yl)sulfamoyl)phenyl)carbamate. LCMS-APCI (POS.) m/z: 424.1 [M + H]⁺. 1H NMR (400 MHZ, DMSO-d6) δ 10.31 (s, 1H), 7.70 (q, J = 8.7 Hz, 4H), 7.48 (d, J = 2.0 Hz, 4H), 5.19 (s, 2H), 4.50 (s, 2H), 3.76 (s, 2H), 2.62 (s, 3H), 1.87 (s, 1H), 1.50 (s, 1H). |

### Example R

### Preparation of oxazol-5-ylmethyl (4-((1-(dimethylcarbamoyl)piperidin-4-yl)methyl)phenyl)carbamate (Compound 30)

### Step 1: Preparation of tert-butyl 4-(4-(((oxazol-5-ylmethoxy)carbonyl)amino)benzyl)piperidine-1l-carboxylate

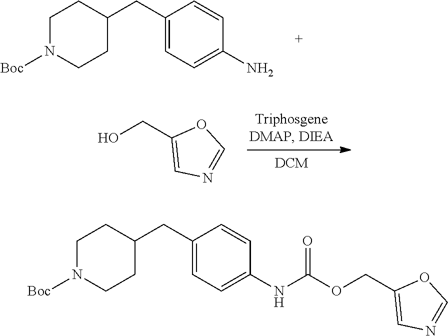 [SMILES: CC(C)(C)OC(=O)N1CCC(Cc2ccc(N)cc2)CC1 ; OCc1cnco1 ; CC(C)(C)OC(=O)N1CCC(Cc2ccc(NC(=O)OCc3cnco3)cc2)CC1]

To a solution of triphosgene (2.38 g, 8.0 mmol) in DCM (100 mL) at 0° C. was added a mixture of tert-butyl 4-(4-aminobenzyl)piperidine-1-carboxylate (5.81 g, 20.0 mmol), DIEA (5.68 g, 44.0 mmol), DMAP (2.22 g, 20.0 mmol) in DCM (100 mL). The mixture was stirred at 0° C. for 5 min and oxazol-5-ylmethanol (2.58 g, 26.0 mmol) was added. The resulting mixture was stirred at 24° C. for 1 h and concentrated. The residue was purified by silica gel chromatography eluted with EtOAc/Hexane (1:1) to afford the desired product (7.1 g, 85.4%) as a white solid. LRMS (ES) 416.1 [M+H]⁺.

### Step 2: Preparation of oxazol-5-ylmethyl (4-(piperidin-4-ylmethyl)phenyl)carbamate hydrochloride

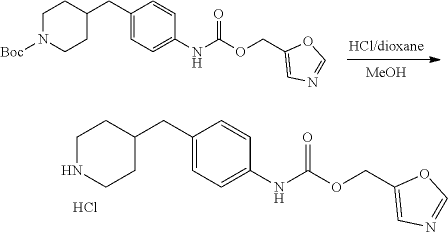 [SMILES: "MeOH" ; CC(C)(C)OC(=O)N1CCC(Cc2ccc(NC(=O)OCc3cnco3)cc2)CC1 ; O=C(Nc1ccc(CC2CCNCC2)cc1)OCc1cnco1 ; Cl]

To a mixture of tert-butyl 4-(4-(((oxazol-5-ylmethoxy)carbonyl)amino)benzyl)piperidine-1-carboxylate (7.1 g, 17.1 mmol) in MeOH (10 mL) was added 4M HCl/dioxane (36 mL, 144 mmol). The reaction mixture was stirred at 24° C. for 2 h and concentrated to dryness to afford the crude product as a white solid (6.01 g, 100%). LRMS (m/z): 316.1 [M+H]⁺.

### Step 3: Preparation of oxazol-5-ylmethyl (4-((1-(dimethylcarbamoyl)piperidin-4-yl)methyl)phenyl)carbamate

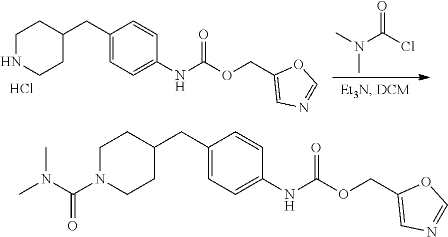

To a mixture of oxazol-5-ylmethyl (4-(piperidin-4-ylmethyl)phenyl)carbamate hydrochloride (1.97 g, 5.6 mmol), and DIEA (1.81 g, 14.0 mmol) in DCM at 0° C. was added dimethylcarbamoyl chloride (0.66 g, 6.16 mmol) dropwise. The reaction mixture was warmed to 24° C. and stirred for 1 h. The mixture was concentrated and the residue was purified by silica gel chromatography eluted with EtOAc to afford the desired product (1.66 g, 76.7%) as a white solid. LRMS (m/z): 387.1 [M+H]⁺. ¹H NMR (400 MHz, Methanol-d₄) δ 8.24 (s, 1H), 7.35 (d, J=8.0 Hz, 2H), 7.25 (s, 1H), 7.18-6.98 (m, 2H), 5.24 (s, 2H), 3.64 (d, J=13.0 Hz, 2H), 2.82 (d, J=1.4 Hz, 6H), 2.72 (td, J=12.8, 2.3 Hz, 2H), 2.52 (d, J=6.9 Hz, 2H), 1.76-1.56 (m, 3H), 1.28-1.12 (m, 2H).

### Example S

### Preparation of oxazol-5-ylmethyl (R)-(4-(1-(dimethylcarbamoyl)piperidin-3-yl)phenyl)carbamate (Compound 376)

### Step 1: Preparation of tert-butyl (R)-3-(4-(((oxazol-5-ylmethoxy)carbonyl)amino)phenyl)piperidine-1-carboxylate

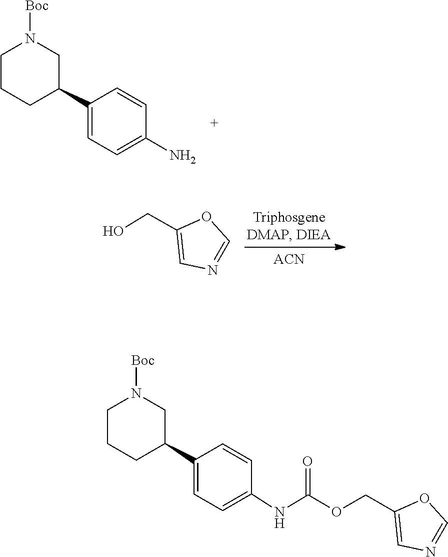

To a mixture of triphosgene (0.715 g, 2.41 mmol) in 60 mL acetonitrile at 0° C. was added a mixture of tert-butyl (R)-3-(4-aminophenyl)piperidine-1-carboxylate (2.00 g, 7.24 mmol) and N,N-diisopropylethylamine (1.87 g, 14.47 mmol) in 40 mL MeCN. The mixture was stirred at 0° C. for 15 min, warmed to 24° C., followed by addition of a mixture of DMAP (44 mg, 0.362 mmol, 0.05 equiv) and 1,3-oxazol-5-ylmethanol (790 mg, 8.00 mmol) in MeCN (10 mL). The mixture was stirred at 24° C. for 16 h and at 50° C. for 1 h. The reaction mixture was cooled and concentrated. The mixture was dissolved in 150 mL dichloromethane, washed with 0.5 N HCl, brine, dried (Na₂SO₄) and concentrated to afford the crude product (2.91 g) as an off-white foam that was used in the next reaction without further purification. LRMS (ES) 402.2 [M+H]⁺.

### Step 2: Preparation of oxazol-5-ylmethyl (R)-(4-(piperidin-3-yl)phenyl)carbamate hydrochloride

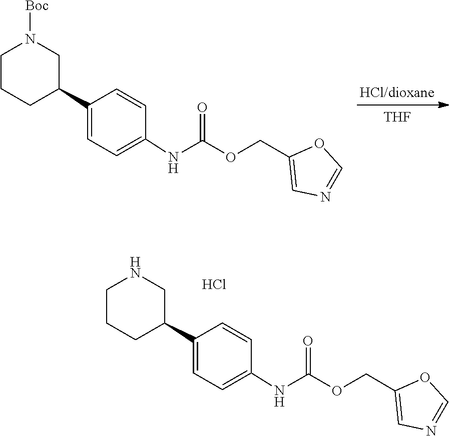 [SMILES: CC(C)(C)OC(=O)N1CCC[C@H](c2ccc(NC(=O)OCc3cnco3)cc2)C1 ; O=C(Nc1ccc([C@H]2CCCNC2)cc1)OCc1cnco1 ; Cl ; "THF"]

To a mixture of tert-butyl (R)-3-(4-(((oxazol-5-ylmethoxy)carbonyl)amino)phenyl)piperidine-1-carboxylate (2.90 g, 7.20 mmol) in THF (36 mL) was added 4M HCl/dioxane (36 mL, 144 mmol). The reaction mixture was stirred at 24° C. for 16 h, filtered, washed with EtOAc (2×30 mL) to give the crude product as an off-white solid in quantitative yield. LRMS (m/z): 302.1 [M+H]⁺.

### Step 3: Preparation of oxazol-5-ylmethyl (R)-(4-(1-(dimethylcarbamoyl)piperidin-3-yl)phenyl)carbamate

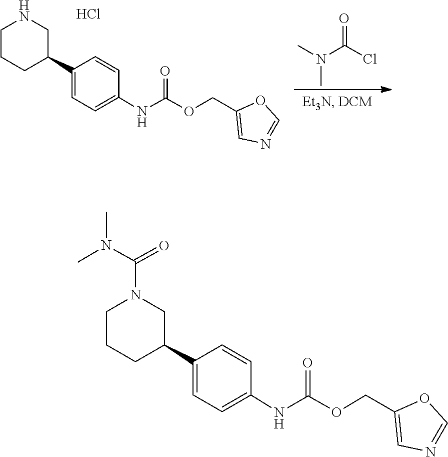

To a mixture of (3R)-3-(4-{[(1,3-oxazol-5-ylmethoxy)carbonyl]amino}phenyl)piperidin-1-ium chloride (243 mg, 0.72 mmol, 1 equiv), N,N-diisopropylethylamine (0.5 mL, 2.88 mmol, 4 equiv) and DCM (5 mL) was added dimethylcarbamyl chloride (1.17 mmol) dropwise, the mixture was stirred at 24° C. for 2 h, diluted with 5 mL DCM, washed with 0.5 N HCl, saturated NaHCO₃, and brine, dried (Na₂SO₄), and concentrated. The residue was purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford oxazol-5-ylmethyl (R)-(4-(1-(dimethylcarbamoyl)piperidin-3-yl)phenyl)carbamate (0.15 g, 56%) as a white solid. LRMS (m/z): 373.1 [M+H]⁺. ¹H NMR (400 MHz, DMSO-d₆) δ 9.76 (s, 1H), 8.43 (s, 1H), 7.38 (d, J=8.1 Hz, 2H), 7.31 (s, 1H), 7.18 (d, J=8.5 Hz, 2H), 5.21 (s, 2H), 3.55 (dd, J=22.5, 12.0 Hz, 2H), 2.73 (s, 6H), 2.74-2.66 (m, 2H), 2.64 (d, J=12.0 Hz, 1H), 1.87 (d, J=11.0 Hz, 1H),

### Example T

### Preparation of oxazol-5-ylmethyl (4-(1-isobutyrylpiperidin-4-yl)phenyl)carbamate (Compound 7)

### Step 1: Preparation of tert-butyl 4-(4-(((oxazol-5-ylmethoxy)carbonyl)amino)phenyl)piperidine-1-carboxylate

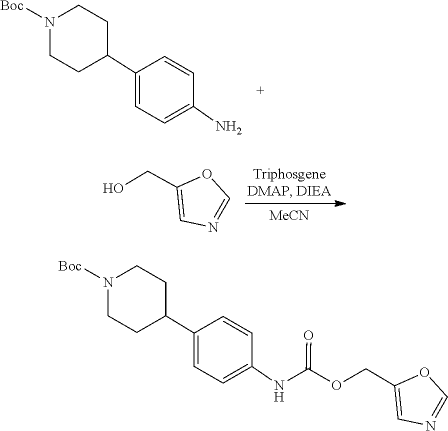 [SMILES: "MeCN" ; CC(C)(C)OC(=O)N1CCC(c2ccc(N)cc2)CC1 ; OCc1cnco1 ; CC(C)(C)OC(=O)N1CCC(c2ccc(NC(=O)OCc3cnco3)cc2)CC1]

To a mixture of triphosgene (1.48 g, 5.00 mmol) in 150 mL of acetonitrile at 0° C. was added a mixture of tert-butyl 4-(4-aminophenyl)piperidine-1-carboxylate (4.15 g, 15.0 mmol) and N,N-diisopropylethylamine (3.88 g, 30.0 mmol) in 100 mL acetonitrile The mixture was stirred at 0° C. for 15 min, then warmed to 24° C., followed by addition of a mixture of DMAP (92 mg, 0.75 mmol) and 1,3-oxazol-5-ylmethanol (1.82 g, 18.0 mmol) in MeCN (25 mL). The mixture was stirred at 24° C. for 16 h, then at 50° C. for 1 h. then concentrated. The mixture was dissolved in 350 mL dichloromethane, washed with 0.5 N HCl, brine, dried (Na₂SO₄) and concentrated to afford the crude product (6.00 g) as an off-white foam that was used in the next reaction without further purification. LRMS (ES) 402.2 [M+H]⁺.

### Step 2: Preparation of oxazol-5-ylmethyl (4-(piperidin-4-yl)phenyl)carbamate hydrochloride

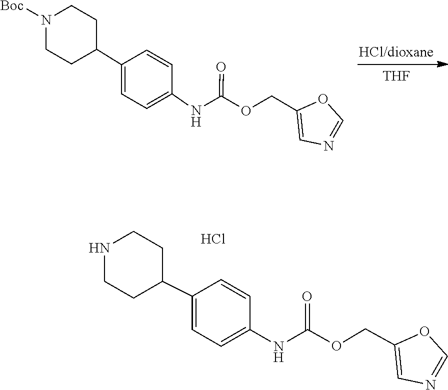 [SMILES: CC(C)(C)OC(=O)N1CCC(c2ccc(NC(=O)OCc3cnco3)cc2)CC1 ; O=C(Nc1ccc(C2CCNCC2)cc1)OCc1cnco1 ; Cl ; "THF"]

To a mixture of tert-butyl 4-(4-(((oxazol-5-ylmethoxy)carbonyl)amino)phenyl)piperidine-1-carboxylate (5.80 g, 14.4 mmol) in THF (72 mL) was added 4M HCl/dioxane (72 mL, 288 mmol). The reaction mixture was stirred at 24° C. for 16 h, filtered, and washed with EtOAc (2×60 mL) to give the desired crude product which was obtained as an off-white solid in quantitative yield. LRMS (m/z): 302.1 [M+H]⁺.

### Step 3: Preparation of oxazol-5-ylmethyl (4-(1-isobutyrylpiperidin-4-yl)phenyl)carbamate

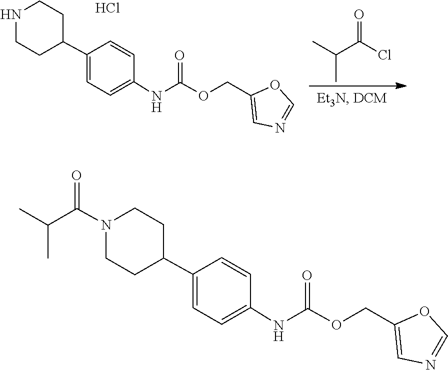

To a mixture of oxazol-5-ylmethyl (4-(piperidin-4-yl)phenyl)carbamate hydrochloride (0.74 mmol) and N,N-diisopropylethylamine (05 mL, 2.88 mmol) in dichloromethane (5 mL) was added isobutyl chloride (0.095 g, 0.89 mmol) dropwise. The mixture was allowed to stir at 24° C. for 2 h. diluted with 5 mL DCM, washed with 0.5 N HCl, saturated NaHCO₃, and brine respectively, dried (Na₂SO₄), and concentrated. The residue was purified by Prep-HPLC with the following conditions (Column: XBridge Prep OBD C18 Column 30×150 mm 5 um; Mobile Phase A:Water (0.1% formic acid), Mobile Phase B: MeCN (0.1% formic acid; Flow rate: 20 mL/min) to afford oxazol-5-ylmethyl (4-(1-isobutyrylpiperidin-4-yl)phenyl)carbamate (0.15 g, 55%) as a white solid. LRMS (m/z): 372.1 [M+H]⁺. ¹H NMR (400 MHz, DMSO-d₆) δ 9.73 (s, 1H), 8.42 (s, 1H), 7.37 (d, J=8.1 Hz, 2H), 7.30 (s, 1H), 7.16 (d, J=8.5 Hz, 2H), 5.21 (s, 2H), 4.55 (d, J=12.8 Hz, 1H), 4.05 (d, J=13.4 Hz, 1H), 3.09 (t, J=12.9 Hz, 1H), 2.90 (p, J=6.7 Hz, 1H), 2.69 (d, J=12.1 Hz, 1H), 2.58 (d, J=12.7 Hz, 1H), 1.78 (t, J=17.1 Hz, 2H), 1.52-1.34 (m, 2H), 1.01 (d, J=6.6 Hz, 6H).

### Synthesis of Comparator Ureas

### Example U

### Preparation of 1-(4-chlorobenzyl)-3-(4-((1-(oxetan-3-yl)piperidin-4-yl)methyl)phenyl)urea (Comparator 1)

### Step 1: Preparation of tert-butyl 4-(4-(3-(4-chlorobenzyl)ureido)benzyl)piperidine-1-carboxylate

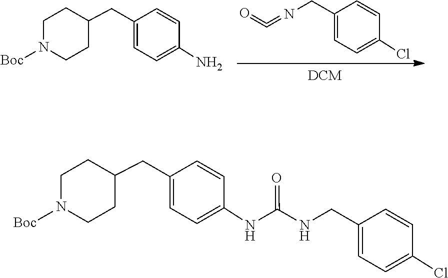 [SMILES: CC(C)(C)OC(=O)N1CCC(Cc2ccc(N)cc2)CC1 ; O=C=NCc1ccc(Cl)cc1 ; CC(C)(C)OC(=O)N1CCC(Cc2ccc(NC(=O)NCc3ccc(Cl)cc3)cc2)CC1]

A mixture of tert-butyl 4-(4-aminobenzyl)piperidine-1-carboxylate (0.55 g, 1 equiv) and 1-chloro-4-(isocyanatomethyl)benzene (1.5 equiv) in DCM (3 mL) was stirred at 24° C. for 1 h, and purified by silica gel chromatography (50% EtOAc/Hexanes) to afford tert-butyl 4-(4-(3-(4-chlorobenzyl)ureido)benzyl)piperidine-1-carboxylate (0.84 g, 96.8% yield). LRMS (ES) 403.1 288.1 [M+H-Bu]⁺.

### Step 2: Preparation of 1-(4-chlorobenzyl)-3-(4-(piperidin-4-ylmethyl)phenyl)urea hydrochloride

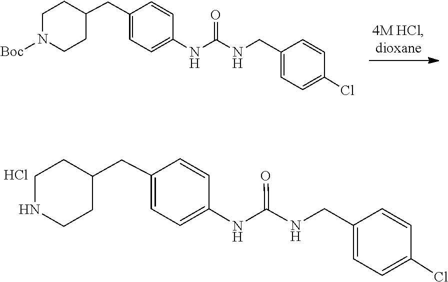 [SMILES: CC(C)(C)OC(=O)N1CCC(Cc2ccc(NC(=O)NCc3ccc(Cl)cc3)cc2)CC1 ; Cl ; O=C(NCc1ccc(Cl)cc1)Nc1ccc(CC2CCNCC2)cc1]

To a mixture of tert-butyl 4-(4-(3-(4-chlorobenzyl)ureido)benzyl)piperidine-1-carboxylate (0.84 g, 1 equiv) in MeOH (1 mL) at 0° C., was added 4 M HCl in dioxane (12 equiv), the mixture was stirred at 24° C. for 1 h and concentrated to afford 1-(4-chlorobenzyl)-3-(4-(piperidin-4-ylmethyl)phenyl)urea hydrochloride (0.72 g, 99.6% yield) that was used in the next reaction without further purification. LRMS (ES) 358.1 [M+H].

equivequiv

### Step 3: Preparation of 1-(4-chlorobenzyl)-3-(4-((1-(oxetan-3-yl)piperidin-4-yl)methyl)phenyl)urea

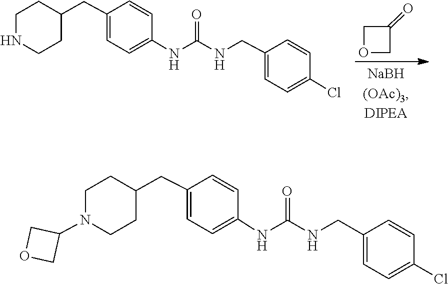 [SMILES: O=C(NCc1ccc(Cl)cc1)Nc1ccc(CC2CCNCC2)cc1 ; O=C1COC1 ; O=C(NCc1ccc(Cl)cc1)Nc1ccc(CC2CCN(C3COC3)CC2)cc1]

To a mixture of 1-(4-chlorobenzyl)-3-(4-(piperidin-4-ylmethyl)phenyl)urea hydrochloride (0.47 g, 1 equiv) and oxetan-3-one (2 equiv) in DCM (3 mL) was added Na(OAc)₃BH (2.2 equiv) and DIPEA (1 equiv). The reaction mixture was stirred at 24° C. for 16 hours, quenched with saturated NaHCO₃(5 mL), extracted with DCM (5 mL) twice. equivequivequivequiv The organic layer was combined, concentrated, and purified on HPLC (10% AcCN) to afford 1-(4-chlorobenzyl)-3-(4-((1-(oxetan-3-yl)piperidin-4-yl)methyl)phenyl)urea (0.27 g, 53.3% yield). LRMS (ES) 414.1 [M+H]. ¹H NMR (400 MHz, DMSO-d₆) δ 8.48 (s, 1H), 7.39 (d, J=8.5 Hz, 2H), 7.31 (d, J=8.4 Hz, 2H), 7.29 (d, J=8.3 Hz, 2H), 7.00 (d, J=8.3 Hz, 2H), 6.59 (t, J=6.0 Hz, 1H), 4.49 (t, J=6.4 Hz, 2H), 4.38 (t, J=6.1 Hz, 2H), 4.27 (d, J=6.0 Hz, 2H), 3.30 (p, J=6.5 Hz, 1H), 2.63 (d, J=11.0 Hz, 2H), 2.41 (d, J=6.9 Hz, 2H), 1.64 (t, J=11.2 Hz, 2H), 1.53 (d, J=11.4 Hz, 2H), 1.48-1.35 (m, 1H), 1.15 (qd, J=12.1, 3.7 Hz, 2H).

Compounds in the following table were prepared in a similar manner as Comparator 1, using the intermediates with alkylation/acylation reagents and methods as listed.

| | Alkylation/ | |
|---|---|---|
| | acylation | |
| Aniline | Reagent | Structure, Name and Data |
| 4-(4- aminobenzyl)piperidine- 1-carboxylate | acetic anhydride | 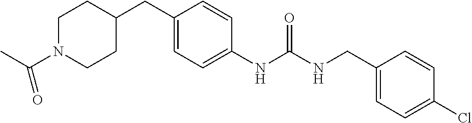 [SMILES: CC(=O)N1CCC(Cc2ccc(NC(=O)NCc3ccc(Cl)cc3)cc2)CC1] Comparator 2: 1-(4-((1-acetylpiperidin-4- yl)methyl)phenyl)-3-(4-chlorobenzyl)urea (0.23 g, 71.9% yield). LRMS (ES) 400.1 [M + H]. ¹H NMR (400 MHz, Methanol-d₄) δ 7.42-7.20 (m, 6H), 7.19-7.00 (m, 2H), 4.49 (d, J = 13.1 Hz, 1H), 4.38 (s, 2H), 3.89 (d, J = 13.8 Hz, 1H), 3.05 (t, J = 13.3 Hz, 1H), 2.64- 2.44 (m, 3H), 2.09 (s, 3H), 1.88-1.62 (m, 3H), 1.26- 1.02 (m, 2H). |
| tert-butyl 4-((4- aminobenzyl)oxy) piperidine- 1-carboxylate | oxetan-3-one | 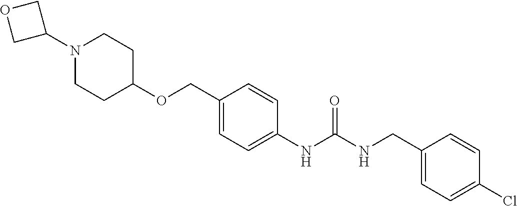 [SMILES: O=C(NCc1ccc(Cl)cc1)Nc1ccc(COC2CCN(C3COC3)CC2)cc1] Comparator 3: 1-(4-chlorobenzy1)-3-(4-(((1- (oxetan-3-yl)piperidin-4-yl)oxy)methyl)phenyl)urea. LRMS (ES) 430.1 [M + H], ¹H NMR (400 MHZ, Methanol-d₄) δ 7.39 (d, J = 8.2 Hz, 2H), 7.32 (s, 4H), 7.27 (d, J = 8.2 Hz, 2H), 4.88-4.72 (m, 4H), 4.49 (s, 2H), 4.37 (s, 2H), 4.24 (p, J = 6.6 Hz, 1H), 3.73 (tt, J = 6.1, 3.3 Hz, 1H), 3.20-3.04 (m, 2H), 3.04-2.89 (m, 2H), 2.19-1.61 (m, 4H). |

### Example V

### Preparation of 1-(oxazol-5-ylmethyl)-3-(4-((1-(oxetan-3-yl)piperidin-4-yl)methyl)phenyl)urea (Comparator 4)

### Step 1: Preparation of tert-butyl 4-(4-isocyanatobenzyl)piperidine-1-carboxylate

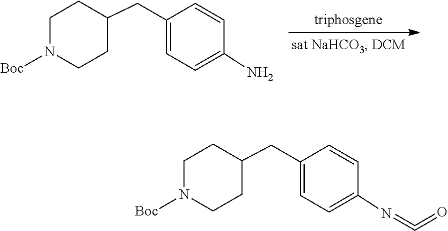 [SMILES: CC(C)(C)OC(=O)N1CCC(Cc2ccc(N)cc2)CC1 ; CC(C)(C)OC(=O)N1CCC(Cc2ccc(N=C=O)cc2)CC1]

A mixture of tert-butyl 4-(4-aminobenzyl)piperidine-1-carboxylate (2.4 g, 1 equiv) and sat. NaHCO₃ in DCM (50 mL) was stirred at 0° C. Triphosgene (0.33 equiv) in DCM (10 mL) was added and the mixture was stirred for 15 minutes at 0° C. The organic layer was dried to afford tert-butyl 4-(4-isocyanatobenzyl)piperidine-1-carboxylate and was used in the next reaction without further purification.

### Step 2: Preparation of tert-butyl 4-(4-(3-(oxazol-5-ylmethyl)ureido)benzyl)piperidine-1-carboxylate

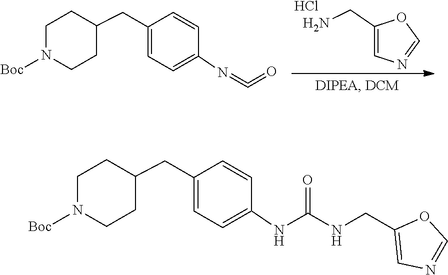 [SMILES: Cl ; CC(C)(C)OC(=O)N1CCC(Cc2ccc(N=C=O)cc2)CC1 ; NCc1cnco1 ; CC(C)(C)OC(=O)N1CCC(Cc2ccc(NC(=O)NCc3cnco3)cc2)CC1]

To a mixture of tert-butyl 4-(4-isocyanatobenzyl)piperidine-1-carboxylate (2.6 g, 1 equiv) in DCM was added oxazol-5-ylmethanamine hydrochloride (1.5 equiv) and DIPEA (1.5 equiv). The mixture was stirred at 24° C. for 1 h, washed with 1 N HCl, brine and dried over Na₂SO₄ and concentrated to afford tert-butyl 4-(4-(3-(oxazol-5-ylmethyl)ureido)benzyl)piperidine-1-carboxylate. LRMS (ES) 359.1 [M+H].

### Step 3: Preparation of 1-(oxazol-5-ylmethyl)-3-(4-(piperidin-4-ylmethyl)phenyl)urea hydrochloride

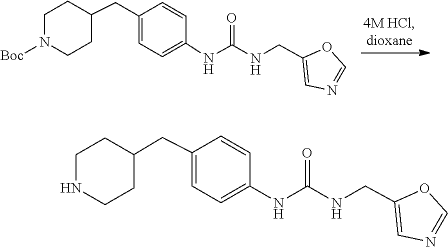 [SMILES: CC(C)(C)OC(=O)N1CCC(Cc2ccc(NC(=O)NCc3cnco3)cc2)CC1 ; O=C(NCc1cnco1)Nc1ccc(CC2CCNCC2)cc1]

A mixture of tert-butyl 4-(4-(3-(oxazol-5-ylmethyl)ureido)benzyl)piperidine-1-carboxylate in MeOH was cooled to 0° C., followed by the addition of 4 M HCl in dioxane (12 equiv), stirring at 24° C. for 1 h, and concentration to afford 1-(oxazol-5-ylmethyl)-3-(4-(piperidin-4-ylmethyl)phenyl)urea hydrochloride and was used in the next reaction without further purification. LRMS (ES) 315.1 [M+H].

### Step 4: Preparation of 1-(oxazol-5-ylmethyl)-3-(4-((1-(oxetan-3-yl)piperidin-4-yl)methyl)phenyl)urea

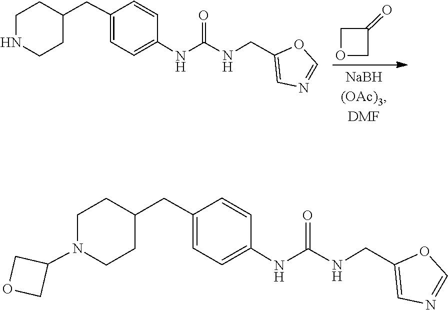

A mixture of 1-(oxazol-5-ylmethyl)-3-(4-(piperidin-4-ylmethyl)phenyl)urea hydrochloride (150 mg, 1 equiv) and oxetan-3-one (1 equiv) in DMF (1 mL) was stirred at 24° C. for 20 minutes. Sodium triacetoxyborohydride (2.2 equiv) was added and the mixture was stirred for 1 h. The mixture was filtered and purified by prep HPLC to afford 1-(oxazol-5-ylmethyl)-3-(4-((1-(oxetan-3-yl)piperidin-4-yl)methyl)phenyl)urea (49 mg, 31% yield). LRMS (ES) 371.1 [M+H]. ¹H NMR (400 MHz, DMSO-d₆) δ 8.46 (s, 1H), 7.27 (d, J=8.0 Hz, 2H), 7.00 (d, J=7.7 Hz, 3H), 6.55 (t, J=5.9 Hz, 1H), 4.48 (t, J=6.5 Hz, 2H), 4.43-4.29 (m, 4H), 3.29 (d, J=7.5 Hz, 1H), 2.62 (d, J=11.1 Hz, 2H), 2.41 (d, J=6.9 Hz, 2H), 1.64 (t, J=11.4 Hz, 2H), 1.52 (d, J=12.9 Hz, 2H), 1.41 (s, 1H), 1.16 (t, J=11.9 Hz, 2H).

Compounds in the following table were prepared in a similar manner as Comparator 4, using the intermediates with alkylation/acylation reagents and methods as listed.

| | Amine capped | |
|---|---|---|
| Aniline | reagent | Structure, Name and Data |
| 4-(4- aminobenzyl)piperidine- 1-carboxylate | acetic anhydride | 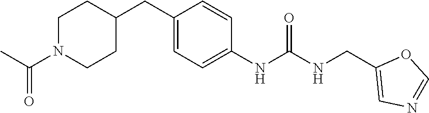 [SMILES: CC(=O)N1CCC(Cc2ccc(NC(=O)NCc3cnco3)cc2)CC1] Comparator 5: 1-(4-((1-acetylpiperidin-4- yl)methyl)phenyl)-3-(oxazol-5- ylmethyl)urea. LRMS (ES) 357.1 [M + H]. ¹H NMR (400 MHZ, DMSO- d₆) δ 8.46 (s, 1H), 8.28 (s, 1H), 7.29 (d, J = 7.8 Hz, 2H), 7.05-6.96 (m, 3H), 6.55 (s, 1H), 4.40-4.27 (m, 3H), 3.75 (d, J = 13.6 Hz, 1H), 2.92 (t, J = 12.9 Hz, 1H), 2.43 (t, J = 9.2 Hz, 3H), 1.95 (s, 3H), 1.67 (s, 1H), 1.55 (t, J = 12.8 Hz, 2H), 1.01 (dt, J = 50.0, 12.6 Hz, 2H). |
| 4-(4- aminobenzyl)piperidine- 1-carboxylate | dimethylcarbamoyl chloride | 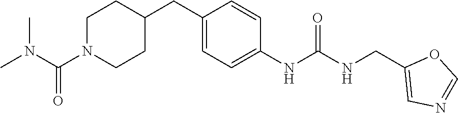 [SMILES: CN(C)C(=O)N1CCC(Cc2ccc(NC(=O)NCc3cnco3)cc2)CC1] Comparator 6: N,N-dimethyl-4-(4-(3-(oxazol-5- ylmethyl)ureido)benzyl)piperidine- 1-carboxamide. LRMS (ES) 386.1 [M + H]. ¹H NMR (400 MHZ, DMSO- d₆) δ 8.46 (s, 1H), 8.27 (s, 1H), 7.28 (d, J = 8.0 Hz, 2H), 7.00 (d, J = 7.9, 4.8 Hz, 3H), 6.57 (s, 1H), 4.35 (d, J = 5.8 Hz, 2H), 3.49 (d, J = 13.3 Hz, 2H), 2.69 (s, 6H), 2.58 (t, J = 12.3 Hz, 2H), 2.42 (d, J = 6.9 Hz, 2H), 1.69-1.47 (m, 3H), 1.08 (q, J = 12.1 Hz, 2H). |

### Biological Example 1

### NMN Fluorescence Biochemical Assay

### Human Recombinant Enzyme Assay

Compounds described herein were assayed for their ability to stimulate the synthesis of nicotinamide mononucleotide (NMN) by the enzyme NAMPT. The human recombinant enzyme assay measures the activation of the enzyme activity by compounds using recombinant enzyme and substrates in a buffered cell-free system. The assay conditions closely mimic cellular environments. Dose responses were measured using an assay to detect the formation of nicotinamide mono-nucleotide. All experiments were performed in the 384-well format. Generally, 0.5 μL of DMSO containing varying concentrations of the test compound was mixed with 10 μL of the enzyme reagent solution. Enzyme reactions were initiated with the addition of L of a solution containing the substrates. The final assay conditions were as follows: 6 nM human NAMPT, 2.5 mM ATP, 20 μM PRPP and 150 μM nicotinamide in 50 mM HEPES, pH 7.2, 1 mM DTT, 1 mM CHAPS 50 mM NaCl, 100 mM MgCl₂. Following an incubation of 60 min at ambient temperature, 10 μL of 20% acetophenone in DMSO was added, followed by 10 μL of 2 M KOH and 40 μL of formic acid. The plates were read for fluorescence (Excitation/Emission=355 nm/460 nm) using an EnVision plate reader after 40 mins of incubation at ambient temperature. The potency measurements for compounds are quantified and represented as AC_{1.4} (the concentration of compounds that generates 40% higher activity over basal) and EC₅₀ (concentration of the compound that gives half-maximal activation). Table A shows the AC_{1.4} and EC₅₀ data and for the tested compounds.

TABLE A

| Compound | AC_{1.4} | EC₅₀ |
|---|---|---|
| No. | Human (μM) | Human (μM) |
| 1 | 0.03 | 0.44 |
| 2 | 0.05 | 0.96 |
| 3 | 0.05 | 0.31 |
| 4 | 0.06 | 21.81 |
| 5 | 0.07 | 0.29 |
| 6 | 0.07 | 0.55 |
| 7 | 0.11 | 20.81 |
| 8 | 0.14 | 32.56 |
| 9 | 0.15 | 16.00 |
| 10 | 0.16 | 11.19 |
| 11 | 0.21 | 22.51 |
| 12 | 0.22 | 0.34 |
| 13 | 0.22 | 44.66 |
| 14 | 0.23 | 10.74 |
| 15 | 0.24 | 2.16 |
| 16 | 0.25 | 3.50 |
| 17 | 0.25 | 0.90 |
| 18 | 0.25 | 16.18 |
| 19 | 0.26 | 0.59 |
| 20 | 0.29 | 2.57 |
| 21 | 0.30 | 2.00 |
| 22 | 0.32 | 10.08 |
| 23 | 0.32 | 2.76 |
| 24 | 0.34 | 1.88 |
| 25 | 0.35 | 7.80 |
| 26 | 0.35 | 27.47 |
| 27 | 0.36 | 5.25 |
| 28 | 0.36 | 3.01 |
| 29 | 0.37 | 1.51 |
| 30 | 0.37 | 25.14 |
| 31 | 0.37 | 21.68 |
| 32 | 0.38 | 20.97 |
| 33 | 0.38 | 1.01 |
| 34 | 0.38 | 0.82 |
| 35 | 0.39 | 37.92 |
| 36 | 0.40 | 1.00 |
| 37 | 0.41 | 3.67 |
| 38 | 0.42 | 1.22 |
| 39 | 0.43 | 19.17 |
| 40 | 0.43 | 2.40 |
| 41 | 0.44 | 3.48 |
| 42 | 0.44 | 5.66 |
| 43 | 0.45 | 4.56 |
| 44 | 0.45 | 4.19 |
| 45 | 0.46 | 1.88 |
| 46 | 0.48 | 2.58 |
| 47 | 0.49 | 9.61 |
| 48 | 0.49 | 31.01 |
| 49 | 0.50 | 2.02 |
| 50 | 0.50 | 0.89 |
| 51 | 0.50 | 40.31 |
| 52 | 0.50 | 5.38 |
| 53 | 0.51 | 5.89 |
| 54 | 0.51 | 8.45 |
| 55 | 0.51 | 1.82 |
| 56 | 0.53 | 2.80 |
| 57 | 0.54 | 1.29 |
| 58 | 0.55 | 1.73 |
| 59 | 0.56 | 48.93 |
| 60 | 0.57 | 1.91 |
| 61 | 0.57 | 2.62 |
| 62 | 0.57 | 2.64 |
| 63 | 0.57 | 4.93 |
| 64 | 0.58 | 4.22 |
| 65 | 0.58 | 37.12 |
| 66 | 0.59 | 5.12 |
| 67 | 0.59 | 21.33 |
| 68 | 0.59 | 11.05 |
| 69 | 0.59 | 3.07 |
| 70 | 0.61 | 22.95 |
| 71 | 0.61 | 2.51 |
| 72 | 0.62 | 5.27 |
| 73 | 0.62 | 1.76 |
| 74 | 0.64 | 1.42 |
| 75 | 0.64 | 26.31 |
| 76 | 0.64 | 0.89 |
| 77 | 0.65 | 1.67 |
| 78 | 0.65 | 7.44 |
| 79 | 0.66 | 23.59 |
| 80 | 0.66 | 28.98 |
| 81 | 0.66 | 3.41 |
| 82 | 0.66 | 3.90 |
| 83 | 0.66 | 8.11 |
| 84 | 0.66 | 3.12 |
| 85 | 0.67 | 6.38 |
| 86 | 0.68 | 6.21 |
| 87 | 0.68 | 1.75 |
| 88 | 0.69 | 37.44 |
| 89 | 0.71 | 9.40 |
| 90 | 0.72 | 7.32 |
| 91 | 0.74 | 3.80 |
| 92 | 0.75 | 3.22 |
| 93 | 0.75 | 6.80 |
| 94 | 0.75 | 2.36 |
| 95 | 0.75 | 3.11 |
| 96 | 0.75 | 3.47 |
| 97 | 0.76 | 23.61 |
| 98 | 0.78 | 1.49 |
| 99 | 0.79 | 51.18 |
| 100 | 0.81 | 1.22 |
| 101 | 0.82 | 4.14 |
| 102 | 0.82 | 1.89 |
| 103 | 0.83 | 4.31 |
| 104 | 0.85 | 5.06 |
| 105 | 0.85 | 1.94 |
| 106 | 0.86 | 26.38 |
| 107 | 0.89 | 10.81 |
| 108 | 0.89 | 8.17 |
| 109 | 0.90 | 4.71 |
| 110 | 0.91 | 3.46 |
| 111 | 0.91 | 5.33 |
| 112 | 0.93 | 5.53 |
| 113 | 0.94 | 50.96 |
| 114 | 0.94 | 19.66 |
| 115 | 0.94 | 3.13 |
| 116 | 0.95 | 6.77 |
| 117 | 0.95 | 8.14 |
| 118 | 0.95 | 3.13 |
| 119 | 0.95 | 21.72 |
| 120 | 0.96 | 44.85 |
| 121 | 0.97 | 14.49 |
| 122 | 0.97 | 6.46 |
| 123 | 0.98 | 5.01 |
| 124 | 0.98 | 15.29 |
| 125 | 0.98 | 6.02 |
| 126 | 0.99 | 4.06 |
| 127 | 1.00 | 7.30 |
| 128 | 1.03 | 4.75 |
| 129 | 1.03 | 2.22 |
| 130 | 1.03 | 8.13 |
| 131 | 1.05 | 2.55 |
| 132 | 1.06 | 41.14 |
| 133 | 1.07 | 8.60 |
| 134 | 1.07 | 13.74 |
| 135 | 1.07 | 3.28 |
| 136 | 1.09 | 2.23 |
| 137 | 1.11 | 3.83 |
| 138 | 1.11 | 8.41 |
| 139 | 1.11 | 13.06 |
| 140 | 1.15 | 4.51 |
| 141 | 1.17 | 2.24 |
| 142 | 1.17 | 7.35 |
| 143 | 1.18 | 10.78 |
| 144 | 1.18 | 7.67 |
| 145 | 1.19 | 8.23 |
| 146 | 1.20 | 2.35 |
| 147 | 1.22 | 5.21 |
| 148 | 1.23 | 11.53 |
| 149 | 1.23 | 7.00 |
| 150 | 1.24 | 4.31 |
| 151 | 1.25 | 5.89 |
| 152 | 1.27 | 10.58 |
| 153 | 1.27 | 33.82 |
| 154 | 1.30 | 11.95 |
| 155 | 1.31 | 5.80 |
| 156 | 1.31 | 9.45 |
| 157 | 1.32 | 9.11 |
| 158 | 1.32 | 1.27 |
| 159 | 1.33 | 4.30 |
| 160 | 1.33 | 5.32 |
| 161 | 1.33 | 10.11 |
| 162 | 1.36 | 4.50 |
| 163 | 1.37 | 0.68 |
| 164 | 1.38 | 1.60 |
| 165 | 1.38 | 7.53 |
| 166 | 1.38 | 5.87 |
| 167 | 1.39 | 4.54 |
| 168 | 1.40 | 13.86 |
| 169 | 1.41 | 6.11 |
| 170 | 1.41 | 9.33 |
| 171 | 1.43 | 9.75 |
| 172 | 1.43 | 3.54 |
| 173 | 1.46 | 4.39 |
| 174 | 1.47 | 28.00 |
| 175 | 1.47 | 8.92 |
| 176 | 1.47 | 4.56 |
| 177 | 1.49 | 38.27 |
| 178 | 1.50 | 14.72 |
| 179 | 1.50 | 22.42 |
| 180 | 1.52 | 15.13 |
| 181 | 1.53 | 15.60 |
| 182 | 1.54 | 10.95 |
| 183 | 1.57 | 15.15 |
| 184 | 1.58 | 14.88 |
| 185 | 1.59 | 2.04 |
| 186 | 1.61 | 3.38 |
| 187 | 1.62 | 11.25 |
| 188 | 1.62 | 6.85 |
| 189 | 1.64 | 18.62 |
| 190 | 1.64 | 9.00 |
| 191 | 1.69 | 21.95 |
| 192 | 1.70 | 6.45 |
| 193 | 1.71 | 12.71 |
| 194 | 1.73 | 26.66 |
| 195 | 1.73 | 8.13 |
| 196 | 1.75 | 7.22 |
| 197 | 1.75 | 11.77 |
| 198 | 1.76 | 8.59 |
| 199 | 1.77 | 5.28 |
| 200 | 1.78 | 2.12 |
| 201 | 1.78 | 26.42 |
| 202 | 1.82 | 24.46 |
| 203 | 1.82 | 7.14 |
| 204 | 1.83 | 21.14 |
| 205 | 1.84 | 10.06 |
| 206 | 1.85 | 14.65 |
| 207 | 1.85 | 9.34 |
| 208 | 1.87 | 17.36 |
| 209 | 1.88 | 21.61 |
| 210 | 1.91 | 3.82 |
| 211 | 1.91 | 10.57 |
| 212 | 1.93 | 10.19 |
| 213 | 1.95 | 2.92 |
| 214 | 1.96 | 9.59 |
| 215 | 1.96 | 8.35 |
| 216 | 1.98 | 5.81 |
| 217 | 1.98 | 24.43 |
| 218 | 1.99 | 9.06 |
| 219 | 2.03 | 20.58 |
| 220 | 2.03 | 32.47 |
| 221 | 2.04 | 2.16 |
| 222 | 2.05 | 27.60 |
| 223 | 2.05 | 17.62 |
| 224 | 2.08 | 10.13 |
| 225 | 2.08 | 9.10 |
| 226 | 2.09 | 6.14 |
| 227 | 2.11 | 3.47 |
| 228 | 2.11 | 21.43 |
| 229 | 2.13 | 12.84 |
| 230 | 2.13 | 15.30 |
| 231 | 2.13 | 33.82 |
| 232 | 2.17 | 20.77 |
| 233 | 2.18 | 7.80 |
| 234 | 2.19 | 3.38 |
| 235 | 2.22 | 44.87 |
| 236 | 2.25 | 50.62 |
| 237 | 2.25 | 32.27 |
| 238 | 2.29 | 10.63 |
| 239 | 2.32 | 1.89 |
| 240 | 2.33 | 2.73 |
| 241 | 2.37 | 11.77 |
| 242 | 2.38 | 19.79 |
| 243 | 2.38 | 26.27 |
| 244 | 2.39 | 5.67 |
| 245 | 2.39 | 6.35 |
| 246 | 2.43 | 6.14 |
| 247 | 2.45 | 16.53 |
| 248 | 2.51 | 2.40 |
| 249 | 2.55 | 15.80 |
| 250 | 2.58 | 21.30 |
| 251 | 2.59 | 10.95 |
| 252 | 2.60 | 26.21 |
| 253 | 2.62 | 17.89 |
| 254 | 2.64 | 15.10 |
| 255 | 2.65 | 31.65 |
| 256 | 2.69 | 10.52 |
| 257 | 2.70 | 31.24 |
| 258 | 2.74 | 7.62 |
| 259 | 2.75 | 13.87 |
| 260 | 2.77 | 14.54 |
| 261 | 2.83 | 9.46 |
| 262 | 2.83 | 11.87 |
| 263 | 2.92 | 39.09 |
| 264 | 2.93 | 19.66 |
| 265 | 2.94 | 23.23 |
| 266 | 2.98 | 14.94 |
| 267 | 3.04 | 36.49 |
| 268 | 3.05 | 13.78 |
| 269 | 3.13 | 44.12 |
| 270 | 3.14 | 47.20 |
| 271 | 3.15 | 4.80 |
| 272 | 3.18 | 10.90 |
| 273 | 3.21 | 14.41 |
| 274 | 3.32 | 6.15 |
| 275 | 3.32 | 12.58 |
| 276 | 3.32 | 18.30 |
| 277 | 3.36 | 39.88 |
| 278 | 3.40 | 17.41 |
| 279 | 3.43 | 29.04 |
| 280 | 3.51 | 14.48 |
| 281 | 3.56 | 46.88 |
| 282 | 3.57 | 19.12 |
| 283 | 3.61 | 47.93 |
| 284 | 3.72 | 19.21 |
| 285 | 3.78 | 9.55 |
| 286 | 3.79 | 14.18 |
| 287 | 3.81 | 20.55 |
| 288 | 3.88 | 3.45 |
| 289 | 3.95 | 50.31 |
| 290 | 3.95 | 16.89 |
| 291 | 3.97 | 38.19 |
| 292 | 4.12 | 20.01 |
| 293 | 4.16 | 18.43 |
| 294 | 4.19 | 46.46 |
| 295 | 4.57 | 28.03 |
| 296 | 4.62 | 14.30 |
| 297 | 4.65 | 19.81 |
| 298 | 4.66 | 20.45 |
| 299 | 4.67 | 11.64 |
| 300 | 4.81 | 26.35 |
| 301 | 4.81 | 26.79 |
| 302 | 4.86 | 17.24 |
| 303 | 4.87 | 19.44 |
| 304 | 4.93 | 23.54 |
| 305 | 4.95 | 22.95 |
| 306 | 4.97 | 13.94 |
| 307 | 5.02 | 5.55 |
| 308 | 5.08 | 8.69 |
| 309 | 5.19 | 14.87 |
| 310 | 5.29 | 32.75 |
| 311 | 5.40 | 26.77 |
| 312 | 5.55 | 39.08 |
| 313 | 5.55 | 25.57 |
| 314 | 5.75 | 38.33 |
| 315 | 5.91 | 7.32 |
| 316 | 6.04 | 28.62 |
| 317 | 6.19 | 32.43 |
| 318 | 6.26 | 56.84 |
| 319 | 6.50 | 40.75 |
| 320 | 6.60 | 34.24 |
| 321 | 6.86 | 55.67 |
| 322 | 7.23 | 39.27 |
| 323 | 7.41 | 13.33 |
| 324 | 7.47 | 50.87 |
| 325 | 7.87 | 51.06 |
| 326 | 8.05 | 31.95 |
| 327 | 8.21 | 21.07 |
| 328 | 8.56 | 36.33 |
| 329 | 8.65 | 33.56 |
| 330 | 8.66 | 46.26 |
| 331 | 8.67 | 11.86 |
| 332 | 8.89 | 50.59 |
| 333 | 9.58 | 38.57 |
| 334 | 9.87 | 9.33 |
| 335 | 10.05 | 7.94 |
| 336 | 10.07 | 24.30 |
| 337 | 10.15 | 26.38 |
| 338 | 10.44 | 18.36 |
| 339 | 10.65 | 45.48 |
| 340 | 10.65 | 45.63 |
| 341 | 11.08 | 55.56 |
| 342 | 11.54 | 11.46 |
| 343 | 11.85 | 21.90 |
| 344 | 13.04 | 34.03 |
| 345 | 13.49 | 20.31 |
| 346 | 13.52 | 45.34 |
| 347 | 13.92 | 34.75 |
| 348 | 14.47 | 35.85 |
| 349 | 15.55 | 26.02 |
| 350 | 20.27 | 44.44 |
| 351 | 22.18 | 37.34 |
| 352 | 22.88 | 46.27 |
| 353 | 23.63 | 24.33 |
| 354 | 23.64 | 38.76 |
| 355 | 25.86 | 4.96 |
| 356 | 29.04 | 18.15 |
| 357 | 1.09 | 39.93 |
| 358 | 0.25 | 28.93 |
| 359 | 0.104 | 3.50 |
| 360 | 0.07 | 2.33 |
| 361 | 0.08 | 10.84 |
| 362 | 0.05 | 4.60 |
| 363 | 0.03 | 4.49 |
| 364 | 0.07 | 3.97 |
| 365 | 0.33 | 38.65 |
| 366 | 0.15 | 10.16 |
| 367 | 0.07 | 4.77 |
| 368 | 0.09 | 2.93 |
| 369 | 0.16 | 4.36 |
| 370 | 0.13 | 5.80 |
| 371 | 0.27 | 4.57 |
| 372 | 0.09 | 5.64 |
| 373 | 0.013 | 0.24 |
| 374 | 0.08 | 4.17 |
| 375 | 1.32 | 53.78 |
| 376 | 0.06 | 2.02 |
| 377 | 1.08 | 9.37 |
| 378 | 0.38 | 18.64 |
| 379 | 0.12 | 2.96 |
| 380 | 0.84 | 41.54 |
| 381 | 3.37 | 16.99 |
| 382 | 0.35 | 22.41 |
| 383 | 2.58 | 20.27 |
| 384 | 0.24 | 23.30 |
| 385 | 1.69 | 20.76 |
| 386 | 0.14 | 14.12 |
| 387 | 0.08 | 2.76 |
| 388 | 0.13 | 10.17 |
| 389 | 0.09 | 3.75 |
| 390 | 0.13 | 1.80 |
| 391 | 0.05 | 5.50 |
| 392 | 0.13 | 6.90 |
| 393 | 0.13 | 3.84 |
| 394 | 0.19 | 4.62 |
| 395 | 0.12 | 3.20 |
| 396 | 0.11 | 6.78 |
| 397 | 0.98 | 42.55 |
| 398 | 0.08 | 3.29 |
| 399 | 0.15 | 3.88 |
| 400 | 0.11 | 15.39 |
| 401 | 0.04 | 3.37 |
| 402 | 0.07 | 12.41 |
| 403 | 0.15 | 7.47 |
| 404 | 0.10 | 14.35 |
| 405 | 0.11 | 3.42 |
| 406 | 0.13 | 6.01 |
| 407 | 0.26 | 8.55 |
| 408 | 0.05 | 3.44 |
| 409 | 0.20 | 26.77 |
| 410 | 0.13 | 4.19 |
| 411 | 0.04 | 19.03 |
| 412 | 0.05 | 48.29 |
| 413 | 0.10 | 25.63 |
| 414 | 0.03 | 26.18 |
| 415 | 0.18 | 11.94 |
| 416 | 0.07 | 2.58 |
| 417 | 0.18 | 27.01 |
| 418 | 0.18 | 5.97 |
| 419 | 0.32 | 12.70 |
| 420 | 0.12 | 6.55 |
| 421 | 0.13 | 2.33 |
| 422 | 0.07 | 11.49 |
| 423 | 0.18 | 15.42 |
| 424 | 0.04 | 1.13 |
| 425 | 0.57 | 51.95 |
| 426 | 1.62 | 32.44 |
| 427 | 0.16 | 2.64 |
| 428 | 0.07 | 2.94 |
| 429 | 0.19 | 41.64 |
| 430 | 0.11 | 19.84 |
| 431 | 0.17 | 27.49 |
| 432 | 0.07 | 4.73 |
| 433 | 0.21 | 7.43 |
| 434 | 0.06 | 2.11 |
| 435 | 0.08 | 7.52 |
| 436 | 0.08 | 6.04 |
| 437 | 0.27 | 9.67 |
| 438 | 0.04 | 2.70 |
| 439 | 1.47 | 25.62 |
| 440 | 0.21 | 3.53 |
| 441 | 0.17 | 10.73 |
| 442 | 0.12 | 3.78 |
| 443 | 0.22 | 6.45 |
| 444 | 0.13 | 6.31 |
| 445 | 0.56 | 33.52 |
| 446 | 0.73 | 19.28 |
| 447 | 0.08 | 2.31 |
| 448 | 0.13 | 4.50 |
| 449 | 0.10 | 2.93 |
| 450 | 0.13 | 4.97 |
| 451 | 0.16 | 7.79 |
| 452 | 0.18 | 13.03 |
| 453 | 0.35 | 43.95 |
| 454 | 0.14 | 16.15 |
| 455 | 0.84 | 32.43 |
| 456 | 0.23 | 17.80 |
| 457 | 0.250 | 21.55 |
| 458 | 0.13 | 15.17 |
| 459 | 0.18 | 7.06 |
| 460 | 0.24 | 12.18 |
| 461 | 0.13 | 9.34 |
| 462 | 0.18 | 13.26 |
| 463 | 0.42 | 43.64 |
| 464 | 0.14 | 3.07 |
| 465 | 0.14 | 7.25 |
| 466 | 1.19 | 14.54 |
| 467 | 1.07 | 14.51 |
| 468 | 0.13 | 14.51 |
| 469 | 0.24 | 26.09 |
| 470 | 0.98 | 37.45 |
| 471 | 0.50 | 15.00 |
| 472 | 0.11 | 5.63 |

### Biological Example 2

### MDR1-MDCK Bidirectional Permeability Assay

The blood-brain barrier (BBB) is composed of brain capillary endothelial cells, which are characterized by highly developed tight junctions. The BBB plays a key role in the brain penetration of drugs and is an obstacle to the discovery of drugs where the drug target is in the central nervous system (CNS). P-Glycoproteins (P-gp, MDR1) are highly expressed at the BBB and serve to actively efflux drugs out of the brain and therefore may limit brain penetration of drugs to the desired target. A bidirectional permeability assay using Madin Darby canine kidney cells expressing multi-drug resistance gene 1 (MDCK-MDR1) is routinely used to evaluate discovery compounds' BBB permeability and drug efflux toward the advancement of discovery compounds with brain penetration.

MDCK-MDR1 Cells (NIH cell line) were obtained from the National Institutes of Health. MDCK-MDR1 cells were diluted to 1.56 million cells/mL (NIH) with culture medium and 50 μL of cell suspension were dispensed into the filter wells of a 96-well HTS Transwell plate. Cells were cultivated for 4-8 days in a cell culture incubator at 37° C., 5% CO₂, 95% relative humidity. Cell culture medium was replaced every other day, beginning no later than 24 hours after the initial plating. The permeability assay used buffer was Hanks' balanced salt solution containing 10 mM HEPES at a pH of 7.4. The dosing solution concentration was 1 μM for the test article in the assay buffer. Cell monolayers were dosed on the apical side (A-to-B) or basolateral side (B-to-A) and incubated at 37° C. under an atmosphere of 5% CO₂ in a humidified incubator. Samples were taken from the donor and receiver chambers at 120 minutes. Each determination was performed in duplicate. After the 120 min transport period, the flux of lucifer yellow was also measured for each monolayer to ensure no damage was inflicted to the cell monolayers during the transport period. All samples were assayed by LC-MS/MS (Waters XSelect HSS T3 C18, 2.5 m, 2.1×50 mm) using electrospray ionization with 0.1% formic acid in water (mobile phase A) and 0.1% formic acid in acetonitrile (mobile phase B) as mobile phases. The apparent permeability coefficient (Papp), in units of cm per second, was calculated for MDCK-MDR1 Cells (NIH cell line) drug transport assays using the following equation:

$$Papp = ( {V}_{A} \times {[ drug ]}_{acceptor} ) / ( Area \times Time \times {[ drug ]}_{initial , donor} )$$

Where V_{A} is the volume (in mL) in the acceptor well, Area is the surface area of the membrane (0.143 cm² for Transwell-96 Well Permeable Supports), and time is the total transport time in seconds.

$$Efflux ratio ( ER ) is defined as {P}_{app} ( B - to - A ) / {P}_{app} ( A - to - B ) .$$

The leakage of Lucifer Yellow (LY), in unit of percentage (%), can be calculated using the following equation:

$$\% LY leakage = 100 \times {[ LY ]}_{acceptor} / ( {[ LY ]}_{donor} + {[ LY ]}_{acceptor} )$$

LY leakage of <1% is acceptable to indicate the well-qualified MDCK-MDR1(NIH) monolayer.

The following comparator compounds were made using the procedures described above.

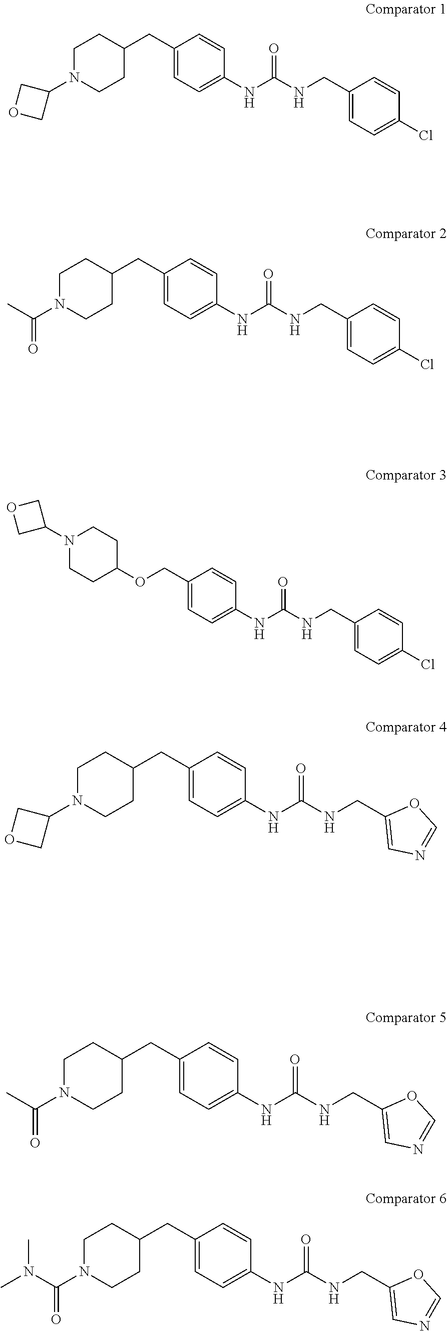 [SMILES: O=C(NCc1ccc(Cl)cc1)Nc1ccc(CC2CCN(C3COC3)CC2)cc1 ; CC(=O)N1CCC(Cc2ccc(NC(=O)NCc3ccc(Cl)cc3)cc2)CC1 ; O=C(NCc1ccc(Cl)cc1)Nc1ccc(COC2CCN(C3COC3)CC2)cc1 ; O=C(NCc1cnco1)Nc1ccc(CC2CCN(C3COC3)CC2)cc1 ; CC(=O)N1CCC(Cc2ccc(NC(=O)NCc3cnco3)cc2)CC1 ; CN(C)C(=O)N1CCC(Cc2ccc(NC(=O)NCc3cnco3)cc2)CC1]

Results are shown in Table B.

TABLE B

| Compound No. | PappAB | Efflux ratio |
|---|---|---|
| 43 | 5.4 | 0.92 |
| Comparator 1 | 1.85 | 14.7 |
| 40 | 3.4 | 1.9 |
| Comparator 2 | 0.77 | 21.4 |
| 232 | 9.8 | 1.5 |
| Comparator 3 | 2.1 | 24.5 |
| 80 | 13.3 | 2.8 |
| Comparator 4 | 1.0 | 19.7 |
| 31 | 6.6 | 7.2 |
| Comparator 5 | 0.7 | 27.2 |
| 234 | 9.80 | 1.52 |
| 8 | 9.77 | 5.41 |
| 452 | 9.47 | 4.01 |
| 7 | 9.16 | 3.09 |
| 464 | 9.11 | 2.19 |
| 70 | 9.10 | 1.04 |
| 35 | 8.31 | 2.90 |
| 376 | 8.06 | 4.11 |
| 26 | 7.93 | 3.82 |
| 27 | 7.68 | 1.99 |
| 31 | 6.63 | 7.25 |
| 30 | 6.31 | 4.07 |
| Comparator 6 | 0.5 | 64 |
| 54 | 5.76 | 5.79 |
| 23 | 6.30 | 2.00 |
| 452 | 5.82 | 4.44 |
| 32 | 5.57 | 3.32 |
| 43 | 5.39 | 0.92 |
| 5 | 5.32 | 7.02 |
| 414 | 4.58 | 3.96 |
| 18 | 4.52 | 7.34 |
| 65 | 3.89 | 6.09 |
| 469 | 3.72 | 4.04 |
| 449 | 3.53 | 5.74 |
| 40 | 3.38 | 1.93 |
| 424 | 3.37 | 7.46 |
| 395 | 2.81 | 5.02 |
| 21 | 2.79 | 3.74 |
| 51 | 13.82 | 1.23 |
| 132 | 13.54 | 2.26 |
| 59 | 13.26 | 1.11 |
| 4 | 11.11 | 3.27 |

### Biological Example 3

### Brain Exposure Assay in Mice

This protocol was used to determine the brain-to-plasma ratio in vivo in mice post oral administration of test compounds. Mice (C57BL/6JNIFDC, male, non-fasted, 18-30 g body weight, 3 mice for each timepoint) were dosed orally at 100 mg/kg (10 mg test compound/mL formulation, 10 mL dosing volume/kg) with plasma and brain tissues obtained at 2- and 6-hours post-administration. The oral dosing formulation composition used was 10% N,N-dimethylacetamide:20% propylene glycol:70% of 40% aqueous 2-hydroxypropyl-β-cyclodextrin.

For the isolation of mouse plasma from dosed animals, approximately 0.3 mL blood was collected by orbital sinus bleed and centrifuged (4000 g, 5 minutes, 4° C.). The plasma samples were stored frozen at −75±15° C. until further processing.

For the isolation of brain tissue from dosed animals, whole brain tissues were collected from euthanized and fully exsanguinated mice. The tissues were quickly rinsed with distilled water, dried with absorbent paper, followed by rapid freezing on dry ice and then storage at −75±15° C. until further processing. Brain tissue samples obtained from frozen whole brain were weighed and the samples homogenized using three equivalents of distilled water (3 mL per gram of brain tissue).

Standard and quality control samples were prepared by adding 3 μL of the test compound dimethyl sulfoxide (DMSO) stock solutions to blank plasma (30 μL) and blank brain homogenate aliquots (30 μL). Standard curves for brain and plasma were generated over a 1 to 1,000 ng/g test compound concentration range. For the extraction of test compound from plasma and brain tissue homogenate samples, a solution of acetonitrile containing internal standard (400 L) was added to 30 μL of samples with added 3 μL of DMSO. The samples then were vortex-mixed followed by centrifugation at 4000 rpm (4° C., 15 minutes). The resultant supernatants were diluted 5-fold with distilled water. Finally, 10 μL of the diluted supernatant was injected onto an LC-MS/MS instrument for quantitative analysis of test compound.

The brain tissue concentration was calculated by multiplying the concentration of drug detected in the homogenized brain tissue sample by a dilution factor of 4.

The brain-to-plasma ratio was calculated by the relationship: Brain-to-plasma ratio=C_{brain}/Cₚₗₐₛₘₐ; where C_{b} is the concentration in brain tissue and Cₚ is the concentration in plasma at the corresponding timepoint.

Kp is the total brain-plasma concentration ratio; Kₚ=C_{brain}/Cₚₗₐₛₘₐ.

Kp, uu is the unbound brain-to-plasma partition coefficient (K_{p,uu,brain}). It is a measure of the extent of the distribution equilibrium of a compound between the unbound (free) fractions in brain and in blood plasma; K_{p, uu brain}=C_{u, brain}/C_{u, plasma}.

A compound is estimated as “brain penetrant” if its brain-to-plasma concentration ratio is >0.04, as cerebral blood volume is estimated at 4% of total brain volume (Shaffer CL (2010), Defining Neuropharmacokinetic Parameters in CNS Drug Discovery to Determine Cross-Species Pharmacologic Exposure-Response Relationships, Annual Reports in Medicinal Chemistry, 45:55-70, https://doi.org/10.1016/S0065-7743(10)45004-6).

| | | Brain | | | | | | |
|---|---|---|---|---|---|---|---|---|
| | | tissue | C_{plasma, total} | C_{brain, total} | C_{plasma, free} | C_{brain, free} | | |
| | PPB | binding | (μM), | (μM), | (μM), | (μM), | Kₚ | K_{p, uu} |
| Co. | (mouse) | (mouse) | 100 mg/kg, | 100 mg/kg, | 100 mg/kg, | 100 mg/kg, | 2 hr, | 2 hr, |
| No. | % unbound | % unbound | 2 hr, 6 hr | 2 hr, 6 hr | 2 hr, 6 hr | 2 hr, 6 hr | 6 hr | 6 hr |
| 54 | 4.5 | 2.2 | 43.0, | 27.3, | 1.94, | 0.60, | 0.63, | 0.32, |
| | | | 8.3 | 4.6 | 0.77 | 0.01 | 0.55 | 0.27 |
| 80 | 20 | 27 | 25.7, | 14.1, | 5.14, | 3.8, | 0.55, | 0.74, |
| | | | 5.3 | 3.0 | 1.06 | 0.81 | 0.57 | 0.77 |
| 30 | 4.9 | 8.5 | 45.0, | 18.3, | 2.20, | 1.56, | 0.41, | 0.71, |
| | | | 11.8 | 4.8 | 0.58 | 0.41 | 0.41 | 0.71 |
| 18 | 7.6 | 14.8 | 75.3, | 9.4, | 5.72, | 1.41, | 0.12, | 0.25, |
| | | | 25.4 | 3.4 | 1.93 | 0.51 | 0.13 | 0.25 |
| 27 | 3.7 | 5.3 | 42.1, | 12.0, | 1.55, | 0.64, | 0.29, | 0.41, |
| | | | 5.9 | 1.5 | 0.22 | 0.07 | 0.25 | 0.32 |
| 7 | 5.7 | 20.4 | 89.5, | 20.8, | 5.10, | 4.24, | 0.23, | 0.83, |
| | | | 20.2 | 4.0 | 1.15 | 0.82 | 0.20 | 0.71 |
| 449 | 2.2 | 3.3 | 59.4, | 25.0, | 1.30, | 0.83, | 0.42, | 0.64, |
| | | | 18 | 8.4 | 0.39 | 0.28 | 0.47 | 0.72 |
| 376 | 2.4 | 13.2 | 104, | 16.5, | 2.48, | 2.2, | 0.16, | 0.89, |
| | | | 45.5 | 7.5 | 1.09 | 1.0 | 0.16 | 0.92 |
| 400 | 4.3 | 5.4 | 13.5, | 5.8, | 0.58, | 0.31, | 0.43, | 0.53, |
| | | | 3.1 | 1.4 | 0.14 | 0.08 | 0.43 | 0.57 |
| 399 | 1.0 | 1.2 | 44, | 19.5, | 0.44, | 0.23, | 0.44, | 0.52, |
| | | | 26.8 | 11.5 | 0.27 | 0.14 | 0.43 | 0.52 |
| 424 | 16.5 | 9.2 | 11.7, | 2.6, | 1.94, | 0.24, | 0.22, | 0.12, |
| | | | 1.9 | 0.5 | 0.31 | 0.05 | 0.26 | 0.16 |
| 51 | 6.5 | 7.8 | 48.8, | 18.5, | 3.17, | 1.44, | 0.38, | 0.45, |
| | | | 11.0 | 4.5 | 0.72 | 0.35 | 0.41 | 0.49 |
| 80 | 19.5 | 26.9 | 25.7, | 14.1, | 5.01, | 3.8, | 0.55, | 0.74, |
| | | | 5.3 | 3.0 | 1.03 | 0.81 | 0.57 | 0.77 |
| Compar- | 52.3 | 51.8 | 40.1, | 3.2, | 20.6, | 1.66, | 0.08, | 0.08, |
| ator 4 | | | 7.3 | 0.7 | 3.82 | 0.40 | 0.10 | 0.10 |

All publications, patent applications, patents, and other references mentioned herein are expressly incorporated by reference in their entireties, to the same extent as if each were incorporated by reference individually.

It is to be understood that, while the disclosure provided herein has been described in conjunction with the above embodiments, the foregoing description and examples are intended to illustrate and not limit the scope of the disclosure. Other aspects, advantages, and modifications within the scope of the disclosure will be apparent to those skilled in the art to which the disclosure pertains.

1. A compound of Formula (I) 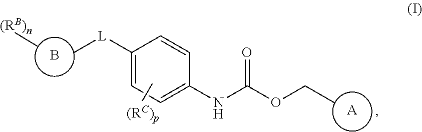
or a pharmaceutically acceptable salt thereof, wherein:
Ring A is: i) 5- to 6-membered heteroaryl optionally substituted with 1 to 4 R^{A}, or 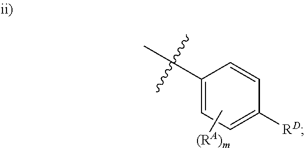
each R^{A} is independently selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
R^{D} is selected from the group consisting of:
halogen;
cyano;
C₁-C₆ alkyl optionally substituted with 1 to 3 independently selected halogens or —OH;
—O(C₁-C₆ alkyl) optionally substituted with 1 to 3 independently selected halogens; and
—C(O)NR^{A1}R^{A2}, wherein R^{A1} and R^{A2} are each independently hydrogen or C₁-C₆ alkyl;
L is selected from the group consisting of a bond, C₁-C₆ alkylene,
#—O—(C₁-C₆ alkylene)-$, #—C(O)—(C₁-C₆ alkylene)-$,
#—(C₁-C₆ alkylene)-C(O)—$, #—N(R^{L})—(C₁-C₆ alkylene)-$, #—(C₁-C₆ alkylene)-N(R^{L})—$,
#—(C₁-C₆ alkylene)-N(R^{L})—(C₁-C₆ alkylene)-$,
#—C(O)—N(R^{L})—(C₁-C₆ alkylene)-$, #—(C₁-C₆ alkylene)-C(O)—N(R^{L})—$,
#—N(R^{L})—C(O)—CH₂-$, #—(C₁-C₆ alkylene)-N(R^{L})—C(O)—$,
#—(C₁-C₆ alkylene)-C(O)—N(R^{L})—(C₁-C₆ alkylene)-$,
#—(C₁-C₆ alkylene)-N(R^{L})—C(O)—(C₁-C₆ alkylene)-$, #—(C₁-C₆ alkylene)-N(R^{L})—S(O)₂-$,
#—N(R^{L})—S(O)₂—(C₁-C₆ alkylene)-$, #—(C₁-C₆ alkylene)-S(O)₂—N(R^{L})—$, #—S(O)₂—N(R^{L})—(C₁-C₆ alkylene)-$, #—S(O)₂—N(R^{L})—$, and #—N(C₁-C₆ alkyl)-S(O)₂-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl;
Ring B is 4- to 10-membered heterocycloalkyl, 3- to 8-membered cycloalkyl, 5- to 6-membered heteroaryl, or phenyl;
each R^{B} is independently selected from the group consisting of:
halogen;
—OH;
oxo;
cyano;
—O(C₁-C₆ alkyl) optionally substituted with phenyl or 1 to 3 independently selected halogens;
—C(O)(C₁-C₆ alkyl);
—C(O)O(C₁-C₆ alkyl);
phenyl;
5- to 6-membered heteroaryl;
4- to 8-membered heterocycloalkyl;
3- to 8-membered cycloalkyl;
—C(O)(3- to 8-membered cycloalkyl) optionally substituted with 1 to 3 independently selected halogens;
—C(O)(4- to 8-membered heterocycloalkyl) optionally substituted with 1 to 3 independently selected halogens;
—S(O)₂(C₁-C₆ alkyl);
—S(O)₂(3- to 8-membered cycloalkyl);
—S(O)₂(4- to 8-membered heterocycloalkyl);
—C(O)NR^{B1}R^{B2};
—S(O)₂NR^{B1}R^{B2};
—NR^{C1}S(O)₂NR^{B1}R^{B2};
—(C═N—R^{C1})—NR^{B1}R^{B2};
—NR^{C1}—(C═N—R^{C1})—NR^{B1}R^{B2}; —NR^{C1}—(C═N—CN)—NR^{B1}R^{B2}; and C₁-C₆ alkyl optionally substituted with 1 to 5 substituents independently selected from the group consisting of halogen, —OH, 4- to 8-membered heterocycloalkyl, and —O(C₁-C₆ alkyl);
wherein R^{C1}, R^{B1} and R^{B2} are each independently hydrogen or C₁-C₆ alkyl;
m is 0, 1, 2, 3, or 4;
n is 0, 1, 2, 3, 4, or 5;
R^{C} is halogen, cyano, C₁-C₆ alkyl, OH, —O(C₁-C₆ alkyl), or 3- to 8-membered cycloalkyl; and
p is 0, 1, 2, 3, or 4;
wherein:
a) when L is a bond, ring B is selected from the group consisting of 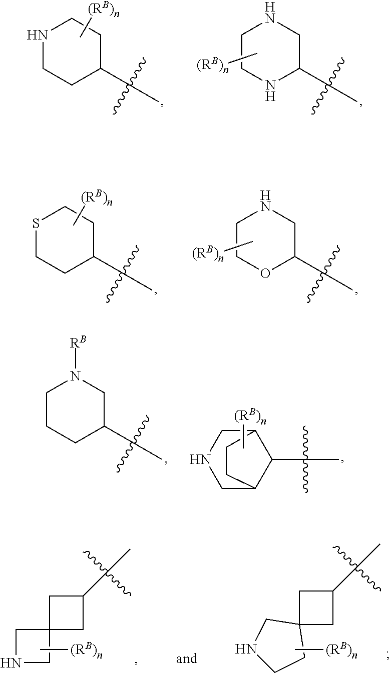
b) when ring B is pyridin-4-yl and A is phenyl, R^{D} is selected from the group consisting of carbamoyl, chloro, hydroxymethyl, difluoromethyl, methoxy, and cyano; and
c) when L is —CH₂—CH₂—, n is 0, 1, or 2.

2. The compound of claim 1, or a pharmaceutically acceptable salt thereof, wherein Ring A is

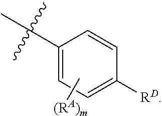

3. The compound of claim 1, or a pharmaceutically acceptable salt thereof, wherein R^{D} is halogen or —O(C₁-C₆ alkyl) optionally substituted with 1 to 3 independently selected halogens.

4. The compound of claim 1, or a pharmaceutically acceptable salt thereof, wherein R^{D} is halogen.

5. The compound of claim 1, or a pharmaceutically acceptable salt thereof, wherein R^{D} is fluorine.

6. The compound of claim 1, or a pharmaceutically acceptable salt thereof, wherein R^{D} is chlorine.

7. The compound of claim 1, or a pharmaceutically acceptable salt thereof, wherein Ring A is 5- to 6-membered heteroaryl optionally substituted with 1 to 4 R^{A}.

8. The compound of claim 1, or a pharmaceutically acceptable salt thereof, wherein Ring A is 5-membered heteroaryl optionally substituted with 1 to 4 R^{A}.

9. The compound of claim 1, or a pharmaceutically acceptable salt thereof, wherein Ring A is oxazolyl, pyrazolyl, thiazolyl, isothiazolyl, or isoxazolyl, each of which is optionally substituted with 1 to 3 R^{A}.

10. The compound of claim 1, or a pharmaceutically acceptable salt thereof, wherein Ring A is oxazolyl optionally substituted with 1 to 2 R^{A}.

11. The compound of claim 1, or a pharmaceutically acceptable salt thereof, wherein Ring A is oxazol-5-yl optionally substituted with 1 to 2 R^{A}.

12. The compound of claim 1, or a pharmaceutically acceptable salt thereof, wherein Ring A is thiazolyl optionally substituted with 1 to 2 R^{A}.

13. The compound of claim 1, or a pharmaceutically acceptable salt thereof, wherein Ring A is 6-membered heteroaryl optionally substituted with 1 to 4 R^{A}.

14. The compound of claim 1, or a pharmaceutically acceptable salt thereof, wherein Ring A is pyridinyl, pyridazinyl, or pyrimidinyl, each of which is optionally substituted with 1 to 4 R^{A}.

15. The compound of claim 1, or a pharmaceutically acceptable salt thereof, wherein Ring A is pyridinyl optionally substituted with 1 to 4 R^{A}.

16. The compound of claim 1, or a pharmaceutically acceptable salt thereof, wherein Ring A is pyridin-4-yl optionally substituted with 1 to 4 R^{A}.

17. The compound of claim 1, or a pharmaceutically acceptable salt thereof, wherein each R^{A} is independently selected from the group consisting of halogen and C₁-C₆ alkyl.

18. The compound of claim 1, or a pharmaceutically acceptable salt thereof, wherein Ring B is 4- to 8-membered heterocycloalkyl, 3- to 8-membered cycloalkyl, or 5- to 6-membered heteroaryl.

19. The compound of claim 1, or a pharmaceutically acceptable salt thereof, wherein Ring B is pyridinyl, piperazinyl, morpholinyl, piperidinyl, pyrrolidinyl, oxazolyl, or pyrazolyl.

20. The compound of claim 1, or a pharmaceutically acceptable salt thereof, wherein Ring B is pyridinyl, piperazinyl, morpholinyl, piperidinyl, or pyrrolidinyl.

21. The compound of claim 1, or a pharmaceutically acceptable salt thereof, wherein Ring B is

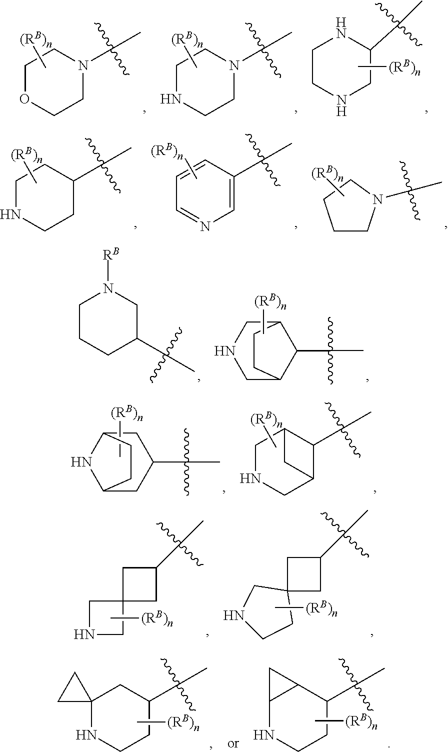

22. The compound of claim 1, or a pharmaceutically acceptable salt thereof, wherein each R^{B} is independently selected from the group consisting of:
halogen;
oxo;
—O(C₁-C₆ alkyl) optionally substituted with phenyl or 1 to 3 independently selected halogens;
C₁-C₆ alkyl optionally substituted with 1 to 5 substituents independently selected from halogen, —OH, 4- to 8-membered heterocycloalkyl, and —O(C₁-C₆ alkyl);
—S(O)₂(C₁-C₆ alkyl);
—S(O)₂(4- to 8-membered heterocycloalkyl);
—S(O)₂(3- to 8-membered cycloalkyl);
—S(O)₂NR^{B1}R^{B2};
—C(O)NR^{B1}R^{B2};
—C(O)(C₁-C₆ alkyl);
—C(O)O(C₁-C₆ alkyl);
—C(O)(3- to 8-membered cycloalkyl) optionally substituted with 1 to 3 independently selected halogens;
—C(O)(4- to 8-membered heterocycloalkyl) optionally substituted with 1 to 3 independently selected halogens;
4- to 8-membered heterocycloalkyl; and
5- to 6-membered heteroaryl.

23. The compound of claim 1, or a pharmaceutically acceptable salt thereof, wherein each R^{B} is independently selected from the group consisting of oxo, —C(O)(C₁-C₆ alkyl), —C(O)NR^{B1}R^{B2}, —S(O)₂(C₁-C₆ alkyl), —S(O)₂NR^{B1}R^{B2}, unsubstituted C₁-C₆ alkyl, 4- to 8-membered heterocycloalkyl, and 5- to 6-membered heteroaryl.

24. The compound of claim 1, or a pharmaceutically acceptable salt thereof, wherein each R^{B} is independently selected from the group consisting of —O(C₁-C₄ alkyl) optionally substituted with phenyl or 1 to 3 independently selected halogens; and
C₁-C₄ alkyl optionally substituted with 1 to 5 substituents independently selected from halogen, —OH, 4- to 8-membered heterocycloalkyl, and —O(C₁-C₄ alkyl);

25. The compound of claim 1, or a pharmaceutically acceptable salt thereof, wherein each R^{B} is independently selected from the group consisting of oxo, —C(O)(C₁-C₃ alkyl), —C(O)N(Me)₂, —C(O)(4- to 8-membered heterocycloalkyl) optionally substituted with 1 to 3 independently selected halogens, —C(O)(3- to 8-membered cycloalkyl) optionally substituted with 1 to 3 independently selected halogens; —S(O)₂(C₁-C₃ alkyl), —S(O)₂N(Me)₂, —C(O)O(C₁-C₄ alkyl), —S(O)₂(4- to 8-membered heterocycloalkyl), —S(O)₂(3- to 4-membered cycloalkyl), methyl, oxetanyl, and pyridinyl.

26. The compound of claim 1, or a pharmaceutically acceptable salt thereof, wherein L is a bond, C₁-C₆ alk7ylene, #—C(O)—N(R^{L})—(C₁-C₆ alkylene)-$, #—C(O)—(C₁-C₆ alkylene)-$, or #—(C₁-C₆ alkylene)-C(O)—N(R^{L})—(C₁-C₆ alkylene)-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl.

27. The compound of claim 1, or a pharmaceutically acceptable salt thereof, wherein each C₁-C₆ alkylene of L is substituted with 1 to 3 substituents independently selected from the group consisting of halogen, —OH, and C₁-C₆ alkyl.

28. The compound of claim 1, or a pharmaceutically acceptable salt thereof, wherein each C₁-C₆ alkylene of L is substituted with 1 to 3 substituents independently selected from the group consisting of halogen and C₁-C₆ alkyl.

29. The compound of claim 1, or a pharmaceutically acceptable salt thereof, wherein each C₁-C₆ alkylene of L is substituted with 1 to 3 substituents independently selected from the group consisting of fluoro and methyl.

30. The compound of claim 1, or a pharmaceutically acceptable salt thereof, wherein each C₁-C₆ alkylene of L is unsubstituted.

31. The compound of claim 1, or a pharmaceutically acceptable salt thereof, wherein L is a bond, C₁-C₃ alkylene, or #—C(O)—N(R^{L})—(C₁-C₃ alkylene)-$, wherein #represents the attachment point to ring B, and $ represents the attachment point to the remainder of the molecule;
wherein each C₁-C₆ alkylene of L is optionally substituted with 1 to 3 substituents independently selected from the group consisting of halogen, —OH, and C₁-C₆ alkyl; and
wherein each R^{L} is independently hydrogen or C₁-C₆ alkyl.

32. The compound of claim 1, or a pharmaceutically acceptable salt thereof, wherein L is a bond, —CH₂—, —CH₂CH₂—, or

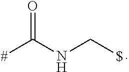

33. The compound of claim 1, or a pharmaceutically acceptable salt thereof, wherein m is 1.

34. The compound of claim 1, or a pharmaceutically acceptable salt thereof, wherein m is 0.

35. The compound of claim 1, or a pharmaceutically acceptable salt thereof, wherein n is 0, 1, or 2.

36. The compound of claim 1, or a pharmaceutically acceptable salt thereof, wherein R^{C} is fluoro.

37. The compound of claim 1, or a pharmaceutically acceptable salt thereof, wherein p is 0.

38. A compound selected from the group consisting of compounds 1-356 of Table 1, or a pharmaceutically acceptable salt thereof.

39. A compound selected from the group consisting of the compounds of Table 1, or a pharmaceutically acceptable salt thereof.

40. A compound selected from the group consisting of: 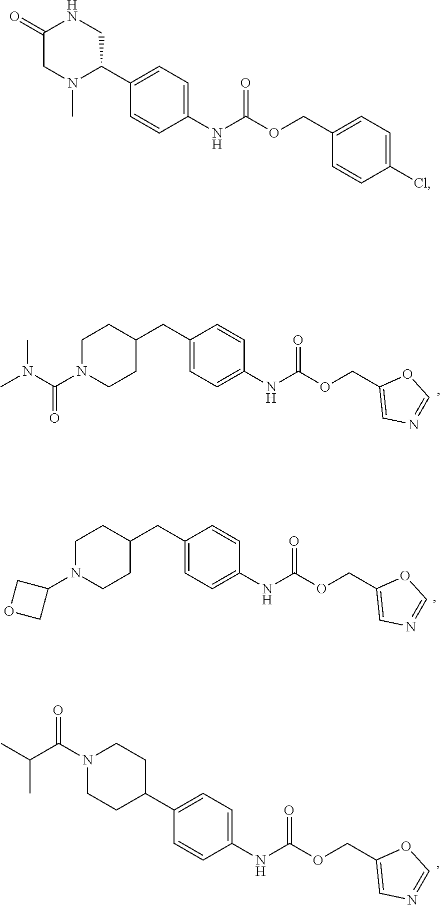 [SMILES: CN1CC(=O)NC[C@@H]1c1ccc(NC(=O)OCc2ccc(Cl)cc2)cc1 ; CN(C)C(=O)N1CCC(Cc2ccc(NC(=O)OCc3cnco3)cc2)CC1 ; O=C(Nc1ccc(CC2CCN(C3COC3)CC2)cc1)OCc1cnco1 ; CC(C)C(=O)N1CCC(c2ccc(NC(=O)OCc3cnco3)cc2)CC1] 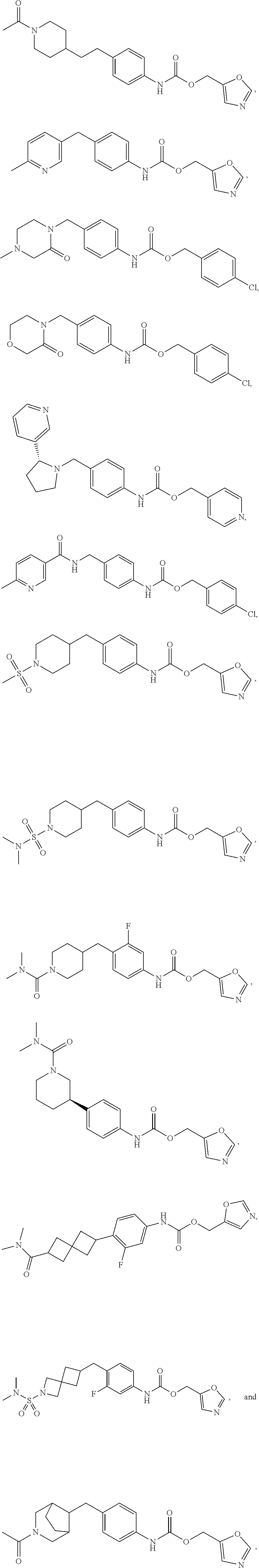
or a pharmaceutically acceptable salt thereof.

41. A pharmaceutical composition comprising a compound according to claim 1, or a pharmaceutically acceptable salt thereof, and a pharmaceutically acceptable-excipient.

42. A method of treating a disease or condition mediated by NAMPT activity in a subject in need thereof, comprising administering to the subject a compound of claim 1, or a pharmaceutically acceptable salt thereof.

43. The method of claim 42, wherein the disease or condition is selected from the group consisting of cancer, a hyperproliferative disease or condition, an inflammatory disease or condition, a metabolic disorder, a cardiac disease or condition, chemotherapy induced tissue damage, a renal disease, a metabolic disease, a neurological disease or injury, a neurodegenerative disorder or disease, diseases caused by impaired stem cell function, diseases caused by DNA damage, primary mitochondrial disorders, and a muscle disease or muscle wasting disorder.

44. The method of claim 42, wherein the disease or condition is selected from the group consisting of obesity, atherosclerosis, insulin resistance, type 2 diabetes, cardiovascular disease, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.
